SEQUENCE LISTING (2 sequences)

SEQ ID NO: 1 - protein, 163 aa, Homo sapiens
MADEEKLPPGWEKRMSRSSGRVYYFNHITNASQWERPSGNSSSGGKNGQGEPARVRCSHLLVKHSQSRRPSSWRQEKITRTKEEALELINGYIQKIKSGEEDFESLASQFSDCSSAKARGDLGAFSRGQMQKPFEDASFALRTGEMSGPVFTDSGIHIILRTE

SEQ ID NO: 2 - protein, 163 aa, Homo sapiens
MADEEKLPPGWEKAMSRSSGRVYYFNHITNASQWERPSGNSSSGGKNGQGEPARVRCSHLLVKHSQSRRPSSWRQEKITRTKEEALELINGYIQKIKSGEEDFESLASQFSDCSSAKARGDLGAFSRGQMQKPFEDASFALRTGEMSGPVFTDSGIHIILRTE

(19) United States
(12) Patent Application Publication
(10) Pub. No.: US 2004/0176912 A1
(43) Pub. Date: Sep. 9, 2004

(54) Methods for designing specific inhibitors for Pin1 proline isomerase and Pin1-related molecules

(75) Inventors: Janusz Sowadski, Boston, MA (US); Robert K. Suto, Maynard, MA (US)

Correspondence Address: LAHIVE & COCKFIELD, LLP., 28 STATE STREET, BOSTON, MA, 02109, US

(73) Assignee: Pintex Pharmaceuticals, Inc., Watertown, MA (US)

(21) Appl. No.: 10/379,115

(22) Filed: Mar. 3, 2003

Related U.S. Application Data
(60) Provisional application No. 60/361,227.

(51) Int. Cl. G06F 19/00; G01N 33/48; G01N 33/50

(52) U.S. Cl. 702/19

(57) ABSTRACT

This invention relates to methods for designing specific inhbitors of Pin1/parvulin family of peptidyl-prolyl cis-trans isomerases, particularly Pin1 and Pin1-related proteins, through the use of the structure coordinates and atomic details elucidated from the crystal structures of a mutant of Pin1, R14A, and co-complexes of R14A with various inhibitors. The invention further relates to inhibitors of Pin1 and Pin1-related proteins that are useful for therapeutic treatement of various disorders, and methods of using Pin1 inhibitors in pharmaceutical compositions. The invention further relates to the crystal structures of Pin1R14A alone, and of Pin1R14A in co-complex with molecules that bind to Pin1.

### RELATED APPLICATIONS

This application is claims priority to U.S. Provisional Application No. 60/361,227 filed Mar. 1, 2002. This application is related to U.S. Application No. 60/257,412, filed Dec. 22, 2000; U.S. application Ser. No. 09/726,464, filed Nov. 29, 2000; U.S. application Ser. No. 08/988,842, filed Dec. 11, 1997; U.S. application Ser. No. 10/027,864, filed Dec. 21, 2001; and WO 99/12962, published Mar. 8, 1999; PTZ-034, entitled “Pin1-Modulating Compounds and Methods of Use Thereof”, filed Mar. 3, 2003; PTZ-035, entitled “Pin1-Modulating Compounds and Methods of Use Thereof”, filed Mar. 3, 2003; PTZ-036, entitled “Pin1-Modulating Compounds and Methods of Use Thereof”, filed Mar. 3, 2003; U.S. Provisional Application Serial No. 60/361,246, filed Mar. 1, 2002; U.S. Provisional Application Serial No. 60/361,206, filed Mar. 1, 2002; and U.S. Provisional Application Serial No. 60/361,231, filed Mar. 1, 2002 the entire contents of which are expressly incorporated by reference.

### TECHNICAL FIELD OF THE INVENTION

This invention relates to methods for designing specific inhibitors of Pin1/parvulin family of peptidyl-prolyl cis-trans isomerases, particularly Pin1 and Pin1-related proteins, through the use of the structure coordinates and atomic details elucidated from the crystal structures of a mutant of Pin1, R14A, and co-complexes of R14A with various inhibitors. The invention further relates to the crystal structures of Pin1R14A alone, and of Pin1R14A in co-complexes with various inhibitors.

### BACKGROUND OF THE INVENTION

The peptidyl-prolyl cis-trans isomerases (PPIases), or rotamases, are a family of ubiquitous enzymes that catalyze the cis/trans isomerization of the peptide bond on the n-terminal side of proline residues in proteins (Hunter, Cell 92:141-142, 1998). PPIases are divided into three classes, cyclophilins (Cyps), FK-506 binding proteins (FKBPs) and the Pin1/parvulin class.

Cyclophilins and FKBPs are distinguished by their ability to bind the clinically immunosuppressive drugs cyclosporin and FK506, respectively (Schreiber, Science 251:283-7, 1991; Hunter, supra). Upon binding of these drugs, there are two common outcomes: inhibition of the PPIase activity and inhibition of the common target calcineurin. The inhibition of calcineurin phosphatase activity prevents lymphocytes from responding to antigen-induced mitogenic signals, thus resulting in immunosuppression. However, the inhibition of the PPIase activity is apparently unrelated to the immunosuppressive property of the drug/PPIase complexes. Even more surprisingly, deletion of all 8 known cyclophilins and 4 FKBPs in the same cells does not result in any significant phenotype (Dolinski et al., Proc. Natl. Acad. Sci. USA 94:13093-131098, 1997).

In contrast, members of the Pin1/parvulin class of PPIases bind neither of these immunosuppressive drugs, and are structurally unrelated to the other two classes of PPIases. Known members of the Pin1/parvulin class include Pins1-3 (Lu et al., Nature 380;544-547, 1996), Pin-L (Campbell et al., Genomics 44:157-162, 1997), parvulin (Rahfeld, et al., Proc. Natl. Acad. Sci. USA 93:447-451, 1996) and Ess1/Pft1 (Hanes et al., Yeast 5:55-72, 1989; and Hani, et al. FEBS Letts 365:198-202, 1995).

Pin1 is a highly conserved protein that catalyzes the isomerization of only phosphorylated Ser/Thr-Pro bonds (Rananathan, R. et al. (1997) Cell 89:875-86; Yaffe, et al. 1997, Science 278:1957-1960; Shen, et al. 1998, Genes Dev. 12:706-720; Lu, et al. 1999, Science 283:1325-1328; Crenshaw, et al. 1998, Embo J. 17:1315-1327; Lu, et al. 1999, Nature 399:784-788; Zhou, et al. 1999, Cell Mol. Life Sci. 56:788-806). In addition, Pin1 contains an N-terminal WW domain, which functions as a phosphorylated Ser/Thr-Pro binding module (Sudol, M. (1996) Prog. Biophys. Mol. Biol. 65:113-32). This phosphorylation-dependent interaction targets Pin1 to a subset of phosphorylated substrates, including Cdc25, Wee 1, Mytl, Tau-Rad4, and the C-terminal domain of RNA polymerase II large domain (Crenshaw, D. G., et al. (1998) Embo. J 17:1315-27; Shen, M. (1998) Genes Dev. 12:706-20; Wells, N. J. (1999) J. Cell. Sci. 112: 3861-71).

The specificity of Pin1 activity is essential for cell growth; depletion or mutations of Pin1 cause growth arrest, affect cell cycle checkpoints and induce premature mitotic entry, mitotic arrest and apoptosis in human tumor cells, yeast or Xenopus extracts (Lu, et al. 1996, Nature 380:544-547; Winkler, et al. 200, Science 287:1644-1647; Hani, et al. 1999. J. Biol. Chem. 274:108-116). In addition, Pin1 is dramatically overexpressed in human cancer samples and the levels of Pin1 are correlated with the aggressiveness of tumors. Moreover, inhibition of Pin1 by various approaches, including Pin1 antisense polynucleotides or genetic depletion, kills human and yeast dividing cells by inducing premature mitotic entry and apoptosis.

Thus, Pin 1-catalyzed prolyl isomerization regulates the conformation and function of these phosphoprotein substrates and facilitates dephosphorylation because of the conformational specificity of some phosphatases. Thus, Pin1-dependent peptide bond isomerization is a critical post-phosphorylation regulatory mechanism, allowing cells to turn phosphoprotein function on or off with high efficiency and specificity during temporally regulated events, including the cell cycle (Lu et al., supra).

Taken together, these results indicate that the Pin-1 subfamily of enzymes is a novel target for diseases characterized by uncontrolled cell proliferation, primarily malignancies. Therefore, there is an ongoing need for specific inhibitors of Pin1 and Pin1-related proteins, and for reliable methods of designing such inhibitors.

### SUMMARY OF THE INVENTION

The present invention provides a method for designing specific inhibitors of Pin1 proline isomerase. The method is based, at least in part, on the identification of specific interactions within the proline isomerase active site between non-specific inhibitors, substrate-based peptides, and solvent-based molecules to define an interaction template. This may be achieved through the crystallographic screening of low affinity nonspecific inhibitors found in publicly available libraries. This method has allowed for the identification of functional regions of the active site (FIG. 2).

Exemplary compounds used in the methods of the invention are presented in FIG. 3.

The conserved and nonconserved regions of the Pin1 proline isomerases are identified through structural and sequence alignment analysis and used to generate a specificity template. Once this information is provided, inhibitors of increased affinity and specificity are designed to interact with the defined templates. The conformation of the improved inhibition and specificity are confirmed through biochemical assays.

In one aspect, the invention pertains, at least in part, to a Pin1 polypeptide in the crystallized form having a substitution, insertion or deletion of one or more amino acids of the amino acid sequence set forth in SEQ ID NO:1, wherein the active site of the Pin1 polypeptide in the crystallized form is accessible to solvent and available for interaction with an inhibitor.

In a related embodiment, the substitution, insertion or deletion of one or more amino acids is within the WW-domain of the Pin1 polypeptide.

In more specific embodiments, the crystallized Pin1 polypeptide has an amino acid substitution at position 14 of the amino acid sequence set forth in SEQ ID NO:1. And in one particular embodiment, the substitution at position 14 is a nonpolar amino acid. In a more particular embodiment, this nonpolar amino acid is an alanine (SEQ ID NO:2).

In one embodiment of the invention, the Pin1 polypeptide having a substitution, insertion or deletion crystallizes in less than 7 days, less than 3 days, less than 2 days or preferably less than one day.

In another related embodiment, the Pin1 polypeptide having a substitution, insertion or deletion crystallizes such that the resolution of the determined X-ray crystallography structure is 3 Å or less or preferably 2 Å or less.

In one particular embodiment, the crystal comprising the Pin1 polypeptide having a substitution, insertion, or deletion has the space group P3₁21.

In one related embodiment, the Pin1 polypeptide having a substitution, insertion or deletion is a fragment of the Pin1 polypeptide.

In another related embodiment, the crystal comprising the Pin1 polypeptide having a substitution, insertion, or deletion can also contain a small molecule, or an inhibitor in the active site.

In another aspect the invention pertains to a method for designing or selecting an inhibitor of a Pin1 polypeptide. This method includes using a three dimensional structure of a crystallized Pin1 polypeptide having a substitution, deletion or insertion of one or more amino acids to form a Pin1 interaction template and using the Pin1 interaction template to design or select the Pin1 inhibitor.

In another related embodiment, the crystal comprising the Pin1 polypeptide having a substitution, insertion, or deletion can also contain a small molecule, or an inhibitor.

In a related embodiment, the three dimensional structure of more than one crystallized Pin1 polypeptide is used to form the interaction template.

In a related embodiment, the three dimensional structure of more than one crystallized Pin1 polypeptide with a small molecule within the active site of the polypeptide is used to form the Pin1 interaction template.

In another embodiment the method of designing or selecting an inhibitor uses the interaction template in conjunction with a specificity template.

In another embodiment, the method of designing or selecting an inhibitor involves contacting the inhibitor with a Pin1 polypeptide and a Pin1 substrate to determine the ability of the inhibitor to inhibit the Pin1 polypeptide.

In a related embodiment the method of designing or selecting an inhibitor involves determining the affinity and specificity of the inhibitor.

In another embodiment, the invention pertains to a method of designing inhibitors with the following steps:

(a) determining the three-dimensional structures of crystallized Pin1 polypeptides in complex with small molecules;

(b) employing the three-dimensional structures from step (a) to form an interaction template;

(c) employing sequence and structural alignments to identify conserved amino acids among proline isomerases and distinguishing those amino acids that are specific to Pin1 to define a specificity template;

(d) using the interaction template and the specificity template in conjunction with molecular modeling to design or select inhibitors specific to Pin1 or Pin 1-like proline isomerases; and

(e) determining the affinity and specificity of the inhibitors to Pin1.

In a one embodiment, the invention pertains to methods of using the three dimensional structures to design or select chemical entities or fragments that bind to at least one of the following portions of the Pin1 active site: the hydrophobic pocket, the cysteine/serine valley, the phosphate binding pocket, the substrate-entry groove, and the lip regions.

In a related embodiment the chemical entities or fragments that bind to the active site are covalently linked.

The interaction template of the present invention can be used to identify, select, and or design Pin1 modulators (e.g., inhibitors) having any one or more of the following moieties:

A-B-C-D-E

where A is a hydrophobic pocket interacting moiety; where B is a cysteine/serine valley interacting moiety; where C is a phosphate pocket interacting moiety; where D is a substrate-entry groove interacting moiety; and where E is a lip region interacting moiety.

In related embodiments, the modulators (e.g., inhibitors) of the invention can contain any one, two, three, four or five of the interacting moieties. In specific embodiments the moieties can be small molecules, solvent based molecules, peptides, or peptide mimetics. In related embodiments the moieties fo the invention can be covalently linked. For example, a peptide that binds in the regions defined as A and B can be covalently linked to a small molecule that binds to C. In at least one embodiment, the compounds of this invention do not include fredericamycin A, or compounds related to fredericaniycin A.

In another embodimet the invention pertains to a method for designing an inhibitor of a second peptidyl/prolyl isomerase comprising the steps of:

(a) providing a first peptidyl/prolyl isomerase having a known three-dimensional structure;

(b) identifying amino acids in the acive site of said first peptidyl/prolyl isomerase that form close contacts with a compound know to bind to said active site;

(c) employing protein alignment means to identify in a second peptidyl/prolyl isomerase one or more amino acids that align with, but differ in identity from, said close contact amino acids in said first peptidyl/prolyl isomerase;

(d) altering an amino acid in the active site of said second peptidyl/prolyl isomerase identified in step (c) to produce a mutant second peptidyl/prolyl isomerase;

(e) determining that said compound binds with at least 10-fold greater affinity to said mutant second peptidyl/prolyl isomerase than to said second peptidyl/prolyl isomerase, and

(f) using molecular modeling means to determine how to modify said compound to design an inhibitor of said second peptidyl/prolyl isomerase, wherein said inhibitor binds to said second peptidyl/prolyl isomerase with at least 10-fold greater affinity than said compound binds to said peptidyl/prolyl isomerase, respectively.

In a related embodiment, the first peptidyl/prolyl isomerase is Pin1. In related embodiments the Pin1 molecule can have one or more conservative amino acid substitutions.

In a related embodiment, the second peptidyl/prolyl isomerase is selected from the group consisting of hCyP-A, hCyP-B, hCyP-C, NKCA, hFKBP-12, hFKBP-13, Par14 (Uchida, T. et al. (1999) FEBS Lett. 446, 278-282) and hFKBP-25.

### BRIEF DESCRIPTION OF THE FIGURES

FIG. 1 depicts the amino acid sequence of Pin1 (SEQ ID NO:1), and Pin1 R14A (SEQ ID NO:2).

FIG. 2 is a schematic representation of the defined areas of the polyl isomerase active site of Pin1.

FIG. 3 depicts the structures of compounds used in crystallized co-complexes of Pin1 R14A, and the structures of derivatives of these compounds.

### DETAILED DESCRIPTION

The features and other details of the invention will now be more particularly described and pointed out in the claims. It will be understood that the particular embodiments of the invention are shown by way of illustration and not as limitations of the invention. The principle features of this invention can be employed in various embodiments without departing from the scope of the invention.

The present invention is based, at least in part, on the discovery of a model for designing specific inhibitors of Pin1 and Pin1-related proteins using the structure of Pin1 R14A alone and co-complexed to various inhibitors as determined by X-ray crystallography.

I. Methods of Designing Pin1 Inhibitors

A. Definitions

The term “amino acids” refers to L-isomers of the naturally occurring amino acids or unnatural amino acids. Naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, omithine and lysine.

The term “unnatural amino acids” refers to amino acids that are not naturally found in proteins. Examples of unnatural amino acids as used herein, include racemic mixtures of selenocysteine and selenomethionine. In addition, unnatural amino acids include the D or L forms of nor-leucine, para-nitrophenylalanine, homophenylalanine, para-fluoropheynlalanine, 3-amino-2-benzylpropionic acid, homoarginine, and D-phenylalanine.

The term “positively charged amino acid” refers to any naturally occurring or unnatural amino acid having a positively charged side chain under physiological conditions. Examples of positively charged amino acids include arginine, lysine and histidine.

The term “negatively charged amino acid” refers to any naturally occurring or unnatural amino acid having a negatively charged side chain under normal physiological conditions. Examples of negatively charged amino acids include aspartic acid and glutamic acid.

The terms “hydrophobic amino acid” or “nonpolar amino acid” refer to any amino acid having an uncharged, nonpolar side chain that is relatively insoluble in water. Examples of hydrophobic amino acids include alanine, leucine, isoleucine, valine, proline, phenylalanine, tryptophan and methionine.

The term “hydrophilic amino acid” refers to any amino acid having a uncharged, polar side chain that is relatively soluble in water. Examples of hydrophilic amino acids include serine, threonine, tyrosine, asparagine, glutamine and cysteine.

The terms “protein” and “polypeptide” are used interchangeably herein. The term “peptide” is used herein to refer to a chain of two or more amino acids or amino acid analogs (including non-naturally occurring amino acids), with adjacent amino acids joined by peptide (—NHCO—) bonds. Thus, peptides in accordance with the invention include oligopeptides, polypeptides, proteins, and peptidomimetics.

The term “three dimensional coordinate set” refers to mathematical coordinates derived from mathematical equations related to the patterns obtained on diffraction of a monochromatic beam of X-rays by the atoms of a protein molecule in a crystal form. The diffraction data are used to calculate an electron density map of the repeating unit of the crystal. The electron density maps are used to establish the positions of the individual atoms within the unit cell.

The term “unit cell” refers to the basic parallelepiped shaped block. The entire volume of the crystal may be constructed by regular assembly of such blocks.

The term “space group” refers to the arrangement of symmetry elements of a crystal.

The term “Pin1 polypeptide” refers to a polypeptide having a substitution, insertion or deletion of one of more amino acids of SEQ ID NO:1 wherein the active site of the Pin1 polypeptide is accessible to solvent and available for interaction with an inhibitor in a crystallized form. The term “Pin1 polypeptide” can also refer to any folded, globular domain or fragment of the Pin1 protein of SEQ ID NO:1 that is useful in the design of modulators of the Pin1 polypeptide.

The term “Pin1 related polypeptide” refers to a polypeptide that is homologous or functionally conserved with Pin1 (SEQ ID NO:1).

The term “Pin1-associated state” or “Pin1 associated disorder” includes disorders and states (e.g., a disease state) which are associated with abnormal cell growth, abnormal cell proliferation, or aberrant levels of Pin1 marker. Pin1-associated states include states resulting from an elevation in the expression of cyclin D1 and/or Pin1. Pin1-associated states also include states resulting from an elevation in the phosphorylation level of c-Jun, particularly phosphorylation of c-Jun on Ser^{63/73}-Pro and/or from an elevation in the level of c-Jun amino terminal kinases (JNKs) present in a cell. Pin1-associated states include neoplasia, cancer, undesirable cell growth, and/or tumor growth. Pin1-associated states include states caused by DNA damage, an oncogenic protein (i.e. Ha-Ras), loss of or reduced expression of a tumor suppressor (i.e. Brcal), and/or growth factors.

The term “fragment” refers to any portion of the Pin1 polypeptide of SEQ ID NOs: 1 and 2 which retains all, or any portion, of the structural elements of the native polypeptide.

The term “interacting agent” includes substances which can interact with the Pin1 polypeptide such that the three-dimensional structure can be determined. In one embodiment, the interacting agent is a Pin1 inhibitor, a substrate-derived peptide, or a solvent based molecule.

The term “co-complex” refers to a Pin1 polypeptide, Pin1 related polypeptide or fragment thereof in covalent or non-covalent association with a chemical entity or compound.

The term “Pin1 inhibitor” refers to any molecule that can interact with Pin1 or a Pin1-related polypeptide and inhibit the ability of the polypeptide to carry out proline isomerization activity. In certain embodiments, the inhibitor is specific for Pin 1, i.e., does not inhibit the isomerase activity of PPIases belonging to other classes (e.g., cyclophilins or FKBPs).

A “competitive” inhibitor as used herein is one that inhibits proline isomerase activity by binding to the same kinetic form of the enzyme as its substrate binds, thus directly competing with the substrate for the active site. Competitive inhibition can be reversed completely by increasing the substrate concentration. An “uncompetitive” inhibitor as used herein is one that inhibits proline isomerase activity by binding to a different kinetic form of the enzyme than does the substrate. Such inhibitors bind to the enzyme already bound with the substrate and not to the free enzyme. Uncompetitive inhibition cannot be completely reversed by increasing the substrate concentration. A “noncompetitive” inhibitor as used herein is one that can bind to either the free or substrate bound enzyme.

The term “WW domain” refers to a 30-40 residue domain that is comprised of a three-stranded anti-parallel β sheet and is known to bind proline-rich or proline-containing ligands. In one embodiment, the WW domain is a fragment of Pin1 that binds phosphorylated protein or peptide substrates.

The term “hydrophobic pocket” refers to the portion of the active site that binds a hydrophobic moiety. In one embodiment, the hydrophobic pocket contains 4, 6, 8, 10, 12 or 14 hydrophobic amino acid residues. In one particular embodiment, the hydrophobic pocket contains amino acid residues His59, Leu61, Leu122, Met130, Gln131, Phe134, Thr152, and His157 of SEQ ID NO:1.

The term “cysteine/serine valley” refers to a portion of the active site that is responsible for binding or interacting with the isomerized peptide bond moiety of the substrate. In one embodiment this region contains residues Leu61, Cys113 and Ser154 of SEQ ID NO:1.

The term “phosphate binding pocket” refers to a region of the active site containing three positively charged amino acids that binds or interacts with negatively charged moieties or hydrogen donor/acceptor groups. In one embodiment, this pocket is contains 4, 6, 8, or 10 amino acid residues. In one particular embodiment, this pocket is defined by residues Lys63, Ser67, Arg68, Arg69, Pro70 and Ser154.

The term “substrate entry groove” refers to a region of the polypeptide that allows for substrate entry into the active site. In one embodiment this groove contains amino acids Lys63, Arg69, Ser71, Ser72, Trp73, Arg74, Gln75, Glu76, Asp112, Cys113, Ser114.

The term “lip regions” refers to the residues that surround the active site cavity, as defined previously. In one embodiment these lip regions contain residues that are within 10 Å of the active site cavity. In one particular embodiment, this lip region is defined by, but not limited to, residues Arg54, Arg56, His64, Ser65, Gln66, Lys77, Ile78, Thr79, Ser115, Lys117, Ala118, Gly123, Ala124, Phe125, Ser126, Arg127, Gly128, Gln129, Pro133, Glu135, Lys132, Phe151, Asp153, Gly155, and Ile156.

The term “hydrophobic pocket interacting moiety” refers to a compound that is capable of interacting with the residues of the hydrophobic pocket. This interaction can be, for example, through hydrophobic interactions, through van der Waals contacts, or through hydrogen bonds.

The term “cysteine/serine valley interacting moiety” refers to a compound that is capable of interacting with Cys113 or Ser154, i.e., the cysteine/serine valley, within the region of the active site that binds the serine residue of the natural substrate. This interaction can be covalent, noncovalent, throught hydrogen bonds, or by van der Waals interactions.

The term “phosphate binding pocket interacting moiety” refers to a compound that can interact with the phosphate binding pocket. This interaction can be electrostatic, through salt bridges, covalent, or through van der Waals interactions.

The term “substrate entry groove interacting moiety” refers to a compound that can interact with the substrate entry groove. This interaction can be electrostatic, hydrophobic, covalent, hydrogen bonding, or through van der Waals interactions

The term “lip region interacting moiety” refers to a compound that is capable of interacting with the residues outside the active site. This interaction can be, for example through hydrophobic interactions, though van der Waals contacts, or through hydrogen bonds.

The term “interaction template” refers to a three dimensional model built using information obtained from crystal structures solved with chemical entities bound in the active site. The interaction template is formed by using a Pin1 polypeptide having a substitution insertion or deletion of one or more amino acids of the amino acid sequence set forth in SEQ ID NO:1, wherein the active site of the Pin1 polypeptide is accessible to solvent and available for interaction with a inhibitors. This crystallized form of Pin1 is used to facilitate solving high resolution molecular structures with molecules bound in the active site such that the residues capable of interacting with inhibitors in the active site are determined. The interaction of molecules bound to Pin1 are mapped and characterized by type, distance, and position. The accumulation of interaction information is used to focus the modification of know inhibitors, or to suggest novel design motifs. This template is thus used in the design of high affinity inhibitors of Pin1 isomerase activity.

The term “specificity template” refers to a template created by comparing sequence and structural alignments of homologous or functionally related proteins. Identification of conserved and non-conserved residues and regions allows a skilled artisan to design inhibitors of prolyl isomerase activity that increase affinity and specificity. The determination of conserved amino acids allows the skilled artisan to develop inhibitors with increased affinity but not, necessarily, specificity. The determination of non conserved amino acids and regions allows the skilled artisan to develop inhibitors that have increased affinity and specificity.

B. Crystals of Pin1 R14A and Pin1 R14A-Inhibitor Complexes

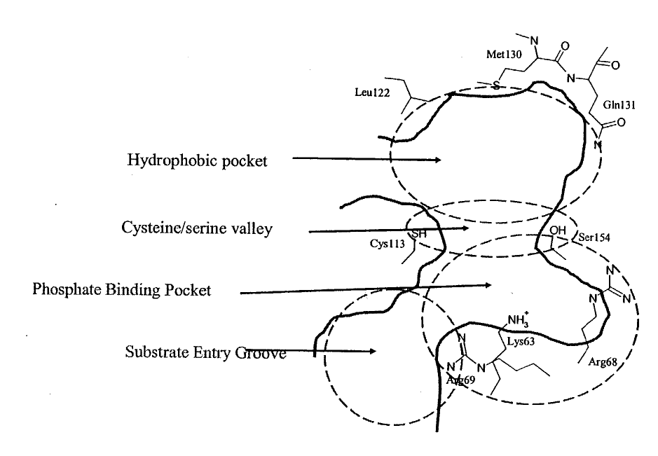

The present invention is based, at least in part, on the discovery that crystals of Pin1 polypeptide containing an alanine at position 14 of the amino acid sequence (Pin1 R14A) exhibit properties that are advantageous over the wild-type polypeptide in the crystallized form. The Arg at position 14 is located in the WW domain (Ranganathan, R. et al. Cell 89:875-86). Mutation of the Arg14 residue to a non-charged amino acid results in an alteration within the WW domain structure, resulting in the crystallization of the Pin1 polypeptide in a new crystallization space group of P3₁21 with unit cell dimensions a=b=68.7 and c=79.4 with α=90′, β=90° and y=120°. The atomic coordinates of Pin1 R14A are listed in FIG. 2.

The Arg14 mutation does not cause any alteration in the structure of the proline isomerase domain, or in the catalytic ability of Pin1. The resulting crystals are mechanically robust, do not require microseeding to improve crystal size, and diffract to a higher resolution than crystals of the wild-type Pin1 polypeptide. Additionally, these crystals grow in a shorter period of time, typically less than 2 days rather than the 1-2 weeks for the wild-type polypeptide.

It was therefore determined that the Pin1 R14A crystals are particularly well suited for inhibitor screening by soaking low affinity and nonspecific inhibitors into the active site of a grown crystal. In addition to the previously stated properties, the peptidyl-prolyl cis-trans isomerase active site is exposed within the crystal lattice to the large solvent channels within the crystal. This allows easy access for soluble compounds to the proline isomerase active site within already formed crystals. It also allows for the soaking of Pin1 R14A crystals in solutions containing potential inhibitors, giving the modulator access to bind to the active site. Crystal soaking does not then require a re-optimization of the crystallization conditions, and any number of inhibitors can be screened, thus increasing the speed at which inhibitor-protein structures may be determined. The robust nature of the crystals also facilitates the manipulation and transfer of the crystals into solutions required for soaking potential inhibitors into existing crystals, and for X-ray data collection.

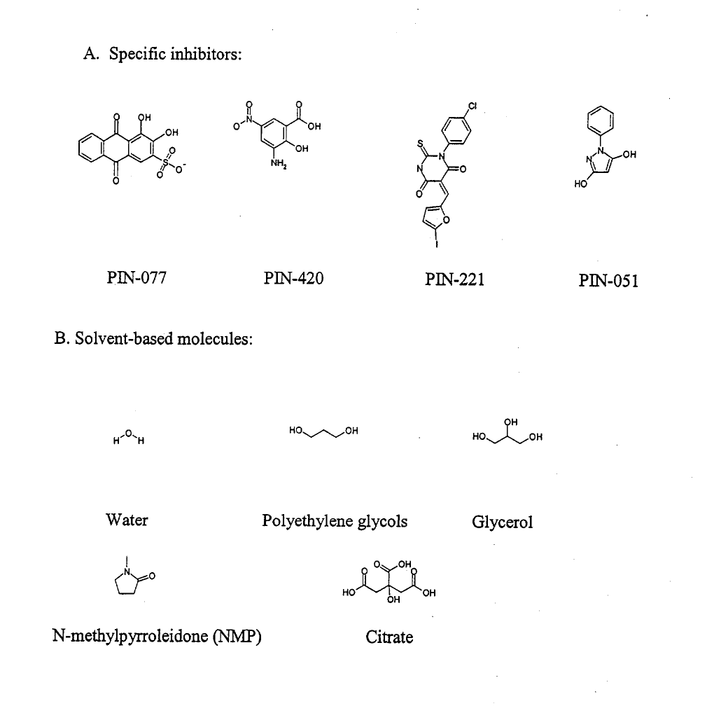

As described in the Examples, crystals of Pin1 R14A associated in a co-complex with various types of inhibitors were also produced and analyzed. The three-dimensional coordinates for crystals of two of the inhibitors, PIN-077 and PIN-420, bound to the active site of Pin1 R14A are depicted in FIGS. 3 and 4, respectively.

Data sets collected of Pin1 crystals with molecules bound in the active site allow for elucidation of the residues capable of interacting with inhibitors. Using these data sets, an interaction template can be built that allows the skilled artisan to design inhibitors specific for the Pin1 active site.

Based on sequence and structural alignments of related enzymes, the phosphate binding pocket has been determined to be a key region for targeting the design for increased interaction and specificity. The crystal structure of the co-complex with inhibitor PIN-077 (the coordinates of which are presented in Table 2) shows that the sulfate moiety of this molecule makes four hydrogen bond contacts in the phosphate binding pocket (with Lys63, Arg69, Ser154, and bound water) and that the hydroxyl groups on the proximal ring hydrogen bond to the nitrogen of the Arg69 sidechain and to Lys63. Investigation of the remainder of the structure demonstrated that the remaining two rings of PIN-077 do not bind in the active site cavity, but rather protrude out of the active site. The structure of citrate bound in the phosphate pocket (the coordinates of which are presented in Table 6), provides another acidic moiety type bound in the phosphate pocket. The citrate makes hydrogen bond contacts with Lys63 and Arg69. Both examples are integrated into the interaction template by providing a preferred interaction pattern, along with unique type, distance, and linking atoms.

Interestingly, the hydrophobic pocket has a role in binding the proline residue of the substrate, and has a variety of motifs which bind into this active site region. Based on the specificity template, this region will be less specific, but will increase binding affinity. In the Pin R14A/Pin-077 crystal structure it was observed that a polyethylene glycol molecule was bound in the hydrophobic pocket showing the formation of a hydrogen bond between the hydroxyl group of the polyethylene glycol molecule and a backbone nitrogen atom (Gln131) of the Pin1 polypeptide. A hydrogen bond between Gln131 backbone nitrogen atom and an oxygen atom on the bound ligand was also observed with inhibitor Pin-420, N-methylpyrroleidone, and glycerol. The preferred placement of an oxygen atom at this position is thus incorporated and emphasized in the interaction template.

The crystal structure of the co-complex with PIN-420 shows that this molecule binds into the hydrophobic pocket, making contact with Gln131 and Met130. PIN-420 also initeracts with residues in the cysteine/serine valley, Cys113 and Ser154. PIN-420 alters the local protein environment by disrupting the hydrogen bond observed in other structures between Gln131 and Ser154. These residues sidechains no longer interact with each other, but rather form hydrogen bonds to PIN-420.

Accordingly, in one aspect the invention features a crystallized Pin1 polypeptide containing an amino acid other than arginine at position 14 of the polypeptide sequence. In certain embodiments, the amino acid at position 14 is also non-polar, and in one preferred embodiment, the amino acid at position 14 is alanine. In related embodiments, the invention pertains to crystals of any part or fragment of a Pin1 polypeptide of the invention that contains the residues that comprise the isomerase active site (e.g., fragments of Pin1 R14A).

In a related aspect, the invention also pertains to crystals of the Pin1 polypeptides containing an amino acid other than arginine at position 14 in a co-complex with an inhibitor of the Pin1 polypeptide. In certain embodiments, the amino acid at position 14 is also a non-polar amino acid, and in one preferred embodiment, the amino acid at position 14 is alanine. In other embodiments, the crystallized co-complexes contain Pin1 R14A or fragments thereof.

The crystallized Pin1 polypeptides and crystallized Pin1 polypeptide/inhibitor co-complexes are obtained by growing the crystals under appropriate conditions such that the three-dimensional structure can be determined using methods described herein and/or art recognized techniques. For example, Pin1 crystals of Pin1 can be grown by a number of techniques including batch crystallization, vapor diffusion (either by sitting drop or hanging drop), or by microdialysis. Once a crystal has been obtained X-ray diffraction data can be collected. Similarly, the crystallized co-complexes of this aspect of the invention can be obtained by growing the crystal in the presence of the inhibitor, or can be obtained by first growing the crystal to the appropriate size, followed by soaking said crystal in a solution that contains an inhibitor.

The term “appropriate conditions” include those conditions which result in the formation of a crystal which can be analyzed to a resolution of 5.0 Å, 4.0 Å, 3.0 Å, 2.0 Å or greater. In one embodiment, the temperature of crystallization of the Pin1 polypeptide is from about 1° C. to about 30° C., from about 1° C. to about 25° C., from about 1° C. to about 15° C., from about 1° C. to 10° C., or about 4° C. In a further embodiment, the conditions are selected such that crystals of the Pin1 polypeptide grow within an acceptable time and reach dimensions that are suitable for structural determination, e.g., by using X-ray diffraction. In certain embodiments, the acceptable time for crystal growth is 1 week or less, 5 days or less, 4 days or less, 3 days or less, or, advantageously, 2 days or less. In related embodiments, the dimensions of the crystal are 0.05 mm or greater per side, 0.1 mm or greater per side, 0.2 mm or greater per side, or approximately 0.3 mm per side or greater. Still other embodiments are presented in the Examples described herein.

C. Designing Inhibitors of Pin1 Based on the Structure Coordinates of Pin1 R14A and Pin1 R14A-Inhibitor Co-Complexes

The crystallized Pin1 R14A and Pin1 R14A-inhibitor complexes have, for the first time, provided structural information that has enabled identification of the regions of the Pin1 polypeptide involved in the molecular interaction with Pin1 inhibitors, thereby providing methods for identifying specific inhibitors of Pin1. By comparison of the crystal structures of Pin1 R14A and the Pin1 R14A-inhibitor complexes described herein, potential sites for modification within the inhibitors have been identified providing an important tool for determining the most efficient binding interactions between Pin1 and a potential inhibitor.

The Pin1 polypeptide consists of two structural domains organized around a hydrophobic cavity. The N-terminal WW domain, defined as residues 1-39 and a C-terminal proline isomerase domain, defined as residues 45-163. The WW domain is a three-stranded anti-parallel β-sheet. The C-terminal proline isomerase domain is a 4 stranded anti-parallel β-sheet surrounded by 4 α helices (e.g., Ranganathan, et al. Cell 89:875-886, 1997; Verdecia et al., Nat Struct Biol 7(8):639-43, 2000).

Based on the comparison of the structural coordinates of the crystals of Pin1 R14A and co-complexes, five regions of the active site involved in key interactions which define Pin1 enzyme activity and specificity were elucidated. As shown in FIG. 9, these regions include the following:

The “hydrophobic pocket” is composed of residues His59, Leu61, Leu122, Met130, Gln131, Phe134, Thr152, His157 of Pin1 (SEQ ID NO.1). This pocket binds the hydrophobic side chain of the substrate proline.

Residues Leu61, Cys113, and Ser154 in the active site are here defined as a “cysteine/serine valley”, residing between the “hydrophobic pocket” and the “phosphate binding pocket”. The isomerized peptide bond of an alanine-proline dipeptide was found to be located in this region.

The third region is the basic “phosphate binding pocket” consisting of a region of high localized positive charge and is defined with amino acids Lys63, Ser67, Arg68, Arg69, Pro70, and Ser154. This region is part of the specificity loop (amino acids 64-80) for the phosphate-specific recognition by Pin1 of phosphorylated serine/threonine peptide substrates which are uniquely recognized and isomerized the Pin1 family of proline isomerases, in contrast to FK506 binding proteins and cyclophilins.

A fourth region is the “substrate entry groove” as defined by the following amino acids: Lys63, Arg69, Ser71, Ser72, Trp73, Arg74, Gln75, Glu76, Asp112, Cys113, Ser114.

A fifth region is the “lip region” as defined by the residues that immediately surround the active site cavity, as defined previously. In one embodiment these lip regions contain residues that are within 10 Å of the active site cavity. In one particular embodiment, this lip region is defined by, but not limited to, residues Arg54, Arg56, His64, Ser65, Gln66, Lys77, Ile78, Thr79, Ser115, Lys117, Ala118, Gly123, Ala124, Phe125, Ser126, Arg127, Gly128, Gln129, Pro133, Glu135, Lys132, Phe151, Asp153, Gly155, and Ile156.

Accordingly, in another aspect, the invention provides a method of designing specific inhibitors of Pin1 isomerase activity based on the ability of molecules to bind to one or more of the defined regions of the Pin1 proline isomerase active site.

In one embodiment, the method uses the structure coordinates of Pin1 R14A and/or Pin1 R14A-inhibitor complexes to design compounds that bind to at least one of the regions of the active site. In certain embodiments, the compounds are designed to bind at least one, preferably to at least two, more preferably at least three, four, or five regions of the active site.

In another embodiment, the method uses a crystal of Pin1R14A probed with molecules composed of a variety of different chemical moieties to determine optimal sites for interaction between candidate Pin1 inhibitors and the enzyme. For example, small molecules that bind tightly to one or more of the regions of the active site can be designed and tested for their ability to inhibit Pin1 isomerase activity using the assays described herein.

Another method included in this aspect of the invention is to computationally screen small molecule databases for chemical entities that can bind in whole, or in part to, to one or more of the regions of the Pin1 active site. In one embodiment of this method, the quality or fit of the compound identified to the regions of the Pin1 active site can be judged either by shape complementarity or by estimated interaction energy (Meng et al., J. Comp. Chem. 13:505-524, 1992).

In a further embodiment, potential inhibitors that can be analyzed according to the methods of the invention can be obtained using any of the numerous approaches in combinatorial library methods known in the art. In this embodiment, potential inhibitors are first identified for Pin1 inhibitory activity using the in vitro assays described herein. Once potential inhibitors are identified, and their structures determined, further optimization can be carried out by computational analyses using the structure coordinates of the Pin1 R14A and Pin1R14A-co-complexes described herein. Alternatively, further optimization can be carried out by determining the structural coordinates of crystallized co-complexes of the potential inhibitor and Pin1 R14A using the methods described herein.

Various combinatorial libraries that can be used in the methods of the invention include, but are not limited to: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Further, examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

The potential inhibitory effect of a compound can be further analyzed prior to its actual synthesis and testing by use of computer modeling techniques using the structural coordinates of the Pin1 R14A and Pin1 R14A-inhibitor co-complexes described herein. If the computer modeling indicates a strong interaction, the molecule can then be synthesized using standard methods known to those skilled in the chemical arts, and then tested for its ability to inhibit Pin1 isomerase activity using the assays set forth herein.

An inhibitory or other binding compound of Pin1 may be computationally evaluated and designed by means of a series of steps in which chemical entities or fragments are screened and selected for their ability to associate with the individual binding pockets or other areas of Pin1.

In other embodiments of the method of the invention, potential inhibitory compounds can be examined for their ability to associate with Pin1 and more particularly with the five regions of the Pin1 active site. This process can involve visual inspection of, for example, the active site on a computer screen based on the coordinates of the Pin1 R14A and Pin1 R14A-inhibitor complex as set forth in Tables 1-6. Selected compounds or chemical moieties can then be positioned in a variety of orientations, or docked, within an individual region of the Pin1 active site as defined herein. Docking can be accomplished using software such as Quanta and SYBYL, followed by energy minimization and molecular dynamics with standard molecular mechanics forcefields, such as CHARMM and AMBER.

Specialized computer programs that can also be used in the process of selecting compounds or chemical entities include:

1. SYBYL Available from Tripos Inc., 1699 South Hanley Rd., St. Louis, Mo., 63144, USA

2. UNITY Available from Tripos Inc., 1699 South Hanley Rd., St. Louis, Mo., 63144, USA

3. FlexX Available from Tripos Inc., 1699 South Hanley Rd., St. Louis, Mo., 63144, USA

4. GRID (Goodford, P. J., “A Computational Procedure for Determining Energetically Favorable Binding Sites on Biologically Important Macromolecules”, J. Med. Chem., 28, pp. 849-857 (1985)). GRID is available from Oxford University, Oxford, UK.

5. MCSS (Miranker, A. and M. Karplus, “Functionality Maps of Binding Sites: A Multiple Copy Simultaneous Search Method.” Proteins: Structure. Function and Genetics, 11, pp. 29-34 (1991)). MCSS is available from Molecular Simulations, Burlington, Mass.

6. AUTODOCK (Goodsell, D. S. and A. J. Olsen, “Automated Docking of Substrates to Proteins by Simulated Annealing”, Proteins: Structure. Function, and Genetics, 8, pp. 195-202 (1990)). AUTODOCK is available from Scripps Research Institute, La Jolla, Calif.

7. DOCK (Kuntz, I. D. et al., “A Geometric Approach to Macromolecule-Ligand Interactions”, J. Mol. Biol., 161, pp. 269-288 (1982)). DOCK is available from University of California, San Francisco, Calif.

Once suitable compounds or chemical moieties have been selected, they can be assembled into a single compound or inhibitor. Assembly may be proceed by visual inspection of the relationship of the compounds or moieties to each other on the three-dimensional image displayed on a computer screen in relation to the structure coordinates of Pin1, Pin1 R14A and/or Pin1 R14A-inhibitor co-complexes. This could then be followed by manual model building using software such as Quanta or SYBYL.

Other useful programs to aid one of skill in the art in connecting the individual compounds or chemical entities include:

1. CAVEAT (Bartlett, P. A. et al, “CAVEAT: A Program to Facilitate the Structure-Derived Design of Biologically Active Molecules”. In “Molecular Recognition in Chemical and Biological Problems”, Special Pub., Royal Chem. Soc., 78, pp. 182-196 (1989)). CAVEAT is available from the University of California, Berkeley, Calif.

2. 3D Database systems such as MACCS-3D (MDL Information Systems, San Leandro, Calif.). This area is reviewed in Martin, Y. C., “3D Database Searching in Drug Design”, J. Med. Chem., 35, pp. 2145-2154 (1992)).

3. HOOK (available from Molecular Simulations, Burlington, Mass.).

In other embodiments, Pin1 inhibitors can be designed as a whole or “de novo” using either an empty active site or optionally including some portion(s) of a known inhibitor(s), e.g., PIN-051 and/or PIN-077, as described herein. Programs which can aid in these methods include:

1. LUDI (Bohm, H.-J., “The Computer Program LUDI: A New Method for the De Novo Design of Enzyme Inhibitors”, J. Comp. Aid. Molec. Design, 6, pp. 61-78 (1992)). LUDI is available from Biosym Technologies, San Diego, Calif.

2. LEGEND (Nishibata, Y. and A. Itai, Tetrahedron, 47, p. 8985 (1991)). LEGEND is available from Molecular Simulations, Burlington, Mass.

3. LeapFrog (available from Tripos Associates, St. Louis, Mo.).

Other molecular modeling techniques may also be employed in accordance with this invention. See, e.g., Cohen, N. C. et al., “Molecular Modeling Software and Methods for Medicinal Chemistry”, J. Med. Chem., 33, pp. 883-894 (1990). See also, Navia, M. A. and M. A. Murcko, “The Use of Structural Information in Drug Design”, Current Opinions in Structural Biology, 2, pp. 202-210 (1992).

Once a compound has been designed or selected by the above methods, the efficiency with which that compound inhibits Pin1 can be tested and optimized by computational evaluation. For example, a compound that has been designed or selected to function as a Pin1-inhibitor must also preferably traverse a volume not overlapping that occupied by the active site when it is bound to the native substrate. An effective Pin1 inhibitor must preferably demonstrate a relatively small difference in energy between its bound and free states (i.e., a small deformation energy of binding).

A compound designed or selected as an inhibitor of Pin1 can be further computationally optimized so that in its bound state it would preferably lack repulsive electrostatic interaction with the target enzyme. Such non-complementary (e.g., electrostatic) interactions include repulsive charge-charge, dipole-dipole and charge-dipole interactions. Specifically, the sum of all electrostatic interactions between the inhibitor and the enzyme when the inhibitor is bound to Pin1 preferably make a neutral or favorable contribution to the enthalpy of binding.

Specific computer software is available in the art to evaluate compound deformation energy and electrostatic interaction. Examples of programs designed for such uses include: Gaussian 92, revision C, M. J. Frisch, Gaussian, Inc., Pittsburgh, Pa.; AMBER, version 4.0, P. A. Kollman, University of California at San Francisco; QUANTA/CHARMM, Molecular Simulations, Inc., Burlington, Mass.; and Insight II/Discover (Biosysm Technologies Inc., San Diego, Calif.). These programs may be implemented, for instance, using a Silicon Graphics workstation, IRIS 4D/35 or IBM RISC/6000 workstation model 550. Other hardware systems and software packages will be known to those skilled in the art.

Once a Pin1 inhibitor has been optimally selected or designed, as described herein, substitutions can then be made in some of its atoms or side groups in order to improve or modify its binding properties, again using the information provided by the interaction and specificity templates to identify regions amiable to modification. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to Pin1 by the same computer methods described in detail, above.

Designed inhibitors can be further evaluated using in vivo or in vitro assays.

D. Assays

Cellular and Cell-Free Assays

Determining the ability of the Pin1 polypeptide to bind to a compound of the invention can also be accomplished by determining direct binding. Determining the ability of the Pin1 protein to bind to a potential inhibitor can be accomplished, for example, by coupling the Pin1 protein with a radioisotope or enzymatic label such that binding of the Pin1 protein to the potential inhibitor can be determined by detecting the labeled Pin1 protein in a complex. For example, Pin1 molecules, e.g., Pin1 proteins, can be labeled with ¹²⁵I, ³⁵S, ¹⁴C, or ³H, either directly or indirectly, and the radioisotope detected by direct counting of radioemmission or by scintillation counting. Alternatively, Pin1 molecules can be enzymatically labeled with, for example, horseradish peroxidase, alkaline phosphatase, or luciferase, and the enzymatic label detected by determination of conversion of an appropriate substrate to product.

In other embodiments, determining the ability of the inhibitor to bind to Pin1 can be determined by detecting induction of a downstream event (e.g., expression of cyclin D1, mitosis etc.), detecting the induction of a reporter gene (comprising a target-responsive regulatory element (e.g. AP-1) operatively linked to a nucleic acid encoding a detectable marker, e.g., chloramphenicol acetyl transferase), or detecting another Pin1-regulated cellular response.

In another embodiment, the assay is a cell-free assay in which a Pin1 protein or biologically active portion thereof is contacted with a test compound and the ability of the test compound to inhibit the activity of the Pin1 protein or biologically active portion thereof is determined. Determining the ability of the test compound to inhibit the activity of a Pin1 protein can be accomplished, for example, by determining the ability of the Pin1 protein to bind to a Pin1 target molecule by one of the methods described above for determining direct binding. Determining the ability of the Pin1 protein to bind to a Pin1 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BLAcore). Changes in the optical phenomenon of surface plasmon resonance (SPR) can be used as an indication of real-time reactions between biological molecules.

In an alternative embodiment, determining the ability of the test compound to inhibit the activity of a Pin1 protein can be accomplished by determining the ability of the Pin1 protein to further modulate the isomerization of the activity of a Pin1 target molecule (e.g., a Pin1 substrate, a phosphoprotein). For example, the activity of the effector molecule on an appropriate target can be determined, or the binding of the effector to an appropriate target can be determined as previously described.

In yet another embodiment, the cell-free assay involves contacting a Pin1 protein or biologically active portion thereof with a known compound which binds the Pin1 protein to form an assay mixture, contacting the assay mixture with a test compound, and determining the ability of the test compound to interact with the Pin1 protein, wherein determining the ability of the test compound to interact with the Pin1 protein comprises determining the ability of the Pin1 protein to preferentially bind to or modulate the activity of a Pin1 target molecule.

In more than one embodiment of the above assay methods of the present invention, it may be desirable to immobilize either Pin1 or its target molecule to facilitate separation of complexed from uncomplexed forms of one or both of the proteins, as well as to accommodate automation of the assay. Binding of a test compound to a Pin1 protein, or interaction of a Pin1 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/Pin1 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or Pin1 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of Pin1 binding or activity determined using standard techniques.

Other techniques for immobilizing proteins on matrices can also be used in the assays of the invention. For example, either a Pin1 protein or a Pin1 target molecule can be immobilized utilizing conjugation of biotin and streptavidin. Biotinylated Pin1 protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with Pin1 protein or target molecules but which do not interfere with binding of the Pin1 protein to its target molecule can be derivatized to the wells of the plate, and unbound target or Pin1 protein trapped in the wells by antibody conjugation. Methods for detecting such complexes, in addition to those described above for the GST-immobilized complexes, include immunodetection of complexes using antibodies reactive with the Pin1 protein or target molecule, as well as enzyme-linked assays which rely on detecting an enzymatic activity associated with the Pin1 protein or target molecule.

Tumor Inhibition Assays

In vitro anti-tumor activity of the Pin1-inhibitor compounds of the invention can be assayed by measuring the ability of the compound to kill tumor cells. Examples of appropriate cell lines include: human lung (A549); resistant human lung with low topo II activity (A549-VP); murine melanoma (B16); human colon tumor (HCT116); human clone tumor with elevated p170 levels (HCTVM); human colon tumor with low topo II activity (HCTVP); P388 murine lymph leukemia cells; and human colon carcinoma cell line (Moser)

Tumor inhibition assays are described, for example, in Kelly, et al., U.S. Pat. No. No. 5,166,208, and in Pandley, et al., J. Antibiot. 3(11):1389-401 (1981). In one assay, the cells are allowed to grow for a 24 hour period under standard conditions. After the cells are allowed to attach to the plate for 24 hours (e.g., a 96-well flat bottom plate), the cells are incubated for 72 hours with serially diluted concentrations of the Pin1 inhibitor compound. From these data, the concentration of the compound at which 50% of the cells are killed or growth inhibited (IC₅₀) is determined.

In vivo anti-tumor activity of Pin1 inhibitor compounds of the invention can be assayed by a reduction of tumor cells in mammals (e.g., mice) and a resulting increase in survival time compared to untreated tumor bearing animals. For example, CDF₁ mice are injected interperitoneally with a suspension of P388 murine lymph leukemia cells, Ehrlich carcinoma cells, B16 melanoma cells, or Meth-A fibrosarcoma cells. Some of the injected mice are then treated interperitoneally with a Pin1 inhibitor compound of the invention, and other mice are treated with saline. The in vivo activity of the compound is then determined in terms of the % T/C which is the ratio of the mean survival time of the treated group to the mean survival time of the saline treated group times 100. Yokoi, et al., U.S. Pat. No. 4,584,377; Kelly, et al., U.S. Pat. No. 5,155,208; Warnick-Pickle, et al., J. Antibiot. 34(11):1402-7 (1981); and Pandley et al., supra.

The in vivo anti-tumor activity of the Pin1 inhibitor compounds of the invention can also be assayed against an ovarian tumor growing in a human tumor cloning system as described, for example, in Tebbe, et al., J. Am. Chem. Soc. 93:3793-3795 (1971).

Specificity Assays for Pin1 Inhibitors

The specificity of the Pin1 inhibitor compounds of the invention can be determined by the protease-coupled PPIase assay developed by Fischer et al. (Biomed. Biochim. Acta, 1984, 43: 1101-1111). For example, the enzyme activity of Pin1 can be compared to members of the other known classes of PPIases, cyclophilins (e.g., hCyp18, hCyP-A, hCyP-B, hCyP-C, and NKCA) and FKBPs (e.g., hFKBP12, hFKBP-12, hFKBP-13, and hFKBP-25) in the presence and absence of the compound.

In one assay, hPin1 activity measurements are determined using bovine trypsin (final concentration 0.21 mg/mL, Sigma) as an isomer specific protease and Ac-Ala-Ala-Ser(P)-Pro-Arg-pNA (Jerini, Germany) as a substrate. PPIase activity of hFKBP12 (Sigma) and hCyp 18 (Sigma) is determined with the peptide substrate Suc-Ala-Phe-Pro-Phe-pNA (B achem) and the protease α-chymotrypsin (final concentration 0.41 mg/mL, Sigma). The test can be performed by observing the released 4-nitroanilide at 390 nm with a Hewlett-Packard 8453 UV-vis spectrophotometer at 110° C. The total reaction volume is adjusted to 1.23 mL by mixing appropriate volumes of 35 mM HEPES (pH 7.8) with enzyme and effector solutions. The Pin1 inhibitor compound is freshly diluted from a 1 mg/mL stock solution in DMSO, and pre-incubated at varying concentrations with the enzyme for 5 min (10° C.). Prior to the start of reaction by addition of the respective protease, 2 μL of the peptide substrate stock solution (10 mg/mL in DMSO) is added. The amount of organic solvent is kept constant within each experiment (<0.1%). The pseudo-first-order rate constant k_{obs} for cis/trans isomerization in the presence of PPIase and the first-order rate constant k₀ of the uncatalyzed cis/trans isomerization can be calculated using the Kinetics Software of Hewlett-Packard as well as SigmaPlot2000 for Windows 6.0 (SPSS). The Kᵢ value for inhibition of Pin1 PPIase activity by a Pin1 inhibitor compound of the invention at constant concentrations of substrate ([S₀]<<K_{M}) can then be calculated by fitting the data according to the equation for a competitive “tight-binding” inhibitor using SigmaPlot2000.

II. Methods of Designing Inhibitors of Pin1 Related Molecules

In another aspect, the present invention pertains to methods of designing inhibitors of Pin1-related molecules. In one embodiment of this aspect, the invention provides a method for designing Pin1-like proline isomerase inhibitors. The method comprises one or more of the following steps:

1. Development of a three-dimensional interaction template for ligand binding to the five regions of the active site as described above;

2. Sequence and structural alignments to determine sequence conservation and divergence among Pin1-like protein domains and other proline-isomerases to identify amino acids unique and essential to the specificity of Pin1-like proline isomerases, thereby creating a specificity template;

3. Design specific inhibitors of Pin1 proline isomerases using the interaction template and the specificity template;

4. Confirmation of increased affinity and specificity of designed compounds through biochemical assay; and

5. Conformation of interaction of designed compounds through crystallographic analysis.

In one embodiment, the Pin1-like polypeptide comprises one or more conservative mutations as compared to the wild type protein. These mutations can be at any point in the polypeptide that will not alter the function of Pin1. In certain embodiments, the mutation is a hydrophobic substitution at residue 14, and in one particular embodiment, the mutation is a substitution of alanine at position 14 of the Pin1 amino acid sequence of SEQ ID No:1.

In another embodiment, the Pin1-like polypeptide is a Pin1 homologue. The designed inhibitor should bind tightly to the isomerase and significantly inhibit the activity of the isomerase. In a preferred embodiment, the first isomerase is a peptidyl prolyl isomerase. Even more preferred is that the first isomerase be Pin1 having the amino acid sequence set forth in SEQ ID NO:1.

In certain embodiments the inhibitors have a Kᵢ for Pin1 of less than 0.2 mM, less than 0.1 mM, less than 750 uM, less than 500 uM, less than 250 uM, less than 100 uM, less than 50 uM, less than 500 nM, less than 250 nM, less than 50 nM, less than 10 nM, less than 5 nM, less than 3 nM, less than 1 nM, or less than 0.5 nM.

In related embodiments the Pin1 inhibitor of the invention is intended to include inhibitors that have a Kᵢ or K_{d} that is at least 2, 3, 4, 5, 10, 15, or 20 times less than the Kᵢ or K_{d} for other peptidyl prolyl isomerases, e.g., hCyP-A, hCyP-B, hCyP-C, NKCA, hFKBP-12, hFKBP-13, and hFKBP-25.

In another embodiment, the Pin1-like polypeptide includes cylophilins (e.g., hCyP-A, hCyP-B, hCyP-C, and NKCA) and other FKBPs (e.g., hFKBP-12, hFKBP-13, and hFKBP-25). Sequence alignments of Pin1 and Pin1-like proteins can be carried out to identify amino acids unique and essential to the specificity of the individual proline isomerase active site.

Compounds of the Invention

Pin1 inhibitors of the invention include molecules that interact with one or more regions of the proline isomerase active site through covalent or non-covalent interactions. In certain embodiments, the inhibitor will contain moieties that interact with one or more regions of the active site wherein the moieties are covalently linked. In other embodiments the inhibitor can consist of one or more molecules, each of which interact with a different region of the active site. In preferred embodiments, the inhibitors interact with at least one, preferably at least two, more preferably at least three, and most preferably four regions of the Pin1 active site.

In one particular embodiment, the Pin1 inhibitor comprises some or all: (1) a hydrophobic portion that localizes to the hydrophobic pocket of the Pin1 polypeptide; (2) the placement of a peptide-like like motif in the cysteine/serine valley; (3) a negatively charged or uncharged moiety that interacts with the basic pocket through charge-charge interactions, hydrogen-bond interactions or favorable steric interactions; (4) the ability to replace water molecules in contact with the active site regions; and (5) interactions outside the active site, gaining interaction with the lip regions.

In certain embodiments, the Pin1 inhibitors have the structure:

A-B-C-D-E

wherein A is a hydrophobic pocket binding moiety, B is a cysteine/serine valley binding moiety, C is phosphate pocket binding moiety, D is a substrate groove interacting moiety, and E is a lip region interacting moiety.

The invention is further illustrated by the following examples, which should not be construed as further limiting. The contents of all references, pending patent applications and published patents, cited throughout this application are hereby expressly incorporated by reference. The animal models used throughout the Examples are accepted animal models and the demonstration of efficacy in these animal models is predictive of efficacy in humans.

### EXAMPLES

### Example 1

### Crystallization and Structural Determination of Pin1 R14A

Production of Pin1 R14A:

An N-terminal histidine tagged fusion protein of the Pin1 R14A mutant is expressed in E. coli. The bacteria is grown using Terrific Broth at 37° C. The expression vector (a pET28a derivative) contains Kanamycin resistance, and the protein is induced by lowering the temperature to 20° C. and adding IPTG to 50 μM. After 6-8 hours the bacteria is harvested by centrifugation and stored at −80° C.

Purification of Pin1 R14A:

The purification of Pin1 R14A is based on the modified method of Ranganathan et al. (Cell 1997, 89, 875-886). The E. coli pellet is thawed and suspended at 5 mL per gram in 50 mM Tris (pH 8.0), 500 mM NaCl, 20 mM Imidazole (pH 8.0), 10% glycerol, 1% Tween 20, and 25 mM β-mercaptoethanol. Lysozyme (hen egg white) is added at 0.5 mg/mL and the solution stirred for 30 min at room temperature. The cells were disrupted by sonication, and the solution clearified by centrifugation. The extract was then applied to a 5 mL column of Qiagen Ni-NTA resin at a 1-2 mL/min flow rate. The column was washed with three column volumes of the above buffer, and then bound protein was eluted from the resin with 50 mM Tris (pH 8.0), 500 mM NaCl, 250 mM Imidazole (pH 8.0), and 10% glycerol. Fractions containing protein were pooled and thrombin was added at 0.5 μL/mL of 1 U/μL. The material was placed in a dialysis bag and dialyzed overnight against 50 mM Tris (pH 8.0), 500 mM NaCl, and 25 mM β-mercaptoethanol. The material is then passed through a 0.5 mL column of Qiagen Ni-NTA resin at 1 mL/min, and 0.25 mL column of Benzamidine-Sepharose at 1 mL/min. After concentration, the material was loaded onto a FPLC size-exclusion column (Superdex 75, HiLoad 16/60, Pharmacia). Fractions containing Pin1 R14A were concentrated and dialyzed against 10 mM HEPES (pH 7.5), 20 mM NaCl, 1 mM DTT. After dialysis, the material was concentrated to 15-20 mg/mL. Material not used immediately was stored at −80° C. Milligram quantities of purified Pin1 R14A are obtained from a liter of bacterial culture.

Crystallization of Pin1 R14A:

The protein is crystallized by screening against 1.8-2.05 M Ammonium sulfate, 1% PEG 400, 0.1 M HEPES (pH 7.5), 1 mM DTT. Crystallization is performed using the hanging drop method in a Linbro style plate, with 1 mL solution in the reservoir. Siliconized glass cover slips are inverted over the reservoir after mixing 2 μL protein solution and 2 μL reservoir solution. Trays are wrapped in foil and placed at 4° C. Crystal growth is observable within two days. Crystal growth was shortened to 2-3 days rather than 1-2 weeks for the wild-type protein, and crystal seeding techniques were not required to obtain sufficiently large crystals.

Crystals can be grown from 0.9-1.2 M Sodium citrate, 1% PEG 400, 0.1 M HEPES (pH. 7.5), 5 mM DTT, with 0-5% glycerol.

Transfer of Pin1 R14A Crystals to Cryoprotectant:

The crystals are transferred directly into cryoprotection solutions (i) 30% PEG 400, 100 mM HEPES (pH 7.5), 1 mM DTT, or (ii) 25% MPD, 100 mM HEPES (pH 7.5) or (iii) 30% PEP, 100 mM HEPES (pH 7.5). Crystal transfer to the cryoprotection solution is necessary to preserve the crystal during cooling to −180° C. for data collection, and also to allow compounds to bind under low salt conditions, which is important to increase the solubility of the compounds and to remove the sulfate ion bound in the active site (placed by crystallization conditions, but removable upon soaking for several days).

Data Collection and Processing:

Data collection on a rotating anode X-ray generator is typically 6 hours, using 1 degree of oscillation and 5 minute exposure times. An oscillation range of 60 degrees is required for a complete data set. The conversion of Arg14 to Ala is located in WW domain of Pin1. This mutation results in an altered and favorable crystallization space group of P3₁21. The crystals are mechanically robust and diffract to high resolution (˜1.9 Å). The images were processed and scaled using Denzo and Scalepack (Z. Otwinowski et al., Meth. Enzymol., 1996, 276, 307-326). Molecular replacement (using PDB entry IPIN as the original search model) and refinement was done with CNX (A. Brunger et al., Acta Cryst. (1990), D54, 905-921), model building was done with 0 (Jones, T. A., Zou, J. Y. Cowan, S. W. & Kjelgaard, M. Acta Cryst. A 47, 110-119 (1991)). The set of atomic coordinates of Pin1 R14A as determined by X-ray crystallography is provided as Table 1.

TABLE 1
Set of atomic coordinates of Pin1 R14A as determined by X-ray crystallography

| | | | | | | | | | | | |
|---|---|---|---|---|---|---|---|---|---|---|---|
| ATOM | 1 | CB | LYS | | 6 | 26.317 | 49.386 | −27.408 | 1.00 | 75.75 | C |
| ATOM | 2 | CG | LYS | | 6 | 25.999 | 49.158 | −25.938 | 1.00 | 76.44 | C |
| ATOM | 3 | CD | LYS | | 6 | 26.255 | 50.416 | −25.125 | 1.00 | 77.48 | C |
| ATOM | 4 | CE | LYS | | 6 | 25.985 | 50.186 | −23.650 | 1.00 | 77.75 | C |
| ATOM | 5 | NZ | LYS | | 6 | 26.269 | 51.405 | −22.845 | 1.00 | 78.45 | N |
| ATOM | 6 | C | LYS | | 6 | 24.620 | 47.817 | −28.371 | 1.00 | 74.39 | C |
| ATOM | 7 | O | LYS | | 6 | 23.834 | 48.207 | −27.507 | 1.00 | 74.98 | O |
| ATOM | 8 | N | LYS | | 6 | 26.644 | 48.422 | −29.663 | 1.00 | 75.06 | N |
| ATOM | 9 | CA | LYS | | 6 | 26.106 | 48.159 | −28.298 | 1.00 | 75.05 | C |
| ATOM | 10 | N | LEU | | 7 | 24.249 | 47.086 | −29.418 | 1.00 | 73.33 | N |
| ATOM | 11 | CA | LEU | | 7 | 22.871 | 46.659 | −29.644 | 1.00 | 71.57 | C |
| ATOM | 12 | CB | LEU | | 7 | 22.295 | 46.010 | −28.379 | 1.00 | 71.92 | C |
| ATOM | 13 | CG | LEU | | 7 | 22.952 | 44.689 | −27.979 | 1.00 | 71.84 | C |
| ATOM | 14 | CD1 | LEU | | 7 | 22.289 | 44.121 | −26.733 | 1.00 | 71.98 | C |
| ATOM | 15 | CD2 | LEU | | 7 | 22.841 | 43.718 | −29.142 | 1.00 | 71.81 | C |
| ATOM | 16 | C | LEU | | 7 | 21.935 | 47.763 | −30.123 | 1.00 | 69.78 | C |
| ATOM | 17 | O | LEU | | 7 | 21.756 | 48.780 | −29.454 | 1.00 | 69.52 | O |
| ATOM | 18 | N | PRO | | 8 | 21.327 | 47.567 | −31.303 | 1.00 | 68.04 | N |
| ATOM | 19 | CD | PRO | | 8 | 21.626 | 46.449 | −32.214 | 1.00 | 67.76 | C |
| ATOM | 20 | CA | PRO | | 8 | 20.391 | 48.502 | −31.933 | 1.00 | 66.26 | C |
| ATOM | 21 | CB | PRO | | 8 | 20.024 | 47.787 | −33.233 | 1.00 | 66.72 | C |
| ATOM | 22 | CG | PRO | | 8 | 21.263 | 47.031 | −33.555 | 1.00 | 67.32 | C |
| ATOM | 23 | C | PRO | | 8 | 19.169 | 48.756 | −31.045 | 1.00 | 64.59 | C |
| ATOM | 24 | O | PRO | | 8 | 19.171 | 48.411 | −29.861 | 1.00 | 64.16 | O |
| ATOM | 25 | N | PRO | | 9 | 18.114 | 49.374 | −31.605 | 1.00 | 62.73 | N |
| ATOM | 26 | CD | PRO | | 9 | 18.120 | 50.167 | −32.846 | 1.00 | 63.09 | C |
| ATOM | 27 | CA | PRO | | 9 | 16.901 | 49.656 | −30.830 | 1.00 | 59.43 | C |
| ATOM | 28 | CB | PRO | | 9 | 16.207 | 50.746 | −31.652 | 1.00 | 61.30 | C |
| ATOM | 29 | CG | PRO | | 9 | 17.323 | 51.369 | −32.440 | 1.00 | 63.30 | C |
| ATOM | 30 | C | PRO | | 9 | 15.999 | 48.438 | −30.656 | 1.00 | 54.44 | C |
| ATOM | 31 | O | PRO | | 9 | 15.606 | 47.803 | −31.636 | 1.00 | 54.75 | O |
| ATOM | 32 | N | GLY | | 10 | 15.673 | 48.114 | −29.408 | 1.00 | 48.14 | N |
| ATOM | 33 | CA | GLY | | 10 | 14.791 | 46.987 | −29.158 | 1.00 | 41.85 | C |
| ATOM | 34 | C | GLY | | 10 | 15.451 | 45.633 | −28.957 | 1.00 | 36.53 | C |
| ATOM | 35 | O | GLY | | 10 | 14.763 | 44.648 | −28.689 | 1.00 | 33.03 | O |
| ATOM | 36 | N | TRP | | 11 | 16.773 | 45.579 | −29.077 | 1.00 | 34.92 | N |
| ATOM | 37 | CA | TRP | | 11 | 17.505 | 44.327 | −28.904 | 1.00 | 33.37 | C |
| ATOM | 38 | CB | TRP | | 11 | 18.664 | 44.241 | −29.898 | 1.00 | 31.56 | C |
| ATOM | 39 | CG | TRP | | 11 | 18.240 | 43.993 | −31.302 | 1.00 | 30.03 | C |
| ATOM | 40 | CD2 | TRP | | 11 | 17.911 | 42.727 | −31.885 | 1.00 | 29.07 | C |
| ATOM | 41 | CE2 | TRP | | 11 | 17.564 | 42.967 | −33.235 | 1.00 | 28.10 | C |
| ATOM | 42 | CE3 | TRP | | 11 | 17.875 | 41.411 | −31.399 | 1.00 | 26.09 | C |
| ATOM | 43 | CD1 | TRP | | 11 | 18.082 | 44.923 | −32.292 | 1.00 | 30.05 | C |
| ATOM | 44 | NE1 | TRP | | 11 | 17.678 | 44.315 | −33.453 | 1.00 | 29.35 | N |
| ATOM | 45 | CZ2 | TRP | | 11 | 17.187 | 41.941 | −34.105 | 1.00 | 26.58 | C |
| ATOM | 46 | CZ3 | TRP | | 11 | 17.500 | 40.391 | −32.264 | 1.00 | 25.29 | C |
| ATOM | 47 | CH2 | TRP | | 11 | 17.161 | 40.663 | −33.604 | 1.00 | 26.24 | C |
| ATOM | 48 | C | TRP | | 11 | 18.060 | 44.130 | −27.499 | 1.00 | 33.25 | C |
| ATOM | 49 | O | TRP | | 11 | 18.412 | 45.089 | −26.811 | 1.00 | 32.43 | O |
| ATOM | 50 | N | GLU | | 12 | 18.137 | 42.871 | −27.078 | 1.00 | 31.52 | N |
| ATOM | 51 | CA | GLU | | 12 | 18.677 | 42.542 | −25.769 | 1.00 | 30.58 | C |
| ATOM | 52 | CB | GLU | | 12 | 17.559 | 42.419 | −24.734 | 1.00 | 30.62 | C |
| ATOM | 53 | CG | GLU | | 12 | 16.673 | 41.201 | −24.889 | 1.00 | 32.61 | C |
| ATOM | 54 | CD | GLU | | 12 | 15.547 | 41.189 | −23.878 | 1.00 | 34.53 | C |
| ATOM | 55 | OE1 | GLU | | 12 | 14.612 | 42.008 | −24.013 | 1.00 | 33.83 | O |
| ATOM | 56 | OE2 | GLU | | 12 | 15.606 | 40.372 | −22.937 | 1.00 | 34.83 | O |
| ATOM | 57 | C | GLU | | 12 | 19.437 | 41.229 | −25.880 | 1.00 | 29.52 | C |
| ATOM | 58 | O | GLU | | 12 | 19.170 | 40.417 | −26.768 | 1.00 | 27.64 | O |
| ATOM | 59 | N | LYS | | 13 | 20.392 | 41.031 | −24.985 | 1.00 | 28.84 | N |
| ATOM | 60 | CA | LYS | | 13 | 21.183 | 39.811 | −24.993 | 1.00 | 30.55 | C |
| ATOM | 61 | CB | LYS | | 13 | 22.525 | 40.053 | −24.299 | 1.00 | 32.86 | C |
| ATOM | 62 | CG | LYS | | 13 | 23.505 | 38.895 | −24.393 | 1.00 | 36.09 | C |
| ATOM | 63 | CD | LYS | | 13 | 24.783 | 39.201 | −23.614 | 1.00 | 38.49 | C |
| ATOM | 64 | CE | LYS | | 13 | 25.499 | 40.428 | −24.175 | 1.00 | 39.37 | C |
| ATOM | 65 | NZ | LYS | | 13 | 26.694 | 40.807 | −23.364 | 1.00 | 40.68 | N |
| ATOM | 66 | C | LYS | | 13 | 20.394 | 38.745 | −24.257 | 1.00 | 30.06 | C |
| ATOM | 67 | O | LYS | | 13 | 19.679 | 39.040 | −23.294 | 1.00 | 28.85 | O |
| ATOM | 68 | N | ALA | | 14 | 20.510 | 37.504 | −24.714 | 1.00 | 29.08 | N |
| ATOM | 69 | CA | ALA | | 14 | 19.798 | 36.413 | −24.073 | 1.00 | 29.26 | C |
| ATOM | 70 | CB | ALA | | 14 | 18.550 | 36.049 | −24.882 | 1.00 | 29.89 | C |
| ATOM | 71 | C | ALA | | 14 | 20.705 | 35.199 | −23.915 | 1.00 | 28.49 | C |
| ATOM | 72 | O | ALA | | 14 | 21.813 | 35.161 | −24.448 | 1.00 | 26.85 | O |
| ATOM | 73 | N | MET | | 15 | 20.220 | 34.208 | −23.183 | 1.00 | 29.72 | N |
| ATOM | 74 | CA | MET | | 15 | 20.984 | 32.995 | −22.943 | 1.00 | 29.88 | C |
| ATOM | 75 | CB | MET | | 15 | 21.318 | 32.895 | −21.456 | 1.00 | 33.30 | C |
| ATOM | 76 | CG | MET | | 15 | 22.022 | 31.619 | −21.070 | 1.00 | 36.15 | C |
| ATOM | 77 | SD | MET | | 15 | 23.597 | 31.529 | −21.892 | 1.00 | 39.84 | S |
| ATOM | 78 | CE | MET | | 15 | 24.542 | 32.702 | −20.899 | 1.00 | 37.57 | C |
| ATOM | 79 | C | MET | | 15 | 20.180 | 31.775 | −23.362 | 1.00 | 29.22 | C |
| ATOM | 80 | O | MET | | 15 | 19.071 | 31.573 | −22.883 | 1.00 | 27.04 | O |
| ATOM | 81 | N | SER | | 16 | 20.738 | 30.963 | −24.254 | 1.00 | 28.77 | N |
| ATOM | 82 | CA | SER | | 16 | 20.048 | 29.757 | −24.693 | 1.00 | 28.24 | C |
| ATOM | 83 | CB | SER | | 16 | 20.804 | 29.085 | −25.834 | 1.00 | 28.93 | C |
| ATOM | 84 | OG | SER | | 16 | 20.279 | 27.788 | −26.061 | 1.00 | 30.01 | O |
| ATOM | 85 | C | SER | | 16 | 19.932 | 28.776 | −23.532 | 1.00 | 28.89 | C |
| ATOM | 86 | O | SER | | 16 | 20.932 | 28.418 | −22.910 | 1.00 | 27.90 | O |
| ATOM | 87 | N | ARG | | 17 | 18.706 | 28.352 | −23.244 | 1.00 | 29.91 | N |
| ATOM | 88 | CA | ARG | | 17 | 18.444 | 27.403 | −22.166 | 1.00 | 32.62 | C |
| ATOM | 89 | CB | ARG | | 17 | 16.939 | 27.317 | −21.895 | 1.00 | 34.74 | C |
| ATOM | 90 | CG | ARG | | 17 | 16.323 | 28.597 | −21.357 | 1.00 | 37.67 | C |
| ATOM | 91 | CD | ARG | | 17 | 16.886 | 28.960 | −19.994 | 1.00 | 38.10 | C |
| ATOM | 92 | NE | ARG | | 17 | 16.386 | 30.253 | −19.533 | 1.00 | 40.92 | N |
| ATOM | 93 | CZ | ARG | | 17 | 15.132 | 30.484 | −19.153 | 1.00 | 41.40 | C |
| ATOM | 94 | NH1 | ARG | | 17 | 14.235 | 29.508 | −19.167 | 1.00 | 41.19 | N |
| ATOM | 95 | NH2 | ARG | | 17 | 14.773 | 31.700 | −18.769 | 1.00 | 41.62 | N |
| ATOM | 96 | C | ARG | | 17 | 18.963 | 26.011 | −22.511 | 1.00 | 33.46 | C |
| ATOM | 97 | O | ARG | | 17 | 19.246 | 25.207 | −21.621 | 1.00 | 32.53 | O |
| ATOM | 98 | N | SER | | 18 | 19.086 | 25.730 | −23.804 | 1.00 | 34.96 | N |
| ATOM | 99 | CA | SER | | 18 | 19.552 | 24.424 | −24.259 | 1.00 | 37.48 | C |
| ATOM | 100 | CB | SER | | 18 | 18.902 | 24.070 | −25.599 | 1.00 | 38.32 | C |
| ATOM | 101 | OG | SER | | 18 | 17.506 | 23.886 | −25.458 | 1.00 | 43.49 | O |
| ATOM | 102 | C | SER | | 18 | 21.061 | 24.307 | −24.404 | 1.00 | 37.45 | C |
| ATOM | 103 | O | SER | | 18 | 21.662 | 23.349 | −23.920 | 1.00 | 40.02 | O |
| ATOM | 104 | N | SER | | 19 | 21.669 | 25.286 | −25.066 | 1.00 | 36.22 | N |
| ATOM | 105 | CA | SER | | 19 | 23.102 | 25.271 | −25.311 | 1.00 | 35.77 | C |
| ATOM | 106 | CB | SER | | 19 | 23.377 | 25.684 | −26.759 | 1.00 | 38.18 | C |
| ATOM | 107 | OG | SER | | 19 | 22.862 | 26.978 | −27.025 | 1.00 | 39.68 | O |
| ATOM | 108 | C | SER | | 19 | 23.938 | 26.137 | −24.386 | 1.00 | 35.38 | C |
| ATOM | 109 | O | SER | | 19 | 25.120 | 25.870 | −24.188 | 1.00 | 35.14 | O |
| ATOM | 110 | N | GLY | | 20 | 23.332 | 27.177 | −23.825 | 1.00 | 35.10 | N |
| ATOM | 111 | CA | GLY | | 20 | 24.074 | 28.060 | −22.947 | 1.00 | 33.07 | C |
| ATOM | 112 | C | GLY | | 20 | 24.807 | 29.116 | −23.756 | 1.00 | 31.89 | C |
| ATOM | 113 | O | GLY | | 20 | 25.558 | 29.923 | −23.215 | 1.00 | 31.35 | O |
| ATOM | 114 | N | ARG | | 21 | 24.590 | 29.112 | −25.064 | 1.00 | 30.69 | N |
| ATOM | 115 | CA | ARG | | 21 | 25.241 | 30.083 | −25.929 | 1.00 | 30.10 | C |
| ATOM | 116 | CB | ARG | | 21 | 25.371 | 29.524 | −27.343 | 1.00 | 33.05 | C |
| ATOM | 117 | CG | ARG | | 21 | 26.334 | 30.296 | −28.229 | 1.00 | 37.69 | C |
| ATOM | 118 | CD | ARG | | 21 | 26.025 | 30.027 | −29.687 | 1.00 | 40.08 | C |
| ATOM | 119 | NE | ARG | | 21 | 25.742 | 28.615 | −29.899 | 1.00 | 40.15 | N |
| ATOM | 120 | CZ | ARG | | 21 | 25.137 | 28.125 | −30.973 | 1.00 | 40.11 | C |
| ATOM | 121 | NH1 | ARG | | 21 | 24.745 | 28.932 | −31.951 | 1.00 | 38.33 | N |
| ATOM | 122 | NH2 | ARG | | 21 | 24.915 | 26.821 | −31.059 | 1.00 | 42.15 | N |
| ATOM | 123 | C | ARG | | 21 | 24.403 | 31.360 | −25.966 | 1.00 | 27.27 | C |
| ATOM | 124 | O | ARG | | 21 | 23.173 | 31.308 | −25.940 | 1.00 | 24.40 | O |
| ATOM | 125 | N | VAL | | 22 | 25.071 | 32.502 | −26.033 | 1.00 | 26.38 | N |
| ATOM | 126 | CA | VAL | | 22 | 24.364 | 33.773 | −26.072 | 1.00 | 25.85 | C |
| ATOM | 127 | CB | VAL | | 22 | 25.328 | 34.963 | −25.832 | 1.00 | 28.19 | C |
| ATOM | 128 | CG1 | VAL | | 22 | 26.054 | 34.787 | −24.507 | 1.00 | 31.21 | C |
| ATOM | 129 | CG2 | VAL | | 22 | 26.329 | 35.067 | −26.972 | 1.00 | 29.97 | C |
| ATOM | 130 | C | VAL | | 22 | 23.679 | 33.968 | −27.422 | 1.00 | 23.85 | C |
| ATOM | 131 | O | VAL | | 22 | 24.131 | 33.456 | −28.443 | 1.00 | 22.20 | O |
| ATOM | 132 | N | TYR | | 23 | 22.563 | 34.684 | −27.415 | 1.00 | 23.69 | N |
| ATOM | 133 | CA | TYR | | 23 | 21.856 | 34.991 | −28.649 | 1.00 | 22.30 | C |
| ATOM | 134 | CB | TYR | | 23 | 20.838 | 33.901 | −29.014 | 1.00 | 22.14 | C |
| ATOM | 135 | CG | TYR | | 23 | 19.604 | 33.801 | −28.143 | 1.00 | 23.90 | C |
| ATOM | 136 | CD1 | TYR | | 23 | 18.394 | 34.384 | −28.536 | 1.00 | 24.48 | C |
| ATOM | 137 | CE1 | TYR | | 23 | 17.236 | 34.223 | −27.777 | 1.00 | 23.42 | C |
| ATOM | 138 | CD2 | TYR | | 23 | 19.624 | 33.062 | −26.960 | 1.00 | 25.02 | C |
| ATOM | 139 | CE2 | TYR | | 23 | 18.478 | 32.896 | −26.196 | 1.00 | 27.09 | C |
| ATOM | 140 | CZ | TYR | | 23 | 17.286 | 33.475 | −26.610 | 1.00 | 26.36 | C |
| ATOM | 141 | OH | TYR | | 23 | 16.151 | 33.282 | −25.852 | 1.00 | 26.92 | O |
| ATOM | 142 | C | TYR | | 23 | 21.179 | 36.322 | −28.409 | 1.00 | 22.90 | C |
| ATOM | 143 | O | TYR | | 23 | 21.224 | 36.850 | −27.298 | 1.00 | 22.70 | O |
| ATOM | 144 | N | TYR | | 24 | 20.561 | 36.873 | −29.441 | 1.00 | 23.42 | N |
| ATOM | 145 | CA | TYR | | 24 | 19.916 | 38.161 | −29.289 | 1.00 | 23.78 | C |
| ATOM | 146 | CB | TYR | | 24 | 20.641 | 39.189 | −30.156 | 1.00 | 26.76 | C |
| ATOM | 147 | CG | TYR | | 24 | 22.094 | 39.296 | −29.751 | 1.00 | 31.08 | C |
| ATOM | 148 | CD1 | TYR | | 24 | 23.035 | 38.359 | −30.191 | 1.00 | 33.27 | C |
| ATOM | 149 | CE1 | TYR | | 24 | 24.353 | 38.387 | −29.729 | 1.00 | 34.88 | C |
| ATOM | 150 | CD2 | TYR | | 24 | 22.512 | 40.269 | −28.843 | 1.00 | 33.85 | C |
| ATOM | 151 | CE2 | TYR | | 24 | 23.825 | 40.308 | −28.375 | 1.00 | 34.75 | C |
| ATOM | 152 | CZ | TYR | | 24 | 24.740 | 39.366 | −28.820 | 1.00 | 36.76 | C |
| ATOM | 153 | OH | TYR | | 24 | 26.037 | 39.402 | −28.354 | 1.00 | 38.29 | O |
| ATOM | 154 | C | TYR | | 24 | 18.431 | 38.094 | −29.588 | 1.00 | 23.08 | C |
| ATOM | 155 | O | TYR | | 24 | 17.989 | 37.360 | −30.466 | 1.00 | 21.07 | O |
| ATOM | 156 | N | PHE | | 25 | 17.668 | 38.857 | −28.821 | 1.00 | 23.04 | N |
| ATOM | 157 | CA | PHE | | 25 | 16.224 | 38.887 | −28.942 | 1.00 | 23.59 | C |
| ATOM | 158 | CB | PHE | | 25 | 15.625 | 38.214 | −27.706 | 1.00 | 23.65 | C |
| ATOM | 159 | CG | PHE | | 25 | 14.153 | 38.413 | −27.550 | 1.00 | 24.29 | C |
| ATOM | 160 | CD1 | PHE | | 25 | 13.252 | 37.731 | −28.360 | 1.00 | 24.38 | C |
| ATOM | 161 | CD2 | PHE | | 25 | 13.663 | 39.279 | −26.575 | 1.00 | 24.35 | C |
| ATOM | 162 | CE1 | PHE | | 25 | 11.874 | 37.910 | −28.200 | 1.00 | 23.65 | C |
| ATOM | 163 | CE2 | PHE | | 25 | 12.296 | 39.464 | −26.409 | 1.00 | 24.27 | C |
| ATOM | 164 | CZ | PHE | | 25 | 11.400 | 38.776 | −27.225 | 1.00 | 24.79 | C |
| ATOM | 165 | C | PHE | | 25 | 15.756 | 40.333 | −29.055 | 1.00 | 24.00 | C |
| ATOM | 166 | O | PHE | | 25 | 16.287 | 41.219 | −28.386 | 1.00 | 24.15 | O |
| ATOM | 167 | N | ASN | | 26 | 14.773 | 40.564 | −29.921 | 1.00 | 23.32 | N |
| ATOM | 168 | CA | ASN | | 26 | 14.215 | 41.900 | −30.122 | 1.00 | 23.05 | C |
| ATOM | 169 | CB | ASN | | 26 | 14.169 | 42.231 | −31.621 | 1.00 | 22.84 | C |
| ATOM | 170 | CG | ASN | | 26 | 13.816 | 43.686 | −31.889 | 1.00 | 20.90 | C |
| ATOM | 171 | OD1 | ASN | | 26 | 12.734 | 44.146 | −31.542 | 1.00 | 20.54 | O |
| ATOM | 172 | ND2 | ASN | | 26 | 14.734 | 44.412 | −32.511 | 1.00 | 21.46 | N |
| ATOM | 173 | C | ASN | | 26 | 12.810 | 41.891 | −29.536 | 1.00 | 20.66 | C |
| ATOM | 174 | O | ASN | | 26 | 11.950 | 41.139 | −29.986 | 1.00 | 20.34 | O |
| ATOM | 175 | N | HIS | | 27 | 12.572 | 42.725 | −28.528 | 1.00 | 21.02 | N |
| ATOM | 176 | CA | HIS | | 27 | 11.266 | 42.747 | −27.880 | 1.00 | 20.66 | C |
| ATOM | 177 | CB | HIS | | 27 | 11.398 | 43.280 | −26.442 | 1.00 | 20.66 | C |
| ATOM | 178 | CG | HIS | | 27 | 11.962 | 44.665 | −26.348 | 1.00 | 23.34 | C |
| ATOM | 179 | CD2 | HIS | | 27 | 13.198 | 45.103 | −26.012 | 1.00 | 23.87 | C |
| ATOM | 180 | ND1 | HIS | | 27 | 11.211 | 45.795 | −26.597 | 1.00 | 25.84 | N |
| ATOM | 181 | CE1 | HIS | | 27 | 11.959 | 46.868 | −26.416 | 1.00 | 25.11 | C |
| ATOM | 182 | NE2 | HIS | | 27 | 13.170 | 46.476 | −26.062 | 1.00 | 27.78 | N |
| ATOM | 183 | C | HIS | | 27 | 10.170 | 43.498 | −28.637 | 1.00 | 20.96 | C |
| ATOM | 184 | O | HIS | | 27 | 9.031 | 43.556 | −28.187 | 1.00 | 21.38 | O |
| ATOM | 185 | N | ILE | | 28 | 10.507 | 44.063 | −29.786 | 1.00 | 22.76 | N |
| ATOM | 186 | CA | ILE | | 28 | 9.506 | 44.769 | −30.579 | 1.00 | 23.22 | C |
| ATOM | 187 | CB | ILE | | 28 | 10.077 | 46.083 | −31.149 | 1.00 | 23.38 | C |
| ATOM | 188 | CG2 | ILE | | 28 | 9.017 | 46.788 | −31.990 | 1.00 | 22.95 | C |
| ATOM | 189 | CG1 | ILE | | 28 | 10.515 | 46.990 | −29.993 | 1.00 | 23.21 | C |
| ATOM | 190 | CD1 | ILE | | 28 | 11.319 | 48.213 | −30.425 | 1.00 | 24.17 | C |
| ATOM | 191 | C | ILE | | 28 | 9.011 | 43.872 | −31.720 | 1.00 | 22.19 | C |
| ATOM | 192 | O | ILE | | 28 | 7.810 | 43.823 | −32.001 | 1.00 | 22.10 | O |
| ATOM | 193 | N | THR | | 29 | 9.931 | 43.138 | −32.347 | 1.00 | 21.09 | N |
| ATOM | 194 | CA | THR | | 29 | 9.582 | 42.237 | −33.452 | 1.00 | 21.14 | C |
| ATOM | 195 | CB | THR | | 29 | 10.655 | 42.249 | −34.551 | 1.00 | 21.56 | C |
| ATOM | 196 | OG1 | THR | | 29 | 11.887 | 41.750 | −34.008 | 1.00 | 18.52 | O |
| ATOM | 197 | CG2 | THR | | 29 | 10.877 | 43.657 | −35.075 | 1.00 | 21.14 | C |
| ATOM | 198 | C | THR | | 29 | 9.454 | 40.785 | −33.002 | 1.00 | 21.23 | C |
| ATOM | 199 | O | THR | | 29 | 8.899 | 39.949 | −33.725 | 1.00 | 19.43 | O |
| ATOM | 200 | N | ASN | | 30 | 9.977 | 40.499 | −31.810 | 1.00 | 21.08 | N |
| ATOM | 201 | CA | ASN | | 30 | 9.985 | 39.155 | −31.243 | 1.00 | 20.34 | C |
| ATOM | 202 | CB | ASN | | 30 | 8.563 | 38.590 | −31.168 | 1.00 | 20.23 | C |
| ATOM | 203 | CG | ASN | | 30 | 7.787 | 39.129 | −29.968 | 1.00 | 20.49 | C |
| ATOM | 204 | OD1 | ASN | | 30 | 6.559 | 39.252 | −29.999 | 1.00 | 20.76 | O |
| ATOM | 205 | ND2 | ASN | | 30 | 8.508 | 39.444 | −28.905 | 1.00 | 19.52 | N |
| ATOM | 206 | C | ASN | | 30 | 10.913 | 38.229 | −32.037 | 1.00 | 20.74 | C |
| ATOM | 207 | O | ASN | | 30 | 10.803 | 37.001 | −31.975 | 1.00 | 20.80 | O |
| ATOM | 208 | N | ALA | | 31 | 11.836 | 38.824 | −32.785 | 1.00 | 19.68 | N |
| ATOM | 209 | CA | ALA | | 31 | 12.798 | 38.032 | −33.548 | 1.00 | 20.16 | C |
| ATOM | 210 | CB | ALA | | 31 | 13.345 | 38.841 | −34.720 | 1.00 | 19.74 | C |
| ATOM | 211 | C | ALA | | 31 | 13.947 | 37.605 | −32.637 | 1.00 | 20.68 | C |
| ATOM | 212 | O | ALA | | 31 | 14.253 | 38.270 | −31.646 | 1.00 | 18.54 | O |
| ATOM | 213 | N | SER | | 32 | 14.566 | 36.477 | −32.969 | 1.00 | 19.88 | N |
| ATOM | 214 | CA | SER | | 32 | 15.709 | 35.983 | −32.220 | 1.00 | 21.20 | C |
| ATOM | 215 | CB | SER | | 32 | 15.278 | 34.911 | −31.207 | 1.00 | 19.65 | C |
| ATOM | 216 | OG | SER | | 32 | 14.486 | 33.901 | −31.803 | 1.00 | 25.48 | O |
| ATOM | 217 | C | SER | | 32 | 16.716 | 35.433 | −33.236 | 1.00 | 22.14 | C |
| ATOM | 218 | O | SER | | 32 | 16.332 | 34.801 | −34.219 | 1.00 | 21.52 | O |
| ATOM | 219 | N | GLN | | 33 | 17.999 | 35.702 | −33.004 | 1.00 | 22.52 | N |
| ATOM | 220 | CA | GLN | | 33 | 19.072 | 35.271 | −33.902 | 1.00 | 23.90 | C |
| ATOM | 221 | CB | GLN | | 33 | 19.341 | 36.356 | −34.951 | 1.00 | 25.32 | C |
| ATOM | 222 | CG | GLN | | 33 | 19.529 | 37.743 | −34.344 | 1.00 | 28.58 | C |
| ATOM | 223 | CD | GLN | | 33 | 19.893 | 38.811 | −35.367 | 1.00 | 32.66 | C |
| ATOM | 224 | OE1 | GLN | | 33 | 21.068 | 39.118 | −35.572 | 1.00 | 36.83 | O |
| ATOM | 225 | NE2 | GLN | | 33 | 18.886 | 39.375 | −36.016 | 1.00 | 29.57 | N |
| ATOM | 226 | C | GLN | | 33 | 20.347 | 35.036 | −33.094 | 1.00 | 24.47 | C |
| ATOM | 227 | O | GLN | | 33 | 20.493 | 35.574 | −31.999 | 1.00 | 23.29 | O |
| ATOM | 228 | N | TRP | | 34 | 21.276 | 34.254 | −33.644 | 1.00 | 25.01 | N |
| ATOM | 229 | CA | TRP | | 34 | 22.531 | 33.973 | −32.948 | 1.00 | 25.47 | C |
| ATOM | 230 | CB | TRP | | 34 | 23.213 | 32.726 | −33.537 | 1.00 | 23.87 | C |
| ATOM | 231 | CG | TRP | | 34 | 22.521 | 31.438 | −33.188 | 1.00 | 21.81 | C |
| ATOM | 232 | CD2 | TRP | | 34 | 22.341 | 30.889 | −31.877 | 1.00 | 22.45 | C |
| ATOM | 233 | CE2 | TRP | | 34 | 21.590 | 29.702 | −32.021 | 1.00 | 22.25 | C |
| ATOM | 234 | CE3 | TRP | | 34 | 22.742 | 31.286 | −30.594 | 1.00 | 22.29 | C |
| ATOM | 235 | CD1 | TRP | | 34 | 21.896 | 30.582 | −34.049 | 1.00 | 21.58 | C |
| ATOM | 236 | NE1 | TRP | | 34 | 21.333 | 29.540 | −33.357 | 1.00 | 21.07 | N |
| ATOM | 237 | CZ2 | TRP | | 34 | 21.228 | 28.906 | −30.929 | 1.00 | 23.45 | C |
| ATOM | 238 | CZ3 | TRP | | 34 | 22.386 | 30.493 | −29.508 | 1.00 | 23.48 | C |
| ATOM | 239 | CH2 | TRP | | 34 | 21.635 | 29.317 | −29.683 | 1.00 | 23.38 | C |
| ATOM | 240 | C | TRP | | 34 | 23.518 | 35.140 | −32.965 | 1.00 | 27.36 | C |
| ATOM | 241 | O | TRP | | 34 | 24.168 | 35.417 | −31.954 | 1.00 | 26.42 | O |
| ATOM | 242 | N | GLU | | 35 | 23.624 | 35.827 | −34.103 | 1.00 | 28.79 | N |
| ATOM | 243 | CA | GLU | | 35 | 24.558 | 36.938 | −34.228 | 1.00 | 32.29 | C |
| ATOM | 244 | CB | GLU | | 35 | 24.885 | 37.205 | −35.694 | 1.00 | 32.69 | C |
| ATOM | 245 | CG | GLU | | 35 | 25.387 | 36.002 | −36.457 | 1.00 | 33.59 | C |
| ATOM | 246 | CD | GLU | | 35 | 26.098 | 36.391 | −37.735 | 1.00 | 34.42 | C |
| ATOM | 247 | OE1 | GLU | | 35 | 26.165 | 35.550 | −38.653 | 1.00 | 34.44 | O |
| ATOM | 248 | OE2 | GLU | | 35 | 26.607 | 37.534 | −37.815 | 1.00 | 33.46 | O |
| ATOM | 249 | C | GLU | | 35 | 24.084 | 38.238 | −33.604 | 1.00 | 36.01 | C |
| ATOM | 250 | O | GLU | | 35 | 22.887 | 38.518 | −33.549 | 1.00 | 34.67 | O |
| ATOM | 251 | N | ARG | | 36 | 25.043 | 39.033 | −33.142 | 1.00 | 39.87 | N |
| ATOM | 252 | CA | ARG | | 36 | 24.739 | 40.319 | −32.539 | 1.00 | 45.62 | C |
| ATOM | 253 | CB | ARG | | 36 | 25.973 | 40.886 | −31.837 | 1.00 | 47.70 | C |
| ATOM | 254 | CG | ARG | | 36 | 25.677 | 42.099 | −30.977 | 1.00 | 52.10 | C |
| ATOM | 255 | CD | ARG | | 36 | 26.885 | 42.506 | −30.154 | 1.00 | 55.23 | C |
| ATOM | 256 | NE | ARG | | 36 | 26.518 | 43.444 | −29.099 | 1.00 | 57.97 | N |
| ATOM | 257 | CZ | ARG | | 36 | 27.353 | 43.885 | −28.166 | 1.00 | 59.67 | C |
| ATOM | 258 | NH1 | ARG | | 36 | 28.615 | 43.475 | −28.155 | 1.00 | 60.43 | N |
| ATOM | 259 | NH2 | ARG | | 36 | 26.924 | 44.730 | −27.238 | 1.00 | 60.07 | N |
| ATOM | 260 | C | ARG | | 36 | 24.325 | 41.219 | −33.693 | 1.00 | 47.90 | C |
| ATOM | 261 | O | ARG | | 36 | 25.099 | 41.438 | −34.625 | 1.00 | 48.07 | O |
| ATOM | 262 | N | PRO | | 37 | 23.094 | 41.746 | −33.647 | 1.00 | 49.97 | N |
| ATOM | 263 | CD | PRO | | 37 | 22.208 | 41.730 | −32.471 | 1.00 | 50.02 | C |
| ATOM | 264 | CA | PRO | | 37 | 22.547 | 42.624 | −34.685 | 1.00 | 52.82 | C |
| ATOM | 265 | CB | PRO | | 37 | 21.156 | 42.947 | −34.153 | 1.00 | 51.49 | C |
| ATOM | 266 | CG | PRO | | 37 | 21.381 | 42.976 | −32.682 | 1.00 | 50.64 | C |
| ATOM | 267 | C | PRO | | 37 | 23.367 | 43.882 | −34.952 | 1.00 | 55.66 | C |
| ATOM | 268 | O | PRO | | 37 | 23.945 | 44.467 | −34.034 | 1.00 | 55.81 | O |
| ATOM | 269 | N | SER | | 38 | 23.403 | 44.284 | −36.220 | 1.00 | 58.07 | N |
| ATOM | 270 | CA | SER | | 38 | 24.126 | 45.474 | −36.658 | 1.00 | 60.06 | C |
| ATOM | 271 | CB | SER | | 38 | 23.196 | 46.689 | −36.607 | 1.00 | 59.17 | C |
| ATOM | 272 | OG | SER | | 38 | 22.011 | 46.444 | −37.347 | 1.00 | 57.19 | O |
| ATOM | 273 | C | SER | | 38 | 25.377 | 45.750 | −35.831 | 1.00 | 61.25 | C |
| ATOM | 274 | O | SER | | 38 | 25.633 | 46.888 | −35.437 | 1.00 | 62.84 | O |
| ATOM | 275 | N | GLU | | 51 | 14.438 | 56.608 | −39.353 | 1.00 | 57.69 | N |
| ATOM | 276 | CA | GLU | | 51 | 13.791 | 55.417 | −39.887 | 1.00 | 57.34 | C |
| ATOM | 277 | CB | GLU | | 51 | 12.787 | 54.856 | −38.872 | 1.00 | 57.35 | C |
| ATOM | 278 | CG | GLU | | 51 | 11.476 | 55.629 | −38.793 | 1.00 | 56.42 | C |
| ATOM | 279 | CD | GLU | | 51 | 10.543 | 55.101 | −37.718 | 1.00 | 56.15 | C |
| ATOM | 280 | OE1 | GLU | | 51 | 9.364 | 55.509 | −37.704 | 1.00 | 55.24 | O |
| ATOM | 281 | OE2 | GLU | | 51 | 10.987 | 54.285 | −36.884 | 1.00 | 56.66 | O |
| ATOM | 282 | C | GLU | | 51 | 13.066 | 55.776 | −41.180 | 1.00 | 56.81 | C |
| ATOM | 283 | O | GLU | | 51 | 12.765 | 56.944 | −41.425 | 1.00 | 57.20 | O |
| ATOM | 284 | N | PRO | | 52 | 12.776 | 54.774 | −42.026 | 1.00 | 55.89 | N |
| ATOM | 285 | CD | PRO | | 52 | 13.163 | 53.358 | −41.904 | 1.00 | 55.54 | C |
| ATOM | 286 | CA | PRO | | 52 | 12.081 | 55.013 | −43.294 | 1.00 | 54.05 | C |
| ATOM | 287 | CB | PRO | | 52 | 11.985 | 53.617 | −43.903 | 1.00 | 54.81 | C |
| ATOM | 288 | CG | PRO | | 52 | 13.186 | 52.919 | −43.344 | 1.00 | 55.46 | C |
| ATOM | 289 | C | PRO | | 52 | 10.706 | 55.626 | −43.066 | 1.00 | 52.40 | C |
| ATOM | 290 | O | PRO | | 52 | 10.054 | 55.342 | −42.064 | 1.00 | 53.30 | O |
| ATOM | 291 | N | ALA | | 53 | 10.269 | 56.467 | −43.995 | 1.00 | 50.01 | N |
| ATOM | 292 | CA | ALA | | 53 | 8.962 | 57.096 | −43.882 | 1.00 | 47.50 | C |
| ATOM | 293 | CB | ALA | | 53 | 8.839 | 58.233 | −44.888 | 1.00 | 47.78 | C |
| ATOM | 294 | C | ALA | | 53 | 7.902 | 56.037 | −44.151 | 1.00 | 45.45 | C |
| ATOM | 295 | O | ALA | | 53 | 6.777 | 56.115 | −43.654 | 1.00 | 44.38 | O |
| ATOM | 296 | N | ARG | | 54 | 8.277 | 55.044 | −44.949 | 1.00 | 43.10 | N |
| ATOM | 297 | CA | ARG | | 54 | 7.380 | 53.954 | −45.301 | 1.00 | 39.82 | C |
| ATOM | 298 | CB | ARG | | 54 | 6.669 | 54.259 | −46.623 | 1.00 | 42.74 | C |
| ATOM | 299 | CG | ARG | | 54 | 5.840 | 55.532 | −46.615 | 1.00 | 46.81 | C |
| ATOM | 300 | CD | ARG | | 54 | 5.537 | 56.006 | −48.032 | 1.00 | 49.87 | C |
| ATOM | 301 | NE | ARG | | 54 | 4.699 | 55.078 | −48.790 | 1.00 | 51.01 | N |
| ATOM | 302 | CZ | ARG | | 54 | 3.428 | 54.811 | −48.507 | 1.00 | 52.78 | C |
| ATOM | 303 | NH1 | ARG | | 54 | 2.834 | 55.398 | −47.476 | 1.00 | 53.46 | N |
| ATOM | 304 | NH2 | ARG | | 54 | 2.743 | 53.965 | −49.266 | 1.00 | 53.28 | N |
| ATOM | 305 | C | ARG | | 54 | 8.171 | 52.661 | −45.450 | 1.00 | 35.96 | C |
| ATOM | 306 | O | ARG | | 54 | 9.373 | 52.676 | −45.712 | 1.00 | 35.46 | O |
| ATOM | 307 | N | VAL | | 55 | 7.489 | 51.540 | −45.266 | 1.00 | 32.57 | N |
| ATOM | 308 | CA | VAL | | 55 | 8.110 | 50.235 | −45.429 | 1.00 | 28.54 | C |
| ATOM | 309 | CB | VAL | | 55 | 8.445 | 49.557 | −44.071 | 1.00 | 27.23 | C |
| ATOM | 310 | CG1 | VAL | | 55 | 9.463 | 50.390 | −43.307 | 1.00 | 24.86 | C |
| ATOM | 311 | CG2 | VAL | | 55 | 7.170 | 49.373 | −43.251 | 1.00 | 24.27 | C |
| ATOM | 312 | C | VAL | | 55 | 7.094 | 49.373 | −46.149 | 1.00 | 27.45 | C |
| ATOM | 313 | O | VAL | | 55 | 5.902 | 49.681 | −46.163 | 1.00 | 26.43 | O |
| ATOM | 314 | N | ARG | | 56 | 7.571 | 48.301 | −46.764 | 1.00 | 25.29 | N |
| ATOM | 315 | CA | ARG | | 56 | 6.687 | 47.379 | −47.449 | 1.00 | 23.95 | C |
| ATOM | 316 | CB | ARG | | 56 | 7.073 | 47.251 | −48.924 | 1.00 | 23.09 | C |
| ATOM | 317 | CG | ARG | | 56 | 6.146 | 46.336 | −49.702 | 1.00 | 24.25 | C |
| ATOM | 318 | CD | ARG | | 56 | 6.558 | 46.212 | −51.166 | 1.00 | 25.63 | C |
| ATOM | 319 | NE | ARG | | 56 | 5.648 | 45.336 | −51.900 | 1.00 | 26.11 | N |
| ATOM | 320 | CZ | ARG | | 56 | 5.716 | 45.108 | −53.210 | 1.00 | 27.42 | C |
| ATOM | 321 | NH1 | ARG | | 56 | 6.656 | 45.691 | −53.944 | 1.00 | 25.82 | N |
| ATOM | 322 | NH2 | ARG | | 56 | 4.841 | 44.297 | −53.789 | 1.00 | 26.73 | N |
| ATOM | 323 | C | ARG | | 56 | 6.852 | 46.041 | −46.742 | 1.00 | 22.60 | C |
| ATOM | 324 | O | ARG | | 56 | 7.973 | 45.599 | −46.497 | 1.00 | 22.92 | O |
| ATOM | 325 | N | CYS | | 57 | 5.740 | 45.405 | −46.401 | 1.00 | 22.50 | N |
| ATOM | 326 | CA | CYS | | 57 | 5.797 | 44.124 | −45.715 | 1.00 | 21.96 | C |
| ATOM | 327 | CB | CYS | | 57 | 5.526 | 44.299 | −44.216 | 1.00 | 21.34 | C |
| ATOM | 328 | SG | CYS | | 57 | 6.684 | 45.319 | −43.331 | 1.00 | 20.11 | S |
| ATOM | 329 | C | CYS | | 57 | 4.773 | 43.148 | −46.240 | 1.00 | 22.55 | C |
| ATOM | 330 | O | CYS | | 57 | 3.781 | 43.534 | −46.871 | 1.00 | 22.64 | O |
| ATOM | 331 | N | SER | | 58 | 5.037 | 41.875 | −45.972 | 1.00 | 20.32 | N |
| ATOM | 332 | CA | SER | | 58 | 4.123 | 40.796 | −46.298 | 1.00 | 20.21 | C |
| ATOM | 333 | CB | SER | | 58 | 4.763 | 39.753 | −47.217 | 1.00 | 20.04 | C |
| ATOM | 334 | OG | SER | | 58 | 5.040 | 40.301 | −48.486 | 1.00 | 19.12 | O |
| ATOM | 335 | C | SER | | 58 | 3.893 | 40.179 | −44.932 | 1.00 | 20.61 | C |
| ATOM | 336 | O | SER | | 58 | 4.712 | 40.362 | −44.019 | 1.00 | 20.53 | O |
| ATOM | 337 | N | HIS | | 59 | 2.788 | 39.462 | −44.770 | 1.00 | 20.02 | N |
| ATOM | 338 | CA | HIS | | 59 | 2.544 | 38.821 | −43.499 | 1.00 | 19.93 | C |
| ATOM | 339 | CB | HIS | | 59 | 1.860 | 39.802 | −42.515 | 1.00 | 21.80 | C |
| ATOM | 340 | CG | HIS | | 59 | 0.362 | 39.853 | −42.617 | 1.00 | 24.62 | C |
| ATOM | 341 | CD2 | HIS | | 59 | −0.592 | 39.810 | −41.657 | 1.00 | 22.95 | C |
| ATOM | 342 | ND1 | HIS | | 59 | −0.307 | 40.000 | −43.813 | 1.00 | 24.31 | N |
| ATOM | 343 | CE1 | HIS | | 59 | −1.609 | 40.041 | −43.586 | 1.00 | 21.97 | C |
| ATOM | 344 | NE2 | HIS | | 59 | −1.809 | 39.930 | −42.286 | 1.00 | 26.77 | N |
| ATOM | 345 | C | HIS | | 59 | 1.735 | 37.555 | −43.672 | 1.00 | 20.16 | C |
| ATOM | 346 | O | HIS | | 59 | 1.118 | 37.315 | −44.720 | 1.00 | 18.78 | O |
| ATOM | 347 | N | LEU | | 60 | 1.789 | 36.720 | −42.647 | 1.00 | 18.36 | N |
| ATOM | 348 | CA | LEU | | 60 | 1.043 | 35.482 | −42.616 | 1.00 | 19.49 | C |
| ATOM | 349 | CB | LEU | | 60 | 1.976 | 34.278 | −42.576 | 1.00 | 18.97 | C |
| ATOM | 350 | CG | LEU | | 60 | 1.345 | 32.883 | −42.703 | 1.00 | 18.12 | C |
| ATOM | 351 | CD1 | LEU | | 60 | 2.445 | 31.884 | −43.007 | 1.00 | 18.41 | C |
| ATOM | 352 | CD2 | LEU | | 60 | 0.605 | 32.495 | −41.431 | 1.00 | 17.46 | C |
| ATOM | 353 | C | LEU | | 60 | 0.293 | 35.625 | −41.305 | 1.00 | 20.43 | C |
| ATOM | 354 | O | LEU | | 60 | 0.894 | 35.803 | −40.240 | 1.00 | 19.13 | O |
| ATOM | 355 | N | LEU | | 61 | −1.026 | 35.592 | −41.391 | 1.00 | 20.07 | N |
| ATOM | 356 | CA | LEU | | 61 | −1.850 | 35.751 | −40.210 | 1.00 | 21.15 | C |
| ATOM | 357 | CB | LEU | | 61 | −2.889 | 36.849 | −40.457 | 1.00 | 22.48 | C |
| ATOM | 358 | CG | LEU | | 61 | −4.014 | 37.017 | −39.429 | 1.00 | 22.65 | C |
| ATOM | 359 | CD1 | LEU | | 61 | −3.441 | 37.533 | −38.115 | 1.00 | 24.95 | C |
| ATOM | 360 | CD2 | LEU | | 61 | −5.060 | 37.990 | −39.972 | 1.00 | 24.74 | C |
| ATOM | 361 | C | LEU | | 61 | −2.556 | 34.460 | −39.843 | 1.00 | 20.90 | C |
| ATOM | 362 | O | LEU | | 61 | −3.067 | 33.749 | −40.705 | 1.00 | 18.95 | O |
| ATOM | 363 | N | VAL | | 62 | −2.557 | 34.147 | −38.554 | 1.00 | 20.75 | N |
| ATOM | 364 | CA | VAL | | 62 | −3.261 | 32.978 | −38.075 | 1.00 | 20.88 | C |
| ATOM | 365 | CB | VAL | | 62 | −2.324 | 31.949 | −37.415 | 1.00 | 21.62 | C |
| ATOM | 366 | CG1 | VAL | | 62 | −3.155 | 30.825 | −36.786 | 1.00 | 21.94 | C |
| ATOM | 367 | CG2 | VAL | | 62 | −1.382 | 31.361 | −38.461 | 1.00 | 22.40 | C |
| ATOM | 368 | C | VAL | | 62 | −4.234 | 33.540 | −37.047 | 1.00 | 21.88 | C |
| ATOM | 369 | O | VAL | | 62 | −3.827 | 34.113 | −36.037 | 1.00 | 19.51 | O |
| ATOM | 370 | N | LYS | | 63 | −5.523 | 33.411 | −37.334 | 1.00 | 22.77 | N |
| ATOM | 371 | CA | LYS | | 63 | −6.543 | 33.923 | −36.434 | 1.00 | 25.20 | C |
| ATOM | 372 | CB | LYS | | 63 | −7.782 | 34.376 | −37.218 | 1.00 | 26.39 | C |
| ATOM | 373 | CG | LYS | | 63 | −7.604 | 35.675 | −37.987 | 1.00 | 28.45 | C |
| ATOM | 374 | CD | LYS | | 63 | −8.925 | 36.124 | −38.611 | 1.00 | 29.09 | C |
| ATOM | 375 | CE | LYS | | 63 | −8.822 | 37.513 | −39.201 | 1.00 | 27.74 | C |
| ATOM | 376 | NZ | LYS | | 63 | −10.093 | 37.921 | −39.861 | 1.00 | 28.12 | N |
| ATOM | 377 | C | LYS | | 63 | −6.950 | 32.873 | −35.415 | 1.00 | 25.23 | C |
| ATOM | 378 | O | LYS | | 63 | −6.698 | 31.681 | −35.591 | 1.00 | 24.50 | O |
| ATOM | 379 | N | HIS | | 64 | −7.573 | 33.335 | −34.339 | 1.00 | 26.37 | N |
| ATOM | 380 | CA | HIS | | 64 | −8.046 | 32.449 | −33.288 | 1.00 | 26.91 | C |
| ATOM | 381 | CB | HIS | | 64 | −7.077 | 32.470 | −32.098 | 1.00 | 24.05 | C |
| ATOM | 382 | CG | HIS | | 64 | −6.700 | 33.845 | −31.646 | 1.00 | 25.82 | C |
| ATOM | 383 | CD2 | HIS | | 64 | −5.538 | 34.531 | −31.753 | 1.00 | 26.61 | C |
| ATOM | 384 | ND1 | HIS | | 64 | −7.584 | 34.688 | −31.005 | 1.00 | 25.59 | N |
| ATOM | 385 | CE1 | HIS | | 64 | −6.982 | 35.832 | −30.738 | 1.00 | 28.49 | C |
| ATOM | 386 | NE2 | HIS | | 64 | −5.739 | 35.763 | −31.182 | 1.00 | 27.26 | N |
| ATOM | 387 | C | HIS | | 64 | −9.448 | 32.903 | −32.882 | 1.00 | 27.07 | C |
| ATOM | 388 | O | HIS | | 64 | −9.980 | 33.858 | −33.446 | 1.00 | 26.03 | O |
| ATOM | 389 | N | SER | | 65 | −10.044 | 32.223 | −31.910 | 1.00 | 30.47 | N |
| ATOM | 390 | CA | SER | | 65 | −11.395 | 32.555 | −31.472 | 1.00 | 30.56 | C |
| ATOM | 391 | CB | SER | | 65 | −11.891 | 31.508 | −30.473 | 1.00 | 33.30 | C |
| ATOM | 392 | OG | SER | | 65 | −11.143 | 31.559 | −29.272 | 1.00 | 34.85 | O |
| ATOM | 393 | C | SER | | 65 | −11.551 | 33.952 | −30.868 | 1.00 | 31.48 | C |
| ATOM | 394 | O | SER | | 65 | −12.665 | 34.473 | −30.796 | 1.00 | 30.78 | O |
| ATOM | 395 | N | GLN | | 66 | −10.455 | 34.564 | −30.430 | 1.00 | 29.16 | N |
| ATOM | 396 | CA | GLN | | 66 | −10.554 | 35.901 | −29.846 | 1.00 | 29.95 | C |
| ATOM | 397 | CB | GLN | | 66 | −9.703 | 36.005 | −28.567 | 1.00 | 30.78 | C |
| ATOM | 398 | CG | GLN | | 66 | −10.244 | 35.180 | −27.394 | 1.00 | 31.19 | C |
| ATOM | 399 | CD | GLN | | 66 | −9.508 | 35.439 | −26.078 | 1.00 | 34.58 | C |
| ATOM | 400 | OE1 | GLN | | 66 | −9.476 | 36.568 | −25.577 | 1.00 | 32.21 | O |
| ATOM | 401 | NE2 | GLN | | 66 | −8.921 | 34.388 | −25.512 | 1.00 | 34.03 | N |
| ATOM | 402 | C | GLN | | 66 | −10.192 | 37.025 | −30.814 | 1.00 | 29.66 | C |
| ATOM | 403 | O | GLN | | 66 | −10.088 | 38.187 | −30.414 | 1.00 | 32.44 | O |
| ATOM | 404 | N | SER | | 67 | −10.002 | 36.688 | −32.087 | 1.00 | 30.14 | N |
| ATOM | 405 | CA | SER | | 67 | −9.676 | 37.694 | −33.096 | 1.00 | 31.52 | C |
| ATOM | 406 | CB | SER | | 67 | −9.325 | 37.024 | −34.432 | 1.00 | 29.84 | C |
| ATOM | 407 | OG | SER | | 67 | −8.112 | 36.294 | −34.338 | 1.00 | 26.70 | O |
| ATOM | 408 | C | SER | | 67 | −10.902 | 38.585 | −33.271 | 1.00 | 33.64 | C |
| ATOM | 409 | O | SER | | 67 | −12.031 | 38.094 | −33.203 | 1.00 | 33.54 | O |
| ATOM | 410 | N | ARG | | 68 | −10.687 | 39.880 | −33.499 | 1.00 | 36.87 | N |
| ATOM | 411 | CA | ARG | | 68 | −11.808 | 40.805 | −33.658 | 1.00 | 41.36 | C |
| ATOM | 412 | CB | ARG | | 68 | −11.325 | 42.189 | −34.103 | 1.00 | 45.22 | C |
| ATOM | 413 | CG | ARG | | 68 | −10.498 | 42.197 | −35.362 | 1.00 | 52.20 | C |
| ATOM | 414 | CD | ARG | | 68 | −10.198 | 43.620 | −35.816 | 1.00 | 57.28 | C |
| ATOM | 415 | NE | ARG | | 68 | −9.482 | 44.391 | −34.805 | 1.00 | 61.00 | N |
| ATOM | 416 | CZ | ARG | | 68 | −8.980 | 45.604 | −35.011 | 1.00 | 63.18 | C |
| ATOM | 417 | NH1 | ARG | | 68 | −9.112 | 46.186 | −36.197 | 1.00 | 63.39 | N |
| ATOM | 418 | NH2 | ARG | | 68 | −8.347 | 46.238 | −34.032 | 1.00 | 64.64 | N |
| ATOM | 419 | C | ARG | | 68 | −12.832 | 40.252 | −34.641 | 1.00 | 41.19 | C |
| ATOM | 420 | O | ARG | | 68 | −14.035 | 40.431 | −34.454 | 1.00 | 41.37 | O |
| ATOM | 421 | N | ARG | | 69 | −12.350 | 39.572 | −35.678 | 1.00 | 40.86 | N |
| ATOM | 422 | CA | ARG | | 69 | −13.217 | 38.955 | −36.679 | 1.00 | 39.53 | C |
| ATOM | 423 | CB | ARG | | 69 | −13.147 | 39.729 | −37.995 | 1.00 | 43.41 | C |
| ATOM | 424 | CG | ARG | | 69 | −14.046 | 40.953 | −38.043 | 1.00 | 47.58 | C |
| ATOM | 425 | CD | ARG | | 69 | −13.773 | 41.777 | −39.287 | 1.00 | 52.27 | C |
| ATOM | 426 | NE | ARG | | 69 | −13.590 | 40.936 | −40.466 | 1.00 | 56.08 | N |
| ATOM | 427 | CZ | ARG | | 69 | −13.277 | 41.396 | −41.673 | 1.00 | 58.81 | C |
| ATOM | 428 | NH1 | ARG | | 69 | −13.114 | 42.698 | −41.865 | 1.00 | 60.54 | N |
| ATOM | 429 | NH2 | ARG | | 69 | −13.119 | 40.556 | −42.688 | 1.00 | 59.95 | N |
| ATOM | 430 | C | ARG | | 69 | −12.772 | 37.508 | −36.893 | 1.00 | 37.96 | C |
| ATOM | 431 | O | ARG | | 69 | −11.934 | 37.227 | −37.751 | 1.00 | 36.44 | O |
| ATOM | 432 | N | PRO | | 70 | −13.330 | 36.570 | −36.106 | 1.00 | 36.89 | N |
| ATOM | 433 | CD | PRO | | 70 | −14.289 | 36.845 | −35.021 | 1.00 | 38.20 | C |
| ATOM | 434 | CA | PRO | | 70 | −13.019 | 35.137 | −36.166 | 1.00 | 35.97 | C |
| ATOM | 435 | CB | PRO | | 70 | −13.655 | 34.598 | −34.887 | 1.00 | 37.31 | C |
| ATOM | 436 | CG | PRO | | 70 | −14.851 | 35.471 | −34.729 | 1.00 | 37.53 | C |
| ATOM | 437 | C | PRO | | 70 | −13.515 | 34.415 | −37.414 | 1.00 | 34.97 | C |
| ATOM | 438 | O | PRO | | 70 | −14.282 | 33.447 | −37.332 | 1.00 | 34.38 | O |
| ATOM | 439 | N | SER | | 71 | −13.047 | 34.874 | −38.570 | 1.00 | 32.94 | N |
| ATOM | 440 | CA | SER | | 71 | −13.435 | 34.287 | −39.848 | 1.00 | 30.60 | C |
| ATOM | 441 | CB | SER | | 71 | −14.826 | 34.797 | −40.245 | 1.00 | 32.20 | C |
| ATOM | 442 | OG | SER | | 71 | −15.220 | 34.309 | −41.516 | 1.00 | 37.57 | O |
| ATOM | 443 | C | SER | | 71 | −12.411 | 34.703 | −40.898 | 1.00 | 28.59 | C |
| ATOM | 444 | O | SER | | 71 | −11.703 | 35.691 | −40.710 | 1.00 | 27.43 | O |
| ATOM | 445 | N | SER | | 72 | −12.328 | 33.941 | −41.985 | 1.00 | 27.81 | N |
| ATOM | 446 | CA | SER | | 72 | −11.406 | 34.240 | −43.086 | 1.00 | 28.26 | C |
| ATOM | 447 | CB | SER | | 72 | −9.952 | 33.918 | −42.703 | 1.00 | 25.68 | C |
| ATOM | 448 | OG | SER | | 72 | −9.714 | 32.518 | −42.730 | 1.00 | 21.94 | O |
| ATOM | 449 | C | SER | | 72 | −11.797 | 33.400 | −44.295 | 1.00 | 27.75 | C |
| ATOM | 450 | O | SER | | 72 | −12.712 | 32.586 | −44.215 | 1.00 | 27.95 | O |
| ATOM | 451 | N | TRP | | 73 | −11.087 | 33.584 | −45.405 | 1.00 | 28.49 | N |
| ATOM | 452 | CA | TRP | | 73 | −11.375 | 32.838 | −46.621 | 1.00 | 27.17 | C |
| ATOM | 453 | CB | TRP | | 73 | −10.521 | 33.351 | −47.792 | 1.00 | 27.62 | C |
| ATOM | 454 | CG | TRP | | 73 | −9.035 | 33.087 | −47.648 | 1.00 | 28.44 | C |
| ATOM | 455 | CD2 | TRP | | 73 | −8.326 | 31.921 | −48.093 | 1.00 | 27.51 | C |
| ATOM | 456 | CE2 | TRP | | 73 | −6.975 | 32.082 | −47.714 | 1.00 | 26.68 | C |
| ATOM | 457 | CE3 | TRP | | 73 | −8.703 | 30.755 | −48.773 | 1.00 | 27.78 | C |
| ATOM | 458 | CD1 | TRP | | 73 | −8.107 | 33.887 | −47.035 | 1.00 | 27.25 | C |
| ATOM | 459 | NE1 | TRP | | 73 | −6.868 | 33.288 | −47.071 | 1.00 | 26.15 | N |
| ATOM | 460 | CZ2 | TRP | | 73 | −5.997 | 31.118 | −47.994 | 1.00 | 26.40 | C |
| ATOM | 461 | CZ3 | TRP | | 73 | −7.726 | 29.794 | −49.050 | 1.00 | 27.70 | C |
| ATOM | 462 | CH2 | TRP | | 73 | −6.392 | 29.986 | −48.658 | 1.00 | 26.52 | C |
| ATOM | 463 | C | TRP | | 73 | −11.121 | 31.352 | −46.419 | 1.00 | 26.91 | C |
| ATOM | 464 | O | TRP | | 73 | −11.669 | 30.523 | −47.139 | 1.00 | 27.03 | O |
| ATOM | 465 | N | ARG | | 74 | −10.298 | 31.017 | −45.429 | 1.00 | 26.23 | N |
| ATOM | 466 | CA | ARG | | 74 | −9.968 | 29.624 | −45.146 | 1.00 | 25.74 | C |
| ATOM | 467 | CB | ARG | | 74 | −8.696 | 29.536 | −44.300 | 1.00 | 23.12 | C |
| ATOM | 468 | CG | ARG | | 74 | −7.503 | 30.297 | −44.871 | 1.00 | 22.48 | C |
| ATOM | 469 | CD | ARG | | 74 | −6.217 | 29.755 | −44.266 | 1.00 | 22.09 | C |
| ATOM | 470 | NE | ARG | | 74 | −6.071 | 28.352 | −44.633 | 1.00 | 22.25 | N |
| ATOM | 471 | CZ | ARG | | 74 | −5.729 | 27.386 | −43.792 | 1.00 | 23.61 | C |
| ATOM | 472 | NH1 | ARG | | 74 | −5.489 | 27.663 | −42.517 | 1.00 | 27.30 | N |
| ATOM | 473 | NH2 | ARG | | 74 | −5.641 | 26.138 | −44.230 | 1.00 | 25.35 | N |
| ATOM | 474 | C | ARG | | 74 | −11.094 | 28.882 | −44.434 | 1.00 | 26.69 | C |
| ATOM | 475 | O | ARG | | 74 | −11.276 | 27.685 | −44.640 | 1.00 | 25.48 | O |
| ATOM | 476 | N | GLN | | 75 | −11.825 | 29.583 | −43.573 | 1.00 | 28.87 | N |
| ATOM | 477 | CA | GLN | | 75 | −12.942 | 28.970 | −42.861 | 1.00 | 31.31 | C |
| ATOM | 478 | CB | GLN | | 75 | −12.450 | 28.048 | −41.740 | 1.00 | 33.76 | C |
| ATOM | 479 | CG | GLN | | 75 | −11.543 | 28.688 | −40.727 | 1.00 | 37.08 | C |
| ATOM | 480 | CD | GLN | | 75 | −11.212 | 27.745 | −39.581 | 1.00 | 39.40 | C |
| ATOM | 481 | OE1 | GLN | | 75 | −10.375 | 28.053 | −38.731 | 1.00 | 39.41 | O |
| ATOM | 482 | NE2 | GLN | | 75 | −11.875 | 26.593 | −39.549 | 1.00 | 38.99 | N |
| ATOM | 483 | C | GLN | | 75 | −13.885 | 30.030 | −42.307 | 1.00 | 29.89 | C |
| ATOM | 484 | O | GLN | | 75 | −13.457 | 31.011 | −41.703 | 1.00 | 28.98 | O |
| ATOM | 485 | N | GLU | | 76 | −15.180 | 29.815 | −42.524 | 1.00 | 30.41 | N |
| ATOM | 486 | CA | GLU | | 76 | −16.207 | 30.758 | −42.102 | 1.00 | 28.77 | C |
| ATOM | 487 | CB | GLU | | 76 | −17.589 | 30.246 | −42.537 | 1.00 | 28.89 | C |
| ATOM | 488 | CG | GLU | | 76 | −18.658 | 31.332 | −42.562 | 1.00 | 28.30 | C |
| ATOM | 489 | CD | GLU | | 76 | −19.925 | 30.905 | −43.299 | 1.00 | 28.54 | C |
| ATOM | 490 | OE1 | GLU | | 76 | −20.809 | 31.764 | −43.496 | 1.00 | 28.39 | O |
| ATOM | 491 | OE2 | GLU | | 76 | −20.034 | 29.718 | −43.681 | 1.00 | 25.51 | O |
| ATOM | 492 | C | GLU | | 76 | −16.211 | 31.078 | −40.610 | 1.00 | 29.59 | C |
| ATOM | 493 | O | GLU | | 76 | −16.461 | 32.215 | −40.215 | 1.00 | 28.90 | O |
| ATOM | 494 | N | LYS | | 77 | −15.932 | 30.082 | −39.783 | 1.00 | 31.59 | N |
| ATOM | 495 | CA | LYS | | 77 | −15.918 | 30.285 | −38.341 | 1.00 | 34.34 | C |
| ATOM | 496 | CB | LYS | | 77 | −17.096 | 29.536 | −37.706 | 1.00 | 35.97 | C |
| ATOM | 497 | CG | LYS | | 77 | −17.260 | 29.743 | −36.208 | 1.00 | 40.71 | C |
| ATOM | 498 | CD | LYS | | 77 | −17.760 | 31.143 | −35.876 | 1.00 | 43.03 | C |
| ATOM | 499 | CE | LYS | | 77 | −17.985 | 31.305 | −34.376 | 1.00 | 43.51 | C |
| ATOM | 500 | NZ | LYS | | 77 | −18.931 | 30.284 | −33.837 | 1.00 | 44.93 | N |
| ATOM | 501 | C | LYS | | 77 | −14.602 | 29.784 | −37.745 | 1.00 | 33.93 | C |
| ATOM | 502 | O | LYS | | 77 | −14.388 | 28.577 | −37.638 | 1.00 | 33.50 | O |
| ATOM | 503 | N | ILE | | 78 | −13.728 | 30.715 | −37.368 | 1.00 | 34.25 | N |
| ATOM | 504 | CA | ILE | | 78 | −12.437 | 30.373 | −36.764 | 1.00 | 34.01 | C |
| ATOM | 505 | CB | ILE | | 78 | −11.446 | 31.564 | −36.821 | 1.00 | 34.63 | C |
| ATOM | 506 | CG2 | ILE | | 78 | −10.088 | 31.135 | −36.269 | 1.00 | 33.43 | C |
| ATOM | 507 | CG1 | ILE | | 78 | −11.298 | 32.074 | −38.260 | 1.00 | 35.90 | C |
| ATOM | 508 | CD1 | ILE | | 78 | −10.411 | 31.231 | −39.142 | 1.00 | 35.86 | C |
| ATOM | 509 | C | ILE | | 78 | −12.688 | 30.046 | −35.289 | 1.00 | 33.77 | C |
| ATOM | 510 | O | ILE | | 78 | −13.233 | 30.869 | −34.559 | 1.00 | 33.99 | O |
| ATOM | 511 | N | THR | | 79 | −12.280 | 28.862 | −34.846 | 1.00 | 33.32 | N |
| ATOM | 512 | CA | THR | | 79 | −12.501 | 28.478 | −33.455 | 1.00 | 33.42 | C |
| ATOM | 513 | CB | THR | | 79 | −13.548 | 27.360 | −33.370 | 1.00 | 34.16 | C |
| ATOM | 514 | OG1 | THR | | 79 | −13.108 | 26.233 | −34.138 | 1.00 | 35.23 | O |
| ATOM | 515 | CG2 | THR | | 79 | −14.874 | 27.844 | −33.915 | 1.00 | 35.13 | C |
| ATOM | 516 | C | THR | | 79 | −11.257 | 28.029 | −32.685 | 1.00 | 32.98 | C |
| ATOM | 517 | O | THR | | 79 | −11.339 | 27.717 | −31.496 | 1.00 | 31.73 | O |
| ATOM | 518 | N | ARG | | 80 | −10.107 | 27.986 | −33.351 | 1.00 | 32.06 | N |
| ATOM | 519 | CA | ARG | | 80 | −8.883 | 27.570 | −32.671 | 1.00 | 29.59 | C |
| ATOM | 520 | CB | ARG | | 80 | −7.710 | 27.534 | −33.657 | 1.00 | 31.75 | C |
| ATOM | 521 | CG | ARG | | 80 | −7.267 | 28.907 | −34.132 | 1.00 | 32.62 | C |
| ATOM | 522 | CD | ARG | | 80 | −5.975 | 28.855 | −34.938 | 1.00 | 33.04 | C |
| ATOM | 523 | NE | ARG | | 80 | −6.207 | 28.717 | −36.372 | 1.00 | 37.11 | N |
| ATOM | 524 | CZ | ARG | | 80 | −6.361 | 27.562 | −37.005 | 1.00 | 37.45 | C |
| ATOM | 525 | NH1 | ARG | | 80 | −6.304 | 26.421 | −36.328 | 1.00 | 39.19 | N |
| ATOM | 526 | NH2 | ARG | | 80 | −6.580 | 27.552 | −38.317 | 1.00 | 35.65 | N |
| ATOM | 527 | C | ARG | | 80 | −8.584 | 28.555 | −31.546 | 1.00 | 27.69 | C |
| ATOM | 528 | O | ARG | | 80 | −8.962 | 29.722 | −31.620 | 1.00 | 27.39 | O |
| ATOM | 529 | N | THR | | 81 | −7.908 | 28.091 | −30.502 | 1.00 | 26.96 | N |
| ATOM | 530 | CA | THR | | 81 | −7.569 | 28.965 | −29.387 | 1.00 | 28.78 | C |
| ATOM | 531 | CB | THR | | 81 | −7.250 | 28.160 | −28.123 | 1.00 | 29.00 | C |
| ATOM | 532 | OG1 | THR | | 81 | −6.108 | 27.327 | −28.363 | 1.00 | 29.98 | O |
| ATOM | 533 | CG2 | THR | | 81 | −8.441 | 27.288 | −27.736 | 1.00 | 29.94 | C |
| ATOM | 534 | C | THR | | 81 | −6.338 | 29.782 | −29.749 | 1.00 | 29.24 | C |
| ATOM | 535 | O | THR | | 81 | −5.668 | 29.487 | −30.737 | 1.00 | 28.92 | O |
| ATOM | 536 | N | LYS | | 82 | −6.044 | 30.808 | −28.958 | 1.00 | 30.24 | N |
| ATOM | 537 | CA | LYS | | 82 | −4.864 | 31.635 | −29.200 | 1.00 | 30.05 | C |
| ATOM | 538 | CB | LYS | | 82 | −4.756 | 32.750 | −28.161 | 1.00 | 33.96 | C |
| ATOM | 539 | CG | LYS | | 82 | −5.863 | 33.780 | −28.210 | 1.00 | 39.25 | C |
| ATOM | 540 | CD | LYS | | 82 | −5.616 | 34.887 | −27.193 | 1.00 | 41.47 | C |
| ATOM | 541 | CE | LYS | | 82 | −5.531 | 34.333 | −25.778 | 1.00 | 44.01 | C |
| ATOM | 542 | NZ | LYS | | 82 | −5.354 | 35.413 | −24.770 | 1.00 | 44.71 | N |
| ATOM | 543 | C | LYS | | 82 | −3.621 | 30.751 | −29.096 | 1.00 | 29.66 | C |
| ATOM | 544 | O | LYS | | 82 | −2.681 | 30.877 | −29.884 | 1.00 | 25.85 | O |
| ATOM | 545 | N | GLU | | 83 | −3.628 | 29.861 | −28.108 | 1.00 | 28.55 | N |
| ATOM | 546 | CA | GLU | | 83 | −2.505 | 28.953 | −27.892 | 1.00 | 29.02 | C |
| ATOM | 547 | CB | GLU | | 83 | −2.760 | 28.075 | −26.665 | 1.00 | 31.20 | C |
| ATOM | 548 | CG | GLU | | 83 | −2.852 | 28.854 | −25.364 | 1.00 | 35.97 | C |
| ATOM | 549 | CD | GLU | | 83 | −4.156 | 29.626 | −25.218 | 1.00 | 37.98 | C |
| ATOM | 550 | OE1 | GLU | | 83 | −4.225 | 30.506 | −24.335 | 1.00 | 41.29 | O |
| ATOM | 551 | OE2 | GLU | | 83 | −5.111 | 29.350 | −25.971 | 1.00 | 37.74 | O |
| ATOM | 552 | C | GLU | | 83 | −2.252 | 28.070 | −29.108 | 1.00 | 26.59 | C |
| ATOM | 553 | O | GLU | | 83 | −1.104 | 27.875 | −29.511 | 1.00 | 24.73 | O |
| ATOM | 554 | N | GLU | | 84 | −3.321 | 27.531 | −29.685 | 1.00 | 25.40 | N |
| ATOM | 555 | CA | GLU | | 84 | −3.198 | 26.677 | −30.866 | 1.00 | 25.25 | C |
| ATOM | 556 | CB | GLU | | 84 | −4.553 | 26.049 | −31.223 | 1.00 | 26.42 | C |
| ATOM | 557 | CG | GLU | | 84 | −5.124 | 25.124 | −30.153 | 1.00 | 29.77 | C |
| ATOM | 558 | CD | GLU | | 84 | −6.481 | 24.547 | −30.539 | 1.00 | 31.27 | C |
| ATOM | 559 | OE1 | GLU | | 84 | −7.409 | 25.336 | −30.816 | 1.00 | 29.51 | O |
| ATOM | 560 | OE2 | GLU | | 84 | −6.618 | 23.306 | −30.564 | 1.00 | 33.67 | O |
| ATOM | 561 | C | GLU | | 84 | −2.686 | 27.500 | −32.052 | 1.00 | 25.07 | C |
| ATOM | 562 | O | GLU | | 84 | −1.875 | 27.024 | −32.849 | 1.00 | 24.45 | O |
| ATOM | 563 | N | ALA | | 85 | −3.163 | 28.735 | −32.160 | 1.00 | 23.80 | N |
| ATOM | 564 | CA | ALA | | 85 | −2.743 | 29.625 | −33.244 | 1.00 | 23.52 | C |
| ATOM | 565 | CB | ALA | | 85 | −3.535 | 30.926 | −33.188 | 1.00 | 23.72 | C |
| ATOM | 566 | C | ALA | | 85 | −1.244 | 29.920 | −33.146 | 1.00 | 23.46 | C |
| ATOM | 567 | O | ALA | | 85 | −0.544 | 29.994 | −34.161 | 1.00 | 21.39 | O |
| ATOM | 568 | N | LEU | | 86 | −0.755 | 30.097 | −31.922 | 1.00 | 22.67 | N |
| ATOM | 569 | CA | LEU | | 86 | 0.662 | 30.370 | −31.715 | 1.00 | 22.05 | C |
| ATOM | 570 | CB | LEU | | 86 | 0.919 | 30.763 | −30.258 | 1.00 | 22.26 | C |
| ATOM | 571 | CG | LEU | | 86 | 2.363 | 31.118 | −29.870 | 1.00 | 23.67 | C |
| ATOM | 572 | CD1 | LEU | | 86 | 2.868 | 32.269 | −30.740 | 1.00 | 22.88 | C |
| ATOM | 573 | CD2 | LEU | | 86 | 2.419 | 31.515 | −28.397 | 1.00 | 22.97 | C |
| ATOM | 574 | C | LEU | | 86 | 1.503 | 29.149 | −32.091 | 1.00 | 22.81 | C |
| ATOM | 575 | O | LEU | | 86 | 2.604 | 29.286 | −32.629 | 1.00 | 20.32 | O |
| ATOM | 576 | N | GLU | | 87 | 0.997 | 27.953 | −31.803 | 1.00 | 23.15 | N |
| ATOM | 577 | CA | GLU | | 87 | 1.741 | 26.747 | −32.144 | 1.00 | 25.76 | C |
| ATOM | 578 | CB | GLU | | 87 | 1.070 | 25.503 | −31.559 | 1.00 | 29.79 | C |
| ATOM | 579 | CG | GLU | | 87 | 1.276 | 25.379 | −30.062 | 1.00 | 35.40 | C |
| ATOM | 580 | CD | GLU | | 87 | 2.739 | 25.533 | −29.679 | 1.00 | 41.20 | C |
| ATOM | 581 | OE1 | GLU | | 87 | 3.564 | 24.709 | −30.131 | 1.00 | 41.60 | O |
| ATOM | 582 | OE2 | GLU | | 87 | 3.066 | 26.485 | −28.935 | 1.00 | 44.59 | O |
| ATOM | 583 | C | GLU | | 87 | 1.869 | 26.617 | −33.653 | 1.00 | 24.62 | C |
| ATOM | 584 | O | GLU | | 87 | 2.903 | 26.169 | −34.161 | 1.00 | 22.44 | O |
| ATOM | 585 | N | LEU | | 88 | 0.816 | 27.007 | −34.369 | 1.00 | 22.75 | N |
| ATOM | 586 | CA | LEU | | 88 | 0.826 | 26.955 | −35.829 | 1.00 | 21.50 | C |
| ATOM | 587 | CB | LEU | | 88 | −0.568 | 27.284 | −36.382 | 1.00 | 20.56 | C |
| ATOM | 588 | CG | LEU | | 88 | −1.572 | 26.129 | −36.310 | 1.00 | 23.77 | C |
| ATOM | 589 | CD1 | LEU | | 88 | −3.008 | 26.642 | −36.453 | 1.00 | 22.19 | C |
| ATOM | 590 | CD2 | LEU | | 88 | −1.232 | 25.118 | −37.401 | 1.00 | 22.20 | C |
| ATOM | 591 | C | LEU | | 88 | 1.858 | 27.949 | −36.363 | 1.00 | 19.42 | C |
| ATOM | 592 | O | LEU | | 88 | 2.651 | 27.617 | −37.252 | 1.00 | 19.73 | O |
| ATOM | 593 | N | ILE | | 89 | 1.838 | 29.159 | −35.809 | 1.00 | 18.57 | N |
| ATOM | 594 | CA | ILE | | 89 | 2.763 | 30.227 | −36.180 | 1.00 | 18.37 | C |
| ATOM | 595 | CB | ILE | | 89 | 2.475 | 31.508 | −35.349 | 1.00 | 18.61 | C |
| ATOM | 596 | CG2 | ILE | | 89 | 3.644 | 32.470 | −35.435 | 1.00 | 18.86 | C |
| ATOM | 597 | CG1 | ILE | | 89 | 1.188 | 32.189 | −35.842 | 1.00 | 17.15 | C |
| ATOM | 598 | CD1 | ILE | | 89 | 1.342 | 32.943 | −37.163 | 1.00 | 16.22 | C |
| ATOM | 599 | C | ILE | | 89 | 4.211 | 29.779 | −35.932 | 1.00 | 20.19 | C |
| ATOM | 600 | O | ILE | | 89 | 5.076 | 29.920 | −36.804 | 1.00 | 18.74 | O |
| ATOM | 601 | N | ASN | | 90 | 4.469 | 29.238 | −34.743 | 1.00 | 18.44 | N |
| ATOM | 602 | CA | ASN | | 90 | 5.813 | 28.785 | −34.409 | 1.00 | 19.72 | C |
| ATOM | 603 | CB | ASN | | 90 | 5.868 | 28.276 | −32.964 | 1.00 | 21.36 | C |
| ATOM | 604 | CG | ASN | | 90 | 5.759 | 29.396 | −31.947 | 1.00 | 22.39 | C |
| ATOM | 605 | OD1 | ASN | | 90 | 6.183 | 30.523 | −32.199 | 1.00 | 26.11 | O |
| ATOM | 606 | ND2 | ASN | | 90 | 5.209 | 29.083 | −30.781 | 1.00 | 26.54 | N |
| ATOM | 607 | C | ASN | | 90 | 6.300 | 27.691 | −35.356 | 1.00 | 19.73 | C |
| ATOM | 608 | O | ASN | | 90 | 7.490 | 27.608 | −35.644 | 1.00 | 19.99 | O |
| ATOM | 609 | N | GLY | | 91 | 5.381 | 26.857 | −35.835 | 1.00 | 19.30 | N |
| ATOM | 610 | CA | GLY | | 91 | 5.747 | 25.791 | −36.749 | 1.00 | 19.27 | C |
| ATOM | 611 | C | GLY | | 91 | 6.068 | 26.341 | −38.126 | 1.00 | 20.36 | C |
| ATOM | 612 | O | GLY | | 91 | 6.965 | 25.848 | −38.818 | 1.00 | 19.68 | O |
| ATOM | 613 | N | TYR | | 92 | 5.330 | 27.370 | −38.534 | 1.00 | 18.26 | N |
| ATOM | 614 | CA | TYR | | 92 | 5.569 | 27.991 | −39.830 | 1.00 | 17.78 | C |
| ATOM | 615 | CB | TYR | | 92 | 4.483 | 29.037 | −40.136 | 1.00 | 18.19 | C |
| ATOM | 616 | CG | TYR | | 92 | 3.107 | 28.437 | −40.361 | 1.00 | 19.32 | C |
| ATOM | 617 | CD1 | TYR | | 92 | 1.953 | 29.162 | −40.062 | 1.00 | 19.52 | C |
| ATOM | 618 | CE1 | TYR | | 92 | 0.684 | 28.618 | −40.266 | 1.00 | 20.24 | C |
| ATOM | 619 | CD2 | TYR | | 92 | 2.960 | 27.146 | −40.878 | 1.00 | 18.84 | C |
| ATOM | 620 | CE2 | TYR | | 92 | 1.695 | 26.592 | −41.087 | 1.00 | 21.13 | C |
| ATOM | 621 | CZ | TYR | | 92 | 0.559 | 27.338 | −40.776 | 1.00 | 20.32 | C |
| ATOM | 622 | OH | TYR | | 92 | −0.697 | 26.797 | −40.961 | 1.00 | 20.00 | O |
| ATOM | 623 | C | TYR | | 92 | 6.936 | 28.659 | −39.787 | 1.00 | 14.87 | C |
| ATOM | 624 | O | TYR | | 92 | 7.679 | 28.597 | −40.752 | 1.00 | 16.54 | O |
| ATOM | 625 | N | ILE | | 93 | 7.257 | 29.293 | −38.663 | 1.00 | 14.90 | N |
| ATOM | 626 | CA | ILE | | 93 | 8.545 | 29.967 | −38.500 | 1.00 | 17.95 | C |
| ATOM | 627 | CB | ILE | | 93 | 8.623 | 30.724 | −37.142 | 1.00 | 15.92 | C |
| ATOM | 628 | CG2 | ILE | | 93 | 10.071 | 31.164 | −36.852 | 1.00 | 16.05 | C |
| ATOM | 629 | CG1 | ILE | | 93 | 7.742 | 31.979 | −37.197 | 1.00 | 16.47 | C |
| ATOM | 630 | CD1 | ILE | | 93 | 7.580 | 32.684 | −35.839 | 1.00 | 16.42 | C |
| ATOM | 631 | C | ILE | | 93 | 9.697 | 28.966 | −38.602 | 1.00 | 17.73 | C |
| ATOM | 632 | O | ILE | | 93 | 10.721 | 29.249 | −39.234 | 1.00 | 16.34 | O |
| ATOM | 633 | N | GLN | | 94 | 9.531 | 27.797 | −37.993 | 1.00 | 18.12 | N |
| ATOM | 634 | CA | GLN | | 94 | 10.574 | 26.782 | −38.054 | 1.00 | 21.32 | C |
| ATOM | 635 | CB | GLN | | 94 | 10.217 | 25.579 | −37.173 | 1.00 | 23.22 | C |
| ATOM | 636 | CG | GLN | | 94 | 10.242 | 25.887 | −35.691 | 1.00 | 25.92 | C |
| ATOM | 637 | CD | GLN | | 94 | 10.036 | 24.654 | −34.836 | 1.00 | 31.08 | C |
| ATOM | 638 | OE1 | GLN | | 94 | 9.063 | 23.913 | −35.011 | 1.00 | 32.11 | O |
| ATOM | 639 | NE2 | GLN | | 94 | 10.949 | 24.428 | −33.899 | 1.00 | 31.37 | N |
| ATOM | 640 | C | GLN | | 94 | 10.813 | 26.317 | −39.487 | 1.00 | 21.15 | C |
| ATOM | 641 | O | GLN | | 94 | 11.959 | 26.177 | −39.911 | 1.00 | 22.23 | O |
| ATOM | 642 | N | LYS | | 95 | 9.739 | 26.086 | −40.236 | 1.00 | 20.91 | N |
| ATOM | 643 | CA | LYS | | 95 | 9.870 | 25.634 | −41.617 | 1.00 | 20.41 | C |
| ATOM | 644 | CB | LYS | | 95 | 8.511 | 25.205 | −42.173 | 1.00 | 24.55 | C |
| ATOM | 645 | CG | LYS | | 95 | 7.968 | 23.914 | −41.577 | 1.00 | 29.78 | C |
| ATOM | 646 | CD | LYS | | 95 | 6.720 | 23.454 | −42.324 | 1.00 | 33.95 | C |
| ATOM | 647 | CE | LYS | | 95 | 6.211 | 22.119 | −41.793 | 1.00 | 36.40 | C |
| ATOM | 648 | NZ | LYS | | 95 | 5.821 | 22.203 | −40.352 | 1.00 | 40.21 | N |
| ATOM | 649 | C | LYS | | 95 | 10.467 | 26.716 | −42.508 | 1.00 | 19.44 | C |
| ATOM | 650 | O | LYS | | 95 | 11.218 | 26.425 | −43.439 | 1.00 | 19.34 | O |
| ATOM | 651 | N | ILE | | 96 | 10.125 | 27.971 | −42.234 | 1.00 | 18.25 | N |
| ATOM | 652 | CA | ILE | | 96 | 10.659 | 29.070 | −43.020 | 1.00 | 17.52 | C |
| ATOM | 653 | CB | ILE | | 96 | 9.915 | 30.400 | −42.704 | 1.00 | 17.35 | C |
| ATOM | 654 | CG2 | ILE | | 96 | 10.606 | 31.580 | −43.412 | 1.00 | 15.30 | C |
| ATOM | 655 | CG1 | ILE | | 96 | 8.446 | 30.276 | −43.144 | 1.00 | 17.27 | C |
| ATOM | 656 | CD1 | ILE | | 96 | 7.558 | 31.470 | −42.767 | 1.00 | 18.12 | C |
| ATOM | 657 | C | ILE | | 96 | 12.153 | 29.228 | −42.734 | 1.00 | 16.25 | C |
| ATOM | 658 | O | ILE | | 96 | 12.956 | 29.408 | −43.654 | 1.00 | 17.72 | O |
| ATOM | 659 | N | LYS | | 97 | 12.529 | 29.135 | −41.462 | 1.00 | 15.88 | N |
| ATOM | 660 | CA | LYS | | 97 | 13.928 | 29.290 | −41.087 | 1.00 | 16.26 | C |
| ATOM | 661 | CB | LYS | | 97 | 14.066 | 29.463 | −39.570 | 1.00 | 15.39 | C |
| ATOM | 662 | CG | LYS | | 97 | 13.806 | 30.889 | −39.102 | 1.00 | 17.10 | C |
| ATOM | 663 | CD | LYS | | 97 | 14.218 | 31.066 | −37.662 | 1.00 | 15.69 | C |
| ATOM | 664 | CE | LYS | | 97 | 14.103 | 32.515 | −37.247 | 1.00 | 17.30 | C |
| ATOM | 665 | NZ | LYS | | 97 | 14.552 | 32.706 | −35.842 | 1.00 | 17.34 | N |
| ATOM | 666 | C | LYS | | 97 | 14.807 | 28.142 | −41.559 | 1.00 | 16.82 | C |
| ATOM | 667 | O | LYS | | 97 | 15.978 | 28.352 | −41.857 | 1.00 | 16.45 | O |
| ATOM | 668 | N | SER | | 98 | 14.249 | 26.937 | −41.620 | 1.00 | 17.13 | N |
| ATOM | 669 | CA | SER | | 98 | 15.005 | 25.770 | −42.086 | 1.00 | 19.37 | C |
| ATOM | 670 | CB | SER | | 98 | 14.311 | 24.485 | −41.647 | 1.00 | 18.74 | C |
| ATOM | 671 | OG | SER | | 98 | 13.081 | 24.351 | −42.335 | 1.00 | 18.36 | O |
| ATOM | 672 | C | SER | | 98 | 15.121 | 25.770 | −43.611 | 1.00 | 20.55 | C |
| ATOM | 673 | O | SER | | 98 | 16.011 | 25.127 | −44.185 | 1.00 | 19.81 | O |
| ATOM | 674 | N | GLY | | 99 | 14.222 | 26.496 | −44.272 | 1.00 | 19.70 | N |
| ATOM | 675 | CA | GLY | | 99 | 14.248 | 26.537 | −45.721 | 1.00 | 21.29 | C |
| ATOM | 676 | C | GLY | | 99 | 13.406 | 25.422 | −46.311 | 1.00 | 22.31 | C |
| ATOM | 677 | O | GLY | | 99 | 13.325 | 25.270 | −47.529 | 1.00 | 22.96 | O |
| ATOM | 678 | N | GLU | | 100 | 12.776 | 24.637 | −45.444 | 1.00 | 23.26 | N |
| ATOM | 679 | CA | GLU | | 100 | 11.920 | 23.542 | −45.888 | 1.00 | 26.21 | C |
| ATOM | 680 | CB | GLU | | 100 | 11.427 | 22.738 | −44.687 | 1.00 | 29.05 | C |
| ATOM | 681 | CG | GLU | | 100 | 12.530 | 21.992 | −43.959 | 1.00 | 37.13 | C |
| ATOM | 682 | CD | GLU | | 100 | 12.045 | 21.325 | −42.690 | 1.00 | 39.23 | C |
| ATOM | 683 | OE1 | GLU | | 100 | 11.630 | 22.046 | −41.759 | 1.00 | 41.16 | O |
| ATOM | 684 | OE2 | GLU | | 100 | 12.075 | 20.080 | −42.627 | 1.00 | 43.56 | O |
| ATOM | 685 | C | GLU | | 100 | 10.728 | 24.116 | −46.647 | 1.00 | 27.02 | C |
| ATOM | 686 | O | GLU | | 100 | 10.204 | 23.488 | −47.561 | 1.00 | 28.15 | O |
| ATOM | 687 | N | GLU | | 101 | 10.308 | 25.316 | −46.261 | 1.00 | 26.21 | N |
| ATOM | 688 | CA | GLU | | 101 | 9.188 | 25.998 | −46.907 | 1.00 | 28.61 | C |
| ATOM | 689 | CB | GLU | | 101 | 7.894 | 25.785 | −46.110 | 1.00 | 31.42 | C |
| ATOM | 690 | CG | GLU | | 101 | 7.207 | 24.457 | −46.362 | 1.00 | 34.52 | C |
| ATOM | 691 | CD | GLU | | 101 | 6.440 | 24.445 | −47.669 | 1.00 | 36.26 | C |
| ATOM | 692 | OE1 | GLU | | 101 | 7.039 | 24.748 | −48.723 | 1.00 | 39.55 | O |
| ATOM | 693 | OE2 | GLU | | 101 | 5.234 | 24.130 | −47.643 | 1.00 | 39.14 | O |
| ATOM | 694 | C | GLU | | 101 | 9.511 | 27.478 | −46.930 | 1.00 | 28.01 | C |
| ATOM | 695 | O | GLU | | 101 | 10.175 | 27.974 | −46.027 | 1.00 | 30.29 | O |
| ATOM | 696 | N | ASP | | 102 | 9.068 | 28.197 | −47.951 | 1.00 | 26.68 | N |
| ATOM | 697 | CA | ASP | | 102 | 9.355 | 29.618 | −47.949 | 1.00 | 27.54 | C |
| ATOM | 698 | CB | ASP | | 102 | 9.834 | 30.103 | −49.329 | 1.00 | 34.37 | C |
| ATOM | 699 | CG | ASP | | 102 | 8.865 | 29.786 | −50.441 | 1.00 | 37.59 | C |
| ATOM | 700 | OD1 | ASP | | 102 | 9.310 | 29.741 | −51.609 | 1.00 | 43.05 | O |
| ATOM | 701 | OD2 | ASP | | 102 | 7.668 | 29.594 | −50.157 | 1.00 | 41.47 | O |
| ATOM | 702 | C | ASP | | 102 | 8.138 | 30.387 | −47.462 | 1.00 | 25.40 | C |
| ATOM | 703 | O | ASP | | 102 | 7.011 | 29.890 | −47.487 | 1.00 | 23.71 | O |
| ATOM | 704 | N | PHE | | 103 | 8.391 | 31.588 | −46.974 | 1.00 | 23.34 | N |
| ATOM | 705 | CA | PHE | | 103 | 7.353 | 32.450 | −46.446 | 1.00 | 22.31 | C |
| ATOM | 706 | CB | PHE | | 103 | 7.955 | 33.825 | −46.158 | 1.00 | 22.80 | C |
| ATOM | 707 | CG | PHE | | 103 | 6.962 | 34.816 | −45.672 | 1.00 | 23.87 | C |
| ATOM | 708 | CD1 | PHE | | 103 | 6.597 | 34.850 | −44.335 | 1.00 | 24.41 | C |
| ATOM | 709 | CD2 | PHE | | 103 | 6.317 | 35.659 | −46.570 | 1.00 | 25.76 | C |
| ATOM | 710 | CE1 | PHE | | 103 | 5.595 | 35.702 | −43.897 | 1.00 | 24.83 | C |
| ATOM | 711 | CE2 | PHE | | 103 | 5.313 | 36.516 | −46.140 | 1.00 | 25.51 | C |
| ATOM | 712 | CZ | PHE | | 103 | 4.951 | 36.534 | −44.803 | 1.00 | 24.29 | C |
| ATOM | 713 | C | PHE | | 103 | 6.151 | 32.597 | −47.379 | 1.00 | 20.99 | C |
| ATOM | 714 | O | PHE | | 103 | 5.005 | 32.426 | −46.962 | 1.00 | 19.12 | O |
| ATOM | 715 | N | GLU | | 104 | 6.426 | 32.915 | −48.639 | 1.00 | 19.24 | N |
| ATOM | 716 | CA | GLU | | 104 | 5.388 | 33.123 | −49.645 | 1.00 | 21.00 | C |
| ATOM | 717 | CB | GLU | | 104 | 6.030 | 33.509 | −50.987 | 1.00 | 21.52 | C |
| ATOM | 718 | CG | GLU | | 104 | 6.796 | 34.828 | −50.940 | 1.00 | 22.25 | C |
| ATOM | 719 | CD | GLU | | 104 | 8.230 | 34.677 | −50.470 | 1.00 | 23.23 | C |
| ATOM | 720 | OE1 | GLU | | 104 | 8.557 | 33.677 | −49.789 | 1.00 | 23.13 | O |
| ATOM | 721 | OE2 | GLU | | 104 | 9.036 | 35.575 | −50.777 | 1.00 | 26.74 | O |
| ATOM | 722 | C | GLU | | 104 | 4.459 | 31.940 | −49.854 | 1.00 | 20.60 | C |
| ATOM | 723 | O | GLU | | 104 | 3.247 | 32.111 | −50.029 | 1.00 | 21.04 | O |
| ATOM | 724 | N | SER | | 105 | 5.023 | 30.742 | −49.851 | 1.00 | 20.47 | N |
| ATOM | 725 | CA | SER | | 105 | 4.226 | 29.542 | −50.049 | 1.00 | 22.89 | C |
| ATOM | 726 | CB | SER | | 105 | 5.133 | 28.328 | −50.232 | 1.00 | 23.63 | C |
| ATOM | 727 | OG | SER | | 105 | 4.368 | 27.149 | −50.326 | 1.00 | 25.68 | O |
| ATOM | 728 | C | SER | | 105 | 3.280 | 29.304 | −48.878 | 1.00 | 21.76 | C |
| ATOM | 729 | O | SER | | 105 | 2.095 | 29.027 | −49.078 | 1.00 | 22.05 | O |
| ATOM | 730 | N | LEU | | 106 | 3.801 | 29.406 | −47.660 | 1.00 | 20.34 | N |
| ATOM | 731 | CA | LEU | | 106 | 2.982 | 29.198 | −46.465 | 1.00 | 18.95 | C |
| ATOM | 732 | CB | LEU | | 106 | 3.861 | 29.178 | −45.208 | 1.00 | 16.50 | C |
| ATOM | 733 | CG | LEU | | 106 | 4.929 | 28.075 | −45.157 | 1.00 | 17.18 | C |
| ATOM | 734 | CD1 | LEU | | 106 | 5.640 | 28.119 | −43.799 | 1.00 | 15.09 | C |
| ATOM | 735 | CD2 | LEU | | 106 | 4.284 | 26.711 | −45.373 | 1.00 | 16.73 | C |
| ATOM | 736 | C | LEU | | 106 | 1.924 | 30.287 | −46.331 | 1.00 | 19.01 | C |
| ATOM | 737 | O | LEU | | 106 | 0.802 | 30.021 | −45.882 | 1.00 | 19.62 | O |
| ATOM | 738 | N | ALA | | 107 | 2.280 | 31.513 | −46.700 | 1.00 | 19.32 | N |
| ATOM | 739 | CA | ALA | | 107 | 1.330 | 32.618 | −46.629 | 1.00 | 20.46 | C |
| ATOM | 740 | CB | ALA | | 107 | 2.019 | 33.932 | −46.973 | 1.00 | 20.80 | C |
| ATOM | 741 | C | ALA | | 107 | 0.154 | 32.378 | −47.581 | 1.00 | 21.20 | C |
| ATOM | 742 | O | ALA | | 107 | −1.013 | 32.494 | −47.184 | 1.00 | 20.09 | O |
| ATOM | 743 | N | SER | | 108 | 0.458 | 32.042 | −48.834 | 1.00 | 19.42 | N |
| ATOM | 744 | CA | SER | | 108 | −0.586 | 31.798 | −49.827 | 1.00 | 20.67 | C |
| ATOM | 745 | CB | SER | | 108 | 0.038 | 31.430 | −51.179 | 1.00 | 21.16 | C |
| ATOM | 746 | OG | SER | | 108 | 0.864 | 32.479 | −51.658 | 1.00 | 26.10 | O |
| ATOM | 747 | C | SER | | 108 | −1.505 | 30.667 | −49.380 | 1.00 | 21.18 | C |
| ATOM | 748 | O | SER | | 108 | −2.707 | 30.671 | −49.654 | 1.00 | 21.09 | O |
| ATOM | 749 | N | GLN | | 109 | −0.931 | 29.705 | −48.671 | 1.00 | 20.49 | N |
| ATOM | 750 | CA | GLN | | 109 | −1.681 | 28.551 | −48.214 | 1.00 | 22.48 | C |
| ATOM | 751 | CB | GLN | | 109 | −0.750 | 27.351 | −48.088 | 1.00 | 22.52 | C |
| ATOM | 752 | CG | GLN | | 109 | −0.254 | 26.790 | −49.392 | 1.00 | 23.08 | C |
| ATOM | 753 | CD | GLN | | 109 | 0.724 | 25.666 | −49.166 | 1.00 | 25.92 | C |
| ATOM | 754 | OE1 | GLN | | 109 | 1.930 | 25.888 | −49.042 | 1.00 | 26.77 | O |
| ATOM | 755 | NE2 | GLN | | 109 | 0.209 | 24.450 | −49.090 | 1.00 | 24.41 | N |
| ATOM | 756 | C | GLN | | 109 | −2.427 | 28.683 | −46.900 | 1.00 | 21.83 | C |
| ATOM | 757 | O | GLN | | 109 | −3.587 | 28.311 | −46.801 | 1.00 | 20.58 | O |
| ATOM | 758 | N | PHE | | 110 | −1.757 | 29.221 | −45.892 | 1.00 | 21.69 | N |
| ATOM | 759 | CA | PHE | | 110 | −2.353 | 29.274 | −44.574 | 1.00 | 22.41 | C |
| ATOM | 760 | CB | PHE | | 110 | −1.471 | 28.442 | −43.645 | 1.00 | 21.55 | C |
| ATOM | 761 | CG | PHE | | 110 | −1.139 | 27.080 | −44.201 | 1.00 | 21.62 | C |
| ATOM | 762 | CD1 | PHE | | 110 | 0.174 | 26.732 | −44.499 | 1.00 | 21.24 | C |
| ATOM | 763 | CD2 | PHE | | 110 | −2.149 | 26.157 | −44.455 | 1.00 | 21.95 | C |
| ATOM | 764 | CE1 | PHE | | 110 | 0.474 | 25.485 | −45.044 | 1.00 | 24.45 | C |
| ATOM | 765 | CE2 | PHE | | 110 | −1.864 | 24.912 | −44.996 | 1.00 | 22.45 | C |
| ATOM | 766 | CZ | PHE | | 110 | −0.551 | 24.571 | −45.294 | 1.00 | 24.44 | C |
| ATOM | 767 | C | PHE | | 110 | −2.687 | 30.606 | −43.928 | 1.00 | 22.56 | C |
| ATOM | 768 | O | PHE | | 110 | −3.248 | 30.628 | −42.839 | 1.00 | 23.57 | O |
| ATOM | 769 | N | SER | | 111 | −2.367 | 31.719 | −44.571 | 1.00 | 21.02 | N |
| ATOM | 770 | CA | SER | | 111 | −2.697 | 32.997 | −43.961 | 1.00 | 21.58 | C |
| ATOM | 771 | CB | SER | | 111 | −2.030 | 34.146 | −44.691 | 1.00 | 18.53 | C |
| ATOM | 772 | OG | SER | | 111 | −2.347 | 35.369 | −44.036 | 1.00 | 20.21 | O |
| ATOM | 773 | C | SER | | 111 | −4.206 | 33.242 | −43.951 | 1.00 | 22.85 | C |
| ATOM | 774 | O | SER | | 111 | −4.876 | 33.071 | −44.975 | 1.00 | 22.36 | O |
| ATOM | 775 | N | ASP | | 112 | −4.722 | 33.648 | −42.793 | 1.00 | 23.38 | N |
| ATOM | 776 | CA | ASP | | 112 | −6.145 | 33.937 | −42.619 | 1.00 | 25.80 | C |
| ATOM | 777 | CB | ASP | | 112 | −6.578 | 33.724 | −41.159 | 1.00 | 24.20 | C |
| ATOM | 778 | CG | ASP | | 112 | −6.822 | 32.261 | −40.820 | 1.00 | 26.33 | C |
| ATOM | 779 | OD1 | ASP | | 112 | −7.520 | 31.578 | −41.599 | 1.00 | 26.12 | O |
| ATOM | 780 | OD2 | ASP | | 112 | −6.333 | 31.789 | −39.767 | 1.00 | 23.37 | O |
| ATOM | 781 | C | ASP | | 112 | −6.436 | 35.379 | −43.008 | 1.00 | 25.72 | C |
| ATOM | 782 | O | ASP | | 112 | −7.283 | 36.030 | −42.406 | 1.00 | 28.16 | O |
| ATOM | 783 | N | CYS | | 113 | −5.723 | 35.884 | −44.004 | 1.00 | 26.38 | N |
| ATOM | 784 | CA | CYS | | 113 | −5.914 | 37.252 | −44.458 | 1.00 | 25.54 | C |
| ATOM | 785 | CB | CYS | | 113 | −4.672 | 38.090 | −44.141 | 1.00 | 24.34 | C |
| ATOM | 786 | SG | CYS | | 113 | −4.765 | 39.850 | −44.586 | 1.00 | 26.84 | S |
| ATOM | 787 | C | CYS | | 113 | −6.154 | 37.234 | −45.954 | 1.00 | 25.63 | C |
| ATOM | 788 | O | CYS | | 113 | −5.685 | 36.337 | −46.651 | 1.00 | 25.42 | O |
| ATOM | 789 | N | SER | | 114 | −6.891 | 38.224 | −46.444 | 1.00 | 24.84 | N |
| ATOM | 790 | CA | SER | | 114 | −7.181 | 38.321 | −47.870 | 1.00 | 25.86 | C |
| ATOM | 791 | CB | SER | | 114 | −8.167 | 39.469 | −48.132 | 1.00 | 26.75 | C |
| ATOM | 792 | OG | SER | | 114 | −7.695 | 40.692 | −47.593 | 1.00 | 29.35 | O |
| ATOM | 793 | C | SER | | 114 | −5.893 | 38.542 | −48.667 | 1.00 | 25.32 | C |
| ATOM | 794 | O | SER | | 114 | −5.840 | 38.261 | −49.872 | 1.00 | 24.77 | O |
| ATOM | 795 | N | SER | | 115 | −4.854 | 39.033 | −47.995 | 1.00 | 23.19 | N |
| ATOM | 796 | CA | SER | | 115 | −3.572 | 39.278 | −48.664 | 1.00 | 22.81 | C |
| ATOM | 797 | CB | SER | | 115 | −2.665 | 40.159 | −47.798 | 1.00 | 20.33 | C |
| ATOM | 798 | OG | SER | | 115 | −2.422 | 39.568 | −46.531 | 1.00 | 20.91 | O |
| ATOM | 799 | C | SER | | 115 | −2.849 | 37.975 | −49.001 | 1.00 | 21.09 | C |
| ATOM | 800 | O | SER | | 115 | −1.799 | 37.989 | −49.640 | 1.00 | 20.29 | O |
| ATOM | 801 | N | ALA | | 116 | −3.412 | 36.849 | −48.574 | 1.00 | 20.25 | N |
| ATOM | 802 | CA | ALA | | 116 | −2.824 | 35.546 | −48.855 | 1.00 | 19.33 | C |
| ATOM | 803 | CB | ALA | | 116 | −3.664 | 34.441 | −48.217 | 1.00 | 22.64 | C |
| ATOM | 804 | C | ALA | | 116 | −2.750 | 35.329 | −50.368 | 1.00 | 21.09 | C |
| ATOM | 805 | O | ALA | | 116 | −1.811 | 34.707 | −50.870 | 1.00 | 17.16 | O |
| ATOM | 806 | N | LYS | | 117 | −3.747 | 35.845 | −51.088 | 1.00 | 18.87 | N |
| ATOM | 807 | CA | LYS | | 117 | −3.806 | 35.699 | −52.542 | 1.00 | 20.20 | C |
| ATOM | 808 | CB | LYS | | 117 | −5.134 | 36.257 | −53.068 | 1.00 | 23.03 | C |
| ATOM | 809 | CG | LYS | | 117 | −6.366 | 35.604 | −52.451 | 1.00 | 29.17 | C |
| ATOM | 810 | CD | LYS | | 117 | −6.627 | 34.220 | −53.029 | 1.00 | 31.39 | C |
| ATOM | 811 | CE | LYS | | 117 | −7.717 | 33.479 | −52.245 | 1.00 | 34.23 | C |
| ATOM | 812 | NZ | LYS | | 117 | −7.223 | 33.034 | −50.901 | 1.00 | 32.34 | N |
| ATOM | 813 | C | LYS | | 117 | −2.640 | 36.404 | −53.228 | 1.00 | 19.22 | C |
| ATOM | 814 | O | LYS | | 117 | −2.336 | 36.131 | −54.392 | 1.00 | 17.70 | O |
| ATOM | 815 | N | ALA | | 118 | −1.999 | 37.319 | −52.508 | 1.00 | 17.95 | N |
| ATOM | 816 | CA | ALA | | 118 | −0.854 | 38.045 | −53.038 | 1.00 | 19.01 | C |
| ATOM | 817 | CB | ALA | | 118 | −1.085 | 39.551 | −52.930 | 1.00 | 17.73 | C |
| ATOM | 818 | C | ALA | | 118 | 0.411 | 37.643 | −52.272 | 1.00 | 19.64 | C |
| ATOM | 819 | O | ALA | | 118 | 1.288 | 38.470 | −52.015 | 1.00 | 19.72 | O |
| ATOM | 820 | N | ARG | | 119 | 0.489 | 36.366 | −51.915 | 1.00 | 18.61 | N |
| ATOM | 821 | CA | ARG | | 119 | 1.634 | 35.825 | −51.189 | 1.00 | 21.48 | C |
| ATOM | 822 | CB | ARG | | 119 | 2.889 | 35.863 | −52.079 | 1.00 | 24.72 | C |
| ATOM | 823 | CG | ARG | | 119 | 2.947 | 34.743 | −53.119 | 1.00 | 27.85 | C |
| ATOM | 824 | CD | ARG | | 119 | 3.738 | 35.130 | −54.367 | 1.00 | 31.89 | C |
| ATOM | 825 | NE | ARG | | 119 | 2.901 | 35.895 | −55.290 | 1.00 | 38.23 | N |
| ATOM | 826 | CZ | ARG | | 119 | 2.579 | 37.180 | −55.144 | 1.00 | 38.28 | C |
| ATOM | 827 | NH1 | ARG | | 119 | 3.033 | 37.886 | −54.112 | 1.00 | 39.89 | N |
| ATOM | 828 | NH2 | ARG | | 119 | 1.763 | 37.753 | −56.016 | 1.00 | 33.97 | N |
| ATOM | 829 | C | ARG | | 119 | 1.881 | 36.589 | −49.893 | 1.00 | 20.62 | C |
| ATOM | 830 | O | ARG | | 119 | 3.008 | 36.656 | −49.407 | 1.00 | 18.56 | O |
| ATOM | 831 | N | GLY | | 120 | 0.817 | 37.171 | −49.343 | 1.00 | 20.89 | N |
| ATOM | 832 | CA | GLY | | 120 | 0.934 | 37.908 | −48.094 | 1.00 | 21.30 | C |
| ATOM | 833 | C | GLY | | 120 | 1.363 | 39.359 | −48.201 | 1.00 | 22.05 | C |
| ATOM | 834 | O | GLY | | 120 | 1.426 | 40.053 | −47.191 | 1.00 | 21.34 | O |
| ATOM | 835 | N | ASP | | 121 | 1.640 | 39.832 | −49.414 | 1.00 | 22.44 | N |
| ATOM | 836 | CA | ASP | | 121 | 2.073 | 41.214 | −49.608 | 1.00 | 22.42 | C |
| ATOM | 837 | CB | ASP | | 121 | 2.534 | 41.429 | −51.053 | 1.00 | 22.20 | C |
| ATOM | 838 | CG | ASP | | 121 | 2.921 | 42.870 | −51.334 | 1.00 | 23.17 | C |
| ATOM | 839 | OD1 | ASP | | 121 | 3.600 | 43.489 | −50.487 | 1.00 | 21.32 | O |
| ATOM | 840 | OD2 | ASP | | 121 | 2.553 | 43.381 | −52.410 | 1.00 | 22.33 | O |
| ATOM | 841 | C | ASP | | 121 | 0.990 | 42.232 | −49.262 | 1.00 | 24.01 | C |
| ATOM | 842 | O | ASP | | 121 | −0.141 | 42.132 | −49.728 | 1.00 | 22.67 | O |
| ATOM | 843 | N | LEU | | 122 | 1.363 | 43.219 | −48.450 | 1.00 | 24.20 | N |
| ATOM | 844 | CA | LEU | | 122 | 0.449 | 44.263 | −48.017 | 1.00 | 25.71 | C |
| ATOM | 845 | CB | LEU | | 122 | 0.580 | 44.482 | −46.510 | 1.00 | 24.03 | C |
| ATOM | 846 | CG | LEU | | 122 | 0.179 | 43.321 | −45.610 | 1.00 | 24.14 | C |
| ATOM | 847 | CD1 | LEU | | 122 | 0.457 | 43.698 | −44.155 | 1.00 | 22.74 | C |
| ATOM | 848 | CD2 | LEU | | 122 | −1.294 | 43.007 | −45.818 | 1.00 | 23.98 | C |
| ATOM | 849 | C | LEU | | 122 | 0.701 | 45.589 | −48.710 | 1.00 | 26.77 | C |
| ATOM | 850 | O | LEU | | 122 | −0.069 | 46.530 | −48.541 | 1.00 | 27.12 | O |
| ATOM | 851 | N | GLY | | 123 | 1.777 | 45.666 | −49.481 | 1.00 | 28.17 | N |
| ATOM | 852 | CA | GLY | | 123 | 2.101 | 46.913 | −50.144 | 1.00 | 28.92 | C |
| ATOM | 853 | C | GLY | | 123 | 2.829 | 47.781 | −49.138 | 1.00 | 30.04 | C |
| ATOM | 854 | O | GLY | | 123 | 3.054 | 47.356 | −48.003 | 1.00 | 30.87 | O |
| ATOM | 855 | N | ALA | | 124 | 3.198 | 48.992 | −49.537 | 1.00 | 28.94 | N |
| ATOM | 856 | CA | ALA | | 124 | 3.910 | 49.892 | −48.641 | 1.00 | 30.19 | C |
| ATOM | 857 | CB | ALA | | 124 | 4.740 | 50.886 | −49.451 | 1.00 | 30.74 | C |
| ATOM | 858 | C | ALA | | 124 | 2.928 | 50.635 | −47.746 | 1.00 | 31.41 | C |
| ATOM | 859 | O | ALA | | 124 | 1.744 | 50.747 | −48.064 | 1.00 | 31.90 | O |
| ATOM | 860 | N | PHE | | 125 | 3.419 | 51.128 | −46.615 | 1.00 | 31.96 | N |
| ATOM | 861 | CA | PHE | | 125 | 2.576 | 51.868 | −45.683 | 1.00 | 32.00 | C |
| ATOM | 862 | CB | PHE | | 125 | 1.660 | 50.907 | −44.905 | 1.00 | 32.14 | C |
| ATOM | 863 | CG | PHE | | 125 | 2.394 | 49.832 | −44.132 | 1.00 | 32.47 | C |
| ATOM | 864 | CD1 | PHE | | 125 | 3.038 | 50.126 | −42.932 | 1.00 | 32.28 | C |
| ATOM | 865 | CD2 | PHE | | 125 | 2.421 | 48.519 | −44.601 | 1.00 | 32.76 | C |
| ATOM | 866 | CE1 | PHE | | 125 | 3.697 | 49.131 | −42.206 | 1.00 | 31.38 | C |
| ATOM | 867 | CE2 | PHE | | 125 | 3.077 | 47.514 | −43.885 | 1.00 | 32.65 | C |
| ATOM | 868 | CZ | PHE | | 125 | 3.717 | 47.822 | −42.681 | 1.00 | 30.91 | C |
| ATOM | 869 | C | PHE | | 125 | 3.406 | 52.709 | −44.726 | 1.00 | 31.21 | C |
| ATOM | 870 | O | PHE | | 125 | 4.608 | 52.498 | −44.578 | 1.00 | 32.99 | O |
| ATOM | 871 | N | SER | | 126 | 2.765 | 53.677 | −44.087 | 1.00 | 32.12 | N |
| ATOM | 872 | CA | SER | | 126 | 3.460 | 54.540 | −43.145 | 1.00 | 33.01 | C |
| ATOM | 873 | CB | SER | | 126 | 3.303 | 56.008 | −43.551 | 1.00 | 34.29 | C |
| ATOM | 874 | OG | SER | | 126 | 1.941 | 56.376 | −43.561 | 1.00 | 35.69 | O |
| ATOM | 875 | C | SER | | 126 | 2.885 | 54.315 | −41.755 | 1.00 | 31.94 | C |
| ATOM | 876 | O | SER | | 126 | 1.956 | 53.524 | −41.579 | 1.00 | 31.18 | O |
| ATOM | 877 | N | ARG | | 127 | 3.439 | 55.003 | −40.766 | 1.00 | 33.72 | N |
| ATOM | 878 | CA | ARG | | 127 | 2.957 | 54.855 | −39.404 | 1.00 | 34.85 | C |
| ATOM | 879 | CB | ARG | | 127 | 3.920 | 55.528 | −38.424 | 1.00 | 34.40 | C |
| ATOM | 880 | CG | ARG | | 127 | 5.199 | 54.736 | −38.160 | 1.00 | 33.60 | C |
| ATOM | 881 | CD | ARG | | 127 | 5.993 | 55.353 | −37.018 | 1.00 | 32.21 | C |
| ATOM | 882 | NE | ARG | | 127 | 7.195 | 54.598 | −36.658 | 1.00 | 31.62 | N |
| ATOM | 883 | CZ | ARG | | 127 | 7.211 | 53.507 | −35.893 | 1.00 | 30.76 | C |
| ATOM | 884 | NH1 | ARG | | 127 | 6.085 | 53.018 | −35.396 | 1.00 | 27.78 | N |
| ATOM | 885 | NH2 | ARG | | 127 | 8.364 | 52.916 | −35.604 | 1.00 | 30.85 | N |
| ATOM | 886 | C | ARG | | 127 | 1.553 | 55.428 | −39.238 | 1.00 | 37.37 | C |
| ATOM | 887 | O | ARG | | 127 | 1.233 | 56.485 | −39.787 | 1.00 | 37.57 | O |
| ATOM | 888 | N | GLY | | 128 | 0.716 | 54.713 | −38.494 | 1.00 | 38.78 | N |
| ATOM | 889 | CA | GLY | | 128 | −0.641 | 55.166 | −38.253 | 1.00 | 40.45 | C |
| ATOM | 890 | C | GLY | | 128 | −1.716 | 54.449 | −39.047 | 1.00 | 40.51 | C |
| ATOM | 891 | O | GLY | | 128 | −2.903 | 54.663 | −38.811 | 1.00 | 42.58 | O |
| ATOM | 892 | N | GLN | | 129 | −1.313 | 53.592 | −39.979 | 1.00 | 38.75 | N |
| ATOM | 893 | CA | GLN | | 129 | −2.272 | 52.871 | −40.807 | 1.00 | 37.32 | C |
| ATOM | 894 | CB | GLN | | 129 | −1.742 | 52.754 | −42.238 | 1.00 | 39.24 | C |
| ATOM | 895 | CG | GLN | | 129 | −1.226 | 54.057 | −42.833 | 1.00 | 41.99 | C |
| ATOM | 896 | CD | GLN | | 129 | −0.708 | 53.885 | −44.256 | 1.00 | 43.29 | C |
| ATOM | 897 | OE1 | GLN | | 129 | −0.117 | 54.799 | −44.826 | 1.00 | 44.89 | O |
| ATOM | 898 | NE2 | GLN | | 129 | −0.934 | 52.711 | −44.832 | 1.00 | 45.91 | N |
| ATOM | 899 | C | GLN | | 129 | −2.583 | 51.474 | −40.278 | 1.00 | 36.07 | C |
| ATOM | 900 | O | GLN | | 129 | −3.736 | 51.044 | −40.269 | 1.00 | 36.17 | O |
| ATOM | 901 | N | MET | | 130 | −1.552 | 50.761 | −39.839 | 1.00 | 33.33 | N |
| ATOM | 902 | CA | MET | | 130 | −1.741 | 49.407 | −39.340 | 1.00 | 32.52 | C |
| ATOM | 903 | CB | MET | | 130 | −0.554 | 48.532 | −39.760 | 1.00 | 31.71 | C |
| ATOM | 904 | CG | MET | | 130 | −0.244 | 48.572 | −41.249 | 1.00 | 33.63 | C |
| ATOM | 905 | SD | MET | | 130 | −1.533 | 47.832 | −42.264 | 1.00 | 36.73 | S |
| ATOM | 906 | CE | MET | | 130 | −0.783 | 47.898 | −43.882 | 1.00 | 36.14 | C |
| ATOM | 907 | C | MET | | 130 | −1.871 | 49.390 | −37.823 | 1.00 | 31.76 | C |
| ATOM | 908 | O | MET | | 130 | −1.551 | 50.373 | −37.157 | 1.00 | 31.56 | O |
| ATOM | 909 | N | GLN | | 131 | −2.348 | 48.274 | −37.279 | 1.00 | 31.61 | N |
| ATOM | 910 | CA | GLN | | 131 | −2.474 | 48.154 | −35.834 | 1.00 | 32.12 | C |
| ATOM | 911 | CB | GLN | | 131 | −3.005 | 46.777 | −35.471 | 1.00 | 34.43 | C |
| ATOM | 912 | CG | GLN | | 131 | −4.400 | 46.572 | −36.018 | 1.00 | 37.18 | C |
| ATOM | 913 | CD | GLN | | 131 | −4.967 | 45.226 | −35.680 | 1.00 | 38.83 | C |
| ATOM | 914 | OE1 | GLN | | 131 | −5.108 | 44.877 | −34.510 | 1.00 | 43.14 | O |
| ATOM | 915 | NE2 | GLN | | 131 | −5.298 | 44.455 | −36.703 | 1.00 | 39.26 | N |
| ATOM | 916 | C | GLN | | 131 | −1.093 | 48.402 | −35.249 | 1.00 | 31.69 | C |
| ATOM | 917 | O | GLN | | 131 | −0.080 | 47.971 | −35.807 | 1.00 | 31.12 | O |
| ATOM | 918 | N | LYS | | 132 | −1.061 | 49.113 | −34.130 | 1.00 | 28.74 | N |
| ATOM | 919 | CA | LYS | | 132 | 0.183 | 49.499 | −33.492 | 1.00 | 26.91 | C |
| ATOM | 920 | CB | LYS | | 132 | −0.123 | 50.136 | −32.133 | 1.00 | 27.76 | C |
| ATOM | 921 | CG | LYS | | 132 | 1.037 | 50.907 | −31.535 | 1.00 | 27.78 | C |
| ATOM | 922 | CD | LYS | | 132 | 1.653 | 51.862 | −32.532 | 1.00 | 26.57 | C |
| ATOM | 923 | CE | LYS | | 132 | 2.829 | 52.600 | −31.910 | 1.00 | 27.44 | C |
| ATOM | 924 | NZ | LYS | | 132 | 3.657 | 53.293 | −32.928 | 1.00 | 28.79 | N |
| ATOM | 925 | C | LYS | | 132 | 1.273 | 48.434 | −33.342 | 1.00 | 26.22 | C |
| ATOM | 926 | O | LYS | | 132 | 2.423 | 48.679 | −33.707 | 1.00 | 25.72 | O |
| ATOM | 927 | N | PRO | | 133 | 0.936 | 47.254 | −32.797 | 1.00 | 24.78 | N |
| ATOM | 928 | CD | PRO | | 133 | −0.361 | 46.831 | −32.242 | 1.00 | 26.39 | C |
| ATOM | 929 | CA | PRO | | 133 | 1.944 | 46.198 | −32.628 | 1.00 | 26.25 | C |
| ATOM | 930 | CB | PRO | | 133 | 1.148 | 45.060 | −31.990 | 1.00 | 27.61 | C |
| ATOM | 931 | CG | PRO | | 133 | 0.055 | 45.778 | −31.247 | 1.00 | 27.91 | C |
| ATOM | 932 | C | PRO | | 133 | 2.573 | 45.782 | −33.959 | 1.00 | 25.91 | C |
| ATOM | 933 | O | PRO | | 133 | 3.787 | 45.566 | −34.048 | 1.00 | 25.34 | O |
| ATOM | 934 | N | PHE | | 134 | 1.733 | 45.676 | −34.984 | 1.00 | 25.36 | N |
| ATOM | 935 | CA | PHE | | 134 | 2.172 | 45.288 | −36.326 | 1.00 | 23.87 | C |
| ATOM | 936 | CB | PHE | | 134 | 0.950 | 45.094 | −37.225 | 1.00 | 22.16 | C |
| ATOM | 937 | CG | PHE | | 134 | 1.269 | 44.499 | −38.570 | 1.00 | 20.35 | C |
| ATOM | 938 | CD1 | PHE | | 134 | 1.143 | 43.131 | −38.783 | 1.00 | 18.13 | C |
| ATOM | 939 | CD2 | PHE | | 134 | 1.691 | 45.307 | −39.618 | 1.00 | 19.91 | C |
| ATOM | 940 | CE1 | PHE | | 134 | 1.433 | 42.567 | −40.030 | 1.00 | 19.24 | C |
| ATOM | 941 | CE2 | PHE | | 134 | 1.985 | 44.758 | −40.870 | 1.00 | 18.90 | C |
| ATOM | 942 | CZ | PHE | | 134 | 1.855 | 43.383 | −41.075 | 1.00 | 19.12 | C |
| ATOM | 943 | C | PHE | | 134 | 3.058 | 46.389 | −36.898 | 1.00 | 23.25 | C |
| ATOM | 944 | O | PHE | | 134 | 4.102 | 46.128 | −37.502 | 1.00 | 22.16 | O |
| ATOM | 945 | N | GLU | | 135 | 2.636 | 47.634 | −36.698 | 1.00 | 23.54 | N |
| ATOM | 946 | CA | GLU | | 135 | 3.391 | 48.778 | −37.188 | 1.00 | 22.32 | C |
| ATOM | 947 | CB | GLU | | 135 | 2.626 | 50.067 | −36.890 | 1.00 | 23.65 | C |
| ATOM | 948 | CG | GLU | | 135 | 3.431 | 51.324 | −37.128 | 1.00 | 25.84 | C |
| ATOM | 949 | CD | GLU | | 135 | 2.699 | 52.563 | −36.657 | 1.00 | 28.66 | C |
| ATOM | 950 | OE1 | GLU | | 135 | 1.533 | 52.743 | −37.064 | 1.00 | 28.63 | O |
| ATOM | 951 | OE2 | GLU | | 135 | 3.290 | 53.346 | −35.883 | 1.00 | 30.94 | O |
| ATOM | 952 | C | GLU | | 135 | 4.794 | 48.859 | −36.585 | 1.00 | 22.06 | C |
| ATOM | 953 | O | GLU | | 135 | 5.792 | 48.958 | −37.313 | 1.00 | 19.26 | O |
| ATOM | 954 | N | ASP | | 136 | 4.877 | 48.829 | −35.257 | 1.00 | 21.97 | N |
| ATOM | 955 | CA | ASP | | 136 | 6.176 | 48.913 | −34.590 | 1.00 | 21.65 | C |
| ATOM | 956 | CB | ASP | | 136 | 6.016 | 48.909 | −33.064 | 1.00 | 24.59 | C |
| ATOM | 957 | CG | ASP | | 136 | 5.397 | 50.196 | −32.538 | 1.00 | 24.60 | C |
| ATOM | 958 | OD1 | ASP | | 136 | 5.452 | 51.217 | −33.251 | 1.00 | 24.55 | O |
| ATOM | 959 | OD2 | ASP | | 136 | 4.866 | 50.188 | −31.411 | 1.00 | 25.54 | O |
| ATOM | 960 | C | ASP | | 136 | 7.094 | 47.781 | −35.010 | 1.00 | 20.28 | C |
| ATOM | 961 | O | ASP | | 136 | 8.282 | 47.997 | −35.243 | 1.00 | 20.40 | O |
| ATOM | 962 | N | ALA | | 137 | 6.547 | 46.576 | −35.114 | 1.00 | 20.56 | N |
| ATOM | 963 | CA | ALA | | 137 | 7.347 | 45.430 | −35.534 | 1.00 | 20.52 | C |
| ATOM | 964 | CB | ALA | | 137 | 6.503 | 44.150 | −35.480 | 1.00 | 20.41 | C |
| ATOM | 965 | C | ALA | | 137 | 7.855 | 45.675 | −36.961 | 1.00 | 20.52 | C |
| ATOM | 966 | O | ALA | | 137 | 9.034 | 45.483 | −37.254 | 1.00 | 19.91 | O |
| ATOM | 967 | N | SER | | 138 | 6.966 | 46.132 | −37.838 | 1.00 | 20.50 | N |
| ATOM | 968 | CA | SER | | 138 | 7.332 | 46.392 | −39.229 | 1.00 | 21.18 | C |
| ATOM | 969 | CB | SER | | 138 | 6.121 | 46.885 | −40.028 | 1.00 | 20.16 | C |
| ATOM | 970 | OG | SER | | 138 | 5.124 | 45.886 | −40.118 | 1.00 | 18.18 | O |
| ATOM | 971 | C | SER | | 138 | 8.459 | 47.396 | −39.381 | 1.00 | 22.52 | C |
| ATOM | 972 | O | SER | | 138 | 9.391 | 47.164 | −40.152 | 1.00 | 21.15 | O |
| ATOM | 973 | N | PHE | | 139 | 8.371 | 48.511 | −38.654 | 1.00 | 22.86 | N |
| ATOM | 974 | CA | PHE | | 139 | 9.386 | 49.559 | −38.726 | 1.00 | 24.53 | C |
| ATOM | 975 | CB | PHE | | 139 | 8.806 | 50.906 | −38.272 | 1.00 | 24.41 | C |
| ATOM | 976 | CG | PHE | | 139 | 7.967 | 51.582 | −39.320 | 1.00 | 24.60 | C |
| ATOM | 977 | CD1 | PHE | | 139 | 6.624 | 51.264 | −39.471 | 1.00 | 24.06 | C |
| ATOM | 978 | CD2 | PHE | | 139 | 8.541 | 52.503 | −40.195 | 1.00 | 26.09 | C |
| ATOM | 979 | CE1 | PHE | | 139 | 5.861 | 51.852 | −40.483 | 1.00 | 25.14 | C |
| ATOM | 980 | CE2 | PHE | | 139 | 7.786 | 53.096 | −41.211 | 1.00 | 25.07 | C |
| ATOM | 981 | CZ | PHE | | 139 | 6.446 | 52.768 | −41.353 | 1.00 | 24.16 | C |
| ATOM | 982 | C | PHE | | 139 | 10.661 | 49.279 | −37.942 | 1.00 | 25.70 | C |
| ATOM | 983 | O | PHE | | 139 | 11.638 | 50.027 | −38.052 | 1.00 | 26.40 | O |
| ATOM | 984 | N | ALA | | 140 | 10.653 | 48.209 | −37.155 | 1.00 | 24.62 | N |
| ATOM | 985 | CA | ALA | | 140 | 11.820 | 47.841 | −36.363 | 1.00 | 24.11 | C |
| ATOM | 986 | CB | ALA | | 140 | 11.377 | 47.346 | −34.993 | 1.00 | 22.14 | C |
| ATOM | 987 | C | ALA | | 140 | 12.617 | 46.758 | −37.096 | 1.00 | 24.35 | C |
| ATOM | 988 | O | ALA | | 140 | 13.802 | 46.543 | −36.824 | 1.00 | 23.36 | O |
| ATOM | 989 | N | LEU | | 141 | 11.958 | 46.077 | −38.028 | 1.00 | 25.20 | N |
| ATOM | 990 | CA | LEU | | 141 | 12.615 | 45.040 | −38.815 | 1.00 | 25.89 | C |
| ATOM | 991 | CB | LEU | | 141 | 11.590 | 44.234 | −39.625 | 1.00 | 25.61 | C |
| ATOM | 992 | CG | LEU | | 141 | 10.696 | 43.197 | −38.942 | 1.00 | 25.20 | C |
| ATOM | 993 | CD1 | LEU | | 141 | 9.626 | 42.727 | −39.923 | 1.00 | 22.19 | C |
| ATOM | 994 | CD2 | LEU | | 141 | 11.536 | 42.015 | −38.466 | 1.00 | 24.10 | C |
| ATOM | 995 | C | LEU | | 141 | 13.579 | 45.695 | −39.790 | 1.00 | 27.21 | C |
| ATOM | 996 | O | LEU | | 141 | 13.336 | 46.811 | −40.254 | 1.00 | 27.62 | O |
| ATOM | 997 | N | ARG | | 142 | 14.681 | 45.013 | −40.084 | 1.00 | 26.14 | N |
| ATOM | 998 | CA | ARG | | 142 | 15.638 | 45.516 | −41.054 | 1.00 | 26.46 | C |
| ATOM | 999 | CB | ARG | | 142 | 17.029 | 44.932 | −40.789 | 1.00 | 27.29 | C |
| ATOM | 1000 | CG | ARG | | 142 | 17.614 | 45.279 | −39.416 | 1.00 | 27.36 | C |
| ATOM | 1001 | CD | ARG | | 142 | 18.966 | 44.615 | −39.227 | 1.00 | 30.93 | C |
| ATOM | 1002 | NE | ARG | | 142 | 18.847 | 43.157 | −39.178 | 1.00 | 33.42 | N |
| ATOM | 1003 | CZ | ARG | | 142 | 18.768 | 42.448 | −38.057 | 1.00 | 32.99 | C |
| ATOM | 1004 | NH1 | ARG | | 142 | 18.805 | 43.062 | −36.882 | 1.00 | 32.06 | C |
| ATOM | 1005 | NH2 | ARG | | 142 | 18.638 | 41.127 | −38.111 | 1.00 | 31.91 | N |
| ATOM | 1006 | C | ARG | | 142 | 15.096 | 45.009 | −42.388 | 1.00 | 27.13 | C |
| ATOM | 1007 | O | ARG | | 142 | 14.310 | 44.062 | −42.414 | 1.00 | 25.87 | O |
| ATOM | 1008 | N | THR | | 143 | 15.496 | 45.626 | −43.493 | 1.00 | 27.88 | N |
| ATOM | 1009 | CA | THR | | 143 | 15.017 | 45.190 | −44.800 | 1.00 | 27.91 | C |
| ATOM | 1010 | CB | THR | | 143 | 15.606 | 46.054 | −45.926 | 1.00 | 30.84 | C |
| ATOM | 1011 | OG1 | THR | | 143 | 15.344 | 47.432 | −45.647 | 1.00 | 34.68 | O |
| ATOM | 1012 | CG2 | THR | | 143 | 14.976 | 45.691 | −47.266 | 1.00 | 30.42 | C |
| ATOM | 1013 | C | THR | | 143 | 15.413 | 43.737 | −45.039 | 1.00 | 27.33 | C |
| ATOM | 1014 | O | THR | | 143 | 16.585 | 43.370 | −44.895 | 1.00 | 24.82 | O |
| ATOM | 1015 | N | GLY | | 144 | 14.428 | 42.916 | −45.394 | 1.00 | 24.57 | N |
| ATOM | 1016 | CA | GLY | | 144 | 14.681 | 41.505 | −45.636 | 1.00 | 25.36 | C |
| ATOM | 1017 | C | GLY | | 144 | 14.530 | 40.618 | −44.406 | 1.00 | 24.34 | C |
| ATOM | 1018 | O | GLY | | 144 | 14.527 | 39.386 | −44.518 | 1.00 | 24.89 | O |
| ATOM | 1019 | N | GLU | | 145 | 14.399 | 41.235 | −43.234 | 1.00 | 22.32 | N |
| ATOM | 1020 | CA | GLU | | 145 | 14.257 | 40.498 | −41.976 | 1.00 | 21.64 | C |
| ATOM | 1021 | CB | GLU | | 145 | 14.792 | 41.341 | −40.810 | 1.00 | 20.75 | C |
| ATOM | 1022 | CG | GLU | | 145 | 14.707 | 40.651 | −39.452 | 1.00 | 24.18 | C |
| ATOM | 1023 | CD | GLU | | 145 | 15.227 | 41.512 | −38.314 | 1.00 | 26.80 | C |
| ATOM | 1024 | OE1 | GLU | | 145 | 15.339 | 42.746 | −38.498 | 1.00 | 26.95 | O |
| ATOM | 1025 | OE2 | GLU | | 145 | 15.509 | 40.958 | −37.229 | 1.00 | 25.61 | O |
| ATOM | 1026 | C | GLU | | 145 | 12.820 | 40.056 | −41.678 | 1.00 | 20.47 | C |
| ATOM | 1027 | O | GLU | | 145 | 11.848 | 40.691 | −42.098 | 1.00 | 20.76 | O |
| ATOM | 1028 | N | MET | | 146 | 12.699 | 38.957 | −40.943 | 1.00 | 19.00 | N |
| ATOM | 1029 | CA | MET | | 146 | 11.397 | 38.407 | −40.580 | 1.00 | 19.12 | C |
| ATOM | 1030 | CB | MET | | 146 | 11.311 | 36.940 | −41.020 | 1.00 | 20.03 | C |
| ATOM | 1031 | CG | MET | | 146 | 9.944 | 36.296 | −40.835 | 1.00 | 18.34 | C |
| ATOM | 1032 | SD | MET | | 146 | 9.907 | 34.578 | −41.401 | 1.00 | 18.12 | S |
| ATOM | 1033 | CE | MET | | 146 | 10.702 | 33.776 | −40.020 | 1.00 | 17.76 | C |
| ATOM | 1034 | C | MET | | 146 | 11.196 | 38.520 | −39.072 | 1.00 | 18.91 | C |
| ATOM | 1035 | O | MET | | 146 | 12.146 | 38.407 | −38.289 | 1.00 | 15.75 | O |
| ATOM | 1036 | N | SER | | 147 | 9.955 | 38.738 | −38.661 | 1.00 | 17.79 | N |
| ATOM | 1037 | CA | SER | | 147 | 9.651 | 38.873 | −37.247 | 1.00 | 17.17 | C |
| ATOM | 1038 | CB | SER | | 147 | 8.412 | 39.752 | −37.060 | 1.00 | 16.31 | C |
| ATOM | 1039 | OG | SER | | 147 | 7.258 | 39.013 | −37.452 | 1.00 | 16.54 | O |
| ATOM | 1040 | C | SER | | 147 | 9.351 | 37.524 | −36.617 | 1.00 | 16.94 | C |
| ATOM | 1041 | O | SER | | 147 | 9.291 | 36.498 | −37.296 | 1.00 | 15.50 | O |
| ATOM | 1042 | N | GLY | | 148 | 9.184 | 37.552 | −35.299 | 1.00 | 18.15 | N |
| ATOM | 1043 | CA | GLY | | 148 | 8.802 | 36.365 | −34.566 | 1.00 | 20.27 | C |
| ATOM | 1044 | C | GLY | | 148 | 7.299 | 36.540 | −34.455 | 1.00 | 20.89 | C |
| ATOM | 1045 | O | GLY | | 148 | 6.734 | 37.338 | −35.202 | 1.00 | 20.31 | O |
| ATOM | 1046 | N | PRO | | 149 | 6.616 | 35.830 | −33.552 | 1.00 | 22.08 | N |
| ATOM | 1047 | CD | PRO | | 149 | 7.077 | 34.695 | −32.736 | 1.00 | 21.15 | C |
| ATOM | 1048 | CA | PRO | | 149 | 5.163 | 36.003 | −33.446 | 1.00 | 21.27 | C |
| ATOM | 1049 | CB | PRO | | 149 | 4.760 | 34.946 | −32.416 | 1.00 | 22.62 | C |
| ATOM | 1050 | CG | PRO | | 149 | 5.807 | 33.894 | −32.568 | 1.00 | 23.25 | C |
| ATOM | 1051 | C | PRO | | 149 | 4.794 | 37.413 | −32.979 | 1.00 | 21.78 | C |
| ATOM | 1052 | O | PRO | | 149 | 5.242 | 37.852 | −31.920 | 1.00 | 19.81 | O |
| ATOM | 1053 | N | VAL | | 150 | 3.984 | 38.116 | −33.771 | 1.00 | 19.75 | N |
| ATOM | 1054 | CA | VAL | | 150 | 3.545 | 39.467 | −33.410 | 1.00 | 19.92 | C |
| ATOM | 1055 | CB | VAL | | 150 | 3.870 | 40.485 | −34.515 | 1.00 | 19.06 | C |
| ATOM | 1056 | CG1 | VAL | | 150 | 3.323 | 41.859 | −34.137 | 1.00 | 17.37 | C |
| ATOM | 1057 | CG2 | VAL | | 150 | 5.389 | 40.563 | −34.725 | 1.00 | 20.06 | C |
| ATOM | 1058 | C | VAL | | 150 | 2.033 | 39.439 | −33.170 | 1.00 | 20.76 | C |
| ATOM | 1059 | O | VAL | | 150 | 1.268 | 39.011 | −34.037 | 1.00 | 20.27 | O |
| ATOM | 1060 | N | PHE | | 151 | 1.605 | 39.900 | −31.996 | 1.00 | 20.33 | N |
| ATOM | 1061 | CA | PHE | | 151 | 0.185 | 39.880 | −31.651 | 1.00 | 22.41 | C |
| ATOM | 1062 | CB | PHE | | 151 | 0.009 | 39.471 | −30.176 | 1.00 | 22.53 | C |
| ATOM | 1063 | CG | PHE | | 151 | 0.625 | 38.140 | −29.831 | 1.00 | 23.14 | C |
| ATOM | 1064 | CD1 | PHE | | 151 | 2.000 | 38.012 | −29.678 | 1.00 | 21.63 | C |
| ATOM | 1065 | CD2 | PHE | | 151 | −0.172 | 37.012 | −29.668 | 1.00 | 22.87 | C |
| ATOM | 1066 | CE1 | PHE | | 151 | 2.575 | 36.776 | −29.365 | 1.00 | 23.30 | C |
| ATOM | 1067 | CE2 | PHE | | 151 | 0.393 | 35.771 | −29.354 | 1.00 | 22.95 | C |
| ATOM | 1068 | CZ | PHE | | 151 | 1.769 | 35.655 | −29.203 | 1.00 | 21.34 | C |
| ATOM | 1069 | C | PHE | | 151 | −0.555 | 41.202 | −31.893 | 1.00 | 23.09 | C |
| ATOM | 1070 | O | PHE | | 151 | −0.017 | 42.285 | −31.652 | 1.00 | 24.09 | O |
| ATOM | 1071 | N | THR | | 152 | −1.786 | 41.097 | −32.390 | 1.00 | 23.44 | N |
| ATOM | 1072 | CA | THR | | 152 | −2.648 | 42.259 | −32.634 | 1.00 | 24.04 | C |
| ATOM | 1073 | CB | THR | | 152 | −2.596 | 42.755 | −34.091 | 1.00 | 23.18 | C |
| ATOM | 1074 | OG1 | THR | | 152 | −3.228 | 41.781 | −34.935 | 1.00 | 23.10 | O |
| ATOM | 1075 | CG2 | THR | | 152 | −1.159 | 42.995 | −34.537 | 1.00 | 23.92 | C |
| ATOM | 1076 | C | THR | | 152 | −4.072 | 41.778 | −32.398 | 1.00 | 23.73 | C |
| ATOM | 1077 | O | THR | | 152 | −4.289 | 40.611 | −32.092 | 1.00 | 23.51 | O |
| ATOM | 1078 | N | ASP | | 153 | −5.048 | 42.665 | −32.561 | 1.00 | 27.44 | N |
| ATOM | 1079 | CA | ASP | | 153 | −6.437 | 42.268 | −32.370 | 1.00 | 29.62 | C |
| ATOM | 1080 | CB | ASP | | 153 | −7.341 | 43.503 | −32.292 | 1.00 | 33.87 | C |
| ATOM | 1081 | CG | ASP | | 153 | −7.091 | 44.324 | −31.045 | 1.00 | 37.22 | C |
| ATOM | 1082 | OD1 | ASP | | 153 | −7.003 | 43.724 | −29.956 | 1.00 | 38.69 | O |
| ATOM | 1083 | OD2 | ASP | | 153 | −6.988 | 45.565 | −31.147 | 1.00 | 40.78 | O |
| ATOM | 1084 | C | ASP | | 153 | −6.929 | 41.335 | −33.473 | 1.00 | 29.34 | C |
| ATOM | 1085 | O | ASP | | 153 | −7.957 | 40.682 | −33.316 | 1.00 | 30.86 | O |
| ATOM | 1086 | N | SER | | 154 | −6.200 | 41.276 | −34.588 | 1.00 | 27.23 | N |
| ATOM | 1087 | CA | SER | | 154 | −6.575 | 40.406 | −35.702 | 1.00 | 25.69 | C |
| ATOM | 1088 | CB | SER | | 154 | −5.897 | 40.854 | −37.005 | 1.00 | 26.20 | C |
| ATOM | 1089 | OG | SER | | 154 | −6.144 | 42.210 | −37.294 | 1.00 | 28.56 | O |
| ATOM | 1090 | C | SER | | 154 | −6.171 | 38.960 | −35.437 | 1.00 | 24.84 | C |
| ATOM | 1091 | O | SER | | 154 | −6.858 | 38.028 | −35.854 | 1.00 | 22.64 | O |
| ATOM | 1092 | N | GLY | | 155 | −5.043 | 38.784 | −34.750 | 1.00 | 23.43 | N |
| ATOM | 1093 | CA | GLY | | 155 | −4.549 | 37.449 | −34.462 | 1.00 | 22.29 | C |
| ATOM | 1094 | C | GLY | | 155 | −3.046 | 37.480 | −34.240 | 1.00 | 21.28 | C |
| ATOM | 1095 | O | GLY | | 155 | −2.523 | 38.419 | −33.639 | 1.00 | 22.08 | O |
| ATOM | 1096 | N | ILE | | 156 | −2.350 | 36.458 | −34.728 | 1.00 | 20.17 | N |
| ATOM | 1097 | CA | ILE | | 156 | −0.902 | 36.373 | −34.575 | 1.00 | 18.79 | C |
| ATOM | 1098 | CB | ILE | | 156 | −0.507 | 35.048 | −33.896 | 1.00 | 19.07 | C |
| ATOM | 1099 | CG2 | ILE | | 156 | 1.001 | 35.030 | −33.602 | 1.00 | 20.72 | C |
| ATOM | 1100 | CG1 | ILE | | 156 | −1.304 | 34.881 | −32.594 | 1.00 | 18.77 | C |
| ATOM | 1101 | CD1 | ILE | | 156 | −1.065 | 33.557 | −31.900 | 1.00 | 17.80 | C |
| ATOM | 1102 | C | ILE | | 156 | −0.289 | 36.438 | −35.967 | 1.00 | 19.38 | C |
| ATOM | 1103 | O | ILE | | 156 | −0.727 | 35.722 | −36.875 | 1.00 | 18.34 | O |
| ATOM | 1104 | N | HIS | | 157 | 0.722 | 37.286 | −36.135 | 1.00 | 18.01 | N |
| ATOM | 1105 | CA | HIS | | 157 | 1.356 | 37.451 | −37.438 | 1.00 | 19.24 | C |
| ATOM | 1106 | CB | HIS | | 157 | 1.283 | 38.910 | −37.916 | 1.00 | 19.95 | C |
| ATOM | 1107 | CG | HIS | | 157 | −0.031 | 39.587 | −37.688 | 1.00 | 21.88 | C |
| ATOM | 1108 | CD2 | HIS | | 157 | −0.720 | 39.848 | −36.551 | 1.00 | 21.84 | C |
| ATOM | 1109 | ND1 | HIS | | 157 | −0.733 | 40.190 | −38.708 | 1.00 | 20.39 | N |
| ATOM | 1110 | CE1 | HIS | | 157 | −1.795 | 40.799 | −38.211 | 1.00 | 23.64 | C |
| ATOM | 1111 | NE2 | HIS | | 157 | −1.811 | 40.607 | −36.904 | 1.00 | 21.95 | N |
| ATOM | 1112 | C | HIS | | 157 | 2.839 | 37.102 | −37.484 | 1.00 | 19.21 | C |
| ATOM | 1113 | O | HIS | | 157 | 3.541 | 37.156 | −36.475 | 1.00 | 19.34 | O |
| ATOM | 1114 | N | ILE | | 158 | 3.288 | 36.751 | −38.686 | 1.00 | 18.54 | N |
| ATOM | 1115 | CA | ILE | | 158 | 4.688 | 36.511 | −38.975 | 1.00 | 17.73 | C |
| ATOM | 1116 | CB | ILE | | 158 | 4.960 | 35.163 | −39.644 | 1.00 | 19.32 | C |
| ATOM | 1117 | CG2 | ILE | | 158 | 6.413 | 35.120 | −40.090 | 1.00 | 20.23 | C |
| ATOM | 1118 | CG1 | ILE | | 158 | 4.689 | 34.016 | −38.668 | 1.00 | 19.48 | C |
| ATOM | 1119 | CD1 | ILE | | 158 | 4.783 | 32.633 | −39.314 | 1.00 | 20.27 | C |
| ATOM | 1120 | C | ILE | | 158 | 4.852 | 37.615 | −40.016 | 1.00 | 18.12 | C |
| ATOM | 1121 | O | ILE | | 158 | 4.118 | 37.649 | −41.013 | 1.00 | 18.44 | O |
| ATOM | 1122 | N | ILE | | 159 | 5.800 | 38.513 | −39.784 | 1.00 | 17.05 | N |
| ATOM | 1123 | CA | ILE | | 159 | 6.003 | 39.651 | −40.670 | 1.00 | 18.79 | C |
| ATOM | 1124 | CB | ILE | | 159 | 5.913 | 40.975 | −39.870 | 1.00 | 18.39 | C |
| ATOM | 1125 | CG2 | ILE | | 159 | 6.015 | 42.170 | −40.815 | 1.00 | 19.94 | C |
| ATOM | 1126 | CG1 | ILE | | 159 | 4.610 | 41.001 | −39.062 | 1.00 | 20.68 | C |
| ATOM | 1127 | CD1 | ILE | | 159 | 4.461 | 42.216 | −38.129 | 1.00 | 17.02 | C |
| ATOM | 1128 | C | ILE | | 159 | 7.328 | 39.642 | −41.417 | 1.00 | 18.45 | C |
| ATOM | 1129 | O | ILE | | 159 | 8.383 | 39.418 | −40.831 | 1.00 | 20.19 | O |
| ATOM | 1130 | N | LEU | | 160 | 7.263 | 39.886 | −42.720 | 1.00 | 18.39 | N |
| ATOM | 1131 | CA | LEU | | 160 | 8.476 | 39.947 | −43.528 | 1.00 | 18.91 | C |
| ATOM | 1132 | CB | LEU | | 160 | 8.453 | 38.901 | −44.650 | 1.00 | 16.90 | C |
| ATOM | 1133 | CG | LEU | | 160 | 9.663 | 38.977 | −45.592 | 1.00 | 18.75 | C |
| ATOM | 1134 | CD1 | LEU | | 160 | 10.915 | 38.508 | −44.852 | 1.00 | 19.35 | C |
| ATOM | 1135 | CD2 | LEU | | 160 | 9.436 | 38.117 | −46.829 | 1.00 | 17.69 | C |
| ATOM | 1136 | C | LEU | | 160 | 8.566 | 41.333 | −44.140 | 1.00 | 19.09 | C |
| ATOM | 1137 | O | LEU | | 160 | 7.689 | 41.736 | −44.901 | 1.00 | 20.34 | O |
| ATOM | 1138 | N | ARG | | 161 | 9.616 | 42.068 | −43.792 | 1.00 | 21.02 | N |
| ATOM | 1139 | CA | ARG | | 161 | 9.808 | 43.397 | −44.346 | 1.00 | 22.02 | C |
| ATOM | 1140 | CB | ARG | | 161 | 10.633 | 44.282 | −43.412 | 1.00 | 21.72 | C |
| ATOM | 1141 | CG | ARG | | 161 | 10.864 | 45.646 | −44.032 | 1.00 | 24.85 | C |
| ATOM | 1142 | CD | ARG | | 161 | 11.712 | 46.578 | −43.197 | 1.00 | 27.39 | C |
| ATOM | 1143 | NE | ARG | | 161 | 12.036 | 47.761 | −43.992 | 1.00 | 27.33 | N |
| ATOM | 1144 | CZ | ARG | | 161 | 12.907 | 48.695 | −43.634 | 1.00 | 29.68 | C |
| ATOM | 1145 | NH1 | ARG | | 161 | 13.551 | 48.596 | −42.478 | 1.00 | 28.60 | N |
| ATOM | 1146 | NH2 | ARG | | 161 | 13.144 | 49.719 | −44.443 | 1.00 | 27.90 | N |
| ATOM | 1147 | C | ARG | | 161 | 10.538 | 43.236 | −45.673 | 1.00 | 21.63 | C |
| ATOM | 1148 | O | ARG | | 161 | 11.654 | 42.729 | −45.714 | 1.00 | 21.71 | O |
| ATOM | 1149 | N | THR | | 162 | 9.905 | 43.676 | −46.751 | 1.00 | 20.78 | N |
| ATOM | 1150 | CA | THR | | 162 | 10.487 | 43.541 | −48.077 | 1.00 | 22.60 | C |
| ATOM | 1151 | CB | THR | | 162 | 9.421 | 43.066 | −49.085 | 1.00 | 22.27 | C |
| ATOM | 1152 | OG1 | THR | | 162 | 8.274 | 43.921 | −49.013 | 1.00 | 22.96 | O |
| ATOM | 1153 | CG2 | THR | | 162 | 8.992 | 41.636 | −48.764 | 1.00 | 22.61 | C |
| ATOM | 1154 | C | THR | | 162 | 11.146 | 44.814 | −48.591 | 1.00 | 23.00 | C |
| ATOM | 1155 | O | THR | | 162 | 12.029 | 44.753 | −49.441 | 1.00 | 22.65 | O |
| ATOM | 1156 | N | GLU | | 163 | 10.718 | 45.961 | −48.078 | 1.00 | 24.32 | N |
| ATOM | 1157 | CA | GLU | | 163 | 11.284 | 47.242 | −48.490 | 1.00 | 26.46 | C |
| ATOM | 1158 | CB | GLU | | 163 | 10.445 | 47.850 | −49.620 | 1.00 | 28.32 | C |
| ATOM | 1159 | CG | GLU | | 163 | 10.507 | 47.101 | −50.943 | 1.00 | 30.56 | C |
| ATOM | 1160 | CD | GLU | | 163 | 9.525 | 47.652 | −51.969 | 1.00 | 32.59 | C |
| ATOM | 1161 | OE1 | GLU | | 163 | 9.172 | 48.850 | −51.881 | 1.00 | 33.11 | O |
| ATOM | 1162 | OE2 | GLU | | 163 | 9.115 | 46.891 | −52.870 | 1.00 | 33.50 | O |
| ATOM | 1163 | C | GLU | | 163 | 11.304 | 48.214 | −47.311 | 1.00 | 27.33 | C |
| ATOM | 1164 | O | GLU | | 163 | 10.445 | 48.066 | −46.416 | 1.00 | 28.13 | O |
| ATOM | 1165 | OXT | GLU | | 163 | 12.156 | 49.128 | −47.312 | 1.00 | 27.53 | O |
| ATOM | 1166 | OH2 | WAT | S | 1 | 7.893 | 41.331 | −27.148 | 1.00 | 20.05 | O |
| ATOM | 1167 | OH2 | WAT | S | 2 | 17.346 | 30.491 | −41.153 | 1.00 | 19.42 | O |
| ATOM | 1168 | OH2 | WAT | S | 3 | −4.284 | 32.127 | −51.291 | 1.00 | 20.44 | O |
| ATOM | 1169 | OH2 | WAT | S | 4 | −1.352 | 36.952 | −45.720 | 1.00 | 20.58 | O |
| ATOM | 1170 | OH2 | WAT | S | 5 | 2.568 | 25.068 | −38.010 | 1.00 | 22.22 | O |
| ATOM | 1171 | OH2 | WAT | S | 6 | 15.546 | 44.418 | −36.331 | 1.00 | 22.22 | O |
| ATOM | 1172 | OH2 | WAT | S | 7 | 6.032 | 42.545 | −49.458 | 1.00 | 21.44 | O |
| ATOM | 1173 | OH2 | WAT | S | 8 | 13.801 | 42.245 | −35.291 | 1.00 | 26.16 | O |
| ATOM | 1174 | OH2 | WAT | S | 9 | −0.917 | 36.386 | −56.614 | 1.00 | 22.75 | O |
| ATOM | 1175 | OH2 | WAT | S | 10 | 9.461 | 29.022 | −34.080 | 1.00 | 23.06 | O |
| ATOM | 1176 | OH2 | WAT | S | 11 | −0.374 | 33.895 | −53.631 | 1.00 | 22.72 | O |
| ATOM | 1177 | OH2 | WAT | S | 12 | 1.353 | 54.881 | −34.353 | 1.00 | 29.58 | O |
| ATOM | 1178 | OH2 | WAT | S | 13 | −10.315 | 27.273 | −36.239 | 1.00 | 27.61 | O |
| ATOM | 1179 | OH2 | WAT | S | 14 | −3.068 | 27.728 | −39.878 | 1.00 | 22.17 | O |
| ATOM | 1180 | OH2 | WAT | S | 15 | 20.327 | 33.128 | −36.148 | 1.00 | 23.99 | O |
| ATOM | 1181 | OH2 | WAT | S | 16 | 14.045 | 43.523 | −21.728 | 1.00 | 29.71 | O |
| ATOM | 1182 | OH2 | WAT | S | 17 | −3.857 | 45.355 | −32.255 | 1.00 | 29.16 | O |
| ATOM | 1183 | OH2 | WAT | S | 18 | 1.002 | 51.896 | −39.707 | 1.00 | 38.48 | O |
| ATOM | 1184 | OH2 | WAT | S | 19 | 12.908 | 30.336 | −46.148 | 1.00 | 22.55 | O |
| ATOM | 1185 | OH2 | WAT | S | 20 | 16.527 | 38.495 | −36.902 | 1.00 | 27.58 | O |
| ATOM | 1186 | OH2 | WAT | S | 21 | −2.331 | 24.038 | −48.848 | 1.00 | 32.35 | O |
| ATOM | 1187 | OH2 | WAT | S | 22 | 0.917 | 34.371 | −56.164 | 1.00 | 27.90 | O |
| ATOM | 1188 | OH2 | WAT | S | 23 | 4.931 | 38.281 | −50.188 | 1.00 | 28.56 | O |
| ATOM | 1189 | OH2 | WAT | S | 24 | 13.874 | 36.942 | −43.530 | 1.00 | 26.39 | O |
| ATOM | 1190 | OH2 | WAT | S | 25 | 17.618 | 32.933 | −36.603 | 1.00 | 30.83 | O |
| ATOM | 1191 | OH2 | WAT | S | 26 | −8.129 | 29.096 | −40.568 | 1.00 | 27.75 | O |
| ATOM | 1192 | OH2 | WAT | S | 27 | −3.679 | 37.727 | −30.843 | 1.00 | 31.15 | O |
| ATOM | 1193 | OH2 | WAT | S | 28 | 9.282 | 50.419 | −34.337 | 1.00 | 30.75 | O |
| ATOM | 1194 | OH2 | WAT | S | 29 | 27.012 | 23.613 | −24.655 | 1.00 | 29.62 | O |
| ATOM | 1195 | OH2 | WAT | S | 30 | −4.557 | 29.710 | −40.751 | 1.00 | 28.97 | O |
| ATOM | 1196 | OH2 | WAT | S | 31 | −0.997 | 24.226 | −41.494 | 1.00 | 33.22 | O |
| ATOM | 1197 | OH2 | WAT | S | 32 | 16.353 | 29.577 | −24.662 | 1.00 | 31.58 | O |
| ATOM | 1198 | OH2 | WAT | S | 33 | 25.743 | 33.873 | −30.490 | 1.00 | 25.85 | O |
| ATOM | 1199 | OH2 | WAT | S | 34 | 5.332 | 45.143 | −31.658 | 1.00 | 30.16 | O |
| ATOM | 1200 | OH2 | WAT | S | 35 | 14.920 | 35.643 | −24.808 | 1.00 | 33.85 | O |
| ATOM | 1201 | OH2 | WAT | S | 36 | 15.290 | 31.392 | −31.500 | 1.00 | 27.63 | O |
| ATOM | 1202 | OH2 | WAT | S | 37 | −6.894 | 26.860 | −47.045 | 1.00 | 33.52 | O |
| ATOM | 1203 | OH2 | WAT | S | 38 | −7.398 | 46.846 | −28.941 | 1.00 | 51.90 | O |
| ATOM | 1204 | OH2 | WAT | S | 39 | 10.653 | 22.662 | −39.314 | 1.00 | 45.55 | O |
| ATOM | 1205 | OH2 | WAT | S | 40 | 18.244 | 24.600 | −19.261 | 1.00 | 38.82 | O |
| ATOM | 1206 | OH2 | WAT | S | 41 | −15.687 | 27.400 | −43.454 | 1.00 | 34.30 | O |
| ATOM | 1207 | OH2 | WAT | S | 42 | −18.597 | 27.618 | −44.427 | 1.00 | 32.89 | O |
| ATOM | 1208 | OH2 | WAT | S | 43 | 6.049 | 37.358 | −53.977 | 1.00 | 36.68 | O |
| ATOM | 1209 | OH2 | WAT | S | 44 | 19.052 | 43.889 | −43.831 | 1.00 | 34.72 | O |
| ATOM | 1210 | OH2 | WAT | S | 45 | 15.461 | 47.913 | −34.992 | 1.00 | 31.23 | O |
| ATOM | 1211 | OH2 | WAT | S | 46 | 20.837 | 37.225 | −38.207 | 1.00 | 28.28 | O |
| ATOM | 1212 | OH2 | WAT | S | 47 | 19.507 | 45.526 | −23.866 | 1.00 | 47.05 | O |
| ATOM | 1213 | OH2 | WAT | S | 48 | 17.325 | 48.028 | −43.296 | 1.00 | 48.61 | O |
| ATOM | 1214 | OH2 | WAT | S | 49 | 17.907 | 34.411 | −21.643 | 1.00 | 38.49 | O |
| ATOM | 1215 | OH2 | WAT | S | 50 | 5.070 | 47.615 | −29.795 | 1.00 | 38.43 | O |
| ATOM | 1216 | OH2 | WAT | S | 51 | −8.466 | 32.182 | −28.123 | 1.00 | 37.74 | O |
| ATOM | 1217 | OH2 | WAT | S | 52 | 11.537 | 34.750 | −30.892 | 1.00 | 38.98 | O |
| ATOM | 1218 | O1 | PGA | B | 180 | 16.565 | 30.648 | −29.156 | 1.00 | 34.95 | O |
| ATOM | 1219 | C2 | PGA | B | 180 | 17.916 | 30.499 | −29.151 | 1.00 | 30.97 | C |
| ATOM | 1220 | C3 | PGA | B | 180 | 18.549 | 31.257 | −30.326 | 1.00 | 29.19 | C |
| ATOM | 1221 | O4 | PGA | B | 180 | 18.179 | 30.717 | −31.480 | 1.00 | 27.98 | O |
| ATOM | 1222 | C5 | PGA | B | 180 | 18.543 | 31.454 | −32.527 | 1.00 | 24.65 | C |
| ATOM | 1223 | C6 | PGA | B | 180 | 18.061 | 30.765 | −33.815 | 1.00 | 27.19 | C |
| ATOM | 1224 | O7 | PGA | B | 180 | 16.757 | 30.428 | −33.686 | 1.00 | 28.41 | O |
| ATOM | 1225 | C8 | PGA | B | 180 | 16.320 | 29.679 | −34.708 | 1.00 | 26.90 | C |
| ATOM | 1226 | C9 | PGA | B | 180 | 14.835 | 29.337 | −34.488 | 1.00 | 27.50 | C |
| ATOM | 1227 | O10 | PGA | B | 180 | 14.108 | 30.481 | −34.359 | 1.00 | 28.64 | O |
| ATOM | 1228 | C11 | PGA | B | 180 | 12.816 | 30.227 | −34.105 | 1.00 | 27.73 | C |
| ATOM | 1229 | C12 | PGA | B | 180 | 12.129 | 31.509 | −33.596 | 1.00 | 29.25 | C |
| ATOM | 1230 | O13 | PGA | B | 180 | 12.280 | 32.504 | −34.479 | 1.00 | 27.90 | O |
| ATOM | 1231 | C14 | PGA | B | 180 | 11.635 | 33.590 | −34.048 | 1.00 | 30.91 | C |
| ATOM | 1232 | C15 | PGA | B | 180 | 11.739 | 34.725 | −35.087 | 1.00 | 30.86 | C |
| ATOM | 1233 | O16 | PGA | B | 180 | 13.005 | 35.098 | −35.301 | 1.00 | 28.94 | O |
| ATOM | 1234 | C17 | PGA | B | 180 | 13.004 | 35.725 | −36.434 | 1.00 | 35.98 | C |
| ATOM | 1235 | C18 | PGA | B | 180 | 14.387 | 36.187 | −36.862 | 1.00 | 36.33 | C |
| ATOM | 1236 | O19 | PGA | B | 180 | 14.291 | 36.621 | −38.093 | 1.00 | 34.83 | O |
| ATOM | 1237 | O1 | PGB | C | 181 | −2.961 | 45.735 | −38.887 | 1.00 | 35.85 | O |
| ATOM | 1238 | C2 | PGB | C | 181 | −2.391 | 44.535 | −38.920 | 1.00 | 38.14 | C |
| ATOM | 1239 | C3 | PGB | C | 181 | −2.092 | 44.102 | −40.376 | 1.00 | 37.17 | C |
| ATOM | 1240 | O4 | PGB | C | 181 | −3.218 | 43.989 | −41.089 | 1.00 | 39.98 | O |
| ATOM | 1241 | C5 | PGB | C | 181 | −2.951 | 43.671 | −42.381 | 1.00 | 41.36 | C |
| ATOM | 1242 | C6 | PGB | C | 181 | −4.245 | 43.666 | −43.231 | 1.00 | 43.82 | C |
| ATOM | 1243 | O7 | PGB | C | 181 | −4.709 | 44.927 | −43.388 | 1.00 | 45.09 | O |
| ATOM | 1244 | S | SO4 | | 190 | −9.615 | 41.396 | −38.442 | 1.00 | 58.70 | S |
| ATOM | 1245 | O1 | SO4 | | 190 | −9.696 | 40.211 | −37.557 | 1.00 | 58.17 | O |
| ATOM | 1246 | O2 | SO4 | | 190 | −8.575 | 42.323 | −37.939 | 1.00 | 57.55 | O |
| ATOM | 1247 | O3 | SO4 | | 190 | −10.923 | 42.101 | −38.448 | 1.00 | 58.85 | O |
| ATOM | 1248 | O4 | SO4 | | 190 | −9.294 | 40.994 | −39.836 | 1.00 | 57.04 | O |
| END | | | | | | | | | | | |

### Example 2

### Activity-Based Assay for Inhibitor Evaluation

The proline isomerase activity assay is based on the method described by Fisher et al. (Biomed. Biochim. Acta, 1984, 43:1101-1111). Specifically, the enzyme (112 ng) was preincubated with 72 mM substrate at 4° C. for 30 minutes in an 80 μL reaction volume containing 0.02 mg/μL BSA, 0.8 mM DTT, and 35 mM HEPES (pH 7.8). Proteolysis of the substrate was initiated by the addition of 80 μL of trypsin at 0.4 mg/mL in 35 mM HEPES (pH 7.8) and the release of p-nitroaniline was monitored every 10 seconds at 390 nm using a microplate reader (MRD/8V/DIAS, Dynex Technologies). Inhibition studies were preformed by adding 5 μL of inhibitors added in the pre-incubation mix. Inhibitors were at 0.4 mg/mL in 10% DMSO.

### Example 3

### Structural Determination of Pin1 R14A/Inhibitor Complexes

Pin1 R14A/Inhibitor Complex

Crystals of Pin1 R14A were obtained as previously specified in Example 1. Crystals of Pin1 R14A were transferred to a solution of 50 mM HEPES (pH 7.5), 30% PEG containing the various inhibitors.

The Pin1 R14A crystal form yields stable crystals that diffract to high resolution. The proline isomerase (peptidyl-prolyl cis-trans isomerase) active site is exposed within the crystal lattice to the large solvent channels within the crystal. The immediate area around the center of this active site is not involved with any critical protein-protein crystal contacts. Thus, soluble compounds have access to the PPIase active site of already formed crystals. This then allows for the soaking of Pin1 R14A crystals in solutions containing any desired soluble ligand, giving the ligand the access needed to bind to the active site. Crystal soaking does not then require a reoptimization of the crystallization conditions, and any number of solutions can be screened, thus increasing the speed at which ligand-protein structures may be determined. Pin1 R14A/Pin-077 Interactions The crystal structure of the co-complex with PIN-077 (the coordinates set is set forth in Table 2), shows that the sulfate moiety of this molecule makes four hydrogen bond contacts in the basic pocket (with Ser154, Arg69, and Lys63) and that the hydroxyl groups on the proximal ring hydrogen bond to the nitrogen of the Arg69 sidechain and to Lys63. This interaction with sulfate moiety of PIN-077 is different than that observed for the sulfate ion in the Pin1 R14A crystal structure, in that PIN-077 binds deeper into the basic pocket and displaces a water molecule that was interacting with Arg69 peptide nitrogen. Investigation of the remainder of the structure demonstrated that the remaining two rings of PIN-077 do not bind in the active site cavity and protrude out of the active site. Interestingly, it was observed that a polyethylene glycol molecule was bound in the hydrophobic pocket showing the formation of a hydrogen bond between the hydroxyl group of the polyethylene glycol molecule and a backbone nitrogen atom (Gln131) of the Pin1 polypeptide.

TABLE 2
Set of atomic coordinates of Pin1 R14A with PIN-077 and PEG bound in the active site.

| | | | | | | | | | | | |
|---|---|---|---|---|---|---|---|---|---|---|---|
| ATOM | 1 | CB | LYS | | 6 | 4.089 | 106.181 | −14.909 | 1.00 | 62.82 | C |
| ATOM | 2 | CG | LYS | | 6 | 4.748 | 105.929 | −13.561 | 1.00 | 63.71 | C |
| ATOM | 3 | CD | LYS | | 6 | 4.011 | 106.671 | −12.454 | 1.00 | 64.79 | C |
| ATOM | 4 | CE | LYS | | 6 | 4.710 | 106.508 | −11.111 | 1.00 | 65.58 | C |
| ATOM | 5 | NZ | LYS | | 6 | 4.028 | 107.272 | −10.024 | 1.00 | 65.16 | N |
| ATOM | 6 | C | LYS | | 6 | 4.690 | 104.085 | −16.160 | 1.00 | 60.82 | C |
| ATOM | 7 | O | LYS | | 6 | 4.132 | 103.469 | −15.247 | 1.00 | 60.67 | O |
| ATOM | 8 | N | LYS | | 6 | 4.340 | 106.209 | −17.370 | 1.00 | 62.35 | N |
| ATOM | 9 | CA | LYS | | 6 | 4.851 | 105.605 | −16.107 | 1.00 | 61.77 | C |
| ATOM | 10 | N | LEU | | 7 | 5.180 | 103.499 | −17.250 | 1.00 | 58.91 | N |
| ATOM | 11 | CA | LEU | | 7 | 5.132 | 102.056 | −17.478 | 1.00 | 56.07 | C |
| ATOM | 12 | CB | LEU | | 7 | 5.498 | 101.295 | −16.197 | 1.00 | 56.64 | C |
| ATOM | 13 | CG | LEU | | 7 | 6.919 | 101.478 | −15.652 | 1.00 | 56.81 | C |
| ATOM | 14 | CD1 | LEU | | 7 | 7.099 | 100.654 | −14.384 | 1.00 | 56.09 | C |
| ATOM | 15 | CD2 | LEU | | 7 | 7.926 | 101.057 | −16.708 | 1.00 | 55.86 | C |
| ATOM | 16 | C | LEU | | 7 | 3.792 | 101.547 | −17.998 | 1.00 | 53.87 | C |
| ATOM | 17 | O | LEU | | 7 | 2.774 | 101.634 | −17.314 | 1.00 | 52.42 | O |
| ATOM | 18 | N | PRO | | 8 | 3.783 | 101.009 | −19.228 | 1.00 | 52.27 | N |
| ATOM | 19 | CD | PRO | | 8 | 4.915 | 101.003 | −20.172 | 1.00 | 52.41 | C |
| ATOM | 20 | CA | PRO | | 8 | 2.578 | 100.468 | −19.866 | 1.00 | 51.26 | C |
| ATOM | 21 | CB | PRO | | 8 | 3.115 | 99.896 | −21.176 | 1.00 | 51.93 | C |
| ATOM | 22 | CG | PRO | | 8 | 4.222 | 100.840 | −21.509 | 1.00 | 52.37 | C |
| ATOM | 23 | C | PRO | | 8 | 1.915 | 99.400 | −18.989 | 1.00 | 49.93 | C |
| ATOM | 24 | O | PRO | | 8 | 2.487 | 98.963 | −17.988 | 1.00 | 49.55 | O |
| ATOM | 25 | N | PRO | | 9 | 0.703 | 98.957 | −19.367 | 1.00 | 48.16 | N |
| ATOM | 26 | CD | PRO | | 9 | −0.041 | 99.406 | −20.558 | 1.00 | 47.50 | C |
| ATOM | 27 | CA | PRO | | 9 | −0.066 | 97.944 | −18.632 | 1.00 | 46.31 | C |
| ATOM | 28 | CB | PRO | | 9 | −1.220 | 97.636 | −19.587 | 1.00 | 46.59 | C |
| ATOM | 29 | CG | PRO | | 9 | −1.452 | 98.954 | −20.247 | 1.00 | 47.31 | C |
| ATOM | 30 | C | PRO | | 9 | 0.671 | 96.673 | −18.194 | 1.00 | 44.67 | C |
| ATOM | 31 | O | PRO | | 9 | 1.154 | 95.898 | −19.026 | 1.00 | 43.04 | O |
| ATOM | 32 | N | GLY | | 10 | 0.744 | 96.473 | −16.877 | 1.00 | 43.13 | N |
| ATOM | 33 | CA | GLY | | 10 | 1.374 | 95.285 | −16.327 | 1.00 | 40.91 | C |
| ATOM | 34 | C | GLY | | 10 | 2.867 | 95.289 | −16.042 | 1.00 | 38.94 | C |
| ATOM | 35 | O | GLY | | 10 | 3.392 | 94.294 | −15.543 | 1.00 | 36.23 | O |
| ATOM | 36 | N | TRP | | 11 | 3.553 | 96.388 | −16.344 | 1.00 | 38.92 | N |
| ATOM | 37 | CA | TRP | | 11 | 4.995 | 96.468 | −16.113 | 1.00 | 38.40 | C |
| ATOM | 38 | CB | TRP | | 11 | 5.649 | 97.420 | −17.119 | 1.00 | 35.93 | C |
| ATOM | 39 | CG | TRP | | 11 | 5.669 | 96.894 | −18.512 | 1.00 | 32.90 | C |
| ATOM | 40 | CD2 | TRP | | 11 | 6.601 | 95.951 | −19.058 | 1.00 | 33.87 | C |
| ATOM | 41 | CE2 | TRP | | 11 | 6.221 | 95.715 | −20.399 | 1.00 | 32.37 | C |
| ATOM | 42 | CE3 | TRP | | 11 | 7.722 | 95.281 | −18.543 | 1.00 | 31.97 | C |
| ATOM | 43 | CD1 | TRP | | 11 | 4.788 | 97.183 | −19.512 | 1.00 | 32.74 | C |
| ATOM | 44 | NE1 | TRP | | 11 | 5.112 | 96.479 | −20.650 | 1.00 | 32.67 | N |
| ATOM | 45 | CZ2 | TRP | | 11 | 6.920 | 94.839 | −21.232 | 1.00 | 31.65 | C |
| ATOM | 46 | CZ3 | TRP | | 11 | 8.416 | 94.410 | −19.371 | 1.00 | 31.60 | C |
| ATOM | 47 | CH2 | TRP | | 11 | 8.013 | 94.197 | −20.704 | 1.00 | 32.70 | C |
| ATOM | 48 | C | TRP | | 11 | 5.381 | 96.897 | −14.703 | 1.00 | 39.33 | C |
| ATOM | 49 | O | TRP | | 11 | 4.680 | 97.684 | −14.065 | 1.00 | 39.85 | O |
| ATOM | 50 | N | GLU | | 12 | 6.508 | 96.365 | −14.229 | 1.00 | 39.72 | N |
| ATOM | 51 | CA | GLU | | 12 | 7.034 | 96.681 | −12.903 | 1.00 | 40.19 | C |
| ATOM | 52 | CB | GLU | | 12 | 6.582 | 95.640 | −11.876 | 1.00 | 42.16 | C |
| ATOM | 53 | CG | GLU | | 12 | 7.169 | 94.253 | −12.104 | 1.00 | 46.75 | C |
| ATOM | 54 | CD | GLU | | 12 | 6.944 | 93.312 | −10.934 | 1.00 | 48.75 | C |
| ATOM | 55 | OE1 | GLU | | 12 | 5.776 | 93.129 | −10.525 | 1.00 | 50.08 | O |
| ATOM | 56 | OE2 | GLU | | 12 | 7.938 | 92.750 | −10.428 | 1.00 | 50.44 | O |
| ATOM | 57 | C | GLU | | 12 | 8.564 | 96.717 | −12.930 | 1.00 | 40.03 | C |
| ATOM | 58 | O | GLU | | 12 | 9.202 | 96.046 | −13.744 | 1.00 | 37.52 | O |
| ATOM | 59 | N | LYS | | 13 | 9.145 | 97.502 | −12.029 | 1.00 | 39.79 | N |
| ATOM | 60 | CA | LYS | | 13 | 10.594 | 97.628 | −11.937 | 1.00 | 39.84 | C |
| ATOM | 61 | CB | LYS | | 13 | 10.964 | 98.930 | −11.221 | 1.00 | 42.55 | C |
| ATOM | 62 | CG | LYS | | 13 | 12.458 | 99.209 | −11.144 | 1.00 | 45.64 | C |
| ATOM | 63 | CD | LYS | | 13 | 12.765 | 100.361 | −10.189 | 1.00 | 47.35 | C |
| ATOM | 64 | CE | LYS | | 13 | 12.123 | 101.670 | −10.646 | 1.00 | 49.42 | C |
| ATOM | 65 | NZ | LYS | | 13 | 12.373 | 102.793 | −9.687 | 1.00 | 49.08 | N |
| ATOM | 66 | C | LYS | | 13 | 11.132 | 96.443 | −11.153 | 1.00 | 39.36 | C |
| ATOM | 67 | O | LYS | | 13 | 10.535 | 96.022 | −10.160 | 1.00 | 39.91 | O |
| ATOM | 68 | N | ALA | | 14 | 12.255 | 95.895 | −11.600 | 1.00 | 37.26 | N |
| ATOM | 69 | CA | ALA | | 14 | 12.850 | 94.761 | −10.912 | 1.00 | 36.51 | C |
| ATOM | 70 | CB | ALA | | 14 | 12.538 | 93.468 | −11.657 | 1.00 | 36.81 | C |
| ATOM | 71 | C | ALA | | 14 | 14.354 | 94.941 | −10.773 | 1.00 | 35.92 | C |
| ATOM | 72 | O | ALA | | 14 | 14.941 | 95.866 | −11.341 | 1.00 | 33.23 | O |
| ATOM | 73 | N | MET | | 15 | 14.969 | 94.039 | −10.018 | 1.00 | 36.46 | N |
| ATOM | 74 | CA | MET | | 15 | 16.401 | 94.079 | −9.770 | 1.00 | 36.73 | C |
| ATOM | 75 | CB | MET | | 15 | 16.643 | 94.355 | −8.285 | 1.00 | 40.40 | C |
| ATOM | 76 | CG | MET | | 15 | 18.099 | 94.471 | −7.906 | 1.00 | 43.53 | C |
| ATOM | 77 | SD | MET | | 15 | 18.814 | 95.858 | −8.764 | 1.00 | 48.38 | S |
| ATOM | 78 | CE | MET | | 15 | 18.202 | 97.216 | −7.757 | 1.00 | 45.86 | C |
| ATOM | 79 | C | MET | | 15 | 17.049 | 92.754 | −10.155 | 1.00 | 35.73 | C |
| ATOM | 80 | O | MET | | 15 | 16.668 | 91.704 | −9.638 | 1.00 | 34.75 | O |
| ATOM | 81 | N | SER | | 16 | 18.026 | 92.794 | −11.055 | 1.00 | 34.08 | N |
| ATOM | 82 | CA | SER | | 16 | 18.700 | 91.565 | −11.462 | 1.00 | 34.19 | C |
| ATOM | 83 | CB | SER | | 16 | 19.674 | 91.829 | −12.608 | 1.00 | 34.05 | C |
| ATOM | 84 | OG | SER | | 16 | 20.389 | 90.645 | −12.924 | 1.00 | 33.48 | O |
| ATOM | 85 | C | SER | | 16 | 19.467 | 90.969 | −10.288 | 1.00 | 34.88 | C |
| ATOM | 86 | O | SER | | 16 | 20.165 | 91.681 | −9.568 | 1.00 | 34.56 | O |
| ATOM | 87 | N | ARG | | 17 | 19.335 | 89.660 | −10.106 | 1.00 | 36.97 | N |
| ATOM | 88 | CA | ARG | | 17 | 20.008 | 88.950 | −9.022 | 1.00 | 39.64 | C |
| ATOM | 89 | CB | ARG | | 17 | 19.350 | 87.587 | −8.795 | 1.00 | 40.65 | C |
| ATOM | 90 | CG | ARG | | 17 | 17.993 | 87.651 | −8.135 | 1.00 | 41.99 | C |
| ATOM | 91 | CD | ARG | | 17 | 18.109 | 88.207 | −6.732 | 1.00 | 42.76 | C |
| ATOM | 92 | NE | ARG | | 17 | 16.799 | 88.434 | −6.135 | 1.00 | 44.85 | N |
| ATOM | 93 | CZ | ARG | | 17 | 15.966 | 87.471 | −5.753 | 1.00 | 44.86 | C |
| ATOM | 94 | NH1 | ARG | | 17 | 16.299 | 86.193 | −5.897 | 1.00 | 43.83 | N |
| ATOM | 95 | NH2 | ARG | | 17 | 14.793 | 87.793 | −5.232 | 1.00 | 45.08 | N |
| ATOM | 96 | C | ARG | | 17 | 21.486 | 88.732 | −9.301 | 1.00 | 40.72 | C |
| ATOM | 97 | O | ARG | | 17 | 22.297 | 88.653 | −8.378 | 1.00 | 40.92 | O |
| ATOM | 98 | N | SER | | 18 | 21.828 | 88.626 | −10.579 | 1.00 | 41.09 | N |
| ATOM | 99 | CA | SER | | 18 | 23.205 | 88.387 | −10.988 | 1.00 | 41.84 | C |
| ATOM | 100 | CB | SER | | 18 | 23.220 | 87.624 | −12.317 | 1.00 | 42.02 | C |
| ATOM | 101 | OG | SER | | 18 | 22.615 | 86.351 | −12.178 | 1.00 | 44.52 | O |
| ATOM | 102 | C | SER | | 18 | 24.062 | 89.641 | −11.117 | 1.00 | 41.56 | C |
| ATOM | 103 | O | SER | | 18 | 25.210 | 89.664 | −10.662 | 1.00 | 43.19 | O |
| ATOM | 104 | N | SER | | 19 | 23.506 | 90.684 | −11.725 | 1.00 | 40.24 | N |
| ATOM | 105 | CA | SER | | 19 | 24.244 | 91.921 | −11.942 | 1.00 | 38.48 | C |
| ATOM | 106 | CB | SER | | 19 | 24.111 | 92.336 | −13.409 | 1.00 | 40.39 | C |
| ATOM | 107 | OG | SER | | 19 | 22.750 | 92.373 | −13.801 | 1.00 | 39.90 | O |
| ATOM | 108 | C | SER | | 19 | 23.857 | 93.093 | −11.049 | 1.00 | 37.97 | C |
| ATOM | 109 | O | SER | | 19 | 24.670 | 93.990 | −10.812 | 1.00 | 37.38 | O |
| ATOM | 110 | N | GLY | | 20 | 22.621 | 93.093 | −10.558 | 1.00 | 36.80 | N |
| ATOM | 111 | CA | GLY | | 20 | 22.172 | 94.180 | −9.706 | 1.00 | 34.71 | C |
| ATOM | 112 | C | GLY | | 20 | 21.658 | 95.353 | −10.526 | 1.00 | 34.13 | C |
| ATOM | 113 | O | GLY | | 20 | 21.312 | 96.407 | −9.988 | 1.00 | 34.41 | O |
| ATOM | 114 | N | ARG | | 21 | 21.618 | 95.175 | −11.840 | 1.00 | 32.30 | N |
| ATOM | 115 | CA | ARG | | 21 | 21.134 | 96.221 | −12.727 | 1.00 | 31.06 | C |
| ATOM | 116 | CB | ARG | | 21 | 21.720 | 96.036 | −14.126 | 1.00 | 34.35 | C |
| ATOM | 117 | CG | ARG | | 21 | 21.690 | 97.282 | −15.001 | 1.00 | 38.19 | C |
| ATOM | 118 | CD | ARG | | 21 | 21.821 | 96.898 | −16.466 | 1.00 | 39.57 | C |
| ATOM | 119 | NE | ARG | | 21 | 22.731 | 95.771 | −16.636 | 1.00 | 40.48 | N |
| ATOM | 120 | CZ | ARG | | 21 | 22.721 | 94.950 | −17.681 | 1.00 | 40.27 | C |
| ATOM | 121 | NH1 | ARG | | 21 | 21.849 | 95.127 | −18.668 | 1.00 | 37.17 | N |
| ATOM | 122 | NH2 | ARG | | 21 | 23.573 | 93.933 | −17.723 | 1.00 | 41.07 | N |
| ATOM | 123 | C | ARG | | 21 | 19.617 | 96.088 | −12.786 | 1.00 | 28.79 | C |
| ATOM | 124 | O | ARG | | 21 | 19.083 | 94.977 | −12.759 | 1.00 | 26.98 | O |
| ATOM | 125 | N | VAL | | 22 | 18.921 | 97.213 | −12.865 | 1.00 | 26.13 | N |
| ATOM | 126 | CA | VAL | | 22 | 17.468 | 97.170 | −12.920 | 1.00 | 26.11 | C |
| ATOM | 127 | CB | VAL | | 22 | 16.850 | 98.568 | −12.664 | 1.00 | 27.26 | C |
| ATOM | 128 | CG1 | VAL | | 22 | 17.393 | 99.149 | −11.360 | 1.00 | 28.88 | C |
| ATOM | 129 | CG2 | VAL | | 22 | 17.148 | 99.495 | −13.826 | 1.00 | 26.94 | C |
| ATOM | 130 | C | VAL | | 22 | 16.977 | 96.665 | −14.275 | 1.00 | 23.64 | C |
| ATOM | 131 | O | VAL | | 22 | 17.671 | 96.777 | −15.288 | 1.00 | 22.42 | O |
| ATOM | 132 | N | TYR | | 23 | 15.782 | 96.088 | −14.280 | 1.00 | 23.86 | N |
| ATOM | 133 | CA | TYR | | 23 | 15.173 | 95.610 | −15.511 | 1.00 | 23.22 | C |
| ATOM | 134 | CB | TYR | | 23 | 15.587 | 94.165 | −15.816 | 1.00 | 23.13 | C |
| ATOM | 135 | CG | TYR | | 23 | 15.010 | 93.094 | −14.918 | 1.00 | 24.78 | C |
| ATOM | 136 | CD1 | TYR | | 23 | 13.875 | 92.376 | −15.298 | 1.00 | 25.93 | C |
| ATOM | 137 | CE1 | TYR | | 23 | 13.381 | 91.339 | −14.510 | 1.00 | 27.38 | C |
| ATOM | 138 | CD2 | TYR | | 23 | 15.634 | 92.755 | −13.719 | 1.00 | 27.24 | C |
| ATOM | 139 | CE2 | TYR | | 23 | 15.151 | 91.725 | −12.922 | 1.00 | 29.04 | C |
| ATOM | 140 | CZ | TYR | | 23 | 14.026 | 91.019 | −13.324 | 1.00 | 29.80 | C |
| ATOM | 141 | OH | TYR | | 23 | 13.558 | 89.990 | −12.543 | 1.00 | 32.22 | O |
| ATOM | 142 | C | TYR | | 23 | 13.672 | 95.728 | −15.323 | 1.00 | 24.27 | C |
| ATOM | 143 | O | TYR | | 23 | 13.213 | 96.125 | −14.250 | 1.00 | 23.22 | O |
| ATOM | 144 | N | TYR | | 24 | 12.908 | 95.407 | −16.359 | 1.00 | 23.73 | N |
| ATOM | 145 | CA | TYR | | 24 | 11.462 | 95.509 | −16.267 | 1.00 | 25.12 | C |
| ATOM | 146 | CB | TYR | | 24 | 10.954 | 96.574 | −17.240 | 1.00 | 28.83 | C |
| ATOM | 147 | CG | TYR | | 24 | 11.623 | 97.908 | −17.002 | 1.00 | 34.51 | C |
| ATOM | 148 | CD1 | TYR | | 24 | 12.932 | 98.140 | −17.433 | 1.00 | 35.79 | C |
| ATOM | 149 | CE1 | TYR | | 24 | 13.590 | 99.331 | −17.135 | 1.00 | 38.57 | C |
| ATOM | 150 | CD2 | TYR | | 24 | 10.985 | 98.908 | −16.269 | 1.00 | 36.92 | C |
| ATOM | 151 | CE2 | TYR | | 24 | 11.635 | 100.107 | −15.965 | 1.00 | 39.55 | C |
| ATOM | 152 | CZ | TYR | | 24 | 12.936 | 100.308 | −16.401 | 1.00 | 40.27 | C |
| ATOM | 153 | OH | TYR | | 24 | 13.584 | 101.485 | −16.099 | 1.00 | 43.04 | O |
| ATOM | 154 | C | TYR | | 24 | 10.802 | 94.169 | −16.526 | 1.00 | 23.31 | C |
| ATOM | 155 | O | TYR | | 24 | 11.234 | 93.405 | −17.377 | 1.00 | 19.10 | O |
| ATOM | 156 | N | PHE | | 25 | 9.756 | 93.898 | −15.758 | 1.00 | 23.61 | N |
| ATOM | 157 | CA | PHE | | 25 | 9.013 | 92.654 | −15.845 | 1.00 | 23.90 | C |
| ATOM | 158 | CB | PHE | | 25 | 9.235 | 91.852 | −14.560 | 1.00 | 26.26 | C |
| ATOM | 159 | CG | PHE | | 25 | 8.345 | 90.657 | −14.426 | 1.00 | 28.41 | C |
| ATOM | 160 | CD1 | PHE | | 25 | 8.520 | 89.542 | −15.247 | 1.00 | 29.55 | C |
| ATOM | 161 | CD2 | PHE | | 25 | 7.317 | 90.647 | −13.485 | 1.00 | 29.28 | C |
| ATOM | 162 | CE1 | PHE | | 25 | 7.679 | 88.427 | −15.135 | 1.00 | 29.82 | C |
| ATOM | 163 | CE2 | PHE | | 25 | 6.471 | 89.540 | −13.364 | 1.00 | 31.45 | C |
| ATOM | 164 | CZ | PHE | | 25 | 6.654 | 88.430 | −14.194 | 1.00 | 30.08 | C |
| ATOM | 165 | C | PHE | | 25 | 7.543 | 93.013 | −15.987 | 1.00 | 24.35 | C |
| ATOM | 166 | O | PHE | | 25 | 7.084 | 94.005 | −15.415 | 1.00 | 25.24 | O |
| ATOM | 167 | N | ASN | | 26 | 6.815 | 92.212 | −16.756 | 1.00 | 23.43 | N |
| ATOM | 168 | CA | ASN | | 26 | 5.385 | 92.426 | −16.971 | 1.00 | 22.40 | C |
| ATOM | 169 | CB | ASN | | 26 | 5.094 | 92.543 | −18.471 | 1.00 | 21.86 | C |
| ATOM | 170 | CG | ASN | | 26 | 3.644 | 92.874 | −18.760 | 1.00 | 20.05 | C |
| ATOM | 171 | OD1 | ASN | | 26 | 2.748 | 92.101 | −18.440 | 1.00 | 18.93 | O |
| ATOM | 172 | ND2 | ASN | | 26 | 3.410 | 94.029 | −19.370 | 1.00 | 21.42 | N |
| ATOM | 173 | C | ASN | | 26 | 4.677 | 91.213 | −16.377 | 1.00 | 19.54 | C |
| ATOM | 174 | O | ASN | | 26 | 4.905 | 90.081 | −16.805 | 1.00 | 18.99 | O |
| ATOM | 175 | N | HIS | | 27 | 3.822 | 91.439 | −15.385 | 1.00 | 18.51 | N |
| ATOM | 176 | CA | HIS | | 27 | 3.143 | 90.323 | −14.731 | 1.00 | 19.95 | C |
| ATOM | 177 | CB | HIS | | 27 | 2.727 | 90.719 | −13.310 | 1.00 | 20.01 | C |
| ATOM | 178 | CG | HIS | | 27 | 1.832 | 91.916 | −13.253 | 1.00 | 22.73 | C |
| ATOM | 179 | CD2 | HIS | | 27 | 2.101 | 93.217 | −12.995 | 1.00 | 24.62 | C |
| ATOM | 180 | ND1 | HIS | | 27 | 0.476 | 91.845 | −13.492 | 1.00 | 25.87 | N |
| ATOM | 181 | CE1 | HIS | | 27 | −0.052 | 93.051 | −13.384 | 1.00 | 27.16 | C |
| ATOM | 182 | NE2 | HIS | | 27 | 0.913 | 93.903 | −13.084 | 1.00 | 27.76 | N |
| ATOM | 183 | C | HIS | | 27 | 1.944 | 89.747 | −15.480 | 1.00 | 19.02 | C |
| ATOM | 184 | O | HIS | | 27 | 1.305 | 88.817 | −14.997 | 1.00 | 21.28 | O |
| ATOM | 185 | N | ILE | | 28 | 1.639 | 90.294 | −16.650 | 1.00 | 20.60 | N |
| ATOM | 186 | CA | ILE | | 28 | 0.525 | 89.788 | −17.444 | 1.00 | 21.05 | C |
| ATOM | 187 | CB | ILE | | 28 | −0.316 | 90.935 | −18.051 | 1.00 | 22.57 | C |
| ATOM | 188 | CG2 | ILE | | 28 | −1.479 | 90.350 | −18.852 | 1.00 | 20.76 | C |
| ATOM | 189 | CG1 | ILE | | 28 | −0.835 | 91.851 | −16.942 | 1.00 | 22.09 | C |
| ATOM | 190 | CD1 | ILE | | 28 | −1.473 | 93.137 | −17.451 | 1.00 | 23.01 | C |
| ATOM | 191 | C | ILE | | 28 | 1.058 | 88.923 | −18.588 | 1.00 | 20.36 | C |
| ATOM | 192 | O | ILE | | 28 | 0.490 | 87.876 | −18.897 | 1.00 | 21.46 | O |
| ATOM | 193 | N | THR | | 29 | 2.158 | 89.354 | −19.205 | 1.00 | 19.41 | N |
| ATOM | 194 | CA | THR | | 29 | 2.749 | 88.611 | −20.322 | 1.00 | 18.44 | C |
| ATOM | 195 | CB | THR | | 29 | 3.245 | 89.548 | −21.431 | 1.00 | 16.48 | C |
| ATOM | 196 | OG1 | THR | | 29 | 4.380 | 90.277 | −20.947 | 1.00 | 15.52 | O |
| ATOM | 197 | CG2 | THR | | 29 | 2.162 | 90.528 | −21.838 | 1.00 | 17.51 | C |
| ATOM | 198 | C | THR | | 29 | 3.954 | 87.787 | −19.892 | 1.00 | 17.66 | C |
| ATOM | 199 | O | THR | | 29 | 4.407 | 86.913 | −20.635 | 1.00 | 15.04 | O |
| ATOM | 200 | N | ASN | | 30 | 4.469 | 88.083 | −18.699 | 1.00 | 18.86 | N |
| ATOM | 201 | CA | ASN | | 30 | 5.646 | 87.411 | −18.150 | 1.00 | 19.21 | C |
| ATOM | 202 | CB | ASN | | 30 | 5.418 | 85.898 | −18.075 | 1.00 | 18.37 | C |
| ATOM | 203 | CG | ASN | | 30 | 4.631 | 85.491 | −16.828 | 1.00 | 21.15 | C |
| ATOM | 204 | OD1 | ASN | | 30 | 3.985 | 84.436 | −16.795 | 1.00 | 20.51 | O |
| ATOM | 205 | ND2 | ASN | | 30 | 4.696 | 86.325 | −15.792 | 1.00 | 18.80 | N |
| ATOM | 206 | C | ASN | | 30 | 6.911 | 87.735 | −18.949 | 1.00 | 18.18 | C |
| ATOM | 207 | O | ASN | | 30 | 7.920 | 87.035 | −18.857 | 1.00 | 18.36 | O |
| ATOM | 208 | N | ALA | | 31 | 6.859 | 88.797 | −19.740 | 1.00 | 18.07 | N |
| ATOM | 209 | CA | ALA | | 31 | 8.042 | 89.188 | −20.497 | 1.00 | 18.58 | C |
| ATOM | 210 | CB | ALA | | 31 | 7.654 | 90.066 | −21.680 | 1.00 | 20.05 | C |
| ATOM | 211 | C | ALA | | 31 | 8.974 | 89.960 | −19.562 | 1.00 | 19.63 | C |
| ATOM | 212 | O | ALA | | 31 | 8.538 | 90.550 | −18.561 | 1.00 | 19.30 | O |
| ATOM | 213 | N | SER | | 32 | 10.263 | 89.944 | −19.875 | 1.00 | 19.74 | N |
| ATOM | 214 | CA | SER | | 32 | 11.231 | 90.685 | −19.082 | 1.00 | 20.22 | C |
| ATOM | 215 | CB | SER | | 32 | 11.840 | 89.801 | −17.987 | 1.00 | 19.25 | C |
| ATOM | 216 | OG | SER | | 32 | 12.328 | 88.583 | −18.505 | 1.00 | 24.38 | O |
| ATOM | 217 | C | SER | | 32 | 12.301 | 91.219 | −20.024 | 1.00 | 21.37 | C |
| ATOM | 218 | O | SER | | 32 | 12.672 | 90.563 | −20.996 | 1.00 | 21.28 | O |
| ATOM | 219 | N | GLN | | 33 | 12.773 | 92.429 | −19.755 | 1.00 | 23.24 | N |
| ATOM | 220 | CA | GLN | | 33 | 13.788 | 93.034 | −20.600 | 1.00 | 24.72 | C |
| ATOM | 221 | CB | GLN | | 33 | 13.131 | 93.645 | −21.835 | 1.00 | 25.90 | C |
| ATOM | 222 | CG | GLN | | 33 | 12.102 | 94.722 | −21.529 | 1.00 | 29.80 | C |
| ATOM | 223 | CD | GLN | | 33 | 11.227 | 95.046 | −22.729 | 1.00 | 33.65 | C |
| ATOM | 224 | OE1 | GLN | | 33 | 10.470 | 96.017 | −22.718 | 1.00 | 34.96 | O |
| ATOM | 225 | NE2 | GLN | | 33 | 11.318 | 94.222 | −23.769 | 1.00 | 34.57 | N |
| ATOM | 226 | C | GLN | | 33 | 14.551 | 94.103 | −19.834 | 1.00 | 25.35 | C |
| ATOM | 227 | O | GLN | | 33 | 14.092 | 94.576 | −18.795 | 1.00 | 24.37 | O |
| ATOM | 228 | N | TRP | | 34 | 15.708 | 94.487 | −20.366 | 1.00 | 26.89 | N |
| ATOM | 229 | CA | TRP | | 34 | 16.553 | 95.498 | −19.741 | 1.00 | 27.75 | C |
| ATOM | 230 | CB | TRP | | 34 | 17.976 | 95.412 | −20.306 | 1.00 | 26.26 | C |
| ATOM | 231 | CG | TRP | | 34 | 18.699 | 94.156 | −19.913 | 1.00 | 24.74 | C |
| ATOM | 232 | CD2 | TRP | | 34 | 19.033 | 93.739 | −18.582 | 1.00 | 25.26 | C |
| ATOM | 233 | CE2 | TRP | | 34 | 19.695 | 92.495 | −18.687 | 1.00 | 26.19 | C |
| ATOM | 234 | CE3 | TRP | | 34 | 18.836 | 94.295 | −17.310 | 1.00 | 24.96 | C |
| ATOM | 235 | CD1 | TRP | | 34 | 19.158 | 93.179 | −20.746 | 1.00 | 24.73 | C |
| ATOM | 236 | NE1 | TRP | | 34 | 19.758 | 92.179 | −20.019 | 1.00 | 26.44 | N |
| ATOM | 237 | CZ2 | TRP | | 34 | 20.163 | 91.792 | −17.567 | 1.00 | 26.97 | C |
| ATOM | 238 | CZ3 | TRP | | 34 | 19.301 | 93.598 | −16.194 | 1.00 | 26.62 | C |
| ATOM | 239 | CH2 | TRP | | 34 | 19.957 | 92.358 | −16.333 | 1.00 | 26.96 | C |
| ATOM | 240 | C | TRP | | 34 | 16.028 | 96.921 | −19.899 | 1.00 | 31.05 | C |
| ATOM | 241 | O | TRP | | 34 | 16.115 | 97.724 | −18.969 | 1.00 | 29.24 | O |
| ATOM | 242 | N | GLU | | 35 | 15.479 | 97.240 | −21.068 | 1.00 | 35.17 | N |
| ATOM | 243 | CA | GLU | | 35 | 14.967 | 98.587 | −21.302 | 1.00 | 40.03 | C |
| ATOM | 244 | CB | GLU | | 35 | 14.898 | 98.890 | −22.792 | 1.00 | 42.14 | C |
| ATOM | 245 | CG | GLU | | 35 | 16.231 | 99.024 | −23.476 | 1.00 | 46.01 | C |
| ATOM | 246 | CD | GLU | | 35 | 16.142 | 99.926 | −24.684 | 1.00 | 47.87 | C |
| ATOM | 247 | OE1 | GLU | | 35 | 16.985 | 99.786 | −25.593 | 1.00 | 49.04 | O |
| ATOM | 248 | OE2 | GLU | | 35 | 15.229 | 100.787 | −24.715 | 1.00 | 49.18 | O |
| ATOM | 249 | C | GLU | | 35 | 13.601 | 98.878 | −20.707 | 1.00 | 43.21 | C |
| ATOM | 250 | O | GLU | | 35 | 12.782 | 97.981 | −20.513 | 1.00 | 42.38 | O |
| ATOM | 251 | N | ARG | | 36 | 13.366 | 100.155 | −20.427 | 1.00 | 47.08 | N |
| ATOM | 252 | CA | ARG | | 36 | 12.094 | 100.599 | −19.882 | 1.00 | 51.80 | C |
| ATOM | 253 | CB | ARG | | 36 | 12.237 | 101.998 | −19.280 | 1.00 | 53.12 | C |
| ATOM | 254 | CG | ARG | | 36 | 11.031 | 102.449 | −18.478 | 1.00 | 56.83 | C |
| ATOM | 255 | CD | ARG | | 36 | 11.378 | 103.617 | −17.568 | 1.00 | 58.92 | C |
| ATOM | 256 | NE | ARG | | 36 | 10.266 | 103.966 | −16.690 | 1.00 | 61.98 | N |
| ATOM | 257 | CZ | ARG | | 36 | 10.352 | 104.816 | −15.672 | 1.00 | 63.63 | C |
| ATOM | 258 | NH1 | ARG | | 36 | 11.506 | 105.413 | −15.398 | 1.00 | 65.04 | N |
| ATOM | 259 | NH2 | ARG | | 36 | 9.284 | 105.067 | −14.925 | 1.00 | 64.32 | N |
| ATOM | 260 | C | ARG | | 36 | 11.134 | 100.613 | −21.065 | 1.00 | 53.82 | C |
| ATOM | 261 | O | ARG | | 36 | 11.356 | 101.323 | −22.048 | 1.00 | 54.14 | O |
| ATOM | 262 | N | PRO | | 37 | 10.056 | 99.819 | −20.988 | 1.00 | 55.67 | N |
| ATOM | 263 | CD | PRO | | 37 | 9.601 | 99.102 | −19.784 | 1.00 | 55.91 | C |
| ATOM | 264 | CA | PRO | | 37 | 9.054 | 99.725 | −22.054 | 1.00 | 57.14 | C |
| ATOM | 265 | CB | PRO | | 37 | 8.084 | 98.680 | −21.512 | 1.00 | 56.96 | C |
| ATOM | 266 | CG | PRO | | 37 | 8.118 | 98.949 | −20.044 | 1.00 | 56.31 | C |
| ATOM | 267 | C | PRO | | 37 | 8.358 | 101.037 | −22.407 | 1.00 | 58.98 | C |
| ATOM | 268 | O | PRO | | 37 | 8.079 | 101.864 | −21.532 | 1.00 | 58.84 | O |
| ATOM | 269 | N | SER | | 38 | 8.080 | 101.209 | −23.699 | 1.00 | 60.46 | N |
| ATOM | 270 | CA | SER | | 38 | 7.403 | 102.396 | −24.220 | 1.00 | 62.00 | C |
| ATOM | 271 | CB | SER | | 38 | 5.886 | 102.205 | −24.140 | 1.00 | 62.10 | C |
| ATOM | 272 | OG | SER | | 38 | 5.496 | 100.982 | −24.740 | 1.00 | 62.82 | O |
| ATOM | 273 | C | SER | | 38 | 7.797 | 103.672 | −23.480 | 1.00 | 62.50 | C |
| ATOM | 274 | O | SER | | 38 | 6.987 | 104.257 | −22.757 | 1.00 | 63.00 | O |
| ATOM | 275 | N | GLU | | 51 | −7.299 | 100.145 | −25.882 | 1.00 | 54.73 | N |
| ATOM | 276 | CA | GLU | | 51 | −6.547 | 99.038 | −26.459 | 1.00 | 54.16 | C |
| ATOM | 277 | CB | GLU | | 51 | −6.525 | 97.850 | −25.492 | 1.00 | 53.49 | C |
| ATOM | 278 | CG | GLU | | 51 | −7.802 | 97.023 | −25.496 | 1.00 | 52.61 | C |
| ATOM | 279 | CD | GLU | | 51 | −7.781 | 95.896 | −24.480 | 1.00 | 51.66 | C |
| ATOM | 280 | OE1 | GLU | | 51 | −8.699 | 95.054 | −24.519 | 1.00 | 51.27 | O |
| ATOM | 281 | OE2 | GLU | | 51 | −6.855 | 95.854 | −23.640 | 1.00 | 50.71 | O |
| ATOM | 282 | C | GLU | | 51 | −7.195 | 98.617 | −27.776 | 1.00 | 54.07 | C |
| ATOM | 283 | O | GLU | | 51 | −8.335 | 98.989 | −28.063 | 1.00 | 54.46 | O |
| ATOM | 284 | N | PRO | | 52 | −6.474 | 97.833 | −28.597 | 1.00 | 53.57 | N |
| ATOM | 285 | CD | PRO | | 52 | −5.070 | 97.415 | −28.447 | 1.00 | 53.61 | C |
| ATOM | 286 | CA | PRO | | 52 | −7.014 | 97.378 | −29.883 | 1.00 | 51.72 | C |
| ATOM | 287 | CB | PRO | | 52 | −5.853 | 96.590 | −30.488 | 1.00 | 52.84 | C |
| ATOM | 288 | CG | PRO | | 52 | −4.646 | 97.240 | −29.881 | 1.00 | 53.98 | C |
| ATOM | 289 | C | PRO | | 52 | −8.252 | 96.505 | −29.699 | 1.00 | 49.84 | C |
| ATOM | 290 | O | PRO | | 52 | −8.416 | 95.855 | −28.666 | 1.00 | 49.91 | O |
| ATOM | 291 | N | ALA | | 53 | −9.118 | 96.498 | −30.706 | 1.00 | 46.77 | N |
| ATOM | 292 | CA | ALA | | 53 | −10.330 | 95.694 | −30.665 | 1.00 | 43.86 | C |
| ATOM | 293 | CB | ALA | | 53 | −11.320 | 96.185 | −31.712 | 1.00 | 44.36 | C |
| ATOM | 294 | C | ALA | | 53 | −9.933 | 94.256 | −30.956 | 1.00 | 41.48 | C |
| ATOM | 295 | O | ALA | | 53 | −10.553 | 93.312 | −30.465 | 1.00 | 39.69 | O |
| ATOM | 296 | N | ARG | | 54 | −8.883 | 94.104 | −31.758 | 1.00 | 39.02 | N |
| ATOM | 297 | CA | ARG | | 54 | −8.384 | 92.789 | −32.138 | 1.00 | 36.56 | C |
| ATOM | 298 | CB | ARG | | 54 | −8.983 | 92.369 | −33.485 | 1.00 | 39.18 | C |
| ATOM | 299 | CG | ARG | | 54 | −10.502 | 92.387 | −33.539 | 1.00 | 42.60 | C |
| ATOM | 300 | CD | ARG | | 54 | −11.011 | 92.344 | −34.979 | 1.00 | 44.45 | C |
| ATOM | 301 | NE | ARG | | 54 | −10.748 | 91.071 | −35.645 | 1.00 | 46.01 | N |
| ATOM | 302 | CZ | ARG | | 54 | −11.250 | 89.904 | −35.253 | 1.00 | 48.61 | C |
| ATOM | 303 | NH1 | ARG | | 54 | −12.044 | 89.842 | −34.190 | 1.00 | 49.20 | N |
| ATOM | 304 | NH2 | ARG | | 54 | −10.972 | 88.797 | −35.933 | 1.00 | 48.94 | N |
| ATOM | 305 | C | ARG | | 54 | −6.863 | 92.790 | −32.259 | 1.00 | 33.22 | C |
| ATOM | 306 | O | ARG | | 54 | −6.243 | 93.821 | −32.525 | 1.00 | 31.35 | O |
| ATOM | 307 | N | VAL | | 55 | −6.269 | 91.623 | −32.048 | 1.00 | 29.61 | N |
| ATOM | 308 | CA | VAL | | 55 | −4.828 | 91.461 | −32.178 | 1.00 | 26.44 | C |
| ATOM | 309 | CB | VAL | | 55 | −4.104 | 91.355 | −30.801 | 1.00 | 25.08 | C |
| ATOM | 310 | CG1 | VAL | | 55 | −4.372 | 92.595 | −29.966 | 1.00 | 24.38 | C |
| ATOM | 311 | CG2 | VAL | | 55 | −4.559 | 90.100 | −30.065 | 1.00 | 25.34 | C |
| ATOM | 312 | C | VAL | | 55 | −4.625 | 90.151 | −32.910 | 1.00 | 25.04 | C |
| ATOM | 313 | O | VAL | | 55 | −5.523 | 89.308 | −32.947 | 1.00 | 24.96 | O |
| ATOM | 314 | N | ARG | | 56 | −3.454 | 89.988 | −33.511 | 1.00 | 22.94 | N |
| ATOM | 315 | CA | ARG | | 56 | −3.127 | 88.755 | −34.202 | 1.00 | 21.84 | C |
| ATOM | 316 | CB | ARG | | 56 | −2.846 | 89.023 | −35.683 | 1.00 | 22.77 | C |
| ATOM | 317 | CG | ARG | | 56 | −2.554 | 87.763 | −36.478 | 1.00 | 24.73 | C |
| ATOM | 318 | CD | ARG | | 56 | −2.221 | 88.070 | −37.934 | 1.00 | 24.89 | C |
| ATOM | 319 | NE | ARG | | 56 | −1.982 | 86.848 | −38.696 | 1.00 | 27.57 | N |
| ATOM | 320 | CZ | ARG | | 56 | −1.732 | 86.819 | −40.000 | 1.00 | 30.02 | C |
| ATOM | 321 | NH1 | ARG | | 56 | −1.688 | 87.950 | −40.692 | 1.00 | 28.85 | N |
| ATOM | 322 | NH2 | ARG | | 56 | −1.528 | 85.660 | −40.614 | 1.00 | 29.59 | N |
| ATOM | 323 | C | ARG | | 56 | −1.877 | 88.204 | −33.521 | 1.00 | 20.99 | C |
| ATOM | 324 | O | ARG | | 56 | −0.914 | 88.940 | −33.299 | 1.00 | 19.81 | O |
| ATOM | 325 | N | CYS | | 57 | −1.896 | 86.920 | −33.177 | 1.00 | 19.41 | N |
| ATOM | 326 | CA | CYS | | 57 | −0.745 | 86.304 | −32.521 | 1.00 | 19.30 | C |
| ATOM | 327 | CB | CYS | | 57 | −0.989 | 86.145 | −31.012 | 1.00 | 18.77 | C |
| ATOM | 328 | SG | CYS | | 57 | −1.263 | 87.657 | −30.087 | 1.00 | 19.47 | S |
| ATOM | 329 | C | CYS | | 57 | −0.417 | 84.933 | −33.060 | 1.00 | 19.50 | C |
| ATOM | 330 | O | CYS | | 57 | −1.250 | 84.267 | −33.675 | 1.00 | 20.50 | O |
| ATOM | 331 | N | SER | | 58 | 0.822 | 84.524 | −32.825 | 1.00 | 17.55 | N |
| ATOM | 332 | CA | SER | | 58 | 1.286 | 83.196 | −33.173 | 1.00 | 17.69 | C |
| ATOM | 333 | CB | SER | | 58 | 2.499 | 83.242 | −34.108 | 1.00 | 18.00 | C |
| ATOM | 334 | OG | SER | | 58 | 2.137 | 83.702 | −35.390 | 1.00 | 18.64 | O |
| ATOM | 335 | C | SER | | 58 | 1.725 | 82.697 | −31.805 | 1.00 | 18.37 | C |
| ATOM | 336 | O | SER | | 58 | 1.940 | 83.503 | −30.889 | 1.00 | 18.47 | O |
| ATOM | 337 | N | HIS | | 59 | 1.850 | 81.390 | −31.641 | 1.00 | 17.51 | N |
| ATOM | 338 | CA | HIS | | 59 | 2.294 | 80.872 | −30.360 | 1.00 | 17.57 | C |
| ATOM | 339 | CB | HIS | | 59 | 1.118 | 80.810 | −29.359 | 1.00 | 18.76 | C |
| ATOM | 340 | CG | HIS | | 59 | 0.260 | 79.587 | −29.488 | 1.00 | 20.38 | C |
| ATOM | 341 | CD2 | HIS | | 59 | −0.139 | 78.681 | −28.564 | 1.00 | 20.36 | C |
| ATOM | 342 | ND1 | HIS | | 59 | −0.312 | 79.194 | −30.680 | 1.00 | 22.08 | N |
| ATOM | 343 | CE1 | HIS | | 59 | −1.025 | 78.097 | −30.485 | 1.00 | 20.02 | C |
| ATOM | 344 | NE2 | HIS | | 59 | −0.937 | 77.766 | −29.210 | 1.00 | 24.12 | N |
| ATOM | 345 | C | HIS | | 59 | 2.955 | 79.508 | −30.501 | 1.00 | 17.86 | C |
| ATOM | 346 | O | HIS | | 59 | 2.825 | 78.829 | −31.526 | 1.00 | 16.44 | O |
| ATOM | 347 | N | LEU | | 60 | 3.701 | 79.133 | −29.474 | 1.00 | 17.48 | N |
| ATOM | 348 | CA | LEU | | 60 | 4.367 | 77.847 | −29.436 | 1.00 | 18.71 | C |
| ATOM | 349 | CB | LEU | | 60 | 5.887 | 78.023 | −29.382 | 1.00 | 17.85 | C |
| ATOM | 350 | CG | LEU | | 60 | 6.736 | 76.753 | −29.516 | 1.00 | 17.50 | C |
| ATOM | 351 | CD1 | LEU | | 60 | 8.168 | 77.146 | −29.868 | 1.00 | 18.29 | C |
| ATOM | 352 | CD2 | LEU | | 60 | 6.697 | 75.945 | −28.225 | 1.00 | 17.89 | C |
| ATOM | 353 | C | LEU | | 60 | 3.854 | 77.248 | −28.133 | 1.00 | 21.30 | C |
| ATOM | 354 | O | LEU | | 60 | 4.044 | 77.824 | −27.058 | 1.00 | 20.24 | O |
| ATOM | 355 | N | LEU | | 61 | 3.185 | 76.108 | −28.236 | 1.00 | 20.45 | N |
| ATOM | 356 | CA | LEU | | 61 | 2.624 | 75.448 | −27.065 | 1.00 | 20.60 | C |
| ATOM | 357 | CB | LEU | | 61 | 1.149 | 75.089 | −27.308 | 1.00 | 21.94 | C |
| ATOM | 358 | CG | LEU | | 61 | 0.511 | 74.157 | −26.263 | 1.00 | 21.71 | C |
| ATOM | 359 | CD1 | LEU | | 61 | 0.524 | 74.838 | −24.897 | 1.00 | 22.57 | C |
| ATOM | 360 | CD2 | LEU | | 61 | −0.915 | 73.803 | −26.665 | 1.00 | 21.58 | C |
| ATOM | 361 | C | LEU | | 61 | 3.377 | 74.184 | −26.700 | 1.00 | 19.71 | C |
| ATOM | 362 | O | LEU | | 61 | 3.672 | 73.352 | −27.556 | 1.00 | 18.48 | O |
| ATOM | 363 | N | VAL | | 62 | 3.710 | 74.064 | −25.421 | 1.00 | 20.78 | N |
| ATOM | 364 | CA | VAL | | 62 | 4.372 | 72.874 | −24.928 | 1.00 | 21.01 | C |
| ATOM | 365 | CB | VAL | | 62 | 5.713 | 73.180 | −24.260 | 1.00 | 21.01 | C |
| ATOM | 366 | CG1 | VAL | | 62 | 6.249 | 71.917 | −23.593 | 1.00 | 22.48 | C |
| ATOM | 367 | CG2 | VAL | | 62 | 6.702 | 73.672 | −25.303 | 1.00 | 20.98 | C |
| ATOM | 368 | C | VAL | | 62 | 3.405 | 72.316 | −23.898 | 1.00 | 23.01 | C |
| ATOM | 369 | O | VAL | | 62 | 3.182 | 72.919 | −22.845 | 1.00 | 20.35 | O |
| ATOM | 370 | N | LYS | | 63 | 2.795 | 71.185 | −24.234 | 1.00 | 22.70 | N |
| ATOM | 371 | CA | LYS | | 63 | 1.843 | 70.554 | −23.338 | 1.00 | 25.68 | C |
| ATOM | 372 | CB | LYS | | 63 | 0.885 | 69.656 | −24.131 | 1.00 | 26.95 | C |
| ATOM | 373 | CG | LYS | | 63 | −0.159 | 70.406 | −24.942 | 1.00 | 29.77 | C |
| ATOM | 374 | CD | LYS | | 63 | −1.162 | 69.434 | −25.566 | 1.00 | 31.46 | C |
| ATOM | 375 | CE | LYS | | 63 | −2.341 | 70.168 | −26.180 | 1.00 | 32.39 | C |
| ATOM | 376 | NZ | LYS | | 63 | −3.356 | 69.215 | −26.717 | 1.00 | 32.43 | N |
| ATOM | 377 | C | LYS | | 63 | 2.570 | 69.720 | −22.295 | 1.00 | 26.46 | C |
| ATOM | 378 | O | LYS | | 63 | 3.765 | 69.439 | −22.424 | 1.00 | 25.58 | O |
| ATOM | 379 | N | HIS | | 64 | 1.839 | 69.343 | −21.252 | 1.00 | 27.05 | N |
| ATOM | 380 | CA | HIS | | 64 | 2.378 | 68.508 | −20.189 | 1.00 | 28.81 | C |
| ATOM | 381 | CB | HIS | | 64 | 2.920 | 69.364 | −19.035 | 1.00 | 26.98 | C |
| ATOM | 382 | CG | HIS | | 64 | 1.959 | 70.395 | −18.535 | 1.00 | 28.06 | C |
| ATOM | 383 | CD2 | HIS | | 64 | 1.994 | 71.748 | −18.597 | 1.00 | 27.80 | C |
| ATOM | 384 | ND1 | HIS | | 64 | 0.795 | 70.072 | −17.871 | 1.00 | 27.74 | N |
| ATOM | 385 | CE1 | HIS | | 64 | 0.155 | 71.181 | −17.546 | 1.00 | 30.15 | C |
| ATOM | 386 | NE2 | HIS | | 64 | 0.861 | 72.212 | −17.975 | 1.00 | 29.96 | N |
| ATOM | 387 | C | HIS | | 64 | 1.272 | 67.571 | −19.699 | 1.00 | 30.37 | C |
| ATOM | 388 | O | HIS | | 64 | 0.114 | 67.695 | −20.113 | 1.00 | 29.45 | O |
| ATOM | 389 | N | SER | | 65 | 1.633 | 66.640 | −18.821 | 1.00 | 31.38 | N |
| ATOM | 390 | CA | SER | | 65 | 0.683 | 65.660 | −18.305 | 1.00 | 31.46 | C |
| ATOM | 391 | CB | SER | | 65 | 1.375 | 64.753 | −17.287 | 1.00 | 32.89 | C |
| ATOM | 392 | OG | SER | | 65 | 1.779 | 65.486 | −16.147 | 1.00 | 35.80 | O |
| ATOM | 393 | C | SER | | 65 | −0.606 | 66.225 | −17.704 | 1.00 | 31.05 | C |
| ATOM | 394 | O | SER | | 65 | −1.604 | 65.513 | −17.621 | 1.00 | 31.26 | O |
| ATOM | 395 | N | GLN | | 66 | −0.607 | 67.487 | −17.283 | 1.00 | 29.89 | N |
| ATOM | 396 | CA | GLN | | 66 | −1.825 | 68.065 | −16.716 | 1.00 | 29.89 | C |
| ATOM | 397 | CB | GLN | | 66 | −1.513 | 68.887 | −15.455 | 1.00 | 31.36 | C |
| ATOM | 398 | CG | GLN | | 66 | −1.179 | 68.040 | −14.229 | 1.00 | 32.82 | C |
| ATOM | 399 | CD | GLN | | 66 | −1.109 | 68.855 | −12.943 | 1.00 | 36.67 | C |
| ATOM | 400 | OE1 | GLN | | 66 | −2.117 | 69.400 | −12.478 | 1.00 | 33.97 | O |
| ATOM | 401 | NE2 | GLN | | 66 | 0.087 | 68.941 | −12.362 | 1.00 | 36.73 | N |
| ATOM | 402 | C | GLN | | 66 | −2.633 | 68.915 | −17.700 | 1.00 | 30.12 | C |
| ATOM | 403 | O | GLN | | 66 | −3.586 | 69.590 | −17.305 | 1.00 | 31.23 | O |
| ATOM | 404 | N | SER | | 67 | −2.250 | 68.896 | −18.975 | 1.00 | 30.74 | N |
| ATOM | 405 | CA | SER | | 67 | −2.990 | 69.643 | −19.992 | 1.00 | 32.03 | C |
| ATOM | 406 | CB | SER | | 67 | −2.228 | 69.644 | −21.323 | 1.00 | 31.44 | C |
| ATOM | 407 | OG | SER | | 67 | −0.987 | 70.325 | −21.217 | 1.00 | 28.66 | O |
| ATOM | 408 | C | SER | | 67 | −4.326 | 68.917 | −20.166 | 1.00 | 33.77 | C |
| ATOM | 409 | O | SER | | 67 | −4.360 | 67.687 | −20.114 | 1.00 | 33.93 | O |
| ATOM | 410 | N | ARG | | 68 | −5.415 | 69.663 | −20.365 | 1.00 | 35.93 | N |
| ATOM | 411 | CA | ARG | | 68 | −6.733 | 69.044 | −20.534 | 1.00 | 37.58 | C |
| ATOM | 412 | CB | ARG | | 68 | −7.733 | 70.021 | −21.160 | 1.00 | 39.33 | C |
| ATOM | 413 | CG | ARG | | 68 | −8.167 | 71.164 | −20.257 | 1.00 | 43.86 | C |
| ATOM | 414 | CD | ARG | | 68 | −8.673 | 70.656 | −18.911 | 1.00 | 47.04 | C |
| ATOM | 415 | NE | ARG | | 68 | −9.133 | 71.746 | −18.053 | 1.00 | 48.93 | N |
| ATOM | 416 | CZ | ARG | | 68 | −9.237 | 71.660 | −16.730 | 1.00 | 50.45 | C |
| ATOM | 417 | NH1 | ARG | | 68 | −8.909 | 70.533 | −16.108 | 1.00 | 50.38 | N |
| ATOM | 418 | NH2 | ARG | | 68 | −9.669 | 72.699 | −16.028 | 1.00 | 50.61 | N |
| ATOM | 419 | C | ARG | | 68 | −6.634 | 67.806 | −21.412 | 1.00 | 38.24 | C |
| ATOM | 420 | O | ARG | | 68 | −7.174 | 66.748 | −21.083 | 1.00 | 37.80 | O |
| ATOM | 421 | N | ARG | | 69 | −5.935 | 67.947 | −22.531 | 1.00 | 37.54 | N |
| ATOM | 422 | CA | ARG | | 69 | −5.748 | 66.848 | −23.463 | 1.00 | 37.33 | C |
| ATOM | 423 | CB | ARG | | 69 | −6.429 | 67.180 | −24.791 | 1.00 | 40.02 | C |
| ATOM | 424 | CG | ARG | | 69 | −6.077 | 66.241 | −25.928 | 1.00 | 45.02 | C |
| ATOM | 425 | CD | ARG | | 69 | −6.171 | 66.979 | −27.253 | 1.00 | 49.84 | C |
| ATOM | 426 | NE | ARG | | 69 | −5.460 | 66.301 | −28.333 | 1.00 | 52.65 | N |
| ATOM | 427 | CZ | ARG | | 69 | −5.140 | 66.880 | −29.488 | 1.00 | 54.87 | C |
| ATOM | 428 | NH1 | ARG | | 69 | −5.467 | 68.149 | −29.707 | 1.00 | 54.97 | N |
| ATOM | 429 | NH2 | ARG | | 69 | −4.497 | 66.192 | −30.426 | 1.00 | 56.36 | N |
| ATOM | 430 | C | ARG | | 69 | −4.249 | 66.602 | −23.678 | 1.00 | 36.57 | C |
| ATOM | 431 | O | ARG | | 69 | −3.626 | 67.208 | −24.557 | 1.00 | 35.67 | O |
| ATOM | 432 | N | PRO | | 70 | −3.650 | 65.712 | −22.869 | 1.00 | 34.72 | N |
| ATOM | 433 | CD | PRO | | 70 | −4.261 | 64.994 | −21.736 | 1.00 | 35.36 | C |
| ATOM | 434 | CA | PRO | | 70 | −2.222 | 65.393 | −22.977 | 1.00 | 33.32 | C |
| ATOM | 435 | CB | PRO | | 70 | −1.953 | 64.587 | −21.707 | 1.00 | 34.34 | C |
| ATOM | 436 | CG | PRO | | 70 | −3.258 | 63.895 | −21.473 | 1.00 | 34.73 | C |
| ATOM | 437 | C | PRO | | 70 | −1.855 | 64.632 | −24.250 | 1.00 | 31.63 | C |
| ATOM | 438 | O | PRO | | 70 | −1.309 | 63.528 | −24.198 | 1.00 | 31.57 | O |
| ATOM | 439 | N | SER | | 71 | −2.143 | 65.245 | −25.395 | 1.00 | 30.37 | N |
| ATOM | 440 | CA | SER | | 71 | −1.854 | 64.642 | −26.693 | 1.00 | 29.01 | C |
| ATOM | 441 | CB | SER | | 71 | −2.974 | 63.665 | −27.076 | 1.00 | 29.15 | C |
| ATOM | 442 | OG | SER | | 71 | −2.685 | 62.990 | −28.291 | 1.00 | 31.24 | O |
| ATOM | 443 | C | SER | | 71 | −1.765 | 65.740 | −27.748 | 1.00 | 27.74 | C |
| ATOM | 444 | O | SER | | 71 | −2.376 | 66.799 | −27.590 | 1.00 | 28.29 | O |
| ATOM | 445 | N | SER | | 72 | −1.004 | 65.484 | −28.811 | 1.00 | 26.64 | N |
| ATOM | 446 | CA | SER | | 72 | −0.853 | 66.440 | −29.914 | 1.00 | 27.23 | C |
| ATOM | 447 | CB | SER | | 72 | 0.118 | 67.566 | −29.542 | 1.00 | 26.09 | C |
| ATOM | 448 | OG | SER | | 72 | 1.460 | 67.121 | −29.657 | 1.00 | 20.71 | O |
| ATOM | 449 | C | SER | | 72 | −0.312 | 65.721 | −31.141 | 1.00 | 27.44 | C |
| ATOM | 450 | O | SER | | 72 | 0.033 | 64.544 | −31.075 | 1.00 | 27.06 | O |
| ATOM | 451 | N | TRP | | 73 | −0.216 | 66.439 | −32.257 | 1.00 | 28.05 | N |
| ATOM | 452 | CA | TRP | | 73 | 0.296 | 65.852 | −33.487 | 1.00 | 27.76 | C |
| ATOM | 453 | CB | TRP | | 73 | 0.212 | 66.861 | −34.643 | 1.00 | 28.27 | C |
| ATOM | 454 | CG | TRP | | 73 | 1.202 | 67.995 | −34.531 | 1.00 | 27.03 | C |
| ATOM | 455 | CD2 | TRP | | 73 | 2.547 | 68.010 | −35.032 | 1.00 | 26.83 | C |
| ATOM | 456 | CE2 | TRP | | 73 | 3.122 | 69.246 | −34.652 | 1.00 | 24.67 | C |
| ATOM | 457 | CE3 | TRP | | 73 | 3.324 | 67.097 | −35.761 | 1.00 | 26.46 | C |
| ATOM | 458 | CD1 | TRP | | 73 | 1.020 | 69.190 | −33.888 | 1.00 | 26.62 | C |
| ATOM | 459 | NE1 | TRP | | 73 | 2.170 | 69.945 | −33.957 | 1.00 | 24.81 | N |
| ATOM | 460 | CZ2 | TRP | | 73 | 4.438 | 69.592 | −34.978 | 1.00 | 24.97 | C |
| ATOM | 461 | CZ3 | TRP | | 73 | 4.638 | 67.444 | −36.084 | 1.00 | 28.24 | C |
| ATOM | 462 | CH2 | TRP | | 73 | 5.179 | 68.682 | −35.691 | 1.00 | 25.90 | C |
| ATOM | 463 | C | TRP | | 73 | 1.746 | 65.392 | −33.317 | 1.00 | 27.65 | C |
| ATOM | 464 | O | TRP | | 73 | 2.247 | 64.610 | −34.118 | 1.00 | 27.77 | O |
| ATOM | 465 | N | ARG | | 74 | 2.422 | 65.877 | −32.278 | 1.00 | 28.75 | N |
| ATOM | 466 | CA | ARG | | 74 | 3.814 | 65.494 | −32.047 | 1.00 | 29.62 | C |
| ATOM | 467 | CB | ARG | | 74 | 4.542 | 66.550 | −31.210 | 1.00 | 28.45 | C |
| ATOM | 468 | CG | ARG | | 74 | 4.584 | 67.929 | −31.836 | 1.00 | 29.77 | C |
| ATOM | 469 | CD | ARG | | 74 | 5.736 | 68.725 | −31.251 | 1.00 | 28.83 | C |
| ATOM | 470 | NE | ARG | | 74 | 7.003 | 68.086 | −31.582 | 1.00 | 28.53 | N |
| ATOM | 471 | CZ | ARG | | 74 | 7.992 | 67.881 | −30.720 | 1.00 | 30.40 | C |
| ATOM | 472 | NH1 | ARG | | 74 | 7.869 | 68.267 | −29.456 | 1.00 | 31.01 | N |
| ATOM | 473 | NH2 | ARG | | 74 | 9.105 | 67.284 | −31.128 | 1.00 | 30.12 | N |
| ATOM | 474 | C | ARG | | 74 | 3.946 | 64.142 | −31.359 | 1.00 | 30.21 | C |
| ATOM | 475 | O | ARG | | 74 | 4.894 | 63.402 | −31.616 | 1.00 | 30.04 | O |
| ATOM | 476 | N | GLN | | 75 | 3.016 | 63.837 | −30.462 | 1.00 | 30.70 | N |
| ATOM | 477 | CA | GLN | | 75 | 3.026 | 62.556 | −29.766 | 1.00 | 32.11 | C |
| ATOM | 478 | CB | GLN | | 75 | 4.120 | 62.508 | −28.691 | 1.00 | 34.24 | C |
| ATOM | 479 | CG | GLN | | 75 | 4.043 | 63.587 | −27.644 | 1.00 | 38.32 | C |
| ATOM | 480 | CD | GLN | | 75 | 5.032 | 63.366 | −26.511 | 1.00 | 40.81 | C |
| ATOM | 481 | OE1 | GLN | | 75 | 5.206 | 64.227 | −25.648 | 1.00 | 41.30 | O |
| ATOM | 482 | NE2 | GLN | | 75 | 5.679 | 62.205 | −26.504 | 1.00 | 41.57 | N |
| ATOM | 483 | C | GLN | | 75 | 1.657 | 62.280 | −29.159 | 1.00 | 31.47 | C |
| ATOM | 484 | O | GLN | | 75 | 1.046 | 63.145 | −28.527 | 1.00 | 29.42 | O |
| ATOM | 485 | N | GLU | | 76 | 1.185 | 61.059 | −29.375 | 1.00 | 31.28 | N |
| ATOM | 486 | CA | GLU | | 76 | −0.125 | 60.630 | −28.912 | 1.00 | 30.49 | C |
| ATOM | 487 | CB | GLU | | 76 | −0.372 | 59.195 | −29.381 | 1.00 | 30.39 | C |
| ATOM | 488 | CG | GLU | | 76 | −1.828 | 58.780 | −29.381 | 1.00 | 30.82 | C |
| ATOM | 489 | CD | GLU | | 76 | −2.078 | 57.547 | −30.233 | 1.00 | 31.41 | C |
| ATOM | 490 | OE1 | GLU | | 76 | −3.260 | 57.178 | −30.405 | 1.00 | 32.43 | O |
| ATOM | 491 | OE2 | GLU | | 76 | −1.099 | 56.953 | −30.732 | 1.00 | 28.34 | O |
| ATOM | 492 | C | GLU | | 76 | −0.331 | 60.738 | −27.405 | 1.00 | 31.28 | C |
| ATOM | 493 | O | GLU | | 76 | −1.391 | 61.170 | −26.948 | 1.00 | 29.48 | O |
| ATOM | 494 | N | LYS | | 77 | 0.684 | 60.354 | −26.637 | 1.00 | 32.54 | N |
| ATOM | 495 | CA | LYS | | 77 | 0.606 | 60.395 | −25.179 | 1.00 | 34.50 | C |
| ATOM | 496 | CB | LYS | | 77 | 0.798 | 58.979 | −24.620 | 1.00 | 35.38 | C |
| ATOM | 497 | CG | LYS | | 77 | 0.761 | 58.868 | −23.101 | 1.00 | 37.93 | C |
| ATOM | 498 | CD | LYS | | 77 | −0.646 | 59.047 | −22.551 | 1.00 | 39.29 | C |
| ATOM | 499 | CE | LYS | | 77 | −0.688 | 58.791 | −21.048 | 1.00 | 39.81 | C |
| ATOM | 500 | NZ | LYS | | 77 | −0.155 | 57.439 | −20.697 | 1.00 | 40.40 | N |
| ATOM | 501 | C | LYS | | 77 | 1.664 | 61.330 | −24.584 | 1.00 | 34.14 | C |
| ATOM | 502 | O | LYS | | 77 | 2.823 | 60.945 | −24.441 | 1.00 | 33.98 | O |
| ATOM | 503 | N | ILE | | 78 | 1.261 | 62.552 | −24.243 | 1.00 | 33.79 | N |
| ATOM | 504 | CA | ILE | | 78 | 2.176 | 63.532 | −23.651 | 1.00 | 34.37 | C |
| ATOM | 505 | CB | ILE | | 78 | 1.596 | 64.964 | −23.722 | 1.00 | 34.17 | C |
| ATOM | 506 | CG2 | ILE | | 78 | 2.588 | 65.956 | −23.106 | 1.00 | 33.11 | C |
| ATOM | 507 | CG1 | ILE | | 78 | 1.257 | 65.335 | −25.172 | 1.00 | 35.39 | C |
| ATOM | 508 | CD1 | ILE | | 78 | 2.433 | 65.768 | −26.010 | 1.00 | 35.38 | C |
| ATOM | 509 | C | ILE | | 78 | 2.363 | 63.176 | −22.175 | 1.00 | 34.21 | C |
| ATOM | 510 | O | ILE | | 78 | 1.407 | 63.220 | −21.402 | 1.00 | 33.74 | O |
| ATOM | 511 | N | THR | | 79 | 3.587 | 62.847 | −21.776 | 1.00 | 34.52 | N |
| ATOM | 512 | CA | THR | | 79 | 3.839 | 62.469 | −20.390 | 1.00 | 35.59 | C |
| ATOM | 513 | CB | THR | | 79 | 4.379 | 61.034 | −20.325 | 1.00 | 36.73 | C |
| ATOM | 514 | OG1 | THR | | 79 | 5.616 | 60.955 | −21.041 | 1.00 | 37.76 | O |
| ATOM | 515 | CG2 | THR | | 79 | 3.384 | 60.073 | −20.949 | 1.00 | 38.34 | C |
| ATOM | 516 | C | THR | | 79 | 4.780 | 63.378 | −19.588 | 1.00 | 35.57 | C |
| ATOM | 517 | O | THR | | 79 | 4.954 | 63.176 | −18.385 | 1.00 | 35.59 | O |
| ATOM | 518 | N | ARG | | 80 | 5.385 | 64.369 | −20.239 | 1.00 | 33.23 | N |
| ATOM | 519 | CA | ARG | | 80 | 6.296 | 65.279 | −19.541 | 1.00 | 30.67 | C |
| ATOM | 520 | CB | ARG | | 80 | 6.904 | 66.287 | −20.528 | 1.00 | 31.47 | C |
| ATOM | 521 | CG | ARG | | 80 | 5.882 | 67.267 | −21.098 | 1.00 | 34.41 | C |
| ATOM | 522 | CD | ARG | | 80 | 6.529 | 68.381 | −21.915 | 1.00 | 35.34 | C |
| ATOM | 523 | NE | ARG | | 80 | 6.316 | 68.205 | −23.349 | 1.00 | 39.31 | N |
| ATOM | 524 | CZ | ARG | | 80 | 7.139 | 67.541 | −24.148 | 1.00 | 38.61 | C |
| ATOM | 525 | NH1 | ARG | | 80 | 8.240 | 66.989 | −23.652 | 1.00 | 42.07 | N |
| ATOM | 526 | NH2 | ARG | | 80 | 6.863 | 67.425 | −25.440 | 1.00 | 36.80 | N |
| ATOM | 527 | C | ARG | | 80 | 5.557 | 66.039 | −18.439 | 1.00 | 28.66 | C |
| ATOM | 528 | O | ARG | | 80 | 4.371 | 66.341 | −18.570 | 1.00 | 28.06 | O |
| ATOM | 529 | N | THR | | 81 | 6.260 | 66.353 | −17.356 | 1.00 | 27.99 | N |
| ATOM | 530 | CA | THR | | 81 | 5.662 | 67.093 | −16.249 | 1.00 | 28.26 | C |
| ATOM | 531 | CB | THR | | 81 | 6.500 | 66.949 | −14.965 | 1.00 | 29.49 | C |
| ATOM | 532 | OG1 | THR | | 81 | 7.796 | 67.530 | −15.168 | 1.00 | 29.06 | O |
| ATOM | 533 | CG2 | THR | | 81 | 6.665 | 65.476 | −14.601 | 1.00 | 30.46 | C |
| ATOM | 534 | C | THR | | 81 | 5.587 | 68.575 | −16.605 | 1.00 | 28.36 | C |
| ATOM | 535 | O | THR | | 81 | 6.197 | 69.011 | −17.583 | 1.00 | 27.32 | O |
| ATOM | 536 | N | LYS | | 82 | 4.842 | 69.347 | −15.819 | 1.00 | 28.42 | N |
| ATOM | 537 | CA | LYS | | 82 | 4.735 | 70.782 | −16.069 | 1.00 | 30.70 | C |
| ATOM | 538 | CB | LYS | | 82 | 3.819 | 71.465 | −15.053 | 1.00 | 33.39 | C |
| ATOM | 539 | CG | LYS | | 82 | 2.406 | 70.926 | −14.977 | 1.00 | 40.16 | C |
| ATOM | 540 | CD | LYS | | 82 | 1.455 | 71.969 | −14.381 | 1.00 | 43.22 | C |
| ATOM | 541 | CE | LYS | | 82 | 1.969 | 72.541 | −13.060 | 1.00 | 44.12 | C |
| ATOM | 542 | NZ | LYS | | 82 | 1.095 | 73.646 | −12.571 | 1.00 | 43.54 | N |
| ATOM | 543 | C | LYS | | 82 | 6.119 | 71.411 | −15.950 | 1.00 | 29.51 | C |
| ATOM | 544 | O | LYS | | 82 | 6.466 | 72.318 | −16.703 | 1.00 | 27.50 | O |
| ATOM | 545 | N | GLU | | 83 | 6.902 | 70.926 | −14.990 | 1.00 | 28.04 | N |
| ATOM | 546 | CA | GLU | | 83 | 8.238 | 71.453 | −14.769 | 1.00 | 27.05 | C |
| ATOM | 547 | CB | GLU | | 83 | 8.832 | 70.851 | −13.492 | 1.00 | 30.17 | C |
| ATOM | 548 | CG | GLU | | 83 | 8.111 | 71.299 | −12.215 | 1.00 | 33.55 | C |
| ATOM | 549 | CD | GLU | | 83 | 6.737 | 70.652 | −12.033 | 1.00 | 36.81 | C |
| ATOM | 550 | OE1 | GLU | | 83 | 5.954 | 71.139 | −11.186 | 1.00 | 37.96 | O |
| ATOM | 551 | OE2 | GLU | | 83 | 6.445 | 69.652 | −12.723 | 1.00 | 37.01 | O |
| ATOM | 552 | C | GLU | | 83 | 9.166 | 71.225 | −15.960 | 1.00 | 25.77 | C |
| ATOM | 553 | O | GLU | | 83 | 9.948 | 72.107 | −16.312 | 1.00 | 24.44 | O |
| ATOM | 554 | N | GLU | | 84 | 9.081 | 70.052 | −16.583 | 1.00 | 25.03 | N |
| ATOM | 555 | CA | GLU | | 84 | 9.911 | 69.748 | −17.747 | 1.00 | 25.28 | C |
| ATOM | 556 | CB | GLU | | 84 | 9.796 | 68.271 | −18.122 | 1.00 | 27.06 | C |
| ATOM | 557 | CG | GLU | | 84 | 10.215 | 67.306 | −17.034 | 1.00 | 31.15 | C |
| ATOM | 558 | CD | GLU | | 84 | 10.035 | 65.862 | −17.453 | 1.00 | 32.30 | C |
| ATOM | 559 | OE1 | GLU | | 84 | 8.886 | 65.468 | −17.747 | 1.00 | 32.10 | O |
| ATOM | 560 | OE2 | GLU | | 84 | 11.041 | 65.125 | −17.495 | 1.00 | 34.89 | O |
| ATOM | 561 | C | GLU | | 84 | 9.435 | 70.600 | −18.928 | 1.00 | 25.79 | C |
| ATOM | 562 | O | GLU | | 84 | 10.232 | 71.041 | −19.763 | 1.00 | 24.81 | O |
| ATOM | 563 | N | ALA | | 85 | 8.128 | 70.818 | −18.994 | 1.00 | 22.77 | N |
| ATOM | 564 | CA | ALA | | 85 | 7.548 | 71.627 | −20.057 | 1.00 | 23.79 | C |
| ATOM | 565 | CB | ALA | | 85 | 6.021 | 71.610 | −19.959 | 1.00 | 24.47 | C |
| ATOM | 566 | C | ALA | | 85 | 8.065 | 73.061 | −19.947 | 1.00 | 22.66 | C |
| ATOM | 567 | O | ALA | | 85 | 8.398 | 73.691 | −20.956 | 1.00 | 20.48 | O |
| ATOM | 568 | N | LEU | | 86 | 8.132 | 73.577 | −18.721 | 1.00 | 21.24 | N |
| ATOM | 569 | CA | LEU | | 86 | 8.613 | 74.940 | −18.522 | 1.00 | 21.45 | C |
| ATOM | 570 | CB | LEU | | 86 | 8.435 | 75.379 | −17.065 | 1.00 | 20.41 | C |
| ATOM | 571 | CG | LEU | | 86 | 8.900 | 76.812 | −16.745 | 1.00 | 22.39 | C |
| ATOM | 572 | CD1 | LEU | | 86 | 8.232 | 77.800 | −17.706 | 1.00 | 19.64 | C |
| ATOM | 573 | CD2 | LEU | | 86 | 8.549 | 77.165 | −15.302 | 1.00 | 21.24 | C |
| ATOM | 574 | C | LEU | | 86 | 10.082 | 75.056 | −18.923 | 1.00 | 22.20 | C |
| ATOM | 575 | O | LEU | | 86 | 10.502 | 76.087 | −19.451 | 1.00 | 21.40 | O |
| ATOM | 576 | N | GLU | | 87 | 10.864 | 74.008 | −18.677 | 1.00 | 22.64 | N |
| ATOM | 577 | CA | GLU | | 87 | 12.275 | 74.051 | −19.047 | 1.00 | 25.14 | C |
| ATOM | 578 | CB | GLU | | 87 | 13.045 | 72.872 | −18.446 | 1.00 | 27.52 | C |
| ATOM | 579 | CG | GLU | | 87 | 13.351 | 73.092 | −16.974 | 1.00 | 36.19 | C |
| ATOM | 580 | CD | GLU | | 87 | 13.825 | 74.520 | −16.705 | 1.00 | 40.59 | C |
| ATOM | 581 | OE1 | GLU | | 87 | 14.907 | 74.901 | −17.208 | 1.00 | 41.45 | O |
| ATOM | 582 | OE2 | GLU | | 87 | 13.105 | 75.266 | −16.001 | 1.00 | 43.32 | O |
| ATOM | 583 | C | GLU | | 87 | 12.456 | 74.081 | −20.550 | 1.00 | 24.48 | C |
| ATOM | 584 | O | GLU | | 87 | 13.384 | 74.719 | −21.056 | 1.00 | 23.19 | O |
| ATOM | 585 | N | LEU | | 88 | 11.576 | 73.383 | −21.263 | 1.00 | 21.62 | N |
| ATOM | 586 | CA | LEU | | 88 | 11.626 | 73.358 | −22.717 | 1.00 | 20.50 | C |
| ATOM | 587 | CB | LEU | | 88 | 10.607 | 72.355 | −23.266 | 1.00 | 20.97 | C |
| ATOM | 588 | CG | LEU | | 88 | 11.000 | 70.885 | −23.100 | 1.00 | 23.42 | C |
| ATOM | 589 | CD1 | LEU | | 88 | 9.818 | 69.973 | −23.410 | 1.00 | 22.75 | C |
| ATOM | 590 | CD2 | LEU | | 88 | 12.178 | 70.584 | −24.015 | 1.00 | 23.67 | C |
| ATOM | 591 | C | LEU | | 88 | 11.302 | 74.759 | −23.220 | 1.00 | 20.27 | C |
| ATOM | 592 | O | LEU | | 88 | 12.004 | 75.303 | −24.084 | 1.00 | 19.12 | O |
| ATOM | 593 | N | ILE | | 89 | 10.235 | 75.338 | −22.670 | 1.00 | 18.57 | N |
| ATOM | 594 | CA | ILE | | 89 | 9.807 | 76.680 | −23.034 | 1.00 | 18.06 | C |
| ATOM | 595 | CB | ILE | | 89 | 8.563 | 77.104 | −22.213 | 1.00 | 18.18 | C |
| ATOM | 596 | CG2 | ILE | | 89 | 8.399 | 78.612 | −22.244 | 1.00 | 15.52 | C |
| ATOM | 597 | CG1 | ILE | | 89 | 7.307 | 76.412 | −22.755 | 1.00 | 19.04 | C |
| ATOM | 598 | CD1 | ILE | | 89 | 6.773 | 76.998 | −24.070 | 1.00 | 20.49 | C |
| ATOM | 599 | C | ILE | | 89 | 10.938 | 77.689 | −22.786 | 1.00 | 18.48 | C |
| ATOM | 600 | O | ILE | | 89 | 11.253 | 78.509 | −23.655 | 1.00 | 17.54 | O |
| ATOM | 601 | N | ASN | | 90 | 11.543 | 77.635 | −21.601 | 1.00 | 17.17 | N |
| ATOM | 602 | CA | ASN | | 90 | 12.626 | 78.562 | −21.279 | 1.00 | 17.98 | C |
| ATOM | 603 | CB | ASN | | 90 | 13.153 | 78.321 | −19.862 | 1.00 | 19.03 | C |
| ATOM | 604 | CG | ASN | | 90 | 12.175 | 78.766 | −18.792 | 1.00 | 21.48 | C |
| ATOM | 605 | OD1 | ASN | | 90 | 11.453 | 79.751 | −18.963 | 1.00 | 24.53 | O |
| ATOM | 606 | ND2 | ASN | | 90 | 12.163 | 78.055 | −17.670 | 1.00 | 23.45 | N |
| ATOM | 607 | C | ASN | | 90 | 13.767 | 78.432 | −22.277 | 1.00 | 17.36 | C |
| ATOM | 608 | O | ASN | | 90 | 14.382 | 79.428 | −22.652 | 1.00 | 17.80 | O |
| ATOM | 609 | N | GLY | | 91 | 14.035 | 77.203 | −22.712 | 1.00 | 16.95 | N |
| ATOM | 610 | CA | GLY | | 91 | 15.096 | 76.966 | −23.676 | 1.00 | 17.04 | C |
| ATOM | 611 | C | GLY | | 91 | 14.778 | 77.566 | −25.037 | 1.00 | 17.94 | C |
| ATOM | 612 | O | GLY | | 91 | 15.658 | 78.119 | −25.705 | 1.00 | 17.19 | O |
| ATOM | 613 | N | TYR | | 92 | 13.523 | 77.456 | −25.465 | 1.00 | 16.53 | N |
| ATOM | 614 | CA | TYR | | 92 | 13.122 | 78.014 | −26.751 | 1.00 | 16.32 | C |
| ATOM | 615 | CB | TYR | | 92 | 11.684 | 77.609 | −27.094 | 1.00 | 17.49 | C |
| ATOM | 616 | CG | TYR | | 92 | 11.478 | 76.126 | −27.298 | 1.00 | 18.42 | C |
| ATOM | 617 | CD1 | TYR | | 92 | 10.278 | 75.518 | −26.928 | 1.00 | 19.25 | C |
| ATOM | 618 | CE1 | TYR | | 92 | 10.078 | 74.147 | −27.108 | 1.00 | 21.14 | C |
| ATOM | 619 | CD2 | TYR | | 92 | 12.474 | 75.330 | −27.861 | 1.00 | 19.07 | C |
| ATOM | 620 | CE2 | TYR | | 92 | 12.283 | 73.960 | −28.045 | 1.00 | 20.77 | C |
| ATOM | 621 | CZ | TYR | | 92 | 11.082 | 73.380 | −27.665 | 1.00 | 21.45 | C |
| ATOM | 622 | OH | TYR | | 92 | 10.886 | 72.029 | −27.844 | 1.00 | 22.71 | O |
| ATOM | 623 | C | TYR | | 92 | 13.216 | 79.535 | −26.695 | 1.00 | 14.77 | C |
| ATOM | 624 | O | TYR | | 92 | 13.644 | 80.168 | −27.658 | 1.00 | 16.96 | O |
| ATOM | 625 | N | ILE | | 93 | 12.809 | 80.114 | −25.568 | 1.00 | 14.47 | N |
| ATOM | 626 | CA | ILE | | 93 | 12.863 | 81.562 | −25.405 | 1.00 | 15.25 | C |
| ATOM | 627 | CB | ILE | | 93 | 12.247 | 82.006 | −24.047 | 1.00 | 14.64 | C |
| ATOM | 628 | CG2 | ILE | | 93 | 12.527 | 83.496 | −23.788 | 1.00 | 15.01 | C |
| ATOM | 629 | CG1 | ILE | | 93 | 10.729 | 81.796 | −24.079 | 1.00 | 16.02 | C |
| ATOM | 630 | CD1 | ILE | | 93 | 10.044 | 82.061 | −22.748 | 1.00 | 18.29 | C |
| ATOM | 631 | C | ILE | | 93 | 14.317 | 82.030 | −25.505 | 1.00 | 15.51 | C |
| ATOM | 632 | O | ILE | | 93 | 14.607 | 83.052 | −26.132 | 1.00 | 13.31 | O |
| ATOM | 633 | N | GLN | | 94 | 15.235 | 81.275 | −24.912 | 1.00 | 15.38 | N |
| ATOM | 634 | CA | GLN | | 94 | 16.638 | 81.661 | −24.985 | 1.00 | 18.01 | C |
| ATOM | 635 | CB | GLN | | 94 | 17.501 | 80.744 | −24.115 | 1.00 | 19.58 | C |
| ATOM | 636 | CG | GLN | | 94 | 17.278 | 80.938 | −22.623 | 1.00 | 23.64 | C |
| ATOM | 637 | CD | GLN | | 94 | 18.292 | 80.188 | −21.783 | 1.00 | 28.98 | C |
| ATOM | 638 | OE1 | GLN | | 94 | 18.396 | 78.963 | −21.861 | 1.00 | 31.96 | O |
| ATOM | 639 | NE2 | GLN | | 94 | 19.052 | 80.923 | −20.975 | 1.00 | 32.15 | N |
| ATOM | 640 | C | GLN | | 94 | 17.153 | 81.653 | −26.424 | 1.00 | 18.29 | C |
| ATOM | 641 | O | GLN | | 94 | 17.815 | 82.600 | −26.853 | 1.00 | 19.01 | O |
| ATOM | 642 | N | LYS | | 95 | 16.841 | 80.598 | −27.172 | 1.00 | 18.45 | N |
| ATOM | 643 | CA | LYS | | 95 | 17.299 | 80.503 | −28.551 | 1.00 | 19.26 | C |
| ATOM | 644 | CB | LYS | | 95 | 17.004 | 79.110 | −29.123 | 1.00 | 23.20 | C |
| ATOM | 645 | CG | LYS | | 95 | 17.671 | 77.964 | −28.369 | 1.00 | 28.97 | C |
| ATOM | 646 | CD | LYS | | 95 | 17.534 | 76.647 | −29.138 | 1.00 | 31.86 | C |
| ATOM | 647 | CE | LYS | | 95 | 17.704 | 75.420 | −28.231 | 1.00 | 36.09 | C |
| ATOM | 648 | NZ | LYS | | 95 | 16.525 | 75.174 | −27.315 | 1.00 | 33.67 | N |
| ATOM | 649 | C | LYS | | 95 | 16.642 | 81.569 | −29.421 | 1.00 | 18.07 | C |
| ATOM | 650 | O | LYS | | 95 | 17.231 | 82.049 | −30.393 | 1.00 | 18.02 | O |
| ATOM | 651 | N | ILE | | 96 | 15.416 | 81.940 | −29.077 | 1.00 | 15.70 | N |
| ATOM | 652 | CA | ILE | | 96 | 14.715 | 82.959 | −29.842 | 1.00 | 16.11 | C |
| ATOM | 653 | CB | ILE | | 96 | 13.207 | 82.991 | −29.478 | 1.00 | 16.36 | C |
| ATOM | 654 | CG2 | ILE | | 96 | 12.548 | 84.239 | −30.062 | 1.00 | 15.55 | C |
| ATOM | 655 | CG1 | ILE | | 96 | 12.530 | 81.718 | −30.004 | 1.00 | 16.10 | C |
| ATOM | 656 | CD1 | ILE | | 96 | 11.063 | 81.562 | −29.591 | 1.00 | 21.90 | C |
| ATOM | 657 | C | ILE | | 96 | 15.345 | 84.323 | −29.572 | 1.00 | 15.27 | C |
| ATOM | 658 | O | ILE | | 96 | 15.622 | 85.087 | −30.497 | 1.00 | 16.10 | O |
| ATOM | 659 | N | LYS | | 97 | 15.591 | 84.620 | −28.303 | 1.00 | 14.87 | N |
| ATOM | 660 | CA | LYS | | 97 | 16.177 | 85.904 | −27.946 | 1.00 | 16.12 | C |
| ATOM | 661 | CB | LYS | | 97 | 16.095 | 86.140 | −26.438 | 1.00 | 16.60 | C |
| ATOM | 662 | CG | LYS | | 97 | 14.713 | 86.525 | −25.940 | 1.00 | 18.03 | C |
| ATOM | 663 | CD | LYS | | 97 | 14.804 | 87.047 | −24.522 | 1.00 | 19.06 | C |
| ATOM | 664 | CE | LYS | | 97 | 13.481 | 87.604 | −24.048 | 1.00 | 20.51 | C |
| ATOM | 665 | NZ | LYS | | 97 | 13.591 | 88.152 | −22.668 | 1.00 | 18.92 | N |
| ATOM | 666 | C | LYS | | 97 | 17.620 | 86.051 | −28.402 | 1.00 | 15.43 | C |
| ATOM | 667 | O | LYS | | 97 | 18.071 | 87.165 | −28.657 | 1.00 | 16.13 | O |
| ATOM | 668 | N | SER | | 98 | 18.335 | 84.937 | −28.508 | 1.00 | 16.35 | N |
| ATOM | 669 | CA | SER | | 98 | 19.733 | 84.970 | −28.945 | 1.00 | 18.06 | C |
| ATOM | 670 | CB | SER | | 98 | 20.474 | 83.713 | −28.472 | 1.00 | 18.53 | C |
| ATOM | 671 | OG | SER | | 98 | 20.024 | 82.551 | −29.154 | 1.00 | 18.00 | O |
| ATOM | 672 | C | SER | | 98 | 19.832 | 85.070 | −30.462 | 1.00 | 19.16 | C |
| ATOM | 673 | O | SER | | 98 | 20.876 | 85.433 | −31.012 | 1.00 | 19.15 | O |
| ATOM | 674 | N | GLY | | 99 | 18.738 | 84.750 | −31.144 | 1.00 | 18.86 | N |
| ATOM | 675 | CA | GLY | | 99 | 18.757 | 84.802 | −32.589 | 1.00 | 20.20 | C |
| ATOM | 676 | C | GLY | | 99 | 19.252 | 83.490 | −33.162 | 1.00 | 21.35 | C |
| ATOM | 677 | O | GLY | | 99 | 19.389 | 83.353 | −34.375 | 1.00 | 23.18 | O |
| ATOM | 678 | N | GLU | | 100 | 19.530 | 82.521 | −32.293 | 1.00 | 23.66 | N |
| ATOM | 679 | CA | GLU | | 100 | 19.996 | 81.213 | −32.742 | 1.00 | 26.39 | C |
| ATOM | 680 | CB | GLU | | 100 | 20.320 | 80.321 | −31.538 | 1.00 | 28.99 | C |
| ATOM | 681 | CG | GLU | | 100 | 21.631 | 80.675 | −30.845 | 1.00 | 37.69 | C |
| ATOM | 682 | CD | GLU | | 100 | 21.837 | 79.928 | −29.537 | 1.00 | 41.15 | C |
| ATOM | 683 | OE1 | GLU | | 100 | 21.211 | 80.305 | −28.521 | 1.00 | 41.91 | O |
| ATOM | 684 | OE2 | GLU | | 100 | 22.625 | 78.957 | −29.528 | 1.00 | 45.14 | O |
| ATOM | 685 | C | GLU | | 100 | 18.913 | 80.562 | −33.595 | 1.00 | 26.46 | C |
| ATOM | 686 | O | GLU | | 100 | 19.192 | 79.974 | −34.635 | 1.00 | 25.99 | O |
| ATOM | 687 | N | GLU | | 101 | 17.670 | 80.693 | −33.144 | 1.00 | 26.61 | N |
| ATOM | 688 | CA | GLU | | 101 | 16.511 | 80.132 | −33.829 | 1.00 | 29.10 | C |
| ATOM | 689 | CB | GLU | | 101 | 16.009 | 78.900 | −33.071 | 1.00 | 32.16 | C |
| ATOM | 690 | CG | GLU | | 101 | 16.807 | 77.641 | −33.322 | 1.00 | 37.54 | C |
| ATOM | 691 | CD | GLU | | 101 | 16.420 | 76.982 | −34.627 | 1.00 | 39.94 | C |
| ATOM | 692 | OE1 | GLU | | 101 | 16.469 | 77.660 | −35.678 | 1.00 | 43.41 | O |
| ATOM | 693 | OE2 | GLU | | 101 | 16.059 | 75.788 | −34.604 | 1.00 | 42.28 | O |
| ATOM | 694 | C | GLU | | 101 | 15.411 | 81.182 | −33.842 | 1.00 | 28.10 | C |
| ATOM | 695 | O | GLU | | 101 | 15.305 | 81.965 | −32.902 | 1.00 | 29.30 | O |
| ATOM | 696 | N | ASP | | 102 | 14.602 | 81.232 | −34.894 | 1.00 | 25.39 | N |
| ATOM | 697 | CA | ASP | | 102 | 13.526 | 82.205 | −34.865 | 1.00 | 26.40 | C |
| ATOM | 698 | CB | ASP | | 102 | 13.366 | 82.937 | −36.207 | 1.00 | 32.21 | C |
| ATOM | 699 | CG | ASP | | 102 | 13.278 | 82.010 | −37.387 | 1.00 | 36.42 | C |
| ATOM | 700 | OD1 | ASP | | 102 | 13.566 | 82.486 | −38.513 | 1.00 | 37.45 | O |
| ATOM | 701 | OD2 | ASP | | 102 | 12.915 | 80.828 | −37.201 | 1.00 | 39.49 | O |
| ATOM | 702 | C | ASP | | 102 | 12.235 | 81.532 | −34.413 | 1.00 | 24.14 | C |
| ATOM | 703 | O | ASP | | 102 | 12.075 | 80.311 | −34.517 | 1.00 | 22.07 | O |
| ATOM | 704 | N | PHE | | 103 | 11.338 | 82.337 | −33.865 | 1.00 | 21.65 | N |
| ATOM | 705 | CA | PHE | | 103 | 10.069 | 81.849 | −33.344 | 1.00 | 22.12 | C |
| ATOM | 706 | CB | PHE | | 103 | 9.158 | 83.036 | −33.019 | 1.00 | 22.80 | C |
| ATOM | 707 | CG | PHE | | 103 | 7.826 | 82.635 | −32.463 | 1.00 | 25.46 | C |
| ATOM | 708 | CD1 | PHE | | 103 | 7.652 | 82.464 | −31.094 | 1.00 | 25.26 | C |
| ATOM | 709 | CD2 | PHE | | 103 | 6.758 | 82.363 | −33.317 | 1.00 | 26.44 | C |
| ATOM | 710 | CE1 | PHE | | 103 | 6.433 | 82.023 | −30.582 | 1.00 | 27.70 | C |
| ATOM | 711 | CE2 | PHE | | 103 | 5.537 | 81.920 | −32.814 | 1.00 | 26.22 | C |
| ATOM | 712 | CZ | PHE | | 103 | 5.376 | 81.750 | −31.445 | 1.00 | 26.33 | C |
| ATOM | 713 | C | PHE | | 103 | 9.324 | 80.891 | −34.277 | 1.00 | 20.98 | C |
| ATOM | 714 | O | PHE | | 103 | 8.910 | 79.804 | −33.868 | 1.00 | 18.46 | O |
| ATOM | 715 | N | GLU | | 104 | 9.154 | 81.310 | −35.526 | 1.00 | 20.78 | N |
| ATOM | 716 | CA | GLU | | 104 | 8.421 | 80.528 | −36.517 | 1.00 | 22.06 | C |
| ATOM | 717 | CB | GLU | | 104 | 8.401 | 81.277 | −37.857 | 1.00 | 22.53 | C |
| ATOM | 718 | CG | GLU | | 104 | 7.705 | 82.628 | −37.781 | 1.00 | 22.38 | C |
| ATOM | 719 | CD | GLU | | 104 | 8.637 | 83.761 | −37.370 | 1.00 | 23.45 | C |
| ATOM | 720 | OE1 | GLU | | 104 | 9.647 | 83.505 | −36.672 | 1.00 | 19.39 | O |
| ATOM | 721 | OE2 | GLU | | 104 | 8.344 | 84.916 | −37.744 | 1.00 | 25.23 | O |
| ATOM | 722 | C | GLU | | 104 | 8.957 | 79.120 | −36.718 | 1.00 | 21.94 | C |
| ATOM | 723 | O | GLU | | 104 | 8.186 | 78.163 | −36.853 | 1.00 | 22.04 | O |
| ATOM | 724 | N | SER | | 105 | 10.277 | 78.997 | −36.742 | 1.00 | 20.26 | N |
| ATOM | 725 | CA | SER | | 105 | 10.917 | 77.704 | −36.932 | 1.00 | 21.32 | C |
| ATOM | 726 | CB | SER | | 105 | 12.415 | 77.890 | −37.141 | 1.00 | 21.99 | C |
| ATOM | 727 | OG | SER | | 105 | 13.076 | 76.644 | −37.147 | 1.00 | 26.14 | O |
| ATOM | 728 | C | SER | | 105 | 10.670 | 76.765 | −35.753 | 1.00 | 19.93 | C |
| ATOM | 729 | O | SER | | 105 | 10.359 | 75.590 | −35.950 | 1.00 | 19.78 | O |
| ATOM | 730 | N | LEU | | 106 | 10.806 | 77.278 | −34.533 | 1.00 | 18.63 | N |
| ATOM | 731 | CA | LEU | | 106 | 10.588 | 76.458 | −33.337 | 1.00 | 18.86 | C |
| ATOM | 732 | CB | LEU | | 106 | 11.073 | 77.197 | −32.086 | 1.00 | 16.64 | C |
| ATOM | 733 | CG | LEU | | 106 | 12.577 | 77.498 | −32.039 | 1.00 | 17.92 | C |
| ATOM | 734 | CD1 | LEU | | 106 | 12.919 | 78.223 | −30.733 | 1.00 | 15.84 | C |
| ATOM | 735 | CD2 | LEU | | 106 | 13.360 | 76.196 | −32.149 | 1.00 | 16.67 | C |
| ATOM | 736 | C | LEU | | 106 | 9.119 | 76.069 | −33.166 | 1.00 | 18.22 | C |
| ATOM | 737 | O | LEU | | 106 | 8.807 | 74.962 | −32.715 | 1.00 | 18.69 | O |
| ATOM | 738 | N | ALA | | 107 | 8.217 | 76.982 | −33.511 | 1.00 | 18.46 | N |
| ATOM | 739 | CA | ALA | | 107 | 6.793 | 76.702 | −33.405 | 1.00 | 18.77 | C |
| ATOM | 740 | CB | ALA | | 107 | 5.974 | 77.956 | −33.710 | 1.00 | 15.50 | C |
| ATOM | 741 | C | ALA | | 107 | 6.427 | 75.588 | −34.387 | 1.00 | 19.40 | C |
| ATOM | 742 | O | ALA | | 107 | 5.729 | 74.636 | −34.024 | 1.00 | 19.91 | O |
| ATOM | 743 | N | SER | | 108 | 6.898 | 75.708 | −35.628 | 1.00 | 18.82 | N |
| ATOM | 744 | CA | SER | | 108 | 6.606 | 74.706 | −36.652 | 1.00 | 19.26 | C |
| ATOM | 745 | CB | SER | | 108 | 7.321 | 75.051 | −37.963 | 1.00 | 18.70 | C |
| ATOM | 746 | OG | SER | | 108 | 6.846 | 76.270 | −38.504 | 1.00 | 25.22 | O |
| ATOM | 747 | C | SER | | 108 | 7.070 | 73.331 | −36.201 | 1.00 | 20.29 | C |
| ATOM | 748 | O | SER | | 108 | 6.397 | 72.318 | −36.414 | 1.00 | 19.94 | O |
| ATOM | 749 | N | GLN | | 109 | 8.233 | 73.313 | −35.569 | 1.00 | 20.19 | N |
| ATOM | 750 | CA | GLN | | 109 | 8.840 | 72.083 | −35.111 | 1.00 | 22.48 | C |
| ATOM | 751 | CB | GLN | | 109 | 10.351 | 72.277 | −35.004 | 1.00 | 23.64 | C |
| ATOM | 752 | CG | GLN | | 109 | 11.091 | 72.370 | −36.318 | 1.00 | 23.93 | C |
| ATOM | 753 | CD | GLN | | 109 | 12.550 | 72.716 | −36.108 | 1.00 | 26.60 | C |
| ATOM | 754 | OE1 | GLN | | 109 | 12.922 | 73.892 | −36.027 | 1.00 | 28.06 | O |
| ATOM | 755 | NE2 | GLN | | 109 | 13.382 | 71.696 | −35.993 | 1.00 | 22.73 | N |
| ATOM | 756 | C | GLN | | 109 | 8.355 | 71.528 | −33.787 | 1.00 | 21.26 | C |
| ATOM | 757 | O | GLN | | 109 | 8.162 | 70.325 | −33.652 | 1.00 | 23.73 | O |
| ATOM | 758 | N | PHE | | 110 | 8.141 | 72.399 | −32.811 | 1.00 | 21.48 | N |
| ATOM | 759 | CA | PHE | | 110 | 7.799 | 71.919 | −31.489 | 1.00 | 21.05 | C |
| ATOM | 760 | CB | PHE | | 110 | 8.970 | 72.267 | −30.558 | 1.00 | 19.79 | C |
| ATOM | 761 | CG | PHE | | 110 | 10.314 | 71.871 | −31.111 | 1.00 | 21.19 | C |
| ATOM | 762 | CD1 | PHE | | 110 | 11.269 | 72.834 | −31.426 | 1.00 | 22.70 | C |
| ATOM | 763 | CD2 | PHE | | 110 | 10.618 | 70.532 | −31.339 | 1.00 | 21.12 | C |
| ATOM | 764 | CE1 | PHE | | 110 | 12.506 | 72.469 | −31.958 | 1.00 | 23.05 | C |
| ATOM | 765 | CE2 | PHE | | 110 | 11.850 | 70.156 | −31.872 | 1.00 | 23.03 | C |
| ATOM | 766 | CZ | PHE | | 110 | 12.798 | 71.125 | −32.183 | 1.00 | 22.97 | C |
| ATOM | 767 | C | PHE | | 110 | 6.485 | 72.286 | −30.816 | 1.00 | 20.97 | C |
| ATOM | 768 | O | PHE | | 110 | 6.225 | 71.818 | −29.709 | 1.00 | 23.48 | O |
| ATOM | 769 | N | SER | | 111 | 5.647 | 73.100 | −31.442 | 1.00 | 19.06 | N |
| ATOM | 770 | CA | SER | | 111 | 4.388 | 73.447 | −30.797 | 1.00 | 19.77 | C |
| ATOM | 771 | CB | SER | | 111 | 3.718 | 74.615 | −31.506 | 1.00 | 18.27 | C |
| ATOM | 772 | OG | SER | | 111 | 2.547 | 75.017 | −30.810 | 1.00 | 21.31 | O |
| ATOM | 773 | C | SER | | 111 | 3.438 | 72.245 | −30.800 | 1.00 | 20.72 | C |
| ATOM | 774 | O | SER | | 111 | 3.244 | 71.603 | −31.834 | 1.00 | 20.55 | O |
| ATOM | 775 | N | ASP | | 112 | 2.856 | 71.946 | −29.640 | 1.00 | 21.42 | N |
| ATOM | 776 | CA | ASP | | 112 | 1.925 | 70.820 | −29.503 | 1.00 | 22.28 | C |
| ATOM | 777 | CB | ASP | | 112 | 1.935 | 70.279 | −28.066 | 1.00 | 21.16 | C |
| ATOM | 778 | CG | ASP | | 112 | 3.157 | 69.433 | −27.758 | 1.00 | 20.93 | C |
| ATOM | 779 | OD1 | ASP | | 112 | 3.440 | 68.479 | −28.514 | 1.00 | 20.66 | O |
| ATOM | 780 | OD2 | ASP | | 112 | 3.831 | 69.708 | −26.740 | 1.00 | 21.10 | O |
| ATOM | 781 | C | ASP | | 112 | 0.506 | 71.256 | −29.855 | 1.00 | 22.61 | C |
| ATOM | 782 | O | ASP | | 112 | −0.439 | 70.955 | −29.127 | 1.00 | 23.97 | O |
| ATOM | 783 | N | CYS | | 113 | 0.363 | 71.966 | −30.970 | 1.00 | 23.07 | N |
| ATOM | 784 | CA | CYS | | 113 | −0.934 | 72.460 | −31.420 | 1.00 | 23.29 | C |
| ATOM | 785 | CB | CYS | | 113 | −1.135 | 73.896 | −30.913 | 1.00 | 23.08 | C |
| ATOM | 786 | SG | CYS | | 113 | −2.793 | 74.601 | −31.067 | 1.00 | 28.34 | S |
| ATOM | 787 | C | CYS | | 113 | −0.903 | 72.420 | −32.947 | 1.00 | 23.10 | C |
| ATOM | 788 | O | CYS | | 113 | 0.153 | 72.614 | −33.548 | 1.00 | 23.62 | O |
| ATOM | 789 | N | SER | | 114 | −2.043 | 72.161 | −33.578 | 1.00 | 21.44 | N |
| ATOM | 790 | CA | SER | | 114 | −2.080 | 72.095 | −35.038 | 1.00 | 22.98 | C |
| ATOM | 791 | CB | SER | | 114 | −3.482 | 71.701 | −35.532 | 1.00 | 24.83 | C |
| ATOM | 792 | OG | SER | | 114 | −4.475 | 72.609 | −35.085 | 1.00 | 30.07 | O |
| ATOM | 793 | C | SER | | 114 | −1.635 | 73.395 | −35.716 | 1.00 | 21.59 | C |
| ATOM | 794 | O | SER | | 114 | −1.246 | 73.380 | −36.887 | 1.00 | 20.64 | O |
| ATOM | 795 | N | SER | | 115 | −1.692 | 74.513 | −34.991 | 1.00 | 20.09 | N |
| ATOM | 796 | CA | SER | | 115 | −1.267 | 75.798 | −35.552 | 1.00 | 20.40 | C |
| ATOM | 797 | CB | SER | | 115 | −1.567 | 76.946 | −34.578 | 1.00 | 20.34 | C |
| ATOM | 798 | OG | SER | | 115 | −1.058 | 76.686 | −33.279 | 1.00 | 18.84 | O |
| ATOM | 799 | C | SER | | 115 | 0.224 | 75.782 | −35.897 | 1.00 | 19.24 | C |
| ATOM | 800 | O | SER | | 115 | 0.732 | 76.691 | −36.553 | 1.00 | 18.61 | O |
| ATOM | 801 | N | ALA | | 116 | 0.927 | 74.749 | −35.451 | 1.00 | 18.34 | N |
| ATOM | 802 | CA | ALA | | 116 | 2.347 | 74.626 | −35.751 | 1.00 | 17.43 | C |
| ATOM | 803 | CB | ALA | | 116 | 2.902 | 73.333 | −35.135 | 1.00 | 19.05 | C |
| ATOM | 804 | C | ALA | | 116 | 2.546 | 74.609 | −37.279 | 1.00 | 20.21 | C |
| ATOM | 805 | O | ALA | | 116 | 3.560 | 75.097 | −37.794 | 1.00 | 16.65 | O |
| ATOM | 806 | N | LYS | | 117 | 1.573 | 74.044 | −37.997 | 1.00 | 17.33 | N |
| ATOM | 807 | CA | LYS | | 117 | 1.660 | 73.944 | −39.454 | 1.00 | 18.42 | C |
| ATOM | 808 | CB | LYS | | 117 | 0.540 | 73.050 | −39.989 | 1.00 | 21.08 | C |
| ATOM | 809 | CG | LYS | | 117 | 0.574 | 71.646 | −39.421 | 1.00 | 23.48 | C |
| ATOM | 810 | CD | LYS | | 117 | 1.818 | 70.906 | −39.855 | 1.00 | 27.93 | C |
| ATOM | 811 | CE | LYS | | 117 | 1.901 | 69.528 | −39.196 | 1.00 | 30.51 | C |
| ATOM | 812 | NZ | LYS | | 117 | 2.304 | 69.595 | −37.766 | 1.00 | 28.97 | N |
| ATOM | 813 | C | LYS | | 117 | 1.619 | 75.303 | −40.141 | 1.00 | 17.85 | C |
| ATOM | 814 | O | LYS | | 117 | 2.006 | 75.423 | −41.304 | 1.00 | 15.46 | O |
| ATOM | 815 | N | ALA | | 118 | 1.139 | 76.312 | −39.417 | 1.00 | 16.31 | N |
| ATOM | 816 | CA | ALA | | 118 | 1.085 | 77.682 | −39.921 | 1.00 | 19.92 | C |
| ATOM | 817 | CB | ALA | | 118 | −0.332 | 78.255 | −39.773 | 1.00 | 18.29 | C |
| ATOM | 818 | C | ALA | | 118 | 2.086 | 78.532 | −39.120 | 1.00 | 20.46 | C |
| ATOM | 819 | O | ALA | | 118 | 1.835 | 79.708 | −38.834 | 1.00 | 19.19 | O |
| ATOM | 820 | N | ARG | | 119 | 3.208 | 77.920 | −38.746 | 1.00 | 19.21 | N |
| ATOM | 821 | CA | ARG | | 119 | 4.254 | 78.605 | −37.989 | 1.00 | 21.33 | C |
| ATOM | 822 | CB | ARG | | 119 | 4.891 | 79.712 | −38.846 | 1.00 | 23.33 | C |
| ATOM | 823 | CG | ARG | | 119 | 5.934 | 79.218 | −39.842 | 1.00 | 26.77 | C |
| ATOM | 824 | CD | ARG | | 119 | 6.122 | 80.180 | −41.018 | 1.00 | 31.98 | C |
| ATOM | 825 | NE | ARG | | 119 | 5.060 | 79.982 | −42.002 | 1.00 | 39.55 | N |
| ATOM | 826 | CZ | ARG | | 119 | 3.806 | 80.413 | −41.867 | 1.00 | 40.57 | C |
| ATOM | 827 | NH1 | ARG | | 119 | 3.437 | 81.096 | −40.790 | 1.00 | 42.45 | N |
| ATOM | 828 | NH2 | ARG | | 119 | 2.902 | 80.111 | −42.790 | 1.00 | 36.83 | N |
| ATOM | 829 | C | ARG | | 119 | 3.722 | 79.198 | −36.688 | 1.00 | 20.81 | C |
| ATOM | 830 | O | ARG | | 119 | 4.197 | 80.239 | −36.230 | 1.00 | 19.48 | O |
| ATOM | 831 | N | GLY | | 120 | 2.730 | 78.536 | −36.103 | 1.00 | 18.20 | N |
| ATOM | 832 | CA | GLY | | 120 | 2.163 | 79.009 | −34.853 | 1.00 | 20.13 | C |
| ATOM | 833 | C | GLY | | 120 | 1.083 | 80.065 | −34.973 | 1.00 | 20.28 | C |
| ATOM | 834 | O | GLY | | 120 | 0.455 | 80.412 | −33.971 | 1.00 | 19.14 | O |
| ATOM | 835 | N | ASP | | 121 | 0.850 | 80.570 | −36.186 | 1.00 | 20.68 | N |
| ATOM | 836 | CA | ASP | | 121 | −0.164 | 81.607 | −36.407 | 1.00 | 20.44 | C |
| ATOM | 837 | CB | ASP | | 121 | −0.150 | 82.082 | −37.867 | 1.00 | 20.79 | C |
| ATOM | 838 | CG | ASP | | 121 | −1.209 | 83.152 | −38.142 | 1.00 | 22.29 | C |
| ATOM | 839 | OD1 | ASP | | 121 | −1.325 | 84.095 | −37.333 | 1.00 | 22.47 | O |
| ATOM | 840 | OD2 | ASP | | 121 | −1.919 | 83.062 | −39.167 | 1.00 | 22.36 | O |
| ATOM | 841 | C | ASP | | 121 | −1.580 | 81.159 | −36.050 | 1.00 | 21.23 | C |
| ATOM | 842 | O | ASP | | 121 | −2.026 | 80.088 | −36.456 | 1.00 | 21.25 | O |
| ATOM | 843 | N | LEU | | 122 | −2.273 | 82.002 | −35.294 | 1.00 | 21.72 | N |
| ATOM | 844 | CA | LEU | | 122 | −3.638 | 81.738 | −34.860 | 1.00 | 22.17 | C |
| ATOM | 845 | CB | LEU | | 122 | −3.752 | 81.903 | −33.343 | 1.00 | 21.46 | C |
| ATOM | 846 | CG | LEU | | 122 | −3.003 | 80.887 | −32.480 | 1.00 | 21.02 | C |
| ATOM | 847 | CD1 | LEU | | 122 | −3.119 | 81.286 | −31.018 | 1.00 | 21.99 | C |
| ATOM | 848 | CD2 | LEU | | 122 | −3.573 | 79.496 | −32.712 | 1.00 | 21.00 | C |
| ATOM | 849 | C | LEU | | 122 | −4.623 | 82.683 | −35.525 | 1.00 | 24.02 | C |
| ATOM | 850 | O | LEU | | 122 | −5.833 | 82.555 | −35.342 | 1.00 | 24.81 | O |
| ATOM | 851 | N | GLY | | 123 | −4.109 | 83.633 | −36.298 | 1.00 | 25.02 | N |
| ATOM | 852 | CA | GLY | | 123 | −4.986 | 84.591 | −36.948 | 1.00 | 23.79 | C |
| ATOM | 853 | C | GLY | | 123 | −5.397 | 85.657 | −35.950 | 1.00 | 25.45 | C |
| ATOM | 854 | O | GLY | | 123 | −4.952 | 85.638 | −34.796 | 1.00 | 24.13 | O |
| ATOM | 855 | N | ALA | | 124 | −6.244 | 86.587 | −36.382 | 1.00 | 24.35 | N |
| ATOM | 856 | CA | ALA | | 124 | −6.698 | 87.663 | −35.510 | 1.00 | 24.82 | C |
| ATOM | 857 | CB | ALA | | 124 | −7.102 | 88.869 | −36.339 | 1.00 | 25.39 | C |
| ATOM | 858 | C | ALA | | 124 | −7.867 | 87.224 | −34.640 | 1.00 | 26.89 | C |
| ATOM | 859 | O | ALA | | 124 | −8.692 | 86.408 | −35.054 | 1.00 | 28.64 | O |
| ATOM | 860 | N | PHE | | 125 | −7.935 | 87.773 | −33.434 | 1.00 | 27.23 | N |
| ATOM | 861 | CA | PHE | | 125 | −9.007 | 87.451 | −32.506 | 1.00 | 28.93 | C |
| ATOM | 862 | CB | PHE | | 125 | −8.651 | 86.187 | −31.707 | 1.00 | 29.65 | C |
| ATOM | 863 | CG | PHE | | 125 | −7.347 | 86.280 | −30.943 | 1.00 | 30.27 | C |
| ATOM | 864 | CD1 | PHE | | 125 | −7.271 | 86.981 | −29.743 | 1.00 | 30.55 | C |
| ATOM | 865 | CD2 | PHE | | 125 | −6.204 | 85.640 | −31.415 | 1.00 | 29.95 | C |
| ATOM | 866 | CE1 | PHE | | 125 | −6.073 | 87.042 | −29.019 | 1.00 | 30.06 | C |
| ATOM | 867 | CE2 | PHE | | 125 | −5.002 | 85.693 | −30.702 | 1.00 | 31.22 | C |
| ATOM | 868 | CZ | PHE | | 125 | −4.937 | 86.395 | −29.500 | 1.00 | 28.65 | C |
| ATOM | 869 | C | PHE | | 125 | −9.286 | 88.623 | −31.571 | 1.00 | 29.58 | C |
| ATOM | 870 | O | PHE | | 125 | −8.449 | 89.514 | −31.406 | 1.00 | 30.38 | O |
| ATOM | 871 | N | SER | | 126 | −10.476 | 88.639 | −30.984 | 1.00 | 30.78 | N |
| ATOM | 872 | CA | SER | | 126 | −10.844 | 89.702 | −30.057 | 1.00 | 32.91 | C |
| ATOM | 873 | CB | SER | | 126 | −12.174 | 90.344 | −30.475 | 1.00 | 34.77 | C |
| ATOM | 874 | OG | SER | | 126 | −13.223 | 89.393 | −30.470 | 1.00 | 35.78 | O |
| ATOM | 875 | C | SER | | 126 | −10.965 | 89.082 | −28.670 | 1.00 | 32.39 | C |
| ATOM | 876 | O | SER | | 126 | −10.806 | 87.869 | −28.512 | 1.00 | 31.50 | O |
| ATOM | 877 | N | ARG | | 127 | −11.227 | 89.906 | −27.661 | 1.00 | 33.69 | N |
| ATOM | 878 | CA | ARG | | 127 | −11.362 | 89.380 | −26.311 | 1.00 | 34.29 | C |
| ATOM | 879 | CB | ARG | | 127 | −11.480 | 90.518 | −25.295 | 1.00 | 33.79 | C |
| ATOM | 880 | CG | ARG | | 127 | −10.154 | 91.169 | −24.925 | 1.00 | 33.56 | C |
| ATOM | 881 | CD | ARG | | 127 | −10.357 | 92.178 | −23.810 | 1.00 | 30.80 | C |
| ATOM | 882 | NE | ARG | | 127 | −9.126 | 92.850 | −23.398 | 1.00 | 31.57 | N |
| ATOM | 883 | CZ | ARG | | 127 | −8.138 | 92.282 | −22.707 | 1.00 | 28.99 | C |
| ATOM | 884 | NH1 | ARG | | 127 | −8.212 | 91.011 | −22.339 | 1.00 | 27.53 | N |
| ATOM | 885 | NH2 | ARG | | 127 | −7.078 | 93.003 | −22.363 | 1.00 | 29.56 | N |
| ATOM | 886 | C | ARG | | 127 | −12.586 | 88.479 | −26.224 | 1.00 | 35.91 | C |
| ATOM | 887 | O | ARG | | 127 | −13.568 | 88.674 | −26.942 | 1.00 | 36.34 | O |
| ATOM | 888 | N | GLY | | 128 | −12.511 | 87.477 | −25.358 | 1.00 | 36.68 | N |
| ATOM | 889 | CA | GLY | | 128 | −13.627 | 86.568 | −25.190 | 1.00 | 37.74 | C |
| ATOM | 890 | C | GLY | | 128 | −13.636 | 85.374 | −26.122 | 1.00 | 37.64 | C |
| ATOM | 891 | O | GLY | | 128 | −14.669 | 84.722 | −26.265 | 1.00 | 39.65 | O |
| ATOM | 892 | N | GLN | | 129 | −12.504 | 85.079 | −26.758 | 1.00 | 35.78 | N |
| ATOM | 893 | CA | GLN | | 129 | −12.426 | 83.940 | −27.670 | 1.00 | 34.33 | C |
| ATOM | 894 | CB | GLN | | 129 | −12.048 | 84.393 | −29.087 | 1.00 | 34.91 | C |
| ATOM | 895 | CG | GLN | | 129 | −12.884 | 85.531 | −29.661 | 1.00 | 36.32 | C |
| ATOM | 896 | CD | GLN | | 129 | −12.544 | 85.818 | −31.119 | 1.00 | 37.49 | C |
| ATOM | 897 | OE1 | GLN | | 129 | −12.893 | 86.866 | −31.656 | 1.00 | 39.45 | O |
| ATOM | 898 | NE2 | GLN | | 129 | −11.867 | 84.878 | −31.764 | 1.00 | 38.62 | N |
| ATOM | 899 | C | GLN | | 129 | −11.407 | 82.906 | −27.202 | 1.00 | 33.56 | C |
| ATOM | 900 | O | GLN | | 129 | −11.659 | 81.705 | −27.264 | 1.00 | 33.43 | O |
| ATOM | 901 | N | MET | | 130 | −10.253 | 83.378 | −26.735 | 1.00 | 32.43 | N |
| ATOM | 902 | CA | MET | | 130 | −9.186 | 82.493 | −26.274 | 1.00 | 30.95 | C |
| ATOM | 903 | CB | MET | | 130 | −7.829 | 83.068 | −26.696 | 1.00 | 32.56 | C |
| ATOM | 904 | CG | MET | | 130 | −7.726 | 83.415 | −28.176 | 1.00 | 32.35 | C |
| ATOM | 905 | SD | MET | | 130 | −7.822 | 81.957 | −29.211 | 1.00 | 36.35 | S |
| ATOM | 906 | CE | MET | | 130 | −7.294 | 82.602 | −30.794 | 1.00 | 35.60 | C |
| ATOM | 907 | C | MET | | 130 | −9.209 | 82.323 | −24.754 | 1.00 | 30.40 | C |
| ATOM | 908 | O | MET | | 130 | −9.865 | 83.086 | −24.048 | 1.00 | 30.69 | O |
| ATOM | 909 | N | GLN | | 131 | −8.496 | 81.319 | −24.252 | 1.00 | 29.34 | N |
| ATOM | 910 | CA | GLN | | 131 | −8.433 | 81.108 | −22.810 | 1.00 | 29.39 | C |
| ATOM | 911 | CB | GLN | | 131 | −7.512 | 79.931 | −22.493 | 1.00 | 31.38 | C |
| ATOM | 912 | CG | GLN | | 131 | −7.961 | 78.652 | −23.181 | 1.00 | 34.16 | C |
| ATOM | 913 | CD | GLN | | 131 | −7.066 | 77.471 | −22.887 | 1.00 | 35.47 | C |
| ATOM | 914 | OE1 | GLN | | 131 | −7.143 | 76.865 | −21.820 | 1.00 | 37.92 | O |
| ATOM | 915 | NE2 | GLN | | 131 | −6.202 | 77.137 | −23.837 | 1.00 | 38.00 | N |
| ATOM | 916 | C | GLN | | 131 | −7.911 | 82.416 | −22.208 | 1.00 | 29.40 | C |
| ATOM | 917 | O | GLN | | 131 | −7.004 | 83.050 | −22.755 | 1.00 | 27.15 | O |
| ATOM | 918 | N | LYS | | 132 | −8.497 | 82.818 | −21.087 | 1.00 | 27.37 | N |
| ATOM | 919 | CA | LYS | | 132 | −8.158 | 84.081 | −20.439 | 1.00 | 27.29 | C |
| ATOM | 920 | CB | LYS | | 132 | −8.864 | 84.159 | −19.078 | 1.00 | 25.74 | C |
| ATOM | 921 | CG | LYS | | 132 | −8.879 | 85.550 | −18.472 | 1.00 | 27.26 | C |
| ATOM | 922 | CD | LYS | | 132 | −9.508 | 86.578 | −19.406 | 1.00 | 25.54 | C |
| ATOM | 923 | CE | LYS | | 132 | −9.475 | 87.961 | −18.774 | 1.00 | 27.97 | C |
| ATOM | 924 | NZ | LYS | | 132 | −9.815 | 89.046 | −19.733 | 1.00 | 26.31 | N |
| ATOM | 925 | C | LYS | | 132 | −6.674 | 84.459 | −20.286 | 1.00 | 25.45 | C |
| ATOM | 926 | O | LYS | | 132 | −6.270 | 85.546 | −20.700 | 1.00 | 25.96 | O |
| ATOM | 927 | N | PRO | | 133 | −5.850 | 83.587 | −19.687 | 1.00 | 25.30 | N |
| ATOM | 928 | CD | PRO | | 133 | −6.108 | 82.234 | −19.164 | 1.00 | 25.57 | C |
| ATOM | 929 | CA | PRO | | 133 | −4.432 | 83.947 | −19.536 | 1.00 | 26.02 | C |
| ATOM | 930 | CB | PRO | | 133 | −3.808 | 82.674 | −18.962 | 1.00 | 25.81 | C |
| ATOM | 931 | CG | PRO | | 133 | −4.952 | 82.039 | −18.213 | 1.00 | 28.23 | C |
| ATOM | 932 | C | PRO | | 133 | −3.793 | 84.345 | −20.872 | 1.00 | 25.42 | C |
| ATOM | 933 | O | PRO | | 133 | −3.108 | 85.364 | −20.973 | 1.00 | 24.40 | O |
| ATOM | 934 | N | PHE | | 134 | −4.030 | 83.523 | −21.889 | 1.00 | 24.85 | N |
| ATOM | 935 | CA | PHE | | 134 | −3.498 | 83.754 | −23.230 | 1.00 | 22.30 | C |
| ATOM | 936 | CB | PHE | | 134 | −3.967 | 82.630 | −24.155 | 1.00 | 22.14 | C |
| ATOM | 937 | CG | PHE | | 134 | −3.269 | 82.598 | −25.487 | 1.00 | 18.96 | C |
| ATOM | 938 | CD1 | PHE | | 134 | −2.144 | 81.802 | −25.679 | 1.00 | 19.71 | C |
| ATOM | 939 | CD2 | PHE | | 134 | −3.738 | 83.360 | −26.551 | 1.00 | 19.52 | C |
| ATOM | 940 | CE1 | PHE | | 134 | −1.491 | 81.768 | −26.923 | 1.00 | 20.20 | C |
| ATOM | 941 | CE2 | PHE | | 134 | −3.092 | 83.332 | −27.791 | 1.00 | 18.91 | C |
| ATOM | 942 | CZ | PHE | | 134 | −1.969 | 82.535 | −27.973 | 1.00 | 18.42 | C |
| ATOM | 943 | C | PHE | | 134 | −4.005 | 85.099 | −23.748 | 1.00 | 22.35 | C |
| ATOM | 944 | O | PHE | | 134 | −3.235 | 85.923 | −24.256 | 1.00 | 21.63 | O |
| ATOM | 945 | N | GLU | | 135 | −5.312 | 85.317 | −23.612 | 1.00 | 20.90 | N |
| ATOM | 946 | CA | GLU | | 135 | −5.944 | 86.552 | −24.059 | 1.00 | 19.88 | C |
| ATOM | 947 | CB | GLU | | 135 | −7.442 | 86.543 | −23.721 | 1.00 | 19.66 | C |
| ATOM | 948 | CG | GLU | | 135 | −8.115 | 87.888 | −23.942 | 1.00 | 22.59 | C |
| ATOM | 949 | CD | GLU | | 135 | −9.577 | 87.887 | −23.527 | 1.00 | 24.18 | C |
| ATOM | 950 | OE1 | GLU | | 135 | −10.318 | 86.992 | −23.980 | 1.00 | 23.97 | O |
| ATOM | 951 | OE2 | GLU | | 135 | −9.975 | 88.783 | −22.755 | 1.00 | 25.76 | O |
| ATOM | 952 | C | GLU | | 135 | −5.317 | 87.796 | −23.447 | 1.00 | 19.84 | C |
| ATOM | 953 | O | GLU | | 135 | −4.905 | 88.716 | −24.161 | 1.00 | 18.70 | O |
| ATOM | 954 | N | ASP | | 136 | −5.267 | 87.840 | −22.120 | 1.00 | 18.82 | N |
| ATOM | 955 | CA | ASP | | 136 | −4.702 | 88.997 | −21.440 | 1.00 | 20.21 | C |
| ATOM | 956 | CB | ASP | | 136 | −4.791 | 88.828 | −19.919 | 1.00 | 22.82 | C |
| ATOM | 957 | CG | ASP | | 136 | −6.226 | 88.942 | −19.403 | 1.00 | 26.00 | C |
| ATOM | 958 | OD1 | ASP | | 136 | −7.021 | 89.701 | −20.008 | 1.00 | 25.89 | O |
| ATOM | 959 | OD2 | ASP | | 136 | −6.554 | 88.290 | −18.389 | 1.00 | 26.58 | O |
| ATOM | 960 | C | ASP | | 136 | −3.262 | 89.253 | −21.863 | 1.00 | 18.27 | C |
| ATOM | 961 | O | ASP | | 136 | −2.873 | 90.396 | −22.109 | 1.00 | 17.82 | O |
| ATOM | 962 | N | ALA | | 137 | −2.477 | 88.186 | −21.953 | 1.00 | 19.38 | N |
| ATOM | 963 | CA | ALA | | 137 | −1.085 | 88.313 | −22.370 | 1.00 | 19.02 | C |
| ATOM | 964 | CB | ALA | | 137 | −0.398 | 86.948 | −22.317 | 1.00 | 17.24 | C |
| ATOM | 965 | C | ALA | | 137 | −1.064 | 88.869 | −23.797 | 1.00 | 17.73 | C |
| ATOM | 966 | O | ALA | | 137 | −0.363 | 89.830 | −24.084 | 1.00 | 16.91 | O |
| ATOM | 967 | N | SER | | 138 | −1.862 | 88.278 | −24.682 | 1.00 | 18.80 | N |
| ATOM | 968 | CA | SER | | 138 | −1.918 | 88.726 | −26.075 | 1.00 | 19.09 | C |
| ATOM | 969 | CB | SER | | 138 | −2.959 | 87.924 | −26.863 | 1.00 | 17.22 | C |
| ATOM | 970 | OG | SER | | 138 | −2.599 | 86.560 | −26.929 | 1.00 | 16.63 | O |
| ATOM | 971 | C | SER | | 138 | −2.238 | 90.207 | −26.207 | 1.00 | 20.37 | C |
| ATOM | 972 | O | SER | | 138 | −1.594 | 90.917 | −26.977 | 1.00 | 18.45 | O |
| ATOM | 973 | N | PHE | | 139 | −3.232 | 90.677 | −25.457 | 1.00 | 21.44 | N |
| ATOM | 974 | CA | PHE | | 139 | −3.621 | 92.078 | −25.529 | 1.00 | 22.76 | C |
| ATOM | 975 | CB | PHE | | 139 | −5.071 | 92.261 | −25.058 | 1.00 | 23.84 | C |
| ATOM | 976 | CG | PHE | | 139 | −6.084 | 91.897 | −26.103 | 1.00 | 23.76 | C |
| ATOM | 977 | CD1 | PHE | | 139 | −6.465 | 90.573 | −26.293 | 1.00 | 24.31 | C |
| ATOM | 978 | CD2 | PHE | | 139 | −6.593 | 92.871 | −26.959 | 1.00 | 24.92 | C |
| ATOM | 979 | CE1 | PHE | | 139 | −7.338 | 90.220 | −27.330 | 1.00 | 25.59 | C |
| ATOM | 980 | CE2 | PHE | | 139 | −7.462 | 92.530 | −27.997 | 1.00 | 25.58 | C |
| ATOM | 981 | CZ | PHE | | 139 | −7.834 | 91.202 | −28.182 | 1.00 | 23.61 | C |
| ATOM | 982 | C | PHE | | 139 | −2.710 | 93.044 | −24.784 | 1.00 | 22.19 | C |
| ATOM | 983 | O | PHE | | 139 | −2.773 | 94.250 | −25.014 | 1.00 | 23.46 | O |
| ATOM | 984 | N | ALA | | 140 | −1.866 | 92.521 | −23.900 | 1.00 | 22.26 | N |
| ATOM | 985 | CA | ALA | | 140 | −0.944 | 93.365 | −23.145 | 1.00 | 22.10 | C |
| ATOM | 986 | CB | ALA | | 140 | −0.659 | 92.749 | −21.783 | 1.00 | 21.06 | C |
| ATOM | 987 | C | ALA | | 140 | 0.355 | 93.545 | −23.928 | 1.00 | 22.97 | C |
| ATOM | 988 | O | ALA | | 140 | 1.113 | 94.485 | −23.687 | 1.00 | 22.84 | O |
| ATOM | 989 | N | LEU | | 141 | 0.605 | 92.635 | −24.865 | 1.00 | 23.11 | N |
| ATOM | 990 | CA | LEU | | 141 | 1.797 | 92.703 | −25.702 | 1.00 | 24.24 | C |
| ATOM | 991 | CB | LEU | | 141 | 1.997 | 91.396 | −26.479 | 1.00 | 22.44 | C |
| ATOM | 992 | CG | LEU | | 141 | 2.413 | 90.128 | −25.740 | 1.00 | 22.58 | C |
| ATOM | 993 | CD1 | LEU | | 141 | 2.328 | 88.941 | −26.694 | 1.00 | 20.79 | C |
| ATOM | 994 | CD2 | LEU | | 141 | 3.830 | 90.282 | −25.201 | 1.00 | 22.42 | C |
| ATOM | 995 | C | LEU | | 141 | 1.643 | 93.828 | −26.710 | 1.00 | 26.34 | C |
| ATOM | 996 | O | LEU | | 141 | 0.531 | 94.138 | −27.147 | 1.00 | 27.56 | O |
| ATOM | 997 | N | ARG | | 142 | 2.760 | 94.444 | −27.072 | 1.00 | 27.25 | N |
| ATOM | 998 | CA | ARG | | 142 | 2.751 | 95.503 | −28.066 | 1.00 | 28.18 | C |
| ATOM | 999 | CB | ARG | | 142 | 3.857 | 96.521 | −27.757 | 1.00 | 32.17 | C |
| ATOM | 1000 | CG | ARG | | 142 | 3.759 | 97.143 | −26.350 | 1.00 | 35.93 | C |
| ATOM | 1001 | CD | ARG | | 142 | 4.898 | 98.126 | −26.081 | 1.00 | 41.00 | C |
| ATOM | 1002 | NE | ARG | | 142 | 6.204 | 97.468 | −26.056 | 1.00 | 44.51 | N |
| ATOM | 1003 | CZ | ARG | | 142 | 6.755 | 96.929 | −24.972 | 1.00 | 46.60 | C |
| ATOM | 1004 | NH1 | ARG | | 142 | 6.117 | 96.975 | −23.810 | 1.00 | 47.17 | N |
| ATOM | 1005 | NH2 | ARG | | 142 | 7.936 | 96.327 | −25.053 | 1.00 | 46.70 | N |
| ATOM | 1006 | C | ARG | | 142 | 3.026 | 94.773 | −29.382 | 1.00 | 27.39 | C |
| ATOM | 1007 | O | ARG | | 142 | 3.561 | 93.662 | −29.376 | 1.00 | 24.82 | O |
| ATOM | 1008 | N | THR | | 143 | 2.651 | 95.366 | −30.507 | 1.00 | 25.81 | N |
| ATOM | 1009 | CA | THR | | 143 | 2.893 | 94.713 | −31.785 | 1.00 | 25.66 | C |
| ATOM | 1010 | CB | THR | | 143 | 2.419 | 95.585 | −32.961 | 1.00 | 27.45 | C |
| ATOM | 1011 | OG1 | THR | | 143 | 0.989 | 95.629 | −32.962 | 1.00 | 31.20 | O |
| ATOM | 1012 | CG2 | THR | | 143 | 2.898 | 95.013 | −34.287 | 1.00 | 28.00 | C |
| ATOM | 1013 | C | THR | | 143 | 4.377 | 94.400 | −31.952 | 1.00 | 24.68 | C |
| ATOM | 1014 | O | THR | | 143 | 5.232 | 95.267 | −31.756 | 1.00 | 22.46 | O |
| ATOM | 1015 | N | GLY | | 144 | 4.672 | 93.146 | −32.288 | 1.00 | 21.98 | N |
| ATOM | 1016 | CA | GLY | | 144 | 6.047 | 92.725 | −32.483 | 1.00 | 22.85 | C |
| ATOM | 1017 | C | GLY | | 144 | 6.702 | 92.132 | −31.248 | 1.00 | 22.96 | C |
| ATOM | 1018 | O | GLY | | 144 | 7.758 | 91.501 | −31.342 | 1.00 | 23.14 | O |
| ATOM | 1019 | N | GLU | | 145 | 6.070 | 92.320 | −30.092 | 1.00 | 21.60 | N |
| ATOM | 1020 | CA | GLU | | 145 | 6.600 | 91.824 | −28.827 | 1.00 | 21.63 | C |
| ATOM | 1021 | CB | GLU | | 145 | 6.106 | 92.714 | −27.686 | 1.00 | 22.78 | C |
| ATOM | 1022 | CG | GLU | | 145 | 6.417 | 92.190 | −26.300 | 1.00 | 30.05 | C |
| ATOM | 1023 | CD | GLU | | 145 | 6.070 | 93.191 | −25.216 | 1.00 | 32.54 | C |
| ATOM | 1024 | OE1 | GLU | | 145 | 5.127 | 93.991 | −25.432 | 1.00 | 33.01 | O |
| ATOM | 1025 | OE2 | GLU | | 145 | 6.732 | 93.170 | −24.151 | 1.00 | 31.52 | O |
| ATOM | 1026 | C | GLU | | 145 | 6.270 | 90.362 | −28.532 | 1.00 | 21.30 | C |
| ATOM | 1027 | O | GLU | | 145 | 5.258 | 89.820 | −29.006 | 1.00 | 21.67 | O |
| ATOM | 1028 | N | MET | | 146 | 7.137 | 89.727 | −27.750 | 1.00 | 17.85 | N |
| ATOM | 1029 | CA | MET | | 146 | 6.964 | 88.329 | −27.383 | 1.00 | 18.52 | C |
| ATOM | 1030 | CB | MET | | 146 | 8.170 | 87.508 | −27.855 | 1.00 | 18.33 | C |
| ATOM | 1031 | CG | MET | | 146 | 8.036 | 86.006 | −27.647 | 1.00 | 18.45 | C |
| ATOM | 1032 | SD | MET | | 146 | 9.473 | 85.107 | −28.253 | 1.00 | 19.11 | S |
| ATOM | 1033 | CE | MET | | 146 | 10.611 | 85.343 | −26.885 | 1.00 | 17.93 | C |
| ATOM | 1034 | C | MET | | 146 | 6.796 | 88.195 | −25.871 | 1.00 | 17.61 | C |
| ATOM | 1035 | O | MET | | 146 | 7.380 | 88.953 | −25.091 | 1.00 | 15.74 | O |
| ATOM | 1036 | N | SER | | 147 | 5.993 | 87.222 | −25.463 | 1.00 | 15.54 | N |
| ATOM | 1037 | CA | SER | | 147 | 5.736 | 86.993 | −24.050 | 1.00 | 16.26 | C |
| ATOM | 1038 | CB | SER | | 147 | 4.361 | 86.352 | −23.868 | 1.00 | 15.11 | C |
| ATOM | 1039 | OG | SER | | 147 | 4.441 | 84.965 | −24.194 | 1.00 | 14.89 | O |
| ATOM | 1040 | C | SER | | 147 | 6.757 | 86.041 | −23.463 | 1.00 | 14.75 | C |
| ATOM | 1041 | O | SER | | 147 | 7.610 | 85.510 | −24.170 | 1.00 | 17.13 | O |
| ATOM | 1042 | N | GLY | | 148 | 6.662 | 85.846 | −22.152 | 1.00 | 15.66 | N |
| ATOM | 1043 | CA | GLY | | 148 | 7.510 | 84.885 | −21.475 | 1.00 | 16.34 | C |
| ATOM | 1044 | C | GLY | | 148 | 6.613 | 83.656 | −21.359 | 1.00 | 18.14 | C |
| ATOM | 1045 | O | GLY | | 148 | 5.650 | 83.536 | −22.119 | 1.00 | 18.35 | O |
| ATOM | 1046 | N | PRO | | 149 | 6.883 | 82.725 | −20.437 | 1.00 | 19.97 | N |
| ATOM | 1047 | CD | PRO | | 149 | 8.083 | 82.538 | −19.604 | 1.00 | 19.15 | C |
| ATOM | 1048 | CA | PRO | | 149 | 5.994 | 81.560 | −20.354 | 1.00 | 19.60 | C |
| ATOM | 1049 | CB | PRO | | 149 | 6.705 | 80.648 | −19.357 | 1.00 | 19.40 | C |
| ATOM | 1050 | CG | PRO | | 149 | 8.155 | 81.036 | −19.499 | 1.00 | 19.39 | C |
| ATOM | 1051 | C | PRO | | 149 | 4.606 | 81.976 | −19.858 | 1.00 | 20.03 | C |
| ATOM | 1052 | O | PRO | | 149 | 4.490 | 82.648 | −18.835 | 1.00 | 19.61 | O |
| ATOM | 1053 | N | VAL | | 150 | 3.564 | 81.591 | −20.592 | 1.00 | 18.37 | N |
| ATOM | 1054 | CA | VAL | | 150 | 2.185 | 81.911 | −20.204 | 1.00 | 17.89 | C |
| ATOM | 1055 | CB | VAL | | 150 | 1.476 | 82.752 | −21.289 | 1.00 | 16.88 | C |
| ATOM | 1056 | CG1 | VAL | | 150 | −0.003 | 82.912 | −20.953 | 1.00 | 17.72 | C |
| ATOM | 1057 | CG2 | VAL | | 150 | 2.139 | 84.123 | −21.392 | 1.00 | 18.15 | C |
| ATOM | 1058 | C | VAL | | 150 | 1.441 | 80.585 | −19.994 | 1.00 | 18.54 | C |
| ATOM | 1059 | O | VAL | | 150 | 1.385 | 79.745 | −20.893 | 1.00 | 20.63 | O |
| ATOM | 1060 | N | PHE | | 151 | 0.880 | 80.403 | −18.802 | 1.00 | 18.16 | N |
| ATOM | 1061 | CA | PHE | | 151 | 0.187 | 79.166 | −18.466 | 1.00 | 19.83 | C |
| ATOM | 1062 | CB | PHE | | 151 | 0.457 | 78.802 | −17.001 | 1.00 | 18.89 | C |
| ATOM | 1063 | CG | PHE | | 151 | 1.919 | 78.736 | −16.645 | 1.00 | 19.97 | C |
| ATOM | 1064 | CD1 | PHE | | 151 | 2.670 | 79.901 | −16.516 | 1.00 | 21.49 | C |
| ATOM | 1065 | CD2 | PHE | | 151 | 2.543 | 77.510 | −16.448 | 1.00 | 20.07 | C |
| ATOM | 1066 | CE1 | PHE | | 151 | 4.030 | 79.846 | −16.193 | 1.00 | 21.89 | C |
| ATOM | 1067 | CE2 | PHE | | 151 | 3.907 | 77.441 | −16.123 | 1.00 | 20.62 | C |
| ATOM | 1068 | CZ | PEE | | 151 | 4.647 | 78.610 | −15.996 | 1.00 | 21.08 | C |
| ATOM | 1069 | C | PHE | | 151 | −1.324 | 79.198 | −18.689 | 1.00 | 20.80 | C |
| ATOM | 1070 | O | PHE | | 151 | −1.990 | 80.183 | −18.379 | 1.00 | 22.68 | O |
| ATOM | 1071 | N | THR | | 152 | −1.849 | 78.111 | −19.243 | 1.00 | 22.86 | N |
| ATOM | 1072 | CA | THR | | 152 | −3.285 | 77.957 | −19.480 | 1.00 | 22.63 | C |
| ATOM | 1073 | CB | THR | | 152 | −3.693 | 78.253 | −20.941 | 1.00 | 22.02 | C |
| ATOM | 1074 | OG1 | THR | | 152 | −3.093 | 77.272 | −21.798 | 1.00 | 20.71 | O |
| ATOM | 1075 | CG2 | THR | | 152 | −3.271 | 79.643 | −21.358 | 1.00 | 20.29 | C |
| ATOM | 1076 | C | THR | | 152 | −3.543 | 76.477 | −19.251 | 1.00 | 21.73 | C |
| ATOM | 1077 | O | THR | | 152 | −2.602 | 75.705 | −19.048 | 1.00 | 21.87 | O |
| ATOM | 1078 | N | ASP | | 153 | −4.805 | 76.068 | −19.307 | 1.00 | 24.33 | N |
| ATOM | 1079 | CA | ASP | | 153 | −5.123 | 74.658 | −19.126 | 1.00 | 25.99 | C |
| ATOM | 1080 | CB | ASP | | 153 | −6.631 | 74.477 | −18.965 | 1.00 | 30.01 | C |
| ATOM | 1081 | CG | ASP | | 153 | −7.112 | 74.928 | −17.610 | 1.00 | 35.09 | C |
| ATOM | 1082 | OD1 | ASP | | 153 | −6.560 | 74.422 | −16.612 | 1.00 | 37.74 | O |
| ATOM | 1083 | OD2 | ASP | | 153 | −8.023 | 75.781 | −17.535 | 1.00 | 39.10 | O |
| ATOM | 1084 | C | ASP | | 153 | −4.606 | 73.796 | −20.271 | 1.00 | 25.66 | C |
| ATOM | 1085 | O | ASP | | 153 | −4.556 | 72.572 | −20.162 | 1.00 | 25.55 | O |
| ATOM | 1086 | N | SER | | 154 | −4.223 | 74.434 | −21.375 | 1.00 | 24.11 | N |
| ATOM | 1087 | CA | SER | | 154 | −3.696 | 73.701 | −22.519 | 1.00 | 22.95 | C |
| ATOM | 1088 | CB | SER | | 154 | −3.823 | 74.527 | −23.803 | 1.00 | 23.27 | C |
| ATOM | 1089 | OG | SER | | 154 | −5.179 | 74.756 | −24.119 | 1.00 | 27.06 | O |
| ATOM | 1090 | C | SER | | 154 | −2.229 | 73.353 | −22.302 | 1.00 | 21.74 | C |
| ATOM | 1091 | O | SER | | 154 | −1.741 | 72.348 | −22.810 | 1.00 | 21.26 | O |
| ATOM | 1092 | N | GLY | | 155 | −1.534 | 74.190 | −21.541 | 1.00 | 21.35 | N |
| ATOM | 1093 | CA | GLY | | 155 | −0.124 | 73.957 | −21.285 | 1.00 | 20.66 | C |
| ATOM | 1094 | C | GLY | | 155 | 0.600 | 75.278 | −21.088 | 1.00 | 20.14 | C |
| ATOM | 1095 | O | GLY | | 155 | 0.043 | 76.211 | −20.505 | 1.00 | 21.33 | O |
| ATOM | 1096 | N | ILE | | 156 | 1.838 | 75.360 | −21.566 | 1.00 | 19.48 | N |
| ATOM | 1097 | CA | ILE | | 156 | 2.624 | 76.585 | −21.434 | 1.00 | 17.83 | C |
| ATOM | 1098 | CB | ILE | | 156 | 3.964 | 76.314 | −20.731 | 1.00 | 19.45 | C |
| ATOM | 1099 | CG2 | ILE | | 156 | 4.685 | 77.638 | −20.452 | 1.00 | 20.57 | C |
| ATOM | 1100 | CG1 | ILE | | 156 | 3.717 | 75.565 | −19.418 | 1.00 | 21.12 | C |
| ATOM | 1101 | CD1 | ILE | | 156 | 4.996 | 75.084 | −18.742 | 1.00 | 21.13 | C |
| ATOM | 1102 | C | ILE | | 156 | 2.903 | 77.153 | −22.823 | 1.00 | 17.34 | C |
| ATOM | 1103 | O | ILE | | 156 | 3.376 | 76.442 | −23.712 | 1.00 | 15.78 | O |
| ATOM | 1104 | N | HIS | | 157 | 2.623 | 78.442 | −22.995 | 1.00 | 17.88 | N |
| ATOM | 1105 | CA | HIS | | 157 | 2.812 | 79.104 | −24.285 | 1.00 | 17.22 | C |
| ATOM | 1106 | CB | HIS | | 157 | 1.532 | 79.813 | −24.735 | 1.00 | 17.03 | C |
| ATOM | 1107 | CG | HIS | | 157 | 0.270 | 79.032 | −24.545 | 1.00 | 21.13 | C |
| ATOM | 1108 | CD2 | HIS | | 157 | −0.401 | 78.673 | −23.422 | 1.00 | 21.81 | C |
| ATOM | 1109 | ND1 | HIS | | 157 | −0.518 | 78.635 | −25.605 | 1.00 | 19.22 | N |
| ATOM | 1110 | CE1 | HIS | | 157 | −1.622 | 78.073 | −25.145 | 1.00 | 22.18 | C |
| ATOM | 1111 | NE2 | HIS | | 157 | −1.577 | 78.084 | −23.824 | 1.00 | 21.37 | N |
| ATOM | 1112 | C | HIS | | 157 | 3.878 | 80.199 | −24.317 | 1.00 | 17.58 | C |
| ATOM | 1113 | O | HIS | | 157 | 4.198 | 80.816 | −23.297 | 1.00 | 16.44 | O |
| ATOM | 1114 | N | ILE | | 158 | 4.389 | 80.431 | −25.522 | 1.00 | 16.72 | N |
| ATOM | 1115 | CA | ILE | | 158 | 5.302 | 81.521 | −25.815 | 1.00 | 17.11 | C |
| ATOM | 1116 | CB | ILE | | 158 | 6.594 | 81.076 | −26.512 | 1.00 | 18.65 | C |
| ATOM | 1117 | CG2 | ILE | | 158 | 7.356 | 82.314 | −26.995 | 1.00 | 18.68 | C |
| ATOM | 1118 | CG1 | ILE | | 158 | 7.470 | 80.275 | −25.549 | 1.00 | 19.23 | C |
| ATOM | 1119 | CD1 | ILE | | 158 | 8.655 | 79.600 | −26.228 | 1.00 | 19.68 | C |
| ATOM | 1120 | C | ILE | | 158 | 4.412 | 82.218 | −26.841 | 1.00 | 17.26 | C |
| ATOM | 1121 | O | ILE | | 158 | 3.994 | 81.594 | −27.823 | 1.00 | 18.55 | O |
| ATOM | 1122 | N | ILE | | 159 | 4.092 | 83.486 | −26.610 | 1.00 | 16.20 | N |
| ATOM | 1123 | CA | ILE | | 159 | 3.206 | 84.213 | −27.515 | 1.00 | 16.72 | C |
| ATOM | 1124 | CB | ILE | | 159 | 1.991 | 84.782 | −26.750 | 1.00 | 15.63 | C |
| ATOM | 1125 | CG2 | ILE | | 159 | 1.007 | 85.438 | −27.725 | 1.00 | 15.56 | C |
| ATOM | 1126 | CG1 | ILE | | 159 | 1.324 | 83.659 | −25.950 | 1.00 | 18.35 | C |
| ATOM | 1127 | CD1 | ILE | | 159 | 0.233 | 84.131 | −25.010 | 1.00 | 13.68 | C |
| ATOM | 1128 | C | ILE | | 159 | 3.882 | 85.369 | −28.236 | 1.00 | 17.33 | C |
| ATOM | 1129 | O | ILE | | 159 | 4.592 | 86.161 | −27.623 | 1.00 | 17.96 | O |
| ATOM | 1130 | N | LEU | | 160 | 3.640 | 85.462 | −29.540 | 1.00 | 16.08 | N |
| ATOM | 1131 | CA | LEU | | 160 | 4.204 | 86.542 | −30.348 | 1.00 | 17.81 | C |
| ATOM | 1132 | CB | LEU | | 160 | 5.076 | 85.974 | −31.473 | 1.00 | 15.82 | C |
| ATOM | 1133 | CG | LEU | | 160 | 5.670 | 87.024 | −32.419 | 1.00 | 16.76 | C |
| ATOM | 1134 | CD1 | LEU | | 160 | 6.699 | 87.847 | −31.668 | 1.00 | 15.36 | C |
| ATOM | 1135 | CD2 | LEU | | 160 | 6.322 | 86.354 | −33.619 | 1.00 | 17.05 | C |
| ATOM | 1136 | C | LEU | | 160 | 3.071 | 87.345 | −30.967 | 1.00 | 16.24 | C |
| ATOM | 1137 | O | LEU | | 160 | 2.278 | 86.805 | −31.728 | 1.00 | 16.17 | O |
| ATOM | 1138 | N | ARG | | 161 | 2.969 | 88.626 | −30.637 | 1.00 | 18.92 | N |
| ATOM | 1139 | CA | ARG | | 161 | 1.911 | 89.426 | −31.234 | 1.00 | 20.23 | C |
| ATOM | 1140 | CB | ARG | | 161 | 1.482 | 90.575 | −30.322 | 1.00 | 20.66 | C |
| ATOM | 1141 | CG | ARG | | 161 | 0.3.73 | 91.405 | −30.955 | 1.00 | 21.45 | C |
| ATOM | 1142 | CD | ARG | | 161 | −0.061 | 92.588 | −30.106 | 1.00 | 22.90 | C |
| ATOM | 1143 | NE | ARG | | 161 | −0.886 | 93.485 | −30.913 | 1.00 | 24.24 | N |
| ATOM | 1144 | CZ | ARG | | 161 | −1.254 | 94.706 | −30.543 | 1.00 | 27.93 | C |
| ATOM | 1145 | NH1 | ARG | | 161 | −0.873 | 95.185 | −29.363 | 1.00 | 27.83 | N |
| ATOM | 1146 | NH2 | ARG | | 161 | −1.983 | 95.457 | −31.364 | 1.00 | 25.63 | N |
| ATOM | 1147 | C | ARG | | 161 | 2.445 | 89.969 | −32.551 | 1.00 | 19.80 | C |
| ATOM | 1148 | O | ARG | | 161 | 3.473 | 90.645 | −32.584 | 1.00 | 20.36 | O |
| ATOM | 1149 | N | THR | | 162 | 1.744 | 89.663 | −33.634 | 1.00 | 20.07 | N |
| ATOM | 1150 | CA | THR | | 162 | 2.165 | 90.086 | −34.961 | 1.00 | 21.67 | C |
| ATOM | 1151 | CB | THR | | 162 | 2.086 | 88.907 | −35.931 | 1.00 | 21.24 | C |
| ATOM | 1152 | OG1 | THR | | 162 | 0.756 | 88.373 | −35.919 | 1.00 | 21.27 | O |
| ATOM | 1153 | CG2 | THR | | 162 | 3.065 | 87.815 | −35.512 | 1.00 | 17.97 | C |
| ATOM | 1154 | C | THR | | 162 | 1.354 | 91.249 | −35.529 | 1.00 | 23.61 | C |
| ATOM | 1155 | O | THR | | 162 | 1.780 | 91.890 | −36.488 | 1.00 | 23.31 | O |
| ATOM | 1156 | N | GLU | | 163 | 0.191 | 91.514 | −34.938 | 1.00 | 24.13 | N |
| ATOM | 1157 | CA | GLU | | 163 | −0.679 | 92.598 | −35.389 | 1.00 | 25.11 | C |
| ATOM | 1158 | CB | GLU | | 163 | −1.600 | 92.106 | −36.514 | 1.00 | 26.50 | C |
| ATOM | 1159 | CG | GLU | | 163 | −0.922 | 91.890 | −37.856 | 1.00 | 27.37 | C |
| ATOM | 1160 | CD | GLU | | 163 | −1.885 | 91.359 | −38.911 | 1.00 | 30.99 | C |
| ATOM | 1161 | OE1 | GLU | | 163 | −3.095 | 91.655 | −38.818 | 1.00 | 31.76 | O |
| ATOM | 1162 | OE2 | GLU | | 163 | −1.431 | 90.655 | −39.839 | 1.00 | 32.55 | O |
| ATOM | 1163 | C | GLU | | 163 | −1.538 | 93.128 | −34.248 | 1.00 | 24.59 | C |
| ATOM | 1164 | O | GLU | | 163 | −1.771 | 92.371 | −33.285 | 1.00 | 23.98 | O |
| ATOM | 1165 | OXT | GLU | | 163 | −1.995 | 94.285 | −34.344 | 1.00 | 23.92 | O |
| ATOM | 1166 | OH2 | TIP | S | 1 | 4.996 | 92.293 | −22.205 | 1.00 | 22.33 | O |
| ATOM | 1167 | OH2 | TIP | S | 2 | 3.904 | 94.668 | −23.331 | 1.00 | 19.09 | O |
| ATOM | 1168 | OH2 | TIP | S | 3 | 16.835 | 89.300 | −27.847 | 1.00 | 18.64 | O |
| ATOM | 1169 | OH2 | TIP | S | 4 | 4.478 | 71.586 | −38.273 | 1.00 | 17.60 | O |
| ATOM | 1170 | OH2 | TIP | S | 5 | 1.493 | 76.589 | −32.561 | 1.00 | 18.71 | O |
| ATOM | 1171 | OH2 | TIP | S | 6 | 2.829 | 86.799 | −13.992 | 1.00 | 22.50 | O |
| ATOM | 1172 | OH2 | TIP | S | 7 | 14.735 | 85.536 | −32.999 | 1.00 | 17.41 | O |
| ATOM | 1173 | OH2 | TIP | S | 8 | 1.586 | 86.170 | −38.741 | 1.00 | 26.18 | O |
| ATOM | 1174 | OH2 | TIP | S | 9 | 14.294 | 73.884 | −25.020 | 1.00 | 23.10 | O |
| ATOM | 1175 | OH2 | TIP | S | 10 | 4.098 | 82.829 | −36.928 | 1.00 | 21.12 | O |
| ATOM | 1176 | OH2 | TIP | S | 11 | −9.802 | 85.994 | −26.475 | 1.00 | 29.89 | O |
| ATOM | 1177 | OH2 | TIP | S | 12 | 5.050 | 76.024 | −40.565 | 1.00 | 22.93 | O |
| ATOM | 1178 | OH2 | TIP | S | 13 | 14.963 | 90.911 | −23.596 | 1.00 | 25.00 | O |
| ATOM | 1179 | OH2 | TIP | S | 14 | 5.367 | 66.693 | −27.487 | 1.00 | 24.18 | O |
| ATOM | 1180 | OH2 | TIP | S | 15 | 6.032 | 63.967 | −23.108 | 1.00 | 29.08 | O |
| ATOM | 1181 | OH2 | TIP | S | 16 | 2.596 | 76.938 | −43.445 | 1.00 | 20.23 | O |
| ATOM | 1182 | OH2 | TIP | S | 17 | 18.067 | 98.333 | −17.353 | 1.00 | 24.92 | O |
| ATOM | 1183 | OH2 | TIP | S | 18 | 9.702 | 89.713 | −30.296 | 1.00 | 26.29 | O |
| ATOM | 1184 | OH2 | TIP | S | 19 | −12.052 | 88.072 | −20.923 | 1.00 | 29.06 | O |
| ATOM | 1185 | OH2 | TIP | S | 20 | 12.519 | 69.724 | −20.067 | 1.00 | 29.52 | O |
| ATOM | 1186 | OH2 | TIP | S | 21 | 5.362 | 77.311 | −42.993 | 1.00 | 27.16 | O |
| ATOM | 1187 | OH2 | TIP | S | 22 | 16.494 | 93.043 | −23.048 | 1.00 | 26.53 | O |
| ATOM | 1188 | OH2 | TIP | S | 23 | 14.077 | 81.918 | −21.032 | 1.00 | 23.49 | O |
| ATOM | 1189 | OH2 | TIP | S | 24 | 6.543 | 82.880 | −16.782 | 1.00 | 26.43 | O |
| ATOM | 1190 | OH2 | TIP | S | 25 | 10.451 | 86.570 | −17.616 | 1.00 | 27.70 | O |
| ATOM | 1191 | OH2 | TIP | S | 26 | 3.027 | 59.344 | −30.431 | 1.00 | 33.40 | O |
| ATOM | 1192 | OH2 | TIP | S | 27 | 13.001 | 70.485 | −28.343 | 1.00 | 29.47 | O |
| ATOM | 1193 | OH2 | TIP | S | 28 | −4.495 | 92.277 | −21.189 | 1.00 | 31.94 | O |
| ATOM | 1194 | OH2 | TIP | S | 29 | −4.163 | 71.408 | −32.025 | 1.00 | 27.78 | O |
| ATOM | 1195 | OH2 | TIP | S | 30 | −2.003 | 81.843 | −41.579 | 1.00 | 35.12 | O |
| ATOM | 1196 | OH2 | TIP | S | 31 | −11.542 | 92.565 | −28.064 | 1.00 | 33.33 | O |
| ATOM | 1197 | OH2 | TIP | S | 32 | −2.782 | 69.641 | −29.548 | 1.00 | 30.94 | O |
| ATOM | 1198 | OH2 | TIP | S | 33 | 0.077 | 74.916 | −17.705 | 1.00 | 27.54 | O |
| ATOM | 1199 | OH2 | TIP | S | 34 | −1.798 | 86.275 | −18.776 | 1.00 | 22.76 | O |
| ATOM | 1200 | OH2 | TIP | S | 35 | 11.527 | 82.271 | −19.663 | 1.00 | 29.95 | O |
| ATOM | 1201 | OH2 | TIP | S | 36 | 12.435 | 69.156 | −35.685 | 1.00 | 36.02 | O |
| ATOM | 1202 | OH2 | TIP | S | 37 | 6.525 | 70.164 | −27.535 | 1.00 | 26.81 | O |
| ATOM | 1203 | OH2 | TIP | S | 38 | 15.072 | 88.089 | −18.216 | 1.00 | 25.26 | O |
| ATOM | 1204 | OH2 | TIP | S | 39 | 8.927 | 70.472 | −26.831 | 1.00 | 26.75 | O |
| ATOM | 1205 | OH2 | TIP | S | 40 | −4.554 | 56.096 | −32.538 | 1.00 | 46.41 | O |
| ATOM | 1206 | OH2 | TIP | S | 41 | −6.592 | 78.691 | −19.130 | 1.00 | 34.05 | O |
| ATOM | 1207 | OH2 | TIP | S | 42 | 10.941 | 90.094 | −11.523 | 1.00 | 31.06 | O |
| ATOM | 1208 | OH2 | TIP | S | 43 | 27.416 | 94.285 | −11.592 | 1.00 | 39.24 | O |
| ATOM | 1209 | OH2 | TIP | S | 44 | 17.021 | 88.279 | −11.350 | 1.00 | 39.39 | O |
| ATOM | 1210 | OH2 | TIP | S | 45 | −11.320 | 84.727 | −22.664 | 1.00 | 37.94 | O |
| ATOM | 1211 | OH2 | TIP | S | 46 | 8.130 | 90.624 | −34.026 | 1.00 | 29.86 | O |
| ATOM | 1212 | OH2 | TIP | S | 47 | −1.729 | 68.721 | −32.242 | 1.00 | 29.50 | O |
| ATOM | 1213 | OH2 | TIP | S | 48 | −4.411 | 94.820 | −34.154 | 1.00 | 31.74 | O |
| ATOM | 1214 | OH2 | TIP | S | 49 | 16.939 | 96.485 | −24.679 | 1.00 | 37.27 | O |
| ATOM | 1215 | OH2 | TIP | S | 50 | 0.991 | 96.551 | −21.899 | 1.00 | 33.01 | O |
| ATOM | 1216 | OH2 | TIP | S | 51 | 2.693 | 58.746 | −27.460 | 1.00 | 40.54 | O |
| ATOM | 1217 | OH2 | TIP | S | 52 | 11.179 | 73.774 | −14.605 | 1.00 | 35.96 | O |
| ATOM | 1218 | OH2 | TIP | S | 53 | 20.243 | 79.681 | −26.059 | 1.00 | 44.12 | O |
| ATOM | 1219 | OH2 | TIP | S | 54 | −1.551 | 88.145 | −14.461 | 1.00 | 41.34 | O |
| ATOM | 1220 | OH2 | TIP | S | 55 | 10.812 | 79.660 | −40.306 | 1.00 | 41.96 | O |
| ATOM | 1221 | OH2 | TIP | S | 56 | 9.581 | 91.050 | −27.357 | 1.00 | 36.77 | O |
| ATOM | 1222 | OH2 | TIP | S | 57 | 0.588 | 85.609 | −36.254 | 1.00 | 20.89 | O |
| ATOM | 1223 | OH2 | TIP | S | 58 | 1.467 | 56.895 | −31.356 | 1.00 | 36.77 | O |
| ATOM | 1224 | OH2 | TIP | S | 59 | −7.051 | 71.283 | −35.374 | 1.00 | 44.19 | O |
| ATOM | 1225 | OH2 | TIP | S | 60 | 10.725 | 87.273 | −30.581 | 1.00 | 20.71 | O |
| ATOM | 1226 | OH2 | TIP | S | 61 | 23.232 | 91.784 | −19.476 | 1.00 | 26.11 | O |
| ATOM | 1227 | OH2 | TIP | S | 62 | −4.934 | 58.687 | −29.339 | 1.00 | 35.95 | O |
| ATOM | 1228 | OH2 | TIP | S | 63 | 9.871 | 87.603 | −12.492 | 1.00 | 30.66 | O |
| ATOM | 1229 | OH2 | TIP | S | 64 | −13.005 | 84.066 | −20.570 | 1.00 | 31.49 | O |
| ATOM | 1230 | OH2 | TIP | S | 65 | 8.069 | 66.785 | −33.871 | 1.00 | 37.90 | O |
| ATOM | 1231 | OH2 | TIP | S | 66 | 7.786 | 80.984 | −15.179 | 1.00 | 32.21 | O |
| ATOM | 1232 | OH2 | TIP | S | 67 | −3.967 | 87.413 | −16.543 | 1.00 | 35.89 | O |
| ATOM | 1233 | OH2 | TIP | S | 68 | 15.843 | 75.405 | −20.027 | 1.00 | 32.99 | O |
| ATOM | 1234 | OH2 | TIP | S | 69 | 18.704 | 92.052 | −24.094 | 1.00 | 40.34 | O |
| ATOM | 1235 | OH2 | TIP | S | 70 | 14.924 | 71.626 | −26.622 | 1.00 | 39.48 | O |
| ATOM | 1236 | OH2 | TIP | S | 71 | −7.578 | 73.953 | −21.960 | 1.00 | 36.90 | O |
| ATOM | 1237 | OH2 | TIP | S | 72 | 4.357 | 63.739 | −35.563 | 1.00 | 36.35 | O |
| ATOM | 1238 | OH2 | TIP | S | 73 | −0.680 | 96.464 | −26.110 | 1.00 | 40.17 | O |
| ATOM | 1239 | OH2 | TIP | S | 74 | 14.838 | 89.361 | −10.347 | 1.00 | 47.16 | O |
| ATOM | 1240 | OH2 | TIP | S | 75 | 4.185 | 93.296 | −8.477 | 1.00 | 46.61 | O |
| ATOM | 1241 | OH2 | TIP | S | 76 | 9.639 | 67.060 | −13.355 | 1.00 | 42.21 | O |
| ATOM | 1242 | OH2 | TIP | S | 77 | −8.619 | 91.119 | −17.955 | 1.00 | 45.22 | O |
| ATOM | 1243 | OH2 | TIP | S | 78 | 11.431 | 67.104 | −21.172 | 1.00 | 43.05 | O |
| ATOM | 1244 | OH2 | TIP | S | 79 | −10.529 | 81.014 | −19.998 | 1.00 | 40.81 | O |
| ATOM | 1245 | OH2 | TIP | S | 80 | 18.262 | 74.953 | −25.341 | 1.00 | 45.73 | O |
| ATOM | 1246 | OH2 | TIP | S | 81 | −8.270 | 83.821 | −35.626 | 1.00 | 42.43 | O |
| ATOM | 1247 | OH2 | TIP | S | 82 | −5.257 | 92.334 | −36.516 | 1.00 | 44.54 | O |
| ATOM | 1248 | OH2 | TIP | S | 83 | 24.875 | 97.135 | −9.923 | 1.00 | 48.63 | O |
| ATOM | 1249 | OH2 | TIP | S | 84 | −10.775 | 82.437 | −32.093 | 1.00 | 53.25 | O |
| ATOM | 1250 | OH2 | TIP | S | 85 | 2.471 | 67.905 | −14.830 | 1.00 | 37.86 | O |
| ATOM | 1251 | OH2 | TIP | S | 86 | 15.900 | 102.036 | −16.997 | 1.00 | 45.47 | O |
| ATOM | 1252 | OH2 | TIP | S | 87 | 18.249 | 83.052 | −19.151 | 1.00 | 40.32 | O |
| ATOM | 1253 | OH2 | TIP | S | 88 | 21.041 | 79.609 | −19.141 | 1.00 | 45.55 | O |
| ATOM | 1254 | OH2 | TIP | S | 89 | 2.124 | 58.830 | −33.333 | 1.00 | 44.53 | O |
| ATOM | 1255 | OH2 | TIP | S | 90 | 10.828 | 83.746 | −17.756 | 1.00 | 44.80 | O |
| ATOM | 1256 | OH2 | TIP | S | 91 | 15.547 | 73.007 | −22.889 | 1.00 | 43.13 | O |
| ATOM | 1257 | OH2 | TIP | S | 92 | 15.824 | 81.889 | −19.101 | 1.00 | 33.52 | O |
| ATOM | 1258 | OH2 | TIP | S | 93 | −4.678 | 89.942 | −39.858 | 1.00 | 47.08 | O |
| ATOM | 1259 | OH2 | TIP | S | 94 | 12.133 | 89.060 | −27.026 | 1.00 | 40.68 | O |
| ATOM | 1260 | OH2 | TIP | S | 95 | 13.939 | 80.272 | −39.801 | 1.00 | 42.43 | O |
| ATOM | 1261 | OH2 | TIP | S | 96 | 13.282 | 91.087 | −25.735 | 1.00 | 54.38 | O |
| ATOM | 1262 | OH2 | TIP | S | 97 | 9.728 | 93.063 | −8.513 | 1.00 | 46.78 | O |
| ATOM | 1263 | OH2 | TIP | S | 98 | 10.268 | 67.985 | −26.895 | 1.00 | 49.19 | O |
| ATOM | 1264 | OH2 | TIP | S | 99 | 5.654 | 84.634 | −38.615 | 1.00 | 47.56 | O |
| ATOM | 1265 | OH2 | TIP | S | 100 | −8.209 | 77.763 | −27.166 | 1.00 | 41.45 | O |
| ATOM | 1266 | OH2 | TIP | S | 101 | 15.842 | 72.276 | −34.464 | 1.00 | 45.45 | O |
| ATOM | 1267 | OH2 | TIP | S | 102 | 0.992 | 98.097 | −30.418 | 1.00 | 47.28 | O |
| ATOM | 1268 | OH2 | TIP | S | 103 | 14.928 | 70.921 | −21.453 | 1.00 | 46.03 | O |
| ATOM | 1269 | OH2 | TIP | S | 104 | 21.961 | 80.057 | −36.566 | 1.00 | 54.72 | O |
| ATOM | 1270 | OH2 | TIP | S | 105 | −9.589 | 77.974 | −19.455 | 1.00 | 39.81 | O |
| ATOM | 1271 | OH2 | TIP | S | 106 | 20.556 | 88.719 | −14.791 | 1.00 | 37.91 | O |
| ATOM | 1272 | OH2 | TIP | S | 107 | −3.922 | 71.217 | −13.348 | 1.00 | 36.43 | O |
| ATOM | 1273 | OH2 | TIP | S | 108 | 2.761 | 99.624 | −15.558 | 1.00 | 49.03 | O |
| ATOM | 1274 | OH2 | TIP | S | 109 | 20.532 | 99.746 | −13.057 | 1.00 | 40.66 | O |
| ATOM | 1275 | OH2 | TIP | S | 110 | 2.200 | 88.512 | −39.451 | 1.00 | 42.35 | O |
| ATOM | 1276 | OH2 | TIP | S | 111 | −0.243 | 98.079 | −33.871 | 1.00 | 45.52 | O |
| ATOM | 1277 | OH2 | TIP | S | 112 | 2.345 | 94.561 | −37.605 | 1.00 | 47.43 | O |
| ATOM | 1278 | OH2 | TIP | S | 113 | 9.704 | 74.745 | −12.514 | 1.00 | 48.37 | O |
| ATOM | 1279 | O1 | PGA | B | 180 | 16.285 | 89.067 | −15.776 | 1.00 | 34.19 | O |
| ATOM | 1280 | C2 | PGA | B | 180 | 17.040 | 90.193 | −15.926 | 1.00 | 30.20 | C |
| ATOM | 1281 | C3 | PGA | B | 180 | 16.661 | 90.929 | −17.214 | 1.00 | 29.59 | C |
| ATOM | 1282 | O4 | PGA | B | 180 | 17.026 | 90.220 | −18.272 | 1.00 | 29.69 | O |
| ATOM | 1283 | C5 | PGA | B | 180 | 16.716 | 90.812 | −19.431 | 1.00 | 26.84 | C |
| ATOM | 1284 | C6 | PGA | B | 180 | 17.182 | 89.912 | −20.595 | 1.00 | 28.92 | C |
| ATOM | 1285 | O7 | PGA | B | 180 | 16.724 | 88.650 | −20.399 | 1.00 | 27.81 | O |
| ATOM | 1286 | C8 | PGA | B | 180 | 17.086 | 87.798 | −21.370 | 1.00 | 25.04 | C |
| ATOM | 1287 | C9 | PGA | B | 180 | 16.507 | 86.410 | −21.040 | 1.00 | 25.59 | C |
| ATOM | 1288 | O10 | PGA | B | 180 | 15.136 | 86.455 | −21.050 | 1.00 | 25.09 | O |
| ATOM | 1289 | C11 | PGA | B | 180 | 14.598 | 85.259 | −20.760 | 1.00 | 24.29 | C |
| ATOM | 1290 | C12 | PGA | B | 180 | 13.117 | 85.435 | −20.368 | 1.00 | 25.67 | C |
| ATOM | 1291 | O13 | PGA | B | 180 | 12.444 | 86.021 | −21.365 | 1.00 | 23.85 | O |
| ATOM | 1292 | C14 | PGA | B | 180 | 11.161 | 86.166 | −21.029 | 1.00 | 29.38 | C |
| ATOM | 1293 | C15 | PGA | B | 180 | 10.391 | 86.849 | −22.179 | 1.00 | 30.98 | C |
| ATOM | 1294 | O16 | PGA | B | 180 | 10.795 | 88.116 | −22.353 | 1.00 | 31.74 | O |
| ATOM | 1295 | C17 | PGA | B | 180 | 10.277 | 88.508 | −23.469 | 1.00 | 37.46 | C |
| ATOM | 1296 | C18 | PGA | B | 180 | 10.644 | 89.938 | −23.815 | 1.00 | 40.16 | C |
| ATOM | 1297 | O19 | PGA | B | 180 | 10.068 | 90.217 | −24.949 | 1.00 | 44.29 | O |
| ATOM | 1298 | O1 | PGB | C | 181 | −6.751 | 79.793 | −25.879 | 1.00 | 38.35 | O |
| ATOM | 1299 | C2 | PGB | C | 181 | −5.559 | 80.213 | −26.288 | 1.00 | 40.88 | C |
| ATOM | 1300 | C3 | PGB | C | 181 | −5.052 | 79.372 | −27.484 | 1.00 | 40.71 | C |
| ATOM | 1301 | O4 | PGB | C | 181 | −5.160 | 78.075 | −27.211 | 1.00 | 44.58 | O |
| ATOM | 1302 | C5 | PGB | C | 181 | −4.724 | 77.341 | −28.259 | 1.00 | 45.07 | C |
| ATOM | 1303 | C6 | PGB | C | 181 | −4.847 | 75.836 | −27.942 | 1.00 | 47.07 | C |
| ATOM | 1304 | O7 | PGB | C | 181 | −3.638 | 75.359 | −27.580 | 1.00 | 46.85 | O |
| ATOM | 1305 | O8 | C21 | | 21 | −10.942 | 69.468 | −28.934 | 1.00 | 64.01 | O |
| ATOM | 1306 | C7 | C21 | | 21 | −10.997 | 69.859 | −27.769 | 1.00 | 64.20 | C |
| ATOM | 1307 | C9 | C21 | | 21 | −9.695 | 70.185 | −27.002 | 1.00 | 62.13 | C |
| ATOM | 1308 | C10 | C21 | | 21 | −9.789 | 70.641 | −25.634 | 1.00 | 62.02 | C |
| ATOM | 1309 | C11 | C21 | | 21 | −11.161 | 70.801 | −24.958 | 1.00 | 63.36 | C |
| ATOM | 1310 | C4 | C21 | | 21 | −12.451 | 70.474 | −25.729 | 1.00 | 64.92 | C |
| ATOM | 1311 | C5 | C21 | | 21 | −13.735 | 70.618 | −25.107 | 1.00 | 65.55 | C |
| ATOM | 1312 | C6 | C21 | | 21 | −14.926 | 70.313 | −25.829 | 1.00 | 66.30 | C |
| ATOM | 1313 | C1 | C21 | | 21 | −14.847 | 69.861 | −27.179 | 1.00 | 66.65 | C |
| ATOM | 1314 | C2 | C21 | | 21 | −13.577 | 69.714 | −27.808 | 1.00 | 66.21 | C |
| ATOM | 1315 | C3 | C21 | | 21 | −12.370 | 70.016 | −27.094 | 1.00 | 65.16 | C |
| ATOM | 1316 | O12 | C21 | | 21 | −11.228 | 71.190 | −23.796 | 1.00 | 63.77 | O |
| ATOM | 1317 | C13 | C21 | | 21 | −8.598 | 70.947 | −24.906 | 1.00 | 59.33 | C |
| ATOM | 1318 | C14 | C21 | | 21 | −7.313 | 70.807 | −25.508 | 1.00 | 56.59 | C |
| ATOM | 1319 | S10 | C21 | | 21 | −5.832 | 71.191 | −24.585 | 1.00 | 52.91 | S |
| ATOM | 1320 | O20 | C21 | | 21 | −5.051 | 69.986 | −24.421 | 1.00 | 51.47 | O |
| ATOM | 1321 | O21 | C21 | | 21 | −6.252 | 71.876 | −23.366 | 1.00 | 52.93 | O |
| ATOM | 1322 | O22 | C21 | | 21 | −5.143 | 72.153 | −25.516 | 1.00 | 52.55 | O |
| ATOM | 1323 | C15 | C21 | | 21 | −7.212 | 70.356 | −26.860 | 1.00 | 58.26 | C |
| ATOM | 1324 | O18 | C21 | | 21 | −5.949 | 70.220 | −27.450 | 1.00 | 55.87 | O |
| ATOM | 1325 | C16 | C21 | | 21 | −8.391 | 70.045 | −27.607 | 1.00 | 60.54 | C |
| ATOM | 1326 | O17 | C21 | | 21 | −8.226 | 69.607 | −28.931 | 1.00 | 60.45 | O |

Pin1 R14A/Pin-420 Interactions

The crystal structure of the co-complex with PIN-420 (the coordinate set is set forth in Table 3), shows interaction with Met130, Gln131, Ser154, and Cys113. PIN-420 alters the local protein environment by disrupting the hydrogen bond between Gln131 and Ser154. The normal hydrogen bond contact between Gln131 and Ser154 is disrupted, and each residue now interacts with PIN-420.

TABLE 3
Set of atomic coordinates of Pin1 R14A with PIN-420 bound in the active site.

| | | | | | | | | | | | | |
|---|---|---|---|---|---|---|---|---|---|---|---|---|
| ATOM | 1 | CB | LEU | | 7 | 56.132 | 105.496 | −69.819 | 1.00 | 45.12 | A | C |
| ATOM | 2 | CG | LEU | | 7 | 57.034 | 104.380 | −69.260 | 1.00 | 46.13 | A | C |
| ATOM | 3 | CD1 | LEU | | 7 | 56.338 | 103.659 | −68.119 | 1.00 | 45.79 | A | C |
| ATOM | 4 | CD2 | LEU | | 7 | 57.393 | 103.419 | −70.380 | 1.00 | 46.32 | A | C |
| ATOM | 5 | C | LEU | | 7 | 55.509 | 107.385 | −71.286 | 1.00 | 43.21 | A | C |
| ATOM | 6 | O | LEU | | 7 | 54.972 | 108.259 | −70.628 | 1.00 | 42.24 | A | O |
| ATOM | 7 | N | LEU | | 7 | 57.559 | 107.542 | −69.911 | 1.00 | 44.95 | A | N |
| ATOM | 8 | CA | LEU | | 7 | 56.688 | 106.614 | −70.713 | 1.00 | 44.20 | A | C |
| ATOM | 9 | N | PRO | | 8 | 55.070 | 107.039 | −72.507 | 1.00 | 42.79 | A | N |
| ATOM | 10 | CD | PRO | | 8 | 55.541 | 105.904 | −73.315 | 1.00 | 43.31 | A | C |
| ATOM | 11 | CA | PRO | | 8 | 53.936 | 107.710 | −73.157 | 1.00 | 42.15 | A | C |
| ATOM | 12 | CB | PRO | | 8 | 53.768 | 106.942 | −74.473 | 1.00 | 42.75 | A | C |
| ATOM | 13 | CG | PRO | | 8 | 55.102 | 106.306 | −74.706 | 1.00 | 43.37 | A | C |
| ATOM | 14 | C | PRO | | 8 | 52.652 | 107.664 | −72.298 | 1.00 | 41.60 | A | C |
| ATOM | 15 | O | PRO | | 8 | 52.526 | 106.865 | −71.349 | 1.00 | 42.16 | A | O |
| ATOM | 16 | N | PRO | | 9 | 51.672 | 108.517 | −72.635 | 1.00 | 40.32 | A | N |
| ATOM | 17 | CD | PRO | | 9 | 51.701 | 109.388 | −73.824 | 1.00 | 39.53 | A | C |
| ATOM | 18 | CA | PRO | | 9 | 50.383 | 108.631 | −71.933 | 1.00 | 37.44 | A | C |
| ATOM | 19 | CB | PRO | | 9 | 49.554 | 109.522 | −72.865 | 1.00 | 37.63 | A | C |
| ATOM | 20 | CG | PRO | | 9 | 50.589 | 110.383 | −73.506 | 1.00 | 39.51 | A | C |
| ATOM | 21 | C | PRO | | 9 | 49.672 | 107.315 | −71.632 | 1.00 | 35.64 | A | C |
| ATOM | 22 | O | PRO | | 9 | 49.372 | 106.522 | −72.540 | 1.00 | 33.85 | A | O |
| ATOM | 23 | N | GLY | | 10 | 49.405 | 107.101 | −70.347 | 1.00 | 33.56 | A | N |
| ATOM | 24 | CA | GLY | | 10 | 48.704 | 105.915 | −69.913 | 1.00 | 30.60 | A | C |
| ATOM | 25 | C | GLY | | 10 | 49.552 | 104.722 | −69.495 | 1.00 | 27.82 | A | C |
| ATOM | 26 | O | GLY | | 10 | 49.045 | 103.863 | −68.802 | 1.00 | 24.06 | A | O |
| ATOM | 27 | N | TRP | | 11 | 50.823 | 104.692 | −69.897 | 1.00 | 28.45 | A | N |
| ATOM | 28 | CA | TRP | | 11 | 51.703 | 103.550 | −69.584 | 1.00 | 29.12 | A | C |
| ATOM | 29 | CB | TRP | | 11 | 52.855 | 103.473 | −70.593 | 1.00 | 27.79 | A | C |
| ATOM | 30 | CG | TRP | | 11 | 52.402 | 103.131 | −71.965 | 1.00 | 26.17 | A | C |
| ATOM | 31 | CD2 | TRP | | 11 | 52.070 | 101.830 | −72.458 | 1.00 | 26.93 | A | C |
| ATOM | 32 | CE2 | TRP | | 11 | 51.698 | 101.980 | −73.821 | 1.00 | 26.12 | A | C |
| ATOM | 33 | CE3 | TRP | | 11 | 52.050 | 100.547 | −71.883 | 1.00 | 26.02 | A | C |
| ATOM | 34 | CD1 | TRP | | 11 | 52.213 | 103.993 | −73.011 | 1.00 | 26.11 | A | C |
| ATOM | 35 | NE1 | TRP | | 11 | 51.791 | 103.311 | −74.125 | 1.00 | 25.99 | A | N |
| ATOM | 36 | CZ2 | TRP | | 11 | 51.321 | 100.912 | −74.614 | 1.00 | 25.96 | A | C |
| ATOM | 37 | CZ3 | TRP | | 11 | 51.668 | 99.481 | −72.668 | 1.00 | 25.07 | A | C |
| ATOM | 38 | CH2 | TRP | | 11 | 51.309 | 99.664 | −74.029 | 1.00 | 28.08 | A | C |
| ATOM | 39 | C | TRP | | 11 | 52.262 | 103.474 | −68.170 | 1.00 | 29.92 | A | C |
| ATOM | 40 | O | TRP | | 11 | 52.443 | 104.490 | −67.499 | 1.00 | 29.95 | A | O |
| ATOM | 41 | N | GLU | | 12 | 52.481 | 102.240 | −67.696 | 1.00 | 28.77 | A | N |
| ATOM | 42 | CA | GLU | | 12 | 53.039 | 101.986 | −66.364 | 1.00 | 29.22 | A | C |
| ATOM | 43 | CB | GLU | | 12 | 51.949 | 101.951 | −65.274 | 1.00 | 31.03 | A | C |
| ATOM | 44 | CG | GLU | | 12 | 51.000 | 100.767 | −65.327 | 1.00 | 38.21 | A | C |
| ATOM | 45 | CD | GLU | | 12 | 50.013 | 100.740 | −64.153 | 1.00 | 42.00 | A | C |
| ATOM | 46 | OE1 | GLU | | 12 | 49.103 | 101.604 | −64.086 | 1.00 | 43.94 | A | O |
| ATOM | 47 | OE2 | GLU | | 12 | 50.157 | 99.851 | −63.289 | 1.00 | 45.85 | A | O |
| ATOM | 48 | C | GLU | | 12 | 53.777 | 100.625 | −66.382 | 1.00 | 29.39 | A | C |
| ATOM | 49 | O | GLU | | 12 | 53.521 | 99.765 | −67.235 | 1.00 | 25.12 | A | O |
| ATOM | 50 | N | LYS | | 13 | 54.682 | 100.450 | −65.435 | 1.00 | 29.42 | A | N |
| ATOM | 51 | CA | LYS | | 13 | 55.449 | 99.218 | −65.360 | 1.00 | 31.07 | A | C |
| ATOM | 52 | CB | LYS | | 13 | 56.809 | 99.484 | −64.735 | 1.00 | 35.41 | A | C |
| ATOM | 53 | CG | LYS | | 13 | 57.914 | 99.623 | −65.756 | 1.00 | 39.53 | A | C |
| ATOM | 54 | CD | LYS | | 13 | 59.281 | 99.665 | −65.050 | 1.00 | 44.11 | A | C |
| ATOM | 55 | CE | LYS | | 13 | 60.412 | 99.472 | −66.042 | 1.00 | 46.04 | A | C |
| ATOM | 56 | NZ | LYS | | 13 | 61.717 | 99.242 | −65.346 | 1.00 | 49.58 | A | N |
| ATOM | 57 | C | LYS | | 13 | 54.715 | 98.234 | −64.521 | 1.00 | 30.31 | A | C |
| ATOM | 58 | O | LYS | | 13 | 54.050 | 98.611 | −63.562 | 1.00 | 31.03 | A | O |
| ATOM | 59 | N | ALA | | 14 | 54.809 | 96.966 | −64.887 | 1.00 | 29.23 | A | N |
| ATOM | 60 | CA | ALA | | 14 | 54.158 | 95.921 | −64.124 | 1.00 | 28.03 | A | C |
| ATOM | 61 | CB | ALA | | 14 | 52.819 | 95.520 | −64.783 | 1.00 | 29.63 | A | C |
| ATOM | 62 | C | ALA | | 14 | 55.119 | 94.726 | −64.066 | 1.00 | 28.45 | A | C |
| ATOM | 63 | O | ALA | | 14 | 56.160 | 94.703 | −64.724 | 1.00 | 24.39 | A | O |
| ATOM | 64 | N | MET | | 15 | 54.738 | 93.730 | −63.290 | 1.00 | 30.12 | A | N |
| ATOM | 65 | CA | MET | | 15 | 55.560 | 92.542 | −63.137 | 1.00 | 31.95 | A | C |
| ATOM | 66 | CB | MET | | 15 | 55.989 | 92.429 | −61.686 | 1.00 | 36.28 | A | C |
| ATOM | 67 | CG | MET | | 15 | 56.718 | 91.147 | −61.387 | 1.00 | 42.59 | A | C |
| ATOM | 68 | SD | MET | | 15 | 58.409 | 91.363 | −61.896 | 1.00 | 49.52 | A | S |
| ATOM | 69 | CE | MET | | 15 | 58.932 | 92.794 | −60.774 | 1.00 | 47.65 | A | C |
| ATOM | 70 | C | MET | | 15 | 54.729 | 91.327 | −63.486 | 1.00 | 30.33 | A | C |
| ATOM | 71 | O | MET | | 15 | 53.636 | 91.172 | −62.943 | 1.00 | 27.69 | A | O |
| ATOM | 72 | N | SER | | 16 | 55.234 | 90.472 | −64.373 | 1.00 | 29.39 | A | N |
| ATOM | 73 | CA | SER | | 16 | 54.512 | 89.245 | −64.732 | 1.00 | 30.10 | A | C |
| ATOM | 74 | CB | SER | | 16 | 55.280 | 88.449 | −65.787 | 1.00 | 29.88 | A | C |
| ATOM | 75 | OG | SER | | 16 | 54.726 | 87.135 | −65.920 | 1.00 | 30.65 | A | O |
| ATOM | 76 | C | SER | | 16 | 54.340 | 88.347 | −63.504 | 1.00 | 31.27 | A | C |
| ATOM | 77 | O | SER | | 16 | 55.302 | 88.104 | −62.777 | 1.00 | 30.51 | A | O |
| ATOM | 78 | N | ARG | | 17 | 53.133 | 87.845 | −63.278 | 1.00 | 33.49 | A | N |
| ATOM | 79 | CA | ARG | | 17 | 52.886 | 86.962 | −62.137 | 1.00 | 36.89 | A | C |
| ATOM | 80 | CB | ARG | | 17 | 51.389 | 86.837 | −61.869 | 1.00 | 38.43 | A | C |
| ATOM | 81 | CG | ARG | | 17 | 50.741 | 88.067 | −61.307 | 1.00 | 40.79 | A | C |
| ATOM | 82 | CD | ARG | | 17 | 51.150 | 88.344 | −59.876 | 1.00 | 42.01 | A | C |
| ATOM | 83 | NE | ARG | | 17 | 50.731 | 89.695 | −59.494 | 1.00 | 44.30 | A | N |
| ATOM | 84 | CZ | ARG | | 17 | 49.501 | 90.039 | −59.099 | 1.00 | 44.50 | A | C |
| ATOM | 85 | NH1 | ARG | | 17 | 48.518 | 89.133 | −58.997 | 1.00 | 43.97 | A | N |
| ATOM | 86 | NH2 | ARG | | 17 | 49.245 | 91.316 | −58.862 | 1.00 | 44.67 | A | N |
| ATOM | 87 | C | ARG | | 17 | 53.436 | 85.563 | −62.390 | 1.00 | 38.51 | A | C |
| ATOM | 88 | O | ARG | | 17 | 53.601 | 84.767 | −61.467 | 1.00 | 38.21 | A | O |
| ATOM | 89 | N | SER | | 18 | 53.691 | 85.259 | −63.657 | 1.00 | 39.74 | A | N |
| ATOM | 90 | CA | SER | | 18 | 54.200 | 83.945 | −64.054 | 1.00 | 41.01 | A | C |
| ATOM | 91 | CB | SER | | 18 | 53.646 | 83.560 | −65.416 | 1.00 | 41.75 | A | C |
| ATOM | 92 | OG | SER | | 18 | 52.278 | 83.311 | −65.310 | 1.00 | 43.95 | A | O |
| ATOM | 93 | C | SER | | 18 | 55.699 | 83.849 | −64.151 | 1.00 | 41.06 | A | C |
| ATOM | 94 | O | SER | | 18 | 56.310 | 82.911 | −63.626 | 1.00 | 42.04 | A | O |
| ATOM | 95 | N | SER | | 19 | 56.285 | 84.823 | −64.832 | 1.00 | 39.54 | A | N |
| ATOM | 96 | CA | SER | | 19 | 57.711 | 84.835 | −65.068 | 1.00 | 38.42 | A | C |
| ATOM | 97 | CB | SER | | 19 | 57.967 | 85.227 | −66.518 | 1.00 | 39.80 | A | C |
| ATOM | 98 | OG | SER | | 19 | 57.269 | 86.410 | −66.852 | 1.00 | 39.69 | A | O |
| ATOM | 99 | C | SER | | 19 | 58.503 | 85.741 | −64.181 | 1.00 | 37.48 | A | C |
| ATOM | 100 | O | SER | | 19 | 59.690 | 85.512 | −63.965 | 1.00 | 38.01 | A | O |
| ATOM | 101 | N | GLY | | 20 | 57.861 | 86.784 | −63.667 | 1.00 | 36.08 | A | N |
| ATOM | 102 | CA | GLY | | 20 | 58.588 | 87.722 | −62.840 | 1.00 | 33.43 | A | C |
| ATOM | 103 | C | GLY | | 20 | 59.238 | 88.762 | −63.752 | 1.00 | 32.49 | A | C |
| ATOM | 104 | O | GLY | | 20 | 59.903 | 89.695 | −63.291 | 1.00 | 32.53 | A | O |
| ATOM | 105 | N | ARG | | 21 | 59.067 | 89.611 | −65.061 | 1.00 | 29.79 | A | N |
| ATOM | 106 | CA | ARG | | 21 | 59.657 | 89.569 | −65.972 | 1.00 | 27.70 | A | C |
| ATOM | 107 | CB | ARG | | 21 | 59.785 | 88.950 | −67.360 | 1.00 | 31.60 | A | C |
| ATOM | 108 | CG | ARG | | 21 | 61.140 | 89.185 | −68.040 | 1.00 | 34.43 | A | C |
| ATOM | 109 | CD | ARG | | 21 | 61.105 | 88.693 | −69.511 | 1.00 | 34.59 | A | C |
| ATOM | 110 | NE | ARG | | 21 | 60.242 | 87.525 | −69.635 | 1.00 | 35.91 | A | N |
| ATOM | 111 | CZ | ARG | | 21 | 59.346 | 87.342 | −70.603 | 1.00 | 35.58 | A | C |
| ATOM | 112 | NH1 | ARG | | 21 | 59.181 | 88.248 | −71.575 | 1.00 | 33.21 | A | N |
| ATOM | 113 | NH2 | ARG | | 21 | 58.563 | 86.267 | −70.553 | 1.00 | 35.31 | A | N |
| ATOM | 114 | C | ARG | | 21 | 58.804 | 90.860 | −66.037 | 1.00 | 25.50 | A | C |
| ATOM | 115 | O | ARG | | 21 | 57.578 | 90.830 | −65.926 | 1.00 | 24.14 | A | O |
| ATOM | 116 | N | VAL | | 22 | 59.444 | 91.997 | −66.203 | 1.00 | 23.36 | A | N |
| ATOM | 117 | CA | VAL | | 22 | 58.645 | 93.220 | −66.250 | 1.00 | 23.66 | A | C |
| ATOM | 118 | CB | VAL | | 22 | 59.516 | 94.432 | −66.039 | 1.00 | 24.20 | A | C |
| ATOM | 119 | CG1 | VAL | | 22 | 60.220 | 94.327 | −64.678 | 1.00 | 28.27 | A | C |
| ATOM | 120 | CG2 | VAL | | 22 | 60.556 | 94.517 | −67.131 | 1.00 | 27.40 | A | C |
| ATOM | 121 | C | VAL | | 22 | 57.909 | 93.361 | −67.593 | 1.00 | 21.54 | A | C |
| ATOM | 122 | O | VAL | | 22 | 58.359 | 92.847 | −68.615 | 1.00 | 20.98 | A | O |
| ATOM | 123 | N | TYR | | 23 | 56.752 | 94.011 | −67.580 | 1.00 | 20.65 | A | N |
| ATOM | 124 | CA | TYR | | 23 | 56.055 | 94.252 | −68.833 | 1.00 | 19.71 | A | C |
| ATOM | 125 | CB | TYR | | 23 | 55.008 | 93.177 | −69.134 | 1.00 | 18.59 | A | C |
| ATOM | 126 | CG | TYR | | 23 | 53.838 | 93.150 | −68.215 | 1.00 | 21.16 | A | C |
| ATOM | 127 | CD1 | TYR | | 23 | 52.634 | 93.751 | −68.583 | 1.00 | 20.99 | A | C |
| ATOM | 128 | CE1 | TYR | | 23 | 51.518 | 93.667 | −67.767 | 1.00 | 24.50 | A | C |
| ATOM | 129 | CD2 | TYR | | 23 | 53.907 | 92.473 | −66.995 | 1.00 | 22.06 | A | C |
| ATOM | 130 | CE2 | TYR | | 23 | 52.797 | 92.384 | −66.168 | 1.00 | 26.15 | A | C |
| ATOM | 131 | CZ | TYR | | 23 | 51.600 | 92.987 | −66.574 | 1.00 | 25.60 | A | C |
| ATOM | 132 | OH | TYR | | 23 | 50.482 | 92.902 | −65.796 | 1.00 | 26.07 | A | O |
| ATOM | 133 | C | TYR | | 23 | 55.431 | 95.654 | −68.682 | 1.00 | 21.65 | A | C |
| ATOM | 134 | O | TYR | | 23 | 55.613 | 96.317 | −67.654 | 1.00 | 20.50 | A | O |
| ATOM | 135 | N | TYR | | 24 | 54.723 | 96.103 | −69.699 | 1.00 | 19.39 | A | N |
| ATOM | 136 | CA | TYR | | 24 | 54.159 | 97.448 | −69.629 | 1.00 | 21.72 | A | C |
| ATOM | 137 | CB | TYR | | 24 | 54.825 | 98.315 | −70.692 | 1.00 | 24.00 | A | C |
| ATOM | 138 | CG | TYR | | 24 | 56.310 | 98.454 | −70.413 | 1.00 | 28.86 | A | C |
| ATOM | 139 | CD1 | TYR | | 24 | 57.224 | 97.476 | −70.832 | 1.00 | 31.55 | A | C |
| ATOM | 140 | CE1 | TYR | | 24 | 58.574 | 97.543 | −70.471 | 1.00 | 33.94 | A | C |
| ATOM | 141 | CD2 | TYR | | 24 | 56.776 | 99.501 | −69.637 | 1.00 | 31.11 | A | C |
| ATOM | 142 | CE2 | TYR | | 24 | 58.120 | 99.593 | −69.268 | 1.00 | 34.88 | A | C |
| ATOM | 143 | CZ | TYR | | 24 | 59.009 | 98.608 | −69.688 | 1.00 | 36.23 | A | C |
| ATOM | 144 | OH | TYR | | 24 | 60.327 | 98.714 | −69.307 | 1.00 | 38.97 | A | O |
| ATOM | 145 | C | TYR | | 24 | 52.661 | 97.336 | −69.844 | 1.00 | 19.03 | A | C |
| ATOM | 146 | O | TYR | | 24 | 52.220 | 96.531 | −70.655 | 1.00 | 18.17 | A | O |
| ATOM | 147 | N | PHE | | 25 | 51.903 | 98.136 | −69.104 | 1.00 | 19.96 | A | N |
| ATOM | 148 | CA | PHE | | 25 | 50.444 | 98.104 | −69.165 | 1.00 | 19.89 | A | C |
| ATOM | 149 | CB | PHE | | 25 | 49.924 | 97.559 | −67.837 | 1.00 | 21.97 | A | C |
| ATOM | 150 | CG | PHE | | 25 | 48.456 | 97.671 | −67.686 | 1.00 | 22.65 | A | C |
| ATOM | 151 | CD1 | PHE | | 25 | 47.609 | 96.890 | −68.479 | 1.00 | 25.04 | A | C |
| ATOM | 152 | CD2 | PHE | | 25 | 47.901 | 98.611 | −66.804 | 1.00 | 24.92 | A | C |
| ATOM | 153 | CE1 | PHE | | 25 | 46.183 | 97.026 | −68.429 | 1.00 | 25.30 | A | C |
| ATOM | 154 | CE2 | PHE | | 25 | 46.490 | 98.764 | −66.736 | 1.00 | 25.29 | A | C |
| ATOM | 155 | CZ | PHE | | 25 | 45.634 | 97.965 | −67.560 | 1.00 | 26.73 | A | C |
| ATOM | 156 | C | PHE | | 25 | 49.997 | 99.559 | −69.322 | 1.00 | 20.33 | A | C |
| ATOM | 157 | O | PHE | | 25 | 50.577 | 100.460 | −68.705 | 1.00 | 21.29 | A | O |
| ATOM | 158 | N | ASN | | 26 | 48.978 | 99.782 | −70.135 | 1.00 | 19.99 | A | N |
| ATOM | 159 | CA | ASN | | 26 | 48.442 | 101.156 | −70.343 | 1.00 | 20.03 | A | C |
| ATOM | 160 | CB | ASN | | 26 | 48.400 | 101.486 | −71.838 | 1.00 | 20.35 | A | C |
| ATOM | 161 | CG | ASN | | 26 | 48.007 | 102.939 | −72.093 | 1.00 | 20.30 | A | C |
| ATOM | 162 | OD1 | ASN | | 26 | 46.923 | 103.373 | −71.680 | 1.00 | 18.74 | A | O |
| ATOM | 163 | ND2 | ASN | | 26 | 48.877 | 103.675 | −72.774 | 1.00 | 20.10 | A | N |
| ATOM | 164 | C | ASN | | 26 | 47.057 | 101.185 | −69.738 | 1.00 | 17.03 | A | C |
| ATOM | 165 | O | ASN | | 26 | 46.151 | 100.450 | −70.158 | 1.00 | 16.51 | A | O |
| ATOM | 166 | N | HIS | | 27 | 46.871 | 102.024 | −68.724 | 1.00 | 16.46 | A | N |
| ATOM | 167 | CA | HIS | | 27 | 45.566 | 102.040 | −68.038 | 1.00 | 17.94 | A | C |
| ATOM | 168 | CB | HIS | | 27 | 45.674 | 102.626 | −66.619 | 1.00 | 17.75 | A | C |
| ATOM | 169 | CG | HIS | | 27 | 46.254 | 104.000 | −66.577 | 1.00 | 21.48 | A | C |
| ATOM | 170 | CD2 | HIS | | 27 | 45.692 | 105.216 | −66.790 | 1.00 | 23.71 | A | C |
| ATOM | 171 | ND1 | HIS | | 27 | 47.571 | 104.240 | −66.242 | 1.00 | 23.59 | A | N |
| ATOM | 172 | CE1 | HIS | | 27 | 47.798 | 105.543 | −66.257 | 1.00 | 25.25 | A | C |
| ATOM | 173 | NE2 | HIS | | 27 | 46.675 | 106.157 | −66.584 | 1.00 | 25.44 | A | N |
| ATOM | 174 | C | HIS | | 27 | 44.460 | 102.798 | −68.771 | 1.00 | 17.80 | A | C |
| ATOM | 175 | O | HIS | | 27 | 43.352 | 102.865 | −68.254 | 1.00 | 18.70 | A | O |
| ATOM | 176 | N | ILE | | 28 | 44.772 | 103.365 | −69.931 | 1.00 | 17.75 | A | N |
| ATOM | 177 | CA | ILE | | 28 | 43.760 | 104.044 | −70.724 | 1.00 | 18.73 | A | C |
| ATOM | 178 | CB | ILE | | 28 | 44.277 | 105.334 | −71.292 | 1.00 | 18.90 | A | C |
| ATOM | 179 | CG2 | ILE | | 28 | 43.156 | 106.025 | −72.071 | 1.00 | 18.20 | A | C |
| ATOM | 180 | CG1 | ILE | | 28 | 44.799 | 106.222 | −70.173 | 1.00 | 19.60 | A | C |
| ATOM | 181 | CD1 | ILE | | 28 | 45.624 | 107.418 | −70.705 | 1.00 | 19.60 | A | C |
| ATOM | 182 | C | ILE | | 28 | 43.274 | 103.159 | −71.896 | 1.00 | 18.50 | A | C |
| ATOM | 183 | O | ILE | | 28 | 42.087 | 103.139 | −72.213 | 1.00 | 19.56 | A | O |
| ATOM | 184 | N | THR | | 29 | 44.169 | 102.391 | −72.507 | 1.00 | 17.97 | A | N |
| ATOM | 185 | CA | THR | | 29 | 43.768 | 101.520 | −73.629 | 1.00 | 17.64 | A | C |
| ATOM | 186 | CB | THR | | 29 | 44.836 | 101.548 | −74.708 | 1.00 | 17.16 | A | C |
| ATOM | 187 | OG1 | THR | | 29 | 46.006 | 100.939 | −74.155 | 1.00 | 16.12 | A | O |
| ATOM | 188 | CG2 | THR | | 29 | 45.180 | 102.982 | −75.147 | 1.00 | 18.18 | A | C |
| ATOM | 189 | C | THR | | 29 | 43.641 | 100.024 | −73.198 | 1.00 | 17.65 | A | C |
| ATOM | 190 | O | THR | | 29 | 43.119 | 99.194 | −73.961 | 1.00 | 15.71 | A | O |
| ATOM | 191 | N | ASN | | 30 | 44.136 | 99.710 | −71.985 | 1.00 | 16.99 | A | N |
| ATOM | 192 | CA | ASN | | 30 | 44.211 | 98.339 | −71.437 | 1.00 | 16.73 | A | C |
| ATOM | 193 | CB | ASN | | 30 | 42.816 | 97.728 | −71.279 | 1.00 | 17.99 | A | C |
| ATOM | 194 | CG | ASN | | 30 | 42.089 | 98.314 | −70.063 | 1.00 | 20.60 | A | C |
| ATOM | 195 | OD1 | ASN | | 30 | 40.852 | 98.465 | −70.051 | 1.00 | 19.73 | A | O |
| ATOM | 196 | ND2 | ASN | | 30 | 42.867 | 98.662 | −69.049 | 1.00 | 19.70 | A | N |
| ATOM | 197 | C | ASN | | 30 | 45.153 | 97.439 | −72.264 | 1.00 | 15.22 | A | C |
| ATOM | 198 | O | ASN | | 30 | 45.102 | 96.197 | −72.183 | 1.00 | 15.45 | A | O |
| ATOM | 199 | N | ALA | | 31 | 46.037 | 98.063 | −73.036 | 1.00 | 16.39 | A | N |
| ATOM | 200 | CA | ALA | | 31 | 47.012 | 97.291 | −73.811 | 1.00 | 15.60 | A | C |
| ATOM | 201 | CB | ALA | | 31 | 47.695 | 98.166 | −74.885 | 1.00 | 14.03 | A | C |
| ATOM | 202 | C | ALA | | 31 | 48.104 | 96.792 | −72.875 | 1.00 | 18.33 | A | C |
| ATOM | 203 | O | ALA | | 31 | 48.447 | 97.468 | −71.875 | 1.00 | 16.84 | A | O |
| ATOM | 204 | N | SER | | 32 | 48.681 | 95.631 | −73.186 | 1.00 | 17.05 | A | N |
| ATOM | 205 | CA | SER | | 32 | 49.823 | 95.169 | −72.392 | 1.00 | 18.60 | A | C |
| ATOM | 206 | CB | SER | | 32 | 49.423 | 94.162 | −71.314 | 1.00 | 18.58 | A | C |
| ATOM | 207 | OG | SER | | 32 | 48.566 | 93.155 | −71.782 | 1.00 | 21.59 | A | O |
| ATOM | 208 | C | SER | | 32 | 50.866 | 94.576 | −73.343 | 1.00 | 19.74 | A | C |
| ATOM | 209 | O | SER | | 32 | 50.506 | 93.946 | −74.328 | 1.00 | 19.91 | A | O |
| ATOM | 210 | N | GLN | | 33 | 52.147 | 94.817 | −73.081 | 1.00 | 20.71 | A | N |
| ATOM | 211 | CA | GLN | | 33 | 53.188 | 94.286 | −73.953 | 1.00 | 21.40 | A | C |
| ATOM | 212 | CB | GLN | | 33 | 53.450 | 95.245 | −75.117 | 1.00 | 21.56 | A | C |
| ATOM | 213 | CG | GLN | | 33 | 53.840 | 96.646 | −74.684 | 1.00 | 26.07 | A | C |
| ATOM | 214 | CD | GLN | | 33 | 54.032 | 97.581 | −75.876 | 1.00 | 31.95 | A | C |
| ATOM | 215 | OE1 | GLN | | 33 | 54.935 | 98.444 | −75.899 | 1.00 | 32.77 | A | O |
| ATOM | 216 | NE2 | GLN | | 33 | 53.169 | 97.424 | −76.866 | 1.00 | 33.72 | A | N |
| ATOM | 217 | C | GLN | | 33 | 54.486 | 94.104 | −73.156 | 1.00 | 23.24 | A | C |
| ATOM | 218 | O | GLN | | 33 | 54.689 | 94.755 | −72.134 | 1.00 | 20.88 | A | O |
| ATOM | 219 | N | TRP | | 34 | 55.364 | 93.219 | −73.637 | 1.00 | 24.29 | A | N |
| ATOM | 220 | CA | TRP | | 34 | 56.668 | 93.021 | −72.963 | 1.00 | 23.41 | A | C |
| ATOM | 221 | CB | TRP | | 34 | 57.342 | 91.721 | −73.458 | 1.00 | 22.12 | A | C |
| ATOM | 222 | CG | TRP | | 34 | 56.627 | 90.467 | −73.042 | 1.00 | 19.46 | A | C |
| ATOM | 223 | CD2 | TRP | | 34 | 56.455 | 89.973 | −71.713 | 1.00 | 20.92 | A | C |
| ATOM | 224 | CE2 | TRP | | 34 | 55.689 | 88.808 | −71.807 | 1.00 | 20.79 | A | C |
| ATOM | 225 | CE3 | TRP | | 34 | 56.880 | 90.408 | −70.447 | 1.00 | 20.24 | A | C |
| ATOM | 226 | CD1 | TRP | | 34 | 55.984 | 89.602 | −73.851 | 1.00 | 19.94 | A | C |
| ATOM | 227 | NE1 | TRP | | 34 | 55.428 | 88.608 | −73.134 | 1.00 | 23.04 | A | N |
| ATOM | 228 | CZ2 | TRP | | 34 | 55.321 | 88.051 | −70.690 | 1.00 | 23.58 | A | C |
| ATOM | 229 | CZ3 | TRP | | 34 | 56.515 | 89.661 | −69.335 | 1.00 | 21.37 | A | C |
| ATOM | 230 | CH2 | TRP | | 34 | 55.743 | 88.497 | −69.462 | 1.00 | 22.61 | A | C |
| ATOM | 231 | C | TRP | | 34 | 57.599 | 94.201 | −73.261 | 1.00 | 26.89 | A | C |
| ATOM | 232 | O | TRP | | 34 | 58.287 | 94.706 | −72.391 | 1.00 | 24.04 | A | O |
| ATOM | 233 | N | GLU | | 35 | 57.604 | 94.633 | −74.514 | 1.00 | 30.77 | A | N |
| ATOM | 234 | CA | GLU | | 35 | 58.468 | 95.709 | −74.971 | 1.00 | 36.06 | A | C |
| ATOM | 235 | CB | GLU | | 35 | 58.374 | 95.790 | −76.506 | 1.00 | 39.46 | A | C |
| ATOM | 236 | CG | GLU | | 35 | 58.593 | 94.439 | −77.249 | 1.00 | 42.44 | A | C |
| ATOM | 237 | CD | GLU | | 35 | 57.397 | 93.493 | −77.249 | 1.00 | 44.78 | A | C |
| ATOM | 238 | OE1 | GLU | | 35 | 57.598 | 92.298 | −77.603 | 1.00 | 44.98 | A | O |
| ATOM | 239 | OE2 | GLU | | 35 | 56.267 | 93.932 | −76.902 | 1.00 | 42.57 | A | O |
| ATOM | 240 | C | GLU | | 35 | 58.179 | 97.065 | −74.349 | 1.00 | 38.85 | A | C |
| ATOM | 241 | O | GLU | | 35 | 57.020 | 97.420 | −74.105 | 1.00 | 38.53 | A | O |
| ATOM | 242 | N | ARG | | 36 | 59.225 | 97.828 | −74.065 | 1.00 | 41.91 | A | N |
| ATOM | 243 | CA | ARG | | 36 | 59.014 | 99.142 | −73.484 | 1.00 | 45.70 | A | C |
| ATOM | 244 | CB | ARG | | 36 | 60.337 | 99.764 | −73.045 | 1.00 | 47.04 | A | C |
| ATOM | 245 | CG | ARG | | 36 | 60.168 | 100.953 | −72.104 | 1.00 | 49.42 | A | C |
| ATOM | 246 | CD | ARG | | 36 | 61.453 | 101.188 | −71.299 | 1.00 | 52.01 | A | C |
| ATOM | 247 | NE | ARG | | 36 | 61.296 | 102.105 | −70.166 | 1.00 | 53.09 | A | N |
| ATOM | 248 | CZ | ARG | | 36 | 61.202 | 103.430 | −70.275 | 1.00 | 54.34 | A | C |
| ATOM | 249 | NH1 | ARG | | 36 | 61.248 | 104.014 | −71.474 | 1.00 | 54.73 | A | N |
| ATOM | 250 | NH2 | ARG | | 36 | 61.065 | 104.173 | −69.181 | 1.00 | 54.52 | A | N |
| ATOM | 251 | C | ARG | | 36 | 58.345 | 99.990 | −74.571 | 1.00 | 47.26 | A | C |
| ATOM | 252 | O | ARG | | 36 | 58.752 | 99.966 | −75.743 | 1.00 | 47.67 | A | O |
| ATOM | 253 | N | PRO | | 37 | 57.294 | 100.737 | −74.197 | 1.00 | 48.38 | A | N |
| ATOM | 254 | CD | PRO | | 37 | 56.760 | 100.921 | −72.833 | 1.00 | 47.75 | A | C |
| ATOM | 255 | CA | PRO | | 37 | 56.572 | 101.581 | −75.153 | 1.00 | 49.26 | A | C |
| ATOM | 256 | CB | PRO | | 37 | 55.342 | 102.005 | −74.366 | 1.00 | 48.18 | A | C |
| ATOM | 257 | CG | PRO | | 37 | 55.883 | 102.155 | −72.996 | 1.00 | 48.85 | A | C |
| ATOM | 258 | C | PRO | | 37 | 57.366 | 102.772 | −75.667 | 1.00 | 50.39 | A | C |
| ATOM | 259 | O | PRO | | 37 | 58.022 | 103.487 | −74.884 | 1.00 | 50.55 | A | O |
| ATOM | 260 | N | SER | | 38 | 57.270 | 102.974 | −76.985 | 1.00 | 51.87 | A | N |
| ATOM | 261 | CA | SER | | 38 | 57.940 | 104.057 | −77.714 | 1.00 | 53.51 | A | C |
| ATOM | 262 | CB | SER | | 38 | 58.390 | 103.559 | −79.097 | 1.00 | 54.83 | A | C |
| ATOM | 263 | OG | SER | | 38 | 57.285 | 103.149 | −79.901 | 1.00 | 55.33 | A | O |
| ATOM | 264 | C | SER | | 38 | 57.056 | 105.294 | −77.902 | 1.00 | 53.14 | A | C |
| ATOM | 265 | O | SER | | 38 | 55.911 | 105.336 | −77.452 | 1.00 | 52.91 | A | O |
| ATOM | 266 | N | GLU | | 51 | 49.209 | 115.419 | −78.922 | 1.00 | 57.18 | A | N |
| ATOM | 267 | CA | GLU | | 51 | 48.450 | 114.425 | −79.670 | 1.00 | 56.76 | A | C |
| ATOM | 268 | CB | GLU | | 51 | 47.325 | 113.852 | −78.800 | 1.00 | 57.06 | A | C |
| ATOM | 269 | CG | GLU | | 51 | 46.148 | 114.799 | −78.627 | 1.00 | 56.37 | A | C |
| ATOM | 270 | CD | GLU | | 51 | 45.028 | 114.202 | −77.794 | 1.00 | 55.93 | A | C |
| ATOM | 271 | OE1 | GLU | | 51 | 45.304 | 113.389 | −76.877 | 1.00 | 55.39 | A | O |
| ATOM | 272 | OE2 | GLU | | 51 | 43.865 | 114.566 | −78.041 | 1.00 | 55.53 | A | O |
| ATOM | 273 | C | GLU | | 51 | 47.828 | 115.043 | −80.920 | 1.00 | 56.21 | A | C |
| ATOM | 274 | O | GLU | | 51 | 47.691 | 116.264 | −81.024 | 1.00 | 56.71 | A | O |
| ATOM | 275 | N | PRO | | 52 | 47.426 | 114.193 | −81.885 | 1.00 | 55.50 | A | N |
| ATOM | 276 | CD | PRO | | 52 | 47.674 | 112.739 | −81.850 | 1.00 | 55.06 | A | C |
| ATOM | 277 | CA | PRO | | 52 | 46.799 | 114.593 | −83.159 | 1.00 | 53.57 | A | C |
| ATOM | 278 | CB | PRO | | 52 | 46.896 | 113.326 | −83.996 | 1.00 | 54.59 | A | C |
| ATOM | 279 | CG | PRO | | 52 | 46.753 | 112.233 | −82.936 | 1.00 | 55.33 | A | C |
| ATOM | 280 | C | PRO | | 52 | 45.343 | 115.073 | −83.006 | 1.00 | 51.62 | A | C |
| ATOM | 281 | O | PRO | | 52 | 44.654 | 114.703 | −82.048 | 1.00 | 52.15 | A | O |
| ATOM | 282 | N | ALA | | 53 | 44.893 | 115.889 | −83.958 | 1.00 | 48.32 | A | N |
| ATOM | 283 | CA | ALA | | 53 | 43.542 | 116.429 | −83.966 | 1.00 | 45.39 | A | C |
| ATOM | 284 | CB | ALA | | 53 | 43.419 | 117.505 | −85.043 | 1.00 | 46.60 | A | C |
| ATOM | 285 | C | ALA | | 53 | 42.533 | 115.325 | −84.233 | 1.00 | 42.66 | A | C |
| ATOM | 286 | O | ALA | | 53 | 41.390 | 115.358 | −83.750 | 1.00 | 40.93 | A | O |
| ATOM | 287 | N | ARG | | 54 | 42.939 | 114.351 | −85.040 | 1.00 | 40.42 | A | N |
| ATOM | 288 | CA | ARG | | 54 | 42.045 | 113.246 | −85.349 | 1.00 | 36.45 | A | C |
| ATOM | 289 | CB | ARG | | 54 | 41.251 | 113.519 | −86.630 | 1.00 | 40.21 | A | C |
| ATOM | 290 | CG | ARG | | 54 | 40.899 | 114.989 | −86.886 | 1.00 | 44.89 | A | C |
| ATOM | 291 | CD | ARG | | 54 | 40.238 | 115.137 | −88.252 | 1.00 | 47.53 | A | C |
| ATOM | 292 | NE | ARG | | 54 | 38.839 | 114.737 | −88.196 | 1.00 | 50.86 | A | N |
| ATOM | 293 | CZ | ARG | | 54 | 38.175 | 114.214 | −89.222 | 1.00 | 53.11 | A | C |
| ATOM | 294 | NH1 | ARG | | 54 | 38.798 | 114.023 | −90.383 | 1.00 | 53.57 | A | N |
| ATOM | 295 | NH2 | ARG | | 54 | 36.882 | 113.902 | −89.095 | 1.00 | 54.45 | A | N |
| ATOM | 296 | C | ARG | | 54 | 42.830 | 111.958 | −85.522 | 1.00 | 32.29 | A | C |
| ATOM | 297 | O | ARG | | 54 | 44.053 | 111.953 | −85.739 | 1.00 | 31.13 | A | O |
| ATOM | 298 | N | VAL | | 55 | 42.107 | 110.865 | −85.383 | 1.00 | 27.12 | A | N |
| ATOM | 299 | CA | VAL | | 55 | 42.686 | 109.548 | −85.567 | 1.00 | 24.51 | A | C |
| ATOM | 300 | CB | VAL | | 55 | 42.991 | 108.855 | −84.192 | 1.00 | 22.55 | A | C |
| ATOM | 301 | CG1 | VAL | | 55 | 43.986 | 109.705 | −83.379 | 1.00 | 21.99 | A | C |
| ATOM | 302 | CG2 | VAL | | 55 | 41.718 | 108.674 | −83.399 | 1.00 | 21.28 | A | C |
| ATOM | 303 | C | VAL | | 55 | 41.622 | 108.746 | −86.314 | 1.00 | 23.57 | A | C |
| ATOM | 304 | O | VAL | | 55 | 40.447 | 109.142 | −86.374 | 1.00 | 25.42 | A | O |
| ATOM | 305 | N | ARG | | 56 | 42.046 | 107.653 | −86.922 | 1.00 | 20.61 | A | N |
| ATOM | 306 | CA | ARG | | 56 | 41.126 | 106.781 | −87.585 | 1.00 | 19.73 | A | C |
| ATOM | 307 | CB | ARG | | 56 | 41.509 | 106.633 | −89.052 | 1.00 | 21.17 | A | C |
| ATOM | 308 | CG | ARG | | 56 | 40.532 | 105.747 | −89.800 | 1.00 | 21.29 | A | C |
| ATOM | 309 | CD | ARG | | 56 | 40.922 | 105.617 | −91.285 | 1.00 | 23.33 | A | C |
| ATOM | 310 | NE | ARG | | 56 | 39.929 | 104.837 | −92.041 | 1.00 | 24.93 | A | N |
| ATOM | 311 | CZ | ARG | | 56 | 40.074 | 104.542 | −93.331 | 1.00 | 28.00 | A | C |
| ATOM | 312 | NH1 | ARG | | 56 | 41.171 | 104.967 | −93.974 | 1.00 | 27.95 | A | N |
| ATOM | 313 | NH2 | ARG | | 56 | 39.131 | 103.866 | −93.983 | 1.00 | 26.57 | A | N |
| ATOM | 314 | C | ARG | | 56 | 41.248 | 105.433 | −86.864 | 1.00 | 19.23 | A | C |
| ATOM | 315 | O | ARG | | 56 | 42.354 | 104.988 | −86.591 | 1.00 | 18.91 | A | O |
| ATOM | 316 | N | CYS | | 57 | 40.125 | 104.796 | −86.545 | 1.00 | 18.32 | A | N |
| ATOM | 317 | CA | CYS | | 57 | 40.167 | 103.512 | −85.866 | 1.00 | 18.91 | A | C |
| ATOM | 318 | CB | CYS | | 57 | 39.850 | 103.691 | −84.362 | 1.00 | 17.51 | A | C |
| ATOM | 319 | SG | CYS | | 57 | 41.115 | 104.631 | −83.467 | 1.00 | 19.22 | A | S |
| ATOM | 320 | C | CYS | | 57 | 39.173 | 102.555 | −86.429 | 1.00 | 18.65 | A | C |
| ATOM | 321 | O | CYS | | 57 | 38.209 | 102.973 | −87.078 | 1.00 | 18.74 | A | O |
| ATOM | 322 | N | SER | | 58 | 39.449 | 101.264 | −86.230 | 1.00 | 15.34 | A | N |
| ATOM | 323 | CA | SER | | 58 | 38.516 | 100.212 | −86.510 | 1.00 | 14.85 | A | C |
| ATOM | 324 | CB | SER | | 58 | 39.100 | 99.128 | −87.415 | 1.00 | 15.27 | A | C |
| ATOM | 325 | OG | SER | | 58 | 39.344 | 99.696 | −88.683 | 1.00 | 16.58 | A | O |
| ATOM | 326 | C | SER | | 58 | 38.284 | 99.619 | −85.102 | 1.00 | 16.36 | A | C |
| ATOM | 327 | O | SER | | 58 | 39.099 | 99.885 | −84.168 | 1.00 | 16.10 | A | O |
| ATOM | 328 | N | HIS | | 59 | 37.208 | 98.830 | −84.944 | 1.00 | 14.27 | A | N |
| ATOM | 329 | CA | HIS | | 59 | 36.911 | 98.178 | −83.684 | 1.00 | 16.12 | A | C |
| ATOM | 330 | CB | HIS | | 59 | 36.137 | 99.111 | −82.735 | 1.00 | 17.98 | A | C |
| ATOM | 331 | CG | HIS | | 59 | 34.692 | 99.304 | −83.109 | 1.00 | 19.80 | A | C |
| ATOM | 332 | CD2 | HIS | | 59 | 34.091 | 99.490 | −84.312 | 1.00 | 20.67 | A | C |
| ATOM | 333 | ND1 | HIS | | 59 | 33.677 | 99.307 | −82.172 | 1.00 | 22.45 | A | N |
| ATOM | 334 | CE1 | HIS | | 59 | 32.516 | 99.484 | −82.784 | 1.00 | 23.18 | A | C |
| ATOM | 335 | NE2 | HIS | | 59 | 32.736 | 99.598 | −84.082 | 1.00 | 21.45 | A | N |
| ATOM | 336 | C | HIS | | 59 | 36.135 | 96.894 | −83.853 | 1.00 | 15.59 | A | C |
| ATOM | 337 | O | HIS | | 59 | 35.561 | 96.613 | −84.911 | 1.00 | 16.30 | A | O |
| ATOM | 338 | N | LEU | | 60 | 36.123 | 96.096 | −82.795 | 1.00 | 15.71 | A | N |
| ATOM | 339 | CA | LEU | | 60 | 35.361 | 94.877 | −82.785 | 1.00 | 15.37 | A | C |
| ATOM | 340 | CB | LEU | | 60 | 36.272 | 93.650 | −82.764 | 1.00 | 14.76 | A | C |
| ATOM | 341 | CG | LEU | | 60 | 35.571 | 92.283 | −82.913 | 1.00 | 14.78 | A | C |
| ATOM | 342 | CD1 | LEU | | 60 | 36.665 | 91.248 | −83.255 | 1.00 | 15.94 | A | C |
| ATOM | 343 | CD2 | LEU | | 60 | 34.776 | 91.858 | −81.625 | 1.00 | 14.00 | A | C |
| ATOM | 344 | C | LEU | | 60 | 34.606 | 95.015 | −81.466 | 1.00 | 16.89 | A | C |
| ATOM | 345 | O | LEU | | 60 | 35.196 | 95.105 | −80.375 | 1.00 | 16.94 | A | O |
| ATOM | 346 | N | LEU | | 61 | 33.294 | 95.026 | −81.561 | 1.00 | 16.34 | A | N |
| ATOM | 347 | CA | LEU | | 61 | 32.443 | 95.195 | −80.389 | 1.00 | 17.70 | A | C |
| ATOM | 348 | CB | LEU | | 61 | 31.347 | 96.237 | −80.726 | 1.00 | 19.58 | A | C |
| ATOM | 349 | CG | LEU | | 61 | 30.275 | 96.422 | −79.621 | 1.00 | 20.38 | A | C |
| ATOM | 350 | CD1 | LEU | | 61 | 30.927 | 96.933 | −78.351 | 1.00 | 21.58 | A | C |
| ATOM | 351 | CD2 | LEU | | 61 | 29.189 | 97.410 | −80.084 | 1.00 | 22.24 | A | C |
| ATOM | 352 | C | LEU | | 61 | 31.766 | 93.888 | −80.011 | 1.00 | 16.77 | A | C |
| ATOM | 353 | O | LEU | | 61 | 31.257 | 93.188 | −80.883 | 1.00 | 16.83 | A | O |
| ATOM | 354 | N | VAL | | 62 | 31.796 | 93.534 | −78.719 | 1.00 | 16.21 | A | N |
| ATOM | 355 | CA | VAL | | 62 | 31.072 | 92.369 | −78.280 | 1.00 | 16.86 | A | C |
| ATOM | 356 | CB | VAL | | 62 | 31.958 | 91.257 | −77.666 | 1.00 | 18.07 | A | C |
| ATOM | 357 | CG1 | VAL | | 62 | 31.043 | 90.159 | −77.040 | 1.00 | 19.04 | A | C |
| ATOM | 358 | CG2 | VAL | | 62 | 32.853 | 90.637 | −78.760 | 1.00 | 17.25 | A | C |
| ATOM | 359 | C | VAL | | 62 | 30.108 | 92.918 | −77.219 | 1.00 | 19.42 | A | C |
| ATOM | 360 | O | VAL | | 62 | 30.517 | 93.485 | −76.192 | 1.00 | 17.73 | A | O |
| ATOM | 361 | N | LYS | | 63 | 28.822 | 92.798 | −77.528 | 1.00 | 20.54 | A | N |
| ATOM | 362 | CA | LYS | | 63 | 27.754 | 93.288 | −76.656 | 1.00 | 23.25 | A | C |
| ATOM | 363 | CB | LYS | | 63 | 26.504 | 93.624 | −77.509 | 1.00 | 23.31 | A | C |
| ATOM | 364 | CG | LYS | | 63 | 26.658 | 94.923 | −78.317 | 1.00 | 25.42 | A | C |
| ATOM | 365 | CD | LYS | | 63 | 25.334 | 95.370 | −79.028 | 1.00 | 24.98 | A | C |
| ATOM | 366 | CE | LYS | | 63 | 25.397 | 96.825 | −79.373 | 1.00 | 25.17 | A | C |
| ATOM | 367 | NZ | LYS | | 63 | 24.153 | 97.364 | −79.994 | 1.00 | 26.49 | A | N |
| ATOM | 368 | C | LYS | | 63 | 27.405 | 92.240 | −75.616 | 1.00 | 23.80 | A | C |
| ATOM | 369 | O | LYS | | 63 | 27.785 | 91.081 | −75.742 | 1.00 | 22.62 | A | O |
| ATOM | 370 | N | HIS | | 64 | 26.722 | 92.666 | −74.555 | 1.00 | 25.65 | A | N |
| ATOM | 371 | CA | HIS | | 64 | 26.285 | 91.744 | −73.526 | 1.00 | 27.03 | A | C |
| ATOM | 372 | CE | HIS | | 64 | 27.299 | 91.653 | −72.379 | 1.00 | 25.01 | A | C |
| ATOM | 373 | CG | HIS | | 64 | 27.759 | 92.979 | −71.877 | 1.00 | 27.44 | A | C |
| ATOM | 374 | CD2 | HIS | | 64 | 28.914 | 93.656 | −72.091 | 1.00 | 27.72 | A | C |
| ATOM | 375 | ND1 | HIS | | 64 | 26.968 | 93.802 | −71.107 | 1.00 | 27.68 | A | N |
| ATOM | 376 | CE1 | HIS | | 64 | 27.615 | 94.930 | −70.869 | 1.00 | 29.90 | A | C |
| ATOM | 377 | NE2 | HIS | | 64 | 28.799 | 94.865 | −71.457 | 1.00 | 29.44 | A | N |
| ATOM | 378 | C | HIS | | 64 | 24.918 | 92.210 | −73.016 | 1.00 | 28.64 | A | C |
| ATOM | 379 | O | HIS | | 64 | 24.411 | 93.265 | −73.428 | 1.00 | 28.23 | A | O |
| ATOM | 380 | N | SER | | 65 | 24.332 | 91.421 | −72.122 | 1.00 | 31.14 | A | N |
| ATOM | 381 | CA | SER | | 65 | 22.991 | 91.720 | −71.594 | 1.00 | 32.46 | A | C |
| ATOM | 382 | CB | SER | | 65 | 22.559 | 90.610 | −70.630 | 1.00 | 32.73 | A | C |
| ATOM | 383 | OG | SER | | 65 | 23.329 | 90.701 | −69.454 | 1.00 | 34.51 | A | O |
| ATOM | 384 | C | SER | | 65 | 22.834 | 93.092 | −70.930 | 1.00 | 33.00 | A | C |
| ATOM | 385 | O | SER | | 65 | 21.715 | 93.600 | −70.819 | 1.00 | 34.17 | A | O |
| ATOM | 386 | N | GLN | | 66 | 23.917 | 93.709 | −70.477 | 1.00 | 31.75 | A | N |
| ATOM | 387 | CA | GLN | | 66 | 23.774 | 95.038 | −69.890 | 1.00 | 32.98 | A | C |
| ATOM | 388 | CB | GLN | | 66 | 24.635 | 95.212 | −68.629 | 1.00 | 36.13 | A | C |
| ATOM | 389 | CG | GLN | | 66 | 24.134 | 94.486 | −67.372 | 1.00 | 40.19 | A | C |
| ATOM | 390 | CD | GLN | | 66 | 24.997 | 94.835 | −66.128 | 1.00 | 44.56 | A | C |
| ATOM | 391 | OE1 | GLN | | 66 | 24.915 | 95.956 | −65.582 | 1.00 | 44.70 | A | O |
| ATOM | 392 | NE2 | GLN | | 66 | 25.842 | 93.886 | −65.705 | 1.00 | 44.23 | A | N |
| ATOM | 393 | C | GLN | | 66 | 24.132 | 96.153 | −70.872 | 1.00 | 32.44 | A | C |
| ATOM | 394 | O | GLN | | 66 | 24.233 | 97.309 | −70.469 | 1.00 | 34.82 | A | O |
| ATOM | 395 | N | SER | | 67 | 24.351 | 95.827 | −72.148 | 1.00 | 31.99 | A | N |
| ATOM | 396 | CA | SER | | 67 | 24.662 | 96.868 | −73.136 | 1.00 | 32.31 | A | C |
| ATOM | 397 | CB | SER | | 67 | 24.995 | 96.253 | −74.494 | 1.00 | 32.03 | A | C |
| ATOM | 398 | OG | SER | | 67 | 26.245 | 95.613 | −74.475 | 1.00 | 29.96 | A | O |
| ATOM | 399 | C | SER | | 67 | 23.449 | 97.776 | −73.314 | 1.00 | 33.99 | A | C |
| ATOM | 400 | O | SER | | 67 | 22.316 | 97.291 | −73.323 | 1.00 | 33.76 | A | O |
| ATOM | 401 | N | ARG | | 68 | 23.697 | 99.071 | −73.495 | 1.00 | 35.83 | A | N |
| ATOM | 402 | CA | ARG | | 68 | 22.616 | 100.037 | −73.676 | 1.00 | 39.36 | A | C |
| ATOM | 403 | CB | ARG | | 68 | 23.166 | 101.348 | −74.236 | 1.00 | 40.70 | A | C |
| ATOM | 404 | CG | ARG | | 68 | 22.250 | 102.585 | −74.079 | 1.00 | 44.65 | A | C |
| ATOM | 405 | CD | ARG | | 68 | 21.685 | 102.775 | −72.658 | 1.00 | 46.83 | A | C |
| ATOM | 406 | NE | ARG | | 68 | 22.693 | 102.918 | −71.601 | 1.00 | 49.24 | A | N |
| ATOM | 407 | CZ | ARG | | 68 | 22.393 | 102.960 | −70.298 | 1.00 | 50.09 | A | C |
| ATOM | 408 | NE1 | ARG | | 68 | 21.120 | 102.874 | −69.914 | 1.00 | 50.48 | A | N |
| ATOM | 409 | NH2 | ARG | | 68 | 23.349 | 103.067 | −69.376 | 1.00 | 48.03 | A | N |
| ATOM | 410 | C | ARG | | 68 | 21.566 | 99.444 | −74.611 | 1.00 | 41.07 | A | C |
| ATOM | 411 | O | ARG | | 68 | 20.357 | 99.579 | −74.365 | 1.00 | 42.13 | A | O |
| ATOM | 412 | N | ARG | | 69 | 22.011 | 98.766 | −75.672 | 1.00 | 40.46 | A | N |
| ATOM | 413 | CA | ARG | | 69 | 21.079 | 98.134 | −76.607 | 1.00 | 39.68 | A | C |
| ATOM | 414 | CB | ARG | | 69 | 21.008 | 98.959 | −77.896 | 1.00 | 42.24 | A | C |
| ATOM | 415 | CG | ARG | | 69 | 19.924 | 98.496 | −78.850 | 1.00 | 46.49 | A | C |
| ATOM | 416 | CD | ARG | | 69 | 20.034 | 99.176 | −80.218 | 1.00 | 49.68 | A | C |
| ATOM | 417 | NE | ARG | | 69 | 19.828 | 98.223 | −81.321 | 1.00 | 52.37 | A | N |
| ATOM | 418 | CZ | ARG | | 69 | 20.755 | 97.921 | −82.236 | 1.00 | 53.19 | A | C |
| ATOM | 419 | NH1 | ARG | | 69 | 21.954 | 98.504 | −82.179 | 1.00 | 52.88 | A | N |
| ATOM | 420 | NH2 | ARG | | 69 | 20.493 | 97.037 | −83.205 | 1.00 | 53.10 | A | N |
| ATOM | 421 | C | ARG | | 69 | 21.507 | 96.678 | −76.897 | 1.00 | 37.70 | A | C |
| ATOM | 422 | O | ARG | | 69 | 22.292 | 96.415 | −77.808 | 1.00 | 36.77 | A | O |
| ATOM | 423 | N | PRO | | 70 | 20.993 | 95.713 | −76.111 | 1.00 | 36.30 | A | N |
| ATOM | 424 | CD | PRO | | 70 | 20.097 | 95.941 | −74.966 | 1.00 | 36.12 | A | C |
| ATOM | 425 | CA | PRO | | 70 | 21.304 | 94.279 | −76.246 | 1.00 | 35.56 | A | C |
| ATOM | 426 | CB | PRO | | 70 | 20.672 | 93.674 | −74.988 | 1.00 | 36.73 | A | C |
| ATOM | 427 | CG | PRO | | 70 | 19.520 | 94.578 | −74.736 | 1.00 | 35.81 | A | C |
| ATOM | 428 | C | PRO | | 70 | 20.803 | 93.633 | −77.536 | 1.00 | 34.10 | A | C |
| ATOM | 429 | O | PRO | | 70 | 20.082 | 92.630 | −77.520 | 1.00 | 32.97 | A | O |
| ATOM | 430 | N | SER | | 71 | 21.237 | 94.185 | −78.665 | 1.00 | 31.96 | A | N |
| ATOM | 431 | CA | SER | | 71 | 20.791 | 93.679 | −79.953 | 1.00 | 30.21 | A | C |
| ATOM | 432 | CB | SER | | 71 | 19.421 | 94.306 | −80.256 | 1.00 | 30.35 | A | C |
| ATOM | 433 | OG | SER | | 71 | 19.105 | 94.177 | −81.615 | 1.00 | 34.54 | A | O |
| ATOM | 434 | C | SER | | 71 | 21.792 | 94.081 | −81.024 | 1.00 | 28.83 | A | C |
| ATOM | 435 | O | SER | | 71 | 22.439 | 95.108 | −80.872 | 1.00 | 30.56 | A | O |
| ATOM | 436 | N | SER | | 72 | 21.942 | 93.271 | −82.070 | 1.00 | 28.37 | A | N |
| ATOM | 437 | CA | SER | | 72 | 22.866 | 93.593 | −83.183 | 1.00 | 27.81 | A | C |
| ATOM | 438 | CB | SER | | 72 | 24.324 | 93.238 | −82.826 | 1.00 | 26.47 | A | C |
| ATOM | 439 | OG | SER | | 72 | 24.548 | 91.845 | −82.900 | 1.00 | 23.77 | A | O |
| ATOM | 440 | C | SER | | 72 | 22.483 | 92.824 | −84.431 | 1.00 | 27.70 | A | C |
| ATOM | 441 | O | SER | | 72 | 21.604 | 91.963 | −84.386 | 1.00 | 26.94 | A | O |
| ATOM | 442 | N | TRP | | 73 | 23.170 | 93.119 | −85.539 | 1.00 | 27.27 | A | N |
| ATOM | 443 | CA | TRP | | 73 | 22.921 | 92.441 | −86.786 | 1.00 | 26.26 | A | C |
| ATOM | 444 | CB | TRP | | 73 | 23.792 | 93.007 | −87.920 | 1.00 | 27.78 | A | C |
| ATOM | 445 | CG | TRP | | 73 | 25.310 | 92.698 | −87.796 | 1.00 | 27.28 | A | C |
| ATOM | 446 | CD2 | TRP | | 73 | 26.000 | 91.521 | −88.260 | 1.00 | 28.37 | A | C |
| ATOM | 447 | CE2 | TRP | | 73 | 27.361 | 91.657 | −87.890 | 1.00 | 27.09 | A | C |
| ATOM | 448 | CE3 | TRP | | 73 | 25.603 | 90.360 | −88.946 | 1.00 | 28.76 | A | C |
| ATOM | 449 | CD1 | TRP | | 73 | 26.264 | 93.482 | −87.182 | 1.00 | 26.53 | A | C |
| ATOM | 450 | NE1 | TRP | | 73 | 27.492 | 92.854 | −87.236 | 1.00 | 26.50 | A | N |
| ATOM | 451 | CZ2 | TRP | | 73 | 28.323 | 90.673 | −88.185 | 1.00 | 27.92 | A | C |
| ATOM | 452 | CZ3 | TRP | | 73 | 26.568 | 89.389 | −89.234 | 1.00 | 29.51 | A | C |
| ATOM | 453 | CH2 | TRP | | 73 | 27.909 | 89.557 | −88.850 | 1.00 | 28.96 | A | C |
| ATOM | 454 | C | TRP | | 73 | 23.219 | 90.966 | −86.623 | 1.00 | 25.59 | A | C |
| ATOM | 455 | O | TRP | | 73 | 22.769 | 90.178 | −87.422 | 1.00 | 25.81 | A | O |
| ATOM | 456 | N | ARG | | 74 | 23.984 | 90.585 | −85.591 | 1.00 | 25.58 | A | N |
| ATOM | 457 | CA | ARG | | 74 | 24.318 | 89.186 | −85.423 | 1.00 | 25.70 | A | C |
| ATOM | 458 | CB | ARG | | 74 | 25.641 | 88.974 | −84.657 | 1.00 | 25.07 | A | C |
| ATOM | 459 | CG | ARG | | 74 | 26.844 | 89.705 | −85.242 | 1.00 | 25.70 | A | C |
| ATOM | 460 | CD | ARG | | 74 | 28.117 | 89.182 | −84.542 | 1.00 | 23.97 | A | C |
| ATOM | 461 | NE | ARG | | 74 | 28.219 | 87.793 | −84.902 | 1.00 | 24.15 | A | N |
| ATOM | 462 | CZ | ARG | | 74 | 28.577 | 86.800 | −84.110 | 1.00 | 27.45 | A | C |
| ATOM | 463 | NH1 | ARG | | 74 | 28.893 | 87.016 | −82.841 | 1.00 | 29.21 | A | N |
| ATOM | 464 | NH2 | ARG | | 74 | 28.628 | 85.568 | −84.623 | 1.00 | 29.07 | A | N |
| ATOM | 465 | C | ARG | | 74 | 23.241 | 88.432 | −84.693 | 1.00 | 26.35 | A | C |
| ATOM | 466 | O | ARG | | 74 | 23.081 | 87.250 | −84.916 | 1.00 | 25.66 | A | O |
| ATOM | 467 | N | GLN | | 75 | 22.555 | 89.092 | −83.773 | 1.00 | 27.95 | A | N |
| ATOM | 468 | CA | GLN | | 75 | 21.472 | 88.424 | −83.071 | 1.00 | 30.67 | A | C |
| ATOM | 469 | CB | GLN | | 75 | 21.968 | 87.406 | −82.041 | 1.00 | 33.29 | A | C |
| ATOM | 470 | CG | GLN | | 75 | 22.933 | 87.909 | −81.045 | 1.00 | 38.39 | A | C |
| ATOM | 471 | CD | GLN | | 75 | 23.171 | 86.916 | −79.906 | 1.00 | 40.83 | A | C |
| ATOM | 472 | OE1 | GLN | | 75 | 24.094 | 87.084 | −79.123 | 1.00 | 42.06 | A | O |
| ATOM | 473 | NE2 | GLN | | 75 | 22.325 | 85.888 | −79.807 | 1.00 | 41.95 | A | N |
| ATOM | 474 | C | GLN | | 75 | 20.555 | 89.444 | −82.436 | 1.00 | 30.27 | A | C |
| ATOM | 475 | O | GLN | | 75 | 20.999 | 90.384 | −81.764 | 1.00 | 29.85 | A | O |
| ATOM | 476 | N | GLU | | 76 | 19.265 | 89.221 | −82.673 | 1.00 | 28.72 | A | N |
| ATOM | 477 | CA | GLU | | 76 | 18.215 | 90.112 | −82.243 | 1.00 | 27.36 | A | C |
| ATOM | 478 | CB | GLU | | 76 | 16.863 | 89.594 | −82.742 | 1.00 | 27.38 | A | C |
| ATOM | 479 | CG | GLU | | 76 | 15.697 | 90.576 | −82.466 | 1.00 | 29.30 | A | C |
| ATOM | 480 | CD | GLU | | 76 | 14.499 | 90.336 | −83.397 | 1.00 | 28.66 | A | C |
| ATOM | 481 | OE1 | GLU | | 76 | 13.651 | 91.256 | −83.523 | 1.00 | 30.39 | A | O |
| ATOM | 482 | OE2 | GLU | | 76 | 14.422 | 89.240 | −83.994 | 1.00 | 28.30 | A | O |
| ATOM | 483 | C | GLU | | 76 | 18.175 | 90.389 | −80.759 | 1.00 | 28.25 | A | C |
| ATOM | 484 | O | GLU | | 76 | 18.032 | 91.555 | −80.357 | 1.00 | 27.45 | A | O |
| ATOM | 485 | N | LYS | | 77 | 18.293 | 89.345 | −79.947 | 1.00 | 28.62 | A | N |
| ATOM | 486 | CA | LYS | | 77 | 18.301 | 89.514 | −78.495 | 1.00 | 31.45 | A | C |
| ATOM | 487 | CB | LYS | | 77 | 17.206 | 88.641 | −77.864 | 1.00 | 33.56 | A | C |
| ATOM | 488 | CG | LYS | | 77 | 17.213 | 88.609 | −76.334 | 1.00 | 37.19 | A | C |
| ATOM | 489 | CD | LYS | | 77 | 16.092 | 87.688 | −75.823 | 1.00 | 39.33 | A | C |
| ATOM | 490 | CE | LYS | | 77 | 15.841 | 87.863 | −74.322 | 1.00 | 40.79 | A | C |
| ATOM | 491 | NZ | LYS | | 77 | 15.635 | 89.295 | −73.928 | 1.00 | 42.89 | A | N |
| ATOM | 492 | C | LYS | | 77 | 19.677 | 89.041 | −77.980 | 1.00 | 31.79 | A | C |
| ATOM | 493 | O | LYS | | 77 | 19.943 | 87.841 | −77.975 | 1.00 | 31.80 | A | O |
| ATOM | 494 | N | ILE | | 78 | 20.539 | 89.959 | −77.558 | 1.00 | 32.18 | A | N |
| ATOM | 495 | CA | ILE | | 78 | 21.858 | 89.546 | −77.044 | 1.00 | 32.83 | A | C |
| ATOM | 496 | CB | ILE | | 78 | 22.887 | 90.688 | −77.086 | 1.00 | 32.42 | A | C |
| ATOM | 497 | CG2 | ILE | | 78 | 24.184 | 90.235 | −76.374 | 1.00 | 31.44 | A | C |
| ATOM | 498 | CG1 | ILE | | 78 | 23.112 | 91.176 | −78.525 | 1.00 | 33.60 | A | C |
| ATOM | 499 | CD1 | ILE | | 78 | 24.010 | 90.342 | −79.327 | 1.00 | 34.34 | A | C |
| ATOM | 500 | C | ILE | | 78 | 21.652 | 89.189 | −75.566 | 1.00 | 32.54 | A | C |
| ATOM | 501 | O | ILE | | 78 | 21.245 | 90.046 | −74.780 | 1.00 | 33.58 | A | O |
| ATOM | 502 | N | THR | | 79 | 21.987 | 87.963 | −75.195 | 1.00 | 32.18 | A | N |
| ATOM | 503 | CA | THR | | 79 | 21.824 | 87.479 | −73.830 | 1.00 | 33.05 | A | C |
| ATOM | 504 | CB | THR | | 79 | 20.977 | 86.194 | −73.852 | 1.00 | 34.01 | A | C |
| ATOM | 505 | OG1 | THR | | 79 | 21.623 | 85.218 | −74.670 | 1.00 | 33.99 | A | O |
| ATOM | 506 | CG2 | THR | | 79 | 19.597 | 86.473 | −74.425 | 1.00 | 35.01 | A | C |
| ATOM | 507 | C | THR | | 79 | 23.104 | 87.157 | −73.032 | 1.00 | 33.80 | A | C |
| ATOM | 508 | O | THR | | 79 | 23.053 | 86.984 | −71.806 | 1.00 | 32.58 | A | O |
| ATOM | 509 | N | ARG | | 80 | 24.249 | 87.034 | −73.709 | 1.00 | 32.26 | A | N |
| ATOM | 510 | CA | ARG | | 80 | 25.488 | 86.718 | −72.991 | 1.00 | 29.63 | A | C |
| ATOM | 511 | CB | ARG | | 80 | 26.674 | 86.693 | −73.974 | 1.00 | 28.73 | A | C |
| ATOM | 512 | CG | ARG | | 80 | 27.100 | 88.081 | −74.521 | 1.00 | 27.80 | A | C |
| ATOM | 513 | CD | ARG | | 80 | 28.417 | 87.939 | −75.327 | 1.00 | 23.69 | A | C |
| ATOM | 514 | NE | ARG | | 80 | 28.204 | 87.284 | −76.633 | 1.00 | 21.30 | A | N |
| ATOM | 515 | CZ | ARG | | 80 | 27.807 | 87.918 | −77.743 | 1.00 | 20.50 | A | C |
| ATOM | 516 | NH1 | ARG | | 80 | 27.579 | 89.219 | −77.725 | 1.00 | 22.26 | A | N |
| ATOM | 517 | NH2 | ARG | | 80 | 27.639 | 87.243 | −78.888 | 1.00 | 24.11 | A | N |
| ATOM | 518 | C | ARG | | 80 | 25.762 | 87.706 | −71.866 | 1.00 | 28.69 | A | C |
| ATOM | 519 | O | ARG | | 80 | 25.391 | 88.882 | −71.942 | 1.00 | 29.22 | A | O |
| ATOM | 520 | N | THR | | 81 | 26.413 | 87.232 | −70.809 | 1.00 | 28.17 | A | N |
| ATOM | 521 | CA | THR | | 81 | 26.770 | 88.109 | −69.707 | 1.00 | 28.43 | A | C |
| ATOM | 522 | CB | THR | | 81 | 27.127 | 87.330 | −68.431 | 1.00 | 29.64 | A | C |
| ATOM | 523 | OG1 | THR | | 81 | 28.303 | 86.528 | −68.658 | 1.00 | 28.86 | A | O |
| ATOM | 524 | CG2 | THR | | 81 | 25.949 | 86.435 | −68.024 | 1.00 | 31.24 | A | C |
| ATOM | 525 | C | THR | | 81 | 27.995 | 88.947 | −70.064 | 1.00 | 27.38 | A | C |
| ATOM | 526 | O | THR | | 81 | 28.707 | 88.654 | −71.026 | 1.00 | 24.99 | A | O |
| ATOM | 527 | N | LYS | | 82 | 28.208 | 89.993 | −69.292 | 1.00 | 26.25 | A | N |
| ATOM | 528 | CA | LYS | | 82 | 29.358 | 90.869 | −69.474 | 1.00 | 28.43 | A | C |
| ATOM | 529 | CB | LYS | | 82 | 29.328 | 91.998 | −68.445 | 1.00 | 30.62 | A | C |
| ATOM | 530 | CG | LYS | | 82 | 30.500 | 92.969 | −68.552 | 1.00 | 36.28 | A | C |
| ATOM | 531 | CD | LYS | | 82 | 30.663 | 93.851 | −67.293 | 1.00 | 40.60 | A | C |
| ATOM | 532 | CE | LYS | | 82 | 29.409 | 94.672 | −67.011 | 1.00 | 43.01 | A | C |
| ATOM | 533 | NZ | LYS | | 82 | 29.540 | 95.526 | −65.796 | 1.00 | 44.43 | A | N |
| ATOM | 534 | C | LYS | | 82 | 30.646 | 90.033 | −69.308 | 1.00 | 28.17 | A | C |
| ATOM | 535 | O | LYS | | 82 | 31.633 | 90.226 | −70.020 | 1.00 | 25.71 | A | O |
| ATOM | 536 | N | GLU | | 83 | 30.624 | 89.076 | −68.384 | 1.00 | 26.83 | A | N |
| ATOM | 537 | CA | GLU | | 83 | 31.792 | 88.237 | −68.187 | 1.00 | 27.51 | A | C |
| ATOM | 538 | CB | GLU | | 83 | 31.624 | 87.370 | −66.923 | 1.00 | 30.84 | A | C |
| ATOM | 539 | CG | GLU | | 83 | 31.553 | 88.171 | −65.639 | 1.00 | 36.69 | A | C |
| ATOM | 540 | CD | GLU | | 83 | 30.277 | 89.053 | −65.493 | 1.00 | 41.12 | A | C |
| ATOM | 541 | OE1 | GLU | | 83 | 30.343 | 90.022 | −64.697 | 1.00 | 43.82 | A | O |
| ATOM | 542 | OE2 | GLU | | 83 | 29.223 | 88.789 | −66.138 | 1.00 | 40.61 | A | O |
| ATOM | 543 | C | GLU | | 83 | 32.081 | 87.335 | −69.387 | 1.00 | 25.09 | A | C |
| ATOM | 544 | O | GLU | | 83 | 33.240 | 87.120 | −69.728 | 1.00 | 22.09 | A | O |
| ATOM | 545 | N | GLU | | 84 | 31.038 | 86.800 | −70.024 | 1.00 | 23.73 | A | N |
| ATOM | 546 | CA | GLU | | 84 | 31.190 | 85.919 | −71.181 | 1.00 | 22.95 | A | C |
| ATOM | 547 | CB | GLU | | 84 | 29.848 | 85.284 | −71.553 | 1.00 | 23.71 | A | C |
| ATOM | 548 | CG | GLU | | 84 | 29.357 | 84.217 | −70.591 | 1.00 | 29.98 | A | C |
| ATOM | 549 | CD | GLU | | 84 | 27.934 | 83.753 | −70.897 | 1.00 | 31.29 | A | C |
| ATOM | 550 | OE1 | GLU | | 84 | 27.057 | 84.631 | −71.125 | 1.00 | 31.31 | A | O |
| ATOM | 551 | OE2 | GLU | | 84 | 27.710 | 82.516 | −70.902 | 1.00 | 32.34 | A | O |
| ATOM | 552 | C | GLU | | 84 | 31.677 | 86.764 | −72.378 | 1.00 | 22.85 | A | C |
| ATOM | 553 | O | GLU | | 84 | 32.456 | 86.308 | −73.234 | 1.00 | 21.98 | A | O |
| ATOM | 554 | N | ALA | | 85 | 31.147 | 87.973 | −72.460 | 1.00 | 20.86 | A | N |
| ATOM | 555 | CA | ALA | | 85 | 31.560 | 88.899 | −73.513 | 1.00 | 21.57 | A | C |
| ATOM | 556 | CB | ALA | | 85 | 30.785 | 90.189 | −73.405 | 1.00 | 21.75 | A | C |
| ATOM | 557 | C | ALA | | 85 | 33.065 | 89.193 | −73.342 | 1.00 | 21.21 | A | C |
| ATOM | 558 | O | ALA | | 85 | 33.830 | 89.256 | −74.330 | 1.00 | 18.50 | A | O |
| ATOM | 559 | N | LEU | | 86 | 33.484 | 89.420 | −72.099 | 1.00 | 19.89 | A | N |
| ATOM | 560 | CA | LEU | | 86 | 34.902 | 89.710 | −71.884 | 1.00 | 19.31 | A | C |
| ATOM | 561 | CB | LEU | | 86 | 35.158 | 90.096 | −70.438 | 1.00 | 18.18 | A | C |
| ATOM | 562 | CG | LEU | | 86 | 36.644 | 90.291 | −70.024 | 1.00 | 21.44 | A | C |
| ATOM | 563 | CD1 | LEU | | 86 | 37.321 | 91.300 | −70.950 | 1.00 | 21.16 | A | C |
| ATOM | 564 | CD2 | LEU | | 86 | 36.684 | 90.836 | −68.580 | 1.00 | 22.24 | A | C |
| ATOM | 565 | C | LEU | | 86 | 35.740 | 88.480 | −72.287 | 1.00 | 20.01 | A | C |
| ATOM | 566 | O | LEU | | 86 | 36.824 | 88.617 | −72.881 | 1.00 | 18.95 | A | O |
| ATOM | 567 | N | GLU | | 87 | 35.249 | 87.280 | −71.983 | 1.00 | 21.21 | A | N |
| ATOM | 568 | CA | GLU | | 87 | 36.035 | 86.102 | −72.348 | 1.00 | 23.45 | A | C |
| ATOM | 569 | CB | GLU | | 87 | 35.455 | 84.796 | −71.778 | 1.00 | 26.82 | A | C |
| ATOM | 570 | CG | GLU | | 87 | 35.403 | 84.730 | −70.237 | 1.00 | 35.81 | A | C |
| ATOM | 571 | CD | GLU | | 87 | 36.113 | 85.892 | −69.493 | 1.00 | 41.42 | A | C |
| ATOM | 572 | OE1 | GLU | | 87 | 37.352 | 86.104 | −69.706 | 1.00 | 46.73 | A | O |
| ATOM | 573 | OE2 | GLU | | 87 | 35.432 | 86.582 | −68.662 | 1.00 | 43.01 | A | O |
| ATOM | 574 | C | GLU | | 87 | 36.186 | 85.991 | −73.863 | 1.00 | 23.05 | A | C |
| ATOM | 575 | O | GLU | | 87 | 37.267 | 85.593 | −74.352 | 1.00 | 20.62 | A | O |
| ATOM | 576 | N | LEU | | 88 | 35.126 | 86.332 | −74.605 | 1.00 | 19.34 | A | N |
| ATOM | 577 | CA | LEU | | 88 | 35.194 | 86.292 | −76.062 | 1.00 | 20.09 | A | C |
| ATOM | 578 | CB | LEU | | 88 | 33.831 | 86.629 | −76.691 | 1.00 | 20.82 | A | C |
| ATOM | 579 | CG | LEU | | 88 | 32.813 | 85.475 | −76.628 | 1.00 | 22.90 | A | C |
| ATOM | 580 | CD1 | LEU | | 88 | 31.385 | 86.026 | −76.718 | 1.00 | 22.73 | A | C |
| ATOM | 581 | CD2 | LEU | | 88 | 33.105 | 84.479 | −77.755 | 1.00 | 24.12 | A | C |
| ATOM | 582 | C | LEU | | 88 | 36.221 | 87.306 | −76.569 | 1.00 | 18.41 | A | C |
| ATOM | 583 | O | LEU | | 88 | 37.032 | 86.993 | −77.461 | 1.00 | 17.91 | A | O |
| ATOM | 584 | N | ILE | | 89 | 36.152 | 88.520 | −76.032 | 1.00 | 18.04 | A | N |
| ATOM | 585 | CA | ILE | | 89 | 37.079 | 89.597 | −76.411 | 1.00 | 18.31 | A | C |
| ATOM | 586 | CB | ILE | | 89 | 36.781 | 90.867 | −75.587 | 1.00 | 17.73 | A | C |
| ATOM | 587 | CG2 | ILE | | 89 | 37.993 | 91.797 | −75.574 | 1.00 | 16.60 | A | C |
| ATOM | 588 | CG1 | ILE | | 89 | 35.557 | 91.613 | −76.176 | 1.00 | 16.55 | A | C |
| ATOM | 589 | CD1 | ILE | | 89 | 35.886 | 92.467 | −77.435 | 1.00 | 16.65 | A | C |
| ATOM | 590 | C | ILE | | 89 | 38.534 | 89.120 | −76.164 | 1.00 | 19.24 | A | C |
| ATOM | 591 | O | ILE | | 89 | 39.429 | 89.316 | −77.003 | 1.00 | 16.39 | A | O |
| ATOM | 592 | N | ASN | | 90 | 38.767 | 88.547 | −74.989 | 1.00 | 18.26 | A | N |
| ATOM | 593 | CA | ASN | | 90 | 40.106 | 88.036 | −74.659 | 1.00 | 19.40 | A | C |
| ATOM | 594 | CB | ASN | | 90 | 40.133 | 87.413 | −73.260 | 1.00 | 20.55 | A | C |
| ATOM | 595 | CG | ASN | | 90 | 40.050 | 88.444 | −72.139 | 1.00 | 23.18 | A | C |
| ATOM | 596 | OD1 | ASN | | 90 | 40.467 | 89.606 | −72.270 | 1.00 | 24.24 | A | O |
| ATOM | 597 | ND2 | ASN | | 90 | 39.530 | 88.004 | −71.010 | 1.00 | 27.66 | A | N |
| ATOM | 598 | C | ASN | | 90 | 40.566 | 86.989 | −75.672 | 1.00 | 18.92 | A | C |
| ATOM | 599 | O | ASN | | 90 | 41.720 | 87.001 | −76.046 | 1.00 | 18.63 | A | O |
| ATOM | 600 | N | GLY | | 91 | 39.672 | 86.090 | −76.098 | 1.00 | 17.00 | A | N |
| ATOM | 601 | CA | GLY | | 91 | 40.017 | 85.068 | −77.079 | 1.00 | 17.60 | A | C |
| ATOM | 602 | C | GLY | | 91 | 40.368 | 85.689 | −78.431 | 1.00 | 18.65 | A | C |
| ATOM | 603 | O | GLY | | 91 | 41.306 | 85.249 | −79.134 | 1.00 | 17.01 | A | O |
| ATOM | 604 | N | TYR | | 92 | 39.617 | 86.717 | −78.837 | 1.00 | 16.91 | A | N |
| ATOM | 605 | CA | TYR | | 92 | 39.907 | 87.360 | −80.110 | 1.00 | 15.12 | A | C |
| ATOM | 606 | CB | TYR | | 92 | 38.832 | 88.425 | −80.439 | 1.00 | 16.57 | A | C |
| ATOM | 607 | CG | TYR | | 92 | 37.459 | 87.856 | −80.687 | 1.00 | 18.85 | A | C |
| ATOM | 608 | CD1 | TYR | | 92 | 36.308 | 88.546 | −80.289 | 1.00 | 20.18 | A | C |
| ATOM | 609 | CE1 | TYR | | 92 | 35.037 | 88.004 | −80.488 | 1.00 | 21.76 | A | C |
| ATOM | 610 | CD2 | TYR | | 92 | 37.302 | 86.623 | −81.298 | 1.00 | 20.52 | A | C |
| ATOM | 611 | CE2 | TYR | | 92 | 36.056 | 86.086 | −81.500 | 1.00 | 21.54 | A | C |
| ATOM | 612 | CZ | TYR | | 92 | 34.929 | 86.778 | −81.092 | 1.00 | 22.64 | A | C |
| ATOM | 613 | OH | TYR | | 92 | 33.701 | 86.205 | −81.281 | 1.00 | 21.98 | A | O |
| ATOM | 614 | C | TYR | | 92 | 41.277 | 88.047 | −80.030 | 1.00 | 14.32 | A | C |
| ATOM | 615 | O | TYR | | 92 | 42.028 | 88.036 | −80.989 | 1.00 | 14.86 | A | O |
| ATOM | 616 | N | ILE | | 93 | 41.581 | 88.661 | −78.893 | 1.00 | 13.57 | A | N |
| ATOM | 617 | CA | ILE | | 93 | 42.867 | 89.344 | −78.743 | 1.00 | 13.84 | A | C |
| ATOM | 618 | CB | ILE | | 93 | 42.946 | 90.086 | −77.374 | 1.00 | 12.27 | A | C |
| ATOM | 619 | CG2 | ILE | | 93 | 44.425 | 90.497 | −77.043 | 1.00 | 13.10 | A | C |
| ATOM | 620 | CG1 | ILE | | 93 | 42.101 | 91.371 | −77.438 | 1.00 | 11.51 | A | C |
| ATOM | 621 | CD1 | ILE | | 93 | 41.975 | 92.113 | −76.070 | 1.00 | 15.66 | A | C |
| ATOM | 622 | C | ILE | | 93 | 43.994 | 88.313 | −78.867 | 1.00 | 16.30 | A | C |
| ATOM | 623 | O | ILE | | 93 | 45.030 | 88.572 | −79.513 | 1.00 | 13.42 | A | O |
| ATOM | 624 | N | GLN | | 94 | 43.771 | 87.135 | −78.268 | 1.00 | 15.22 | A | N |
| ATOM | 625 | CA | GLN | | 94 | 44.777 | 86.082 | −78.323 | 1.00 | 18.11 | A | C |
| ATOM | 626 | CB | GLN | | 94 | 44.337 | 84.847 | −77.506 | 1.00 | 18.32 | A | C |
| ATOM | 627 | CG | GLN | | 94 | 44.582 | 85.052 | −76.034 | 1.00 | 23.10 | A | C |
| ATOM | 628 | CD | GLN | | 94 | 44.162 | 83.845 | −75.186 | 1.00 | 31.08 | A | C |
| ATOM | 629 | OE1 | GLN | | 94 | 44.740 | 83.594 | −74.134 | 1.00 | 35.10 | A | O |
| ATOM | 630 | NE2 | GLN | | 94 | 43.160 | 83.102 | −75.645 | 1.00 | 30.41 | A | N |
| ATOM | 631 | C | GLN | | 94 | 45.053 | 85.680 | −79.732 | 1.00 | 17.69 | A | C |
| ATOM | 632 | O | GLN | | 94 | 46.201 | 85.571 | −80.127 | 1.00 | 18.29 | A | O |
| ATOM | 633 | N | LYS | | 95 | 43.996 | 85.452 | −80.500 | 1.00 | 17.93 | A | N |
| ATOM | 634 | CA | LYS | | 95 | 44.168 | 85.052 | −81.863 | 1.00 | 18.01 | A | C |
| ATOM | 635 | CB | LYS | | 95 | 42.824 | 84.583 | −82.439 | 1.00 | 22.92 | A | C |
| ATOM | 636 | CG | LYS | | 95 | 42.403 | 83.262 | −81.850 | 1.00 | 29.18 | A | C |
| ATOM | 637 | CD | LYS | | 95 | 41.297 | 82.644 | −82.658 | 1.00 | 35.44 | A | C |
| ATOM | 638 | CE | LYS | | 95 | 41.106 | 81.177 | −82.253 | 1.00 | 39.10 | A | C |
| ATOM | 639 | NZ | LYS | | 95 | 40.194 | 80.481 | −83.215 | 1.00 | 44.13 | A | N |
| ATOM | 640 | C | LYS | | 95 | 44.753 | 86.125 | −82.744 | 1.00 | 15.91 | A | C |
| ATOM | 641 | O | LYS | | 95 | 45.429 | 85.821 | −83.728 | 1.00 | 18.02 | A | O |
| ATOM | 642 | N | ILE | | 96 | 44.458 | 87.393 | −82.446 | 1.00 | 14.05 | A | N |
| ATOM | 643 | CA | ILE | | 96 | 45.013 | 88.490 | −83.244 | 1.00 | 14.33 | A | C |
| ATOM | 644 | CB | ILE | | 96 | 44.286 | 89.846 | −82.904 | 1.00 | 14.29 | A | C |
| ATOM | 645 | CG2 | ILE | | 96 | 45.075 | 91.034 | −83.464 | 1.00 | 15.83 | A | C |
| ATOM | 646 | CG1 | ILE | | 96 | 42.825 | 89.797 | −83.454 | 1.00 | 14.59 | A | C |
| ATOM | 647 | CD1 | ILE | | 96 | 41.890 | 90.934 | −82.949 | 1.00 | 16.80 | A | C |
| ATOM | 648 | C | ILE | | 96 | 46.511 | 88.602 | −82.931 | 1.00 | 14.77 | A | C |
| ATOM | 649 | O | ILE | | 96 | 47.343 | 88.742 | −83.828 | 1.00 | 16.25 | A | O |
| ATOM | 650 | N | LYS | | 97 | 46.862 | 88.538 | −81.661 | 1.00 | 14.85 | A | N |
| ATOM | 651 | CA | LYS | | 97 | 48.283 | 88.687 | −81.307 | 1.00 | 16.64 | A | C |
| ATOM | 652 | CB | LYS | | 97 | 48.450 | 88.841 | −79.781 | 1.00 | 15.69 | A | C |
| ATOM | 653 | CG | LYS | | 97 | 48.026 | 90.219 | −79.232 | 1.00 | 18.98 | A | C |
| ATOM | 654 | CD | LYS | | 97 | 48.628 | 90.407 | −77.809 | 1.00 | 17.75 | A | C |
| ATOM | 655 | CE | LYS | | 97 | 48.518 | 91.845 | −77.332 | 1.00 | 20.16 | A | C |
| ATOM | 656 | NZ | LYS | | 97 | 48.987 | 91.992 | −75.918 | 1.00 | 19.92 | A | N |
| ATOM | 657 | C | LYS | | 97 | 49.159 | 87.526 | −81.781 | 1.00 | 16.39 | A | C |
| ATOM | 658 | O | LYS | | 97 | 50.335 | 87.723 | −82.076 | 1.00 | 19.17 | A | O |
| ATOM | 659 | N | SER | | 98 | 48.575 | 86.344 | −81.833 | 1.00 | 18.08 | A | N |
| ATOM | 660 | CA | SER | | 98 | 49.287 | 85.154 | −82.287 | 1.00 | 18.44 | A | C |
| ATOM | 661 | CB | SER | | 98 | 48.559 | 83.907 | −81.789 | 1.00 | 18.84 | A | C |
| ATOM | 662 | OG | SER | | 98 | 47.343 | 83.703 | −82.490 | 1.00 | 18.67 | A | O |
| ATOM | 663 | C | SER | | 98 | 49.398 | 85.126 | −83.806 | 1.00 | 20.20 | A | C |
| ATOM | 664 | O | SER | | 98 | 50.208 | 84.383 | −84.377 | 1.00 | 19.36 | A | O |
| ATOM | 665 | N | GLY | | 99 | 48.613 | 85.955 | −84.487 | 1.00 | 19.12 | A | N |
| ATOM | 666 | CA | GLY | | 99 | 48.654 | 85.930 | −85.934 | 1.00 | 21.30 | A | C |
| ATOM | 667 | C | GLY | | 99 | 47.751 | 84.838 | −86.510 | 1.00 | 22.45 | A | C |
| ATOM | 668 | O | GLY | | 99 | 47.693 | 84.679 | −87.719 | 1.00 | 23.65 | A | O |
| ATOM | 669 | N | GLU | | 100 | 47.028 | 84.120 | −85.656 | 1.00 | 22.88 | A | N |
| ATOM | 670 | CA | GLU | | 100 | 46.131 | 83.048 | −86.083 | 1.00 | 25.56 | A | C |
| ATOM | 671 | CB | GLU | | 100 | 45.623 | 82.261 | −84.859 | 1.00 | 27.67 | A | C |
| ATOM | 672 | CG | GLU | | 100 | 46.744 | 81.404 | −84.257 | 1.00 | 36.32 | A | C |
| ATOM | 673 | CD | GLU | | 100 | 46.420 | 80.731 | −82.906 | 1.00 | 39.72 | A | C |
| ATOM | 674 | OE1 | GLU | | 100 | 47.106 | 79.729 | −82.590 | 1.00 | 45.08 | A | O |
| ATOM | 675 | OE2 | GLU | | 100 | 45.536 | 81.183 | −82.146 | 1.00 | 41.22 | A | O |
| ATOM | 676 | C | GLU | | 100 | 44.963 | 83.591 | −86.894 | 1.00 | 26.28 | A | C |
| ATOM | 677 | O | GLU | | 100 | 44.494 | 82.932 | −87.812 | 1.00 | 24.89 | A | O |
| ATOM | 678 | N | GLU | | 101 | 44.540 | 84.821 | −86.584 | 1.00 | 25.76 | A | N |
| ATOM | 679 | CA | GLU | | 101 | 43.444 | 85.505 | −87.266 | 1.00 | 27.56 | A | C |
| ATOM | 680 | CB | GLU | | 101 | 42.169 | 85.341 | −86.433 | 1.00 | 32.23 | A | C |
| ATOM | 681 | CG | GLU | | 101 | 41.602 | 83.977 | −86.497 | 1.00 | 37.74 | A | C |
| ATOM | 682 | CD | GLU | | 101 | 40.675 | 83.881 | −87.668 | 1.00 | 40.61 | A | C |
| ATOM | 683 | OE1 | GLU | | 101 | 41.053 | 84.380 | −88.751 | 1.00 | 43.86 | A | O |
| ATOM | 684 | OE2 | GLU | | 101 | 39.575 | 83.325 | −87.501 | 1.00 | 42.27 | A | O |
| ATOM | 685 | C | GLU | | 101 | 43.722 | 87.010 | −87.338 | 1.00 | 26.45 | A | C |
| ATOM | 686 | O | GLU | | 101 | 44.282 | 87.552 | −86.392 | 1.00 | 28.63 | A | O |
| ATOM | 687 | N | ASP | | 102 | 43.373 | 87.715 | −88.410 | 1.00 | 23.17 | A | N |
| ATOM | 688 | CA | ASP | | 102 | 43.634 | 89.132 | −88.279 | 1.00 | 23.62 | A | C |
| ATOM | 689 | CB | ASP | | 102 | 44.232 | 89.796 | −89.519 | 1.00 | 29.72 | A | C |
| ATOM | 690 | CG | ASP | | 102 | 43.482 | 89.523 | −90.763 | 1.00 | 31.60 | A | C |
| ATOM | 691 | OD1 | ASP | | 102 | 42.233 | 89.531 | −90.699 | 1.00 | 34.60 | A | O |
| ATOM | 692 | OD2 | ASP | | 102 | 44.164 | 89.321 | −91.820 | 1.00 | 33.36 | A | O |
| ATOM | 693 | C | ASP | | 102 | 42.402 | 89.868 | −87.789 | 1.00 | 21.92 | A | C |
| ATOM | 694 | O | ASP | | 102 | 41.286 | 89.352 | −87.837 | 1.00 | 19.65 | A | O |
| ATOM | 695 | N | PHE | | 103 | 42.649 | 91.045 | −87.258 | 1.00 | 19.45 | A | N |
| ATOM | 696 | CA | PHE | | 103 | 41.622 | 91.899 | −86.696 | 1.00 | 20.74 | A | C |
| ATOM | 697 | CB | PHE | | 103 | 42.217 | 93.268 | −86.409 | 1.00 | 22.01 | A | C |
| ATOM | 698 | CG | PHE | | 103 | 41.280 | 94.204 | −85.727 | 1.00 | 24.40 | A | C |
| ATOM | 699 | CD1 | PHE | | 103 | 41.300 | 94.317 | −84.344 | 1.00 | 24.41 | A | C |
| ATOM | 700 | CD2 | PHE | | 103 | 40.316 | 94.908 | −86.458 | 1.00 | 27.30 | A | C |
| ATOM | 701 | CE1 | PHE | | 103 | 40.365 | 95.109 | −83.699 | 1.00 | 28.02 | A | C |
| ATOM | 702 | CE2 | PHE | | 103 | 39.365 | 95.714 | −85.809 | 1.00 | 25.93 | A | C |
| ATOM | 703 | CZ | PHE | | 103 | 39.397 | 95.804 | −84.438 | 1.00 | 27.24 | A | C |
| ATOM | 704 | C | PHE | | 103 | 40.432 | 92.065 | −87.634 | 1.00 | 19.25 | A | C |
| ATOM | 705 | O | PHE | | 103 | 39.286 | 91.884 | −87.235 | 1.00 | 17.96 | A | O |
| ATOM | 706 | N | GLU | | 104 | 40.730 | 92.425 | −88.875 | 1.00 | 19.54 | A | N |
| ATOM | 707 | CA | GLU | | 104 | 39.714 | 92.678 | −89.881 | 1.00 | 19.94 | A | C |
| ATOM | 708 | CB | GLU | | 104 | 40.358 | 93.015 | −91.252 | 1.00 | 20.36 | A | C |
| ATOM | 709 | CG | GLU | | 104 | 41.122 | 94.309 | −91.247 | 1.00 | 21.60 | A | C |
| ATOM | 710 | CD | GLU | | 104 | 42.580 | 94.139 | −90.836 | 1.00 | 22.92 | A | C |
| ATOM | 711 | OE1 | GLU | | 104 | 42.963 | 93.137 | −90.137 | 1.00 | 19.61 | A | O |
| ATOM | 712 | OE2 | GLU | | 104 | 43.333 | 95.046 | −91.233 | 1.00 | 25.86 | A | O |
| ATOM | 713 | C | GLU | | 104 | 38.768 | 91.533 | −90.089 | 1.00 | 20.29 | A | C |
| ATOM | 714 | O | GLU | | 104 | 37.547 | 91.756 | −90.253 | 1.00 | 19.85 | A | O |
| ATOM | 715 | N | SER | | 105 | 39.315 | 90.320 | −90.097 | 1.00 | 19.38 | A | N |
| ATOM | 716 | CA | SER | | 105 | 38.512 | 89.129 | −90.322 | 1.00 | 21.13 | A | C |
| ATOM | 717 | CB | SER | | 105 | 39.442 | 87.955 | −90.610 | 1.00 | 23.65 | A | C |
| ATOM | 718 | OG | SER | | 105 | 38.757 | 86.739 | −90.518 | 1.00 | 27.30 | A | O |
| ATOM | 719 | C | SER | | 105 | 37.559 | 88.835 | −89.154 | 1.00 | 20.00 | A | C |
| ATOM | 720 | O | SER | | 105 | 36.374 | 88.507 | −89.353 | 1.00 | 19.10 | A | O |
| ATOM | 721 | N | LEU | | 106 | 38.076 | 88.951 | −87.933 | 1.00 | 19.14 | A | N |
| ATOM | 722 | CA | LEU | | 106 | 37.280 | 88.716 | −86.728 | 1.00 | 16.75 | A | C |
| ATOM | 723 | CB | LEU | | 106 | 38.196 | 88.672 | −85.496 | 1.00 | 16.16 | A | C |
| ATOM | 724 | CG | LEU | | 106 | 39.208 | 87.493 | −85.464 | 1.00 | 15.50 | A | C |
| ATOM | 725 | CD1 | LEU | | 106 | 40.122 | 87.595 | −84.175 | 1.00 | 12.05 | A | C |
| ATOM | 726 | CD2 | LEU | | 106 | 38.453 | 86.179 | −85.471 | 1.00 | 14.15 | A | C |
| ATOM | 727 | C | LEU | | 106 | 36.250 | 89.835 | −86.577 | 1.00 | 15.59 | A | C |
| ATOM | 728 | O | LEU | | 106 | 35.121 | 89.576 | −86.117 | 1.00 | 15.74 | A | O |
| ATOM | 729 | N | ALA | | 107 | 36.607 | 91.065 | −86.931 | 1.00 | 15.57 | A | N |
| ATOM | 730 | CA | ALA | | 107 | 35.601 | 92.145 | −86.828 | 1.00 | 16.29 | A | C |
| ATOM | 731 | CB | ALA | | 107 | 36.199 | 93.498 | −87.126 | 1.00 | 16.43 | A | C |
| ATOM | 732 | C | ALA | | 107 | 34.455 | 91.897 | −87.827 | 1.00 | 18.03 | A | C |
| ATOM | 733 | O | ALA | | 107 | 33.270 | 92.080 | −87.470 | 1.00 | 15.46 | A | O |
| ATOM | 734 | N | SER | | 108 | 34.803 | 91.489 | −89.054 | 1.00 | 15.45 | A | N |
| ATOM | 735 | CA | SER | | 108 | 33.783 | 91.247 | −90.078 | 1.00 | 18.30 | A | C |
| ATOM | 736 | CB | SER | | 108 | 34.423 | 90.830 | −91.408 | 1.00 | 16.85 | A | C |
| ATOM | 737 | OG | SER | | 108 | 35.161 | 91.927 | −91.924 | 1.00 | 25.62 | A | O |
| ATOM | 738 | C | SER | | 108 | 32.839 | 90.123 | −89.639 | 1.00 | 19.47 | A | C |
| ATOM | 739 | O | SER | | 108 | 31.632 | 90.162 | −89.923 | 1.00 | 20.31 | A | O |
| ATOM | 740 | N | GLN | | 109 | 33.392 | 89.118 | −88.976 | 1.00 | 18.53 | A | N |
| ATOM | 741 | CA | GLN | | 109 | 32.589 | 87.992 | −88.525 | 1.00 | 21.76 | A | C |
| ATOM | 742 | CB | GLN | | 109 | 33.467 | 86.754 | −88.376 | 1.00 | 22.14 | A | C |
| ATOM | 743 | CG | GLN | | 109 | 33.979 | 86.202 | −89.702 | 1.00 | 26.08 | A | C |
| ATOM | 744 | CD | GLN | | 109 | 35.046 | 85.135 | −89.499 | 1.00 | 28.34 | A | C |
| ATOM | 745 | OE1 | GLN | | 109 | 36.259 | 85.392 | −89.634 | 1.00 | 30.37 | A | O |
| ATOM | 746 | NE2 | GLN | | 109 | 34.609 | 83.948 | −89.154 | 1.00 | 26.64 | A | N |
| ATOM | 747 | C | GLN | | 109 | 31.856 | 88.173 | −87.204 | 1.00 | 21.62 | A | C |
| ATOM | 748 | O | GLN | | 109 | 30.716 | 87.761 | −87.062 | 1.00 | 24.00 | A | O |
| ATOM | 749 | N | PHE | | 110 | 32.517 | 88.804 | −86.245 | 1.00 | 19.03 | A | N |
| ATOM | 750 | CA | PHE | | 110 | 31.974 | 88.843 | −84.912 | 1.00 | 19.99 | A | C |
| ATOM | 751 | CB | PHE | | 110 | 32.915 | 88.057 | −84.013 | 1.00 | 18.33 | A | C |
| ATOM | 752 | CG | PHE | | 110 | 33.222 | 86.698 | −84.524 | 1.00 | 20.90 | A | C |
| ATOM | 753 | CD1 | PHE | | 110 | 34.529 | 86.356 | −84.880 | 1.00 | 20.70 | A | C |
| ATOM | 754 | CD2 | PHE | | 110 | 32.204 | 85.764 | −84.702 | 1.00 | 20.57 | A | C |
| ATOM | 755 | CE1 | PHE | | 110 | 34.823 | 85.090 | −85.416 | 1.00 | 22.92 | A | C |
| ATOM | 756 | CE2 | PHE | | 110 | 32.470 | 84.492 | −85.239 | 1.00 | 22.53 | A | C |
| ATOM | 757 | CZ | PHE | | 110 | 33.783 | 84.147 | −85.601 | 1.00 | 24.64 | A | C |
| ATOM | 758 | C | PHE | | 110 | 31.628 | 90.114 | −84.205 | 1.00 | 19.17 | A | C |
| ATOM | 759 | O | PHE | | 110 | 31.103 | 90.070 | −83.104 | 1.00 | 20.19 | A | O |
| ATOM | 760 | N | SER | | 111 | 31.911 | 91.243 | −84.805 | 1.00 | 17.72 | A | N |
| ATOM | 761 | CA | SER | | 111 | 31.630 | 92.484 | −84.133 | 1.00 | 16.69 | A | C |
| ATOM | 762 | CB | SER | | 111 | 32.268 | 93.619 | −84.866 | 1.00 | 15.75 | A | C |
| ATOM | 763 | OG | SER | | 111 | 32.005 | 94.836 | −84.172 | 1.00 | 16.48 | A | O |
| ATOM | 764 | C | SER | | 111 | 30.110 | 92.728 | −84.120 | 1.00 | 19.02 | A | C |
| ATOM | 765 | O | SER | | 111 | 29.458 | 92.581 | −85.148 | 1.00 | 17.71 | A | O |
| ATOM | 766 | N | ASP | | 112 | 29.597 | 93.155 | −82.974 | 1.00 | 19.64 | A | N |
| ATOM | 767 | CA | ASP | | 112 | 28.162 | 93.421 | −82.828 | 1.00 | 21.94 | A | C |
| ATOM | 768 | CB | ASP | | 112 | 27.710 | 93.187 | −81.382 | 1.00 | 20.26 | A | C |
| ATOM | 769 | CG | ASP | | 112 | 27.510 | 91.727 | −81.076 | 1.00 | 22.78 | A | C |
| ATOM | 770 | OD1 | ASP | | 112 | 26.877 | 91.038 | −81.900 | 1.00 | 21.51 | A | O |
| ATOM | 771 | OD2 | ASP | | 112 | 27.946 | 91.265 | −79.999 | 1.00 | 20.58 | A | O |
| ATOM | 772 | C | ASP | | 112 | 27.819 | 94.851 | −83.228 | 1.00 | 23.24 | A | C |
| ATOM | 773 | O | ASP | | 112 | 26.864 | 95.425 | −82.710 | 1.00 | 25.37 | A | O |
| ATOM | 774 | N | CYS | | 113 | 28.600 | 95.442 | −84.116 | 1.00 | 23.42 | A | N |
| ATOM | 775 | CA | CYS | | 113 | 28.295 | 96.775 | −84.584 | 1.00 | 23.67 | A | C |
| ATOM | 776 | CB | CYS | | 113 | 29.417 | 97.733 | −84.188 | 1.00 | 21.58 | A | C |
| ATOM | 777 | SG | CYS | | 113 | 29.228 | 99.432 | −84.677 | 1.00 | 25.70 | A | S |
| ATOM | 778 | C | CYS | | 113 | 28.180 | 96.638 | −86.102 | 1.00 | 23.54 | A | C |
| ATOM | 779 | O | CYS | | 113 | 28.789 | 95.744 | −86.698 | 1.00 | 24.58 | A | O |
| ATOM | 780 | N | SER | | 114 | 27.401 | 97.510 | −86.726 | 1.00 | 23.60 | A | N |
| ATOM | 781 | CA | SER | | 114 | 27.226 | 97.459 | −88.162 | 1.00 | 24.32 | A | C |
| ATOM | 782 | CB | SER | | 114 | 26.129 | 98.450 | −88.603 | 1.00 | 27.32 | A | C |
| ATOM | 783 | OG | SER | | 114 | 26.330 | 99.745 | −88.030 | 1.00 | 32.64 | A | O |
| ATOM | 784 | C | SER | | 114 | 28.541 | 97.740 | −88.894 | 1.00 | 23.82 | A | C |
| ATOM | 785 | O | SER | | 114 | 28.681 | 97.377 | −90.069 | 1.00 | 21.84 | A | O |
| ATOM | 786 | N | SER | | 115 | 29.479 | 98.412 | −88.212 | 1.00 | 20.29 | A | N |
| ATOM | 787 | CA | SER | | 115 | 30.794 | 98.686 | −88.794 | 1.00 | 19.79 | A | C |
| ATOM | 788 | CB | SER | | 115 | 31.661 | 99.455 | −87.798 | 1.00 | 18.86 | A | C |
| ATOM | 789 | OG | SER | | 115 | 31.608 | 98.799 | −86.558 | 1.00 | 17.56 | A | O |
| ATOM | 790 | C | SER | | 115 | 31.508 | 97.391 | −89.191 | 1.00 | 18.21 | A | C |
| ATOM | 791 | O | SER | | 115 | 32.498 | 97.439 | −89.920 | 1.00 | 17.69 | A | O |
| ATOM | 792 | N | ALA | | 116 | 31.008 | 96.247 | −88.730 | 1.00 | 16.84 | A | N |
| ATOM | 793 | CA | ALA | | 116 | 31.568 | 94.955 | −89.098 | 1.00 | 16.89 | A | C |
| ATOM | 794 | CB | ALA | | 116 | 30.697 | 93.794 | −88.535 | 1.00 | 18.80 | A | C |
| ATOM | 795 | C | ALA | | 116 | 31.632 | 94.833 | −90.621 | 1.00 | 18.74 | A | C |
| ATOM | 796 | O | ALA | | 116 | 32.611 | 94.278 | −91.163 | 1.00 | 15.51 | A | O |
| ATOM | 797 | N | LYS | | 117 | 30.617 | 95.369 | −91.316 | 1.00 | 16.34 | A | N |
| ATOM | 798 | CA | LYS | | 117 | 30.579 | 95.234 | −92.777 | 1.00 | 18.77 | A | C |
| ATOM | 799 | CB | LYS | | 117 | 29.214 | 95.690 | −93.344 | 1.00 | 21.57 | A | C |
| ATOM | 800 | CG | LYS | | 117 | 28.049 | 95.082 | −92.672 | 1.00 | 26.80 | A | C |
| ATOM | 801 | CD | LYS | | 117 | 28.121 | 93.599 | −92.651 | 1.00 | 30.11 | A | C |
| ATOM | 802 | CE | LYS | | 117 | 27.146 | 93.072 | −91.565 | 1.00 | 30.28 | A | C |
| ATOM | 803 | NZ | LYS | | 117 | 27.253 | 91.647 | −91.321 | 1.00 | 31.77 | A | N |
| ATOM | 804 | C | LYS | | 117 | 31.694 | 95.990 | −93.479 | 1.00 | 17.87 | A | C |
| ATOM | 805 | O | LYS | | 117 | 31.909 | 95.777 | −94.670 | 1.00 | 18.25 | A | O |
| ATOM | 806 | N | ALA | | 118 | 32.368 | 96.885 | −92.744 | 1.00 | 16.99 | A | N |
| ATOM | 807 | CA | ALA | | 118 | 33.514 | 97.610 | −93.252 | 1.00 | 19.26 | A | C |
| ATOM | 808 | CB | ALA | | 118 | 33.339 | 99.151 | −93.044 | 1.00 | 19.78 | A | C |
| ATOM | 809 | C | ALA | | 118 | 34.797 | 97.132 | −92.545 | 1.00 | 18.77 | A | C |
| ATOM | 810 | O | ALA | | 118 | 35.711 | 97.927 | −92.312 | 1.00 | 17.15 | A | O |
| ATOM | 811 | N | ARG | | 119 | 34.835 | 95.851 | −92.167 | 1.00 | 18.30 | A | N |
| ATOM | 812 | CA | ARG | | 119 | 36.008 | 95.268 | −91.489 | 1.00 | 18.69 | A | C |
| ATOM | 813 | CB | ARG | | 119 | 37.255 | 95.320 | −92.430 | 1.00 | 20.61 | A | C |
| ATOM | 814 | CG | ARG | | 119 | 37.140 | 94.365 | −93.623 | 1.00 | 22.17 | A | C |
| ATOM | 815 | CD | ARG | | 119 | 38.232 | 94.524 | −94.739 | 1.00 | 25.75 | A | C |
| ATOM | 816 | NE | ARG | | 119 | 38.514 | 95.932 | −94.967 | 1.00 | 29.83 | A | N |
| ATOM | 817 | CZ | ARG | | 119 | 37.707 | 96.866 | −95.509 | 1.00 | 32.46 | A | C |
| ATOM | 818 | NH1 | ARG | | 119 | 36.458 | 96.628 | −95.967 | 1.00 | 27.79 | A | N |
| ATOM | 819 | NH2 | ARG | | 119 | 38.156 | 98.116 | −95.532 | 1.00 | 34.51 | A | N |
| ATOM | 820 | C | ARG | | 119 | 36.253 | 96.026 | −90.177 | 1.00 | 19.31 | A | C |
| ATOM | 821 | O | ARG | | 119 | 37.391 | 96.132 | −89.684 | 1.00 | 18.00 | A | O |
| ATOM | 822 | N | GLY | | 120 | 35.161 | 96.576 | −89.630 | 1.00 | 16.58 | A | N |
| ATOM | 823 | CA | GLY | | 120 | 35.217 | 97.301 | −88.381 | 1.00 | 18.25 | A | C |
| ATOM | 824 | C | GLY | | 120 | 35.681 | 98.742 | −88.422 | 1.00 | 16.96 | A | C |
| ATOM | 825 | O | GLY | | 120 | 35.790 | 99.386 | −87.385 | 1.00 | 18.18 | A | O |
| ATOM | 826 | N | ASP | | 121 | 35.912 | 99.269 | −89.614 | 1.00 | 17.17 | A | N |
| ATOM | 827 | CA | ASP | | 121 | 36.418 | 100.634 | −89.759 | 1.00 | 18.90 | A | C |
| ATOM | 828 | CB | ASP | | 121 | 36.880 | 100.867 | −91.216 | 1.00 | 19.58 | A | C |
| ATOM | 829 | CG | ASP | | 121 | 37.288 | 102.320 | −91.468 | 1.00 | 20.04 | A | C |
| ATOM | 830 | OD1 | ASP | | 121 | 38.016 | 102.878 | −90.629 | 1.00 | 19.84 | A | O |
| ATOM | 831 | OD2 | ASP | | 121 | 36.869 | 102.906 | −92.481 | 1.00 | 19.19 | A | O |
| ATOM | 832 | C | ASP | | 121 | 35.365 | 101.678 | −89.377 | 1.00 | 20.15 | A | C |
| ATOM | 833 | O | ASP | | 121 | 34.230 | 101.569 | −89.800 | 1.00 | 20.34 | A | O |
| ATOM | 834 | N | LEU | | 122 | 35.749 | 102.645 | −88.559 | 1.00 | 19.02 | A | N |
| ATOM | 835 | CA | LEU | | 122 | 34.848 | 103.715 | −88.156 | 1.00 | 21.65 | A | C |
| ATOM | 836 | CB | LEU | | 122 | 34.921 | 103.946 | −86.652 | 1.00 | 20.81 | A | C |
| ATOM | 837 | CG | LEU | | 122 | 34.421 | 102.760 | −85.831 | 1.00 | 21.84 | A | C |
| ATOM | 838 | CD1 | LEU | | 122 | 34.583 | 103.084 | −84.365 | 1.00 | 23.75 | A | C |
| ATOM | 839 | CD2 | LEU | | 122 | 32.969 | 102.456 | −86.194 | 1.00 | 22.78 | A | C |
| ATOM | 840 | C | LEU | | 122 | 35.173 | 105.024 | −88.851 | 1.00 | 23.36 | A | C |
| ATOM | 841 | O | LEU | | 122 | 34.465 | 105.998 | −88.677 | 1.00 | 25.30 | A | O |
| ATOM | 842 | N | GLY | | 123 | 36.250 | 105.067 | −89.620 | 1.00 | 25.70 | A | N |
| ATOM | 843 | CA | GLY | | 123 | 36.604 | 106.318 | −90.275 | 1.00 | 25.24 | A | C |
| ATOM | 844 | C | GLY | | 123 | 37.367 | 107.190 | −89.303 | 1.00 | 26.16 | A | C |
| ATOM | 845 | O | GLY | | 123 | 37.671 | 106.748 | −88.174 | 1.00 | 25.30 | A | O |
| ATOM | 846 | N | ALA | | 124 | 37.683 | 108.422 | −89.699 | 1.00 | 24.36 | A | N |
| ATOM | 847 | CA | ALA | | 124 | 38.452 | 109.310 | −88.816 | 1.00 | 26.20 | A | C |
| ATOM | 848 | CB | ALA | | 124 | 39.366 | 110.207 | −89.625 | 1.00 | 26.57 | A | C |
| ATOM | 849 | C | ALA | | 124 | 37.531 | 110.158 | −87.941 | 1.00 | 27.11 | A | C |
| ATOM | 850 | O | ALA | | 124 | 36.406 | 110.460 | −88.339 | 1.00 | 29.63 | A | O |
| ATOM | 851 | N | PHE | | 125 | 37.999 | 110.522 | −86.752 | 1.00 | 27.80 | A | N |
| ATOM | 852 | CA | PHE | | 125 | 37.196 | 111.317 | −85.833 | 1.00 | 29.24 | A | C |
| ATOM | 853 | CB | PHE | | 125 | 36.278 | 110.414 | −84.988 | 1.00 | 29.60 | A | C |
| ATOM | 854 | CG | PHE | | 125 | 37.008 | 109.286 | −84.244 | 1.00 | 31.02 | A | C |
| ATOM | 855 | CD1 | PHE | | 125 | 37.630 | 109.520 | −83.028 | 1.00 | 29.25 | A | C |
| ATOM | 856 | CD2 | PHE | | 125 | 37.039 | 107.993 | −84.770 | 1.00 | 29.85 | A | C |
| ATOM | 857 | CE1 | PHE | | 125 | 38.274 | 108.470 | −82.336 | 1.00 | 30.74 | A | C |
| ATOM | 858 | CE2 | PHE | | 125 | 37.677 | 106.940 | −84.092 | 1.00 | 31.23 | A | C |
| ATOM | 859 | CZ | PHE | | 125 | 38.294 | 107.175 | −82.875 | 1.00 | 28.55 | A | C |
| ATOM | 860 | C | PHE | | 125 | 38.066 | 112.201 | −84.968 | 1.00 | 30.03 | A | C |
| ATOM | 861 | O | PHE | | 125 | 39.290 | 111.985 | −84.820 | 1.00 | 30.86 | A | O |
| ATOM | 862 | N | SER | | 126 | 37.462 | 113.242 | −84.416 | 1.00 | 30.81 | A | N |
| ATOM | 863 | CA | SER | | 126 | 38.220 | 114.160 | −83.575 | 1.00 | 31.58 | A | C |
| ATOM | 864 | CB | SER | | 126 | 37.942 | 115.617 | −84.009 | 1.00 | 33.37 | A | C |
| ATOM | 865 | OG | SER | | 126 | 36.601 | 115.954 | −83.683 | 1.00 | 33.92 | A | O |
| ATOM | 866 | C | SER | | 126 | 37.714 | 113.933 | −82.156 | 1.00 | 31.65 | A | C |
| ATOM | 867 | O | SER | | 126 | 36.757 | 113.178 | −81.938 | 1.00 | 31.14 | A | O |
| ATOM | 868 | N | ARG | | 127 | 38.345 | 114.570 | −81.177 | 1.00 | 33.84 | A | N |
| ATOM | 869 | CA | ARG | | 127 | 37.869 | 114.407 | −79.806 | 1.00 | 34.42 | A | C |
| ATOM | 870 | CB | ARG | | 127 | 38.834 | 115.022 | −78.791 | 1.00 | 34.39 | A | C |
| ATOM | 871 | CG | ARG | | 127 | 39.939 | 114.099 | −78.368 | 1.00 | 33.90 | A | C |
| ATOM | 872 | CD | ARG | | 127 | 40.538 | 114.580 | −77.083 | 1.00 | 33.65 | A | C |
| ATOM | 873 | NE | ARG | | 127 | 41.728 | 113.830 | −76.709 | 1.00 | 33.32 | A | N |
| ATOM | 874 | CZ | ARG | | 127 | 41.734 | 112.760 | −75.920 | 1.00 | 31.01 | A | C |
| ATOM | 875 | NH1 | ARG | | 127 | 40.594 | 112.298 | −75.419 | 1.00 | 29.87 | A | N |
| ATOM | 876 | NH2 | ARG | | 127 | 42.899 | 112.189 | −75.597 | 1.00 | 28.67 | A | N |
| ATOM | 877 | C | ARG | | 127 | 36.555 | 115.136 | −79.730 | 1.00 | 36.57 | A | C |
| ATOM | 878 | O | ARG | | 127 | 36.356 | 116.139 | −80.419 | 1.00 | 36.65 | A | O |
| ATOM | 879 | N | GLY | | 128 | 35.645 | 114.622 | −78.924 | 1.00 | 37.80 | A | N |
| ATOM | 880 | CA | GLY | | 128 | 34.368 | 115.282 | −78.801 | 1.00 | 40.58 | A | C |
| ATOM | 881 | C | GLY | | 128 | 33.303 | 114.678 | −79.688 | 1.00 | 41.54 | A | C |
| ATOM | 882 | O | GLY | | 128 | 32.308 | 115.349 | −79.945 | 1.00 | 42.23 | A | O |
| ATOM | 883 | N | GLN | | 129 | 33.501 | 113.436 | −80.153 | 1.00 | 41.18 | A | N |
| ATOM | 884 | CA | GLN | | 129 | 32.525 | 112.765 | −81.012 | 1.00 | 39.94 | A | C |
| ATOM | 885 | CB | GLN | | 129 | 33.046 | 112.586 | −82.444 | 1.00 | 39.92 | A | C |
| ATOM | 886 | CG | GLN | | 129 | 33.650 | 113.809 | −83.112 | 1.00 | 41.65 | A | C |
| ATOM | 887 | CD | GLN | | 129 | 33.975 | 113.553 | −84.589 | 1.00 | 42.62 | A | C |
| ATOM | 888 | OE1 | GLN | | 129 | 34.806 | 114.236 | −85.192 | 1.00 | 43.40 | A | O |
| ATOM | 889 | NE2 | GLN | | 129 | 33.306 | 112.570 | −85.173 | 1.00 | 43.98 | A | N |
| ATOM | 890 | C | GLN | | 129 | 32.088 | 111.390 | −80.514 | 1.00 | 40.33 | A | C |
| ATOM | 891 | O | GLN | | 129 | 30.917 | 111.067 | −80.629 | 1.00 | 40.78 | A | O |
| ATOM | 892 | N | MET | | 130 | 33.009 | 110.574 | −79.976 | 1.00 | 39.29 | A | N |
| ATOM | 893 | CA | MET | | 130 | 32.678 | 109.206 | −79.509 | 1.00 | 37.91 | A | C |
| ATOM | 894 | CB | MET | | 130 | 33.834 | 108.248 | −79.869 | 1.00 | 38.70 | A | C |
| ATOM | 895 | CG | MET | | 130 | 34.429 | 108.416 | −81.257 | 1.00 | 38.21 | A | C |
| ATOM | 896 | SD | MET | | 130 | 33.246 | 107.975 | −82.546 | 1.00 | 40.84 | A | S |
| ATOM | 897 | CE | MET | | 130 | 33.076 | 106.352 | −82.287 | 1.00 | 40.34 | A | C |
| ATOM | 898 | C | MET | | 130 | 32.423 | 109.043 | −77.989 | 1.00 | 37.09 | A | C |
| ATOM | 899 | O | MET | | 130 | 32.668 | 109.959 | −77.225 | 1.00 | 37.50 | A | O |
| ATOM | 900 | N | GLN | | 131 | 31.936 | 107.879 | −77.564 | 1.00 | 36.12 | A | N |
| ATOM | 901 | CA | GLN | | 131 | 31.767 | 107.617 | −76.132 | 1.00 | 37.22 | A | C |
| ATOM | 902 | CB | GLN | | 131 | 31.288 | 106.186 | −75.890 | 1.00 | 39.10 | A | C |
| ATOM | 903 | CG | GLN | | 131 | 29.774 | 106.026 | −76.033 | 1.00 | 42.50 | A | C |
| ATOM | 904 | CD | GLN | | 131 | 29.310 | 104.582 | −75.864 | 1.00 | 44.70 | A | C |
| ATOM | 905 | OE1 | GLN | | 131 | 28.140 | 104.315 | −75.580 | 1.00 | 47.49 | A | O |
| ATOM | 906 | NE2 | GLN | | 131 | 30.217 | 103.649 | −76.058 | 1.00 | 45.09 | A | N |
| ATOM | 907 | C | GLN | | 131 | 33.167 | 107.847 | −75.519 | 1.00 | 36.77 | A | C |
| ATOM | 908 | O | GLN | | 131 | 34.203 | 107.388 | −76.047 | 1.00 | 34.55 | A | O |
| ATOM | 909 | N | LYS | | 132 | 33.196 | 108.594 | −74.423 | 1.00 | 34.30 | A | N |
| ATOM | 910 | CA | LYS | | 132 | 34.451 | 108.976 | −73.779 | 1.00 | 32.67 | A | C |
| ATOM | 911 | CB | LYS | | 132 | 34.124 | 109.644 | −72.433 | 1.00 | 33.55 | A | C |
| ATOM | 912 | CG | LYS | | 132 | 35.288 | 110.388 | −71.820 | 1.00 | 33.86 | A | C |
| ATOM | 913 | CD | LYS | | 132 | 35.846 | 111.451 | −72.744 | 1.00 | 33.85 | A | C |
| ATOM | 914 | CE | LYS | | 132 | 37.141 | 111.988 | −72.157 | 1.00 | 36.22 | A | C |
| ATOM | 915 | NZ | LYS | | 132 | 37.949 | 112.835 | −73.056 | 1.00 | 36.55 | A | N |
| ATOM | 916 | C | LYS | | 132 | 35.552 | 107.895 | −73.610 | 1.00 | 29.94 | A | C |
| ATOM | 917 | O | LYS | | 132 | 36.725 | 108.138 | −73.933 | 1.00 | 30.20 | A | O |
| ATOM | 918 | N | PRO | | 133 | 35.203 | 106.716 | −73.088 | 1.00 | 27.61 | A | N |
| ATOM | 919 | CD | PRO | | 133 | 33.911 | 106.214 | −72.604 | 1.00 | 28.84 | A | C |
| ATOM | 920 | CA | PRO | | 133 | 36.254 | 105.704 | −72.933 | 1.00 | 27.73 | A | C |
| ATOM | 921 | CB | PRO | | 133 | 35.531 | 104.540 | −72.258 | 1.00 | 27.32 | A | C |
| ATOM | 922 | CG | PRO | | 133 | 34.333 | 105.190 | −71.590 | 1.00 | 29.00 | A | C |
| ATOM | 923 | C | PRO | | 133 | 36.848 | 105.269 | −74.283 | 1.00 | 26.85 | A | C |
| ATOM | 924 | O | PRO | | 133 | 38.056 | 104.996 | −74.381 | 1.00 | 26.46 | A | O |
| ATOM | 925 | N | PHE | | 134 | 35.992 | 105.207 | −75.312 | 1.00 | 25.47 | A | N |
| ATOM | 926 | CA | PHE | | 134 | 36.424 | 104.790 | −76.646 | 1.00 | 23.15 | A | C |
| ATOM | 927 | CB | PHE | | 134 | 35.209 | 104.652 | −77.573 | 1.00 | 20.08 | A | C |
| ATOM | 928 | CG | PHE | | 134 | 35.530 | 104.023 | −78.909 | 1.00 | 19.50 | A | C |
| ATOM | 929 | CD1 | PHE | | 134 | 35.399 | 102.656 | −79.096 | 1.00 | 17.39 | A | C |
| ATOM | 930 | CD2 | PHE | | 134 | 36.015 | 104.790 | −79.951 | 1.00 | 19.22 | A | C |
| ATOM | 931 | CE1 | PHE | | 134 | 35.761 | 102.059 | −80.323 | 1.00 | 20.05 | A | C |
| ATOM | 932 | CE2 | PHE | | 134 | 36.382 | 104.189 | −81.169 | 1.00 | 19.39 | A | C |
| ATOM | 933 | CZ | PHE | | 134 | 36.248 | 102.830 | −81.330 | 1.00 | 17.27 | A | C |
| ATOM | 934 | C | PHE | | 134 | 37.341 | 105.882 | −77.145 | 1.00 | 21.95 | A | C |
| ATOM | 935 | O | PHE | | 134 | 38.435 | 105.635 | −77.674 | 1.00 | 19.32 | A | O |
| ATOM | 936 | N | GLU | | 135 | 36.902 | 107.130 | −76.955 | 1.00 | 20.33 | A | N |
| ATOM | 937 | CA | GLU | | 135 | 37.700 | 108.246 | −77.400 | 1.00 | 20.23 | A | C |
| ATOM | 938 | CB | GLU | | 135 | 37.009 | 109.590 | −77.086 | 1.00 | 20.40 | A | C |
| ATOM | 939 | CG | GLU | | 135 | 37.953 | 110.752 | −77.201 | 1.00 | 24.60 | A | C |
| ATOM | 940 | CD | GLU | | 135 | 37.251 | 112.098 | −76.882 | 1.00 | 29.23 | A | C |
| ATOM | 941 | OE1 | GLU | | 135 | 36.086 | 112.269 | −77.330 | 1.00 | 26.62 | A | O |
| ATOM | 942 | OE2 | GLU | | 135 | 37.881 | 112.938 | −76.198 | 1.00 | 30.65 | A | O |
| ATOM | 943 | C | GLU | | 135 | 39.080 | 108.256 | −76.779 | 1.00 | 20.28 | A | C |
| ATOM | 944 | O | GLU | | 135 | 40.110 | 108.312 | −77.490 | 1.00 | 19.35 | A | O |
| ATOM | 945 | N | ASP | | 136 | 39.115 | 108.233 | −75.449 | 1.00 | 18.56 | A | N |
| ATOM | 946 | CA | ASP | | 136 | 40.397 | 108.265 | −74.751 | 1.00 | 19.44 | A | C |
| ATOM | 947 | CB | ASP | | 136 | 40.179 | 108.205 | −73.248 | 1.00 | 20.59 | A | C |
| ATOM | 948 | CG | ASP | | 136 | 39.663 | 109.537 | −72.690 | 1.00 | 23.97 | A | C |
| ATOM | 949 | OD1 | ASP | | 136 | 39.779 | 110.573 | −73.391 | 1.00 | 22.30 | A | O |
| ATOM | 950 | OD2 | ASP | | 136 | 39.174 | 109.529 | −71.548 | 1.00 | 25.27 | A | O |
| ATOM | 951 | C | ASP | | 136 | 41.350 | 107.140 | −75.155 | 1.00 | 16.85 | A | C |
| ATOM | 952 | O | ASP | | 136 | 42.557 | 107.350 | −75.323 | 1.00 | 18.14 | A | O |
| ATOM | 953 | N | ALA | | 137 | 40.805 | 105.953 | −75.287 | 1.00 | 18.11 | A | N |
| ATOM | 954 | CA | ALA | | 137 | 41.623 | 104.779 | −75.674 | 1.00 | 16.81 | A | C |
| ATOM | 955 | CB | ALA | | 137 | 40.733 | 103.530 | −75.632 | 1.00 | 15.80 | A | C |
| ATOM | 956 | C | ALA | | 137 | 42.149 | 105.033 | −77.113 | 1.00 | 16.85 | A | C |
| ATOM | 957 | O | ALA | | 137 | 43.347 | 104.878 | −77.425 | 1.00 | 16.40 | A | O |
| ATOM | 958 | N | SER | | 138 | 41.258 | 105.486 | −77.990 | 1.00 | 17.57 | A | N |
| ATOM | 959 | CA | SER | | 138 | 41.659 | 105.747 | −79.369 | 1.00 | 18.64 | A | C |
| ATOM | 960 | CB | SER | | 138 | 40.454 | 106.184 | −80.232 | 1.00 | 18.01 | A | C |
| ATOM | 961 | OG | SER | | 138 | 39.505 | 105.151 | −80.304 | 1.00 | 18.74 | A | O |
| ATOM | 962 | C | SER | | 138 | 42.770 | 106.745 | −79.506 | 1.00 | 20.78 | A | C |
| ATOM | 963 | O | SER | | 138 | 43.702 | 106.508 | −80.264 | 1.00 | 20.45 | A | O |
| ATOM | 964 | N | PHE | | 139 | 42.718 | 107.848 | −78.756 | 1.00 | 20.81 | A | N |
| ATOM | 965 | CA | PHE | | 139 | 43.741 | 108.870 | −78.867 | 1.00 | 22.12 | A | C |
| ATOM | 966 | CB | PHE | | 139 | 43.213 | 110.254 | −78.378 | 1.00 | 22.25 | A | C |
| ATOM | 967 | CG | PHE | | 139 | 42.444 | 110.995 | −79.447 | 1.00 | 23.41 | A | C |
| ATOM | 968 | CD1 | PHE | | 139 | 41.127 | 110.697 | −79.694 | 1.00 | 23.40 | A | C |
| ATOM | 969 | CD2 | PHE | | 139 | 43.100 | 111.884 | −80.286 | 1.00 | 24.82 | A | C |
| ATOM | 970 | CE1 | PHE | | 139 | 40.453 | 111.272 | −80.796 | 1.00 | 26.94 | A | C |
| ATOM | 971 | CE2 | PHE | | 139 | 42.465 | 112.453 | −81.360 | 1.00 | 26.50 | A | C |
| ATOM | 972 | CZ | PHE | | 139 | 41.129 | 112.149 | −81.626 | 1.00 | 25.32 | A | C |
| ATOM | 973 | C | PHE | | 139 | 45.021 | 108.521 | −78.143 | 1.00 | 22.42 | A | C |
| ATOM | 974 | O | PHE | | 139 | 46.051 | 109.131 | −78.411 | 1.00 | 24.68 | A | O |
| ATOM | 975 | N | ALA | | 140 | 44.951 | 107.538 | −77.248 | 1.00 | 21.64 | A | N |
| ATOM | 976 | CA | ALA | | 140 | 46.121 | 107.104 | −76.485 | 1.00 | 21.40 | A | C |
| ATOM | 977 | CB | ALA | | 140 | 45.680 | 106.569 | −75.112 | 1.00 | 19.38 | A | C |
| ATOM | 978 | C | ALA | | 140 | 46.923 | 106.040 | −77.253 | 1.00 | 21.95 | A | C |
| ATOM | 979 | O | ALA | | 140 | 48.091 | 105.797 | −76.936 | 1.00 | 21.81 | A | O |
| ATOM | 980 | N | LEU | | 141 | 46.307 | 105.427 | −78.270 | 1.00 | 21.31 | A | N |
| ATOM | 981 | CA | LEU | | 141 | 46.998 | 104.437 | −79.105 | 1.00 | 23.06 | A | C |
| ATOM | 982 | CB | LEU | | 141 | 45.983 | 103.609 | −79.923 | 1.00 | 20.42 | A | C |
| ATOM | 983 | CG | LEU | | 141 | 45.091 | 102.621 | −79.157 | 1.00 | 19.85 | A | C |
| ATOM | 984 | CD1 | LEU | | 141 | 43.983 | 102.129 | −80.109 | 1.00 | 16.71 | A | C |
| ATOM | 985 | CD2 | LEU | | 141 | 45.898 | 101.454 | −78.615 | 1.00 | 19.89 | A | C |
| ATOM | 986 | C | LEU | | 141 | 47.878 | 105.160 | −80.101 | 1.00 | 24.61 | A | C |
| ATOM | 987 | O | LEU | | 141 | 47.560 | 106.279 | −80.496 | 1.00 | 24.42 | A | O |
| ATOM | 988 | N | ARG | | 142 | 48.979 | 104.526 | −80.505 | 1.00 | 26.12 | A | N |
| ATOM | 989 | CA | ARG | | 142 | 49.851 | 105.066 | −81.531 | 1.00 | 26.46 | A | C |
| ATOM | 990 | CB | ARG | | 142 | 51.294 | 104.643 | −81.285 | 1.00 | 29.86 | A | C |
| ATOM | 991 | CG | ARG | | 142 | 51.749 | 104.853 | −79.849 | 1.00 | 33.68 | A | C |
| ATOM | 992 | CD | ARG | | 142 | 53.175 | 104.476 | −79.736 | 1.00 | 37.87 | A | C |
| ATOM | 993 | NE | ARG | | 142 | 53.471 | 103.906 | −78.437 | 1.00 | 40.98 | A | N |
| ATOM | 994 | CZ | ARG | | 142 | 53.194 | 102.658 | −78.082 | 1.00 | 43.11 | A | C |
| ATOM | 995 | NH1 | ARG | | 142 | 52.603 | 101.798 | −78.935 | 1.00 | 43.53 | A | N |
| ATOM | 996 | NH2 | ARG | | 142 | 53.499 | 102.273 | −76.852 | 1.00 | 44.47 | A | N |
| ATOM | 997 | C | ARG | | 142 | 49.340 | 104.404 | −82.810 | 1.00 | 26.06 | A | C |
| ATOM | 998 | O | ARG | | 142 | 48.681 | 103.372 | −82.756 | 1.00 | 22.42 | A | O |
| ATOM | 999 | N | THR | | 143 | 49.634 | 104.993 | −83.953 | 1.00 | 24.61 | A | N |
| ATOM | 1000 | CA | THR | | 143 | 49.194 | 104.415 | −85.202 | 1.00 | 26.62 | A | C |
| ATOM | 1001 | CB | THR | | 143 | 49.725 | 105.219 | −86.374 | 1.00 | 28.69 | A | C |
| ATOM | 1002 | OG1 | THR | | 143 | 49.096 | 106.494 | −86.339 | 1.00 | 33.62 | A | O |
| ATOM | 1003 | CG2 | THR | | 143 | 49.385 | 104.551 | −87.702 | 1.00 | 29.97 | A | C |
| ATOM | 1004 | C | THR | | 143 | 49.702 | 102.993 | −85.310 | 1.00 | 26.07 | A | C |
| ATOM | 1005 | O | THR | | 143 | 50.898 | 102.738 | −85.100 | 1.00 | 23.72 | A | O |
| ATOM | 1006 | N | GLY | | 144 | 48.788 | 102.071 | −85.605 | 1.00 | 22.98 | A | N |
| ATOM | 1007 | CA | GLY | | 144 | 49.170 | 100.678 | −85.735 | 1.00 | 22.71 | A | C |
| ATOM | 1008 | C | GLY | | 144 | 48.924 | 99.849 | −84.485 | 1.00 | 20.73 | A | C |
| ATOM | 1009 | O | GLY | | 144 | 48.859 | 98.612 | −84.549 | 1.00 | 20.00 | A | O |
| ATOM | 1010 | N | GLU | | 145 | 48.771 | 100.525 | −83.343 | 1.00 | 19.85 | A | N |
| ATOM | 1011 | CA | GLU | | 145 | 48.562 | 99.829 | −82.079 | 1.00 | 19.58 | A | C |
| ATOM | 1012 | CB | GLU | | 145 | 49.116 | 100.678 | −80.947 | 1.00 | 20.15 | A | C |
| ATOM | 1013 | CG | GLU | | 145 | 49.025 | 100.035 | −79.590 | 1.00 | 26.73 | A | C |
| ATOM | 1014 | CD | GLU | | 145 | 49.534 | 100.956 | −78.485 | 1.00 | 30.45 | A | C |
| ATOM | 1015 | OE1 | GLU | | 145 | 49.784 | 102.182 | −78.774 | 1.00 | 30.77 | A | O |
| ATOM | 1016 | OE2 | GLU | | 145 | 49.668 | 100.441 | −77.345 | 1.00 | 30.02 | A | O |
| ATOM | 1017 | C | GLU | | 145 | 47.114 | 99.426 | −81.812 | 1.00 | 19.24 | A | C |
| ATOM | 1018 | O | GLU | | 145 | 46.163 | 100.068 | −82.295 | 1.00 | 19.29 | A | O |
| ATOM | 1019 | N | MET | | 146 | 46.960 | 98.336 | −81.086 | 1.00 | 15.79 | A | N |
| ATOM | 1020 | CA | MET | | 146 | 45.655 | 97.778 | −80.721 | 1.00 | 15.54 | A | C |
| ATOM | 1021 | CB | MET | | 146 | 45.555 | 96.301 | −81.140 | 1.00 | 15.92 | A | C |
| ATOM | 1022 | CG | MET | | 146 | 44.195 | 95.641 | −80.857 | 1.00 | 17.65 | A | C |
| ATOM | 1023 | SD | MET | | 146 | 44.192 | 93.964 | −81.526 | 1.00 | 18.77 | A | S |
| ATOM | 1024 | CE | MET | | 146 | 45.080 | 93.104 | −80.239 | 1.00 | 19.12 | A | C |
| ATOM | 1025 | C | MET | | 146 | 45.457 | 97.866 | −79.203 | 1.00 | 14.04 | A | C |
| ATOM | 1026 | O | MET | | 146 | 46.390 | 97.687 | −78.402 | 1.00 | 13.72 | A | O |
| ATOM | 1027 | N | SER | | 147 | 44.225 | 98.110 | −78.805 | 1.00 | 11.92 | A | N |
| ATOM | 1028 | CA | SER | | 147 | 43.921 | 98.204 | −77.386 | 1.00 | 12.79 | A | C |
| ATOM | 1029 | CB | SER | | 147 | 42.628 | 99.018 | −77.196 | 1.00 | 12.46 | A | C |
| ATOM | 1030 | OG | SER | | 147 | 41.475 | 98.257 | −77.621 | 1.00 | 13.56 | A | O |
| ATOM | 1031 | C | SER | | 147 | 43.669 | 96.841 | −76.784 | 1.00 | 12.38 | A | C |
| ATOM | 1032 | O | SER | | 147 | 43.648 | 95.839 | −77.480 | 1.00 | 14.87 | A | O |
| ATOM | 1033 | N | GLY | | 148 | 43.495 | 96.846 | −75.473 | 1.00 | 14.75 | A | N |
| ATOM | 1034 | CA | GLY | | 148 | 43.033 | 95.663 | −74.764 | 1.00 | 15.86 | A | C |
| ATOM | 1035 | C | GLY | | 148 | 41.500 | 95.883 | −74.694 | 1.00 | 16.87 | A | C |
| ATOM | 1036 | O | GLY | | 148 | 40.940 | 96.703 | −75.437 | 1.00 | 16.19 | A | O |
| ATOM | 1037 | N | PRO | | 149 | 40.800 | 95.183 | −73.811 | 1.00 | 18.59 | A | N |
| ATOM | 1038 | CD | PRO | | 149 | 41.311 | 94.147 | −72.911 | 1.00 | 17.63 | A | C |
| ATOM | 1039 | CA | PRO | | 149 | 39.333 | 95.339 | −73.696 | 1.00 | 19.64 | A | C |
| ATOM | 1040 | CB | PRO | | 149 | 38.944 | 94.251 | −72.690 | 1.00 | 19.84 | A | C |
| ATOM | 1041 | CG | PRO | | 149 | 40.092 | 93.220 | −72.808 | 1.00 | 20.94 | A | C |
| ATOM | 1042 | C | PRO | | 149 | 39.027 | 96.758 | −73.205 | 1.00 | 18.98 | A | C |
| ATOM | 1043 | O | PRO | | 149 | 39.612 | 97.208 | −72.219 | 1.00 | 18.83 | A | O |
| ATOM | 1044 | N | VAL | | 150 | 38.137 | 97.454 | −73.908 | 1.00 | 17.89 | A | N |
| ATOM | 1045 | CA | VAL | | 150 | 37.753 | 98.824 | −73.549 | 1.00 | 17.17 | A | C |
| ATOM | 1046 | CB | VAL | | 150 | 38.097 | 99.803 | −74.667 | 1.00 | 16.06 | A | C |
| ATOM | 1047 | CG1 | VAL | | 150 | 37.486 | 101.213 | −74.404 | 1.00 | 16.58 | A | C |
| ATOM | 1048 | CG2 | VAL | | 150 | 39.614 | 99.917 | −74.795 | 1.00 | 18.32 | A | C |
| ATOM | 1049 | C | VAL | | 150 | 36.250 | 98.758 | −73.293 | 1.00 | 19.14 | A | C |
| ATOM | 1050 | O | VAL | | 150 | 35.479 | 98.405 | −74.176 | 1.00 | 18.06 | A | O |
| ATOM | 1051 | N | PHE | | 151 | 35.854 | 99.078 | −72.067 | 1.00 | 19.43 | A | N |
| ATOM | 1052 | CA | PHE | | 151 | 34.460 | 98.995 | −71.687 | 1.00 | 21.52 | A | C |
| ATOM | 1053 | CB | PHE | | 151 | 34.378 | 98.671 | −70.209 | 1.00 | 20.18 | A | C |
| ATOM | 1054 | CG | PHE | | 151 | 34.947 | 97.339 | −69.859 | 1.00 | 23.04 | A | C |
| ATOM | 1055 | CD1 | PHE | | 151 | 36.328 | 97.171 | −69.706 | 1.00 | 22.27 | A | C |
| ATOM | 1056 | CD2 | PHE | | 151 | 34.126 | 96.250 | −69.715 | 1.00 | 21.27 | A | C |
| ATOM | 1057 | CE1 | PHE | | 151 | 36.845 | 95.937 | −69.417 | 1.00 | 23.27 | A | C |
| ATOM | 1058 | CE2 | PHE | | 151 | 34.649 | 95.000 | −69.420 | 1.00 | 22.33 | A | C |
| ATOM | 1059 | CZ | PHE | | 151 | 36.003 | 94.840 | −69.272 | 1.00 | 23.79 | A | C |
| ATOM | 1060 | C | PHE | | 151 | 33.704 | 100.291 | −71.965 | 1.00 | 21.36 | A | C |
| ATOM | 1061 | O | PHE | | 151 | 34.202 | 101.382 | −71.680 | 1.00 | 21.59 | A | O |
| ATOM | 1062 | N | THR | | 152 | 32.524 | 100.172 | −72.556 | 1.00 | 22.60 | A | N |
| ATOM | 1063 | CA | THR | | 152 | 31.688 | 101.344 | −72.812 | 1.00 | 23.33 | A | C |
| ATOM | 1064 | CB | THR | | 152 | 31.777 | 101.878 | −74.285 | 1.00 | 24.44 | A | C |
| ATOM | 1065 | OG1 | THR | | 152 | 31.179 | 100.904 | −75.139 | 1.00 | 23.16 | A | O |
| ATOM | 1066 | CG2 | THR | | 152 | 33.247 | 102.161 | −74.721 | 1.00 | 24.50 | A | C |
| ATOM | 1067 | C | THR | | 152 | 30.247 | 100.893 | −72.594 | 1.00 | 22.81 | A | C |
| ATOM | 1068 | O | THR | | 152 | 29.958 | 99.689 | −72.480 | 1.00 | 20.21 | A | O |
| ATOM | 1069 | N | ASP | | 153 | 29.337 | 101.865 | −72.545 | 1.00 | 25.30 | A | N |
| ATOM | 1070 | CA | ASP | | 153 | 27.928 | 101.537 | −72.366 | 1.00 | 28.11 | A | C |
| ATOM | 1071 | CB | ASP | | 153 | 27.101 | 102.814 | −72.357 | 1.00 | 31.85 | A | C |
| ATOM | 1072 | CG | ASP | | 153 | 26.841 | 103.311 | −70.969 | 1.00 | 37.63 | A | C |
| ATOM | 1073 | OD1 | ASP | | 153 | 27.756 | 103.219 | −70.108 | 1.00 | 39.38 | A | O |
| ATOM | 1074 | OD2 | ASP | | 153 | 25.707 | 103.808 | −70.749 | 1.00 | 41.99 | A | O |
| ATOM | 1075 | C | ASP | | 153 | 27.403 | 100.627 | −73.454 | 1.00 | 28.36 | A | C |
| ATOM | 1076 | O | ASP | | 153 | 26.428 | 99.888 | −73.237 | 1.00 | 29.95 | A | O |
| ATOM | 1077 | N | SER | | 154 | 28.019 | 100.665 | −74.643 | 1.00 | 27.09 | A | N |
| ATOM | 1078 | CA | SER | | 154 | 27.501 | 99.826 | −75.706 | 1.00 | 25.64 | A | C |
| ATOM | 1079 | CB | SER | | 154 | 27.748 | 100.487 | −77.077 | 1.00 | 27.69 | A | C |
| ATOM | 1080 | OG | SER | | 154 | 29.132 | 100.540 | −77.395 | 1.00 | 32.52 | A | O |
| ATOM | 1081 | C | SER | | 154 | 28.027 | 98.390 | −75.655 | 1.00 | 24.75 | A | C |
| ATOM | 1082 | O | SER | | 154 | 27.415 | 97.465 | −76.226 | 1.00 | 22.83 | A | O |
| ATOM | 1083 | N | GLY | | 155 | 29.114 | 98.181 | −74.906 | 1.00 | 21.12 | A | N |
| ATOM | 1084 | CA | GLY | | 155 | 29.662 | 96.836 | −74.821 | 1.00 | 20.72 | A | C |
| ATOM | 1085 | C | GLY | | 155 | 31.181 | 96.911 | −74.659 | 1.00 | 18.96 | A | C |
| ATOM | 1086 | O | GLY | | 155 | 31.702 | 97.956 | −74.291 | 1.00 | 18.55 | A | O |
| ATOM | 1087 | N | ILE | | 156 | 31.880 | 95.813 | −74.945 | 1.00 | 17.66 | A | N |
| ATOM | 1088 | CA | ILE | | 156 | 33.344 | 95.784 | −74.793 | 1.00 | 17.00 | A | C |
| ATOM | 1089 | CB | ILE | | 156 | 33.808 | 94.483 | −74.082 | 1.00 | 17.02 | A | C |
| ATOM | 1090 | CG2 | ILE | | 156 | 35.368 | 94.514 | −73.853 | 1.00 | 19.62 | A | C |
| ATOM | 1091 | CG1 | ILE | | 156 | 33.038 | 94.296 | −72.742 | 1.00 | 19.91 | A | C |
| ATOM | 1092 | CD1 | ILE | | 156 | 33.285 | 92.909 | −72.055 | 1.00 | 20.07 | A | C |
| ATOM | 1093 | C | ILE | | 156 | 33.972 | 95.810 | −76.189 | 1.00 | 16.59 | A | C |
| ATOM | 1094 | O | ILE | | 156 | 33.611 | 95.014 | −77.051 | 1.00 | 17.11 | A | O |
| ATOM | 1095 | N | HIS | | 157 | 34.944 | 96.690 | −76.394 | 1.00 | 18.12 | A | N |
| ATOM | 1096 | CA | HIS | | 157 | 35.593 | 96.802 | −77.690 | 1.00 | 16.12 | A | C |
| ATOM | 1097 | CB | HIS | | 157 | 35.611 | 98.269 | −78.138 | 1.00 | 17.10 | A | C |
| ATOM | 1098 | CG | HIS | | 157 | 34.284 | 98.978 | −78.109 | 1.00 | 18.76 | A | C |
| ATOM | 1099 | CD2 | HIS | | 157 | 33.538 | 99.449 | −77.071 | 1.00 | 18.91 | A | C |
| ATOM | 1100 | ND1 | HIS | | 157 | 33.706 | 99.483 | −79.253 | 1.00 | 17.73 | A | N |
| ATOM | 1101 | CE1 | HIS | | 157 | 32.674 | 100.255 | −78.921 | 1.00 | 20.15 | A | C |
| ATOM | 1102 | NE2 | HIS | | 157 | 32.551 | 100.249 | −77.605 | 1.00 | 17.69 | A | N |
| ATOM | 1103 | C | HIS | | 157 | 37.097 | 96.398 | −77.682 | 1.00 | 17.40 | A | C |
| ATOM | 1104 | O | HIS | | 157 | 37.754 | 96.413 | −76.635 | 1.00 | 17.71 | A | O |
| ATOM | 1105 | N | ILE | | 158 | 37.599 | 96.065 | −78.870 | 1.00 | 15.28 | A | N |
| ATOM | 1106 | CA | ILE | | 158 | 39.031 | 95.910 | −79.112 | 1.00 | 16.21 | A | C |
| ATOM | 1107 | CB | ILE | | 158 | 39.414 | 94.580 | −79.766 | 1.00 | 16.97 | A | C |
| ATOM | 1108 | CG2 | ILE | | 158 | 40.942 | 94.616 | −80.060 | 1.00 | 19.87 | A | C |
| ATOM | 1109 | CG1 | ILE | | 158 | 39.150 | 93.428 | −78.801 | 1.00 | 18.11 | A | C |
| ATOM | 1110 | CD1 | ILE | | 158 | 39.204 | 92.071 | −79.492 | 1.00 | 18.15 | A | C |
| ATOM | 1111 | C | ILE | | 158 | 39.143 | 97.044 | −80.169 | 1.00 | 16.56 | A | C |
| ATOM | 1112 | O | ILE | | 158 | 38.345 | 97.080 | −81.110 | 1.00 | 17.25 | A | O |
| ATOM | 1113 | N | ILE | | 159 | 40.074 | 97.979 | −79.999 | 1.00 | 15.75 | A | N |
| ATOM | 1114 | CA | ILE | | 159 | 40.190 | 99.109 | −80.916 | 1.00 | 15.79 | A | C |
| ATOM | 1115 | CB | ILE | | 159 | 40.069 | 100.469 | −80.161 | 1.00 | 15.18 | A | C |
| ATOM | 1116 | CG2 | ILE | | 159 | 40.215 | 101.640 | −81.108 | 1.00 | 14.17 | A | C |
| ATOM | 1117 | CG1 | ILE | | 159 | 38.756 | 100.522 | −79.417 | 1.00 | 18.67 | A | C |
| ATOM | 1118 | CD1 | ILE | | 159 | 38.670 | 101.700 | −78.379 | 1.00 | 16.07 | A | C |
| ATOM | 1119 | C | ILE | | 159 | 41.545 | 99.052 | −81.616 | 1.00 | 16.35 | A | C |
| ATOM | 1120 | O | ILE | | 159 | 42.551 | 98.754 | −80.991 | 1.00 | 17.23 | A | O |
| ATOM | 1121 | N | LEU | | 160 | 41.550 | 99.299 | −82.925 | 1.00 | 14.90 | A | N |
| ATOM | 1122 | CA | LEU | | 160 | 42.804 | 99.294 | −83.681 | 1.00 | 15.91 | A | C |
| ATOM | 1123 | CB | LEU | | 160 | 42.784 | 98.243 | −84.806 | 1.00 | 14.83 | A | C |
| ATOM | 1124 | CG | LEU | | 160 | 44.001 | 98.257 | −85.755 | 1.00 | 17.97 | A | C |
| ATOM | 1125 | CD1 | LEU | | 160 | 45.243 | 97.720 | −85.026 | 1.00 | 16.47 | A | C |
| ATOM | 1126 | CD2 | LEU | | 160 | 43.756 | 97.401 | −86.975 | 1.00 | 19.42 | A | C |
| ATOM | 1127 | C | LEU | | 160 | 42.961 | 100.683 | −84.303 | 1.00 | 15.46 | A | C |
| ATOM | 1128 | O | LEU | | 160 | 42.118 | 101.120 | −85.062 | 1.00 | 17.21 | A | O |
| ATOM | 1129 | N | ARG | | 161 | 44.020 | 101.373 | −83.953 | 1.00 | 17.32 | A | N |
| ATOM | 1130 | CA | ARG | | 161 | 44.227 | 102.692 | −84.540 | 1.00 | 18.67 | A | C |
| ATOM | 1131 | CB | ARG | | 161 | 45.013 | 103.612 | −83.603 | 1.00 | 18.01 | A | C |
| ATOM | 1132 | CG | ARG | | 161 | 45.354 | 104.930 | −84.330 | 1.00 | 21.10 | A | C |
| ATOM | 1133 | CD | ARG | | 161 | 46.032 | 105.994 | −83.431 | 1.00 | 24.02 | A | C |
| ATOM | 1134 | NE | ARG | | 161 | 46.346 | 107.148 | −84.271 | 1.00 | 26.05 | A | N |
| ATOM | 1135 | CZ | ARG | | 161 | 47.196 | 108.115 | −83.934 | 1.00 | 28.94 | A | C |
| ATOM | 1136 | NH1 | ARG | | 161 | 47.809 | 108.079 | −82.747 | 1.00 | 29.33 | A | N |
| ATOM | 1137 | NH2 | ARG | | 161 | 47.482 | 109.085 | −84.808 | 1.00 | 27.52 | A | N |
| ATOM | 1138 | C | ARG | | 161 | 44.952 | 102.491 | −85.879 | 1.00 | 18.85 | A | C |
| ATOM | 1139 | O | ARG | | 161 | 46.046 | 101.916 | −85.936 | 1.00 | 19.13 | A | O |
| ATOM | 1140 | N | THR | | 162 | 44.307 | 102.924 | −86.952 | 1.00 | 19.16 | A | N |
| ATOM | 1141 | CA | THR | | 162 | 44.877 | 102.761 | −88.286 | 1.00 | 21.01 | A | C |
| ATOM | 1142 | CB | THR | | 162 | 43.807 | 102.302 | −89.256 | 1.00 | 20.38 | A | C |
| ATOM | 1143 | OG1 | THR | | 162 | 42.725 | 103.237 | −89.245 | 1.00 | 21.90 | A | O |
| ATOM | 1144 | CG2 | THR | | 162 | 43.302 | 100.884 | −88.851 | 1.00 | 19.88 | A | C |
| ATOM | 1145 | C | THR | | 162 | 45.522 | 104.046 | −88.837 | 1.00 | 23.01 | A | C |
| ATOM | 1146 | O | THR | | 162 | 46.309 | 103.965 | −89.766 | 1.00 | 22.61 | A | O |
| ATOM | 1147 | N | GLU | | 163 | 45.167 | 105.206 | −88.278 | 1.00 | 23.69 | A | N |
| ATOM | 1148 | CA | GLU | | 163 | 45.744 | 106.497 | −88.709 | 1.00 | 26.00 | A | C |
| ATOM | 1149 | CB | GLU | | 163 | 44.930 | 107.128 | −89.860 | 1.00 | 27.09 | A | C |
| ATOM | 1150 | CG | GLU | | 163 | 44.841 | 106.304 | −91.128 | 1.00 | 28.74 | A | C |
| ATOM | 1151 | CD | GLU | | 163 | 44.005 | 106.980 | −92.214 | 1.00 | 31.06 | A | C |
| ATOM | 1152 | OE1 | GLU | | 163 | 43.536 | 108.124 | −91.994 | 1.00 | 33.77 | A | O |
| ATOM | 1153 | OE2 | GLU | | 163 | 43.814 | 106.364 | −93.282 | 1.00 | 34.04 | A | O |
| ATOM | 1154 | C | GLU | | 163 | 45.721 | 107.480 | −87.555 | 1.00 | 26.50 | A | C |
| ATOM | 1155 | O | GLU | | 163 | 44.872 | 107.275 | −86.663 | 1.00 | 25.73 | A | O |
| ATOM | 1156 | OXT | GLU | | 163 | 46.510 | 108.472 | −87.579 | 1.00 | 27.66 | A | O |
| TER | 1157 | | GLU | | 163 | | | | | | A | |
| ATOM | 1158 | OH2 | TIP | S | 1 | 48.082 | 101.427 | −75.587 | 1.00 | 18.04 | | O |
| ATOM | 1159 | OH2 | TIP | S | 2 | 51.585 | 89.916 | −81.211 | 1.00 | 21.10 | | O |
| ATOM | 1160 | OH2 | TIP | S | 3 | 51.859 | 92.089 | −76.864 | 1.00 | 21.56 | | O |
| ATOM | 1161 | OH2 | TIP | S | 4 | 33.038 | 96.453 | −85.963 | 1.00 | 17.76 | | O |
| ATOM | 1162 | OH2 | TIP | S | 5 | 49.809 | 103.573 | −76.650 | 1.00 | 19.97 | | O |
| ATOM | 1163 | OH2 | TIP | S | 6 | 42.201 | 100.433 | −67.302 | 1.00 | 19.76 | | O |
| ATOM | 1164 | OH2 | TIP | S | 7 | 29.972 | 91.630 | −91.548 | 1.00 | 21.62 | | O |
| ATOM | 1165 | OH2 | TIP | S | 8 | 40.318 | 101.816 | −89.576 | 1.00 | 19.74 | | O |
| ATOM | 1166 | OH2 | TIP | S | 9 | 26.286 | 88.424 | −80.928 | 1.00 | 25.66 | | O |
| ATOM | 1167 | OH2 | TIP | S | 10 | 39.431 | 97.561 | −90.394 | 1.00 | 22.73 | | O |
| ATOM | 1168 | OH2 | TIP | S | 11 | 47.173 | 89.684 | −86.326 | 1.00 | 16.74 | | O |
| ATOM | 1169 | OH2 | TIP | S | 12 | 37.748 | 100.288 | −70.225 | 1.00 | 20.44 | | O |
| ATOM | 1170 | OH2 | TIP | S | 13 | 35.319 | 94.118 | −96.397 | 1.00 | 28.89 | | O |
| ATOM | 1171 | OH2 | TIP | S | 14 | 45.823 | 93.900 | −70.890 | 1.00 | 32.12 | | O |
| ATOM | 1172 | OH2 | TIP | S | 15 | 24.392 | 86.549 | −76.670 | 1.00 | 36.77 | | O |
| ATOM | 1173 | OH2 | TIP | S | 16 | 36.930 | 84.348 | −78.407 | 1.00 | 24.07 | | O |
| ATOM | 1174 | OH2 | TIP | S | 17 | 30.444 | 104.610 | −72.507 | 1.00 | 33.01 | | O |
| ATOM | 1175 | OH2 | TIP | S | 18 | 35.656 | 111.545 | −79.929 | 1.00 | 34.16 | | O |
| ATOM | 1176 | OH2 | TIP | S | 19 | 49.491 | 90.772 | −71.572 | 1.00 | 22.03 | | O |
| ATOM | 1177 | OH2 | TIP | S | 20 | 33.500 | 83.562 | −81.832 | 1.00 | 31.26 | | O |
| ATOM | 1178 | OH2 | TIP | S | 21 | 33.951 | 93.386 | −93.865 | 1.00 | 22.89 | | O |
| ATOM | 1179 | OH2 | TIP | S | 22 | 29.775 | 89.263 | −80.786 | 1.00 | 22.40 | | O |
| ATOM | 1180 | OH2 | TIP | S | 23 | 31.250 | 87.067 | −80.332 | 1.00 | 29.99 | | O |
| ATOM | 1181 | OH2 | TIP | S | 24 | 14.398 | 93.475 | −82.021 | 1.00 | 50.82 | | O |
| ATOM | 1182 | OH2 | TIP | S | 25 | 43.747 | 88.370 | −74.291 | 1.00 | 23.50 | | O |
| ATOM | 1183 | OH2 | TIP | S | 26 | 42.882 | 90.738 | −73.315 | 1.00 | 31.29 | | O |
| ATOM | 1184 | OH2 | TIP | S | 27 | 32.177 | 83.374 | −88.949 | 1.00 | 36.46 | | O |
| ATOM | 1185 | OH2 | TIP | S | 28 | 37.581 | 91.106 | −93.529 | 1.00 | 49.86 | | O |
| ATOM | 1186 | OH2 | TIP | S | 29 | 30.669 | 97.024 | −71.398 | 1.00 | 31.36 | | O |
| ATOM | 1187 | OH2 | TIP | S | 30 | 39.488 | 106.765 | −70.207 | 1.00 | 42.47 | | O |
| ATOM | 1188 | OH2 | TIP | S | 31 | 59.907 | 93.177 | −70.669 | 1.00 | 25.62 | | O |
| ATOM | 1189 | OH2 | TIP | S | 32 | 27.485 | 86.442 | −87.274 | 1.00 | 35.47 | | O |
| ATOM | 1190 | OH2 | TIP | S | 33 | 43.516 | 109.696 | −74.501 | 1.00 | 27.08 | | O |
| ATOM | 1191 | OH2 | TIP | S | 34 | 40.284 | 115.890 | −81.404 | 1.00 | 41.78 | | O |
| ATOM | 1192 | OH2 | TIP | S | 35 | 62.200 | 97.097 | −70.936 | 1.00 | 44.03 | | O |
| ATOM | 1193 | OH2 | TIP | S | 36 | 50.803 | 88.917 | −64.686 | 1.00 | 32.48 | | O |
| ATOM | 1194 | OH2 | TIP | S | 37 | 25.215 | 98.897 | −85.264 | 1.00 | 34.24 | | O |
| ATOM | 1195 | OH2 | TIP | S | 38 | 48.708 | 102.884 | −61.798 | 1.00 | 33.13 | | O |
| ATOM | 1196 | OH2 | TIP | S | 39 | 35.694 | 102.146 | −94.826 | 1.00 | 35.27 | | O |
| ATOM | 1197 | OH2 | TIP | S | 40 | 17.845 | 86.830 | −80.637 | 1.00 | 37.49 | | O |
| ATOM | 1198 | OH2 | TIP | S | 41 | 24.535 | 91.152 | −92.236 | 1.00 | 27.61 | | O |
| ATOM | 1199 | OH2 | TIP | S | 42 | 46.829 | 99.932 | −88.480 | 1.00 | 45.14 | | O |
| ATOM | 1200 | OH2 | TIP | S | 43 | 62.395 | 91.861 | −66.549 | 1.00 | 45.59 | | O |
| ATOM | 1201 | OH2 | TIP | S | 44 | 60.573 | 90.712 | −72.271 | 1.00 | 31.99 | | O |
| ATOM | 1202 | OH2 | TIP | S | 45 | 40.578 | 95.056 | −70.054 | 1.00 | 46.81 | | O |
| ATOM | 1203 | OH2 | TIP | S | 46 | 49.074 | 95.085 | −64.867 | 1.00 | 35.64 | | O |
| ATOM | 1204 | OH2 | TIP | S | 47 | 40.835 | 96.865 | −94.234 | 1.00 | 44.13 | | O |
| ATOM | 1205 | OH2 | TIP | S | 48 | 24.594 | 99.044 | −77.314 | 1.00 | 41.88 | | O |
| ATOM | 1206 | OH2 | TIP | S | 49 | 41.930 | 97.181 | −91.862 | 1.00 | 50.35 | | O |
| ATOM | 1207 | OH2 | TIP | S | 50 | 60.648 | 82.708 | −64.686 | 1.00 | 47.29 | | O |
| ATOM | 1208 | OH2 | TIP | S | 51 | 32.360 | 83.711 | −73.398 | 1.00 | 38.49 | | O |
| ATOM | 1209 | OH2 | TIP | S | 52 | 48.777 | 108.478 | −79.503 | 1.00 | 42.78 | | O |
| ATOM | 1210 | OH2 | TIP | S | 53 | 49.840 | 106.726 | −75.333 | 1.00 | 35.93 | | O |
| ATOM | 1211 | OH2 | TIP | S | 54 | 38.881 | 83.687 | −73.492 | 1.00 | 32.30 | | O |
| ATOM | 1212 | OH2 | TIP | S | 55 | 45.653 | 110.825 | −87.456 | 1.00 | 41.44 | | O |
| ATOM | 1213 | OH2 | TIP | S | 56 | 24.533 | 95.593 | −85.731 | 1.00 | 34.34 | | O |
| ATOM | 1214 | OH2 | TIP | S | 57 | 20.711 | 91.253 | −89.187 | 1.00 | 38.38 | | O |
| ATOM | 1215 | OH2 | TIP | S | 58 | 54.487 | 91.949 | −76.096 | 1.00 | 24.32 | | O |
| ATOM | 1216 | OH2 | TIP | S | 59 | 56.957 | 85.342 | −72.648 | 1.00 | 24.26 | | O |
| ATOM | 1217 | OH2 | TIP | S | 60 | 48.492 | 96.151 | −83.544 | 1.00 | 28.10 | | O |
| ATOM | 1218 | OH2 | TIP | S | 61 | 49.378 | 97.039 | −80.336 | 1.00 | 30.72 | | O |
| ATOM | 1219 | OH2 | TIP | S | 62 | 50.809 | 97.992 | −77.160 | 1.00 | 36.73 | | O |
| ATOM | 1220 | OH2 | TIP | S | 63 | 39.546 | 104.486 | −72.141 | 1.00 | 26.60 | | O |
| ATOM | 1221 | OH2 | TIP | S | 64 | 41.331 | 101.384 | −92.014 | 1.00 | 31.75 | | O |
| ATOM | 1222 | OH2 | TIP | S | 65 | 54.905 | 96.001 | −78.802 | 1.00 | 49.60 | | O |
| ATOM | 1223 | OH2 | TIP | S | 66 | 42.906 | 110.367 | −71.323 | 1.00 | 37.82 | | O |
| ATOM | 1224 | OH2 | TIP | S | 67 | 38.157 | 101.156 | −96.112 | 1.00 | 41.06 | | O |
| ATOM | 1225 | OH2 | TIP | S | 68 | 50.686 | 92.674 | −82.160 | 1.00 | 38.59 | | O |
| ATOM | 1226 | OH2 | TIP | S | 69 | 40.110 | 90.150 | −93.660 | 1.00 | 47.25 | | O |
| ATOM | 1227 | OH2 | TIP | S | 70 | 51.482 | 87.184 | −71.081 | 1.00 | 37.17 | | O |
| ATOM | 1228 | OH2 | TIP | S | 71 | 43.118 | 110.363 | −89.770 | 1.00 | 42.27 | | O |
| ATOM | 1229 | OH2 | TIP | S | 72 | 53.882 | 101.629 | −81.807 | 1.00 | 30.94 | | O |
| ATOM | 1230 | OH2 | TIP | S | 73 | 31.345 | 105.239 | −69.760 | 1.00 | 48.87 | | O |
| ATOM | 1231 | OH2 | TIP | S | 74 | 25.941 | 85.213 | −81.998 | 1.00 | 61.06 | | O |
| ATOM | 1232 | OH2 | TIP | S | 75 | 47.139 | 108.628 | −67.165 | 1.00 | 47.07 | | O |
| ATOM | 1233 | OH2 | TIP | S | 76 | 36.956 | 82.906 | −76.340 | 1.00 | 30.49 | | O |
| ATOM | 1234 | OH2 | TIP | S | 77 | 44.646 | 86.956 | −72.024 | 1.00 | 47.98 | | O |
| ATOM | 1235 | OH2 | TIP | S | 78 | 50.725 | 91.614 | −63.564 | 1.00 | 35.57 | | O |
| ATOM | 1236 | OH2 | TIP | S | 79 | 21.998 | 92.127 | −91.486 | 1.00 | 47.44 | | O |
| ATOM | 1237 | OH2 | TIP | S | 80 | 47.266 | 87.896 | −88.765 | 1.00 | 40.08 | | O |
| ATOM | 1238 | OH2 | TIP | S | 81 | 24.525 | 97.089 | −83.052 | 1.00 | 36.62 | | O |
| ATOM | 1239 | OH2 | TIP | S | 82 | 25.821 | 91.573 | −68.249 | 1.00 | 41.99 | | O |
| ATOM | 1240 | OH2 | TIP | S | 83 | 34.713 | 82.268 | −74.538 | 1.00 | 45.42 | | O |
| ATOM | 1241 | OH2 | TIP | S | 84 | 50.005 | 95.359 | −62.322 | 1.00 | 37.75 | | O |
| ATOM | 1242 | OH2 | TIP | S | 85 | 42.153 | 85.977 | −90.682 | 1.00 | 43.14 | | O |
| ATOM | 1243 | OH2 | TIP | S | 86 | 18.505 | 86.793 | −83.931 | 1.00 | 42.01 | | O |
| ATOM | 1244 | OH2 | TIP | S | 87 | 34.355 | 108.946 | −88.886 | 1.00 | 40.34 | | O |
| ATOM | 1245 | OH2 | TIP | S | 88 | 53.221 | 86.159 | −68.264 | 1.00 | 41.36 | | O |
| ATOM | 1246 | OH2 | TIP | S | 89 | 19.204 | 90.567 | −73.202 | 1.00 | 45.21 | | O |
| ATOM | 1247 | OH2 | TIP | S | 90 | 33.526 | 112.151 | −76.169 | 1.00 | 47.42 | | O |
| ATOM | 1248 | OH2 | TIP | S | 91 | 39.849 | 88.806 | −68.078 | 1.00 | 40.75 | | O |
| ATOM | 1249 | OH2 | TIP | S | 92 | 45.310 | 81.899 | −79.875 | 1.00 | 42.32 | | O |
| ATOM | 1250 | OH2 | TIP | S | 93 | 28.805 | 84.754 | −66.693 | 1.00 | 41.71 | | O |
| ATOM | 1251 | OH2 | TIP | S | 94 | 46.682 | 94.036 | −83.802 | 1.00 | 21.76 | | O |
| ATOM | 1252 | OH2 | TIP | S | 95 | 40.626 | 91.047 | −69.447 | 1.00 | 28.31 | | O |
| ATOM | 1253 | OH2 | TIP | S | 96 | 49.160 | 93.687 | −80.396 | 1.00 | 33.38 | | O |
| ATOM | 1254 | OH2 | TIP | S | 97 | 45.166 | 89.424 | −70.525 | 1.00 | 44.15 | | O |
| ATOM | 1255 | OH2 | TIP | S | 98 | 46.654 | 85.443 | −72.900 | 1.00 | 50.39 | | O |
| ATOM | 1256 | OH2 | TIP | S | 99 | 41.247 | 108.915 | −92.938 | 1.00 | 37.86 | | O |
| ATOM | 1257 | OH2 | TIP | S | 100 | 32.089 | 112.917 | −73.986 | 1.00 | 34.52 | | O |
| ATOM | 1258 | OH2 | TIP | S | 101 | 51.281 | 107.764 | −83.744 | 1.00 | 46.49 | | O |
| ATOM | 1259 | OH2 | TIP | S | 102 | 37.105 | 87.260 | −92.888 | 1.00 | 47.81 | | O |
| ATOM | 1260 | OH2 | TIP | S | 103 | 35.387 | 82.572 | −80.116 | 1.00 | 47.95 | | O |
| ATOM | 1261 | OH2 | TIP | S | 104 | 38.414 | 83.287 | −80.796 | 1.00 | 38.64 | | O |
| ATOM | 1262 | OH2 | TIP | S | 105 | 25.283 | 100.226 | −80.660 | 1.00 | 42.56 | | O |
| ATOM | 1263 | OH2 | TIP | S | 106 | 43.535 | 91.874 | −71.251 | 1.00 | 37.97 | | O |
| ATOM | 1264 | OH2 | TIP | S | 107 | 49.646 | 110.064 | −82.866 | 1.00 | 44.76 | | O |
| ATOM | 1265 | OH2 | TIP | S | 108 | 45.929 | 81.087 | −89.510 | 1.00 | 54.16 | | O |
| ATOM | 1266 | OH2 | TIP | S | 109 | 54.977 | 101.974 | −63.060 | 1.00 | 45.43 | | O |
| ATOM | 1267 | OH2 | TIP | S | 110 | 40.987 | 81.884 | −75.639 | 1.00 | 46.24 | | O |
| ATOM | 1268 | OH2 | TIP | S | 111 | 52.104 | 94.013 | −78.913 | 1.00 | 51.64 | | O |
| ATOM | 1269 | OH2 | TIP | S | 112 | 39.338 | 84.754 | −70.979 | 1.00 | 42.99 | | O |
| ATOM | 1270 | OH2 | TIP | S | 113 | 43.616 | 99.385 | −92.171 | 1.00 | 43.66 | | O |
| ATOM | 1271 | OH2 | TIP | S | 114 | 36.018 | 114.263 | −74.511 | 1.00 | 30.72 | | O |
| ATOM | 1272 | OH2 | TIP | S | 115 | 45.847 | 94.011 | −85.968 | 1.00 | 40.49 | | O |
| ATOM | 1273 | OH2 | TIP | S | 116 | 46.016 | 110.954 | −71.456 | 1.00 | 47.81 | | O |
| ATOM | 1274 | OH2 | TIP | S | 117 | 44.513 | 114.302 | −72.901 | 1.00 | 53.90 | | O |
| ATOM | 1275 | OH2 | TIP | S | 118 | 50.962 | 83.279 | −62.390 | 1.00 | 61.22 | | O |
| ATOM | 1276 | OH2 | TIP | S | 119 | 47.020 | 93.741 | −67.854 | 1.00 | 49.47 | | O |
| ATOM | 1277 | OH2 | TIP | S | 120 | 28.311 | 110.301 | −77.416 | 1.00 | 51.56 | | O |
| ATOM | 1278 | OH2 | TIP | S | 121 | 40.484 | 112.927 | −71.586 | 1.00 | 51.28 | | O |
| ATOM | 1279 | OH2 | TIP | S | 122 | 47.079 | 100.887 | −90.865 | 1.00 | 42.60 | | O |
| ATOM | 1280 | OH2 | TIP | S | 123 | 18.931 | 89.924 | −69.830 | 1.00 | 65.48 | | O |
| ATOM | 1281 | OH2 | TIP | S | 124 | 30.774 | 109.351 | −85.188 | 1.00 | 58.50 | | O |
| ATOM | 1282 | OH2 | TIP | S | 125 | 30.468 | 102.147 | −91.685 | 1.00 | 61.00 | | O |
| ATOM | 1283 | OH2 | TIP | S | 126 | 51.309 | 98.056 | −61.812 | 1.00 | 49.51 | | O |
| ATOM | 1284 | OH2 | TIP | S | 127 | 36.974 | 80.645 | −81.661 | 1.00 | 47.95 | | O |
| ATOM | 1285 | OH2 | TIP | S | 128 | 55.630 | 96.621 | −60.667 | 1.00 | 54.25 | | O |
| ATOM | 1286 | OH2 | TIP | S | 129 | 25.808 | 99.363 | −70.049 | 1.00 | 58.33 | | O |
| ATOM | 1287 | OH2 | TIP | S | 130 | 20.718 | 87.915 | −70.182 | 1.00 | 53.58 | | O |
| ATOM | 1288 | OH2 | TIP | S | 131 | 14.944 | 91.204 | −71.966 | 1.00 | 49.00 | | O |
| ATOM | 1289 | OH2 | TIP | S | 132 | 30.706 | 114.267 | −88.445 | 1.00 | 46.32 | | O |
| ATOM | 1290 | OH2 | TIP | S | 133 | 25.963 | 102.889 | −82.324 | 1.00 | 46.73 | | O |
| ATOM | 1291 | OH2 | TIP | S | 134 | 27.251 | 104.246 | −80.470 | 1.00 | 55.57 | | O |
| ATOM | 1292 | OH2 | TIP | S | 135 | 11.818 | 92.001 | −85.633 | 1.00 | 56.75 | | O |
| ATOM | 1293 | OH2 | TIP | S | 136 | 58.353 | 106.515 | −75.598 | 1.00 | 54.23 | | O |
| ATOM | 1294 | OH2 | TIP | S | 137 | 51.150 | 102.437 | −60.510 | 1.00 | 64.21 | | O |
| ATOM | 1295 | OH2 | TIP | S | 138 | 23.732 | 82.030 | −82.878 | 1.00 | 45.88 | | O |
| ATOM | 1296 | OH2 | TIP | S | 139 | 41.420 | 87.391 | −66.939 | 1.00 | 64.87 | | O |
| ATOM | 1297 | OH2 | TIP | S | 140 | 52.198 | 99.844 | −80.990 | 1.00 | 37.17 | | O |
| ATOM | 1298 | OH2 | TIP | S | 141 | 19.795 | 92.125 | −67.847 | 1.00 | 74.10 | | O |
| ATOM | 1299 | OH2 | TIP | S | 142 | 25.400 | 98.836 | −66.728 | 1.00 | 61.76 | | O |
| ATOM | 1300 | OH2 | TIP | S | 143 | 27.980 | 98.080 | −69.199 | 1.00 | 53.46 | | O |
| ATOM | 1301 | OH2 | TIP | S | 144 | 62.351 | 86.545 | −63.345 | 1.00 | 67.01 | | O |
| ATOM | 1302 | OH2 | TIP | S | 145 | 29.019 | 93.103 | −64.478 | 1.00 | 61.40 | | O |
| ATOM | 1303 | OH2 | TIP | S | 146 | 33.534 | 91.535 | −66.206 | 1.00 | 48.23 | | O |
| ATOM | 1304 | OH2 | TIP | S | 147 | 31.924 | 96.329 | −66.049 | 1.00 | 51.07 | | O |
| ATOM | 1305 | OH2 | TIP | S | 148 | 52.297 | 105.197 | −76.211 | 1.00 | 47.94 | | O |
| ATOM | 1306 | OH2 | TIP | S | 149 | 64.486 | 89.161 | −62.988 | 1.00 | 57.59 | | O |
| ATOM | 1307 | OH2 | TIP | S | 150 | 30.423 | 93.203 | −61.399 | 1.00 | 74.96 | | O |
| ATOM | 1308 | OH2 | TIP | S | 151 | 48.960 | 108.294 | −88.732 | 1.00 | 54.04 | | O |
| ATOM | 1309 | OH2 | TIP | S | 152 | 29.297 | 112.506 | −77.035 | 1.00 | 57.06 | | O |
| ATOM | 1310 | OH2 | TIP | S | 153 | 25.147 | 83.913 | −79.056 | 1.00 | 63.36 | | O |
| ATOM | 1311 | OH2 | TIP | S | 154 | 53.799 | 100.274 | −61.424 | 1.00 | 83.07 | | O |
| ATOM | 1312 | OH2 | TIP | S | 155 | 54.765 | 100.624 | −79.708 | 1.00 | 53.71 | | O |
| ATOM | 1313 | OH2 | TIP | S | 156 | 64.045 | 98.729 | −71.705 | 1.00 | 59.95 | | O |
| ATOM | 1314 | OH2 | TIP | S | 157 | 28.776 | 102.323 | −88.614 | 1.00 | 61.81 | | O |
| ATOM | 1315 | OH2 | TIP | S | 158 | 38.708 | 93.225 | −66.167 | 1.00 | 51.98 | | O |
| ATOM | 1316 | OH2 | TIP | S | 159 | 50.807 | 108.034 | −68.758 | 1.00 | 51.07 | | O |
| ATOM | 1317 | OH2 | TIP | S | 160 | 57.116 | 108.308 | −78.580 | 1.00 | 75.81 | | O |
| ATOM | 1318 | OH2 | TIP | S | 161 | 42.409 | 116.570 | −74.592 | 1.00 | 64.54 | | O |
| ATOM | 1319 | OH2 | TIP | S | 162 | 37.989 | 117.914 | −91.780 | 1.00 | 72.93 | | O |
| TER | 1320 | | TIP | S | 162 | | | | | | | |
| ATOM | 1321 | O1 | PGB | C | 180 | 48.514 | 90.887 | −68.146 | 1.00 | 42.43 | O | |
| ATOM | 1322 | C2 | PGB | C | 180 | 49.510 | 90.371 | −67.356 | 1.00 | 41.14 | C | |
| ATOM | 1323 | C3 | PGB | C | 180 | 50.426 | 89.386 | −68.116 | 1.00 | 39.51 | C | |
| ATOM | 1324 | O4 | PGB | C | 180 | 51.005 | 89.972 | −69.177 | 1.00 | 36.54 | O | |
| ATOM | 1325 | C5 | PGB | C | 180 | 52.325 | 89.918 | −69.146 | 1.00 | 30.79 | C | |
| ATOM | 1326 | C6 | PGB | C | 180 | 52.853 | 90.612 | −70.393 | 1.00 | 27.83 | C | |
| ATOM | 1327 | O7 | PGB | C | 180 | 52.334 | 89.997 | −71.488 | 1.00 | 25.49 | O | |
| ATOM | 1328 | C8 | PGB | C | 180 | 52.770 | 90.596 | −72.619 | 1.00 | 23.93 | C | |
| ATOM | 1329 | C9 | PGB | C | 180 | 52.165 | 89.885 | −73.840 | 1.00 | 25.97 | C | |
| ATOM | 1330 | O10 | PGB | C | 180 | 50.893 | 89.439 | −73.566 | 1.00 | 23.28 | O | |
| ATOM | 1331 | C11 | PGB | C | 180 | 50.349 | 88.795 | −74.637 | 1.00 | 19.63 | C | |
| ATOM | 1332 | C12 | PGB | C | 180 | 48.832 | 88.644 | −74.414 | 1.00 | 21.63 | C | |
| ATOM | 1333 | O13 | PGB | C | 180 | 48.212 | 89.869 | −74.455 | 1.00 | 21.22 | O | |
| ATOM | 1334 | C14 | PGB | C | 180 | 46.945 | 89.707 | −74.015 | 1.00 | 20.73 | C | |
| ATOM | 1335 | C15 | PGB | C | 180 | 46.266 | 91.096 | −73.745 | 1.00 | 20.29 | C | |
| ATOM | 1336 | O16 | PGB | C | 180 | 46.507 | 91.948 | −74.769 | 1.00 | 20.00 | O | |
| ATOM | 1337 | C17 | PGB | C | 180 | 45.890 | 93.087 | −74.528 | 1.00 | 26.07 | C | |
| ATOM | 1338 | C18 | PGB | C | 180 | 46.103 | 94.124 | −75.648 | 1.00 | 26.71 | C | |
| ATOM | 1339 | O19 | PGB | C | 180 | 47.401 | 94.435 | −75.676 | 1.00 | 28.38 | O | |
| ATOM | 1340 | C20 | PGB | C | 180 | 47.625 | 95.037 | −76.821 | 1.00 | 34.40 | C | |
| ATOM | 1341 | C21 | PGB | C | 180 | 48.787 | 95.663 | −77.584 | 1.00 | 35.84 | C | |
| ATOM | 1342 | O22 | PGB | C | 180 | 49.765 | 94.711 | −77.691 | 1.00 | 41.19 | O | |
| TER | 1343 | | PGB | C | 180 | | | | | | | |
| ATOM | 1344 | C2 | 420 | D | 181 | 29.409 | 102.512 | −83.178 | 1.00 | 44.95 | C | |
| ATOM | 1345 | C3 | 420 | D | 181 | 29.228 | 101.649 | −82.070 | 1.00 | 43.04 | C | |
| ATOM | 1346 | N11 | 420 | D | 181 | 28.535 | 100.466 | −82.231 | 1.00 | 40.67 | N | |
| ATOM | 1347 | C4 | 420 | D | 181 | 29.768 | 102.027 | −80.794 | 1.00 | 43.30 | C | |
| ATOM | 1348 | O10 | 420 | D | 181 | 29.570 | 101.171 | −79.733 | 1.00 | 41.69 | O | |
| ATOM | 1349 | C5 | 420 | D | 181 | 30.483 | 103.278 | −80.644 | 1.00 | 44.77 | C | |
| ATOM | 1350 | C7 | 420 | D | 181 | 31.081 | 103.769 | −79.309 | 1.00 | 45.52 | C | |
| ATOM | 1351 | O9 | 420 | D | 181 | 30.968 | 103.078 | −78.306 | 1.00 | 45.53 | O | |
| ATOM | 1352 | O8 | 420 | D | 181 | 31.723 | 104.974 | −79.240 | 1.00 | 46.59 | O | |
| ATOM | 1353 | C6 | 420 | D | 181 | 30.637 | 104.105 | −81.783 | 1.00 | 45.63 | C | |
| ATOM | 1354 | C1 | 420 | D | 181 | 30.108 | 103.744 | −83.059 | 1.00 | 46.86 | C | |
| ATOM | 1355 | N14 | 420 | D | 181 | 30.265 | 104.567 | −84.154 | 1.00 | 48.36 | N | |
| ATOM | 1356 | O16 | 420 | D | 181 | 29.742 | 104.224 | −85.416 | 1.00 | 51.77 | O | |
| ATOM | 1357 | O15 | 420 | D | 181 | 30.860 | 105.631 | −84.071 | 1.00 | 51.52 | O | |
| TER | 1358 | | 420 | D | 181 | | | | | | | |
| ATOM | 1359 | O1 | PGE | E | 182 | 30.158 | 82.656 | −82.944 | 1.00 | 78.51 | O | |
| ATOM | 1360 | C2 | PGE | E | 182 | 29.535 | 83.742 | −82.406 | 1.00 | 79.19 | C | |
| ATOM | 1361 | C3 | PGE | E | 182 | 30.104 | 84.115 | −81.019 | 1.00 | 79.22 | C | |
| ATOM | 1362 | O4 | PGE | E | 182 | 29.250 | 83.756 | −80.047 | 1.00 | 80.28 | O | |
| ATOM | 1363 | C5 | PGE | E | 182 | 29.069 | 84.697 | −79.111 | 1.00 | 79.37 | C | |
| ATOM | 1364 | C6 | PGE | E | 182 | 28.063 | 84.198 | −78.029 | 1.00 | 78.19 | C | |
| ATOM | 1365 | O7 | PGE | E | 182 | 28.679 | 84.163 | −76.813 | 1.00 | 77.95 | O | |
| ATOM | 1366 | C8 | PGE | E | 182 | 27.878 | 83.734 | −75.818 | 1.00 | 77.77 | C | |
| ATOM | 1367 | C9 | PGE | E | 182 | 28.655 | 83.708 | −74.470 | 1.00 | 77.35 | C | |
| ATOM | 1368 | O10 | PGE | E | 182 | 29.733 | 82.863 | −74.535 | 1.00 | 76.68 | O | |

Pin1 R14A/Pin-221 Interactions

The crystal structure of the PIN-221/R14A co-complex (the coordinate set is set forth in Table 4), shows a covalent interaction of Cys113 with PIN-221 and hydrogen bond interactions with Ser114. Other interactions involve van der Waals contacts with Ser154 and Ser171. A co-solvent molecule of N-methylpyrroleidione was also present, making a hydrogen bond with Gln131 and van der Waals contacts with Met130 and His157.

TABLE 4
Set of atomic coordinates of Pin1 R14A with PIN-221 and NMP bound in the
active site.

| | | | | | | | | | | | |
|---|---|---|---|---|---|---|---|---|---|---|---|
| ATOM | 1 | CB | LEU | | 7 | −56.410 | 14.231 | −69.954 | 1.00 | 39.38 | C |
| ATOM | 2 | CG | LEU | | 7 | −57.344 | 15.296 | −69.371 | 1.00 | 39.32 | C |
| ATOM | 3 | CD1 | LEU | | 7 | −56.606 | 16.076 | −68.301 | 1.00 | 38.07 | C |
| ATOM | 4 | CD2 | LEU | | 7 | −57.834 | 16.228 | −70.468 | 1.00 | 39.34 | C |
| ATOM | 5 | C | LEU | | 7 | −55.822 | 12.337 | −71.456 | 1.00 | 40.26 | C |
| ATOM | 6 | O | LEU | | 7 | −55.423 | 11.355 | −70.836 | 1.00 | 40.23 | O |
| ATOM | 7 | N | LEU | | 7 | −57.975 | 12.317 | −70.220 | 1.00 | 42.02 | N |
| ATOM | 8 | CA | LEU | | 7 | −56.971 | 13.180 | −70.919 | 1.00 | 40.26 | C |
| ATOM | 9 | N | PRO | | 8 | −55.268 | 12.716 | −72.619 | 1.00 | 40.49 | N |
| ATOM | 10 | CD | PRO | | 8 | −55.710 | 13.815 | −73.495 | 1.00 | 40.52 | C |
| ATOM | 11 | CA | PRO | | 8 | −54.150 | 11.982 | −73.230 | 1.00 | 39.38 | C |
| ATOM | 12 | CB | PRO | | 8 | −53.855 | 12.788 | −74.492 | 1.00 | 40.28 | C |
| ATOM | 13 | CG | PRO | | 8 | −55.190 | 13.374 | −74.845 | 1.00 | 41.43 | C |
| ATOM | 14 | C | PRO | | 8 | −52.943 | 11.919 | −72.295 | 1.00 | 38.38 | C |
| ATOM | 15 | O | PRO | | 8 | −52.859 | 12.668 | −71.318 | 1.00 | 38.41 | O |
| ATOM | 16 | N | PRO | | 9 | −51.986 | 11.024 | −72.596 | 1.00 | 36.88 | N |
| ATOM | 17 | CD | PRO | | 9 | −52.023 | 10.076 | −73.721 | 1.00 | 36.33 | C |
| ATOM | 18 | CA | PRO | | 9 | −50.766 | 10.836 | −71.801 | 1.00 | 34.43 | C |
| ATOM | 19 | CB | PRO | | 9 | −49.951 | 9.869 | −72.656 | 1.00 | 36.23 | C |
| ATOM | 20 | CG | PRO | | 9 | −51.010 | 9.037 | −73.302 | 1.00 | 36.23 | C |
| ATOM | 21 | C | PRO | | 9 | −49.994 | 12.119 | −71.494 | 1.00 | 33.36 | C |
| ATOM | 22 | O | PRO | | 9 | −49.608 | 12.861 | −72.402 | 1.00 | 31.76 | O |
| ATOM | 23 | N | GLY | | 10 | −49.772 | 12.366 | −70.206 | 1.00 | 31.73 | N |
| ATOM | 24 | CA | GLY | | 10 | −49.027 | 13.537 | −69.785 | 1.00 | 30.55 | C |
| ATOM | 25 | C | GLY | | 10 | −49.856 | 14.765 | −69.450 | 1.00 | 28.68 | C |
| ATOM | 26 | O | GLY | | 10 | −49.375 | 15.660 | −68.763 | 1.00 | 27.84 | O |
| ATOM | 27 | N | TRP | | 11 | −51.098 | 14.809 | −69.923 | 1.00 | 30.03 | N |
| ATOM | 28 | CA | TRP | | 11 | −51.958 | 15.962 | −69.677 | 1.00 | 30.64 | C |
| ATOM | 29 | CB | TRP | | 11 | −53.045 | 16.057 | −70.755 | 1.00 | 28.59 | C |
| ATOM | 30 | CG | TRP | | 11 | −52.515 | 16.400 | −72.108 | 1.00 | 27.18 | C |
| ATOM | 31 | CD2 | TRP | | 11 | −52.221 | 17.714 | −72.611 | 1.00 | 26.42 | C |
| ATOM | 32 | CE2 | TRP | | 11 | −51.726 | 17.558 | −73.922 | 1.00 | 24.60 | C |
| ATOM | 33 | CE3 | TRP | | 11 | −52.317 | 19.004 | −72.075 | 1.00 | 25.20 | C |
| ATOM | 34 | CD1 | TRP | | 11 | −52.199 | 15.528 | −73.110 | 1.00 | 25.73 | C |
| ATOM | 35 | NE1 | TRP | | 11 | −51.727 | 16.213 | −74.203 | 1.00 | 24.39 | N |
| ATOM | 36 | CZ2 | TRP | | 11 | −51.341 | 18.641 | −74.715 | 1.00 | 23.48 | C |
| ATOM | 37 | CZ3 | TRP | | 11 | −51.934 | 20.084 | −72.864 | 1.00 | 23.45 | C |
| ATOM | 38 | CH2 | TRP | | 11 | −51.447 | 19.893 | −74.168 | 1.00 | 25.15 | C |
| ATOM | 39 | C | TRP | | 11 | −52.613 | 15.996 | −68.303 | 1.00 | 31.89 | C |
| ATOM | 40 | O | TRP | | 11 | −52.976 | 14.966 | −67.740 | 1.00 | 32.03 | O |
| ATOM | 41 | N | GLU | | 12 | −52.758 | 17.206 | −67.773 | 1.00 | 33.73 | N |
| ATOM | 42 | CA | GLU | | 12 | −53.368 | 17.423 | −66.468 | 1.00 | 35.67 | C |
| ATOM | 43 | CB | GLU | | 12 | −52.305 | 17.362 | −65.368 | 1.00 | 39.46 | C |
| ATOM | 44 | CG | GLU | | 12 | −51.210 | 18.404 | −65.528 | 1.00 | 45.91 | C |
| ATOM | 45 | CD | GLU | | 12 | −50.226 | 18.418 | −64.372 | 1.00 | 50.27 | C |
| ATOM | 46 | OE1 | GLU | | 12 | −49.594 | 17.370 | −64.109 | 1.00 | 52.31 | O |
| ATOM | 47 | OE2 | GLU | | 12 | −50.086 | 19.482 | −63.729 | 1.00 | 52.45 | O |
| ATOM | 48 | C | GLU | | 12 | −54.032 | 18.796 | −66.443 | 1.00 | 35.73 | C |
| ATOM | 49 | O | GLU | | 12 | −53.668 | 19.684 | −67.215 | 1.00 | 33.75 | O |
| ATOM | 50 | N | LYS | | 13 | −55.010 | 18.964 | −65.562 | 1.00 | 36.27 | N |
| ATOM | 51 | CA | LYS | | 13 | −55.703 | 20.237 | −65.434 | 1.00 | 37.13 | C |
| ATOM | 52 | CB | LYS | | 13 | −57.121 | 20.040 | −64.894 | 1.00 | 40.41 | C |
| ATOM | 53 | CG | LYS | | 13 | −58.152 | 19.674 | −65.940 | 1.00 | 43.54 | C |
| ATOM | 54 | CD | LYS | | 13 | −59.558 | 19.798 | −65.366 | 1.00 | 46.43 | C |
| ATOM | 55 | CE | LYS | | 13 | −60.617 | 19.700 | −66.457 | 1.00 | 48.80 | C |
| ATOM | 56 | NZ | LYS | | 13 | −61.987 | 19.970 | −65.925 | 1.00 | 50.98 | N |
| ATOM | 57 | C | LYS | | 13 | −54.941 | 21.147 | −64.488 | 1.00 | 36.52 | C |
| ATOM | 58 | O | LYS | | 13 | −54.283 | 20.685 | −63.558 | 1.00 | 36.58 | O |
| ATOM | 59 | N | ALA | | 14 | −55.034 | 22.447 | −64.736 | 1.00 | 34.79 | N |
| ATOM | 60 | CA | ALA | | 14 | −54.364 | 23.428 | −63.901 | 1.00 | 34.15 | C |
| ATOM | 61 | CB | ALA | | 14 | −53.020 | 23.801 | −64.503 | 1.00 | 32.48 | C |
| ATOM | 62 | C | ALA | | 14 | −55.256 | 24.657 | −63.785 | 1.00 | 35.37 | C |
| ATOM | 63 | O | ALA | | 14 | −56.268 | 24.775 | −64.483 | 1.00 | 31.55 | O |
| ATOM | 64 | N | MET | | 15 | −54.870 | 25.574 | −62.906 | 1.00 | 36.26 | N |
| ATOM | 65 | CA | MET | | 15 | −55.638 | 26.792 | −62.679 | 1.00 | 35.96 | C |
| ATOM | 66 | CB | MET | | 15 | −56.068 | 26.856 | −61.209 | 1.00 | 38.28 | C |
| ATOM | 67 | CG | MET | | 15 | −56.721 | 28.159 | −60.790 | 1.00 | 40.70 | C |
| ATOM | 68 | SD | MET | | 15 | −58.190 | 28.498 | −61.760 | 1.00 | 44.63 | S |
| ATOM | 69 | CE | MET | | 15 | −59.303 | 27.228 | −61.121 | 1.00 | 45.72 | C |
| ATOM | 70 | C | MET | | 15 | −54.816 | 28.020 | −63.030 | 1.00 | 35.18 | C |
| ATOM | 71 | O | MET | | 15 | −53.701 | 28.190 | −62.542 | 1.00 | 33.75 | O |
| ATOM | 72 | N | SER | | 16 | −55.366 | 28.876 | −63.884 | 1.00 | 33.62 | N |
| ATOM | 73 | CA | SER | | 16 | −54.669 | 30.092 | −64.274 | 1.00 | 33.78 | C |
| ATOM | 74 | CB | SER | | 16 | −55.420 | 30.796 | −65.400 | 1.00 | 33.10 | C |
| ATOM | 75 | OG | SER | | 16 | −54.828 | 32.054 | −65.682 | 1.00 | 36.20 | O |
| ATOM | 76 | C | SER | | 16 | −54.568 | 31.028 | −63.077 | 1.00 | 33.89 | C |
| ATOM | 77 | O | SER | | 16 | −55.555 | 31.246 | −62.379 | 1.00 | 33.19 | O |
| ATOM | 78 | N | ARG | | 17 | −53.383 | 31.578 | −62.840 | 1.00 | 34.25 | N |
| ATOM | 79 | CA | ARG | | 17 | −53.192 | 32.498 | −61.720 | 1.00 | 36.71 | C |
| ATOM | 80 | CB | ARG | | 17 | −51.716 | 32.557 | −61.316 | 1.00 | 38.13 | C |
| ATOM | 81 | CG | ARG | | 17 | −51.320 | 31.506 | −60.300 | 1.00 | 41.18 | C |
| ATOM | 82 | CD | ARG | | 17 | −49.832 | 31.540 | −59.990 | 1.00 | 46.28 | C |
| ATOM | 83 | NE | ARG | | 17 | −49.467 | 30.534 | −58.995 | 1.00 | 50.33 | N |
| ATOM | 84 | CZ | ARG | | 17 | −49.796 | 29.246 | −59.075 | 1.00 | 52.00 | C |
| ATOM | 85 | NH1 | APG | | 17 | −50.499 | 28.790 | −60.105 | 1.00 | 54.92 | N |
| ATOM | 86 | NH2 | ARG | | 17 | −49.435 | 28.407 | −58.117 | 1.00 | 52.99 | N |
| ATOM | 87 | C | ARG | | 17 | −53.677 | 33.896 | −62.071 | 1.00 | 36.83 | C |
| ATOM | 88 | O | ARG | | 17 | −53.978 | 34.701 | −61.191 | 1.00 | 35.75 | O |
| ATOM | 89 | N | SER | | 18 | −53.752 | 34.178 | −63.366 | 1.00 | 36.51 | N |
| ATOM | 90 | CA | SER | | 18 | −54.187 | 35.478 | −63.844 | 1.00 | 38.08 | C |
| ATOM | 91 | CB | SER | | 18 | −53.535 | 35.780 | −65.198 | 1.00 | 39.30 | C |
| ATOM | 92 | OG | SER | | 18 | −52.124 | 35.815 | −65.091 | 1.00 | 43.42 | O |
| ATOM | 93 | C | SER | | 18 | −55.700 | 35.596 | −63.997 | 1.00 | 37.49 | C |
| ATOM | 94 | O | SER | | 18 | −56.317 | 36.518 | −63.463 | 1.00 | 38.43 | O |
| ATOM | 95 | N | SER | | 19 | −56.295 | 34.650 | −64.721 | 1.00 | 36.40 | N |
| ATOM | 96 | CA | SER | | 19 | −57.726 | 34.679 | −64.991 | 1.00 | 36.04 | C |
| ATOM | 97 | CB | SER | | 19 | −57.975 | 34.343 | −66.465 | 1.00 | 37.15 | C |
| ATOM | 98 | OG | SER | | 19 | −57.576 | 33.016 | −66.755 | 1.00 | 37.49 | O |
| ATOM | 99 | C | SER | | 19 | −58.601 | 33.779 | −64.139 | 1.00 | 35.08 | C |
| ATOM | 100 | O | SER | | 19 | −59.812 | 33.987 | −64.064 | 1.00 | 34.93 | O |
| ATOM | 101 | N | GLY | | 20 | −58.006 | 32.775 | −63.506 | 1.00 | 34.21 | N |
| ATOM | 102 | CA | GLY | | 20 | −58.797 | 31.874 | −62.689 | 1.00 | 32.88 | C |
| ATOM | 103 | C | GLY | | 20 | −59.521 | 30.849 | −63.546 | 1.00 | 32.25 | C |
| ATOM | 104 | O | GLY | | 20 | −60.344 | 30.075 | −63.056 | 1.00 | 33.75 | O |
| ATOM | 105 | N | ARG | | 21 | −59.221 | 30.851 | −64.841 | 1.00 | 30.49 | N |
| ATOM | 106 | CA | ARG | | 21 | −59.829 | 29.907 | −65.766 | 1.00 | 29.60 | C |
| ATOM | 107 | CB | ARG | | 21 | −59.903 | 30.512 | −67.169 | 1.00 | 32.07 | C |
| ATOM | 108 | CG | ARG | | 21 | −61.234 | 30.302 | −67.884 | 1.00 | 33.13 | C |
| ATOM | 109 | CD | ARG | | 21 | −61.118 | 30.676 | −69.355 | 1.00 | 34.83 | C |
| ATOM | 110 | NE | ARG | | 21 | −60.312 | 31.882 | −69.531 | 1.00 | 35.55 | N |
| ATOM | 111 | CZ | ARG | | 21 | −59.447 | 32.065 | −70.523 | 1.00 | 35.22 | C |
| ATOM | 112 | NH1 | ARG | | 21 | −59.269 | 31.125 | −71.446 | 1.00 | 33.97 | N |
| ATOM | 113 | NH2 | ARG | | 21 | −58.741 | 33.185 | −70.583 | 1.00 | 36.74 | N |
| ATOM | 114 | C | ARG | | 21 | −58.934 | 28.669 | −65.795 | 1.00 | 28.66 | C |
| ATOM | 115 | O | ARG | | 21 | −57.709 | 28.780 | −65.690 | 1.00 | 28.10 | O |
| ATOM | 116 | N | VAL | | 22 | −59.532 | 27.494 | −65.930 | 1.00 | 27.34 | N |
| ATOM | 117 | CA | VAL | | 22 | −58.734 | 26.275 | −65.972 | 1.00 | 27.98 | C |
| ATOM | 118 | CB | VAL | | 22 | −59.600 | 25.010 | −65.735 | 1.00 | 29.51 | C |
| ATOM | 119 | CG1 | VAL | | 22 | −60.324 | 25.114 | −64.400 | 1.00 | 32.84 | C |
| ATOM | 120 | CG2 | VAL | | 22 | −60.596 | 24.835 | −66.873 | 1.00 | 31.19 | C |
| ATOM | 121 | C | VAL | | 22 | −58.058 | 26.140 | −67.333 | 1.00 | 25.86 | C |
| ATOM | 122 | O | VAL | | 22 | −58.557 | 26.639 | −68.343 | 1.00 | 26.23 | O |
| ATOM | 123 | N | TYR | | 23 | −56.907 | 25.478 | −67.351 | 1.00 | 24.20 | N |
| ATOM | 124 | CA | TYR | | 23 | −56.191 | 25.244 | −68.594 | 1.00 | 23.75 | C |
| ATOM | 125 | CB | TYR | | 23 | −55.139 | 26.329 | −68.843 | 1.00 | 23.76 | C |
| ATOM | 126 | CG | TYR | | 23 | −53.951 | 26.323 | −67.904 | 1.00 | 26.59 | C |
| ATOM | 127 | CD1 | TYR | | 23 | −52.753 | 25.707 | −68.266 | 1.00 | 26.87 | C |
| ATOM | 128 | CE1 | TYR | | 23 | −51.647 | 25.739 | −67.422 | 1.00 | 28.14 | C |
| ATOM | 129 | CD2 | TYR | | 23 | −54.014 | 26.967 | −66.672 | 1.00 | 27.45 | C |
| ATOM | 130 | CE2 | TYR | | 23 | −52.924 | 27.002 | −65.824 | 1.00 | 28.77 | C |
| ATOM | 131 | CZ | TYR | | 23 | −51.739 | 26.389 | −66.206 | 1.00 | 30.47 | C |
| ATOM | 132 | OH | TYR | | 23 | −50.649 | 26.432 | −65.366 | 1.00 | 29.58 | O |
| ATOM | 133 | C | TYR | | 23 | −55.549 | 23.870 | −68.490 | 1.00 | 23.93 | C |
| ATOM | 134 | O | TYR | | 23 | −55.659 | 23.206 | −67.456 | 1.00 | 23.38 | O |
| ATOM | 135 | N | TYR | | 24 | −54.894 | 23.436 | −69.556 | 1.00 | 21.84 | N |
| ATOM | 136 | CA | TYR | | 24 | −54.268 | 22.123 | −69.561 | 1.00 | 22.71 | C |
| ATOM | 137 | CB | TYR | | 24 | −54.905 | 21.246 | −70.637 | 1.00 | 23.34 | C |
| ATOM | 138 | CG | TYR | | 24 | −56.400 | 21.111 | −70.450 | 1.00 | 27.59 | C |
| ATOM | 139 | CD1 | TYR | | 24 | −57.267 | 22.131 | −70.844 | 1.00 | 27.81 | C |
| ATOM | 140 | CE1 | TYR | | 24 | −58.633 | 22.044 | −70.608 | 1.00 | 29.99 | C |
| ATOM | 141 | CD2 | TYR | | 24 | −56.939 | 19.996 | −69.816 | 1.00 | 28.78 | C |
| ATOM | 142 | CE2 | TYR | | 24 | −58.308 | 19.897 | −69.573 | 1.00 | 31.48 | C |
| ATOM | 143 | CZ | TYR | | 24 | −59.146 | 20.924 | −69.968 | 1.00 | 32.75 | C |
| ATOM | 144 | OH | TYR | | 24 | −60.498 | 20.833 | −69.714 | 1.00 | 34.12 | O |
| ATOM | 145 | C | TYR | | 24 | −52.773 | 22.233 | −69.770 | 1.00 | 21.33 | C |
| ATOM | 146 | O | TYR | | 24 | −52.299 | 23.081 | −70.526 | 1.00 | 19.93 | O |
| ATOM | 147 | N | PHE | | 25 | −52.042 | 21.364 | −69.085 | 1.00 | 22.74 | N |
| ATOM | 148 | CA | PHE | | 25 | −50.588 | 21.335 | −69.136 | 1.00 | 23.11 | C |
| ATOM | 149 | CB | PHE | | 25 | −50.039 | 21.901 | −67.823 | 1.00 | 25.42 | C |
| ATOM | 150 | CG | PHE | | 25 | −48.561 | 21.717 | −67.649 | 1.00 | 28.35 | C |
| ATOM | 151 | CD1 | PHE | | 25 | −47.657 | 22.489 | −68.377 | 1.00 | 29.07 | C |
| ATOM | 152 | CD2 | PHE | | 25 | −48.069 | 20.760 | −66.762 | 1.00 | 30.03 | C |
| ATOM | 153 | CE1 | PHE | | 25 | −46.273 | 22.307 | −68.228 | 1.00 | 29.42 | C |
| ATOM | 154 | CE2 | PHE | | 25 | −46.694 | 20.570 | −66.607 | 1.00 | 30.76 | C |
| ATOM | 155 | CZ | PHE | | 25 | −45.794 | 21.349 | −67.344 | 1.00 | 30.48 | C |
| ATOM | 156 | C | PHE | | 25 | −50.149 | 19.888 | −69.307 | 1.00 | 23.65 | C |
| ATOM | 157 | O | PHE | | 25 | −50.804 | 18.972 | −68.804 | 1.00 | 23.29 | O |
| ATOM | 158 | N | ASN | | 26 | −49.052 | 19.682 | −70.033 | 1.00 | 22.66 | N |
| ATOM | 159 | CA | ASN | | 26 | −48.518 | 18.339 | −70.251 | 1.00 | 22.04 | C |
| ATOM | 160 | CB | ASN | | 26 | −48.417 | 18.039 | −71.747 | 1.00 | 20.72 | C |
| ATOM | 161 | CG | ASN | | 26 | −48.091 | 16.581 | −72.021 | 1.00 | 21.83 | C |
| ATOM | 162 | OD1 | ASN | | 26 | −47.036 | 16.086 | −71.617 | 1.00 | 20.44 | O |
| ATOM | 163 | ND2 | ASN | | 26 | −48.994 | 15.886 | −72.706 | 1.00 | 20.94 | N |
| ATOM | 164 | C | ASN | | 26 | −47.137 | 18.308 | −69.604 | 1.00 | 20.26 | C |
| ATOM | 165 | O | ASN | | 26 | −46.241 | 19.042 | −70.018 | 1.00 | 19.47 | O |
| ATOM | 166 | N | HIS | | 27 | −46.968 | 17.468 | −68.583 | 1.00 | 21.74 | N |
| ATOM | 167 | CA | HIS | | 27 | −45.694 | 17.411 | −67.875 | 1.00 | 22.19 | C |
| ATOM | 168 | CB | HIS | | 27 | −45.887 | 16.871 | −66.445 | 1.00 | 23.05 | C |
| ATOM | 169 | CG | HIS | | 27 | −46.465 | 15.492 | −66.380 | 1.00 | 25.37 | C |
| ATOM | 170 | CD2 | HIS | | 27 | −47.714 | 15.062 | −66.077 | 1.00 | 26.79 | C |
| ATOM | 171 | ND1 | HIS | | 27 | −45.724 | 14.359 | −66.643 | 1.00 | 29.55 | N |
| ATOM | 172 | CE1 | HIS | | 27 | −46.491 | 13.291 | −66.503 | 1.00 | 29.10 | C |
| ATOM | 173 | NE2 | HIS | | 27 | −47.703 | 13.692 | −66.160 | 1.00 | 28.90 | N |
| ATOM | 174 | C | HIS | | 27 | −44.590 | 16.643 | −68.590 | 1.00 | 22.17 | C |
| ATOM | 175 | O | HIS | | 27 | −43.476 | 16.549 | −68.086 | 1.00 | 21.17 | O |
| ATOM | 176 | N | ILE | | 28 | −44.893 | 16.112 | −69.768 | 1.00 | 22.93 | N |
| ATOM | 177 | CA | ILE | | 28 | −43.894 | 15.391 | −70.546 | 1.00 | 23.87 | C |
| ATOM | 178 | CB | ILE | | 28 | −44.479 | 14.114 | −71.199 | 1.00 | 23.89 | C |
| ATOM | 179 | CG2 | ILE | | 28 | −43.361 | 13.330 | −71.892 | 1.00 | 23.45 | C |
| ATOM | 180 | CG1 | ILE | | 28 | −45.128 | 13.228 | −70.135 | 1.00 | 24.05 | C |
| ATOM | 181 | CD1 | ILE | | 28 | −45.862 | 12.019 | −70.704 | 1.00 | 23.04 | C |
| ATOM | 182 | C | ILE | | 28 | −43.372 | 16.302 | −71.665 | 1.00 | 24.77 | C |
| ATOM | 183 | O | ILE | | 28 | −42.169 | 16.355 | −71.926 | 1.00 | 25.85 | O |
| ATOM | 184 | N | THR | | 29 | −44.281 | 17.028 | −72.311 | 1.00 | 23.94 | N |
| ATOM | 185 | CA | THR | | 29 | −43.916 | 17.923 | −73.417 | 1.00 | 22.06 | C |
| ATOM | 186 | CB | THR | | 29 | −44.960 | 17.876 | −74.528 | 1.00 | 21.36 | C |
| ATOM | 187 | OG1 | THR | | 29 | −46.156 | 18.514 | −74.067 | 1.00 | 20.81 | O |
| ATOM | 188 | CG2 | THR | | 29 | −45.280 | 16.446 | −74.906 | 1.00 | 19.80 | C |
| ATOM | 189 | C | THR | | 29 | −43.815 | 19.382 | −72.990 | 1.00 | 21.58 | C |
| ATOM | 190 | O | THR | | 29 | −43.270 | 20.209 | −73.721 | 1.00 | 20.87 | O |
| ATOM | 191 | N | ASN | | 30 | −44.344 | 19.685 | −71.808 | 1.00 | 22.03 | N |
| ATOM | 192 | CA | ASN | | 30 | −44.376 | 21.043 | −71.266 | 1.00 | 20.90 | C |
| ATOM | 193 | CB | ASN | | 30 | −42.970 | 21.631 | −71.141 | 1.00 | 20.66 | C |
| ATOM | 194 | CG | ASN | | 30 | −42.218 | 21.078 | −69.941 | 1.00 | 23.68 | C |
| ATOM | 195 | OD1 | ASN | | 30 | −42.825 | 20.760 | −68.920 | 1.00 | 22.19 | O |
| ATOM | 196 | ND2 | ASN | | 30 | −40.894 | 20.974 | −70.056 | 1.00 | 21.52 | N |
| ATOM | 197 | C | ASN | | 30 | −45.276 | 21.987 | −72.071 | 1.00 | 19.91 | C |
| ATOM | 198 | O | ASN | | 30 | −45.145 | 23.209 | −71.990 | 1.00 | 20.71 | O |
| ATOM | 199 | N | ALA | | 31 | −46.181 | 21.413 | −72.854 | 1.00 | 19.06 | N |
| ATOM | 200 | CA | ALA | | 31 | −47.122 | 22.214 | −73.625 | 1.00 | 20.25 | C |
| ATOM | 201 | CB | ALA | | 31 | −47.764 | 21.371 | −74.707 | 1.00 | 21.52 | C |
| ATOM | 202 | C | ALA | | 31 | −48.192 | 22.684 | −72.647 | 1.00 | 21.26 | C |
| ATOM | 203 | O | ALA | | 31 | −48.469 | 22.011 | −71.644 | 1.00 | 20.03 | O |
| ATOM | 204 | N | SER | | 32 | −48.782 | 23.844 | −72.923 | 1.00 | 21.11 | N |
| ATOM | 205 | CA | SER | | 32 | −49.850 | 24.381 | −72.083 | 1.00 | 21.25 | C |
| ATOM | 206 | CB | SER | | 32 | −49.290 | 25.313 | −70.997 | 1.00 | 22.58 | C |
| ATOM | 207 | OG | SER | | 32 | −48.492 | 26.346 | −71.541 | 1.00 | 25.45 | O |
| ATOM | 208 | C | SER | | 32 | −50.811 | 25.120 | −73.004 | 1.00 | 21.97 | C |
| ATOM | 209 | O | SER | | 32 | −50.387 | 25.853 | −73.893 | 1.00 | 20.51 | O |
| ATOM | 210 | N | GLN | | 33 | −52.106 | 24.903 | −72.800 | 1.00 | 21.88 | N |
| ATOM | 211 | CA | GLN | | 33 | −53.128 | 25.507 | −73.651 | 1.00 | 22.81 | C |
| ATOM | 212 | CB | GLN | | 33 | −53.332 | 24.615 | −74.881 | 1.00 | 22.40 | C |
| ATOM | 213 | CG | GLN | | 33 | −53.830 | 23.203 | −74.539 | 1.00 | 24.37 | C |
| ATOM | 214 | CD | GLN | | 33 | −53.673 | 22.217 | −75.686 | 1.00 | 27.62 | C |
| ATOM | 215 | OE1 | GLN | | 33 | −54.470 | 21.280 | −75.838 | 1.00 | 29.68 | O |
| ATOM | 216 | NE2 | GLN | | 33 | −52.635 | 22.407 | −76.491 | 1.00 | 23.21 | N |
| ATOM | 217 | C | GLN | | 33 | −54.447 | 25.647 | −72.894 | 1.00 | 21.82 | C |
| ATOM | 218 | O | GLN | | 33 | −54.637 | 25.009 | −71.861 | 1.00 | 23.05 | O |
| ATOM | 219 | N | TRP | | 34 | −55.353 | 26.476 | −73.408 | 1.00 | 22.50 | N |
| ATOM | 220 | CA | TRP | | 34 | −56.663 | 26.659 | −72.771 | 1.00 | 22.75 | C |
| ATOM | 221 | CB | TRP | | 34 | −57.331 | 27.968 | −73.226 | 1.00 | 21.17 | C |
| ATOM | 222 | CG | TRP | | 34 | −56.623 | 29.216 | −72.792 | 1.00 | 18.63 | C |
| ATOM | 223 | CD2 | TRP | | 34 | −56.430 | 29.669 | −71.446 | 1.00 | 19.23 | C |
| ATOM | 224 | CE2 | TRP | | 34 | −55.679 | 30.861 | −71.510 | 1.00 | 20.74 | C |
| ATOM | 225 | CE3 | TRP | | 34 | −56.816 | 29.180 | −70.191 | 1.00 | 18.54 | C |
| ATOM | 226 | CD1 | TRP | | 34 | −56.009 | 30.129 | −73.596 | 1.00 | 21.25 | C |
| ATOM | 227 | NE1 | TRP | | 34 | −55.438 | 31.121 | −72.835 | 1.00 | 22.11 | N |
| ATOM | 228 | CZ2 | TRP | | 34 | −55.307 | 31.573 | −70.372 | 1.00 | 21.10 | C |
| ATOM | 229 | CZ3 | TRP | | 34 | −56.444 | 29.886 | −69.061 | 1.00 | 21.07 | C |
| ATOM | 230 | CH2 | TRP | | 34 | −55.694 | 31.072 | −69.159 | 1.00 | 21.99 | C |
| ATOM | 231 | C | TRP | | 34 | −57.585 | 25.511 | −73.159 | 1.00 | 24.08 | C |
| ATOM | 232 | O | TRP | | 34 | −58.355 | 25.004 | −72.340 | 1.00 | 24.51 | O |
| ATOM | 233 | N | GLU | | 35 | −57.496 | 25.119 | −74.424 | 1.00 | 25.70 | N |
| ATOM | 234 | CA | GLU | | 35 | −58.322 | 24.064 | −74.998 | 1.00 | 27.30 | C |
| ATOM | 235 | CB | GLU | | 35 | −58.157 | 24.057 | −76.522 | 1.00 | 28.78 | C |
| ATOM | 236 | CG | GLU | | 35 | −58.531 | 25.365 | −77.218 | 1.00 | 28.73 | C |
| ATOM | 237 | CD | GLU | | 35 | −57.436 | 26.415 | −77.145 | 1.00 | 29.39 | C |
| ATOM | 238 | OE1 | GLU | | 35 | −57.697 | 27.578 | −77.528 | 1.00 | 29.15 | O |
| ATOM | 239 | OE2 | GLU | | 35 | −56.311 | 26.080 | −76.716 | 1.00 | 26.63 | O |
| ATOM | 240 | C | GLU | | 35 | −58.045 | 22.662 | −74.460 | 1.00 | 30.11 | C |
| ATOM | 241 | O | GLU | | 35 | −56.893 | 22.274 | −74.269 | 1.00 | 28.19 | O |
| ATOM | 242 | N | ARG | | 36 | −59.112 | 21.902 | −74.226 | 1.00 | 31.77 | N |
| ATOM | 243 | CA | ARG | | 36 | −58.977 | 20.541 | −73.731 | 1.00 | 36.40 | C |
| ATOM | 244 | CB | ARG | | 36 | −60.354 | 19.966 | −73.373 | 1.00 | 39.28 | C |
| ATOM | 245 | CG | ARG | | 36 | −60.309 | 18.625 | −72.647 | 1.00 | 44.26 | C |
| ATOM | 246 | CD | ARG | | 36 | −61.710 | 18.051 | −72.475 | 1.00 | 49.03 | C |
| ATOM | 247 | NE | ARG | | 36 | −61.726 | 16.780 | −71.750 | 1.00 | 52.82 | N |
| ATOM | 248 | CZ | ARG | | 36 | −61.634 | 16.661 | −70.426 | 1.00 | 54.57 | C |
| ATOM | 249 | NH1 | ARG | | 36 | −61.522 | 17.741 | −69.662 | 1.00 | 55.16 | N |
| ATOM | 250 | NH2 | ARG | | 36 | −61.665 | 15.459 | −69.865 | 1.00 | 55.50 | N |
| ATOM | 251 | C | ARG | | 36 | −58.344 | 19.709 | −74.848 | 1.00 | 37.21 | C |
| ATOM | 252 | O | ARG | | 36 | −58.843 | 19.686 | −75.973 | 1.00 | 37.46 | O |
| ATOM | 253 | N | PRO | | 37 | −57.230 | 19.020 | −74.553 | 1.00 | 37.60 | N |
| ATOM | 254 | CD | PRO | | 37 | −56.580 | 18.870 | −73.241 | 1.00 | 36.82 | C |
| ATOM | 255 | CA | PRO | | 37 | −56.556 | 18.203 | −75.570 | 1.00 | 39.97 | C |
| ATOM | 256 | CB | PRO | | 37 | −55.318 | 17.692 | −74.832 | 1.00 | 38.84 | C |
| ATOM | 257 | CG | PRO | | 37 | −55.794 | 17.590 | −73.419 | 1.00 | 37.32 | C |
| ATOM | 258 | C | PRO | | 37 | −57.415 | 17.066 | −76.137 | 1.00 | 42.18 | C |
| ATOM | 259 | O | PRO | | 37 | −58.161 | 16.409 | −75.406 | 1.00 | 42.61 | O |
| ATOM | 260 | N | SER | | 38 | −57.293 | 16.848 | −77.444 | 1.00 | 44.85 | N |
| ATOM | 261 | CA | SER | | 38 | −58.037 | 15.802 | −78.146 | 1.00 | 47.39 | C |
| ATOM | 262 | CB | SER | | 38 | −57.602 | 15.745 | −79.614 | 1.00 | 48.61 | C |
| ATOM | 263 | OG | SER | | 38 | −57.746 | 17.005 | −80.248 | 1.00 | 49.94 | O |
| ATOM | 264 | C | SER | | 38 | −57.814 | 14.436 | −77.506 | 1.00 | 47.97 | C |
| ATOM | 265 | O | SER | | 38 | −56.707 | 13.900 | −77.542 | 1.00 | 48.17 | O |
| ATOM | 266 | N | GLU | | 51 | −48.869 | 3.556 | −78.910 | 1.00 | 56.73 | N |
| ATOM | 267 | CA | GLU | | 51 | −48.210 | 4.726 | −79.479 | 1.00 | 56.61 | C |
| ATOM | 268 | CB | GLU | | 51 | −47.139 | 5.253 | −78.516 | 1.00 | 56.78 | C |
| ATOM | 269 | CG | GLU | | 51 | −45.888 | 4.388 | −78.449 | 1.00 | 56.13 | C |
| ATOM | 270 | CD | GLU | | 51 | −44.790 | 4.995 | −77.592 | 1.00 | 56.29 | C |
| ATOM | 271 | OE1 | GLU | | 51 | −43.677 | 4.431 | −77.577 | 1.00 | 56.48 | O |
| ATOM | 272 | OE2 | GLU | | 51 | −45.038 | 6.028 | −76.931 | 1.00 | 56.26 | O |
| ATOM | 273 | C | GLU | | 51 | −47.554 | 4.362 | −80.808 | 1.00 | 56.01 | C |
| ATOM | 274 | O | GLU | | 51 | −47.310 | 3.186 | −81.087 | 1.00 | 56.46 | O |
| ATOM | 275 | N | PRO | | 52 | −47.263 | 5.371 | −81.650 | 1.00 | 54.73 | N |
| ATOM | 276 | CD | PRO | | 52 | −47.629 | 6.790 | −81.493 | 1.00 | 54.54 | C |
| ATOM | 277 | CA | PRO | | 52 | −46.631 | 5.142 | −82.954 | 1.00 | 52.81 | C |
| ATOM | 278 | CB | PRO | | 52 | −46.516 | 6.550 | −83.533 | 1.00 | 53.21 | C |
| ATOM | 279 | CG | PRO | | 52 | −47.684 | 7.260 | −82.926 | 1.00 | 54.26 | C |
| ATOM | 280 | C | PRO | | 52 | −45.264 | 4.476 | −82.805 | 1.00 | 51.22 | C |
| ATOM | 281 | O | PRO | | 52 | −44.590 | 4.649 | −81.790 | 1.00 | 51.92 | O |
| ATOM | 282 | N | ALA | | 53 | −44.860 | 3.717 | −83.818 | 1.00 | 48.36 | N |
| ATOM | 283 | CA | ALA | | 53 | −43.569 | 3.044 | −83.795 | 1.00 | 45.29 | C |
| ATOM | 284 | CB | ALA | | 53 | −43.554 | 1.913 | −84.807 | 1.00 | 45.58 | C |
| ATOM | 285 | C | ALA | | 53 | −42.482 | 4.054 | −84.128 | 1.00 | 43.82 | C |
| ATOM | 286 | O | ALA | | 53 | −41.333 | 3.927 | −83.696 | 1.00 | 41.97 | O |
| ATOM | 287 | N | ARG | | 54 | −42.862 | 5.064 | −84.906 | 1.00 | 41.76 | N |
| ATOM | 288 | CA | ARG | | 54 | −41.940 | 6.116 | −85.315 | 1.00 | 39.39 | C |
| ATOM | 289 | CB | ARG | | 54 | −41.325 | 5.779 | −86.675 | 1.00 | 41.84 | C |
| ATOM | 290 | CG | ARG | | 54 | −40.620 | 4.434 | −86.734 | 1.00 | 46.43 | C |
| ATOM | 291 | CD | ARG | | 54 | −40.372 | 4.011 | −88.173 | 1.00 | 48.42 | C |
| ATOM | 292 | NE | ARG | | 54 | −39.518 | 4.952 | −88.891 | 1.00 | 50.78 | N |
| ATOM | 293 | CZ | ARG | | 54 | −38.237 | 5.169 | −88.602 | 1.00 | 52.53 | C |
| ATOM | 294 | NH1 | ARG | | 54 | −37.660 | 4.513 | −87.603 | 1.00 | 54.12 | N |
| ATOM | 295 | NH2 | ARG | | 54 | −37.533 | 6.034 | −89.320 | 1.00 | 53.96 | N |
| ATOM | 296 | C | ARG | | 54 | −42.676 | 7.441 | −85.429 | 1.00 | 36.18 | C |
| ATOM | 297 | O | ARG | | 54 | −43.873 | 7.479 | −85.710 | 1.00 | 34.65 | O |
| ATOM | 298 | N | VAL | | 55 | −41.950 | 8.527 | −85.203 | 1.00 | 32.83 | N |
| ATOM | 299 | CA | VAL | | 55 | −42.524 | 9.857 | −85.326 | 1.00 | 30.15 | C |
| ATOM | 300 | CB | VAL | | 55 | −42.811 | 10.517 | −83.941 | 1.00 | 28.86 | C |
| ATOM | 301 | CG1 | VAL | | 55 | −43.813 | 9.682 | −83.161 | 1.00 | 28.16 | C |
| ATOM | 302 | CG2 | VAL | | 55 | −41.515 | 10.690 | −83.153 | 1.00 | 27.14 | C |
| ATOM | 303 | C | VAL | | 55 | −41.524 | 10.725 | −86.067 | 1.00 | 28.50 | C |
| ATOM | 304 | O | VAL | | 55 | −40.322 | 10.451 | −86.068 | 1.00 | 28.45 | O |
| ATOM | 305 | N | ARG | | 56 | −42.026 | 11.759 | −86.724 | 1.00 | 25.58 | N |
| ATOM | 306 | CA | ARG | | 56 | −41.154 | 12.678 | −87.422 | 1.00 | 22.50 | C |
| ATOM | 307 | CB | ARG | | 56 | −41.541 | 12.780 | −88.894 | 1.00 | 21.90 | C |
| ATOM | 308 | CG | ARG | | 56 | −40.607 | 13.673 | −89.679 | 1.00 | 22.73 | C |
| ATOM | 309 | CD | ARG | | 56 | −41.005 | 13.740 | −91.136 | 1.00 | 22.20 | C |
| ATOM | 310 | NE | ARG | | 56 | −40.028 | 14.491 | −91.919 | 1.00 | 25.01 | N |
| ATOM | 311 | CZ | ARG | | 56 | −40.148 | 14.723 | −93.220 | 1.00 | 26.61 | C |
| ATOM | 312 | NH1 | ARG | | 56 | −41.209 | 14.259 | −93.876 | 1.00 | 25.11 | N |
| ATOM | 313 | NH2 | ARG | | 56 | −39.217 | 15.417 | −93.862 | 1.00 | 24.53 | N |
| ATOM | 314 | C | ARG | | 56 | −41.325 | 14.026 | −86.739 | 1.00 | 22.03 | C |
| ATOM | 315 | O | ARG | | 56 | −42.455 | 14.486 | −86.541 | 1.00 | 21.05 | O |
| ATOM | 316 | N | CYS | | 57 | −40.207 | 14.652 | −86.365 | 1.00 | 19.68 | N |
| ATOM | 317 | CA | CYS | | 57 | −40.254 | 15.946 | −85.697 | 1.00 | 18.91 | C |
| ATOM | 318 | CB | CYS | | 57 | −40.013 | 15.790 | −84.185 | 1.00 | 17.90 | C |
| ATOM | 319 | SG | CYS | | 57 | −41.212 | 14.769 | −83.297 | 1.00 | 21.12 | S |
| ATOM | 320 | C | CYS | | 57 | −39.227 | 16.937 | −86.213 | 1.00 | 20.35 | C |
| ATOM | 321 | O | CYS | | 57 | −38.198 | 16.568 | −86.795 | 1.00 | 19.47 | O |
| ATOM | 322 | N | SER | | 58 | −39.533 | 18.208 | −85.998 | 1.00 | 18.42 | N |
| ATOM | 323 | CA | SER | | 58 | −38.624 | 19.283 | −86.331 | 1.00 | 18.34 | C |
| ATOM | 324 | CB | SER | | 58 | −39.271 | 20.285 | −87.284 | 1.00 | 18.37 | C |
| ATOM | 325 | OG | SER | | 58 | −39.552 | 19.688 | −88.538 | 1.00 | 21.68 | O |
| ATOM | 326 | C | SER | | 58 | −38.393 | 19.915 | −84.959 | 1.00 | 20.07 | C |
| ATOM | 327 | O | SER | | 58 | −39.166 | 19.683 | −84.022 | 1.00 | 20.92 | O |
| ATOM | 328 | N | HIS | | 59 | −37.321 | 20.680 | −84.811 | 1.00 | 21.09 | N |
| ATOM | 329 | CA | HIS | | 59 | −37.073 | 21.319 | −83.532 | 1.00 | 21.22 | C |
| ATOM | 330 | CB | HIS | | 59 | −36.325 | 20.381 | −82.574 | 1.00 | 22.72 | C |
| ATOM | 331 | CG | HIS | | 59 | −34.879 | 20.197 | −82.911 | 1.00 | 23.97 | C |
| ATOM | 332 | CD2 | HIS | | 59 | −34.267 | 19.865 | −84.073 | 1.00 | 24.66 | C |
| ATOM | 333 | ND1 | HIS | | 59 | −33.873 | 20.364 | −81.984 | 1.00 | 25.54 | N |
| ATOM | 334 | CE1 | HIS | | 59 | −32.704 | 20.145 | −82.561 | 1.00 | 26.41 | C |
| ATOM | 335 | NE2 | HIS | | 59 | −32.915 | 19.841 | −83.827 | 1.00 | 25.59 | N |
| ATOM | 336 | C | HIS | | 59 | −36.266 | 22.582 | −83.718 | 1.00 | 20.66 | C |
| ATOM | 337 | O | HIS | | 59 | −35.702 | 22.830 | −84.788 | 1.00 | 18.63 | O |
| ATOM | 338 | N | LEU | | 60 | −36.247 | 23.380 | −82.658 | 1.00 | 20.46 | N |
| ATOM | 339 | CA | LEU | | 60 | −35.506 | 24.627 | −82.613 | 1.00 | 20.41 | C |
| ATOM | 340 | CB | LEU | | 60 | −36.452 | 25.831 | −82.529 | 1.00 | 19.84 | C |
| ATOM | 341 | CG | LEU | | 60 | −35.803 | 27.203 | −82.759 | 1.00 | 20.17 | C |
| ATOM | 342 | CD1 | LEU | | 60 | −36.888 | 28.241 | −83.017 | 1.00 | 21.15 | C |
| ATOM | 343 | CD2 | LEU | | 60 | −34.950 | 27.594 | −81.562 | 1.00 | 17.61 | C |
| ATOM | 344 | C | LEU | | 60 | −34.722 | 24.484 | −81.321 | 1.00 | 22.65 | C |
| ATOM | 345 | O | LEU | | 60 | −35.301 | 24.363 | −80.238 | 1.00 | 22.36 | O |
| ATOM | 346 | N | LEU | | 61 | −33.400 | 24.475 | −81.435 | 1.00 | 22.98 | N |
| ATOM | 347 | CA | LEU | | 61 | −32.558 | 24.321 | −80.264 | 1.00 | 23.30 | C |
| ATOM | 348 | CB | LEU | | 61 | −31.530 | 23.205 | −80.509 | 1.00 | 24.05 | C |
| ATOM | 349 | CG | LEU | | 61 | −30.413 | 23.035 | −79.470 | 1.00 | 25.37 | C |
| ATOM | 350 | CD1 | LEU | | 61 | −30.999 | 22.653 | −78.122 | 1.00 | 23.91 | C |
| ATOM | 351 | CD2 | LEU | | 61 | −29.435 | 21.965 | −79.950 | 1.00 | 25.78 | C |
| ATOM | 352 | C | LEU | | 61 | −31.834 | 25.599 | −79.893 | 1.00 | 22.89 | C |
| ATOM | 353 | O | LEU | | 61 | −31.290 | 26.289 | −80.750 | 1.00 | 23.54 | O |
| ATOM | 354 | N | VAL | | 62 | −31.858 | 25.924 | −78.608 | 1.00 | 24.14 | N |
| ATOM | 355 | CA | VAL | | 62 | −31.151 | 27.088 | −78.112 | 1.00 | 24.92 | C |
| ATOM | 356 | CB | VAL | | 62 | −32.101 | 28.132 | −77.477 | 1.00 | 26.28 | C |
| ATOM | 357 | CG1 | VAL | | 62 | −31.295 | 29.293 | −76.906 | 1.00 | 25.58 | C |
| ATOM | 358 | CG2 | VAL | | 62 | −33.064 | 28.660 | −78.533 | 1.00 | 25.43 | C |
| ATOM | 359 | C | VAL | | 62 | −30.191 | 26.543 | −77.063 | 1.00 | 26.85 | C |
| ATOM | 360 | O | VAL | | 62 | −30.606 | 25.972 | −76.048 | 1.00 | 25.08 | O |
| ATOM | 361 | N | LYS | | 63 | −28.900 | 26.685 | −77.341 | 1.00 | 27.56 | N |
| ATOM | 362 | CA | LYS | | 63 | −27.870 | 26.209 | −76.433 | 1.00 | 30.11 | C |
| ATOM | 363 | CB | LYS | | 63 | −26.628 | 25.774 | −77.221 | 1.00 | 29.50 | C |
| ATOM | 364 | CG | LYS | | 63 | −26.833 | 24.509 | −78.031 | 1.00 | 32.93 | C |
| ATOM | 365 | CD | LYS | | 63 | −25.532 | 24.031 | −78.658 | 1.00 | 35.79 | C |
| ATOM | 366 | CE | LYS | | 63 | −25.624 | 22.564 | −79.022 | 1.00 | 36.57 | C |
| ATOM | 367 | NZ | LYS | | 63 | −24.357 | 22.064 | −79.607 | 1.00 | 39.73 | N |
| ATOM | 368 | C | LYS | | 63 | −27.494 | 27.300 | −75.451 | 1.00 | 29.74 | C |
| ATOM | 369 | O | LYS | | 63 | −27.847 | 28.467 | −75.642 | 1.00 | 29.85 | O |
| ATOM | 370 | N | HIS | | 64 | −26.789 | 26.909 | −74.396 | 1.00 | 30.90 | N |
| ATOM | 371 | CA | HIS | | 64 | −26.335 | 27.845 | −73.376 | 1.00 | 33.12 | C |
| ATOM | 372 | CB | HIS | | 64 | −27.344 | 27.912 | −72.222 | 1.00 | 32.01 | C |
| ATOM | 373 | CG | HIS | | 64 | −27.772 | 26.571 | −71.713 | 1.00 | 32.69 | C |
| ATOM | 374 | CD2 | HIS | | 64 | −28.925 | 25.883 | −71.880 | 1.00 | 32.39 | C |
| ATOM | 375 | ND1 | HIS | | 64 | −26.952 | 25.766 | −70.951 | 1.00 | 32.58 | N |
| ATOM | 376 | CE1 | HIS | | 64 | −27.582 | 24.637 | −70.674 | 1.00 | 33.98 | C |
| ATOM | 377 | NE2 | HIS | | 64 | −28.783 | 24.684 | −71.225 | 1.00 | 33.29 | N |
| ATOM | 378 | C | HIS | | 64 | −24.969 | 27.388 | −72.875 | 1.00 | 34.96 | C |
| ATOM | 379 | O | HIS | | 64 | −24.488 | 26.311 | −73.253 | 1.00 | 34.80 | O |
| ATOM | 380 | N | SER | | 65 | −24.347 | 28.203 | −72.028 | 1.00 | 35.87 | N |
| ATOM | 381 | CA | SER | | 65 | −23.023 | 27.890 | −71.493 | 1.00 | 37.77 | C |
| ATOM | 382 | CB | SER | | 65 | −22.551 | 29.014 | −70.563 | 1.00 | 37.31 | C |
| ATOM | 383 | OG | SER | | 65 | −23.420 | 29.172 | −69.458 | 1.00 | 38.71 | O |
| ATOM | 384 | C | SER | | 65 | −22.919 | 26.539 | −70.776 | 1.00 | 38.42 | C |
| ATOM | 385 | O | SER | | 65 | −21.818 | 26.040 | −70.554 | 1.00 | 40.45 | O |
| ATOM | 386 | N | GLN | | 66 | −24.050 | 25.940 | −70.414 | 1.00 | 38.44 | N |
| ATOM | 387 | CA | GLN | | 66 | −24.008 | 24.645 | −69.745 | 1.00 | 39.20 | C |
| ATOM | 388 | CB | GLN | | 66 | −24.954 | 24.607 | −68.539 | 1.00 | 42.80 | C |
| ATOM | 389 | CG | GLN | | 66 | −24.410 | 25.271 | −67.277 | 1.00 | 45.54 | C |
| ATOM | 390 | CD | GLN | | 66 | −25.172 | 24.848 | −66.020 | 1.00 | 48.36 | C |
| ATOM | 391 | OE1 | GLN | | 66 | −25.111 | 23.685 | −65.598 | 1.00 | 47.36 | O |
| ATOM | 392 | NE2 | GLN | | 66 | −25.898 | 25.790 | −65.423 | 1.00 | 48.10 | N |
| ATOM | 393 | C | GLN | | 66 | −24.340 | 23.480 | −70.672 | 1.00 | 38.29 | C |
| ATOM | 394 | O | GLN | | 66 | −24.460 | 22.343 | −70.223 | 1.00 | 38.60 | O |
| ATOM | 395 | N | SER | | 67 | −24.501 | 23.760 | −71.963 | 1.00 | 37.66 | N |
| ATOM | 396 | CA | SER | | 67 | −24.798 | 22.708 | −72.928 | 1.00 | 37.44 | C |
| ATOM | 397 | CB | SER | | 67 | −25.128 | 23.319 | −74.295 | 1.00 | 37.14 | C |
| ATOM | 398 | OG | SER | | 67 | −26.273 | 24.158 | −74.224 | 1.00 | 34.86 | O |
| ATOM | 399 | C | SER | | 67 | −23.556 | 21.829 | −73.035 | 1.00 | 38.94 | C |
| ATOM | 400 | O | SER | | 67 | −22.436 | 22.337 | −73.010 | 1.00 | 37.92 | O |
| ATOM | 401 | N | ARG | | 68 | −23.748 | 20.517 | −73.145 | 1.00 | 40.00 | N |
| ATOM | 402 | CA | ARG | | 68 | −22.615 | 19.598 | −73.237 | 1.00 | 43.53 | C |
| ATOM | 403 | CB | ARG | | 68 | −23.069 | 18.228 | −73.742 | 1.00 | 43.61 | C |
| ATOM | 404 | CG | ARG | | 68 | −22.020 | 17.138 | −73.564 | 1.00 | 46.89 | C |
| ATOM | 405 | CD | ARG | | 68 | −21.574 | 17.024 | −72.102 | 1.00 | 47.18 | C |
| ATOM | 406 | NE | ARG | | 68 | −22.579 | 16.397 | −71.244 | 1.00 | 48.41 | N |
| ATOM | 407 | CZ | ARG | | 68 | −22.528 | 16.378 | −69.911 | 1.00 | 49.06 | C |
| ATOM | 408 | NH1 | ARG | | 68 | −21.521 | 16.959 | −69.269 | 1.00 | 49.37 | N |
| ATOM | 409 | NH2 | ARG | | 68 | −23.479 | 15.767 | −69.218 | 1.00 | 47.65 | N |
| ATOM | 410 | C | ARG | | 68 | −21.561 | 20.173 | −74.173 | 1.00 | 44.90 | C |
| ATOM | 411 | O | ARG | | 68 | −20.363 | 20.144 | −73.871 | 1.00 | 46.32 | O |
| ATOM | 412 | N | ARG | | 69 | −22.013 | 20.700 | −75.307 | 1.00 | 45.24 | N |
| ATOM | 413 | CA | ARG | | 69 | −21.126 | 21.315 | −76.289 | 1.00 | 44.58 | C |
| ATOM | 414 | CB | ARG | | 69 | −21.114 | 20.489 | −77.581 | 1.00 | 47.59 | C |
| ATOM | 415 | CG | ARG | | 69 | −19.953 | 19.509 | −77.665 | 1.00 | 53.48 | C |
| ATOM | 416 | CD | ARG | | 69 | −20.302 | 18.294 | −78.514 | 1.00 | 58.03 | C |
| ATOM | 417 | NE | ARG | | 69 | −20.134 | 17.056 | −77.749 | 1.00 | 62.40 | N |
| ATOM | 418 | CZ | ARG | | 69 | −20.995 | 16.041 | −77.768 | 1.00 | 64.81 | C |
| ATOM | 419 | NH1 | ARG | | 69 | −22.092 | 16.108 | −78.517 | 1.00 | 66.14 | N |
| ATOM | 420 | NH2 | ARG | | 69 | −20.766 | 14.963 | −77.033 | 1.00 | 66.14 | N |
| ATOM | 421 | C | ARG | | 69 | −21.595 | 22.739 | −76.574 | 1.00 | 41.87 | C |
| ATOM | 422 | O | ARG | | 69 | −22.393 | 22.974 | −77.477 | 1.00 | 41.41 | O |
| ATOM | 423 | N | PRO | | 70 | −21.101 | 23.711 | −75.793 | 1.00 | 40.54 | N |
| ATOM | 424 | CD | PRO | | 70 | −20.115 | 23.533 | −74.714 | 1.00 | 40.41 | C |
| ATOM | 425 | CA | PRO | | 70 | −21.462 | 25.124 | −75.944 | 1.00 | 39.06 | C |
| ATOM | 426 | CB | PRO | | 70 | −20.842 | 25.766 | −74.706 | 1.00 | 39.39 | C |
| ATOM | 427 | CG | PRO | | 70 | −19.609 | 24.945 | −74.502 | 1.00 | 40.22 | C |
| ATOM | 428 | C | PRO | | 70 | −20.952 | 25.742 | −77.243 | 1.00 | 37.39 | C |
| ATOM | 429 | O | PRO | | 70 | −20.267 | 26.765 | −77.231 | 1.00 | 35.50 | O |
| ATOM | 430 | N | SER | | 71 | −21.314 | 25.120 | −78.362 | 1.00 | 36.41 | N |
| ATOM | 431 | CA | SER | | 71 | −20.890 | 25.589 | −79.675 | 1.00 | 36.25 | C |
| ATOM | 432 | CB | SER | | 71 | −19.486 | 25.050 | −79.970 | 1.00 | 36.36 | C |
| ATOM | 433 | OG | SER | | 71 | −19.020 | 25.486 | −81.229 | 1.00 | 39.90 | O |
| ATOM | 434 | C | SER | | 71 | −21.868 | 25.119 | −80.752 | 1.00 | 34.57 | C |
| ATOM | 435 | O | SER | | 71 | −22.451 | 24.042 | −80.632 | 1.00 | 35.34 | O |
| ATOM | 436 | N | SER | | 72 | −22.047 | 25.930 | −81.798 | 1.00 | 34.27 | N |
| ATOM | 437 | CA | SER | | 72 | −22.949 | 25.578 | −82.903 | 1.00 | 33.21 | C |
| ATOM | 438 | CB | SER | | 72 | −24.400 | 25.940 | −82.561 | 1.00 | 32.41 | C |
| ATOM | 439 | OG | SER | | 72 | −24.611 | 27.342 | −82.617 | 1.00 | 29.71 | O |
| ATOM | 440 | C | SER | | 72 | −22.546 | 26.316 | −84.175 | 1.00 | 32.55 | C |
| ATOM | 441 | O | SER | | 72 | −21.638 | 27.135 | −84.151 | 1.00 | 31.56 | O |
| ATOM | 442 | N | TRP | | 73 | −23.234 | 26.031 | −85.279 | 1.00 | 33.11 | N |
| ATOM | 443 | CA | TRP | | 73 | −22.943 | 26.683 | −86.553 | 1.00 | 32.71 | C |
| ATOM | 444 | CB | TRP | | 73 | −23.793 | 26.084 | −87.682 | 1.00 | 33.20 | C |
| ATOM | 445 | CG | TRP | | 73 | −25.265 | 26.397 | −87.568 | 1.00 | 34.29 | C |
| ATOM | 446 | CD2 | TRP | | 73 | −25.941 | 27.559 | −88.081 | 1.00 | 34.76 | C |
| ATOM | 447 | CE2 | TRP | | 73 | −27.293 | 27.456 | −87.702 | 1.00 | 34.40 | C |
| ATOM | 448 | CE3 | TRP | | 73 | −25.526 | 28.671 | −88.823 | 1.00 | 35.27 | C |
| ATOM | 449 | CD1 | TRP | | 73 | −26.215 | 25.662 | −86.918 | 1.00 | 34.16 | C |
| ATOM | 450 | NE1 | TRP | | 73 | −27.436 | 26.292 | −86.992 | 1.00 | 33.46 | N |
| ATOM | 451 | CZ2 | TRP | | 73 | −28.241 | 28.431 | −88.033 | 1.00 | 33.94 | C |
| ATOM | 452 | CZ3 | TRP | | 73 | −26.467 | 29.640 | −89.152 | 1.00 | 35.68 | C |
| ATOM | 453 | CH2 | TRP | | 73 | −27.810 | 29.511 | −88.756 | 1.00 | 35.43 | C |
| ATOM | 454 | C | TRP | | 73 | −23.212 | 28.184 | −86.479 | 1.00 | 32.61 | C |
| ATOM | 455 | O | TRP | | 73 | −22.702 | 28.954 | −87.289 | 1.00 | 33.31 | O |
| ATOM | 456 | N | ARG | | 74 | −24.019 | 28.600 | −85.505 | 1.00 | 32.46 | N |
| ATOM | 457 | CA | ARG | | 74 | −24.350 | 30.013 | −85.344 | 1.00 | 33.13 | C |
| ATOM | 458 | CB | ARG | | 74 | −25.684 | 30.192 | −84.611 | 1.00 | 32.17 | C |
| ATOM | 459 | CG | ARG | | 74 | −26.900 | 29.530 | −85.222 | 1.00 | 33.22 | C |
| ATOM | 460 | CD | ARG | | 74 | −28.157 | 30.088 | −84.555 | 1.00 | 32.21 | C |
| ATOM | 461 | NE | ARG | | 74 | −28.273 | 31.519 | −84.806 | 1.00 | 32.61 | N |
| ATOM | 462 | CZ | ARG | | 74 | −28.554 | 32.431 | −83.879 | 1.00 | 33.47 | C |
| ATOM | 463 | NH1 | ARG | | 74 | −28.758 | 32.066 | −82.623 | 1.00 | 35.15 | N |
| ATOM | 464 | NH2 | ARG | | 74 | −28.620 | 33.715 | −84.214 | 1.00 | 35.56 | N |
| ATOM | 465 | C | ARG | | 74 | −23.310 | 30.779 | −84.541 | 1.00 | 33.76 | C |
| ATOM | 466 | O | ARG | | 74 | −23.080 | 31.963 | −84.779 | 1.00 | 34.11 | O |
| ATOM | 467 | N | GLN | | 75 | −22.697 | 30.105 | −83.576 | 1.00 | 35.42 | N |
| ATOM | 468 | CA | GLN | | 75 | −21.732 | 30.759 | −82.707 | 1.00 | 37.21 | C |
| ATOM | 469 | CB | GLN | | 75 | −22.480 | 31.303 | −81.493 | 1.00 | 39.96 | C |
| ATOM | 470 | CG | GLN | | 75 | −22.041 | 32.656 | −81.007 | 1.00 | 43.37 | C |
| ATOM | 471 | CD | GLN | | 75 | −22.827 | 33.079 | −79.786 | 1.00 | 45.81 | C |
| ATOM | 472 | OE1 | GLN | | 75 | −22.491 | 34.062 | −79.128 | 1.00 | 48.68 | O |
| ATOM | 473 | NE2 | GLN | | 75 | −23.888 | 32.339 | −79.477 | 1.00 | 45.90 | N |
| ATOM | 474 | C | GLN | | 75 | −20.673 | 29.760 | −82.258 | 1.00 | 36.99 | C |
| ATOM | 475 | O | GLN | | 75 | −21.007 | 28.697 | −81.734 | 1.00 | 37.14 | O |
| ATOM | 476 | N | GLU | | 76 | −19.401 | 30.101 | −82.459 | 1.00 | 36.13 | N |
| ATOM | 477 | CA | GLU | | 76 | −18.313 | 29.201 | −82.090 | 1.00 | 36.03 | C |
| ATOM | 478 | CB | GLU | | 76 | −16.959 | 29.763 | −82.550 | 1.00 | 35.73 | C |
| ATOM | 479 | CG | GLU | | 76 | −15.834 | 28.736 | −82.476 | 1.00 | 34.73 | C |
| ATOM | 480 | CD | GLU | | 76 | −14.598 | 29.127 | −83.275 | 1.00 | 34.26 | C |
| ATOM | 481 | OE1 | GLU | | 76 | −13.695 | 28.273 | −83.400 | 1.00 | 32.60 | O |
| ATOM | 482 | OE2 | GLU | | 76 | −14.524 | 30.273 | −83.773 | 1.00 | 31.82 | O |
| ATOM | 483 | C | GLU | | 76 | −18.287 | 28.930 | −80.592 | 1.00 | 36.15 | C |
| ATOM | 484 | O | GLU | | 76 | −18.100 | 27.792 | −80.163 | 1.00 | 35.25 | O |
| ATOM | 485 | N | LYS | | 77 | −18.490 | 29.979 | −79.803 | 1.00 | 37.65 | N |
| ATOM | 486 | CA | LYS | | 77 | −18.504 | 29.868 | −78.349 | 1.00 | 39.55 | C |
| ATOM | 487 | CB | LYS | | 77 | −17.329 | 30.656 | −77.760 | 1.00 | 42.19 | C |
| ATOM | 488 | CG | LYS | | 77 | −17.245 | 30.637 | −76.241 | 1.00 | 46.02 | C |
| ATOM | 489 | CD | LYS | | 77 | −16.147 | 31.575 | −75.753 | 1.00 | 49.42 | C |
| ATOM | 490 | CE | LYS | | 77 | −16.029 | 31.568 | −74.233 | 1.00 | 50.45 | C |
| ATOM | 491 | NZ | LYS | | 77 | −15.649 | 30.226 | −73.712 | 1.00 | 52.56 | N |
| ATOM | 492 | C | LYS | | 77 | −19.824 | 30.415 | −77.795 | 1.00 | 38.19 | C |
| ATOM | 493 | O | LYS | | 77 | −20.032 | 31.628 | −77.757 | 1.00 | 38.22 | O |
| ATOM | 494 | N | ILE | | 78 | −20.713 | 29.520 | −77.375 | 1.00 | 37.80 | N |
| ATOM | 495 | CA | ILE | | 78 | −22.004 | 29.923 | −76.817 | 1.00 | 37.46 | C |
| ATOM | 496 | CB | ILE | | 78 | −23.024 | 28.748 | −76.824 | 1.00 | 37.29 | C |
| ATOM | 497 | CG2 | ILE | | 78 | −24.357 | 29.213 | −76.237 | 1.00 | 36.08 | C |
| ATOM | 498 | CG1 | ILE | | 78 | −23.216 | 28.210 | −78.244 | 1.00 | 36.10 | C |
| ATOM | 499 | CD1 | ILE | | 78 | −23.881 | 29.173 | −79.182 | 1.00 | 36.94 | C |
| ATOM | 500 | C | ILE | | 78 | −21.790 | 30.359 | −75.369 | 1.00 | 37.35 | C |
| ATOM | 501 | O | ILE | | 78 | −21.364 | 29.560 | −74.537 | 1.00 | 36.94 | O |
| ATOM | 502 | N | THR | | 79 | −22.101 | 31.616 | −75.067 | 1.00 | 38.09 | N |
| ATOM | 503 | CA | THR | | 79 | −21.920 | 32.144 | −73.718 | 1.00 | 39.79 | C |
| ATOM | 504 | CB | THR | | 79 | −21.096 | 33.439 | −73.755 | 1.00 | 39.64 | C |
| ATOM | 505 | OG1 | THR | | 79 | −21.719 | 34.378 | −74.639 | 1.00 | 41.79 | O |
| ATOM | 506 | CG2 | THR | | 79 | −19.691 | 33.153 | −74.245 | 1.00 | 42.02 | C |
| ATOM | 507 | C | THR | | 79 | −23.200 | 32.421 | −72.918 | 1.00 | 39.96 | C |
| ATOM | 508 | O | THR | | 79 | −23.148 | 32.559 | −71.693 | 1.00 | 39.73 | O |
| ATOM | 509 | N | ARG | | 80 | −24.344 | 32.508 | −73.594 | 1.00 | 38.62 | N |
| ATOM | 510 | CA | ARG | | 80 | −25.595 | 32.781 | −72.890 | 1.00 | 35.82 | C |
| ATOM | 511 | CB | ARG | | 80 | −26.777 | 32.799 | −73.871 | 1.00 | 35.18 | C |
| ATOM | 512 | CG | ARG | | 80 | −27.144 | 31.441 | −74.469 | 1.00 | 34.17 | C |
| ATOM | 513 | CD | ARG | | 80 | −28.436 | 31.533 | −75.276 | 1.00 | 31.28 | C |
| ATOM | 514 | NE | ARG | | 80 | −28.258 | 32.229 | −76.548 | 1.00 | 28.97 | N |
| ATOM | 515 | CZ | ARG | | 80 | −27.875 | 31.637 | −77.676 | 1.00 | 28.79 | C |
| ATOM | 516 | NH1 | ARG | | 80 | −27.634 | 30.330 | −77.690 | 1.00 | 27.36 | N |
| ATOM | 517 | NH2 | ARG | | 80 | −27.737 | 32.344 | −78.789 | 1.00 | 27.52 | N |
| ATOM | 518 | C | ARG | | 80 | −25.850 | 31.754 | −71.790 | 1.00 | 34.96 | C |
| ATOM | 519 | O | ARG | | 80 | −25.455 | 30.593 | −71.905 | 1.00 | 33.89 | O |
| ATOM | 520 | N | THR | | 81 | −26.506 | 32.190 | −70.717 | 1.00 | 35.04 | N |
| ATOM | 521 | CA | THR | | 81 | −26.821 | 31.310 | −69.594 | 1.00 | 35.19 | C |
| ATOM | 522 | CB | THR | | 81 | −27.054 | 32.130 | −68.311 | 1.00 | 36.24 | C |
| ATOM | 523 | OG1 | THR | | 81 | −28.244 | 32.924 | −68.456 | 1.00 | 38.15 | O |
| ATOM | 524 | CG2 | THR | | 81 | −25.874 | 33.056 | −68.054 | 1.00 | 37.03 | C |
| ATOM | 525 | C | THR | | 81 | −28.086 | 30.503 | −69.891 | 1.00 | 34.54 | C |
| ATOM | 526 | O | THR | | 81 | −28.806 | 30.813 | −70.839 | 1.00 | 33.46 | O |
| ATOM | 527 | N | LYS | | 82 | −28.357 | 29.472 | −69.089 | 1.00 | 34.65 | N |
| ATOM | 528 | CA | LYS | | 82 | −29.559 | 28.657 | −69.280 | 1.00 | 36.61 | C |
| ATOM | 529 | CB | LYS | | 82 | −29.697 | 27.585 | −68.197 | 1.00 | 39.22 | C |
| ATOM | 530 | CG | LYS | | 82 | −28.609 | 26.542 | −68.162 | 1.00 | 44.85 | C |
| ATOM | 531 | CD | LYS | | 82 | −29.042 | 25.344 | −67.316 | 1.00 | 48.00 | C |
| ATOM | 532 | CE | LYS | | 82 | −29.418 | 25.752 | −65.894 | 1.00 | 50.16 | C |
| ATOM | 533 | NZ | LYS | | 82 | −29.850 | 24.579 | −65.075 | 1.00 | 51.59 | N |
| ATOM | 534 | C | LYS | | 82 | −30.793 | 29.540 | −69.194 | 1.00 | 36.39 | C |
| ATOM | 535 | O | LYS | | 82 | −31.766 | 29.345 | −69.922 | 1.00 | 36.10 | O |
| ATOM | 536 | N | GLU | | 83 | −30.748 | 30.496 | −68.276 | 1.00 | 35.53 | N |
| ATOM | 537 | CA | GLU | | 83 | −31.860 | 31.407 | −68.062 | 1.00 | 35.08 | C |
| ATOM | 538 | CB | GLU | | 83 | −31.589 | 32.277 | −66.827 | 1.00 | 36.60 | C |
| ATOM | 539 | CG | GLU | | 83 | −31.600 | 31.494 | −65.507 | 1.00 | 38.74 | C |
| ATOM | 540 | CD | GLU | | 83 | −30.382 | 30.586 | −65.320 | 1.00 | 40.94 | C |
| ATOM | 541 | OE1 | GLU | | 83 | −30.505 | 29.566 | −64.603 | 1.00 | 42.51 | O |
| ATOM | 542 | OE2 | GLU | | 83 | −29.300 | 30.896 | −65.867 | 1.00 | 40.30 | O |
| ATOM | 543 | C | GLU | | 83 | −32.127 | 32.278 | −69.282 | 1.00 | 33.29 | C |
| ATOM | 544 | O | GLU | | 83 | −33.282 | 32.503 | −69.647 | 1.00 | 31.45 | O |
| ATOM | 545 | N | GLU | | 84 | −31.068 | 32.769 | −69.913 | 1.00 | 33.06 | N |
| ATOM | 546 | CA | GLU | | 84 | −31.227 | 33.607 | −71.099 | 1.00 | 32.42 | C |
| ATOM | 547 | CB | GLU | | 84 | −29.898 | 34.254 | −71.478 | 1.00 | 34.53 | C |
| ATOM | 548 | CG | GLU | | 84 | −29.322 | 35.141 | −70.392 | 1.00 | 38.11 | C |
| ATOM | 549 | CD | GLU | | 84 | −27.951 | 35.669 | −70.750 | 1.00 | 38.58 | C |
| ATOM | 550 | OE1 | GLU | | 84 | −27.067 | 34.849 | −71.078 | 1.00 | 40.41 | O |
| ATOM | 551 | OE2 | GLU | | 84 | −27.755 | 36.902 | −70.701 | 1.00 | 39.09 | O |
| ATOM | 552 | C | GLU | | 84 | −31.735 | 32.757 | −72.256 | 1.00 | 31.30 | C |
| ATOM | 553 | O | GLU | | 84 | −32.508 | 33.226 | −73.091 | 1.00 | 29.28 | O |
| ATOM | 554 | N | ALA | | 85 | −31.292 | 31.505 | −72.295 | 1.00 | 29.62 | N |
| ATOM | 555 | CA | ALA | | 85 | −31.704 | 30.571 | −73.333 | 1.00 | 28.65 | C |
| ATOM | 556 | CB | ALA | | 85 | −30.912 | 29.273 | −73.210 | 1.00 | 29.16 | C |
| ATOM | 557 | C | ALA | | 85 | −33.197 | 30.286 | −73.198 | 1.00 | 28.26 | C |
| ATOM | 558 | O | ALA | | 85 | −33.924 | 30.248 | −74.192 | 1.00 | 25.90 | O |
| ATOM | 559 | N | LEU | | 86 | −33.652 | 30.078 | −71.965 | 1.00 | 26.44 | N |
| ATOM | 560 | CA | LEU | | 86 | −35.066 | 29.801 | −71.731 | 1.00 | 27.63 | C |
| ATOM | 561 | CB | LEU | | 86 | −35.316 | 29.446 | −70.261 | 1.00 | 25.67 | C |
| ATOM | 562 | CG | LEU | | 86 | −36.772 | 29.120 | −69.885 | 1.00 | 26.59 | C |
| ATOM | 563 | CD1 | LEU | | 86 | −37.312 | 28.003 | −70.777 | 1.00 | 23.47 | C |
| ATOM | 564 | CD2 | LEU | | 86 | −36.844 | 28.707 | −68.422 | 1.00 | 25.60 | C |
| ATOM | 565 | C | LEU | | 86 | −35.920 | 31.003 | −72.130 | 1.00 | 26.37 | C |
| ATOM | 566 | O | LEU | | 86 | −37.036 | 30.839 | −72.621 | 1.00 | 25.81 | O |
| ATOM | 567 | N | GLU | | 87 | −35.396 | 32.212 | −71.926 | 1.00 | 27.04 | N |
| ATOM | 568 | CA | GLU | | 87 | −36.144 | 33.408 | −72.297 | 1.00 | 29.41 | C |
| ATOM | 569 | CB | GLU | | 87 | −35.503 | 34.666 | −71.702 | 1.00 | 32.96 | C |
| ATOM | 570 | CG | GLU | | 87 | −35.826 | 34.831 | −70.228 | 1.00 | 40.07 | C |
| ATOM | 571 | CD | GLU | | 87 | −37.314 | 34.610 | −69.941 | 1.00 | 44.94 | C |
| ATOM | 572 | OE1 | GLU | | 87 | −38.144 | 35.414 | −70.425 | 1.00 | 46.00 | O |
| ATOM | 573 | OE2 | GLU | | 87 | −37.652 | 33.627 | −69.240 | 1.00 | 47.31 | O |
| ATOM | 574 | C | GLU | | 87 | −36.276 | 33.545 | −73.806 | 1.00 | 28.57 | C |
| ATOM | 575 | O | GLU | | 87 | −37.298 | 34.025 | −74.302 | 1.00 | 27.18 | O |
| ATOM | 576 | N | LEU | | 88 | −35.240 | 33.139 | −74.530 | 1.00 | 26.02 | N |
| ATOM | 577 | CA | LEU | | 88 | −35.265 | 33.184 | −75.985 | 1.00 | 24.59 | C |
| ATOM | 578 | CB | LEU | | 88 | −33.890 | 32.836 | −76.555 | 1.00 | 25.51 | C |
| ATOM | 579 | CG | LEU | | 88 | −32.819 | 33.927 | −76.454 | 1.00 | 26.64 | C |
| ATOM | 580 | CD1 | LEU | | 88 | −31.449 | 33.351 | −76.812 | 1.00 | 25.81 | C |
| ATOM | 581 | CD2 | LEU | | 88 | −33.192 | 35.080 | −77.379 | 1.00 | 26.30 | C |
| ATOM | 582 | C | LEU | | 88 | −36.295 | 32.162 | −76.463 | 1.00 | 23.40 | C |
| ATOM | 583 | O | LEU | | 88 | −37.129 | 32.463 | −77.316 | 1.00 | 24.39 | O |
| ATOM | 584 | N | ILE | | 89 | −36.228 | 30.958 | −75.905 | 1.00 | 20.40 | N |
| ATOM | 585 | CA | ILE | | 89 | −37.156 | 29.889 | −76.257 | 1.00 | 21.30 | C |
| ATOM | 586 | CB | ILE | | 89 | −36.861 | 28.600 | −75.437 | 1.00 | 20.83 | C |
| ATOM | 587 | CG2 | ILE | | 89 | −38.087 | 27.697 | −75.406 | 1.00 | 19.00 | C |
| ATOM | 588 | CG1 | ILE | | 89 | −35.671 | 27.842 | −76.042 | 1.00 | 18.75 | C |
| ATOM | 589 | CD1 | ILE | | 89 | −36.017 | 27.018 | −77.298 | 1.00 | 17.72 | C |
| ATOM | 590 | C | ILE | | 89 | −38.586 | 30.352 | −75.990 | 1.00 | 22.69 | C |
| ATOM | 591 | O | ILE | | 89 | −39.467 | 30.170 | −76.830 | 1.00 | 24.07 | O |
| ATOM | 592 | N | ASN | | 90 | −38.819 | 30.959 | −74.827 | 1.00 | 23.22 | N |
| ATOM | 593 | CA | ASN | | 90 | −40.161 | 31.429 | −74.493 | 1.00 | 22.69 | C |
| ATOM | 594 | CB | ASN | | 90 | −40.215 | 32.012 | −73.077 | 1.00 | 23.78 | C |
| ATOM | 595 | CG | ASN | | 90 | −40.123 | 30.942 | −71.995 | 1.00 | 24.09 | C |
| ATOM | 596 | OD1 | ASN | | 90 | −40.502 | 29.790 | −72.208 | 1.00 | 25.66 | O |
| ATOM | 597 | ND2 | ASN | | 90 | −39.641 | 31.332 | −70.822 | 1.00 | 25.94 | N |
| ATOM | 598 | C | ASN | | 90 | −40.626 | 32.476 | −75.496 | 1.00 | 22.56 | C |
| ATOM | 599 | O | ASN | | 90 | −41.798 | 32.493 | −75.880 | 1.00 | 22.81 | O |
| ATOM | 600 | N | GLY | | 91 | −39.708 | 33.344 | −75.920 | 1.00 | 22.08 | N |
| ATOM | 601 | CA | GLY | | 91 | −40.045 | 34.369 | −76.893 | 1.00 | 21.04 | C |
| ATOM | 602 | C | GLY | | 91 | −40.428 | 33.775 | −78.240 | 1.00 | 22.57 | C |
| ATOM | 603 | O | GLY | | 91 | −41.375 | 34.234 | −78.890 | 1.00 | 23.12 | O |
| ATOM | 604 | N | TYR | | 92 | −39.695 | 32.748 | −78.671 | 1.00 | 21.06 | N |
| ATOM | 605 | CA | TYR | | 92 | −39.973 | 32.095 | −79.945 | 1.00 | 19.79 | C |
| ATOM | 606 | CB | TYR | | 92 | −38.895 | 31.053 | −80.268 | 1.00 | 20.25 | C |
| ATOM | 607 | CG | TYR | | 92 | −37.502 | 31.621 | −80.494 | 1.00 | 22.09 | C |
| ATOM | 608 | CD1 | TYR | | 92 | −36.372 | 30.897 | −80.120 | 1.00 | 22.58 | C |
| ATOM | 609 | CE1 | TYR | | 92 | −35.092 | 31.403 | −80.308 | 1.00 | 24.75 | C |
| ATOM | 610 | CD2 | TYR | | 92 | −37.315 | 32.874 | −81.071 | 1.00 | 22.30 | C |
| ATOM | 611 | CE2 | TYR | | 92 | −36.029 | 33.393 | −81.266 | 1.00 | 24.63 | C |
| ATOM | 612 | CZ | TYR | | 92 | −34.926 | 32.647 | −80.876 | 1.00 | 24.91 | C |
| ATOM | 613 | OH | TYR | | 92 | −33.651 | 33.150 | −81.030 | 1.00 | 28.01 | O |
| ATOM | 614 | C | TYR | | 92 | −41.334 | 31.408 | −79.899 | 1.00 | 18.33 | C |
| ATOM | 615 | O | TYR | | 92 | −42.065 | 31.421 | −80.880 | 1.00 | 18.35 | O |
| ATOM | 616 | N | ILE | | 93 | −41.668 | 30.806 | −78.760 | 1.00 | 18.81 | N |
| ATOM | 617 | CA | ILE | | 93 | −42.948 | 30.116 | −78.609 | 1.00 | 19.17 | C |
| ATOM | 618 | CB | ILE | | 93 | −43.036 | 29.381 | −77.247 | 1.00 | 19.71 | C |
| ATOM | 619 | CG2 | ILE | | 93 | −44.472 | 28.921 | −76.981 | 1.00 | 17.96 | C |
| ATOM | 620 | CG1 | ILE | | 93 | −42.099 | 28.167 | −77.246 | 1.00 | 18.40 | C |
| ATOM | 621 | CD1 | ILE | | 93 | −42.116 | 27.401 | −75.936 | 1.00 | 19.85 | C |
| ATOM | 622 | C | ILE | | 93 | −44.091 | 31.126 | −78.712 | 1.00 | 20.54 | C |
| ATOM | 623 | O | ILE | | 93 | −45.125 | 30.857 | −79.327 | 1.00 | 20.59 | O |
| ATOM | 624 | N | GLN | | 94 | −43.895 | 32.292 | −78.109 | 1.00 | 20.79 | N |
| ATOM | 625 | CA | GLN | | 94 | −44.904 | 33.338 | −78.150 | 1.00 | 23.15 | C |
| ATOM | 626 | CB | GLN | | 94 | −44.467 | 34.535 | −77.301 | 1.00 | 24.71 | C |
| ATOM | 627 | CG | GLN | | 94 | −44.469 | 34.247 | −75.811 | 1.00 | 28.57 | C |
| ATOM | 628 | CD | GLN | | 94 | −44.353 | 35.504 | −74.981 | 1.00 | 33.05 | C |
| ATOM | 629 | OE1 | GLN | | 94 | −43.352 | 36.213 | −75.044 | 1.00 | 35.55 | O |
| ATOM | 630 | NE2 | GLN | | 94 | −45.386 | 35.789 | −74.201 | 1.00 | 36.40 | N |
| ATOM | 631 | C | GLN | | 94 | −45.167 | 33.792 | −79.581 | 1.00 | 23.38 | C |
| ATOM | 632 | O | GLN | | 94 | −46.322 | 33.935 | −79.994 | 1.00 | 23.82 | O |
| ATOM | 633 | N | LYS | | 95 | −44.097 | 34.012 | −80.336 | 1.00 | 23.21 | N |
| ATOM | 634 | CA | LYS | | 95 | −44.220 | 34.454 | −81.719 | 1.00 | 24.19 | C |
| ATOM | 635 | CB | LYS | | 95 | −42.847 | 34.887 | −82.246 | 1.00 | 28.16 | C |
| ATOM | 636 | CG | LYS | | 95 | −42.427 | 36.265 | −81.733 | 1.00 | 34.00 | C |
| ATOM | 637 | CD | LYS | | 95 | −40.944 | 36.570 | −81.949 | 1.00 | 39.23 | C |
| ATOM | 638 | CE | LYS | | 95 | −40.070 | 35.856 | −80.911 | 1.00 | 43.92 | C |
| ATOM | 639 | NZ | LYS | | 95 | −38.651 | 36.344 | −80.864 | 1.00 | 42.61 | N |
| ATOM | 640 | C | LYS | | 95 | −44.840 | 33.384 | −82.619 | 1.00 | 22.76 | C |
| ATOM | 641 | O | LYS | | 95 | −45.509 | 33.695 | −83.601 | 1.00 | 20.97 | O |
| ATOM | 642 | N | ILE | | 96 | −44.618 | 32.118 | −82.286 | 1.00 | 20.58 | N |
| ATOM | 643 | CA | ILE | | 96 | −45.190 | 31.035 | −83.080 | 1.00 | 20.56 | C |
| ATOM | 644 | CB | ILE | | 96 | −44.504 | 29.689 | −82.750 | 1.00 | 18.89 | C |
| ATOM | 645 | CG2 | ILE | | 96 | −45.295 | 28.522 | −83.342 | 1.00 | 18.25 | C |
| ATOM | 646 | CG1 | ILE | | 96 | −43.063 | 29.712 | −83.270 | 1.00 | 18.81 | C |
| ATOM | 647 | CD1 | ILE | | 96 | −42.230 | 28.495 | −82.866 | 1.00 | 21.84 | C |
| ATOM | 648 | C | ILE | | 96 | −46.689 | 30.942 | −82.784 | 1.00 | 19.80 | C |
| ATOM | 649 | O | ILE | | 96 | −47.517 | 30.847 | −83.697 | 1.00 | 19.75 | O |
| ATOM | 650 | N | LYS | | 97 | −47.036 | 30.986 | −81.505 | 1.00 | 19.01 | N |
| ATOM | 651 | CA | LYS | | 97 | −48.436 | 30.903 | −81.093 | 1.00 | 20.09 | C |
| ATOM | 652 | CB | LYS | | 97 | −48.544 | 30.769 | −79.574 | 1.00 | 20.11 | C |
| ATOM | 653 | CG | LYS | | 97 | −48.196 | 29.394 | −79.053 | 1.00 | 22.03 | C |
| ATOM | 654 | CD | LYS | | 97 | −48.661 | 29.217 | −77.616 | 1.00 | 22.66 | C |
| ATOM | 655 | CE | LYS | | 97 | −48.440 | 27.781 | −77.169 | 1.00 | 23.25 | C |
| ATOM | 656 | NZ | LYS | | 97 | −49.076 | 27.472 | −75.860 | 1.00 | 22.54 | N |
| ATOM | 657 | C | LYS | | 97 | −49.279 | 32.092 | −81.543 | 1.00 | 19.51 | C |
| ATOM | 658 | O | LYS | | 97 | −50.478 | 31.952 | −81.785 | 1.00 | 19.03 | O |
| ATOM | 659 | N | SER | | 98 | −48.660 | 33.261 | −81.647 | 1.00 | 19.97 | N |
| ATOM | 660 | CA | SER | | 98 | −49.381 | 34.459 | −82.071 | 1.00 | 21.29 | C |
| ATOM | 661 | CB | SER | | 98 | −48.656 | 35.713 | −81.587 | 1.00 | 20.80 | C |
| ATOM | 662 | OG | SER | | 98 | −47.423 | 35.857 | −82.265 | 1.00 | 24.27 | O |
| ATOM | 663 | C | SER | | 98 | −49.489 | 34.506 | −83.588 | 1.00 | 22.65 | C |
| ATOM | 664 | O | SER | | 98 | −50.297 | 35.255 | −84.144 | 1.00 | 22.70 | O |
| ATOM | 665 | N | GLY | | 99 | −48.668 | 33.705 | −84.262 | 1.00 | 22.03 | N |
| ATOM | 666 | CA | GLY | | 99 | −48.689 | 33.689 | −85.710 | 1.00 | 22.11 | C |
| ATOM | 667 | C | GLY | | 99 | −47.780 | 34.740 | −86.321 | 1.00 | 24.34 | C |
| ATOM | 668 | O | GLY | | 99 | −47.726 | 34.880 | −87.541 | 1.00 | 22.82 | O |
| ATOM | 669 | N | GLU | | 100 | −47.069 | 35.488 | −85.476 | 1.00 | 25.40 | N |
| ATOM | 670 | CA | GLU | | 100 | −46.153 | 36.528 | −85.947 | 1.00 | 28.95 | C |
| ATOM | 671 | CB | GLU | | 100 | −45.551 | 37.283 | −84.759 | 1.00 | 32.53 | C |
| ATOM | 672 | CG | GLU | | 100 | −46.540 | 38.194 | −84.026 | 1.00 | 41.15 | C |
| ATOM | 673 | CD | GLU | | 100 | −45.934 | 38.857 | −82.789 | 1.00 | 44.77 | C |
| ATOM | 674 | OE1 | GLU | | 100 | −45.741 | 38.169 | −81.762 | 1.00 | 46.13 | O |
| ATOM | 675 | OE2 | GLU | | 100 | −45.647 | 40.073 | −82.848 | 1.00 | 49.70 | O |
| ATOM | 676 | C | GLU | | 100 | −45.031 | 35.923 | −86.783 | 1.00 | 29.40 | C |
| ATOM | 677 | O | GLU | | 100 | −44.581 | 36.509 | −87.768 | 1.00 | 28.16 | O |
| ATOM | 678 | N | GLU | | 101 | −44.592 | 34.737 | −86.378 | 1.00 | 28.86 | N |
| ATOM | 679 | CA | GLU | | 101 | −43.523 | 34.017 | −87.055 | 1.00 | 30.92 | C |
| ATOM | 680 | CB | GLU | | 101 | −42.198 | 34.254 | −86.328 | 1.00 | 35.00 | C |
| ATOM | 681 | CG | GLU | | 101 | −41.529 | 35.573 | −86.639 | 1.00 | 41.45 | C |
| ATOM | 682 | CD | GLU | | 101 | −40.756 | 35.515 | −87.935 | 1.00 | 44.31 | C |
| ATOM | 683 | OE1 | GLU | | 101 | −41.382 | 35.274 | −88.989 | 1.00 | 48.34 | O |
| ATOM | 684 | OE2 | GLU | | 101 | −39.521 | 35.700 | −87.901 | 1.00 | 47.67 | O |
| ATOM | 685 | C | GLU | | 101 | −43.859 | 32.536 | −87.000 | 1.00 | 30.84 | C |
| ATOM | 686 | O | GLU | | 101 | −44.427 | 32.066 | −86.014 | 1.00 | 30.96 | O |
| ATOM | 687 | N | ASP | | 102 | −43.532 | 31.785 | −88.045 | 1.00 | 28.18 | N |
| ATOM | 688 | CA | ASP | | 102 | −43.825 | 30.369 | −87.967 | 1.00 | 29.28 | C |
| ATOM | 689 | CB | ASP | | 102 | −44.466 | 29.849 | −89.265 | 1.00 | 33.40 | C |
| ATOM | 690 | CG | ASP | | 102 | −43.661 | 30.160 | −90.489 | 1.00 | 37.39 | C |
| ATOM | 691 | OD1 | ASP | | 102 | −44.247 | 30.119 | −91.598 | 1.00 | 38.22 | O |
| ATOM | 692 | OD2 | ASP | | 102 | −42.451 | 30.435 | −90.350 | 1.00 | 41.69 | O |
| ATOM | 693 | C | ASP | | 102 | −42.585 | 29.570 | −87.571 | 1.00 | 26.83 | C |
| ATOM | 694 | O | ASP | | 102 | −41.449 | 30.016 | −87.740 | 1.00 | 24.79 | O |
| ATOM | 695 | N | PHE | | 103 | −42.831 | 28.409 | −86.993 | 1.00 | 24.22 | N |
| ATOM | 696 | CA | PHE | | 103 | −41.784 | 27.526 | −86.514 | 1.00 | 24.56 | C |
| ATOM | 697 | CB | PHE | | 103 | −42.391 | 26.157 | −86.202 | 1.00 | 25.53 | C |
| ATOM | 698 | CG | PHE | | 103 | −41.408 | 25.187 | −85.632 | 1.00 | 27.92 | C |
| ATOM | 699 | CD1 | PHE | | 103 | −41.200 | 25.118 | −84.262 | 1.00 | 29.19 | C |
| ATOM | 700 | CD2 | PHE | | 103 | −40.638 | 24.392 | −86.468 | 1.00 | 29.19 | C |
| ATOM | 701 | CE1 | PHE | | 103 | −40.241 | 24.272 | −83.737 | 1.00 | 28.53 | C |
| ATOM | 702 | CE2 | PHE | | 103 | −39.678 | 23.546 | −85.949 | 1.00 | 29.70 | C |
| ATOM | 703 | CZ | PHE | | 103 | −39.479 | 23.488 | −84.581 | 1.00 | 29.29 | C |
| ATOM | 704 | C | PHE | | 103 | −40.587 | 27.344 | −87.452 | 1.00 | 23.64 | C |
| ATOM | 705 | O | PHE | | 103 | −39.433 | 27.528 | −87.051 | 1.00 | 22.12 | O |
| ATOM | 706 | N | GLU | | 104 | −40.868 | 26.964 | −88.692 | 1.00 | 23.35 | N |
| ATOM | 707 | CA | GLU | | 104 | −39.820 | 26.713 | −89.682 | 1.00 | 24.68 | C |
| ATOM | 708 | CB | GLU | | 104 | −40.446 | 26.321 | −91.027 | 1.00 | 24.58 | C |
| ATOM | 709 | CG | GLU | | 104 | −41.215 | 25.017 | −90.988 | 1.00 | 24.92 | C |
| ATOM | 710 | CD | GLU | | 104 | −42.664 | 25.201 | −90.592 | 1.00 | 27.46 | C |
| ATOM | 711 | OE1 | GLU | | 104 | −43.004 | 26.236 | −89.965 | 1.00 | 22.43 | O |
| ATOM | 712 | OE2 | GLU | | 104 | −43.462 | 24.296 | −90.910 | 1.00 | 30.49 | O |
| ATOM | 713 | C | GLU | | 104 | −38.882 | 27.888 | −89.887 | 1.00 | 24.17 | C |
| ATOM | 714 | O | GLU | | 104 | −37.665 | 27.714 | −90.027 | 1.00 | 24.38 | O |
| ATOM | 715 | N | SER | | 105 | −39.460 | 29.085 | −89.903 | 1.00 | 23.52 | N |
| ATOM | 716 | CA | SER | | 105 | −38.704 | 30.310 | −90.095 | 1.00 | 23.54 | C |
| ATOM | 717 | CB | SER | | 105 | −39.674 | 31.483 | −90.242 | 1.00 | 25.75 | C |
| ATOM | 718 | OG | SER | | 105 | −38.983 | 32.704 | −90.361 | 1.00 | 27.97 | O |
| ATOM | 719 | C | SER | | 105 | −37.724 | 30.583 | −88.950 | 1.00 | 22.93 | C |
| ATOM | 720 | O | SER | | 105 | −36.550 | 30.882 | −89.180 | 1.00 | 23.53 | O |
| ATOM | 721 | N | LEU | | 106 | −38.206 | 30.485 | −87.715 | 1.00 | 22.10 | N |
| ATOM | 722 | CA | LEU | | 106 | −37.366 | 30.729 | −86.544 | 1.00 | 21.00 | C |
| ATOM | 723 | CB | LEU | | 106 | −38.231 | 30.813 | −85.281 | 1.00 | 19.48 | C |
| ATOM | 724 | CG | LEU | | 106 | −39.255 | 31.951 | −85.272 | 1.00 | 20.14 | C |
| ATOM | 725 | CD1 | LEU | | 106 | −40.108 | 31.889 | −84.001 | 1.00 | 17.01 | C |
| ATOM | 726 | CD2 | LEU | | 106 | −38.517 | 33.285 | −85.363 | 1.00 | 17.02 | C |
| ATOM | 727 | C | LEU | | 106 | −36.304 | 29.646 | −86.374 | 1.00 | 19.92 | C |
| ATOM | 728 | O | LEU | | 106 | −35.207 | 29.918 | −85.892 | 1.00 | 18.98 | O |
| ATOM | 729 | N | ALA | | 107 | −36.630 | 28.420 | −86.765 | 1.00 | 19.37 | N |
| ATOM | 730 | CA | ALA | | 107 | −35.682 | 27.312 | −86.655 | 1.00 | 20.71 | C |
| ATOM | 731 | CB | ALA | | 107 | −36.382 | 25.995 | −86.948 | 1.00 | 17.40 | C |
| ATOM | 732 | C | ALA | | 107 | −34.511 | 27.503 | −87.618 | 1.00 | 20.55 | C |
| ATOM | 733 | O | ALA | | 107 | −33.353 | 27.298 | −87.251 | 1.00 | 20.24 | O |
| ATOM | 734 | N | SER | | 108 | −34.820 | 27.888 | −88.851 | 1.00 | 20.75 | N |
| ATOM | 735 | CA | SER | | 108 | −33.799 | 28.115 | −89.868 | 1.00 | 21.17 | C |
| ATOM | 736 | CB | SER | | 108 | −34.446 | 28.521 | −91.194 | 1.00 | 21.17 | C |
| ATOM | 737 | OG | SER | | 108 | −35.204 | 27.457 | −91.740 | 1.00 | 26.99 | O |
| ATOM | 738 | C | SER | | 108 | −32.867 | 29.237 | −89.428 | 1.00 | 22.83 | C |
| ATOM | 739 | O | SER | | 108 | −31.657 | 29.197 | −89.662 | 1.00 | 22.76 | O |
| ATOM | 740 | N | GLN | | 109 | −33.457 | 30.239 | −88.795 | 1.00 | 23.07 | N |
| ATOM | 741 | CA | GLN | | 109 | −32.729 | 31.410 | −88.336 | 1.00 | 24.82 | C |
| ATOM | 742 | CB | GLN | | 109 | −33.686 | 32.596 | −88.224 | 1.00 | 26.15 | C |
| ATOM | 743 | CG | GLN | | 109 | −34.057 | 33.260 | −89.526 | 1.00 | 27.56 | C |
| ATOM | 744 | CD | GLN | | 109 | −35.099 | 34.338 | −89.328 | 1.00 | 28.78 | C |
| ATOM | 745 | OE1 | GLN | | 109 | −36.304 | 34.071 | −89.360 | 1.00 | 32.49 | O |
| ATOM | 746 | NE2 | GLN | | 109 | −34.644 | 35.562 | −89.100 | 1.00 | 28.95 | N |
| ATOM | 747 | C | GLN | | 109 | −31.999 | 31.293 | −87.011 | 1.00 | 24.67 | C |
| ATOM | 748 | O | GLN | | 109 | −30.868 | 31.751 | −86.886 | 1.00 | 24.49 | O |
| ATOM | 749 | N | PHE | | 110 | −32.634 | 30.671 | −86.024 | 1.00 | 24.93 | N |
| ATOM | 750 | CA | PHE | | 110 | −32.043 | 30.636 | −84.697 | 1.00 | 24.35 | C |
| ATOM | 751 | CB | PHE | | 110 | −32.942 | 31.462 | −83.763 | 1.00 | 24.01 | C |
| ATOM | 752 | CG | PHE | | 110 | −33.304 | 32.821 | −84.326 | 1.00 | 24.93 | C |
| ATOM | 753 | CD1 | PHE | | 110 | −34.613 | 33.117 | −84.683 | 1.00 | 25.09 | C |
| ATOM | 754 | CD2 | PHE | | 110 | −32.324 | 33.787 | −84.530 | 1.00 | 26.39 | C |
| ATOM | 755 | CE1 | PHE | | 110 | −34.947 | 34.354 | −85.239 | 1.00 | 25.11 | C |
| ATOM | 756 | CE2 | PHE | | 110 | −32.644 | 35.030 | −85.087 | 1.00 | 26.05 | C |
| ATOM | 757 | CZ | PHE | | 110 | −33.961 | 35.315 | −85.442 | 1.00 | 26.01 | C |
| ATOM | 758 | C | PHE | | 110 | −31.686 | 29.320 | −84.018 | 1.00 | 23.51 | C |
| ATOM | 759 | O | PHE | | 110 | −31.101 | 29.338 | −82.939 | 1.00 | 25.94 | O |
| ATOM | 760 | N | SER | | 111 | −32.011 | 28.184 | −84.617 | 1.00 | 22.88 | N |
| ATOM | 761 | CA | SER | | 111 | −31.687 | 26.921 | −83.969 | 1.00 | 22.83 | C |
| ATOM | 762 | CB | SER | | 111 | −32.401 | 25.768 | −84.656 | 1.00 | 20.14 | C |
| ATOM | 763 | OG | SER | | 111 | −32.137 | 24.561 | −83.968 | 1.00 | 22.59 | O |
| ATOM | 764 | C | SER | | 111 | −30.185 | 26.642 | −83.951 | 1.00 | 24.68 | C |
| ATOM | 765 | O | SER | | 111 | −29.515 | 26.765 | −84.974 | 1.00 | 22.64 | O |
| ATOM | 766 | N | ASP | | 112 | −29.667 | 26.262 | −82.783 | 1.00 | 25.44 | N |
| ATOM | 767 | CA | ASP | | 112 | −28.244 | 25.964 | −82.631 | 1.00 | 26.82 | C |
| ATOM | 768 | CB | ASP | | 112 | −27.791 | 26.208 | −81.186 | 1.00 | 24.69 | C |
| ATOM | 769 | CG | ASP | | 112 | −27.570 | 27.680 | −80.887 | 1.00 | 25.32 | C |
| ATOM | 770 | OD1 | ASP | | 112 | −26.863 | 28.340 | −81.675 | 1.00 | 23.55 | O |
| ATOM | 771 | OD2 | ASP | | 112 | −28.088 | 28.179 | −79.864 | 1.00 | 25.17 | O |
| ATOM | 772 | C | ASP | | 112 | −27.899 | 24.536 | −83.030 | 1.00 | 27.96 | C |
| ATOM | 773 | O | ASP | | 112 | −26.989 | 23.931 | −82.473 | 1.00 | 30.66 | O |
| ATOM | 774 | N | CYS | | 113 | −28.633 | 24.004 | −84.002 | 1.00 | 28.66 | N |
| ATOM | 775 | CA | CYS | | 113 | −28.398 | 22.655 | −84.495 | 1.00 | 28.76 | C |
| ATOM | 776 | CB | CYS | | 113 | −29.511 | 21.726 | −84.021 | 1.00 | 28.26 | C |
| ATOM | 777 | SG | CYS | | 113 | −29.415 | 20.057 | −84.682 | 1.00 | 36.66 | S |
| ATOM | 778 | C | CYS | | 113 | −28.373 | 22.717 | −86.016 | 1.00 | 27.51 | C |
| ATOM | 779 | O | CYS | | 113 | −29.103 | 23.503 | −86.615 | 1.00 | 29.37 | O |
| ATOM | 780 | N | SER | | 114 | −27.529 | 21.901 | −86.641 | 1.00 | 25.10 | N |
| ATOM | 781 | CA | SER | | 114 | −27.428 | 21.901 | −88.097 | 1.00 | 25.86 | C |
| ATOM | 782 | CB | SER | | 114 | −26.321 | 20.940 | −88.562 | 1.00 | 26.12 | C |
| ATOM | 783 | OG | SER | | 114 | −26.490 | 19.645 | −88.009 | 1.00 | 32.53 | O |
| ATOM | 784 | C | SER | | 114 | −28.752 | 21.544 | −88.768 | 1.00 | 24.32 | C |
| ATOM | 785 | O | SER | | 114 | −28.940 | 21.802 | −89.958 | 1.00 | 24.25 | O |
| ATOM | 786 | N | SER | | 115 | −29.671 | 20.955 | −88.005 | 1.00 | 23.86 | N |
| ATOM | 787 | CA | SER | | 115 | −30.980 | 20.593 | −88.545 | 1.00 | 23.37 | C |
| ATOM | 788 | CB | SER | | 115 | −31.797 | 19.822 | −87.504 | 1.00 | 24.93 | C |
| ATOM | 789 | OG | SER | | 115 | −31.954 | 20.577 | −86.318 | 1.00 | 26.09 | O |
| ATOM | 790 | C | SER | | 115 | −31.713 | 21.868 | −88.950 | 1.00 | 22.22 | C |
| ATOM | 791 | O | SER | | 115 | −32.768 | 21.825 | −89.583 | 1.00 | 21.06 | O |
| ATOM | 792 | N | ALA | | 116 | −31.136 | 23.004 | −88.577 | 1.00 | 20.37 | N |
| ATOM | 793 | CA | ALA | | 116 | −31.701 | 24.303 | −88.911 | 1.00 | 20.24 | C |
| ATOM | 794 | CB | ALA | | 116 | −30.862 | 25.415 | −88.277 | 1.00 | 21.48 | C |
| ATOM | 795 | C | ALA | | 116 | −31.748 | 24.493 | −90.434 | 1.00 | 22.31 | C |
| ATOM | 796 | O | ALA | | 116 | −32.649 | 25.158 | −90.957 | 1.00 | 20.99 | O |
| ATOM | 797 | N | LYS | | 117 | −30.781 | 23.909 | −91.138 | 1.00 | 20.32 | N |
| ATOM | 798 | CA | LYS | | 117 | −30.715 | 24.048 | −92.593 | 1.00 | 22.01 | C |
| ATOM | 799 | CB | LYS | | 117 | −29.373 | 23.532 | −93.126 | 1.00 | 25.14 | C |
| ATOM | 800 | CG | LYS | | 117 | −28.166 | 24.091 | −92.404 | 1.00 | 28.67 | C |
| ATOM | 801 | CD | LYS | | 117 | −28.207 | 25.606 | −92.338 | 1.00 | 31.87 | C |
| ATOM | 802 | CE | LYS | | 117 | −27.120 | 26.139 | −91.407 | 1.00 | 34.51 | C |
| ATOM | 803 | NZ | LYS | | 117 | −27.237 | 27.606 | −91.187 | 1.00 | 34.85 | N |
| ATOM | 804 | C | LYS | | 117 | −31.858 | 23.321 | −93.290 | 1.00 | 21.29 | C |
| ATOM | 805 | O | LYS | | 117 | −32.096 | 23.522 | −94.481 | 1.00 | 20.12 | O |
| ATOM | 806 | N | ALA | | 118 | −32.550 | 22.470 | −92.537 | 1.00 | 21.37 | N |
| ATOM | 807 | CA | ALA | | 118 | −33.688 | 21.716 | −93.053 | 1.00 | 22.04 | C |
| ATOM | 808 | CB | ALA | | 118 | −33.491 | 20.227 | −92.800 | 1.00 | 22.43 | C |
| ATOM | 809 | C | ALA | | 118 | −34.944 | 22.203 | −92.337 | 1.00 | 21.60 | C |
| ATOM | 810 | O | ALA | | 118 | −35.854 | 21.423 | −92.060 | 1.00 | 20.00 | O |
| ATOM | 811 | N | ARG | | 119 | −34.971 | 23.496 | −92.028 | 1.00 | 22.99 | N |
| ATOM | 812 | CA | ARG | | 119 | −36.100 | 24.110 | −91.336 | 1.00 | 24.09 | C |
| ATOM | 813 | CB | ARG | | 119 | −37.348 | 24.102 | −92.228 | 1.00 | 26.58 | C |
| ATOM | 814 | CG | ARG | | 119 | −37.172 | 24.805 | −93.570 | 1.00 | 30.16 | C |
| ATOM | 815 | CD | ARG | | 119 | −38.498 | 24.876 | −94.337 | 1.00 | 33.21 | C |
| ATOM | 816 | NE | ARG | | 119 | −39.152 | 23.570 | −94.366 | 1.00 | 38.79 | N |
| ATOM | 817 | CZ | ARG | | 119 | −40.456 | 23.375 | −94.576 | 1.00 | 39.14 | C |
| ATOM | 818 | NH1 | ARG | | 119 | −41.273 | 24.405 | −94.784 | 1.00 | 36.88 | N |
| ATOM | 819 | NH2 | ARG | | 119 | −40.945 | 22.140 | −94.553 | 1.00 | 38.74 | N |
| ATOM | 820 | C | ARG | | 119 | −36.393 | 23.380 | −90.030 | 1.00 | 24.50 | C |
| ATOM | 821 | O | ARG | | 119 | −37.540 | 23.324 | −89.578 | 1.00 | 21.47 | O |
| ATOM | 822 | N | GLY | | 120 | −35.344 | 22.814 | −89.432 | 1.00 | 22.49 | N |
| ATOM | 823 | CA | GLY | | 120 | −35.491 | 22.116 | −88.168 | 1.00 | 22.55 | C |
| ATOM | 824 | C | GLY | | 120 | −35.872 | 20.652 | −88.236 | 1.00 | 22.32 | C |
| ATOM | 825 | O | GLY | | 120 | −35.942 | 19.989 | −87.202 | 1.00 | 22.89 | O |
| ATOM | 826 | N | ASP | | 121 | −36.106 | 20.136 | −89.439 | 1.00 | 21.83 | N |
| ATOM | 827 | CA | ASP | | 121 | −36.494 | 18.733 | −89.602 | 1.00 | 22.77 | C |
| ATOM | 828 | CB | ASP | | 121 | −36.934 | 18.478 | −91.052 | 1.00 | 22.05 | C |
| ATOM | 829 | CG | ASP | | 121 | −37.315 | 17.024 | −91.302 | 1.00 | 23.31 | C |
| ATOM | 830 | OD1 | ASP | | 121 | −38.017 | 16.435 | −90.455 | 1.00 | 22.45 | O |
| ATOM | 831 | OD2 | ASP | | 121 | −36.926 | 16.472 | −92.352 | 1.00 | 22.47 | O |
| ATOM | 832 | C | ASP | | 121 | −35.378 | 17.757 | −89.220 | 1.00 | 23.21 | C |
| ATOM | 833 | O | ASP | | 121 | −34.233 | 17.915 | −89.644 | 1.00 | 22.01 | O |
| ATOM | 834 | N | LEU | | 122 | −35.724 | 16.760 | −88.409 | 1.00 | 22.66 | N |
| ATOM | 835 | CA | LEU | | 122 | −34.775 | 15.733 | −87.976 | 1.00 | 25.01 | C |
| ATOM | 836 | CB | LEU | | 122 | −34.839 | 15.549 | −86.461 | 1.00 | 24.51 | C |
| ATOM | 837 | CG | LEU | | 122 | −34.212 | 16.655 | −85.618 | 1.00 | 25.76 | C |
| ATOM | 838 | CD1 | LEU | | 122 | −34.657 | 16.509 | −84.169 | 1.00 | 25.46 | C |
| ATOM | 839 | CD2 | LEU | | 122 | −32.700 | 16.585 | −85.739 | 1.00 | 25.27 | C |
| ATOM | 840 | C | LEU | | 122 | −35.100 | 14.404 | −88.638 | 1.00 | 25.23 | C |
| ATOM | 841 | O | LEU | | 122 | −34.369 | 13.430 | −88.477 | 1.00 | 25.59 | O |
| ATOM | 842 | N | GLY | | 123 | −36.200 | 14.371 | −89.384 | 1.00 | 25.89 | N |
| ATOM | 843 | CA | GLY | | 123 | −36.606 | 13.143 | −90.036 | 1.00 | 24.99 | C |
| ATOM | 844 | C | GLY | | 123 | −37.362 | 12.276 | −89.055 | 1.00 | 26.47 | C |
| ATOM | 845 | O | GLY | | 123 | −37.696 | 12.724 | −87.949 | 1.00 | 26.25 | O |
| ATOM | 846 | N | ALA | | 124 | −37.638 | 11.037 | −89.451 | 1.00 | 26.84 | N |
| ATOM | 847 | CA | ALA | | 124 | −38.363 | 10.102 | −88.599 | 1.00 | 29.44 | C |
| ATOM | 848 | CB | ALA | | 124 | −39.169 | 9.132 | −89.455 | 1.00 | 29.71 | C |
| ATOM | 849 | C | ALA | | 124 | −37.406 | 9.330 | −87.695 | 1.00 | 31.97 | C |
| ATOM | 850 | O | ALA | | 124 | −36.233 | 9.141 | −88.025 | 1.00 | 33.44 | O |
| ATOM | 851 | N | PHE | | 125 | −37.912 | 8.883 | −86.550 | 1.00 | 32.29 | N |
| ATOM | 852 | CA | PHE | | 125 | −37.099 | 8.130 | −85.604 | 1.00 | 32.91 | C |
| ATOM | 853 | CB | PHE | | 125 | −36.170 | 9.079 | −84.827 | 1.00 | 32.38 | C |
| ATOM | 854 | CG | PHE | | 125 | −36.889 | 10.195 | −84.090 | 1.00 | 33.81 | C |
| ATOM | 855 | CD1 | PHE | | 125 | −37.404 | 9.993 | −82.815 | 1.00 | 33.23 | C |
| ATOM | 856 | CD2 | PHE | | 125 | −37.029 | 11.453 | −84.671 | 1.00 | 33.83 | C |
| ATOM | 857 | CE1 | PHE | | 125 | −38.046 | 11.025 | −82.126 | 1.00 | 32.81 | C |
| ATOM | 858 | CE2 | PHE | | 125 | −37.671 | 12.493 | −83.991 | 1.00 | 33.48 | C |
| ATOM | 859 | CZ | PHE | | 125 | −38.179 | 12.277 | −82.713 | 1.00 | 33.57 | C |
| ATOM | 860 | C | PHE | | 125 | −37.965 | 7.324 | −84.651 | 1.00 | 33.85 | C |
| ATOM | 861 | O | PHE | | 125 | −39.153 | 7.607 | −84.482 | 1.00 | 33.96 | O |
| ATOM | 862 | N | SER | | 126 | −37.368 | 6.303 | −84.046 | 1.00 | 34.41 | N |
| ATOM | 863 | CA | SER | | 126 | −38.077 | 5.454 | −83.095 | 1.00 | 35.67 | C |
| ATOM | 864 | CB | SER | | 126 | −37.926 | 3.974 | −83.480 | 1.00 | 37.06 | C |
| ATOM | 865 | OG | SER | | 126 | −36.566 | 3.571 | −83.424 | 1.00 | 37.60 | O |
| ATOM | 866 | C | SER | | 126 | −37.480 | 5.689 | −81.712 | 1.00 | 34.93 | C |
| ATOM | 867 | O | SER | | 126 | −36.532 | 6.463 | −81.565 | 1.00 | 34.78 | O |
| ATOM | 868 | N | ARG | | 127 | −38.037 | 5.037 | −80.698 | 1.00 | 35.72 | N |
| ATOM | 869 | CA | ARG | | 127 | −37.516 | 5.196 | −79.349 | 1.00 | 37.20 | C |
| ATOM | 870 | CB | ARG | | 127 | −38.449 | 4.552 | −78.326 | 1.00 | 35.34 | C |
| ATOM | 871 | CG | ARG | | 127 | −39.688 | 5.376 | −78.027 | 1.00 | 36.16 | C |
| ATOM | 872 | CD | ARG | | 127 | −40.505 | 4.726 | −76.941 | 1.00 | 34.47 | C |
| ATOM | 873 | NE | ARG | | 127 | −41.695 | 5.494 | −76.598 | 1.00 | 33.99 | N |
| ATOM | 874 | CZ | ARG | | 127 | −41.699 | 6.602 | −75.863 | 1.00 | 30.76 | C |
| ATOM | 875 | NH1 | ARG | | 127 | −40.567 | 7.097 | −75.380 | 1.00 | 28.17 | N |
| ATOM | 876 | NH2 | ARG | | 127 | −42.852 | 7.207 | −75.600 | 1.00 | 30.41 | N |
| ATOM | 877 | C | ARG | | 127 | −36.131 | 4.572 | −79.247 | 1.00 | 39.06 | C |
| ATOM | 878 | O | ARG | | 127 | −35.862 | 3.531 | −79.849 | 1.00 | 39.54 | O |
| ATOM | 879 | N | GLY | | 128 | −35.252 | 5.226 | −78.497 | 1.00 | 39.85 | N |
| ATOM | 880 | CA | GLY | | 128 | −33.905 | 4.716 | −78.322 | 1.00 | 40.80 | C |
| ATOM | 881 | C | GLY | | 128 | −32.859 | 5.390 | −79.186 | 1.00 | 40.23 | C |
| ATOM | 882 | O | GLY | | 128 | −31.697 | 4.988 | −79.168 | 1.00 | 42.50 | O |
| ATOM | 883 | N | GLN | | 129 | −33.250 | 6.416 | −79.938 | 1.00 | 39.06 | N |
| ATOM | 884 | CA | GLN | | 129 | −32.304 | 7.108 | −80.805 | 1.00 | 37.01 | C |
| ATOM | 885 | CB | GLN | | 129 | −32.859 | 7.217 | −82.225 | 1.00 | 38.65 | C |
| ATOM | 886 | CG | GLN | | 129 | −33.359 | 5.909 | −82.820 | 1.00 | 40.19 | C |
| ATOM | 887 | CD | GLN | | 129 | −33.827 | 6.076 | −84.256 | 1.00 | 40.77 | C |
| ATOM | 888 | OE1 | GLN | | 129 | −34.557 | 5.239 | −84.788 | 1.00 | 42.20 | O |
| ATOM | 889 | NE2 | GLN | | 129 | −33.399 | 7.158 | −84.894 | 1.00 | 42.66 | N |
| ATOM | 890 | C | GLN | | 129 | −31.941 | 8.501 | −80.320 | 1.00 | 36.26 | C |
| ATOM | 891 | O | GLN | | 129 | −30.781 | 8.893 | −80.372 | 1.00 | 35.91 | O |
| ATOM | 892 | N | MET | | 130 | −32.932 | 9.253 | −79.854 | 1.00 | 34.08 | N |
| ATOM | 893 | CA | MET | | 130 | −32.691 | 10.617 | −79.392 | 1.00 | 33.69 | C |
| ATOM | 894 | CB | MET | | 130 | −33.859 | 11.521 | −79.805 | 1.00 | 35.18 | C |
| ATOM | 895 | CG | MET | | 130 | −34.250 | 11.427 | −81.272 | 1.00 | 35.18 | C |
| ATOM | 896 | SD | MET | | 130 | −33.007 | 12.146 | −82.338 | 1.00 | 37.15 | S |
| ATOM | 897 | CE | MET | | 130 | −33.806 | 12.029 | −83.934 | 1.00 | 36.44 | C |
| ATOM | 898 | C | MET | | 130 | −32.526 | 10.701 | −77.880 | 1.00 | 31.67 | C |
| ATOM | 899 | O | MET | | 130 | −32.866 | 9.772 | −77.153 | 1.00 | 32.91 | O |
| ATOM | 900 | N | GLN | | 131 | −31.991 | 11.823 | −77.411 | 1.00 | 31.48 | N |
| ATOM | 901 | CA | GLN | | 131 | −31.836 | 12.044 | −75.982 | 1.00 | 32.27 | C |
| ATOM | 902 | CB | GLN | | 131 | −31.345 | 13.467 | −75.734 | 1.00 | 32.97 | C |
| ATOM | 903 | CG | GLN | | 131 | −29.901 | 13.673 | −76.145 | 1.00 | 34.90 | C |
| ATOM | 904 | CD | GLN | | 131 | −29.493 | 15.128 | −76.147 | 1.00 | 36.75 | C |
| ATOM | 905 | OE1 | GLN | | 131 | −28.345 | 15.463 | −75.856 | 1.00 | 38.84 | O |
| ATOM | 906 | NE2 | GLN | | 131 | −30.430 | 16.004 | −76.496 | 1.00 | 36.93 | N |
| ATOM | 907 | C | GLN | | 131 | −33.218 | 11.830 | −75.366 | 1.00 | 32.66 | C |
| ATOM | 908 | O | GLN | | 131 | −34.225 | 12.343 | −75.875 | 1.00 | 32.82 | O |
| ATOM | 909 | N | LYS | | 132 | −33.259 | 11.067 | −74.279 | 1.00 | 30.69 | N |
| ATOM | 910 | CA | LYS | | 132 | −34.500 | 10.710 | −73.590 | 1.00 | 29.26 | C |
| ATOM | 911 | CB | LYS | | 132 | −34.156 | 10.100 | −72.227 | 1.00 | 28.95 | C |
| ATOM | 912 | CG | LYS | | 132 | −35.306 | 9.342 | −71.594 | 1.00 | 29.31 | C |
| ATOM | 913 | CD | LYS | | 132 | −35.843 | 8.279 | −72.536 | 1.00 | 26.37 | C |
| ATOM | 914 | CE | LYS | | 132 | −37.086 | 7.632 | −71.968 | 1.00 | 28.15 | C |
| ATOM | 915 | NZ | LYS | | 132 | −37.755 | 6.754 | −72.962 | 1.00 | 29.18 | N |
| ATOM | 916 | C | LYS | | 132 | −35.587 | 11.789 | −73.425 | 1.00 | 28.00 | C |
| ATOM | 917 | O | LYS | | 132 | −36.744 | 11.561 | −73.778 | 1.00 | 28.35 | O |
| ATOM | 918 | N | PRO | | 133 | −35.246 | 12.961 | −72.862 | 1.00 | 26.58 | N |
| ATOM | 919 | CD | PRO | | 133 | −33.969 | 13.407 | −72.286 | 1.00 | 26.85 | C |
| ATOM | 920 | CA | PRO | | 133 | −36.281 | 13.992 | −72.702 | 1.00 | 26.73 | C |
| ATOM | 921 | CB | PRO | | 133 | −35.506 | 15.168 | −72.113 | 1.00 | 26.29 | C |
| ATOM | 922 | CG | PRO | | 133 | −34.418 | 14.500 | −71.342 | 1.00 | 27.48 | C |
| ATOM | 923 | C | PRO | | 133 | −36.928 | 14.343 | −74.048 | 1.00 | 26.15 | C |
| ATOM | 924 | O | PRO | | 133 | −38.152 | 14.468 | −74.150 | 1.00 | 25.37 | O |
| ATOM | 925 | N | PHE | | 134 | −36.093 | 14.485 | −75.075 | 1.00 | 26.38 | N |
| ATOM | 926 | CA | PHE | | 134 | −36.562 | 14.819 | −76.420 | 1.00 | 25.94 | C |
| ATOM | 927 | CB | PHE | | 134 | −35.377 | 14.963 | −77.374 | 1.00 | 25.81 | C |
| ATOM | 928 | CG | PHE | | 134 | −35.738 | 15.568 | −78.708 | 1.00 | 23.50 | C |
| ATOM | 929 | CD1 | PHE | | 134 | −35.619 | 16.935 | −78.919 | 1.00 | 23.91 | C |
| ATOM | 930 | CD2 | PHE | | 134 | −36.204 | 14.773 | −79.744 | 1.00 | 24.67 | C |
| ATOM | 931 | CE1 | PHE | | 134 | −35.960 | 17.502 | −80.143 | 1.00 | 23.40 | C |
| ATOM | 932 | CE2 | PHE | | 134 | −36.549 | 15.331 | −80.967 | 1.00 | 23.00 | C |
| ATOM | 933 | CZ | PHE | | 134 | −36.425 | 16.700 | −81.166 | 1.00 | 21.42 | C |
| ATOM | 934 | C | PHE | | 134 | −37.481 | 13.723 | −76.939 | 1.00 | 25.70 | C |
| ATOM | 935 | O | PHE | | 134 | −38.558 | 13.998 | −77.472 | 1.00 | 26.34 | O |
| ATOM | 936 | N | GLU | | 135 | −37.045 | 12.477 | −76.778 | 1.00 | 24.98 | N |
| ATOM | 937 | CA | GLU | | 135 | −37.820 | 11.329 | −77.226 | 1.00 | 23.08 | C |
| ATOM | 938 | CB | GLU | | 135 | −37.062 | 10.038 | −76.922 | 1.00 | 24.77 | C |
| ATOM | 939 | CG | GLU | | 135 | −37.873 | 8.783 | −77.164 | 1.00 | 27.59 | C |
| ATOM | 940 | CD | GLU | | 135 | −37.165 | 7.538 | −76.665 | 1.00 | 29.43 | C |
| ATOM | 941 | OE1 | GLU | | 135 | −35.993 | 7.334 | −77.042 | 1.00 | 29.33 | O |
| ATOM | 942 | OE2 | GLU | | 135 | −37.782 | 6.767 | −75.898 | 1.00 | 30.32 | O |
| ATOM | 943 | C | GLU | | 135 | −39.204 | 11.242 | −76.591 | 1.00 | 23.38 | C |
| ATOM | 944 | O | GLU | | 135 | −40.212 | 11.102 | −77.291 | 1.00 | 22.48 | O |
| ATOM | 945 | N | ASP | | 136 | −39.255 | 11.299 | −75.265 | 1.00 | 22.85 | N |
| ATOM | 946 | CA | ASP | | 136 | −40.529 | 11.210 | −74.562 | 1.00 | 24.33 | C |
| ATOM | 947 | CB | ASP | | 136 | −40.310 | 11.246 | −73.046 | 1.00 | 26.28 | C |
| ATOM | 948 | CG | ASP | | 136 | −39.700 | 9.953 | −72.517 | 1.00 | 29.87 | C |
| ATOM | 949 | OD1 | ASP | | 136 | −40.062 | 8.873 | −73.035 | 1.00 | 29.17 | O |
| ATOM | 950 | OD2 | ASP | | 136 | −38.874 | 10.015 | −71.582 | 1.00 | 31.48 | O |
| ATOM | 951 | C | ASP | | 136 | −41.490 | 12.319 | −74.982 | 1.00 | 21.95 | C |
| ATOM | 952 | O | ASP | | 136 | −42.684 | 12.081 | −75.190 | 1.00 | 22.06 | O |
| ATOM | 953 | N | ALA | | 137 | −40.961 | 13.530 | −75.104 | 1.00 | 21.91 | N |
| ATOM | 954 | CA | ALA | | 137 | −41.768 | 14.666 | −75.522 | 1.00 | 21.26 | C |
| ATOM | 955 | CB | ALA | | 137 | −40.943 | 15.947 | −75.455 | 1.00 | 20.37 | C |
| ATOM | 956 | C | ALA | | 137 | −42.251 | 14.413 | −76.949 | 1.00 | 20.38 | C |
| ATOM | 957 | O | ALA | | 137 | −43.426 | 14.580 | −77.252 | 1.00 | 20.12 | O |
| ATOM | 958 | N | SER | | 138 | −41.341 | 13.981 | −77.824 | 1.00 | 22.62 | N |
| ATOM | 959 | CA | SER | | 138 | −41.697 | 13.720 | −79.219 | 1.00 | 22.67 | C |
| ATOM | 960 | CB | SER | | 138 | −40.473 | 13.249 | −80.012 | 1.00 | 20.70 | C |
| ATOM | 961 | OG | SER | | 138 | −39.488 | 14.264 | −80.071 | 1.00 | 19.37 | O |
| ATOM | 962 | C | SER | | 138 | −42.815 | 12.696 | −79.371 | 1.00 | 24.65 | C |
| ATOM | 963 | O | SER | | 138 | −43.728 | 12.889 | −80.173 | 1.00 | 25.67 | O |
| ATOM | 964 | N | PHE | | 139 | −42.746 | 11.608 | −78.610 | 1.00 | 25.28 | N |
| ATOM | 965 | CA | PHE | | 139 | −43.771 | 10.570 | −78.694 | 1.00 | 26.75 | C |
| ATOM | 966 | CB | PHE | | 139 | −43.216 | 9.221 | −78.208 | 1.00 | 28.02 | C |
| ATOM | 967 | CG | PHE | | 139 | −42.364 | 8.514 | −79.236 | 1.00 | 28.77 | C |
| ATOM | 968 | CD1 | PHE | | 139 | −41.032 | 8.865 | −79.418 | 1.00 | 29.76 | C |
| ATOM | 969 | CD2 | PHE | | 139 | −42.916 | 7.538 | −80.056 | 1.00 | 30.32 | C |
| ATOM | 970 | CE1 | PHE | | 139 | −40.260 | 8.253 | −80.411 | 1.00 | 29.94 | C |
| ATOM | 971 | CE2 | PHE | | 139 | −42.152 | 6.922 | −81.050 | 1.00 | 29.34 | C |
| ATOM | 972 | CZ | PHE | | 139 | −40.824 | 7.281 | −81.226 | 1.00 | 27.71 | C |
| ATOM | 973 | C | PHE | | 139 | −45.058 | 10.911 | −77.944 | 1.00 | 25.38 | C |
| ATOM | 974 | O | PHE | | 139 | −46.099 | 10.301 | −78.179 | 1.00 | 26.52 | O |
| ATOM | 975 | N | ALA | | 140 | −44.992 | 11.888 | −77.049 | 1.00 | 25.00 | N |
| ATOM | 976 | CA | ALA | | 140 | −46.172 | 12.291 | −76.296 | 1.00 | 25.17 | C |
| ATOM | 977 | CB | ALA | | 140 | −45.763 | 12.858 | −74.946 | 1.00 | 24.70 | C |
| ATOM | 978 | C | ALA | | 140 | −46.986 | 13.321 | −77.079 | 1.00 | 26.27 | C |
| ATOM | 979 | O | ALA | | 140 | −48.119 | 13.635 | −76.712 | 1.00 | 27.77 | O |
| ATOM | 980 | N | LEU | | 141 | −46.409 | 13.853 | −78.151 | 1.00 | 25.18 | N |
| ATOM | 981 | CA | LEU | | 141 | −47.111 | 14.834 | −78.976 | 1.00 | 25.68 | C |
| ATOM | 982 | CB | LEU | | 141 | −46.117 | 15.761 | −79.694 | 1.00 | 23.76 | C |
| ATOM | 983 | CG | LEU | | 141 | −45.236 | 16.762 | −78.937 | 1.00 | 22.59 | C |
| ATOM | 984 | CD1 | LEU | | 141 | −44.108 | 17.247 | −79.866 | 1.00 | 21.95 | C |
| ATOM | 985 | CD2 | LEU | | 141 | −46.082 | 17.939 | −78.455 | 1.00 | 23.55 | C |
| ATOM | 986 | C | LEU | | 141 | −47.912 | 14.098 | −80.037 | 1.00 | 26.34 | C |
| ATOM | 987 | O | LEU | | 141 | −47.556 | 12.988 | −80.428 | 1.00 | 27.81 | O |
| ATOM | 988 | N | ARG | | 142 | −49.008 | 14.693 | −80.492 | 1.00 | 26.50 | N |
| ATOM | 989 | CA | ARG | | 142 | −49.768 | 14.080 | −81.572 | 1.00 | 26.45 | C |
| ATOM | 990 | CB | ARG | | 142 | −51.279 | 14.200 | −81.354 | 1.00 | 29.70 | C |
| ATOM | 991 | CG | ARG | | 142 | −51.846 | 13.134 | −80.418 | 1.00 | 34.09 | C |
| ATOM | 992 | CD | ARG | | 142 | −51.898 | 13.621 | −78.985 | 1.00 | 37.77 | C |
| ATOM | 993 | NE | ARG | | 142 | −52.891 | 14.679 | −78.840 | 1.00 | 42.23 | N |
| ATOM | 994 | CZ | ARG | | 142 | −53.231 | 15.233 | −77.683 | 1.00 | 44.44 | C |
| ATOM | 995 | NH1 | ARG | | 142 | −52.658 | 14.825 | −76.557 | 1.00 | 48.32 | N |
| ATOM | 996 | NH2 | ARG | | 142 | −54.146 | 16.191 | −77.652 | 1.00 | 43.79 | N |
| ATOM | 997 | C | ARG | | 142 | −49.353 | 14.842 | −82.822 | 1.00 | 25.79 | C |
| ATOM | 998 | O | ARG | | 142 | −48.806 | 15.943 | −82.727 | 1.00 | 24.41 | O |
| ATOM | 999 | N | THR | | 143 | −49.581 | 14.261 | −83.990 | 1.00 | 25.14 | N |
| ATOM | 1000 | CA | THR | | 143 | −49.201 | 14.929 | −85.223 | 1.00 | 25.72 | C |
| ATOM | 1001 | CB | THR | | 143 | −49.670 | 14.131 | −86.440 | 1.00 | 25.95 | C |
| ATOM | 1002 | OG1 | THR | | 143 | −49.005 | 12.861 | −86.441 | 1.00 | 31.01 | O |
| ATOM | 1003 | CG2 | THR | | 143 | −49.345 | 14.871 | −87.725 | 1.00 | 25.05 | C |
| ATOM | 1004 | C | THR | | 143 | −49.794 | 16.331 | −85.270 | 1.00 | 25.02 | C |
| ATOM | 1005 | O | THR | | 143 | −50.991 | 16.522 | −85.034 | 1.00 | 24.82 | O |
| ATOM | 1006 | N | GLY | | 144 | −48.944 | 17.313 | −85.555 | 1.00 | 24.79 | N |
| ATOM | 1007 | CA | GLY | | 144 | −49.397 | 18.689 | −85.626 | 1.00 | 23.53 | C |
| ATOM | 1008 | C | GLY | | 144 | −49.163 | 19.506 | −84.368 | 1.00 | 23.60 | C |
| ATOM | 1009 | O | GLY | | 144 | −49.125 | 20.741 | −84.428 | 1.00 | 23.43 | O |
| ATOM | 1010 | N | GLU | | 145 | −49.001 | 18.838 | −83.227 | 1.00 | 21.53 | N |
| ATOM | 1011 | CA | GLU | | 145 | −48.788 | 19.545 | −81.961 | 1.00 | 21.14 | C |
| ATOM | 1012 | CB | GLU | | 145 | −49.223 | 18.681 | −80.776 | 1.00 | 22.83 | C |
| ATOM | 1013 | CG | GLU | | 145 | −50.663 | 18.208 | −80.823 | 1.00 | 25.99 | C |
| ATOM | 1014 | CD | GLU | | 145 | −51.059 | 17.490 | −79.550 | 1.00 | 26.70 | C |
| ATOM | 1015 | OE1 | GLU | | 145 | −50.337 | 16.548 | −79.151 | 1.00 | 28.91 | O |
| ATOM | 1016 | OE2 | GLU | | 145 | −52.084 | 17.865 | −78.948 | 1.00 | 27.26 | O |
| ATOM | 1017 | C | GLU | | 145 | −47.348 | 19.973 | −81.729 | 1.00 | 21.30 | C |
| ATOM | 1018 | O | GLU | | 145 | −46.410 | 19.390 | −82.282 | 1.00 | 21.08 | O |
| ATOM | 1019 | N | MET | | 146 | −47.190 | 20.989 | −80.889 | 1.00 | 18.68 | N |
| ATOM | 1020 | CA | MET | | 146 | −45.882 | 21.526 | −80.552 | 1.00 | 19.46 | C |
| ATOM | 1021 | CB | MET | | 146 | −45.783 | 22.981 | −81.009 | 1.00 | 18.85 | C |
| ATOM | 1022 | CG | MET | | 146 | −44.435 | 23.639 | −80.761 | 1.00 | 18.89 | C |
| ATOM | 1023 | SD | MET | | 146 | −44.413 | 25.338 | −81.381 | 1.00 | 21.55 | S |
| ATOM | 1024 | CE | MET | | 146 | −45.240 | 26.201 | −80.028 | 1.00 | 17.39 | C |
| ATOM | 1025 | C | MET | | 146 | −45.671 | 21.431 | −79.042 | 1.00 | 18.99 | C |
| ATOM | 1026 | O | MET | | 146 | −46.626 | 21.446 | −78.265 | 1.00 | 20.10 | O |
| ATOM | 1027 | N | SER | | 147 | −44.414 | 21.325 | −78.634 | 1.00 | 17.69 | N |
| ATOM | 1028 | CA | SER | | 147 | −44.077 | 21.231 | −77.220 | 1.00 | 17.26 | C |
| ATOM | 1029 | CB | SER | | 147 | −42.826 | 20.368 | −77.028 | 1.00 | 15.40 | C |
| ATOM | 1030 | OG | SER | | 147 | −41.666 | 21.097 | −77.441 | 1.00 | 15.70 | O |
| ATOM | 1031 | C | SER | | 147 | −43.771 | 22.605 | −76.642 | 1.00 | 16.73 | C |
| ATOM | 1032 | O | SER | | 147 | −43.735 | 23.606 | −77.357 | 1.00 | 17.85 | O |
| ATOM | 1033 | N | GLY | | 148 | −43.561 | 22.626 | −75.330 | 1.00 | 17.38 | N |
| ATOM | 1034 | CA | GLY | | 148 | −43.161 | 23.837 | −74.647 | 1.00 | 16.78 | C |
| ATOM | 1035 | C | GLY | | 148 | −41.661 | 23.636 | −74.513 | 1.00 | 20.67 | C |
| ATOM | 1036 | O | GLY | | 148 | −41.094 | 22.813 | −75.240 | 1.00 | 20.08 | O |
| ATOM | 1037 | N | PRO | | 149 | −40.973 | 24.353 | −73.617 | 1.00 | 20.90 | N |
| ATOM | 1038 | CD | PRO | | 149 | −41.402 | 25.484 | −72.777 | 1.00 | 22.14 | C |
| ATOM | 1039 | CA | PRO | | 149 | −39.525 | 24.135 | −73.508 | 1.00 | 22.59 | C |
| ATOM | 1040 | CB | PRO | | 149 | −39.089 | 25.180 | −72.480 | 1.00 | 22.61 | C |
| ATOM | 1041 | CG | PRO | | 149 | −40.119 | 26.280 | −72.651 | 1.00 | 23.58 | C |
| ATOM | 1042 | C | PRO | | 149 | −39.193 | 22.716 | −73.042 | 1.00 | 21.40 | C |
| ATOM | 1043 | O | PRO | | 149 | −39.727 | 22.253 | −72.041 | 1.00 | 21.18 | O |
| ATOM | 1044 | N | VAL | | 150 | −38.320 | 22.029 | −73.775 | 1.00 | 20.95 | N |
| ATOM | 1045 | CA | VAL | | 150 | −37.901 | 20.675 | −73.410 | 1.00 | 20.87 | C |
| ATOM | 1046 | CB | VAL | | 150 | −38.278 | 19.649 | −74.506 | 1.00 | 20.94 | C |
| ATOM | 1047 | CG1 | VAL | | 150 | −37.715 | 18.277 | −74.160 | 1.00 | 19.63 | C |
| ATOM | 1048 | CG2 | VAL | | 150 | −39.792 | 19.559 | −74.626 | 1.00 | 20.42 | C |
| ATOM | 1049 | C | VAL | | 150 | −36.379 | 20.694 | −73.199 | 1.00 | 22.53 | C |
| ATOM | 1050 | O | VAL | | 150 | −35.615 | 20.998 | −74.115 | 1.00 | 22.76 | O |
| ATOM | 1051 | N | PHE | | 151 | −35.950 | 20.371 | −71.979 | 1.00 | 22.62 | N |
| ATOM | 1052 | CA | PHE | | 151 | −34.538 | 20.398 | −71.622 | 1.00 | 24.13 | C |
| ATOM | 1053 | CB | PHE | | 151 | −34.390 | 20.800 | −70.147 | 1.00 | 23.82 | C |
| ATOM | 1054 | CG | PHE | | 151 | −35.044 | 22.116 | −69.803 | 1.00 | 25.62 | C |
| ATOM | 1055 | CD1 | PHE | | 151 | −36.420 | 22.212 | −69.674 | 1.00 | 24.72 | C |
| ATOM | 1056 | CD2 | PHE | | 151 | −34.277 | 23.257 | −69.621 | 1.00 | 25.61 | C |
| ATOM | 1057 | CE1 | PHE | | 151 | −37.019 | 23.422 | −69.373 | 1.00 | 25.22 | C |
| ATOM | 1058 | CE2 | PHE | | 151 | −34.870 | 24.475 | −69.319 | 1.00 | 24.74 | C |
| ATOM | 1059 | CZ | PHE | | 151 | −36.244 | 24.557 | −69.194 | 1.00 | 24.70 | C |
| ATOM | 1060 | C | PHE | | 151 | −33.768 | 19.090 | −71.852 | 1.00 | 24.55 | C |
| ATOM | 1061 | O | PHE | | 151 | −34.250 | 18.006 | −71.524 | 1.00 | 25.76 | O |
| ATOM | 1062 | N | THR | | 152 | −32.572 | 19.207 | −72.427 | 1.00 | 26.27 | N |
| ATOM | 1063 | CA | THR | | 152 | −31.693 | 18.057 | −72.663 | 1.00 | 24.71 | C |
| ATOM | 1064 | CB | THR | | 152 | −31.794 | 17.499 | −74.097 | 1.00 | 25.44 | C |
| ATOM | 1065 | OG1 | THR | | 152 | −31.284 | 18.472 | −75.015 | 1.00 | 25.05 | O |
| ATOM | 1066 | CG2 | THR | | 152 | −33.236 | 17.138 | −74.452 | 1.00 | 24.56 | C |
| ATOM | 1067 | C | THR | | 152 | −30.262 | 18.557 | −72.476 | 1.00 | 24.73 | C |
| ATOM | 1068 | O | THR | | 152 | −30.037 | 19.759 | −72.313 | 1.00 | 25.48 | O |
| ATOM | 1069 | N | ASP | | 153 | −29.299 | 17.641 | −72.516 | 1.00 | 27.83 | N |
| ATOM | 1070 | CA | ASP | | 153 | −27.890 | 17.999 | −72.358 | 1.00 | 28.92 | C |
| ATOM | 1071 | CB | ASP | | 153 | −27.038 | 16.736 | −72.294 | 1.00 | 32.71 | C |
| ATOM | 1072 | CG | ASP | | 153 | −26.902 | 16.203 | −70.885 | 1.00 | 35.95 | C |
| ATOM | 1073 | OD1 | ASP | | 153 | −27.773 | 16.504 | −70.040 | 1.00 | 37.21 | O |
| ATOM | 1074 | OD2 | ASP | | 153 | −25.923 | 15.474 | −70.620 | 1.00 | 38.87 | O |
| ATOM | 1075 | C | ASP | | 153 | −27.373 | 18.907 | −73.467 | 1.00 | 29.41 | C |
| ATOM | 1076 | O | ASP | | 153 | −26.347 | 19.574 | −73.304 | 1.00 | 30.52 | O |
| ATOM | 1077 | N | SER | | 154 | −28.080 | 18.929 | −74.592 | 1.00 | 27.80 | N |
| ATOM | 1078 | CA | SER | | 154 | −27.687 | 19.766 | −75.721 | 1.00 | 27.31 | C |
| ATOM | 1079 | CB | SER | | 154 | −28.281 | 19.222 | −77.015 | 1.00 | 28.73 | C |
| ATOM | 1080 | OG | SER | | 154 | −27.774 | 17.932 | −77.281 | 1.00 | 34.18 | O |
| ATOM | 1081 | C | SER | | 154 | −28.152 | 21.193 | −75.527 | 1.00 | 26.36 | C |
| ATOM | 1082 | O | SER | | 154 | −27.510 | 22.132 | −76.002 | 1.00 | 26.07 | O |
| ATOM | 1083 | N | GLY | | 155 | −29.273 | 21.347 | −74.830 | 1.00 | 25.85 | N |
| ATOM | 1084 | CA | GLY | | 155 | −29.826 | 22.668 | −74.584 | 1.00 | 23.67 | C |
| ATOM | 1085 | C | GLY | | 155 | −31.341 | 22.617 | −74.443 | 1.00 | 23.57 | C |
| ATOM | 1086 | O | GLY | | 155 | −31.903 | 21.589 | −74.050 | 1.00 | 22.45 | O |
| ATOM | 1087 | N | ILE | | 156 | −32.011 | 23.717 | −74.762 | 1.00 | 22.57 | N |
| ATOM | 1088 | CA | ILE | | 156 | −33.469 | 23.755 | −74.656 | 1.00 | 21.80 | C |
| ATOM | 1089 | CB | ILE | | 156 | −33.927 | 25.036 | −73.930 | 1.00 | 22.22 | C |
| ATOM | 1090 | CG2 | ILE | | 156 | −35.427 | 24.963 | −73.639 | 1.00 | 22.67 | C |
| ATOM | 1091 | CG1 | ILE | | 156 | −33.154 | 25.187 | −72.614 | 1.00 | 23.67 | C |
| ATOM | 1092 | CD1 | ILE | | 156 | −33.429 | 26.477 | −71.856 | 1.00 | 22.08 | C |
| ATOM | 1093 | C | ILE | | 156 | −34.111 | 23.690 | −76.051 | 1.00 | 21.35 | C |
| ATOM | 1094 | O | ILE | | 156 | −33.731 | 24.435 | −76.955 | 1.00 | 21.51 | O |
| ATOM | 1095 | N | HIS | | 157 | −35.085 | 22.801 | −76.219 | 1.00 | 20.45 | N |
| ATOM | 1096 | CA | HIS | | 157 | −35.750 | 22.639 | −77.510 | 1.00 | 20.71 | C |
| ATOM | 1097 | CB | HIS | | 157 | −35.720 | 21.181 | −77.973 | 1.00 | 21.57 | C |
| ATOM | 1098 | CG | HIS | | 157 | −34.393 | 20.505 | −77.850 | 1.00 | 26.02 | C |
| ATOM | 1099 | CD2 | HIS | | 157 | −33.715 | 20.057 | −76.766 | 1.00 | 25.70 | C |
| ATOM | 1100 | ND1 | HIS | | 157 | −33.657 | 20.117 | −78.949 | 1.00 | 24.87 | N |
| ATOM | 1101 | CE1 | HIS | | 157 | −32.587 | 19.456 | −78.549 | 1.00 | 28.61 | C |
| ATOM | 1102 | NE2 | HIS | | 157 | −32.597 | 19.403 | −77.227 | 1.00 | 27.09 | N |
| ATOM | 1103 | C | HIS | | 157 | −37.232 | 23.003 | −77.519 | 1.00 | 20.51 | C |
| ATOM | 1104 | O | HIS | | 157 | −37.899 | 23.004 | −76.483 | 1.00 | 18.94 | O |
| ATOM | 1105 | N | ILE | | 158 | −37.723 | 23.300 | −78.719 | 1.00 | 19.88 | N |
| ATOM | 1106 | CA | ILE | | 158 | −39.143 | 23.526 | −78.969 | 1.00 | 19.79 | C |
| ATOM | 1107 | CB | ILE | | 158 | −39.473 | 24.853 | −79.680 | 1.00 | 20.20 | C |
| ATOM | 1108 | CG2 | ILE | | 158 | −40.975 | 24.900 | −79.981 | 1.00 | 22.54 | C |
| ATOM | 1109 | CG1 | ILE | | 158 | −39.125 | 26.048 | −78.796 | 1.00 | 20.64 | C |
| ATOM | 1110 | CD1 | ILE | | 158 | −39.243 | 27.370 | −79.523 | 1.00 | 21.18 | C |
| ATOM | 1111 | C | ILE | | 158 | −39.294 | 22.407 | −79.993 | 1.00 | 19.18 | C |
| ATOM | 1112 | O | ILE | | 158 | −38.526 | 22.343 | −80.958 | 1.00 | 20.90 | O |
| ATOM | 1113 | N | ILE | | 159 | −40.248 | 21.513 | −79.781 | 1.00 | 19.24 | N |
| ATOM | 1114 | CA | ILE | | 159 | −40.432 | 20.394 | −80.693 | 1.00 | 19.82 | C |
| ATOM | 1115 | CB | ILE | | 159 | −40.296 | 19.057 | −79.933 | 1.00 | 20.20 | C |
| ATOM | 1116 | CG2 | ILE | | 159 | −40.390 | 17.892 | −80.900 | 1.00 | 19.48 | C |
| ATOM | 1117 | CG1 | ILE | | 159 | −38.978 | 19.040 | −79.151 | 1.00 | 21.22 | C |
| ATOM | 1118 | CD1 | ILE | | 159 | −38.818 | 17.835 | −78.225 | 1.00 | 20.27 | C |
| ATOM | 1119 | C | ILE | | 159 | −41.788 | 20.418 | −81.383 | 1.00 | 20.04 | C |
| ATOM | 1120 | O | ILE | | 159 | −42.813 | 20.655 | −80.748 | 1.00 | 20.28 | O |
| ATOM | 1121 | N | LEU | | 160 | −41.784 | 20.173 | −82.691 | 1.00 | 19.71 | N |
| ATOM | 1122 | CA | LEU | | 160 | −43.016 | 20.139 | −83.470 | 1.00 | 21.46 | C |
| ATOM | 1123 | CB | LEU | | 160 | −43.002 | 21.236 | −84.543 | 1.00 | 18.33 | C |
| ATOM | 1124 | CG | LEU | | 160 | −44.134 | 21.169 | −85.578 | 1.00 | 20.27 | C |
| ATOM | 1125 | CD1 | LEU | | 160 | −45.439 | 21.610 | −84.933 | 1.00 | 18.83 | C |
| ATOM | 1126 | CD2 | LEU | | 160 | −43.807 | 22.054 | −86.777 | 1.00 | 20.48 | C |
| ATOM | 1127 | C | LEU | | 160 | −43.126 | 18.776 | −84.144 | 1.00 | 19.87 | C |
| ATOM | 1128 | O | LEU | | 160 | −42.265 | 18.412 | −84.940 | 1.00 | 22.21 | O |
| ATOM | 1129 | N | ARG | | 161 | −44.164 | 18.013 | −83.814 | 1.00 | 20.44 | N |
| ATOM | 1130 | CA | ARG | | 161 | −44.331 | 16.707 | −84.444 | 1.00 | 21.77 | C |
| ATOM | 1131 | CB | ARG | | 161 | −45.111 | 15.730 | −83.545 | 1.00 | 20.86 | C |
| ATOM | 1132 | CG | ARG | | 161 | −45.326 | 14.386 | −84.234 | 1.00 | 22.18 | C |
| ATOM | 1133 | CD | ARG | | 161 | −46.023 | 13.337 | −83.369 | 1.00 | 24.94 | C |
| ATOM | 1134 | NE | ARG | | 161 | −46.353 | 12.173 | −84.194 | 1.00 | 26.23 | N |
| ATOM | 1135 | CZ | ARG | | 161 | −47.072 | 11.129 | −83.792 | 1.00 | 26.97 | C |
| ATOM | 1136 | NH1 | ARG | | 161 | −47.556 | 11.072 | −82.555 | 1.00 | 26.11 | N |
| ATOM | 1137 | NH2 | ARG | | 161 | −47.334 | 10.150 | −84.649 | 1.00 | 25.86 | N |
| ATOM | 1138 | C | ARG | | 161 | −45.058 | 16.885 | −85.771 | 1.00 | 21.34 | C |
| ATOM | 1139 | O | ARG | | 161 | −46.184 | 17.399 | −85.819 | 1.00 | 21.53 | O |
| ATOM | 1140 | N | THR | | 162 | −44.414 | 16.443 | −86.848 | 1.00 | 20.99 | N |
| ATOM | 1141 | CA | THR | | 162 | −44.973 | 16.587 | −88.183 | 1.00 | 21.74 | C |
| ATOM | 1142 | CB | THR | | 162 | −43.902 | 17.093 | −89.164 | 1.00 | 21.75 | C |
| ATOM | 1143 | OG1 | THR | | 162 | −42.786 | 16.193 | −89.159 | 1.00 | 22.79 | O |
| ATOM | 1144 | CG2 | THR | | 162 | −43.423 | 18.476 | −88.756 | 1.00 | 20.14 | C |
| ATOM | 1145 | C | THR | | 162 | −45.598 | 15.321 | −88.763 | 1.00 | 22.35 | C |
| ATOM | 1146 | O | THR | | 162 | −46.410 | 15.401 | −89.681 | 1.00 | 21.77 | O |
| ATOM | 1147 | N | GLU | | 163 | −45.219 | 14.160 | −88.240 | 1.00 | 23.01 | N |
| ATOM | 1148 | CA | GLU | | 163 | −45.764 | 12.892 | −88.722 | 1.00 | 24.66 | C |
| ATOM | 1149 | CB | GLU | | 163 | −44.922 | 12.351 | −89.886 | 1.00 | 26.75 | C |
| ATOM | 1150 | CG | GLU | | 163 | −45.067 | 13.113 | −91.199 | 1.00 | 29.47 | C |
| ATOM | 1151 | CD | GLU | | 163 | −44.221 | 12.520 | −92.322 | 1.00 | 31.32 | C |
| ATOM | 1152 | OE1 | GLU | | 163 | −43.978 | 11.295 | −92.310 | 1.00 | 34.13 | O |
| ATOM | 1153 | OE2 | GLU | | 163 | −43.810 | 13.275 | −93.229 | 1.00 | 33.14 | O |
| ATOM | 1154 | C | GLU | | 163 | −45.791 | 11.861 | −87.599 | 1.00 | 24.13 | C |
| ATOM | 1155 | O | GLU | | 163 | −45.014 | 12.024 | −86.636 | 1.00 | 23.91 | O |
| ATOM | 1156 | OXT | GLU | | 163 | −46.576 | 10.894 | −87.702 | 1.00 | 22.62 | O |
| TER | 1157 | | GLU | | 163 | | | | | | |
| ATOM | 1158 | OH2 | TIP | S | 1 | −30.337 | 14.954 | −72.530 | 1.00 | 33.70 | O |
| ATOM | 1159 | OH2 | TIP | S | 2 | −28.518 | 12.454 | −73.081 | 1.00 | 59.22 | O |
| ATOM | 1160 | OH2 | TIP | S | 3 | −42.407 | 18.833 | −67.228 | 1.00 | 25.30 | O |
| ATOM | 1161 | OH2 | TIP | S | 4 | −30.117 | 28.059 | −91.462 | 1.00 | 25.64 | O |
| ATOM | 1162 | OH2 | TIP | S | 5 | −40.380 | 17.343 | −89.509 | 1.00 | 19.75 | O |
| ATOM | 1163 | OH2 | TIP | S | 6 | −51.559 | 29.839 | −80.956 | 1.00 | 23.09 | O |
| ATOM | 1164 | OH2 | TIP | S | 7 | −33.084 | 23.028 | −85.769 | 1.00 | 18.45 | O |
| ATOM | 1165 | OH2 | TIP | S | 8 | −49.705 | 15.932 | −76.489 | 1.00 | 20.89 | O |
| ATOM | 1166 | OH2 | TIP | S | 9 | −48.270 | 17.998 | −75.530 | 1.00 | 23.27 | O |
| ATOM | 1167 | OH2 | TIP | S | 10 | −33.529 | 23.201 | −96.630 | 1.00 | 23.43 | O |
| ATOM | 1168 | OH2 | TIP | S | 11 | −39.504 | 21.647 | −90.269 | 1.00 | 27.54 | O |
| ATOM | 1169 | OH2 | TIP | S | 12 | −47.319 | 29.740 | −86.121 | 1.00 | 19.52 | O |
| ATOM | 1170 | OH2 | TIP | S | 13 | −31.282 | 32.273 | −80.133 | 1.00 | 34.78 | O |
| ATOM | 1171 | OH2 | TIP | S | 14 | −43.845 | 31.200 | −74.053 | 1.00 | 26.14 | O |
| ATOM | 1172 | OH2 | TIP | S | 15 | −35.552 | 8.241 | −79.720 | 1.00 | 29.46 | O |
| ATOM | 1173 | OH2 | TIP | S | 16 | −37.006 | 35.310 | −78.208 | 1.00 | 29.54 | O |
| ATOM | 1174 | OH2 | TIP | S | 17 | −33.961 | 25.655 | −93.710 | 1.00 | 26.17 | O |
| ATOM | 1175 | OH2 | TIP | S | 18 | −51.888 | 27.636 | −76.726 | 1.00 | 22.19 | O |
| ATOM | 1176 | OH2 | TIP | S | 19 | −26.271 | 30.912 | −80.966 | 1.00 | 28.70 | O |
| ATOM | 1177 | OH2 | TIP | S | 20 | −38.827 | 16.619 | −69.736 | 1.00 | 39.37 | O |
| ATOM | 1178 | OH2 | TIP | S | 21 | −49.434 | 28.867 | −71.190 | 1.00 | 20.55 | O |
| ATOM | 1179 | OH2 | TIP | S | 22 | −32.140 | 36.255 | −88.668 | 1.00 | 37.74 | O |
| ATOM | 1180 | OH2 | TIP | S | 23 | −60.015 | 26.342 | −70.598 | 1.00 | 21.35 | O |
| ATOM | 1181 | OH2 | TIP | S | 24 | −45.809 | 25.641 | −70.682 | 1.00 | 28.99 | O |
| ATOM | 1182 | OH2 | TIP | S | 25 | −42.897 | 28.468 | −72.565 | 1.00 | 43.74 | O |
| ATOM | 1183 | OH2 | TIP | S | 26 | −43.489 | 9.674 | −74.213 | 1.00 | 27.81 | O |
| ATOM | 1184 | OH2 | TIP | S | 27 | −50.822 | 30.594 | −64.230 | 1.00 | 40.56 | O |
| ATOM | 1185 | OH2 | TIP | S | 28 | −29.865 | 30.158 | −80.820 | 1.00 | 31.54 | O |
| ATOM | 1186 | OH2 | TIP | S | 29 | −33.525 | 35.890 | −81.494 | 1.00 | 34.70 | O |
| ATOM | 1187 | OH2 | TIP | S | 30 | −51.371 | 20.598 | −78.078 | 1.00 | 24.51 | O |
| ATOM | 1188 | OH2 | TIP | S | 31 | −45.449 | 8.539 | −87.591 | 1.00 | 43.02 | O |
| ATOM | 1189 | OH2 | TIP | S | 32 | −14.149 | 25.806 | −82.260 | 1.00 | 43.99 | O |
| ATOM | 1190 | OH2 | TIP | S | 33 | −41.517 | 14.015 | −67.503 | 1.00 | 55.85 | O |
| ATOM | 1191 | OH2 | TIP | S | 34 | −11.982 | 27.431 | −85.358 | 1.00 | 48.42 | O |
| ATOM | 1192 | OH2 | TIP | S | 35 | −40.187 | 3.296 | −81.256 | 1.00 | 45.12 | O |
| ATOM | 1193 | OH2 | TIP | S | 36 | −42.979 | 8.781 | −89.768 | 1.00 | 45.38 | O |
| ATOM | 1194 | OH2 | TIP | S | 37 | −27.607 | 33.252 | −86.887 | 1.00 | 45.41 | O |
| ATOM | 1195 | OH2 | TIP | S | 38 | −17.963 | 32.867 | −80.490 | 1.00 | 47.42 | O |
| ATOM | 1196 | OH2 | TIP | S | 39 | −28.697 | 35.008 | −66.626 | 1.00 | 48.87 | O |
| ATOM | 1197 | OH2 | TIP | S | 40 | −38.935 | 35.933 | −73.191 | 1.00 | 49.10 | O |
| ATOM | 1198 | OH2 | TIP | S | 41 | −50.114 | 12.504 | −75.199 | 1.00 | 33.28 | O |
| ATOM | 1199 | OH2 | TIP | S | 42 | −54.173 | 17.457 | −82.064 | 1.00 | 43.74 | O |
| ATOM | 1200 | OH2 | TIP | S | 43 | −32.369 | 36.085 | −73.534 | 1.00 | 41.64 | O |
| ATOM | 1201 | OH2 | TIP | S | 44 | −54.571 | 27.801 | −75.888 | 1.00 | 21.30 | O |
| ATOM | 1202 | OH2 | TIP | S | 45 | −41.306 | 18.099 | −92.015 | 1.00 | 24.32 | O |
| ATOM | 1203 | OH2 | TIP | S | 46 | −36.529 | 37.091 | −83.137 | 1.00 | 68.47 | O |
| ATOM | 1204 | OH2 | TIP | S | 47 | −49.709 | 22.115 | −80.069 | 1.00 | 21.44 | O |
| ATOM | 1205 | OH2 | TIP | S | 48 | −28.084 | 35.045 | −77.109 | 1.00 | 42.57 | O |
| ATOM | 1206 | OH2 | TIP | S | 49 | −42.317 | 33.269 | −90.557 | 1.00 | 46.57 | O |
| ATOM | 1207 | OH2 | TIP | S | 50 | −63.208 | 29.033 | −71.148 | 1.00 | 30.45 | O |
| ATOM | 1208 | OH2 | TIP | S | 51 | −62.437 | 27.647 | −66.494 | 1.00 | 64.69 | O |
| ATOM | 1209 | OH2 | TIP | S | 52 | −39.659 | 30.883 | −67.696 | 1.00 | 53.29 | O |
| ATOM | 1210 | OH2 | TIP | S | 53 | −35.857 | 17.335 | −94.700 | 1.00 | 43.07 | O |
| ATOM | 1211 | OH2 | TIP | S | 54 | −39.954 | 25.935 | −68.347 | 1.00 | 46.56 | O |
| ATOM | 1212 | OH2 | TIP | S | 55 | −43.716 | 19.857 | −92.127 | 1.00 | 42.42 | O |
| ATOM | 1213 | OH2 | TIP | S | 56 | −51.353 | 11.615 | −84.018 | 1.00 | 40.85 | O |
| ATOM | 1214 | OH2 | TIP | S | 57 | −30.888 | 11.817 | −70.131 | 1.00 | 58.48 | O |
| ATOM | 1215 | OH2 | TIP | S | 58 | −29.602 | 34.602 | −80.235 | 1.00 | 51.31 | O |
| ATOM | 1216 | OH2 | TIP | S | 59 | −48.994 | 10.803 | −79.387 | 1.00 | 43.69 | O |
| ATOM | 1217 | OH2 | TIP | S | 60 | −27.762 | 11.513 | −69.875 | 1.00 | 49.05 | O |
| ATOM | 1218 | OH2 | TIP | S | 61 | −39.270 | 12.603 | −69.751 | 1.00 | 58.22 | O |
| ATOM | 1219 | OH2 | TIP | S | 62 | −40.665 | 28.632 | −69.086 | 1.00 | 37.07 | O |
| ATOM | 1220 | OH2 | TIP | S | 63 | −54.886 | 30.276 | −77.021 | 1.00 | 41.33 | O |
| ATOM | 1221 | OH2 | TIP | S | 64 | −34.693 | 37.646 | −74.632 | 1.00 | 49.20 | O |
| ATOM | 1222 | OH2 | TIP | S | 65 | −48.301 | 39.542 | −79.955 | 1.00 | 45.44 | O |
| ATOM | 1223 | OH2 | TIP | S | 66 | −12.093 | 31.226 | −84.857 | 1.00 | 36.29 | O |
| ATOM | 1224 | OH2 | TIP | S | 67 | −51.607 | 32.331 | −70.705 | 1.00 | 27.61 | O |
| ATOM | 1225 | OH2 | TIP | S | 68 | −45.668 | 27.590 | −86.746 | 1.00 | 32.59 | O |
| ATOM | 1226 | OH2 | TIP | S | 69 | −48.636 | 23.216 | −83.337 | 1.00 | 22.73 | O |
| ATOM | 1227 | OH2 | TIP | S | 70 | −48.648 | 19.834 | −77.586 | 1.00 | 23.07 | O |
| ATOM | 1228 | OH2 | TIP | S | 71 | −33.514 | 7.561 | −75.757 | 1.00 | 35.63 | O |
| ATOM | 1229 | OH2 | TIP | S | 72 | −40.632 | 6.519 | −71.318 | 1.00 | 41.26 | O |
| ATOM | 1230 | OH2 | TIP | S | 73 | −62.269 | 22.614 | −70.554 | 1.00 | 44.71 | O |
| ATOM | 1231 | OH2 | TIP | S | 74 | −46.905 | 25.356 | −83.742 | 1.00 | 24.16 | O |
| ATOM | 1232 | OH2 | TIP | S | 75 | −64.076 | 27.283 | −69.350 | 1.00 | 38.89 | O |
| ATOM | 1233 | OH2 | TIP | S | 76 | −50.744 | 37.111 | −68.228 | 1.00 | 77.57 | O |
| ATOM | 1234 | OH2 | TIP | S | 77 | −46.528 | 9.140 | −74.024 | 1.00 | 44.32 | O |
| ATOM | 1235 | OH2 | TIP | S | 78 | −53.608 | 14.722 | −84.884 | 1.00 | 65.45 | O |
| ATOM | 1236 | OH2 | TIP | S | 79 | −35.783 | 17.086 | −69.371 | 1.00 | 29.05 | O |
| ATOM | 1237 | OH2 | TIP | S | 80 | −46.630 | 28.205 | −64.350 | 1.00 | 102.24 | O |
| ATOM | 1238 | OH2 | TIP | S | 81 | −30.431 | 10.412 | −73.191 | 1.00 | 46.43 | O |
| ATOM | 1239 | OH2 | TIP | S | 82 | −46.601 | 33.719 | −72.320 | 1.00 | 45.24 | O |
| ATOM | 1240 | OH2 | TIP | S | 83 | −24.405 | 33.010 | −76.648 | 1.00 | 39.62 | O |
| ATOM | 1241 | OH2 | TIP | S | 84 | −30.625 | 22.533 | −71.178 | 1.00 | 32.04 | O |
| ATOM | 1242 | OH2 | TIP | S | 85 | −60.609 | 28.704 | −71.946 | 1.00 | 30.44 | O |
| ATOM | 1243 | OH2 | TIP | S | 86 | −32.078 | 6.382 | −73.765 | 1.00 | 34.41 | O |
| ATOM | 1244 | OH2 | TIP | S | 87 | −34.251 | 10.667 | −88.680 | 1.00 | 41.45 | O |
| ATOM | 1245 | OH2 | TIP | S | 88 | −36.267 | 26.858 | −98.249 | 1.00 | 34.75 | O |
| ATOM | 1246 | OH2 | TIP | S | 89 | −45.789 | 30.297 | −70.068 | 1.00 | 71.72 | O |
| ATOM | 1247 | OH2 | TIP | S | 90 | −49.953 | 9.533 | −82.267 | 1.00 | 52.83 | O |
| ATOM | 1248 | OH2 | TIP | S | 91 | −51.226 | 25.924 | −79.649 | 1.00 | 58.17 | O |
| ATOM | 1249 | OH2 | TIP | S | 92 | −19.141 | 34.369 | −78.416 | 1.00 | 61.29 | O |
| ATOM | 1250 | OH2 | TIP | S | 93 | −35.480 | 32.681 | −68.080 | 1.00 | 47.59 | O |
| ATOM | 1251 | OH2 | TIP | S | 94 | −25.051 | 34.480 | −83.347 | 1.00 | 80.24 | O |
| ATOM | 1252 | OH2 | TIP | S | 95 | −42.054 | 21.988 | −91.705 | 1.00 | 68.86 | O |
| ATOM | 1253 | OH2 | TIP | S | 96 | −46.250 | 9.131 | −92.563 | 1.00 | 56.89 | O |
| ATOM | 1254 | OH2 | TIP | S | 97 | −47.135 | 25.273 | −65.710 | 1.00 | 63.26 | O |
| ATOM | 1255 | OH2 | TIP | S | 98 | −30.311 | 34.474 | −88.303 | 1.00 | 57.79 | O |
| ATOM | 1256 | OH2 | TIP | S | 99 | −35.355 | 25.072 | −65.170 | 1.00 | 59.44 | O |
| ATOM | 1257 | OH2 | TIP | S | 100 | −36.754 | 37.084 | −87.208 | 1.00 | 43.03 | O |
| ATOM | 1258 | OH2 | TIP | S | 101 | −45.923 | 38.561 | −89.332 | 1.00 | 66.40 | O |
| ATOM | 1259 | OH2 | TIP | S | 102 | −15.689 | 32.401 | −84.562 | 1.00 | 39.71 | O |
| ATOM | 1260 | OH2 | TIP | S | 103 | −43.773 | 16.583 | −93.009 | 1.00 | 51.40 | O |
| ATOM | 1261 | OH2 | TIP | S | 104 | −43.338 | 39.639 | −84.060 | 1.00 | 56.17 | O |
| ATOM | 1262 | OH2 | TIP | S | 105 | −24.135 | 19.481 | −76.887 | 1.00 | 39.93 | O |
| ATOM | 1263 | OH2 | TIP | S | 106 | −47.385 | 31.671 | −88.352 | 1.00 | 40.93 | O |
| ATOM | 1264 | OH2 | TIP | S | 107 | −48.459 | 14.492 | −91.751 | 1.00 | 68.59 | O |
| ATOM | 1265 | OH2 | TIP | S | 108 | −31.614 | 25.550 | −62.406 | 1.00 | 54.75 | O |
| ATOM | 1266 | OH2 | TIP | S | 109 | −35.343 | 31.207 | −65.533 | 1.00 | 56.29 | O |
| ATOM | 1267 | OH2 | TIP | S | 110 | −55.480 | 23.643 | −79.605 | 1.00 | 45.22 | O |
| ATOM | 1268 | OH2 | TIP | S | 111 | −44.623 | 5.552 | −72.549 | 1.00 | 61.00 | O |
| ATOM | 1269 | OH2 | TIP | S | 112 | −48.101 | 9.551 | −68.190 | 1.00 | 54.21 | O |
| ATOM | 1270 | OH2 | TIP | S | 113 | −56.246 | 33.460 | −94.882 | 1.00 | 81.49 | O |
| ATOM | 1271 | OH2 | TIP | S | 114 | −43.104 | 37.261 | −90.400 | 1.00 | 63.81 | O |
| ATOM | 1272 | OH2 | TIP | S | 115 | −37.112 | 36.813 | −76.082 | 1.00 | 48.92 | O |
| ATOM | 1273 | OH2 | TIP | S | 116 | −42.414 | 31.371 | −93.110 | 1.00 | 49.45 | O |
| ATOM | 1274 | OH2 | TIP | S | 117 | −50.606 | 10.915 | −87.418 | 1.00 | 51.38 | O |
| TER | 1275 | | TIP | S | 117 | | | | | | |
| ATOM | 1276 | O1 | PGB | C | 180 | −48.146 | 28.969 | −68.081 | 1.00 | 38.62 | O |
| ATOM | 1277 | C2 | PGB | C | 180 | −49.219 | 28.811 | −67.252 | 1.00 | 38.72 | C |
| ATOM | 1278 | C3 | PGB | C | 180 | −50.409 | 29.694 | −67.685 | 1.00 | 35.22 | C |
| ATOM | 1279 | O4 | PGB | C | 180 | −51.001 | 29.188 | −68.770 | 1.00 | 33.83 | O |
| ATOM | 1280 | C5 | PGB | C | 180 | −52.287 | 29.537 | −68.878 | 1.00 | 28.44 | C |
| ATOM | 1281 | C6 | PGB | C | 180 | −52.870 | 28.902 | −70.156 | 1.00 | 26.59 | C |
| ATOM | 1282 | O7 | PGB | C | 180 | −52.380 | 29.548 | −71.242 | 1.00 | 26.26 | O |
| ATOM | 1283 | C8 | PGB | C | 180 | −52.834 | 29.033 | −72.401 | 1.00 | 22.02 | C |
| ATOM | 1284 | C9 | PGB | C | 180 | −52.235 | 29.817 | −73.588 | 1.00 | 22.80 | C |
| ATOM | 1285 | O10 | PGB | C | 180 | −50.918 | 30.089 | −73.362 | 1.00 | 21.70 | O |
| ATOM | 1286 | C11 | PGB | C | 180 | −50.361 | 30.778 | −74.376 | 1.00 | 21.92 | C |
| ATOM | 1287 | C12 | PGB | C | 180 | −48.830 | 30.876 | −74.161 | 1.00 | 23.81 | C |
| ATOM | 1288 | O13 | PGB | C | 180 | −48.261 | 29.652 | −74.181 | 1.00 | 22.16 | O |
| ATOM | 1289 | C14 | PGB | C | 180 | −46.990 | 29.737 | −73.745 | 1.00 | 24.89 | C |
| ATOM | 1290 | C15 | PGB | C | 180 | −46.390 | 28.323 | −73.475 | 1.00 | 22.50 | C |
| ATOM | 1291 | O16 | PGB | C | 180 | −46.615 | 27.475 | −74.497 | 1.00 | 24.49 | O |
| ATOM | 1292 | C17 | PGB | C | 180 | −46.095 | 26.316 | −74.209 | 1.00 | 25.81 | C |
| ATOM | 1293 | C18 | PGB | C | 180 | −46.306 | 25.292 | −75.326 | 1.00 | 26.85 | C |
| ATOM | 1294 | O19 | PGB | C | 180 | −47.601 | 25.057 | −75.416 | 1.00 | 27.15 | O |
| ATOM | 1295 | C20 | PGB | C | 180 | −47.841 | 24.476 | −76.556 | 1.00 | 28.30 | C |
| ATOM | 1296 | C21 | PGB | C | 180 | −49.043 | 23.932 | −77.320 | 1.00 | 31.36 | C |
| ATOM | 1297 | O22 | PGB | C | 180 | −49.871 | 24.934 | −77.537 | 1.00 | 32.87 | O |
| TER | 1298 | | PGB | C | 180 | | | | | | |
| ATOM | 1299 | C2 | NMP | H | 1 | −30.808 | 14.296 | −80.316 | 1.00 | 70.27 | C |
| ATOM | 1300 | O6 | NMP | H | 1 | −30.948 | 13.376 | −79.515 | 1.00 | 69.64 | O |
| ATOM | 1301 | C3 | NMP | H | 1 | −29.808 | 14.273 | −81.430 | 1.00 | 70.13 | C |
| ATOM | 1302 | C4 | NMP | H | 1 | −30.400 | 15.360 | −82.370 | 1.00 | 69.97 | C |
| ATOM | 1303 | C5 | NMP | H | 1 | −31.077 | 16.362 | −81.413 | 1.00 | 69.70 | C |
| ATOM | 1304 | N1 | NMP | H | 1 | −31.481 | 15.465 | −80.348 | 1.00 | 69.83 | N |
| ATOM | 1305 | C7 | NMP | H | 1 | −32.511 | 15.754 | −79.376 | 1.00 | 69.79 | C |
| TER | 1306 | | NMP | H | 1 | | | | | | |
| ATOM | 1307 | N2 | 221 | I | 1 | −26.221 | 19.961 | −84.419 | 1.00 | 80.03 | N |
| ATOM | 1308 | C3 | 221 | I | 1 | −24.903 | 20.102 | −85.039 | 1.00 | 82.20 | C |
| ATOM | 1309 | O8 | 221 | I | 1 | −24.741 | 21.070 | −85.948 | 1.00 | 81.64 | O |
| ATOM | 1310 | C4 | 221 | I | 1 | −23.904 | 19.251 | −84.658 | 1.00 | 83.96 | C |
| ATOM | 1311 | C17 | 221 | I | 1 | −22.506 | 19.234 | −85.155 | 1.00 | 89.77 | C |
| ATOM | 1312 | C18 | 221 | I | 1 | −21.648 | 20.413 | −85.186 | 1.00 | 95.63 | C |
| ATOM | 1313 | C22 | 221 | I | 1 | −21.106 | 20.890 | −86.305 | 1.00 | 97.93 | C |
| ATOM | 1314 | C21 | 221 | I | 1 | −20.354 | 22.053 | −85.843 | 1.00 | 101.00 | C |
| ATOM | 1315 | C20 | 221 | I | 1 | −20.586 | 22.056 | −84.530 | 1.00 | 101.71 | C |
| ATOM | 1316 | I | 221 | I | 1 | −19.761 | 23.513 | −83.285 | 1.00 | 111.65 | I |
| ATOM | 1317 | O19 | 221 | I | 1 | −21.374 | 21.084 | −84.055 | 1.00 | 99.20 | O |
| ATOM | 1318 | C5 | 221 | I | 1 | −24.281 | 18.247 | −83.627 | 1.00 | 81.88 | C |
| ATOM | 1319 | O9 | 221 | I | 1 | −23.400 | 17.453 | −83.265 | 1.00 | 81.59 | O |
| ATOM | 1320 | N6 | 221 | I | 1 | −25.534 | 18.204 | −83.133 | 1.00 | 79.46 | N |
| ATOM | 1321 | C10 | 221 | I | 1 | −25.837 | 17.259 | −82.192 | 1.00 | 77.42 | C |
| ATOM | 1322 | C11 | 221 | I | 1 | −25.909 | 15.878 | −82.578 | 1.00 | 76.11 | C |
| ATOM | 1323 | C12 | 221 | I | 1 | −26.225 | 14.902 | −81.613 | 1.00 | 75.54 | C |
| ATOM | 1324 | C13 | 221 | I | 1 | −26.482 | 15.306 | −80.266 | 1.00 | 75.42 | C |
| ATOM | 1325 | CL16 | 221 | I | 1 | −26.879 | 14.099 | −79.069 | 1.00 | 73.86 | CL |
| ATOM | 1326 | C14 | 221 | I | 1 | −26.413 | 16.674 | −79.875 | 1.00 | 75.47 | C |
| ATOM | 1327 | C15 | 221 | I | 1 | −26.094 | 17.655 | −80.834 | 1.00 | 76.29 | C |
| ATOM | 1328 | C1 | 221 | I | 1 | −26.472 | 19.069 | −83.530 | 1.00 | 79.63 | C |
| ATOM | 1329 | S7 | 221 | I | 1 | −28.129 | 18.952 | −82.810 | 1.00 | 77.87 | S |

Pin1 R14A/Pin-051 Interactions

The crystal structure of the Pin1/Pin-051 co-complex (the coordinate set is set forth in Table 5), was examined for the interactions made between the polypeptide, the Pin-051 inhibitor molecule, and the solvent. The phenyl ring of Pin-051 is positioned in the hydrophobic pocket, and has favorable van der Waals distances with side chains Met130, Phe134, Leu122, His59, and His157. The five-atom ring is positioned through the cysteine/serine valley. The two rings have a torsional angle of 75.2 degrees. One of the oxygen atoms (012) of the pyrazole ring is positioned to interact with Pin1 through a bridging water molecule (S5) to Arg69. Water molecules (S1-S10) located within the active site also define the interaction template.

TABLE 5
Set of atomic coordinates of Pin1 R14A with PIN-051 bound in the active site.

| | | | | | | | | | | | |
|---|---|---|---|---|---|---|---|---|---|---|---|
| ATOM | 1 | CB | LEU | | 7 | 21.710 | 45.910 | −29.510 | 1.00 | 51.04 | C |
| ATOM | 2 | CG | LEU | | 7 | 22.655 | 44.896 | −28.855 | 1.00 | 50.86 | C |
| ATOM | 3 | CD1 | LEU | | 7 | 21.892 | 44.048 | −27.852 | 1.00 | 50.76 | C |
| ATOM | 4 | CD2 | LEU | | 7 | 23.284 | 44.019 | −29.924 | 1.00 | 50.57 | C |
| ATOM | 5 | C | LEU | | 7 | 21.202 | 47.800 | −31.062 | 1.00 | 49.65 | C |
| ATOM | 6 | O | LEU | | 7 | 20.678 | 48.747 | −30.474 | 1.00 | 49.15 | O |
| ATOM | 7 | N | LEU | | 7 | 23.211 | 47.879 | −29.600 | 1.00 | 50.70 | N |
| ATOM | 8 | CA | LEU | | 7 | 22.321 | 46.995 | −30.405 | 1.00 | 49.99 | C |
| ATOM | 9 | N | PRO | | 8 | 20.821 | 47.431 | −32.295 | 1.00 | 48.93 | N |
| ATOM | 10 | CD | PRO | | 8 | 21.404 | 46.364 | −33.126 | 1.00 | 49.65 | C |
| ATOM | 11 | CA | PRO | | 8 | 19.753 | 48.128 | −33.018 | 1.00 | 48.61 | C |
| ATOM | 12 | CB | PRO | | 8 | 19.567 | 47.268 | −34.264 | 1.00 | 49.25 | C |
| ATOM | 13 | CG | PRO | | 8 | 20.949 | 46.754 | −34.515 | 1.00 | 49.65 | C |
| ATOM | 14 | C | PRO | | 8 | 18.476 | 48.222 | −32.174 | 1.00 | 47.77 | C |
| ATOM | 15 | O | PRO | | 8 | 18.343 | 47.545 | −31.151 | 1.00 | 48.38 | O |
| ATOM | 16 | N | PRO | | 9 | 17.516 | 49.059 | −32.602 | 1.00 | 45.88 | N |
| ATOM | 17 | CD | PRO | | 9 | 17.581 | 49.907 | −33.806 | 1.00 | 44.79 | C |
| ATOM | 18 | CA | PRO | | 9 | 16.244 | 49.261 | −31.898 | 1.00 | 43.38 | C |
| ATOM | 19 | CB | PRO | | 9 | 15.438 | 50.097 | −32.889 | 1.00 | 43.53 | C |
| ATOM | 20 | CG | PRO | | 9 | 16.493 | 50.925 | −33.539 | 1.00 | 44.60 | C |
| ATOM | 21 | C | PRO | | 9 | 15.484 | 48.012 | −31.452 | 1.00 | 41.11 | C |
| ATOM | 22 | O | PRO | | 9 | 14.982 | 47.243 | −32.280 | 1.00 | 39.96 | O |
| ATOM | 23 | N | GLY | | 10 | 15.402 | 47.825 | −30.136 | 1.00 | 38.89 | N |
| ATOM | 24 | CA | GLY | | 10 | 14.669 | 46.701 | −29.581 | 1.00 | 34.84 | C |
| ATOM | 25 | C | GLY | | 10 | 15.407 | 45.399 | −29.323 | 1.00 | 31.78 | C |
| ATOM | 26 | O | GLY | | 10 | 14.783 | 44.413 | −28.938 | 1.00 | 27.63 | O |
| ATOM | 27 | N | TRP | | 11 | 16.722 | 45.380 | −29.519 | 1.00 | 31.53 | N |
| ATOM | 28 | CA | TRP | | 11 | 17.494 | 44.157 | −29.302 | 1.00 | 30.85 | C |
| ATOM | 29 | CB | TRP | | 11 | 18.644 | 44.063 | −30.306 | 1.00 | 29.22 | C |
| ATOM | 30 | CG | TRP | | 11 | 18.200 | 43.807 | −31.703 | 1.00 | 27.38 | C |
| ATOM | 31 | CD2 | TRP | | 11 | 17.840 | 42.542 | −32.267 | 1.00 | 26.92 | C |
| ATOM | 32 | CE2 | TRP | | 11 | 17.458 | 42.775 | −33.607 | 1.00 | 26.20 | C |
| ATOM | 33 | CE3 | TRP | | 11 | 17.802 | 41.231 | −31.770 | 1.00 | 23.61 | C |
| ATOM | 34 | CD1 | TRP | | 11 | 18.028 | 44.730 | −32.694 | 1.00 | 26.89 | C |
| ATOM | 35 | NE1 | TRP | | 11 | 17.582 | 44.120 | −33.839 | 1.00 | 26.95 | N |
| ATOM | 36 | CZ2 | TRP | | 11 | 17.042 | 41.748 | −34.458 | 1.00 | 25.56 | C |
| ATOM | 37 | CZ3 | TRP | | 11 | 17.388 | 40.210 | −32.616 | 1.00 | 23.04 | C |
| ATOM | 38 | CH2 | TRP | | 11 | 17.013 | 40.475 | −33.947 | 1.00 | 25.62 | C |
| ATOM | 39 | C | TRP | | 11 | 18.057 | 44.015 | −27.897 | 1.00 | 31.36 | C |
| ATOM | 40 | O | TRP | | 11 | 18.389 | 45.003 | −27.241 | 1.00 | 31.53 | O |
| ATOM | 41 | N | GLU | | 12 | 18.155 | 42.770 | −27.437 | 1.00 | 30.10 | N |
| ATOM | 42 | CA | GLU | | 12 | 18.702 | 42.473 | −26.119 | 1.00 | 29.25 | C |
| ATOM | 43 | CB | GLU | | 12 | 17.583 | 42.356 | −25.071 | 1.00 | 30.87 | C |
| ATOM | 44 | CG | GLU | | 12 | 16.781 | 41.058 | −25.106 | 1.00 | 33.77 | C |
| ATOM | 45 | CD | GLU | | 12 | 15.676 | 41.025 | −24.057 | 1.00 | 36.32 | C |
| ATOM | 46 | OE1 | GLU | | 12 | 14.691 | 41.785 | −24.193 | 1.00 | 36.80 | O |
| ATOM | 47 | OE2 | GLU | | 12 | 15.792 | 40.241 | −23.091 | 1.00 | 37.75 | O |
| ATOM | 48 | C | GLU | | 12 | 19.479 | 41.162 | −26.202 | 1.00 | 28.29 | C |
| ATOM | 49 | O | GLU | | 12 | 19.225 | 40.336 | −27.080 | 1.00 | 25.04 | O |
| ATOM | 50 | N | LYS | | 13 | 20.434 | 40.984 | −25.296 | 1.00 | 28.86 | N |
| ATOM | 51 | CA | LYS | | 13 | 21.243 | 39.771 | −25.268 | 1.00 | 29.81 | C |
| ATOM | 52 | CB | LYS | | 13 | 22.611 | 40.060 | −24.639 | 1.00 | 32.70 | C |
| ATOM | 53 | CG | LYS | | 13 | 23.545 | 38.854 | −24.594 | 1.00 | 36.69 | C |
| ATOM | 54 | CD | LYS | | 13 | 24.775 | 39.124 | −23.733 | 1.00 | 40.30 | C |
| ATOM | 55 | CE | LYS | | 13 | 25.645 | 40.227 | −24.320 | 1.00 | 41.72 | C |
| ATOM | 56 | NZ | LYS | | 13 | 26.800 | 40.548 | −23.429 | 1.00 | 44.20 | N |
| ATOM | 57 | C | LYS | | 13 | 20.519 | 38.712 | −24.454 | 1.00 | 28.54 | C |
| ATOM | 58 | O | LYS | | 13 | 19.792 | 39.030 | −23.513 | 1.00 | 28.62 | O |
| ATOM | 59 | N | ALA | | 14 | 20.714 | 37.451 | −24.821 | 1.00 | 26.70 | N |
| ATOM | 60 | CA | ALA | | 14 | 20.082 | 36.344 | −24.114 | 1.00 | 26.09 | C |
| ATOM | 61 | CB | ALA | | 14 | 18.723 | 36.051 | −24.723 | 1.00 | 26.43 | C |
| ATOM | 62 | C | ALA | | 14 | 20.975 | 35.111 | −24.207 | 1.00 | 26.33 | C |
| ATOM | 63 | O | ALA | | 14 | 22.008 | 35.143 | −24.871 | 1.00 | 25.06 | O |
| ATOM | 64 | N | MET | | 15 | 20.575 | 34.034 | −23.536 | 1.00 | 28.75 | N |
| ATOM | 65 | CA | MET | | 15 | 21.326 | 32.780 | −23.559 | 1.00 | 30.00 | C |
| ATOM | 66 | CB | MET | | 15 | 22.044 | 32.529 | −22.230 | 1.00 | 35.03 | C |
| ATOM | 67 | CG | MET | | 15 | 23.160 | 33.489 | −21.886 | 1.00 | 40.51 | C |
| ATOM | 68 | SD | MET | | 15 | 24.120 | 32.838 | −20.486 | 1.00 | 46.91 | S |
| ATOM | 69 | CE | MET | | 15 | 22.932 | 32.996 | −19.157 | 1.00 | 44.15 | C |
| ATOM | 70 | C | MET | | 15 | 20.375 | 31.623 | −23.794 | 1.00 | 29.47 | C |
| ATOM | 71 | O | MET | | 15 | 19.312 | 31.555 | −23.179 | 1.00 | 28.00 | O |
| ATOM | 72 | N | SER | | 16 | 20.760 | 30.707 | −24.675 | 1.00 | 28.02 | N |
| ATOM | 73 | CA | SER | | 16 | 19.933 | 29.545 | −24.955 | 1.00 | 28.13 | C |
| ATOM | 74 | CB | SER | | 16 | 20.566 | 28.693 | −26.053 | 1.00 | 28.39 | C |
| ATOM | 75 | OG | SER | | 16 | 19.857 | 27.475 | −26.210 | 1.00 | 29.48 | O |
| ATOM | 76 | C | SER | | 16 | 19.787 | 28.706 | −23.689 | 1.00 | 29.27 | C |
| ATOM | 77 | O | SER | | 16 | 20.773 | 28.429 | −23.004 | 1.00 | 28.57 | O |
| ATOM | 78 | N | ARG | | 17 | 18.557 | 28.314 | −23.375 | 1.00 | 31.08 | N |
| ATOM | 79 | CA | ARG | | 17 | 18.301 | 27.491 | −22.199 | 1.00 | 33.36 | C |
| ATOM | 80 | CB | ARG | | 17 | 16.800 | 27.407 | −21.916 | 1.00 | 34.58 | C |
| ATOM | 81 | CG | ARG | | 17 | 16.208 | 28.683 | −21.347 | 1.00 | 36.05 | C |
| ATOM | 82 | CD | ARG | | 17 | 16.819 | 29.014 | −19.997 | 1.00 | 35.82 | C |
| ATOM | 83 | NE | ARG | | 17 | 16.458 | 30.358 | −19.559 | 1.00 | 37.11 | N |
| ATOM | 84 | CZ | ARG | | 17 | 15.246 | 30.719 | −19.149 | 1.00 | 37.23 | C |
| ATOM | 85 | NH1 | ARG | | 17 | 14.257 | 29.836 | −19.106 | 1.00 | 37.37 | N |
| ATOM | 86 | NH2 | ARG | | 17 | 15.022 | 31.976 | −18.799 | 1.00 | 38.19 | N |
| ATOM | 87 | C | ARG | | 17 | 18.848 | 26.092 | −22.438 | 1.00 | 34.32 | C |
| ATOM | 88 | O | ARG | | 17 | 19.125 | 25.351 | −21.498 | 1.00 | 33.68 | O |
| ATOM | 89 | N | SER | | 18 | 19.002 | 25.743 | −23.709 | 1.00 | 34.58 | N |
| ATOM | 90 | CA | SER | | 18 | 19.507 | 24.432 | −24.093 | 1.00 | 35.96 | C |
| ATOM | 91 | CB | SER | | 18 | 18.982 | 24.056 | −25.482 | 1.00 | 36.36 | C |
| ATOM | 92 | OG | SER | | 18 | 17.576 | 23.912 | −25.476 | 1.00 | 39.84 | O |
| ATOM | 93 | C | SER | | 18 | 21.027 | 24.360 | −24.104 | 1.00 | 35.79 | C |
| ATOM | 94 | O | SER | | 18 | 21.630 | 23.628 | −23.321 | 1.00 | 36.71 | O |
| ATOM | 95 | N | SER | | 19 | 21.640 | 25.131 | −24.995 | 1.00 | 34.98 | N |
| ATOM | 96 | CA | SER | | 19 | 23.089 | 25.135 | −25.149 | 1.00 | 34.34 | C |
| ATOM | 97 | CB | SER | | 19 | 23.445 | 25.408 | −26.609 | 1.00 | 35.66 | C |
| ATOM | 98 | OG | SER | | 19 | 22.974 | 26.682 | −27.008 | 1.00 | 36.30 | O |
| ATOM | 99 | C | SER | | 19 | 23.844 | 26.118 | −24.270 | 1.00 | 33.33 | C |
| ATOM | 100 | O | SER | | 19 | 25.014 | 25.901 | −23.965 | 1.00 | 33.34 | O |
| ATOM | 101 | N | GLY | | 20 | 23.187 | 27.201 | −23.869 | 1.00 | 32.22 | N |
| ATOM | 102 | CA | GLY | | 20 | 23.852 | 28.190 | −23.039 | 1.00 | 30.95 | C |
| ATOM | 103 | C | GLY | | 20 | 24.556 | 29.237 | −23.888 | 1.00 | 29.69 | C |
| ATOM | 104 | O | GLY | | 20 | 25.124 | 30.203 | −23.375 | 1.00 | 28.89 | O |
| ATOM | 105 | N | ARG | | 21 | 24.518 | 29.042 | −25.200 | 1.00 | 28.35 | N |
| ATOM | 106 | CA | ARG | | 21 | 25.151 | 29.973 | −26.117 | 1.00 | 27.16 | C |
| ATOM | 107 | CB | ARG | | 21 | 25.287 | 29.334 | −27.500 | 1.00 | 30.22 | C |
| ATOM | 108 | CG | ARG | | 21 | 25.969 | 30.212 | −28.543 | 1.00 | 32.97 | C |
| ATOM | 109 | CD | ARG | | 21 | 26.814 | 29.356 | −29.471 | 1.00 | 35.32 | C |
| ATOM | 110 | NE | ARG | | 21 | 26.099 | 28.152 | −29.877 | 1.00 | 36.40 | N |
| ATOM | 111 | CZ | ARG | | 21 | 25.252 | 28.079 | −30.898 | 1.00 | 36.53 | C |
| ATOM | 112 | NH1 | ARG | | 21 | 25.001 | 29.144 | −31.649 | 1.00 | 36.40 | N |
| ATOM | 113 | NH2 | ARG | | 21 | 24.646 | 26.931 | −31.161 | 1.00 | 38.58 | N |
| ATOM | 114 | C | ARG | | 21 | 24.332 | 31.254 | −26.208 | 1.00 | 25.64 | C |
| ATOM | 115 | O | ARG | | 21 | 23.099 | 31.222 | −26.188 | 1.00 | 23.86 | O |
| ATOM | 116 | N | VAL | | 22 | 25.022 | 32.382 | −26.305 | 1.00 | 24.47 | N |
| ATOM | 117 | CA | VAL | | 22 | 24.342 | 33.663 | −26.391 | 1.00 | 24.25 | C |
| ATOM | 118 | CB | VAL | | 22 | 25.330 | 34.837 | −26.194 | 1.00 | 26.92 | C |
| ATOM | 119 | CG1 | VAL | | 22 | 26.036 | 34.709 | −24.846 | 1.00 | 30.16 | C |
| ATOM | 120 | CG2 | VAL | | 22 | 26.347 | 34.859 | −27.320 | 1.00 | 30.14 | C |
| ATOM | 121 | C | VAL | | 22 | 23.644 | 33.833 | −27.740 | 1.00 | 21.43 | C |
| ATOM | 122 | O | VAL | | 22 | 24.064 | 33.278 | −28.758 | 1.00 | 20.14 | O |
| ATOM | 123 | N | TYR | | 23 | 22.551 | 34.581 | −27.727 | 1.00 | 20.64 | N |
| ATOM | 124 | CA | TYR | | 23 | 21.808 | 34.878 | −28.943 | 1.00 | 18.29 | C |
| ATOM | 125 | CB | TYR | | 23 | 20.758 | 33.803 | −29.247 | 1.00 | 17.05 | C |
| ATOM | 126 | CG | TYR | | 23 | 19.549 | 33.764 | −28.335 | 1.00 | 19.03 | C |
| ATOM | 127 | CD1 | TYR | | 23 | 18.353 | 34.397 | −28.694 | 1.00 | 19.87 | C |
| ATOM | 128 | CE1 | TYR | | 23 | 17.208 | 34.281 | −27.902 | 1.00 | 21.13 | C |
| ATOM | 129 | CD2 | TYR | | 23 | 19.572 | 33.025 | −27.156 | 1.00 | 22.01 | C |
| ATOM | 130 | CE2 | TYR | | 23 | 18.436 | 32.902 | −26.357 | 1.00 | 23.35 | C |
| ATOM | 131 | CZ | TYR | | 23 | 17.259 | 33.527 | −26.736 | 1.00 | 23.29 | C |
| ATOM | 132 | OH | TYR | | 23 | 16.133 | 33.361 | −25.962 | 1.00 | 23.71 | O |
| ATOM | 133 | C | TYR | | 23 | 21.155 | 36.219 | −28.686 | 1.00 | 18.43 | C |
| ATOM | 134 | O | TYR | | 23 | 21.283 | 36.771 | −27.593 | 1.00 | 18.11 | O |
| ATOM | 135 | N | TYR | | 24 | 20.472 | 36.754 | −29.685 | 1.00 | 19.30 | N |
| ATOM | 136 | CA | TYR | | 24 | 19.845 | 38.052 | −29.521 | 1.00 | 19.78 | C |
| ATOM | 137 | CB | TYR | | 24 | 20.540 | 39.077 | −30.416 | 1.00 | 23.91 | C |
| ATOM | 138 | CG | TYR | | 24 | 22.014 | 39.187 | −30.104 | 1.00 | 27.74 | C |
| ATOM | 139 | CD1 | TYR | | 24 | 22.921 | 38.242 | −30.590 | 1.00 | 29.88 | C |
| ATOM | 140 | CE1 | TYR | | 24 | 24.267 | 38.284 | −30.233 | 1.00 | 32.47 | C |
| ATOM | 141 | CD2 | TYR | | 24 | 22.494 | 40.182 | −29.253 | 1.00 | 29.73 | C |
| ATOM | 142 | CE2 | TYR | | 24 | 23.840 | 40.233 | −28.888 | 1.00 | 33.45 | C |
| ATOM | 143 | CZ | TYR | | 24 | 24.717 | 39.279 | −29.382 | 1.00 | 34.29 | C |
| ATOM | 144 | OH | TYR | | 24 | 26.044 | 39.314 | −29.017 | 1.00 | 38.08 | O |
| ATOM | 145 | C | TYR | | 24 | 18.364 | 37.983 | −29.815 | 1.00 | 18.94 | C |
| ATOM | 146 | O | TYR | | 24 | 17.929 | 37.263 | −30.709 | 1.00 | 16.86 | O |
| ATOM | 147 | N | PHE | | 25 | 17.601 | 38.734 | −29.033 | 1.00 | 18.38 | N |
| ATOM | 148 | CA | PHE | | 25 | 16.153 | 38.771 | −29.145 | 1.00 | 19.81 | C |
| ATOM | 149 | CB | PHE | | 25 | 15.542 | 38.147 | −27.885 | 1.00 | 20.22 | C |
| ATOM | 150 | CG | PHE | | 25 | 14.066 | 38.350 | −27.758 | 1.00 | 20.32 | C |
| ATOM | 151 | CD1 | PHE | | 25 | 13.182 | 37.717 | −28.631 | 1.00 | 22.39 | C |
| ATOM | 152 | CD2 | PHE | | 25 | 13.555 | 39.189 | −26.771 | 1.00 | 20.95 | C |
| ATOM | 153 | CE1 | PHE | | 25 | 11.802 | 37.920 | −28.522 | 1.00 | 23.14 | C |
| ATOM | 154 | CE2 | PHE | | 25 | 12.179 | 39.399 | −26.653 | 1.00 | 22.17 | C |
| ATOM | 155 | CZ | PHE | | 25 | 11.303 | 38.763 | −27.532 | 1.00 | 22.96 | C |
| ATOM | 156 | C | PHE | | 25 | 15.704 | 40.219 | −29.279 | 1.00 | 19.13 | C |
| ATOM | 157 | O | PHE | | 25 | 16.261 | 41.108 | −28.632 | 1.00 | 20.48 | O |
| ATOM | 158 | N | ASN | | 26 | 14.703 | 40.448 | −30.124 | 1.00 | 17.74 | N |
| ATOM | 159 | CA | ASN | | 26 | 14.155 | 41.790 | −30.338 | 1.00 | 17.69 | C |
| ATOM | 160 | CB | ASN | | 26 | 14.120 | 42.119 | −31.834 | 1.00 | 16.86 | C |
| ATOM | 161 | CG | ASN | | 26 | 13.696 | 43.552 | −32.105 | 1.00 | 17.44 | C |
| ATOM | 162 | OD1 | ASN | | 26 | 12.601 | 43.971 | −31.729 | 1.00 | 17.21 | O |
| ATOM | 163 | ND2 | ASN | | 26 | 14.565 | 44.310 | −32.760 | 1.00 | 19.43 | N |
| ATOM | 164 | C | ASN | | 26 | 12.742 | 41.780 | −29.773 | 1.00 | 15.00 | C |
| ATOM | 165 | O | ASN | | 26 | 11.885 | 41.044 | −30.263 | 1.00 | 16.36 | O |
| ATOM | 166 | N | HIS | | 27 | 12.496 | 42.594 | −28.747 | 1.00 | 14.90 | N |
| ATOM | 167 | CA | HIS | | 27 | 11.186 | 42.625 | −28.099 | 1.00 | 15.79 | C |
| ATOM | 168 | CB | HIS | | 27 | 11.317 | 43.144 | −26.658 | 1.00 | 16.75 | C |
| ATOM | 169 | CG | HIS | | 27 | 11.870 | 44.533 | −26.558 | 1.00 | 20.66 | C |
| ATOM | 170 | CD2 | HIS | | 27 | 13.122 | 44.976 | −26.297 | 1.00 | 20.21 | C |
| ATOM | 171 | ND1 | HIS | | 27 | 11.095 | 45.659 | −26.741 | 1.00 | 24.85 | N |
| ATOM | 172 | CE1 | HIS | | 27 | 11.847 | 46.736 | −26.596 | 1.00 | 23.42 | C |
| ATOM | 173 | NE2 | HIS | | 27 | 13.081 | 46.349 | −26.328 | 1.00 | 24.68 | N |
| ATOM | 174 | C | HIS | | 27 | 10.106 | 43.408 | −28.837 | 1.00 | 16.49 | C |
| ATOM | 175 | O | HIS | | 27 | 8.977 | 43.505 | −28.361 | 1.00 | 17.21 | O |
| ATOM | 176 | N | ILE | | 28 | 10.452 | 43.965 | −29.992 | 1.00 | 16.77 | N |
| ATOM | 177 | CA | ILE | | 28 | 9.480 | 44.701 | −30.792 | 1.00 | 16.22 | C |
| ATOM | 178 | CB | ILE | | 28 | 10.096 | 45.980 | −31.395 | 1.00 | 17.94 | C |
| ATOM | 179 | CG2 | ILE | | 28 | 9.044 | 46.714 | −32.239 | 1.00 | 16.31 | C |
| ATOM | 180 | CG1 | ILE | | 28 | 10.580 | 46.907 | −30.276 | 1.00 | 17.14 | C |
| ATOM | 181 | CD1 | ILE | | 28 | 11.360 | 48.124 | −30.781 | 1.00 | 18.58 | C |
| ATOM | 182 | C | ILE | | 28 | 8.968 | 43.809 | −31.926 | 1.00 | 17.18 | C |
| ATOM | 183 | O | ILE | | 28 | 7.771 | 43.798 | −32.226 | 1.00 | 17.90 | O |
| ATOM | 184 | N | THR | | 29 | 9.871 | 43.043 | −32.534 | 1.00 | 16.23 | N |
| ATOM | 185 | CA | THR | | 29 | 9.516 | 42.147 | −33.645 | 1.00 | 16.60 | C |
| ATOM | 186 | CB | THR | | 29 | 10.587 | 42.162 | −34.740 | 1.00 | 16.74 | C |
| ATOM | 187 | OG1 | THR | | 29 | 11.793 | 41.602 | −34.203 | 1.00 | 16.47 | O |
| ATOM | 188 | CG2 | THR | | 29 | 10.865 | 43.580 | −35.225 | 1.00 | 16.57 | C |
| ATOM | 189 | C | THR | | 29 | 9.394 | 40.689 | −33.212 | 1.00 | 16.39 | C |
| ATOM | 190 | O | THR | | 29 | 8.880 | 39.852 | −33.962 | 1.00 | 15.21 | O |
| ATOM | 191 | N | ASN | | 30 | 9.883 | 40.391 | −32.010 | 1.00 | 16.48 | N |
| ATOM | 192 | CA | ASN | | 30 | 9.880 | 39.033 | −31.474 | 1.00 | 15.89 | C |
| ATOM | 193 | CB | ASN | | 30 | 8.449 | 38.478 | −31.424 | 1.00 | 17.49 | C |
| ATOM | 194 | CG | ASN | | 30 | 7.663 | 39.011 | −30.230 | 1.00 | 19.11 | C |
| ATOM | 195 | OD1 | ASN | | 30 | 6.426 | 39.053 | −30.241 | 1.00 | 19.17 | O |
| ATOM | 196 | ND2 | ASN | | 30 | 8.383 | 39.410 | −29.186 | 1.00 | 15.19 | N |
| ATOM | 197 | C | ASN | | 30 | 10.810 | 38.109 | −32.264 | 1.00 | 15.49 | C |
| ATOM | 198 | O | ASN | | 30 | 10.717 | 36.881 | −32.175 | 1.00 | 14.57 | O |
| ATOM | 199 | N | ALA | | 31 | 11.720 | 38.696 | −33.035 | 1.00 | 14.85 | N |
| ATOM | 200 | CA | ALA | | 31 | 12.679 | 37.886 | −33.786 | 1.00 | 15.31 | C |
| ATOM | 201 | CB | ALA | | 31 | 13.242 | 38.671 | −34.964 | 1.00 | 14.57 | C |
| ATOM | 202 | C | ALA | | 31 | 13.821 | 37.467 | −32.863 | 1.00 | 16.03 | C |
| ATOM | 203 | O | ALA | | 31 | 14.117 | 38.139 | −31.876 | 1.00 | 15.69 | O |
| ATOM | 204 | N | SER | | 32 | 14.453 | 36.343 | −33.181 | 1.00 | 15.20 | N |
| ATOM | 205 | CA | SER | | 32 | 15.589 | 35.868 | −32.402 | 1.00 | 17.81 | C |
| ATOM | 206 | CB | SER | | 32 | 15.152 | 34.816 | −31.374 | 1.00 | 16.12 | C |
| ATOM | 207 | OG | SER | | 32 | 14.361 | 33.801 | −31.965 | 1.00 | 22.34 | O |
| ATOM | 208 | C | SER | | 32 | 16.613 | 35.298 | −33.378 | 1.00 | 18.35 | C |
| ATOM | 209 | O | SER | | 32 | 16.249 | 34.626 | −34.343 | 1.00 | 18.48 | O |
| ATOM | 210 | N | GLN | | 33 | 17.887 | 35.590 | −33.132 | 1.00 | 19.94 | N |
| ATOM | 211 | CA | GLN | | 33 | 18.975 | 35.134 | −33.999 | 1.00 | 21.00 | C |
| ATOM | 212 | CB | GLN | | 33 | 19.227 | 36.179 | −35.093 | 1.00 | 21.27 | C |
| ATOM | 213 | CG | GLN | | 33 | 19.507 | 37.579 | −34.535 | 1.00 | 26.19 | C |
| ATOM | 214 | CD | GLN | | 33 | 19.799 | 38.613 | −35.614 | 1.00 | 30.29 | C |
| ATOM | 215 | OE1 | GLN | | 33 | 18.997 | 38.825 | −36.522 | 1.00 | 32.87 | O |
| ATOM | 216 | NE2 | GLN | | 33 | 20.953 | 39.265 | −35.512 | 1.00 | 30.34 | N |
| ATOM | 217 | C | GLN | | 33 | 20.265 | 34.930 | −33.197 | 1.00 | 21.95 | C |
| ATOM | 218 | O | GLN | | 33 | 20.433 | 35.512 | −32.126 | 1.00 | 19.90 | O |
| ATOM | 219 | N | TRP | | 34 | 21.183 | 34.124 | −33.732 | 1.00 | 23.88 | N |
| ATOM | 220 | CA | TRP | | 34 | 22.455 | 33.860 | −33.058 | 1.00 | 22.59 | C |
| ATOM | 221 | CB | TRP | | 34 | 23.123 | 32.600 | −33.633 | 1.00 | 20.70 | C |
| ATOM | 222 | CG | TRP | | 34 | 22.401 | 31.321 | −33.325 | 1.00 | 19.59 | C |
| ATOM | 223 | CD2 | TRP | | 34 | 22.196 | 30.738 | −32.031 | 1.00 | 20.78 | C |
| ATOM | 224 | CE2 | TRP | | 34 | 21.455 | 29.550 | −32.221 | 1.00 | 21.49 | C |
| ATOM | 225 | CE3 | TRP | | 34 | 22.566 | 31.105 | −30.729 | 1.00 | 21.77 | C |
| ATOM | 226 | CD1 | TRP | | 34 | 21.795 | 30.484 | −34.221 | 1.00 | 21.09 | C |
| ATOM | 227 | NE1 | TRP | | 34 | 21.225 | 29.421 | −33.565 | 1.00 | 20.42 | N |
| ATOM | 228 | CZ2 | TRP | | 34 | 21.073 | 28.725 | −31.156 | 1.00 | 22.28 | C |
| ATOM | 229 | CZ3 | TRP | | 34 | 22.187 | 30.283 | −29.668 | 1.00 | 23.47 | C |
| ATOM | 230 | CH2 | TRP | | 34 | 21.446 | 29.107 | −29.891 | 1.00 | 24.65 | C |
| ATOM | 231 | C | TRP | | 34 | 23.439 | 35.028 | −33.143 | 1.00 | 24.82 | C |
| ATOM | 232 | O | TRP | | 34 | 24.083 | 35.370 | −32.152 | 1.00 | 23.87 | O |
| ATOM | 233 | N | GLU | | 35 | 23.556 | 35.645 | −34.317 | 1.00 | 28.44 | N |
| ATOM | 234 | CA | GLU | | 35 | 24.488 | 36.756 | −34.485 | 1.00 | 32.89 | C |
| ATOM | 235 | CB | GLU | | 35 | 24.767 | 37.020 | −35.962 | 1.00 | 34.03 | C |
| ATOM | 236 | CG | GLU | | 35 | 25.382 | 35.862 | −36.713 | 1.00 | 35.42 | C |
| ATOM | 237 | CD | GLU | | 35 | 26.020 | 36.305 | −38.014 | 1.00 | 35.75 | C |
| ATOM | 238 | OE1 | GLU | | 35 | 26.122 | 35.471 | −38.935 | 1.00 | 35.53 | O |
| ATOM | 239 | OE2 | GLU | | 35 | 26.432 | 37.486 | −38.106 | 1.00 | 35.56 | O |
| ATOM | 240 | C | GLU | | 35 | 24.039 | 38.062 | −33.857 | 1.00 | 36.99 | C |
| ATOM | 241 | O | GLU | | 35 | 22.844 | 38.323 | −33.713 | 1.00 | 35.51 | O |
| ATOM | 242 | N | ARG | | 36 | 25.020 | 38.882 | −33.490 | 1.00 | 40.29 | N |
| ATOM | 243 | CA | ARG | | 36 | 24.751 | 40.182 | −32.902 | 1.00 | 44.94 | C |
| ATOM | 244 | CB | ARG | | 36 | 26.060 | 40.821 | −32.430 | 1.00 | 45.80 | C |
| ATOM | 245 | CG | ARG | | 36 | 25.897 | 41.845 | −31.320 | 1.00 | 47.56 | C |
| ATOM | 246 | CD | ARG | | 36 | 27.212 | 42.053 | −30.584 | 1.00 | 49.01 | C |
| ATOM | 247 | NE | ARG | | 36 | 27.039 | 42.819 | −29.353 | 1.00 | 49.91 | N |
| ATOM | 248 | CZ | ARG | | 36 | 26.760 | 44.117 | −29.310 | 1.00 | 51.00 | C |
| ATOM | 249 | NH1 | ARG | | 36 | 26.625 | 44.809 | −30.435 | 1.00 | 50.81 | N |
| ATOM | 250 | NH2 | ARG | | 36 | 26.611 | 44.723 | −28.140 | 1.00 | 51.83 | N |
| ATOM | 251 | C | ARG | | 36 | 24.124 | 40.987 | −34.037 | 1.00 | 47.51 | C |
| ATOM | 252 | O | ARG | | 36 | 24.741 | 41.183 | −35.084 | 1.00 | 48.09 | O |
| ATOM | 253 | N | PRO | | 37 | 22.882 | 41.456 | −33.844 | 1.00 | 49.48 | N |
| ATOM | 254 | CD | PRO | | 37 | 22.216 | 41.570 | −32.536 | 1.00 | 49.60 | C |
| ATOM | 255 | CA | PRO | | 37 | 22.160 | 42.233 | −34.855 | 1.00 | 51.54 | C |
| ATOM | 256 | CB | PRO | | 37 | 20.945 | 42.750 | −34.087 | 1.00 | 50.70 | C |
| ATOM | 257 | CG | PRO | | 37 | 21.456 | 42.861 | −32.688 | 1.00 | 50.69 | C |
| ATOM | 258 | C | PRO | | 37 | 22.961 | 43.354 | −35.509 | 1.00 | 53.52 | C |
| ATOM | 259 | O | PRO | | 37 | 23.708 | 44.075 | −34.842 | 1.00 | 54.16 | O |
| ATOM | 260 | N | SER | | 38 | 22.793 | 43.485 | −36.822 | 1.00 | 55.71 | N |
| ATOM | 261 | CA | SER | | 38 | 23.475 | 44.511 | −37.604 | 1.00 | 57.41 | C |
| ATOM | 262 | CB | SER | | 38 | 22.844 | 45.880 | −37.333 | 1.00 | 57.75 | C |
| ATOM | 263 | OG | SER | | 38 | 21.460 | 45.867 | −37.637 | 1.00 | 58.08 | O |
| ATOM | 264 | C | SER | | 38 | 24.969 | 44.565 | −37.301 | 1.00 | 58.14 | C |
| ATOM | 265 | O | SER | | 38 | 25.779 | 44.861 | −38.181 | 1.00 | 59.14 | O |
| ATOM | 266 | N | GLU | | 51 | 14.614 | 56.346 | −39.127 | 1.00 | 53.04 | N |
| ATOM | 267 | CA | GLU | | 51 | 13.987 | 55.185 | −39.745 | 1.00 | 52.49 | C |
| ATOM | 268 | CB | GLU | | 51 | 12.875 | 54.642 | −38.844 | 1.00 | 52.96 | C |
| ATOM | 269 | CG | GLU | | 51 | 11.632 | 55.517 | −38.809 | 1.00 | 52.89 | C |
| ATOM | 270 | CD | GLU | | 51 | 10.524 | 54.925 | −37.962 | 1.00 | 52.81 | C |
| ATOM | 271 | OE1 | GLU | | 51 | 9.410 | 55.487 | −37.969 | 1.00 | 51.84 | O |
| ATOM | 272 | OE2 | GLU | | 51 | 10.765 | 53.901 | −37.287 | 1.00 | 53.59 | O |
| ATOM | 273 | C | GLU | | 51 | 13.395 | 55.574 | −41.098 | 1.00 | 51.96 | C |
| ATOM | 274 | O | GLU | | 51 | 13.185 | 56.757 | −41.373 | 1.00 | 52.52 | O |
| ATOM | 275 | N | PRO | | 52 | 13.108 | 54.581 | −41.958 | 1.00 | 50.63 | N |
| ATOM | 276 | CD | PRO | | 52 | 13.211 | 53.126 | −41.746 | 1.00 | 50.48 | C |
| ATOM | 277 | CA | PRO | | 52 | 12.537 | 54.873 | −43.275 | 1.00 | 48.30 | C |
| ATOM | 278 | CB | PRO | | 52 | 12.508 | 53.503 | −43.943 | 1.00 | 48.86 | C |
| ATOM | 279 | CG | PRO | | 52 | 12.256 | 52.585 | −42.787 | 1.00 | 48.97 | C |
| ATOM | 280 | C | PRO | | 52 | 11.148 | 55.489 | −43.157 | 1.00 | 46.35 | C |
| ATOM | 281 | O | PRO | | 52 | 10.421 | 55.221 | −42.201 | 1.00 | 46.27 | O |
| ATOM | 282 | N | ALA | | 53 | 10.786 | 56.317 | −44.129 | 1.00 | 43.75 | N |
| ATOM | 283 | CA | ALA | | 53 | 9.482 | 56.960 | −44.127 | 1.00 | 40.35 | C |
| ATOM | 284 | CB | ALA | | 53 | 9.435 | 58.043 | −45.192 | 1.00 | 42.00 | C |
| ATOM | 285 | C | ALA | | 53 | 8.405 | 55.921 | −44.391 | 1.00 | 37.97 | C |
| ATOM | 286 | O | ALA | | 53 | 7.299 | 56.001 | −43.856 | 1.00 | 36.28 | O |
| ATOM | 287 | N | ARG | | 54 | 8.738 | 54.941 | −45.224 | 1.00 | 35.90 | N |
| ATOM | 288 | CA | ARG | | 54 | 7.802 | 53.881 | −45.572 | 1.00 | 32.70 | C |
| ATOM | 289 | CB | ARG | | 54 | 7.123 | 54.200 | −46.906 | 1.00 | 35.72 | C |
| ATOM | 290 | CG | ARG | | 54 | 6.361 | 55.516 | −46.929 | 1.00 | 39.13 | C |
| ATOM | 291 | CD | ARG | | 54 | 6.203 | 56.026 | −48.354 | 1.00 | 42.35 | C |
| ATOM | 292 | NE | ARG | | 54 | 5.461 | 55.100 | −49.205 | 1.00 | 43.72 | N |
| ATOM | 293 | CZ | ARG | | 54 | 4.174 | 54.810 | −49.048 | 1.00 | 44.96 | C |
| ATOM | 294 | NH1 | ARG | | 54 | 3.479 | 55.374 | −48.069 | 1.00 | 46.53 | N |
| ATOM | 295 | NH2 | ARG | | 54 | 3.581 | 53.962 | −49.876 | 1.00 | 46.08 | N |
| ATOM | 296 | C | ARG | | 54 | 8.514 | 52.540 | −45.690 | 1.00 | 29.25 | C |
| ATOM | 297 | O | ARG | | 54 | 9.726 | 52.477 | −45.898 | 1.00 | 28.19 | O |
| ATOM | 298 | N | VAL | | 55 | 7.748 | 51.468 | −45.546 | 1.00 | 26.67 | N |
| ATOM | 299 | CA | VAL | | 55 | 8.288 | 50.124 | −45.675 | 1.00 | 24.08 | C |
| ATOM | 300 | CB | VAL | | 55 | 8.558 | 49.455 | −44.299 | 1.00 | 22.45 | C |
| ATOM | 301 | CG1 | VAL | | 55 | 9.473 | 50.325 | −43.462 | 1.00 | 22.61 | C |
| ATOM | 302 | CG2 | VAL | | 55 | 7.245 | 49.189 | −43.573 | 1.00 | 21.69 | C |
| ATOM | 303 | C | VAL | | 55 | 7.260 | 49.282 | −46.408 | 1.00 | 23.28 | C |
| ATOM | 304 | O | VAL | | 55 | 6.081 | 49.634 | −46.472 | 1.00 | 23.48 | O |
| ATOM | 305 | N | ARG | | 56 | 7.716 | 48.176 | −46.979 | 1.00 | 20.35 | N |
| ATOM | 306 | CA | ARG | | 56 | 6.826 | 47.269 | −47.673 | 1.00 | 18.76 | C |
| ATOM | 307 | CB | ARG | | 56 | 7.222 | 47.125 | −49.144 | 1.00 | 19.12 | C |
| ATOM | 308 | CG | ARG | | 56 | 6.277 | 46.227 | −49.917 | 1.00 | 21.28 | C |
| ATOM | 309 | CD | ARG | | 56 | 6.693 | 46.081 | −51.373 | 1.00 | 23.24 | C |
| ATOM | 310 | NE | ARG | | 56 | 5.724 | 45.290 | −52.130 | 1.00 | 24.12 | N |
| ATOM | 311 | CZ | ARG | | 56 | 5.795 | 45.082 | −53.442 | 1.00 | 25.74 | C |
| ATOM | 312 | NH1 | ARG | | 56 | 6.791 | 45.605 | −54.145 | 1.00 | 25.07 | N |
| ATOM | 313 | NH2 | ARG | | 56 | 4.871 | 44.353 | −54.052 | 1.00 | 22.75 | N |
| ATOM | 314 | C | ARG | | 56 | 6.975 | 45.934 | −46.963 | 1.00 | 18.38 | C |
| ATOM | 315 | O | ARG | | 56 | 8.092 | 45.483 | −46.708 | 1.00 | 17.93 | O |
| ATOM | 316 | N | CYS | | 57 | 5.851 | 45.313 | −46.630 | 1.00 | 17.73 | N |
| ATOM | 317 | CA | CYS | | 57 | 5.872 | 44.033 | −45.941 | 1.00 | 17.76 | C |
| ATOM | 318 | CB | CYS | | 57 | 5.571 | 44.217 | −44.448 | 1.00 | 17.32 | C |
| ATOM | 319 | SG | CYS | | 57 | 6.751 | 45.199 | −43.534 | 1.00 | 18.68 | S |
| ATOM | 320 | C | CYS | | 57 | 4.849 | 43.063 | −46.479 | 1.00 | 17.75 | C |
| ATOM | 321 | O | CYS | | 57 | 3.862 | 43.450 | −47.105 | 1.00 | 19.12 | O |
| ATOM | 322 | N | SER | | 58 | 5.102 | 41.789 | −46.215 | 1.00 | 16.21 | N |
| ATOM | 323 | CA | SER | | 58 | 4.187 | 40.723 | −46.562 | 1.00 | 15.73 | C |
| ATOM | 324 | CB | SER | | 58 | 4.840 | 39.694 | −47.489 | 1.00 | 15.00 | C |
| ATOM | 325 | OG | SER | | 58 | 5.017 | 40.224 | −48.787 | 1.00 | 16.34 | O |
| ATOM | 326 | C | SER | | 58 | 3.954 | 40.103 | −45.197 | 1.00 | 16.55 | C |
| ATOM | 327 | O | SER | | 58 | 4.755 | 40.314 | −44.276 | 1.00 | 15.97 | O |
| ATOM | 328 | N | HIS | | 59 | 2.865 | 39.364 | −45.042 | 1.00 | 15.13 | N |
| ATOM | 329 | CA | HIS | | 59 | 2.615 | 38.725 | −43.766 | 1.00 | 15.74 | C |
| ATOM | 330 | CB | HIS | | 59 | 1.967 | 39.713 | −42.776 | 1.00 | 17.79 | C |
| ATOM | 331 | CG | HIS | | 59 | 0.485 | 39.873 | −42.942 | 1.00 | 20.01 | C |
| ATOM | 332 | CD2 | HIS | | 59 | −0.524 | 39.776 | −42.043 | 1.00 | 20.23 | C |
| ATOM | 333 | ND1 | HIS | | 59 | −0.106 | 40.191 | −44.146 | 1.00 | 23.53 | N |
| ATOM | 334 | CE1 | HIS | | 59 | −1.415 | 40.282 | −43.983 | 1.00 | 20.29 | C |
| ATOM | 335 | NE2 | HIS | | 59 | −1.694 | 40.036 | −42.716 | 1.00 | 24.94 | N |
| ATOM | 336 | C | HIS | | 59 | 1.758 | 37.483 | −43.916 | 1.00 | 15.38 | C |
| ATOM | 337 | O | HIS | | 59 | 1.115 | 37.254 | −44.946 | 1.00 | 15.49 | O |
| ATOM | 338 | N | LEU | | 60 | 1.790 | 36.660 | −42.883 | 1.00 | 13.98 | N |
| ATOM | 339 | CA | LEU | | 60 | 1.005 | 35.448 | −42.844 | 1.00 | 14.87 | C |
| ATOM | 340 | CB | LEU | | 60 | 1.916 | 34.224 | −42.809 | 1.00 | 14.46 | C |
| ATOM | 341 | CG | LEU | | 60 | 1.245 | 32.855 | −42.956 | 1.00 | 15.20 | C |
| ATOM | 342 | CD1 | LEU | | 60 | 2.300 | 31.812 | −43.313 | 1.00 | 16.63 | C |
| ATOM | 343 | CD2 | LEU | | 60 | 0.522 | 32.480 | −41.668 | 1.00 | 16.52 | C |
| ATOM | 344 | C | LEU | | 60 | 0.236 | 35.591 | −41.545 | 1.00 | 16.69 | C |
| ATOM | 345 | O | LEU | | 60 | 0.829 | 35.697 | −40.469 | 1.00 | 14.62 | O |
| ATOM | 346 | N | LEU | | 61 | −1.087 | 35.620 | −41.656 | 1.00 | 16.04 | N |
| ATOM | 347 | CA | LEU | | 61 | −1.949 | 35.776 | −40.493 | 1.00 | 17.56 | C |
| ATOM | 348 | CB | LEU | | 61 | −3.006 | 36.854 | −40.772 | 1.00 | 17.54 | C |
| ATOM | 349 | CG | LEU | | 61 | −4.111 | 37.017 | −39.717 | 1.00 | 19.27 | C |
| ATOM | 350 | CD1 | LEU | | 61 | −3.504 | 37.490 | −38.397 | 1.00 | 20.49 | C |
| ATOM | 351 | CD2 | LEU | | 61 | −5.145 | 38.019 | −40.207 | 1.00 | 20.78 | C |
| ATOM | 352 | C | LEU | | 61 | −2.660 | 34.482 | −40.127 | 1.00 | 16.44 | C |
| ATOM | 353 | O | LEU | | 61 | −3.180 | 33.782 | −40.993 | 1.00 | 16.25 | O |
| ATOM | 354 | N | VAL | | 62 | −2.658 | 34.154 | −38.838 | 1.00 | 16.65 | N |
| ATOM | 355 | CA | VAL | | 62 | −3.376 | 32.985 | −38.365 | 1.00 | 17.17 | C |
| ATOM | 356 | CB | VAL | | 62 | −2.461 | 31.943 | −37.700 | 1.00 | 18.11 | C |
| ATOM | 357 | CG1 | VAL | | 62 | −3.313 | 30.817 | −37.129 | 1.00 | 18.51 | C |
| ATOM | 358 | CG2 | VAL | | 62 | −1.484 | 31.380 | −38.717 | 1.00 | 18.42 | C |
| ATOM | 359 | C | VAL | | 62 | −4.341 | 33.545 | −37.326 | 1.00 | 19.26 | C |
| ATOM | 360 | O | VAL | | 62 | −3.924 | 34.103 | −36.307 | 1.00 | 16.64 | O |
| ATOM | 361 | N | LYS | | 63 | −5.633 | 33.434 | −37.607 | 1.00 | 19.67 | N |
| ATOM | 362 | CA | LYS | | 63 | −6.637 | 33.950 | −36.689 | 1.00 | 22.74 | C |
| ATOM | 363 | CB | LYS | | 63 | −7.897 | 34.372 | −37.459 | 1.00 | 22.84 | C |
| ATOM | 364 | CG | LYS | | 63 | −7.720 | 35.615 | −38.333 | 1.00 | 25.21 | C |
| ATOM | 365 | CD | LYS | | 63 | −9.057 | 36.064 | −38.930 | 1.00 | 26.01 | C |
| ATOM | 366 | CE | LYS | | 63 | −8.921 | 37.343 | −39.734 | 1.00 | 26.44 | C |
| ATOM | 367 | NZ | LYS | | 63 | −10.230 | 37.778 | −40.315 | 1.00 | 28.10 | N |
| ATOM | 368 | C | LYS | | 63 | −6.997 | 32.903 | −35.643 | 1.00 | 22.30 | C |
| ATOM | 369 | O | LYS | | 63 | −6.686 | 31.721 | −35.796 | 1.00 | 21.61 | O |
| ATOM | 370 | N | HIS | | 64 | −7.639 | 33.351 | −34.571 | 1.00 | 23.65 | N |
| ATOM | 371 | CA | HIS | | 64 | −8.071 | 32.454 | −33.507 | 1.00 | 25.29 | C |
| ATOM | 372 | CB | HIS | | 64 | −7.049 | 32.439 | −32.358 | 1.00 | 22.87 | C |
| ATOM | 373 | CG | HIS | | 64 | −6.606 | 33.798 | −31.910 | 1.00 | 23.56 | C |
| ATOM | 374 | CD2 | HIS | | 64 | −5.466 | 34.487 | −32.154 | 1.00 | 22.70 | C |
| ATOM | 375 | ND1 | HIS | | 64 | −7.373 | 34.604 | −31.098 | 1.00 | 23.03 | N |
| ATOM | 376 | CE1 | HIS | | 64 | −6.724 | 35.730 | −30.859 | 1.00 | 25.33 | C |
| ATOM | 377 | NE2 | HIS | | 64 | −5.564 | 35.684 | −31.489 | 1.00 | 23.80 | N |
| ATOM | 378 | C | HIS | | 64 | −9.448 | 32.894 | −33.021 | 1.00 | 26.27 | C |
| ATOM | 379 | O | HIS | | 64 | −9.968 | 33.916 | −33.472 | 1.00 | 25.01 | O |
| ATOM | 380 | N | SER | | 65 | −10.035 | 32.122 | −32.109 | 1.00 | 27.94 | N |
| ATOM | 381 | CA | SER | | 65 | −11.371 | 32.420 | −31.593 | 1.00 | 29.00 | C |
| ATOM | 382 | CB | SER | | 65 | −11.789 | 31.355 | −30.575 | 1.00 | 29.57 | C |
| ATOM | 383 | CG | SER | | 65 | −10.942 | 31.369 | −29.440 | 1.00 | 32.41 | O |
| ATOM | 384 | C | SER | | 65 | −11.557 | 33.811 | −30.984 | 1.00 | 30.15 | C |
| ATOM | 385 | O | SER | | 65 | −12.681 | 34.313 | −30.926 | 1.00 | 30.15 | O |
| ATOM | 386 | N | GLN | | 66 | −10.474 | 34.438 | −30.527 | 1.00 | 29.29 | N |
| ATOM | 387 | CA | GLN | | 66 | −10.585 | 35.772 | −29.943 | 1.00 | 30.11 | C |
| ATOM | 388 | CB | GLN | | 66 | −9.696 | 35.904 | −28.698 | 1.00 | 31.22 | C |
| ATOM | 389 | CG | GLN | | 66 | −10.186 | 35.118 | −27.487 | 1.00 | 34.12 | C |
| ATOM | 390 | CD | GLN | | 66 | −9.496 | 35.542 | −26.196 | 1.00 | 36.25 | C |
| ATOM | 391 | OE1 | GLN | | 66 | −9.646 | 36.681 | −25.738 | 1.00 | 34.02 | O |
| ATOM | 392 | NE2 | GLN | | 66 | −8.731 | 34.627 | −25.605 | 1.00 | 36.53 | N |
| ATOM | 393 | C | GLN | | 66 | −10.247 | 36.890 | −30.925 | 1.00 | 29.93 | C |
| ATOM | 394 | O | GLN | | 66 | −10.202 | 38.061 | −30.546 | 1.00 | 31.18 | O |
| ATOM | 395 | N | SER | | 67 | −10.005 | 36.538 | −32.185 | 1.00 | 30.12 | N |
| ATOM | 396 | CA | SER | | 67 | −9.691 | 37.547 | −33.191 | 1.00 | 29.93 | C |
| ATOM | 397 | CB | SER | | 67 | −9.318 | 36.886 | −34.521 | 1.00 | 29.06 | C |
| ATOM | 398 | OG | SER | | 67 | −8.066 | 36.231 | −34.434 | 1.00 | 26.26 | O |
| ATOM | 399 | C | SER | | 67 | −10.900 | 38.457 | −33.392 | 1.00 | 31.50 | C |
| ATOM | 400 | O | SER | | 67 | −12.043 | 38.015 | −33.266 | 1.00 | 31.79 | O |
| ATOM | 401 | N | ARG | | 68 | −10.639 | 39.724 | −33.701 | 1.00 | 33.47 | N |
| ATOM | 402 | CA | ARG | | 68 | −11.701 | 40.703 | −33.919 | 1.00 | 36.42 | C |
| ATOM | 403 | CB | ARG | | 68 | −11.123 | 41.964 | −34.562 | 1.00 | 38.86 | C |
| ATOM | 404 | CG | ARG | | 68 | −11.507 | 43.258 | −33.853 | 1.00 | 44.11 | C |
| ATOM | 405 | CD | ARG | | 68 | −11.086 | 43.235 | −32.385 | 1.00 | 47.39 | C |
| ATOM | 406 | NE | ARG | | 68 | −11.236 | 44.538 | −31.739 | 1.00 | 49.33 | N |
| ATOM | 407 | CZ | ARG | | 68 | −10.983 | 44.769 | −30.454 | 1.00 | 50.65 | C |
| ATOM | 408 | NH1 | ARG | | 68 | −10.568 | 43.782 | −29.670 | 1.00 | 50.98 | N |
| ATOM | 409 | NH2 | ARG | | 68 | −11.138 | 45.987 | −29.952 | 1.00 | 51.60 | N |
| ATOM | 410 | C | ARG | | 68 | −12.800 | 40.125 | −34.804 | 1.00 | 36.99 | C |
| ATOM | 411 | O | ARG | | 68 | −13.988 | 40.288 | −34.522 | 1.00 | 38.05 | O |
| ATOM | 412 | N | ARG | | 69 | −12.398 | 39.447 | −35.875 | 1.00 | 36.39 | N |
| ATOM | 413 | CA | ARG | | 69 | −13.343 | 38.828 | −36.798 | 1.00 | 35.06 | C |
| ATOM | 414 | CB | ARG | | 69 | −13.391 | 39.617 | −38.109 | 1.00 | 37.95 | C |
| ATOM | 415 | CG | ARG | | 69 | −14.489 | 39.177 | −39.070 | 1.00 | 40.90 | C |
| ATOM | 416 | CD | ARG | | 69 | −14.596 | 40.125 | −40.261 | 1.00 | 44.53 | C |
| ATOM | 417 | NE | ARG | | 69 | −13.477 | 40.000 | −41.194 | 1.00 | 46.91 | N |
| ATOM | 418 | CZ | ARG | | 69 | −13.299 | 38.967 | −42.013 | 1.00 | 47.74 | C |
| ATOM | 419 | NH1 | ARG | | 69 | −14.168 | 37.963 | −42.017 | 1.00 | 48.01 | N |
| ATOM | 420 | NH2 | ARG | | 69 | −12.255 | 38.938 | −42.831 | 1.00 | 47.70 | N |
| ATOM | 421 | C | ARG | | 69 | −12.878 | 37.397 | −37.058 | 1.00 | 33.95 | C |
| ATOM | 422 | O | ARG | | 69 | −12.099 | 37.146 | −37.980 | 1.00 | 33.71 | O |
| ATOM | 423 | N | PRO | | 70 | −13.348 | 36.440 | −36.239 | 1.00 | 32.21 | N |
| ATOM | 424 | CD | PRO | | 70 | −14.265 | 36.640 | −35.103 | 1.00 | 32.12 | C |
| ATOM | 425 | CA | PRO | | 70 | −12.982 | 35.024 | −36.366 | 1.00 | 30.96 | C |
| ATOM | 426 | CB | PRO | | 70 | −13.549 | 34.410 | −35.088 | 1.00 | 32.72 | C |
| ATOM | 427 | CG | PRO | | 70 | −14.768 | 35.240 | −34.843 | 1.00 | 31.84 | C |
| ATOM | 428 | C | PRO | | 70 | −13.491 | 34.332 | −37.626 | 1.00 | 29.75 | C |
| ATOM | 429 | O | PRO | | 70 | −14.186 | 33.314 | −37.555 | 1.00 | 28.97 | O |
| ATOM | 430 | N | SER | | 71 | −13.122 | 34.880 | −38.779 | 1.00 | 27.85 | N |
| ATOM | 431 | CA | SER | | 71 | −13.527 | 34.319 | −40.064 | 1.00 | 26.65 | C |
| ATOM | 432 | CB | SER | | 71 | −14.913 | 34.848 | −40.446 | 1.00 | 26.56 | C |
| ATOM | 433 | OG | SER | | 71 | −15.368 | 34.265 | −41.654 | 1.00 | 28.18 | O |
| ATOM | 434 | C | SER | | 71 | −12.512 | 34.712 | −41.137 | 1.00 | 25.86 | C |
| ATOM | 435 | O | SER | | 71 | −11.817 | 35.718 | −40.997 | 1.00 | 26.08 | O |
| ATOM | 436 | N | SER | | 72 | −12.422 | 33.912 | −42.197 | 1.00 | 24.98 | N |
| ATOM | 437 | CA | SER | | 72 | −11.503 | 34.198 | −43.302 | 1.00 | 24.88 | C |
| ATOM | 438 | CB | SER | | 72 | −10.059 | 33.875 | −42.913 | 1.00 | 22.69 | C |
| ATOM | 439 | OG | SER | | 72 | −9.833 | 32.478 | −42.976 | 1.00 | 21.77 | O |
| ATOM | 440 | C | SER | | 72 | −11.875 | 33.358 | −44.514 | 1.00 | 24.57 | C |
| ATOM | 441 | O | SER | | 72 | −12.752 | 32.499 | −44.436 | 1.00 | 24.22 | O |
| ATOM | 442 | N | TRP | | 73 | −11.192 | 33.596 | −45.630 | 1.00 | 25.35 | N |
| ATOM | 443 | CA | TRP | | 73 | −11.457 | 32.850 | −46.851 | 1.00 | 25.09 | C |
| ATOM | 444 | CB | TRP | | 73 | −10.597 | 33.377 | −48.012 | 1.00 | 24.73 | C |
| ATOM | 445 | CG | TRP | | 73 | −9.115 | 33.100 | −47.871 | 1.00 | 24.60 | C |
| ATOM | 446 | CD2 | TRP | | 73 | −8.406 | 31.953 | −48.361 | 1.00 | 24.43 | C |
| ATOM | 447 | CE2 | TRP | | 73 | −7.051 | 32.101 | −47.978 | 1.00 | 23.41 | C |
| ATOM | 448 | CE3 | TRP | | 73 | −8.784 | 30.811 | −49.085 | 1.00 | 24.14 | C |
| ATOM | 449 | CD1 | TRP | | 73 | −8.185 | 33.878 | −47.229 | 1.00 | 24.29 | C |
| ATOM | 450 | NE1 | TRP | | 73 | −6.945 | 33.282 | −47.292 | 1.00 | 24.02 | N |
| ATOM | 451 | CZ2 | TRP | | 73 | −6.074 | 31.151 | −48.295 | 1.00 | 23.17 | C |
| ATOM | 452 | CZ3 | TRP | | 73 | −7.806 | 29.862 | −49.401 | 1.00 | 24.96 | C |
| ATOM | 453 | CH2 | TRP | | 73 | −6.468 | 30.042 | −49.003 | 1.00 | 24.47 | C |
| ATOM | 454 | C | TRP | | 73 | −11.183 | 31.360 | −46.658 | 1.00 | 24.64 | C |
| ATOM | 455 | O | TRP | | 73 | −11.684 | 30.535 | −47.417 | 1.00 | 24.77 | O |
| ATOM | 456 | N | ARG | | 74 | −10.391 | 31.017 | −45.642 | 1.00 | 25.31 | N |
| ATOM | 457 | CA | ARG | | 74 | −10.056 | 29.621 | −45.382 | 1.00 | 25.07 | C |
| ATOM | 458 | CB | ARG | | 74 | −8.772 | 29.520 | −44.554 | 1.00 | 25.12 | C |
| ATOM | 459 | CG | ARG | | 74 | −7.568 | 30.213 | −45.172 | 1.00 | 24.30 | C |
| ATOM | 460 | CD | ARG | | 74 | −6.288 | 29.655 | −44.572 | 1.00 | 24.54 | C |
| ATOM | 461 | NE | ARG | | 74 | −6.157 | 28.243 | −44.912 | 1.00 | 26.15 | N |
| ATOM | 462 | CZ | ARG | | 74 | −5.761 | 27.295 | −44.073 | 1.00 | 27.56 | C |
| ATOM | 463 | NH1 | ARG | | 74 | −5.447 | 27.596 | −42.819 | 1.00 | 29.78 | N |
| ATOM | 464 | NH2 | ARG | | 74 | −5.685 | 26.039 | −44.490 | 1.00 | 30.29 | N |
| ATOM | 465 | C | ARG | | 74 | −11.173 | 28.857 | −44.676 | 1.00 | 27.38 | C |
| ATOM | 466 | O | ARG | | 74 | −11.407 | 27.684 | −44.966 | 1.00 | 27.18 | O |
| ATOM | 467 | N | GLN | | 75 | −11.843 | 29.512 | −43.734 | 1.00 | 27.92 | N |
| ATOM | 468 | CA | GLN | | 75 | −12.945 | 28.879 | −43.020 | 1.00 | 29.57 | C |
| ATOM | 469 | CB | GLN | | 75 | −12.431 | 27.971 | −41.896 | 1.00 | 32.69 | C |
| ATOM | 470 | CG | GLN | | 75 | −11.581 | 28.656 | −40.859 | 1.00 | 36.95 | C |
| ATOM | 471 | CD | GLN | | 75 | −11.155 | 27.720 | −39.742 | 1.00 | 38.92 | C |
| ATOM | 472 | OE1 | GLN | | 75 | −10.403 | 28.108 | −38.851 | 1.00 | 40.52 | O |
| ATOM | 473 | NE2 | GLN | | 75 | −11.641 | 26.482 | −39.781 | 1.00 | 40.62 | N |
| ATOM | 474 | C | GLN | | 75 | −13.887 | 29.936 | −42.466 | 1.00 | 28.89 | C |
| ATOM | 475 | O | GLN | | 75 | −13.459 | 30.935 | −41.879 | 1.00 | 27.69 | O |
| ATOM | 476 | N | GLU | | 76 | −15.180 | 29.705 | −42.663 | 1.00 | 27.66 | N |
| ATOM | 477 | CA | GLU | | 76 | −16.202 | 30.639 | −42.231 | 1.00 | 26.40 | C |
| ATOM | 478 | CB | GLU | | 76 | −17.579 | 30.134 | −42.667 | 1.00 | 26.47 | C |
| ATOM | 479 | CG | GLU | | 76 | −18.684 | 31.165 | −42.528 | 1.00 | 26.91 | C |
| ATOM | 480 | CD | GLU | | 76 | −19.934 | 30.790 | −43.315 | 1.00 | 27.18 | C |
| ATOM | 481 | OE1 | GLU | | 76 | −20.872 | 31.614 | −43.372 | 1.00 | 29.82 | O |
| ATOM | 482 | OE2 | GLU | | 76 | −19.976 | 29.675 | −43.877 | 1.00 | 25.98 | O |
| ATOM | 483 | C | GLU | | 76 | −16.198 | 30.916 | −40.734 | 1.00 | 27.16 | C |
| ATOM | 484 | O | GLU | | 76 | −16.338 | 32.065 | −40.317 | 1.00 | 25.65 | O |
| ATOM | 485 | N | LYS | | 77 | −16.044 | 29.870 | −39.931 | 1.00 | 29.05 | N |
| ATOM | 486 | CA | LYS | | 77 | −16.029 | 30.022 | −38.479 | 1.00 | 31.37 | C |
| ATOM | 487 | CB | LYS | | 77 | −17.160 | 29.200 | −37.851 | 1.00 | 33.10 | C |
| ATOM | 488 | CG | LYS | | 77 | −17.319 | 29.402 | −36.351 | 1.00 | 37.18 | C |
| ATOM | 489 | CD | LYS | | 77 | −18.448 | 28.545 | −35.790 | 1.00 | 39.62 | C |
| ATOM | 490 | CE | LYS | | 77 | −18.664 | 28.807 | −34.303 | 1.00 | 41.00 | C |
| ATOM | 491 | NZ | LYS | | 77 | −19.061 | 30.220 | −34.031 | 1.00 | 42.21 | N |
| ATOM | 492 | C | LYS | | 77 | −14.689 | 29.570 | −37.905 | 1.00 | 29.98 | C |
| ATOM | 493 | O | LYS | | 77 | −14.426 | 28.374 | −37.801 | 1.00 | 30.91 | O |
| ATOM | 494 | N | ILE | | 78 | −13.844 | 30.530 | −37.539 | 1.00 | 29.93 | N |
| ATOM | 495 | CA | ILE | | 78 | −12.534 | 30.218 | −36.967 | 1.00 | 29.28 | C |
| ATOM | 496 | CB | ILE | | 78 | −11.571 | 31.416 | −37.076 | 1.00 | 29.22 | C |
| ATOM | 497 | CG2 | ILE | | 78 | −10.222 | 31.048 | −36.462 | 1.00 | 27.44 | C |
| ATOM | 498 | CG1 | ILE | | 78 | −11.429 | 31.855 | −38.539 | 1.00 | 30.95 | C |
| ATOM | 499 | CD1 | ILE | | 78 | −10.372 | 31.116 | −39.325 | 1.00 | 30.51 | C |
| ATOM | 500 | C | ILE | | 78 | −12.728 | 29.906 | −35.487 | 1.00 | 28.73 | C |
| ATOM | 501 | O | ILE | | 78 | −13.116 | 30.782 | −34.715 | 1.00 | 29.60 | O |
| ATOM | 502 | N | THR | | 79 | −12.443 | 28.670 | −35.091 | 1.00 | 28.73 | N |
| ATOM | 503 | CA | THR | | 79 | −12.621 | 28.270 | −33.701 | 1.00 | 29.99 | C |
| ATOM | 504 | CB | THR | | 79 | −13.601 | 27.097 | −33.607 | 1.00 | 30.94 | C |
| ATOM | 505 | OG1 | THR | | 79 | −13.111 | 26.005 | −34.393 | 1.00 | 33.62 | O |
| ATOM | 506 | CG2 | THR | | 79 | −14.967 | 27.510 | −34.121 | 1.00 | 32.21 | C |
| ATOM | 507 | C | THR | | 79 | −11.346 | 27.882 | −32.948 | 1.00 | 30.42 | C |
| ATOM | 508 | O | THR | | 79 | −11.389 | 27.645 | −31.737 | 1.00 | 30.20 | O |
| ATOM | 509 | N | ARG | | 80 | −10.216 | 27.809 | −33.648 | 1.00 | 29.26 | N |
| ATOM | 510 | CA | ARG | | 80 | −8.969 | 27.438 | −32.985 | 1.00 | 27.73 | C |
| ATOM | 511 | CB | ARG | | 80 | −7.810 | 27.389 | −33.996 | 1.00 | 27.79 | C |
| ATOM | 512 | CG | ARG | | 80 | −7.412 | 28.737 | −34.595 | 1.00 | 26.61 | C |
| ATOM | 513 | CD | ARG | | 80 | −6.092 | 28.621 | −35.361 | 1.00 | 23.85 | C |
| ATOM | 514 | NE | ARG | | 80 | −6.234 | 27.895 | −36.621 | 1.00 | 23.38 | N |
| ATOM | 515 | CZ | ARG | | 80 | −6.593 | 28.454 | −37.775 | 1.00 | 21.95 | C |
| ATOM | 516 | NH1 | ARG | | 80 | −6.846 | 29.755 | −37.834 | 1.00 | 22.12 | N |
| ATOM | 517 | NH2 | ARG | | 80 | −6.697 | 27.712 | −38.873 | 1.00 | 22.26 | N |
| ATOM | 518 | C | ARG | | 80 | −8.653 | 28.423 | −31.863 | 1.00 | 26.29 | C |
| ATOM | 519 | O | ARG | | 80 | −9.010 | 29.598 | −31.936 | 1.00 | 27.24 | O |
| ATOM | 520 | N | THR | | 81 | −7.997 | 27.943 | −30.811 | 1.00 | 26.49 | N |
| ATOM | 521 | CA | THR | | 81 | −7.650 | 28.810 | −29.694 | 1.00 | 26.30 | C |
| ATOM | 522 | CB | THR | | 81 | −7.370 | 27.999 | −28.413 | 1.00 | 27.36 | C |
| ATOM | 523 | OG1 | THR | | 81 | −6.162 | 27.244 | −28.572 | 1.00 | 27.30 | O |
| ATOM | 524 | CG2 | THR | | 81 | −8.526 | 27.039 | −28.131 | 1.00 | 28.20 | C |
| ATOM | 525 | C | THR | | 81 | −6.407 | 29.623 | −30.025 | 1.00 | 26.40 | C |
| ATOM | 526 | O | THR | | 81 | −5.664 | 29.288 | −30.946 | 1.00 | 24.80 | O |
| ATOM | 527 | N | LYS | | 82 | −6.193 | 30.695 | −29.273 | 1.00 | 26.41 | N |
| ATOM | 528 | CA | LYS | | 82 | −5.028 | 31.547 | −29.468 | 1.00 | 27.53 | C |
| ATOM | 529 | CB | LYS | | 82 | −5.030 | 32.666 | −28.425 | 1.00 | 30.37 | C |
| ATOM | 530 | CG | LYS | | 82 | −3.857 | 33.630 | −28.512 | 1.00 | 35.57 | C |
| ATOM | 531 | CD | LYS | | 82 | −4.015 | 34.773 | −27.511 | 1.00 | 38.91 | C |
| ATOM | 532 | CE | LYS | | 82 | −4.170 | 34.248 | −26.088 | 1.00 | 42.86 | C |
| ATOM | 533 | NZ | LYS | | 82 | −4.295 | 35.344 | −25.089 | 1.00 | 44.49 | N |
| ATOM | 534 | C | LYS | | 82 | −3.768 | 30.693 | −29.327 | 1.00 | 26.88 | C |
| ATOM | 535 | O | LYS | | 82 | −2.804 | 30.846 | −30.077 | 1.00 | 24.49 | O |
| ATOM | 536 | N | GLU | | 83 | −3.788 | 29.780 | −28.364 | 1.00 | 25.38 | N |
| ATOM | 537 | CA | GLU | | 83 | −2.651 | 28.903 | −28.129 | 1.00 | 25.18 | C |
| ATOM | 538 | CB | GLU | | 83 | −2.901 | 28.040 | −26.887 | 1.00 | 27.21 | C |
| ATOM | 539 | CG | GLU | | 83 | −2.947 | 28.836 | −25.586 | 1.00 | 31.61 | C |
| ATOM | 540 | CD | GLU | | 83 | −4.190 | 29.708 | −25.444 | 1.00 | 34.31 | C |
| ATOM | 541 | OE1 | GLU | | 83 | −4.123 | 30.713 | −24.702 | 1.00 | 37.68 | O |
| ATOM | 542 | OE2 | GLU | | 83 | −5.235 | 29.387 | −26.054 | 1.00 | 33.33 | O |
| ATOM | 543 | C | GLU | | 83 | −2.378 | 28.015 | −29.339 | 1.00 | 23.21 | C |
| ATOM | 544 | O | GLU | | 83 | −1.222 | 27.786 | −29.699 | 1.00 | 21.40 | O |
| ATOM | 545 | N | GLU | | 84 | −3.439 | 27.521 | −29.973 | 1.00 | 22.12 | N |
| ATOM | 546 | CA | GLU | | 84 | −3.292 | 26.661 | −31.146 | 1.00 | 22.54 | C |
| ATOM | 547 | CB | GLU | | 84 | −4.626 | 25.993 | −31.500 | 1.00 | 24.78 | C |
| ATOM | 548 | CG | GLU | | 84 | −5.163 | 25.062 | −30.422 | 1.00 | 29.89 | C |
| ATOM | 549 | CD | GLU | | 84 | −6.548 | 24.531 | −30.749 | 1.00 | 31.60 | C |
| ATOM | 550 | OE1 | GLU | | 84 | −7.454 | 25.355 | −30.994 | 1.00 | 30.86 | O |
| ATOM | 551 | OE2 | GLU | | 84 | −6.731 | 23.295 | −30.759 | 1.00 | 33.98 | O |
| ATOM | 552 | C | GLU | | 84 | −2.799 | 27.487 | −32.333 | 1.00 | 21.41 | C |
| ATOM | 553 | O | GLU | | 84 | −2.035 | 27.000 | −33.170 | 1.00 | 20.63 | O |
| ATOM | 554 | N | ALA | | 85 | −3.242 | 28.736 | −32.404 | 1.00 | 18.73 | N |
| ATOM | 555 | CA | ALA | | 85 | −2.813 | 29.622 | −33.478 | 1.00 | 19.58 | C |
| ATOM | 556 | CB | ALA | | 85 | −3.558 | 30.950 | −33.393 | 1.00 | 19.88 | C |
| ATOM | 557 | C | ALA | | 85 | −1.304 | 29.859 | −33.360 | 1.00 | 20.03 | C |
| ATOM | 558 | O | ALA | | 85 | −0.582 | 29.842 | −34.364 | 1.00 | 17.91 | O |
| ATOM | 559 | N | LEU | | 86 | −0.826 | 30.067 | −32.135 | 1.00 | 18.83 | N |
| ATOM | 560 | CA | LEU | | 86 | 0.599 | 30.309 | −31.924 | 1.00 | 18.94 | C |
| ATOM | 561 | CB | LEU | | 86 | 0.877 | 30.675 | −30.457 | 1.00 | 17.95 | C |
| ATOM | 562 | CG | LEU | | 86 | 2.325 | 31.013 | −30.069 | 1.00 | 20.47 | C |
| ATOM | 563 | CD1 | LEU | | 86 | 2.874 | 32.114 | −30.970 | 1.00 | 19.07 | C |
| ATOM | 564 | CD2 | LEU | | 86 | 2.373 | 31.465 | −28.612 | 1.00 | 20.51 | C |
| ATOM | 565 | C | LEU | | 86 | 1.436 | 29.101 | −32.338 | 1.00 | 19.08 | C |
| ATOM | 566 | O | LEU | | 86 | 2.513 | 29.254 | −32.913 | 1.00 | 18.44 | O |
| ATOM | 567 | N | GLU | | 87 | 0.953 | 27.895 | −32.061 | 1.00 | 19.94 | N |
| ATOM | 568 | CA | GLU | | 87 | 1.715 | 26.712 | −32.445 | 1.00 | 22.10 | C |
| ATOM | 569 | CB | GLU | | 87 | 1.104 | 25.455 | −31.829 | 1.00 | 25.88 | C |
| ATOM | 570 | CG | GLU | | 87 | 1.394 | 25.378 | −30.343 | 1.00 | 33.91 | C |
| ATOM | 571 | CD | GLU | | 87 | 2.850 | 25.708 | −30.043 | 1.00 | 39.69 | C |
| ATOM | 572 | OE1 | GLU | | 87 | 3.737 | 24.957 | −30.506 | 1.00 | 41.93 | O |
| ATOM | 573 | OE2 | GLU | | 87 | 3.109 | 26.726 | −29.359 | 1.00 | 43.20 | O |
| ATOM | 574 | C | GLU | | 87 | 1.833 | 26.572 | −33.953 | 1.00 | 20.94 | C |
| ATOM | 575 | O | GLU | | 87 | 2.866 | 26.130 | −34.464 | 1.00 | 19.53 | O |
| ATOM | 576 | N | LEU | | 88 | 0.781 | 26.953 | −34.669 | 1.00 | 19.04 | N |
| ATOM | 577 | CA | LEU | | 88 | 0.797 | 26.894 | −36.123 | 1.00 | 18.20 | C |
| ATOM | 578 | CB | LEU | | 88 | −0.594 | 27.213 | −36.678 | 1.00 | 17.83 | C |
| ATOM | 579 | CG | LEU | | 88 | −1.607 | 26.074 | −36.543 | 1.00 | 21.08 | C |
| ATOM | 580 | CD1 | LEU | | 88 | −3.026 | 26.594 | −36.738 | 1.00 | 20.47 | C |
| ATOM | 581 | CD2 | LEU | | 88 | −1.276 | 24.994 | −37.563 | 1.00 | 21.11 | C |
| ATOM | 582 | C | LEU | | 88 | 1.813 | 27.915 | −36.629 | 1.00 | 16.72 | C |
| ATOM | 583 | O | LEU | | 88 | 2.625 | 27.618 | −37.507 | 1.00 | 17.60 | O |
| ATOM | 584 | N | ILE | | 89 | 1.759 | 29.119 | −36.068 | 1.00 | 16.37 | N |
| ATOM | 585 | CA | ILE | | 89 | 2.688 | 30.185 | −36.437 | 1.00 | 16.70 | C |
| ATOM | 586 | CB | ILE | | 89 | 2.398 | 31.471 | −35.619 | 1.00 | 17.17 | C |
| ATOM | 587 | CG2 | ILE | | 89 | 3.599 | 32.405 | −35.637 | 1.00 | 15.46 | C |
| ATOM | 588 | CG1 | ILE | | 89 | 1.155 | 32.179 | −36.174 | 1.00 | 16.85 | C |
| ATOM | 589 | CD1 | ILE | | 89 | 1.404 | 33.010 | −37.442 | 1.00 | 14.20 | C |
| ATOM | 590 | C | ILE | | 89 | 4.121 | 29.712 | −36.174 | 1.00 | 17.79 | C |
| ATOM | 591 | O | ILE | | 89 | 5.002 | 29.863 | −37.026 | 1.00 | 14.51 | O |
| ATOM | 592 | N | ASN | | 90 | 4.356 | 29.129 | −35.002 | 1.00 | 17.52 | N |
| ATOM | 593 | CA | ASN | | 90 | 5.700 | 28.657 | −34.686 | 1.00 | 18.01 | C |
| ATOM | 594 | CB | ASN | | 90 | 5.776 | 28.143 | −33.244 | 1.00 | 19.64 | C |
| ATOM | 595 | CG | ASN | | 90 | 5.750 | 29.264 | −32.223 | 1.00 | 22.39 | C |
| ATOM | 596 | OD1 | ASN | | 90 | 6.215 | 30.374 | −32.487 | 1.00 | 24.53 | O |
| ATOM | 597 | ND2 | ASN | | 90 | 5.227 | 28.974 | −31.040 | 1.00 | 25.12 | N |
| ATOM | 598 | C | ASN | | 90 | 6.158 | 27.577 | −35.660 | 1.00 | 17.58 | C |
| ATOM | 599 | O | ASN | | 90 | 7.330 | 27.528 | −36.029 | 1.00 | 16.24 | O |
| ATOM | 600 | N | GLY | | 91 | 5.231 | 26.725 | −36.090 | 1.00 | 17.69 | N |
| ATOM | 601 | CA | GLY | | 91 | 5.575 | 25.675 | −37.032 | 1.00 | 16.76 | C |
| ATOM | 602 | C | GLY | | 91 | 5.965 | 26.258 | −38.379 | 1.00 | 17.49 | C |
| ATOM | 603 | O | GLY | | 91 | 6.913 | 25.802 | −39.023 | 1.00 | 16.71 | O |
| ATOM | 604 | N | TYR | | 92 | 5.228 | 27.274 | −38.818 | 1.00 | 15.08 | N |
| ATOM | 605 | CA | TYR | | 92 | 5.523 | 27.922 | −40.096 | 1.00 | 15.07 | C |
| ATOM | 606 | CB | TYR | | 92 | 4.456 | 28.978 | −40.419 | 1.00 | 14.21 | C |
| ATOM | 607 | CG | TYR | | 92 | 3.069 | 28.410 | −40.637 | 1.00 | 16.80 | C |
| ATOM | 608 | CD1 | TYR | | 92 | 1.933 | 29.150 | −40.306 | 1.00 | 17.92 | C |
| ATOM | 609 | CE1 | TYR | | 92 | 0.653 | 28.633 | −40.502 | 1.00 | 18.52 | C |
| ATOM | 610 | CD2 | TYR | | 92 | 2.890 | 27.136 | −41.178 | 1.00 | 17.96 | C |
| ATOM | 611 | CE2 | TYR | | 92 | 1.615 | 26.610 | −41.379 | 1.00 | 18.48 | C |
| ATOM | 612 | CZ | TYR | | 92 | 0.503 | 27.365 | −41.037 | 1.00 | 19.52 | C |
| ATOM | 613 | OH | TYR | | 92 | −0.762 | 26.848 | −41.220 | 1.00 | 18.16 | O |
| ATOM | 614 | C | TYR | | 92 | 6.890 | 28.592 | −40.036 | 1.00 | 13.58 | C |
| ATOM | 615 | O | TYR | | 92 | 7.672 | 28.521 | −40.985 | 1.00 | 14.84 | O |
| ATOM | 616 | N | ILE | | 93 | 7.182 | 29.242 | −38.919 | 1.00 | 13.07 | N |
| ATOM | 617 | CA | ILE | | 93 | 8.467 | 29.912 | −38.774 | 1.00 | 12.46 | C |
| ATOM | 618 | CB | ILE | | 93 | 8.555 | 30.685 | −37.429 | 1.00 | 11.79 | C |
| ATOM | 619 | CG2 | ILE | | 93 | 9.997 | 31.142 | −37.171 | 1.00 | 14.42 | C |
| ATOM | 620 | CG1 | ILE | | 93 | 7.622 | 31.907 | −37.482 | 1.00 | 13.71 | C |
| ATOM | 621 | CD1 | ILE | | 93 | 7.620 | 32.762 | −36.207 | 1.00 | 14.39 | C |
| ATOM | 622 | C | ILE | | 93 | 9.601 | 28.900 | −38.879 | 1.00 | 14.34 | C |
| ATOM | 623 | O | ILE | | 93 | 10.611 | 29.156 | −39.544 | 1.00 | 11.30 | O |
| ATOM | 624 | N | GLN | | 94 | 9.426 | 27.743 | −38.247 | 1.00 | 14.83 | N |
| ATOM | 625 | CA | GLN | | 94 | 10.452 | 26.709 | −38.293 | 1.00 | 17.56 | C |
| ATOM | 626 | CB | GLN | | 94 | 10.071 | 25.510 | −37.413 | 1.00 | 18.87 | C |
| ATOM | 627 | CG | GLN | | 94 | 10.163 | 25.790 | −35.922 | 1.00 | 22.84 | C |
| ATOM | 628 | CD | GLN | | 94 | 9.911 | 24.556 | −35.079 | 1.00 | 28.77 | C |
| ATOM | 629 | OE1 | GLN | | 94 | 8.900 | 23.872 | −35.242 | 1.00 | 29.97 | O |
| ATOM | 630 | NE2 | GLN | | 94 | 10.831 | 24.266 | −34.166 | 1.00 | 31.41 | N |
| ATOM | 631 | C | GLN | | 94 | 10.695 | 26.238 | −39.714 | 1.00 | 16.92 | C |
| ATOM | 632 | O | GLN | | 94 | 11.841 | 26.064 | −40.118 | 1.00 | 18.90 | O |
| ATOM | 633 | N | LYS | | 95 | 9.623 | 26.038 | −40.476 | 1.00 | 17.46 | N |
| ATOM | 634 | CA | LYS | | 95 | 9.766 | 25.581 | −41.850 | 1.00 | 17.51 | C |
| ATOM | 635 | CB | LYS | | 95 | 8.409 | 25.152 | −42.415 | 1.00 | 21.44 | C |
| ATOM | 636 | CG | LYS | | 95 | 7.724 | 24.060 | −41.607 | 1.00 | 27.33 | C |
| ATOM | 637 | CD | LYS | | 95 | 6.521 | 23.491 | −42.347 | 1.00 | 31.14 | C |
| ATOM | 638 | CE | LYS | | 95 | 5.590 | 22.710 | −41.412 | 1.00 | 35.50 | C |
| ATOM | 639 | NZ | LYS | | 95 | 4.796 | 23.608 | −40.505 | 1.00 | 33.20 | N |
| ATOM | 640 | C | LYS | | 95 | 10.384 | 26.658 | −42.739 | 1.00 | 16.47 | C |
| ATOM | 641 | O | LYS | | 95 | 11.117 | 26.356 | −43.682 | 1.00 | 17.74 | O |
| ATOM | 642 | N | ILE | | 96 | 10.088 | 27.917 | −42.449 | 1.00 | 15.01 | N |
| ATOM | 643 | CA | ILE | | 96 | 10.647 | 28.998 | −43.242 | 1.00 | 13.96 | C |
| ATOM | 644 | CB | ILE | | 96 | 9.930 | 30.338 | −42.939 | 1.00 | 14.06 | C |
| ATOM | 645 | CG2 | ILE | | 96 | 10.645 | 31.492 | −43.639 | 1.00 | 13.12 | C |
| ATOM | 646 | CG1 | ILE | | 96 | 8.476 | 30.253 | −43.420 | 1.00 | 13.74 | C |
| ATOM | 647 | CD1 | ILE | | 96 | 7.593 | 31.420 | −42.981 | 1.00 | 17.05 | C |
| ATOM | 648 | C | ILE | | 96 | 12.143 | 29.125 | −42.954 | 1.00 | 12.71 | C |
| ATOM | 649 | O | ILE | | 96 | 12.948 | 29.258 | −43.873 | 1.00 | 13.62 | O |
| ATOM | 650 | N | LYS | | 97 | 12.517 | 29.058 | −41.682 | 1.00 | 13.77 | N |
| ATOM | 651 | CA | LYS | | 97 | 13.926 | 29.187 | −41.314 | 1.00 | 14.47 | C |
| ATOM | 652 | CB | LYS | | 97 | 14.078 | 29.356 | −39.796 | 1.00 | 13.38 | C |
| ATOM | 653 | CG | LYS | | 97 | 13.716 | 30.752 | −39.291 | 1.00 | 16.41 | C |
| ATOM | 654 | CD | LYS | | 97 | 14.159 | 30.948 | −37.846 | 1.00 | 15.94 | C |
| ATOM | 655 | CE | LYS | | 97 | 14.098 | 32.413 | −37.437 | 1.00 | 16.64 | C |
| ATOM | 656 | NZ | LYS | | 97 | 14.566 | 32.624 | −36.035 | 1.00 | 14.12 | N |
| ATOM | 657 | C | LYS | | 97 | 14.785 | 28.023 | −41.794 | 1.00 | 16.02 | C |
| ATOM | 658 | O | LYS | | 97 | 15.965 | 28.210 | −42.097 | 1.00 | 16.53 | O |
| ATOM | 659 | N | SER | | 98 | 14.203 | 26.829 | −41.869 | 1.00 | 15.75 | N |
| ATOM | 660 | CA | SER | | 98 | 14.949 | 25.652 | −42.331 | 1.00 | 17.11 | C |
| ATOM | 661 | CB | SER | | 98 | 14.266 | 24.369 | −41.856 | 1.00 | 17.57 | C |
| ATOM | 662 | OG | SER | | 98 | 12.992 | 24.222 | −42.460 | 1.00 | 17.95 | O |
| ATOM | 663 | C | SER | | 98 | 15.065 | 25.616 | −43.855 | 1.00 | 17.15 | C |
| ATOM | 664 | O | SER | | 98 | 15.914 | 24.916 | −44.413 | 1.00 | 17.05 | O |
| ATOM | 665 | N | GLY | | 99 | 14.206 | 26.369 | −44.530 | 1.00 | 16.27 | N |
| ATOM | 666 | CA | GLY | | 99 | 14.238 | 26.381 | −45.978 | 1.00 | 18.16 | C |
| ATOM | 667 | C | GLY | | 99 | 13.358 | 25.298 | −46.573 | 1.00 | 19.26 | C |
| ATOM | 668 | O | GLY | | 99 | 13.287 | 25.158 | −47.792 | 1.00 | 20.98 | O |
| ATOM | 669 | N | GLU | | 100 | 12.684 | 24.533 | −45.718 | 1.00 | 19.28 | N |
| ATOM | 670 | CA | GLU | | 100 | 11.802 | 23.465 | −46.179 | 1.00 | 22.63 | C |
| ATOM | 671 | CB | GLU | | 100 | 11.232 | 22.691 | −44.989 | 1.00 | 24.56 | C |
| ATOM | 672 | CG | GLU | | 100 | 12.282 | 22.082 | −44.080 | 1.00 | 34.69 | C |
| ATOM | 673 | CD | GLU | | 100 | 11.709 | 21.629 | −42.748 | 1.00 | 38.50 | C |
| ATOM | 674 | OE1 | GLU | | 100 | 12.500 | 21.310 | −41.836 | 1.00 | 42.22 | O |
| ATOM | 675 | OE2 | GLU | | 100 | 10.468 | 21.589 | −42.611 | 1.00 | 43.33 | O |
| ATOM | 676 | C | GLU | | 100 | 10.657 | 24.071 | −46.976 | 1.00 | 23.47 | C |
| ATOM | 677 | O | GLU | | 100 | 10.177 | 23.482 | −47.941 | 1.00 | 24.10 | O |
| ATOM | 678 | N | GLU | | 101 | 10.227 | 25.259 | −46.559 | 1.00 | 22.54 | N |
| ATOM | 679 | CA | GLU | | 101 | 9.133 | 25.973 | −47.211 | 1.00 | 25.17 | C |
| ATOM | 680 | CB | GLU | | 101 | 7.845 | 25.804 | −46.400 | 1.00 | 27.97 | C |
| ATOM | 681 | CG | GLU | | 101 | 7.207 | 24.445 | −46.523 | 1.00 | 32.58 | C |
| ATOM | 682 | CD | GLU | | 101 | 6.336 | 24.348 | −47.750 | 1.00 | 34.33 | C |
| ATOM | 683 | OE1 | GLU | | 101 | 6.783 | 24.781 | −48.833 | 1.00 | 38.51 | O |
| ATOM | 684 | OE2 | GLU | | 101 | 5.203 | 23.840 | −47.629 | 1.00 | 37.40 | O |
| ATOM | 685 | C | GLU | | 101 | 9.466 | 27.450 | −47.267 | 1.00 | 24.41 | C |
| ATOM | 686 | O | GLU | | 101 | 10.126 | 27.962 | −46.368 | 1.00 | 25.29 | O |
| ATOM | 687 | N | ASP | | 102 | 9.037 | 28.149 | −48.311 | 1.00 | 21.76 | N |
| ATOM | 688 | CA | ASP | | 102 | 9.317 | 29.569 | −48.312 | 1.00 | 23.85 | C |
| ATOM | 689 | CB | ASP | | 102 | 9.811 | 30.069 | −49.681 | 1.00 | 31.68 | C |
| ATOM | 690 | CG | ASP | | 102 | 8.905 | 29.693 | −50.818 | 1.00 | 36.62 | C |
| ATOM | 691 | OD1 | ASP | | 102 | 9.416 | 29.626 | −51.962 | 1.00 | 40.04 | O |
| ATOM | 692 | OD2 | ASP | | 102 | 7.698 | 29.481 | −50.585 | 1.00 | 40.03 | O |
| ATOM | 693 | C | ASP | | 102 | 8.096 | 30.333 | −47.821 | 1.00 | 20.96 | C |
| ATOM | 694 | O | ASP | | 102 | 6.960 | 29.851 | −47.887 | 1.00 | 18.92 | O |
| ATOM | 695 | N | PHE | | 103 | 8.354 | 31.507 | −47.272 | 1.00 | 17.56 | N |
| ATOM | 696 | CA | PHE | | 103 | 7.308 | 32.356 | −46.728 | 1.00 | 17.86 | C |
| ATOM | 697 | CB | PHE | | 103 | 7.915 | 33.715 | −46.359 | 1.00 | 17.46 | C |
| ATOM | 698 | CG | PHE | | 103 | 6.910 | 34.720 | −45.896 | 1.00 | 19.96 | C |
| ATOM | 699 | CD1 | PHE | | 103 | 6.575 | 34.821 | −44.552 | 1.00 | 22.44 | C |
| ATOM | 700 | CD2 | PHE | | 103 | 6.242 | 35.521 | −46.819 | 1.00 | 22.86 | C |
| ATOM | 701 | CE1 | PHE | | 103 | 5.586 | 35.698 | −44.131 | 1.00 | 21.59 | C |
| ATOM | 702 | CE2 | PHE | | 103 | 5.247 | 36.403 | −46.404 | 1.00 | 23.10 | C |
| ATOM | 703 | CZ | PHE | | 103 | 4.921 | 36.487 | −45.058 | 1.00 | 23.34 | C |
| ATOM | 704 | C | PHE | | 103 | 6.128 | 32.551 | −47.684 | 1.00 | 16.10 | C |
| ATOM | 705 | O | PHE | | 103 | 4.964 | 32.406 | −47.296 | 1.00 | 15.17 | O |
| ATOM | 706 | N | GLU | | 104 | 6.433 | 32.878 | −48.935 | 1.00 | 16.13 | N |
| ATOM | 707 | CA | GLU | | 104 | 5.401 | 33.128 | −49.934 | 1.00 | 17.66 | C |
| ATOM | 708 | CB | GLU | | 104 | 6.046 | 33.514 | −51.270 | 1.00 | 17.40 | C |
| ATOM | 709 | CG | GLU | | 104 | 6.840 | 34.803 | −51.203 | 1.00 | 18.94 | C |
| ATOM | 710 | CD | GLU | | 104 | 8.293 | 34.596 | −50.804 | 1.00 | 20.69 | C |
| ATOM | 711 | OE1 | GLU | | 104 | 8.616 | 33.564 | −50.177 | 1.00 | 19.24 | O |
| ATOM | 712 | OE2 | GLU | | 104 | 9.114 | 35.484 | −51.114 | 1.00 | 23.94 | O |
| ATOM | 713 | C | GLU | | 104 | 4.438 | 31.971 | −50.145 | 1.00 | 17.68 | C |
| ATOM | 714 | O | GLU | | 104 | 3.228 | 32.183 | −50.311 | 1.00 | 18.14 | O |
| ATOM | 715 | N | SER | | 105 | 4.969 | 30.754 | −50.142 | 1.00 | 17.65 | N |
| ATOM | 716 | CA | SER | | 105 | 4.144 | 29.570 | −50.339 | 1.00 | 19.62 | C |
| ATOM | 717 | CB | SER | | 105 | 5.025 | 28.342 | −50.557 | 1.00 | 21.10 | C |
| ATOM | 718 | OG | SER | | 105 | 4.242 | 27.167 | −50.592 | 1.00 | 24.46 | O |
| ATOM | 719 | C | SER | | 105 | 3.210 | 29.330 | −49.154 | 1.00 | 17.87 | C |
| ATOM | 720 | O | SER | | 105 | 2.032 | 29.031 | −49.339 | 1.00 | 16.82 | O |
| ATOM | 721 | N | LEU | | 106 | 3.735 | 29.457 | −47.939 | 1.00 | 17.50 | N |
| ATOM | 722 | CA | LEU | | 106 | 2.915 | 29.252 | −46.745 | 1.00 | 15.45 | C |
| ATOM | 723 | CB | LEU | | 106 | 3.799 | 29.167 | −45.491 | 1.00 | 14.63 | C |
| ATOM | 724 | CG | LEU | | 106 | 4.794 | 27.998 | −45.438 | 1.00 | 14.52 | C |
| ATOM | 725 | CD1 | LEU | | 106 | 5.587 | 28.048 | −44.129 | 1.00 | 13.75 | C |
| ATOM | 726 | CD2 | LEU | | 106 | 4.055 | 26.679 | −45.545 | 1.00 | 15.48 | C |
| ATOM | 727 | C | LEU | | 106 | 1.881 | 30.370 | −46.594 | 1.00 | 15.13 | C |
| ATOM | 728 | O | LEU | | 106 | 0.766 | 30.126 | −46.130 | 1.00 | 17.62 | O |
| ATOM | 729 | N | ALA | | 107 | 2.236 | 31.592 | −46.986 | 1.00 | 15.69 | N |
| ATOM | 730 | CA | ALA | | 107 | 1.288 | 32.696 | −46.890 | 1.00 | 16.19 | C |
| ATOM | 731 | CB | ALA | | 107 | 1.967 | 34.019 | −47.232 | 1.00 | 16.48 | C |
| ATOM | 732 | C | ALA | | 107 | 0.120 | 32.440 | −47.846 | 1.00 | 17.33 | C |
| ATOM | 733 | O | ALA | | 107 | −1.048 | 32.550 | −47.457 | 1.00 | 17.01 | O |
| ATOM | 734 | N | SER | | 108 | 0.436 | 32.093 | −49.091 | 1.00 | 15.91 | N |
| ATOM | 735 | CA | SER | | 108 | −0.595 | 31.823 | −50.092 | 1.00 | 17.16 | C |
| ATOM | 736 | CB | SER | | 108 | 0.048 | 31.413 | −51.420 | 1.00 | 16.68 | C |
| ATOM | 737 | OG | SER | | 108 | 0.836 | 32.474 | −51.937 | 1.00 | 21.71 | O |
| ATOM | 738 | C | SER | | 108 | −1.543 | 30.722 | −49.642 | 1.00 | 18.11 | C |
| ATOM | 739 | O | SER | | 108 | −2.754 | 30.792 | −49.857 | 1.00 | 18.16 | O |
| ATOM | 740 | N | GLN | | 109 | −0.982 | 29.705 | −49.004 | 1.00 | 17.09 | N |
| ATOM | 741 | CA | GLN | | 109 | −1.762 | 28.569 | −48.550 | 1.00 | 19.26 | C |
| ATOM | 742 | CB | GLN | | 109 | −0.863 | 27.341 | −48.421 | 1.00 | 18.59 | C |
| ATOM | 743 | CG | GLN | | 109 | −0.352 | 26.765 | −49.720 | 1.00 | 19.36 | C |
| ATOM | 744 | CD | GLN | | 109 | 0.631 | 25.644 | −49.472 | 1.00 | 22.99 | C |
| ATOM | 745 | OE1 | GLN | | 109 | 1.843 | 25.866 | −49.372 | 1.00 | 24.27 | O |
| ATOM | 746 | NE2 | GLN | | 109 | 0.114 | 24.431 | −49.345 | 1.00 | 21.44 | N |
| ATOM | 747 | C | GLN | | 109 | −2.506 | 28.712 | −47.236 | 1.00 | 19.19 | C |
| ATOM | 748 | O | GLN | | 109 | −3.657 | 28.294 | −47.125 | 1.00 | 19.84 | O |
| ATOM | 749 | N | PHE | | 110 | −1.859 | 29.309 | −46.244 | 1.00 | 18.50 | N |
| ATOM | 750 | CA | PHE | | 110 | −2.459 | 29.366 | −44.923 | 1.00 | 18.43 | C |
| ATOM | 751 | CB | PHE | | 110 | −1.590 | 28.535 | −43.979 | 1.00 | 18.75 | C |
| ATOM | 752 | CG | PHE | | 110 | −1.240 | 27.185 | −44.526 | 1.00 | 19.15 | C |
| ATOM | 753 | CD1 | PHE | | 110 | 0.075 | 26.869 | −44.846 | 1.00 | 19.29 | C |
| ATOM | 754 | CD2 | PHE | | 110 | −2.228 | 26.233 | −44.743 | 1.00 | 19.69 | C |
| ATOM | 755 | CE1 | PHE | | 110 | 0.401 | 25.623 | −45.373 | 1.00 | 21.20 | C |
| ATOM | 756 | CE2 | PHE | | 110 | −1.913 | 24.985 | −45.271 | 1.00 | 21.37 | C |
| ATOM | 757 | CZ | PHE | | 110 | −0.594 | 24.679 | −45.587 | 1.00 | 20.98 | C |
| ATOM | 758 | C | PHE | | 110 | −2.781 | 30.686 | −44.253 | 1.00 | 17.22 | C |
| ATOM | 759 | O | PHE | | 110 | −3.269 | 30.682 | −43.125 | 1.00 | 19.14 | O |
| ATOM | 760 | N | SER | | 111 | −2.522 | 31.810 | −44.904 | 1.00 | 16.53 | N |
| ATOM | 761 | CA | SER | | 111 | −2.827 | 33.083 | −44.267 | 1.00 | 17.57 | C |
| ATOM | 762 | CB | SER | | 111 | −2.141 | 34.233 | −44.992 | 1.00 | 14.46 | C |
| ATOM | 763 | OG | SER | | 111 | −2.384 | 35.459 | −44.316 | 1.00 | 15.16 | O |
| ATOM | 764 | C | SER | | 111 | −4.336 | 33.329 | −44.241 | 1.00 | 19.62 | C |
| ATOM | 765 | O | SER | | 111 | −5.016 | 33.178 | −45.262 | 1.00 | 18.52 | O |
| ATOM | 766 | N | ASP | | 112 | −4.845 | 33.703 | −43.069 | 1.00 | 19.75 | N |
| ATOM | 767 | CA | ASP | | 112 | −6.270 | 33.981 | −42.881 | 1.00 | 21.92 | C |
| ATOM | 768 | CB | ASP | | 112 | −6.675 | 33.749 | −41.418 | 1.00 | 20.75 | C |
| ATOM | 769 | CG | ASP | | 112 | −6.872 | 32.285 | −41.091 | 1.00 | 22.18 | C |
| ATOM | 770 | OD1 | ASP | | 112 | −7.569 | 31.594 | −41.865 | 1.00 | 22.37 | O |
| ATOM | 771 | OD2 | ASP | | 112 | −6.343 | 31.822 | −40.054 | 1.00 | 21.73 | O |
| ATOM | 772 | C | ASP | | 112 | −6.623 | 35.412 | −43.268 | 1.00 | 21.78 | C |
| ATOM | 773 | O | ASP | | 112 | −7.430 | 36.062 | −42.603 | 1.00 | 22.89 | O |
| ATOM | 774 | N | CYS | | 113 | −6.009 | 35.898 | −44.341 | 1.00 | 22.79 | N |
| ATOM | 775 | CA | CYS | | 113 | −6.242 | 37.249 | −44.830 | 1.00 | 22.62 | C |
| ATOM | 776 | CB | CYS | | 113 | −5.086 | 38.163 | −44.405 | 1.00 | 23.11 | C |
| ATOM | 777 | SG | CYS | | 113 | −5.361 | 39.937 | −44.656 | 1.00 | 29.65 | S |
| ATOM | 778 | C | CYS | | 113 | −6.323 | 37.167 | −46.352 | 1.00 | 22.79 | C |
| ATOM | 779 | O | CYS | | 113 | −5.710 | 36.289 | −46.959 | 1.00 | 21.38 | O |
| ATOM | 780 | N | SER | | 114 | −7.076 | 38.073 | −46.969 | 1.00 | 21.07 | N |
| ATOM | 781 | CA | SER | | 114 | −7.223 | 38.060 | −48.421 | 1.00 | 21.83 | C |
| ATOM | 782 | CB | SER | | 114 | −8.277 | 39.083 | −48.861 | 1.00 | 23.89 | C |
| ATOM | 783 | OG | SER | | 114 | −7.906 | 40.392 | −48.473 | 1.00 | 30.53 | O |
| ATOM | 784 | C | SER | | 114 | −5.900 | 38.339 | −49.132 | 1.00 | 20.35 | C |
| ATOM | 785 | O | SER | | 114 | −5.756 | 38.049 | −50.320 | 1.00 | 19.83 | O |
| ATOM | 786 | N | SER | | 115 | −4.939 | 38.902 | −48.405 | 1.00 | 19.00 | N |
| ATOM | 787 | CA | SER | | 115 | −3.627 | 39.196 | −48.974 | 1.00 | 17.75 | C |
| ATOM | 788 | CB | SER | | 115 | −2.793 | 40.018 | −47.989 | 1.00 | 18.12 | C |
| ATOM | 789 | OG | SER | | 115 | −2.762 | 39.383 | −46.723 | 1.00 | 19.78 | O |
| ATOM | 790 | C | SER | | 115 | −2.897 | 37.897 | −49.310 | 1.00 | 16.56 | C |
| ATOM | 791 | O | SER | | 115 | −1.845 | 37.918 | −49.948 | 1.00 | 16.06 | O |
| ATOM | 792 | N | ALA | | 116 | −3.450 | 36.766 | −48.877 | 1.00 | 14.44 | N |
| ATOM | 793 | CA | ALA | | 116 | −2.841 | 35.475 | −49.174 | 1.00 | 13.90 | C |
| ATOM | 794 | CB | ALA | | 116 | −3.676 | 34.336 | −48.577 | 1.00 | 16.29 | C |
| ATOM | 795 | C | ALA | | 116 | −2.758 | 35.313 | −50.691 | 1.00 | 16.26 | C |
| ATOM | 796 | O | ALA | | 116 | −1.827 | 34.696 | −51.206 | 1.00 | 14.48 | O |
| ATOM | 797 | N | LYS | | 117 | −3.734 | 35.883 | −51.399 | 1.00 | 14.92 | N |
| ATOM | 798 | CA | LYS | | 117 | −3.785 | 35.792 | −52.856 | 1.00 | 15.91 | C |
| ATOM | 799 | CB | LYS | | 117 | −5.109 | 36.367 | −53.367 | 1.00 | 19.76 | C |
| ATOM | 800 | CG | LYS | | 117 | −6.325 | 35.632 | −52.833 | 1.00 | 23.04 | C |
| ATOM | 801 | CD | LYS | | 117 | −6.439 | 34.249 | −53.430 | 1.00 | 25.35 | C |
| ATOM | 802 | CE | LYS | | 117 | −7.560 | 33.448 | −52.768 | 1.00 | 29.29 | C |
| ATOM | 803 | NZ | LYS | | 117 | −7.151 | 32.900 | −51.437 | 1.00 | 25.83 | N |
| ATOM | 804 | C | LYS | | 117 | −2.611 | 36.489 | −53.537 | 1.00 | 16.41 | C |
| ATOM | 805 | O | LYS | | 117 | −2.340 | 36.246 | −54.714 | 1.00 | 14.80 | O |
| ATOM | 806 | N | ALA | | 118 | −1.927 | 37.355 | −52.794 | 1.00 | 15.41 | N |
| ATOM | 807 | CA | ALA | | 118 | −0.756 | 38.067 | −53.301 | 1.00 | 17.44 | C |
| ATOM | 808 | CB | ALA | | 118 | −0.950 | 39.575 | −53.154 | 1.00 | 17.29 | C |
| ATOM | 809 | C | ALA | | 118 | 0.494 | 37.624 | −52.530 | 1.00 | 16.94 | C |
| ATOM | 810 | O | ALA | | 118 | 1.398 | 38.423 | −52.282 | 1.00 | 16.10 | O |
| ATOM | 811 | N | ARG | | 119 | 0.536 | 36.351 | −52.151 | 1.00 | 16.46 | N |
| ATOM | 812 | CA | ARG | | 119 | 1.672 | 35.806 | −51.405 | 1.00 | 18.38 | C |
| ATOM | 813 | CB | ARG | | 119 | 2.941 | 35.835 | −52.274 | 1.00 | 20.28 | C |
| ATOM | 814 | CG | ARG | | 119 | 3.042 | 34.687 | −53.274 | 1.00 | 25.13 | C |
| ATOM | 815 | CD | ARG | | 119 | 3.989 | 34.993 | −54.440 | 1.00 | 28.20 | C |
| ATOM | 816 | NE | ARG | | 119 | 3.278 | 35.735 | −55.475 | 1.00 | 36.50 | N |
| ATOM | 817 | CZ | ARG | | 119 | 2.975 | 37.029 | −55.408 | 1.00 | 36.48 | C |
| ATOM | 818 | NH1 | ARG | | 119 | 3.339 | 37.755 | −54.358 | 1.00 | 39.04 | N |
| ATOM | 819 | NH2 | ARG | | 119 | 2.248 | 37.583 | −56.365 | 1.00 | 33.05 | N |
| ATOM | 820 | C | ARG | | 119 | 1.893 | 36.587 | −50.113 | 1.00 | 17.79 | C |
| ATOM | 821 | O | ARG | | 119 | 3.021 | 36.704 | −49.625 | 1.00 | 17.25 | O |
| ATOM | 822 | N | GLY | | 120 | 0.802 | 37.126 | −49.573 | 1.00 | 17.12 | N |
| ATOM | 823 | CA | GLY | | 120 | 0.858 | 37.877 | −48.331 | 1.00 | 18.51 | C |
| ATOM | 824 | C | GLY | | 120 | 1.296 | 39.326 | −48.417 | 1.00 | 19.02 | C |
| ATOM | 825 | O | GLY | | 120 | 1.308 | 40.024 | −47.401 | 1.00 | 18.59 | O |
| ATOM | 826 | N | ASP | | 121 | 1.639 | 39.792 | −49.615 | 1.00 | 17.66 | N |
| ATOM | 827 | CA | ASP | | 121 | 2.098 | 41.169 | −49.796 | 1.00 | 17.66 | C |
| ATOM | 828 | CB | ASP | | 121 | 2.584 | 41.376 | −51.235 | 1.00 | 18.36 | C |
| ATOM | 829 | CG | ASP | | 121 | 2.998 | 42.814 | −51.512 | 1.00 | 20.32 | C |
| ATOM | 830 | OD1 | ASP | | 121 | 3.740 | 43.390 | −50.691 | 1.00 | 18.59 | O |
| ATOM | 831 | OD2 | ASP | | 121 | 2.590 | 43.366 | −52.556 | 1.00 | 20.54 | O |
| ATOM | 832 | C | ASP | | 121 | 1.032 | 42.207 | −49.462 | 1.00 | 18.64 | C |
| ATOM | 833 | O | ASP | | 121 | −0.108 | 42.107 | −49.912 | 1.00 | 17.94 | O |
| ATOM | 834 | N | LEU | | 122 | 1.420 | 43.203 | −48.671 | 1.00 | 19.60 | N |
| ATOM | 835 | CA | LEU | | 122 | 0.517 | 44.273 | −48.262 | 1.00 | 21.00 | C |
| ATOM | 836 | CB | LEU | | 122 | 0.586 | 44.469 | −46.745 | 1.00 | 19.64 | C |
| ATOM | 837 | CG | LEU | | 122 | 0.071 | 43.312 | −45.890 | 1.00 | 18.59 | C |
| ATOM | 838 | CD1 | LEU | | 122 | 0.279 | 43.639 | −44.423 | 1.00 | 19.00 | C |
| ATOM | 839 | CD2 | LEU | | 122 | −1.394 | 43.067 | −46.181 | 1.00 | 20.13 | C |
| ATOM | 840 | C | LEU | | 122 | 0.849 | 45.594 | −48.940 | 1.00 | 22.71 | C |
| ATOM | 841 | O | LEU | | 122 | 0.142 | 46.585 | −48.760 | 1.00 | 24.31 | O |
| ATOM | 842 | N | GLY | | 123 | 1.926 | 45.611 | −49.717 | 1.00 | 24.34 | N |
| ATOM | 843 | CA | GLY | | 123 | 2.319 | 46.837 | −50.382 | 1.00 | 24.39 | C |
| ATOM | 844 | C | GLY | | 123 | 3.043 | 47.729 | −49.394 | 1.00 | 26.20 | C |
| ATOM | 845 | O | GLY | | 123 | 3.262 | 47.334 | −48.244 | 1.00 | 26.42 | O |
| ATOM | 846 | N | ALA | | 124 | 3.410 | 48.930 | −49.829 | 1.00 | 24.94 | N |
| ATOM | 847 | CA | ALA | | 124 | 4.127 | 49.865 | −48.970 | 1.00 | 26.18 | C |
| ATOM | 848 | CB | ALA | | 124 | 4.986 | 50.803 | −49.821 | 1.00 | 25.26 | C |
| ATOM | 849 | C | ALA | | 124 | 3.179 | 50.675 | −48.094 | 1.00 | 27.33 | C |
| ATOM | 850 | O | ALA | | 124 | 2.026 | 50.907 | −48.454 | 1.00 | 28.10 | O |
| ATOM | 851 | N | PHE | | 125 | 3.672 | 51.100 | −46.937 | 1.00 | 27.49 | N |
| ATOM | 852 | CA | PHE | | 125 | 2.867 | 51.892 | −46.021 | 1.00 | 28.28 | C |
| ATOM | 853 | CB | PHE | | 125 | 1.907 | 50.989 | −45.230 | 1.00 | 28.12 | C |
| ATOM | 854 | CG | PHE | | 125 | 2.592 | 49.926 | −44.401 | 1.00 | 27.68 | C |
| ATOM | 855 | CD1 | PHE | | 125 | 3.148 | 50.234 | −43.160 | 1.00 | 27.00 | C |
| ATOM | 856 | CD2 | PHE | | 125 | 2.665 | 48.612 | −44.859 | 1.00 | 27.80 | C |
| ATOM | 857 | CE1 | PHE | | 125 | 3.765 | 49.247 | −42.385 | 1.00 | 26.81 | C |
| ATOM | 858 | CE2 | PHE | | 125 | 3.280 | 47.617 | −44.093 | 1.00 | 27.95 | C |
| ATOM | 859 | CZ | PHE | | 125 | 3.831 | 47.936 | −42.853 | 1.00 | 26.30 | C |
| ATOM | 860 | C | PHE | | 125 | 3.749 | 52.695 | −45.081 | 1.00 | 28.75 | C |
| ATOM | 861 | O | PHE | | 125 | 4.948 | 52.431 | −44.950 | 1.00 | 29.57 | O |
| ATOM | 862 | N | SER | | 126 | 3.161 | 53.698 | −44.444 | 1.00 | 30.25 | N |
| ATOM | 863 | CA | SER | | 126 | 3.910 | 54.534 | −43.518 | 1.00 | 31.30 | C |
| ATOM | 864 | CB | SER | | 126 | 3.864 | 55.997 | −43.976 | 1.00 | 33.33 | C |
| ATOM | 865 | OG | SER | | 126 | 2.531 | 56.468 | −44.008 | 1.00 | 33.44 | O |
| ATOM | 866 | C | SER | | 126 | 3.284 | 54.394 | −42.138 | 1.00 | 30.33 | C |
| ATOM | 867 | O | SER | | 126 | 2.264 | 53.718 | −41.980 | 1.00 | 29.25 | O |
| ATOM | 868 | N | ARG | | 127 | 3.896 | 55.012 | −41.136 | 1.00 | 31.04 | N |
| ATOM | 869 | CA | ARG | | 127 | 3.343 | 54.935 | −39.793 | 1.00 | 32.68 | C |
| ATOM | 870 | CB | ARG | | 127 | 4.293 | 55.564 | −38.768 | 1.00 | 31.89 | C |
| ATOM | 871 | CG | ARG | | 127 | 5.498 | 54.699 | −38.407 | 1.00 | 30.14 | C |
| ATOM | 872 | CD | ARG | | 127 | 6.242 | 55.288 | −37.222 | 1.00 | 29.33 | C |
| ATOM | 873 | NE | ARG | | 127 | 7.395 | 54.495 | −36.801 | 1.00 | 29.19 | N |
| ATOM | 874 | CZ | ARG | | 127 | 7.329 | 53.386 | −36.069 | 1.00 | 27.71 | C |
| ATOM | 875 | NH1 | ARG | | 127 | 6.159 | 52.914 | −35.663 | 1.00 | 26.04 | N |
| ATOM | 876 | NH2 | ARG | | 127 | 8.446 | 52.757 | −35.727 | 1.00 | 27.67 | N |
| ATOM | 877 | C | ARG | | 127 | 2.005 | 55.660 | −39.780 | 1.00 | 34.62 | C |
| ATOM | 878 | O | ARG | | 127 | 1.810 | 56.634 | −40.508 | 1.00 | 35.97 | O |
| ATOM | 879 | N | GLY | | 128 | 1.081 | 55.168 | −38.964 | 1.00 | 35.85 | N |
| ATOM | 880 | CA | GLY | | 128 | −0.229 | 55.783 | −38.876 | 1.00 | 36.91 | C |
| ATOM | 881 | C | GLY | | 128 | −1.267 | 55.111 | −39.755 | 1.00 | 37.54 | C |
| ATOM | 882 | O | GLY | | 128 | −2.316 | 55.692 | −40.025 | 1.00 | 39.19 | O |
| ATOM | 883 | N | GLN | | 129 | −0.987 | 53.889 | −40.202 | 1.00 | 35.80 | N |
| ATOM | 884 | CA | GLN | | 129 | −1.923 | 53.169 | −41.059 | 1.00 | 35.32 | C |
| ATOM | 885 | CB | GLN | | 129 | −1.334 | 53.000 | −42.468 | 1.00 | 36.78 | C |
| ATOM | 886 | CG | GLN | | 129 | −0.790 | 54.287 | −43.094 | 1.00 | 38.82 | C |
| ATOM | 887 | CD | GLN | | 129 | −0.316 | 54.094 | −44.531 | 1.00 | 39.29 | C |
| ATOM | 888 | OE1 | GLN | | 129 | 0.330 | 54.966 | −45.108 | 1.00 | 41.53 | O |
| ATOM | 889 | NE2 | GLN | | 129 | −0.646 | 52.951 | −45.114 | 1.00 | 41.50 | N |
| ATOM | 890 | C | GLN | | 129 | −2.298 | 51.795 | −40.503 | 1.00 | 35.11 | C |
| ATOM | 891 | O | GLN | | 129 | −3.471 | 51.427 | −40.487 | 1.00 | 36.04 | O |
| ATOM | 892 | N | MET | | 130 | −1.301 | 51.043 | −40.041 | 1.00 | 33.14 | N |
| ATOM | 893 | CA | MET | | 130 | −1.531 | 49.700 | −39.509 | 1.00 | 31.66 | C |
| ATOM | 894 | CB | MET | | 130 | −0.364 | 48.786 | −39.898 | 1.00 | 31.81 | C |
| ATOM | 895 | CG | MET | | 130 | −0.037 | 48.775 | −41.379 | 1.00 | 32.58 | C |
| ATOM | 896 | SD | MET | | 130 | −1.345 | 48.040 | −42.365 | 1.00 | 37.52 | S |
| ATOM | 897 | CE | MET | | 130 | −0.543 | 47.922 | −43.967 | 1.00 | 34.65 | C |
| ATOM | 898 | C | MET | | 130 | −1.688 | 49.663 | −37.989 | 1.00 | 30.84 | C |
| ATOM | 899 | O | MET | | 130 | −1.312 | 50.607 | −37.296 | 1.00 | 31.94 | O |
| ATOM | 900 | N | GLN | | 131 | −2.243 | 48.567 | −37.478 | 1.00 | 29.69 | N |
| ATOM | 901 | CA | GLN | | 131 | −2.397 | 48.405 | −36.037 | 1.00 | 30.95 | C |
| ATOM | 902 | CB | GLN | | 131 | −2.945 | 47.015 | −35.708 | 1.00 | 32.79 | C |
| ATOM | 903 | CG | GLN | | 131 | −4.378 | 46.796 | −36.179 | 1.00 | 36.18 | C |
| ATOM | 904 | CD | GLN | | 131 | −4.953 | 45.467 | −35.722 | 1.00 | 39.27 | C |
| ATOM | 905 | OE1 | GLN | | 131 | −5.016 | 45.182 | −34.526 | 1.00 | 41.41 | O |
| ATOM | 906 | NE2 | GLN | | 131 | −5.376 | 44.648 | −36.675 | 1.00 | 39.96 | N |
| ATOM | 907 | C | GLN | | 131 | −0.997 | 48.588 | −35.458 | 1.00 | 30.83 | C |
| ATOM | 908 | O | GLN | | 131 | −0.018 | 48.084 | −36.012 | 1.00 | 28.51 | O |
| ATOM | 909 | N | LYS | | 132 | −0.907 | 49.311 | −34.348 | 1.00 | 29.13 | N |
| ATOM | 910 | CA | LYS | | 132 | 0.375 | 49.618 | −33.730 | 1.00 | 29.05 | C |
| ATOM | 911 | CB | LYS | | 132 | 0.152 | 50.300 | −32.379 | 1.00 | 29.79 | C |
| ATOM | 912 | CG | LYS | | 132 | 1.290 | 51.221 | −31.985 | 1.00 | 31.38 | C |
| ATOM | 913 | CD | LYS | | 132 | 1.452 | 52.323 | −33.017 | 1.00 | 33.54 | C |
| ATOM | 914 | CE | LYS | | 132 | 2.483 | 53.358 | −32.599 | 1.00 | 36.21 | C |
| ATOM | 915 | NZ | LYS | | 132 | 3.865 | 52.825 | −32.457 | 1.00 | 37.04 | N |
| ATOM | 916 | C | LYS | | 132 | 1.387 | 48.477 | −33.562 | 1.00 | 26.98 | C |
| ATOM | 917 | O | LYS | | 132 | 2.559 | 48.645 | −33.891 | 1.00 | 26.95 | O |
| ATOM | 918 | N | PRO | | 133 | 0.961 | 47.316 | −33.038 | 1.00 | 25.38 | N |
| ATOM | 919 | CD | PRO | | 133 | −0.350 | 46.934 | −32.488 | 1.00 | 26.31 | C |
| ATOM | 920 | CA | PRO | | 133 | 1.933 | 46.227 | −32.879 | 1.00 | 25.07 | C |
| ATOM | 921 | CB | PRO | | 133 | 1.096 | 45.101 | −32.280 | 1.00 | 25.70 | C |
| ATOM | 922 | CG | PRO | | 133 | 0.021 | 45.837 | −31.526 | 1.00 | 26.89 | C |
| ATOM | 923 | C | PRO | | 133 | 2.562 | 45.822 | −34.214 | 1.00 | 24.98 | C |
| ATOM | 924 | O | PRO | | 133 | 3.769 | 45.584 | −34.302 | 1.00 | 23.26 | O |
| ATOM | 925 | N | PHE | | 134 | 1.725 | 45.742 | −35.243 | 1.00 | 23.09 | N |
| ATOM | 926 | CA | PHE | | 134 | 2.166 | 45.367 | −36.581 | 1.00 | 21.04 | C |
| ATOM | 927 | CB | PHE | | 134 | 0.948 | 45.191 | −37.489 | 1.00 | 19.46 | C |
| ATOM | 928 | CG | PHE | | 134 | 1.261 | 44.548 | −38.811 | 1.00 | 18.37 | C |
| ATOM | 929 | CD1 | PHE | | 134 | 1.127 | 43.171 | −38.980 | 1.00 | 17.58 | C |
| ATOM | 930 | CD2 | PHE | | 134 | 1.704 | 45.313 | −39.883 | 1.00 | 17.64 | C |
| ATOM | 931 | CE1 | PHE | | 134 | 1.433 | 42.564 | −40.204 | 1.00 | 16.48 | C |
| ATOM | 932 | CE2 | PHE | | 134 | 2.012 | 44.719 | −41.106 | 1.00 | 17.26 | C |
| ATOM | 933 | CZ | PHE | | 134 | 1.877 | 43.343 | −41.267 | 1.00 | 16.26 | C |
| ATOM | 934 | C | PHE | | 134 | 3.072 | 46.464 | −37.130 | 1.00 | 20.47 | C |
| ATOM | 935 | O | PHE | | 134 | 4.143 | 46.193 | −37.679 | 1.00 | 19.21 | O |
| ATOM | 936 | N | GLU | | 135 | 2.643 | 47.713 | −36.972 | 1.00 | 19.62 | N |
| ATOM | 937 | CA | GLU | | 135 | 3.429 | 48.844 | −37.444 | 1.00 | 19.53 | C |
| ATOM | 938 | CB | GLU | | 135 | 2.720 | 50.162 | −37.127 | 1.00 | 20.05 | C |
| ATOM | 939 | CG | GLU | | 135 | 3.569 | 51.385 | −37.428 | 1.00 | 23.50 | C |
| ATOM | 940 | CD | GLU | | 135 | 2.907 | 52.678 | −36.985 | 1.00 | 27.69 | C |
| ATOM | 941 | OE1 | GLU | | 135 | 1.792 | 52.965 | −37.465 | 1.00 | 26.76 | O |
| ATOM | 942 | OE2 | GLU | | 135 | 3.506 | 53.401 | −36.158 | 1.00 | 28.46 | O |
| ATOM | 943 | C | GLU | | 135 | 4.819 | 48.868 | −36.821 | 1.00 | 19.33 | C |
| ATOM | 944 | O | GLU | | 135 | 5.826 | 48.954 | −37.532 | 1.00 | 17.25 | O |
| ATOM | 945 | N | ASP | | 136 | 4.879 | 48.807 | −35.493 | 1.00 | 18.94 | N |
| ATOM | 946 | CA | ASP | | 136 | 6.167 | 48.835 | −34.802 | 1.00 | 17.95 | C |
| ATOM | 947 | CB | ASP | | 136 | 5.973 | 48.771 | −33.282 | 1.00 | 18.95 | C |
| ATOM | 948 | CG | ASP | | 136 | 5.416 | 50.068 | −32.705 | 1.00 | 22.69 | C |
| ATOM | 949 | OD1 | ASP | | 136 | 5.499 | 51.110 | −33.388 | 1.00 | 21.20 | O |
| ATOM | 950 | OD2 | ASP | | 136 | 4.908 | 50.049 | −31.566 | 1.00 | 22.27 | O |
| ATOM | 951 | C | ASP | | 136 | 7.097 | 47.711 | −35.249 | 1.00 | 15.96 | C |
| ATOM | 952 | O | ASP | | 136 | 8.284 | 47.939 | −35.482 | 1.00 | 15.72 | O |
| ATOM | 953 | N | ALA | | 137 | 6.564 | 46.501 | −35.367 | 1.00 | 16.06 | N |
| ATOM | 954 | CA | ALA | | 137 | 7.379 | 45.367 | −35.795 | 1.00 | 15.87 | C |
| ATOM | 955 | CB | ALA | | 137 | 6.558 | 44.078 | −35.743 | 1.00 | 15.26 | C |
| ATOM | 956 | C | ALA | | 137 | 7.891 | 45.614 | −37.216 | 1.00 | 15.35 | C |
| ATOM | 957 | O | ALA | | 137 | 9.070 | 45.424 | −37.503 | 1.00 | 16.28 | O |
| ATOM | 958 | N | SER | | 138 | 7.001 | 46.060 | −38.096 | 1.00 | 15.85 | N |
| ATOM | 959 | CA | SER | | 138 | 7.366 | 46.325 | −39.482 | 1.00 | 17.04 | C |
| ATOM | 960 | CB | SER | | 138 | 6.158 | 46.832 | −40.269 | 1.00 | 16.28 | C |
| ATOM | 961 | OG | SER | | 138 | 5.146 | 45.847 | −40.324 | 1.00 | 14.90 | O |
| ATOM | 962 | C | SER | | 138 | 8.504 | 47.323 | −39.628 | 1.00 | 19.07 | C |
| ATOM | 963 | O | SER | | 138 | 9.438 | 47.084 | −40.391 | 1.00 | 18.39 | O |
| ATOM | 964 | N | PHE | | 139 | 8.429 | 48.442 | −38.908 | 1.00 | 18.59 | N |
| ATOM | 965 | CA | PHE | | 139 | 9.472 | 49.457 | −38.999 | 1.00 | 20.09 | C |
| ATOM | 966 | CB | PHE | | 139 | 8.938 | 50.824 | −38.552 | 1.00 | 20.73 | C |
| ATOM | 967 | CG | PHE | | 139 | 8.174 | 51.544 | −39.626 | 1.00 | 21.76 | C |
| ATOM | 968 | CD1 | PHE | | 139 | 6.857 | 51.208 | −39.908 | 1.00 | 22.50 | C |
| ATOM | 969 | CD2 | PHE | | 139 | 8.800 | 52.514 | −40.408 | 1.00 | 22.93 | C |
| ATOM | 970 | CE1 | PHE | | 139 | 6.169 | 51.822 | −40.958 | 1.00 | 22.33 | C |
| ATOM | 971 | CE2 | PHE | | 139 | 8.124 | 53.132 | −41.458 | 1.00 | 22.59 | C |
| ATOM | 972 | CZ | PHE | | 139 | 6.806 | 52.784 | −41.733 | 1.00 | 20.81 | C |
| ATOM | 973 | C | PHE | | 139 | 10.748 | 49.122 | −38.241 | 1.00 | 20.34 | C |
| ATOM | 974 | O | PHE | | 139 | 11.782 | 49.748 | −38.467 | 1.00 | 22.50 | O |
| ATOM | 975 | N | ALA | | 140 | 10.681 | 48.132 | −37.355 | 1.00 | 19.71 | N |
| ATOM | 976 | CA | ALA | | 140 | 11.853 | 47.719 | −36.587 | 1.00 | 19.37 | C |
| ATOM | 977 | CB | ALA | | 140 | 11.425 | 47.180 | −35.231 | 1.00 | 18.59 | C |
| ATOM | 978 | C | ALA | | 140 | 12.648 | 46.659 | −37.351 | 1.00 | 19.95 | C |
| ATOM | 979 | O | ALA | | 140 | 13.842 | 46.469 | −37.107 | 1.00 | 19.76 | O |
| ATOM | 980 | N | LEU | | 141 | 11.982 | 45.972 | −38.274 | 1.00 | 19.78 | N |
| ATOM | 981 | CA | LEU | | 141 | 12.637 | 44.953 | −39.088 | 1.00 | 20.98 | C |
| ATOM | 982 | CB | LEU | | 141 | 11.606 | 44.130 | −39.871 | 1.00 | 19.49 | C |
| ATOM | 983 | CG | LEU | | 141 | 10.740 | 43.130 | −39.106 | 1.00 | 19.91 | C |
| ATOM | 984 | CD1 | LEU | | 141 | 9.622 | 42.623 | −40.011 | 1.00 | 17.89 | C |
| ATOM | 985 | CD2 | LEU | | 141 | 11.609 | 41.975 | −38.617 | 1.00 | 18.95 | C |
| ATOM | 986 | C | LEU | | 141 | 13.576 | 45.602 | −40.089 | 1.00 | 22.95 | C |
| ATOM | 987 | O | LEU | | 141 | 13.316 | 46.706 | −40.567 | 1.00 | 22.66 | O |
| ATOM | 988 | N | ARG | | 142 | 14.673 | 44.918 | −40.396 | 1.00 | 24.04 | N |
| ATOM | 989 | CA | ARG | | 142 | 15.622 | 45.409 | −41.382 | 1.00 | 25.12 | C |
| ATOM | 990 | CB | ARG | | 142 | 17.020 | 44.849 | −41.089 | 1.00 | 28.10 | C |
| ATOM | 991 | CG | ARG | | 142 | 17.569 | 45.213 | −39.700 | 1.00 | 29.80 | C |
| ATOM | 992 | CD | ARG | | 142 | 18.970 | 44.637 | −39.478 | 1.00 | 33.71 | C |
| ATOM | 993 | NE | ARG | | 142 | 18.963 | 43.177 | −39.413 | 1.00 | 35.33 | N |
| ATOM | 994 | CZ | ARG | | 142 | 18.821 | 42.476 | −38.292 | 1.00 | 36.74 | C |
| ATOM | 995 | NH1 | ARG | | 142 | 18.682 | 43.101 | −37.132 | 1.00 | 35.28 | N |
| ATOM | 996 | NH2 | ARG | | 142 | 18.804 | 41.149 | −38.334 | 1.00 | 35.75 | N |
| ATOM | 997 | C | ARG | | 142 | 15.088 | 44.863 | −42.710 | 1.00 | 25.76 | C |
| ATOM | 998 | O | ARG | | 142 | 14.313 | 43.906 | −42.714 | 1.00 | 24.03 | O |
| ATOM | 999 | N | THR | | 143 | 15.476 | 45.462 | −43.831 | 1.00 | 24.42 | N |
| ATOM | 1000 | CA | THR | | 143 | 15.002 | 44.985 | −45.125 | 1.00 | 24.26 | C |
| ATOM | 1001 | CB | THR | | 143 | 15.547 | 45.842 | −46.277 | 1.00 | 26.49 | C |
| ATOM | 1002 | OG1 | THR | | 143 | 15.077 | 47.185 | −46.129 | 1.00 | 30.99 | O |
| ATOM | 1003 | CG2 | THR | | 143 | 15.078 | 45.299 | −47.615 | 1.00 | 27.13 | C |
| ATOM | 1004 | C | THR | | 143 | 15.437 | 43.540 | −45.335 | 1.00 | 23.33 | C |
| ATOM | 1005 | O | THR | | 143 | 16.618 | 43.221 | −45.226 | 1.00 | 21.96 | O |
| ATOM | 1006 | N | GLY | | 144 | 14.469 | 42.672 | −45.619 | 1.00 | 21.58 | N |
| ATOM | 1007 | CA | GLY | | 144 | 14.762 | 41.266 | −45.828 | 1.00 | 19.98 | C |
| ATOM | 1008 | C | GLY | | 144 | 14.558 | 40.414 | −44.589 | 1.00 | 20.03 | C |
| ATOM | 1009 | O | GLY | | 144 | 14.491 | 39.184 | −44.678 | 1.00 | 20.32 | O |
| ATOM | 1010 | N | GLU | | 145 | 14.449 | 41.064 | −43.432 | 1.00 | 18.28 | N |
| ATOM | 1011 | CA | GLU | | 145 | 14.275 | 40.363 | −42.164 | 1.00 | 18.24 | C |
| ATOM | 1012 | CB | GLU | | 145 | 14.798 | 41.236 | −41.013 | 1.00 | 18.38 | C |
| ATOM | 1013 | CG | GLU | | 145 | 14.567 | 40.646 | −39.632 | 1.00 | 23.98 | C |
| ATOM | 1014 | CD | GLU | | 145 | 15.205 | 41.458 | −38.520 | 1.00 | 26.76 | C |
| ATOM | 1015 | OE1 | GLU | | 145 | 15.393 | 42.683 | −38.704 | 1.00 | 25.29 | O |
| ATOM | 1016 | OE2 | GLU | | 145 | 15.505 | 40.869 | −37.454 | 1.00 | 26.16 | O |
| ATOM | 1017 | C | GLU | | 145 | 12.832 | 39.936 | −41.879 | 1.00 | 17.64 | C |
| ATOM | 1018 | O | GLU | | 145 | 11.876 | 40.569 | −42.337 | 1.00 | 18.16 | O |
| ATOM | 1019 | N | MET | | 146 | 12.691 | 38.853 | −41.122 | 1.00 | 15.35 | N |
| ATOM | 1020 | CA | MET | | 146 | 11.386 | 38.317 | −40.762 | 1.00 | 15.06 | C |
| ATOM | 1021 | CB | MET | | 146 | 11.276 | 36.855 | −41.221 | 1.00 | 15.40 | C |
| ATOM | 1022 | CG | MET | | 146 | 9.901 | 36.224 | −41.019 | 1.00 | 14.97 | C |
| ATOM | 1023 | SD | MET | | 146 | 9.864 | 34.522 | −41.623 | 1.00 | 16.26 | S |
| ATOM | 1024 | CE | MET | | 146 | 10.613 | 33.673 | −40.228 | 1.00 | 15.69 | C |
| ATOM | 1025 | C | MET | | 146 | 11.170 | 38.408 | −39.252 | 1.00 | 14.21 | C |
| ATOM | 1026 | O | MET | | 146 | 12.103 | 38.248 | −38.456 | 1.00 | 12.74 | O |
| ATOM | 1027 | N | SER | | 147 | 9.929 | 38.663 | −38.861 | 1.00 | 13.22 | N |
| ATOM | 1028 | CA | SER | | 147 | 9.595 | 38.789 | −37.451 | 1.00 | 14.04 | C |
| ATOM | 1029 | CB | SER | | 147 | 8.328 | 39.622 | −37.285 | 1.00 | 12.58 | C |
| ATOM | 1030 | OG | SER | | 147 | 7.197 | 38.817 | −37.614 | 1.00 | 13.57 | O |
| ATOM | 1031 | C | SER | | 147 | 9.308 | 37.438 | −36.836 | 1.00 | 13.44 | C |
| ATOM | 1032 | O | SER | | 147 | 9.272 | 36.422 | −37.523 | 1.00 | 13.67 | O |
| ATOM | 1033 | N | GLY | | 148 | 9.102 | 37.455 | −35.524 | 1.00 | 15.04 | N |
| ATOM | 1034 | CA | GLY | | 148 | 8.711 | 36.255 | −34.812 | 1.00 | 15.40 | C |
| ATOM | 1035 | C | GLY | | 148 | 7.204 | 36.441 | −34.722 | 1.00 | 16.85 | C |
| ATOM | 1036 | O | GLY | | 148 | 6.658 | 37.265 | −35.461 | 1.00 | 16.08 | O |
| ATOM | 1037 | N | PRO | | 149 | 6.495 | 35.714 | −33.849 | 1.00 | 18.97 | N |
| ATOM | 1038 | CD | PRO | | 149 | 6.939 | 34.580 | −33.021 | 1.00 | 18.56 | C |
| ATOM | 1039 | CA | PRO | | 149 | 5.038 | 35.899 | −33.761 | 1.00 | 18.39 | C |
| ATOM | 1040 | CB | PRO | | 149 | 4.605 | 34.809 | −32.777 | 1.00 | 19.39 | C |
| ATOM | 1041 | CG | PRO | | 149 | 5.681 | 33.748 | −32.929 | 1.00 | 19.27 | C |
| ATOM | 1042 | C | PRO | | 149 | 4.682 | 37.302 | −33.254 | 1.00 | 19.10 | C |
| ATOM | 1043 | O | PRO | | 149 | 5.177 | 37.729 | −32.211 | 1.00 | 20.03 | O |
| ATOM | 1044 | N | VAL | | 150 | 3.835 | 38.014 | −33.996 | 1.00 | 16.13 | N |
| ATOM | 1045 | CA | VAL | | 150 | 3.408 | 39.362 | −33.609 | 1.00 | 15.88 | C |
| ATOM | 1046 | CB | VAL | | 150 | 3.749 | 40.401 | −34.700 | 1.00 | 14.93 | C |
| ATOM | 1047 | CG1 | VAL | | 150 | 3.159 | 41.762 | −34.344 | 1.00 | 16.97 | C |
| ATOM | 1048 | CG2 | VAL | | 150 | 5.261 | 40.519 | −34.838 | 1.00 | 16.68 | C |
| ATOM | 1049 | C | VAL | | 150 | 1.897 | 39.328 | −33.391 | 1.00 | 17.21 | C |
| ATOM | 1050 | O | VAL | | 150 | 1.151 | 38.932 | −34.279 | 1.00 | 16.07 | O |
| ATOM | 1051 | N | PHE | | 151 | 1.459 | 39.738 | −32.204 | 1.00 | 16.55 | N |
| ATOM | 1052 | CA | PHE | | 151 | 0.042 | 39.715 | −31.861 | 1.00 | 18.40 | C |
| ATOM | 1053 | CB | PHE | | 151 | −0.140 | 39.322 | −30.385 | 1.00 | 18.03 | C |
| ATOM | 1054 | CG | PHE | | 151 | 0.501 | 38.016 | −30.013 | 1.00 | 18.94 | C |
| ATOM | 1055 | CD1 | PHE | | 151 | 1.881 | 37.919 | −29.883 | 1.00 | 18.93 | C |
| ATOM | 1056 | CD2 | PHE | | 151 | −0.275 | 36.883 | −29.792 | 1.00 | 20.23 | C |
| ATOM | 1057 | CE1 | PHE | | 151 | 2.482 | 36.711 | −29.537 | 1.00 | 20.92 | C |
| ATOM | 1058 | CE2 | PHE | | 151 | 0.317 | 35.667 | −29.445 | 1.00 | 21.68 | C |
| ATOM | 1059 | CZ | PHE | | 151 | 1.699 | 35.584 | −29.318 | 1.00 | 22.21 | C |
| ATOM | 1060 | C | PHE | | 151 | −0.679 | 41.039 | −32.095 | 1.00 | 18.85 | C |
| ATOM | 1061 | O | PHE | | 151 | −0.167 | 42.104 | −31.752 | 1.00 | 20.63 | O |
| ATOM | 1062 | N | THR | | 152 | −1.863 | 40.962 | −32.694 | 1.00 | 20.44 | N |
| ATOM | 1063 | CA | THR | | 152 | −2.699 | 42.142 | −32.929 | 1.00 | 20.95 | C |
| ATOM | 1064 | CB | THR | | 152 | 2.633 | 42.666 | −34.378 | 1.00 | 19.75 | C |
| ATOM | 1065 | OG1 | THR | | 152 | −3.235 | 41.704 | −35.252 | 1.00 | 19.21 | O |
| ATOM | 1066 | CG2 | THR | | 152 | −1.202 | 42.943 | −34.798 | 1.00 | 21.65 | C |
| ATOM | 1067 | C | THR | | 152 | −4.129 | 41.680 | −32.695 | 1.00 | 20.68 | C |
| ATOM | 1068 | O | THR | | 152 | −4.376 | 40.491 | −32.496 | 1.00 | 18.54 | O |
| ATOM | 1069 | N | ASP | | 153 | −5.078 | 42.608 | −32.735 | 1.00 | 23.80 | N |
| ATOM | 1070 | CA | ASP | | 153 | −6.468 | 42.234 | −32.534 | 1.00 | 26.27 | C |
| ATOM | 1071 | CB | ASP | | 153 | −7.341 | 43.484 | −32.390 | 1.00 | 29.45 | C |
| ATOM | 1072 | CG | ASP | | 153 | −7.015 | 44.275 | −31.137 | 1.00 | 34.74 | C |
| ATOM | 1073 | OD1 | ASP | | 153 | −6.820 | 43.639 | −30.080 | 1.00 | 35.79 | O |
| ATOM | 1074 | OD2 | ASP | | 153 | −6.960 | 45.524 | −31.200 | 1.00 | 37.90 | O |
| ATOM | 1075 | C | ASP | | 153 | −6.985 | 41.353 | −33.668 | 1.00 | 26.67 | C |
| ATOM | 1076 | O | ASP | | 153 | −8.004 | 40.678 | −33.521 | 1.00 | 28.28 | O |
| ATOM | 1077 | N | SER | | 154 | −6.279 | 41.348 | −34.798 | 1.00 | 23.94 | N |
| ATOM | 1078 | CA | SER | | 154 | −6.690 | 40.530 | −35.934 | 1.00 | 22.63 | C |
| ATOM | 1079 | CB | SER | | 154 | −6.085 | 41.068 | −37.234 | 1.00 | 24.28 | C |
| ATOM | 1080 | OG | SER | | 154 | −6.515 | 42.393 | −37.482 | 1.00 | 28.47 | O |
| ATOM | 1081 | C | SER | | 154 | −6.268 | 39.077 | −35.753 | 1.00 | 22.11 | C |
| ATOM | 1082 | O | SER | | 154 | −6.944 | 38.161 | −36.230 | 1.00 | 21.34 | O |
| ATOM | 1083 | N | GLY | | 155 | −5.148 | 38.874 | −35.063 | 1.00 | 19.10 | N |
| ATOM | 1084 | CA | GLY | | 155 | −4.649 | 37.528 | −34.840 | 1.00 | 19.13 | C |
| ATOM | 1085 | C | GLY | | 155 | −3.145 | 37.535 | −34.633 | 1.00 | 17.68 | C |
| ATOM | 1086 | O | GLY | | 155 | −2.593 | 38.516 | −34.140 | 1.00 | 18.75 | O |
| ATOM | 1087 | N | ILE | | 156 | −2.479 | 36.447 | −35.007 | 1.00 | 17.75 | N |
| ATOM | 1088 | CA | ILE | | 156 | −1.028 | 36.355 | −34.851 | 1.00 | 16.35 | C |
| ATOM | 1089 | CB | ILE | | 156 | −0.627 | 35.044 | −34.136 | 1.00 | 17.18 | C |
| ATOM | 1090 | CG2 | ILE | | 156 | 0.867 | 35.067 | −33.793 | 1.00 | 17.57 | C |
| ATOM | 1091 | CG1 | ILE | | 156 | −1.442 | 34.886 | −32.844 | 1.00 | 18.69 | C |
| ATOM | 1092 | CD1 | ILE | | 156 | −1.231 | 33.554 | −32.149 | 1.00 | 18.75 | C |
| ATOM | 1093 | C | ILE | | 156 | −0.391 | 36.392 | −36.241 | 1.00 | 16.94 | C |
| ATOM | 1094 | O | ILE | | 156 | −0.806 | 35.652 | −37.141 | 1.00 | 16.38 | O |
| ATOM | 1095 | N | HIS | | 157 | 0.620 | 37.244 | −36.405 | 1.00 | 16.22 | N |
| ATOM | 1096 | CA | HIS | | 157 | 1.289 | 37.406 | −37.697 | 1.00 | 15.33 | C |
| ATOM | 1097 | CB | HIS | | 157 | 1.263 | 38.870 | −38.149 | 1.00 | 16.86 | C |
| ATOM | 1098 | CG | HIS | | 157 | −0.060 | 39.554 | −38.004 | 1.00 | 19.00 | C |
| ATOM | 1099 | CD2 | HIS | | 157 | −0.750 | 39.943 | −36.906 | 1.00 | 19.48 | C |
| ATOM | 1100 | ND1 | HIS | | 157 | −0.773 | 40.014 | −39.090 | 1.00 | 18.92 | N |
| ATOM | 1101 | CE1 | HIS | | 157 | −1.844 | 40.662 | −38.666 | 1.00 | 20.43 | C |
| ATOM | 1102 | NE2 | HIS | | 157 | −1.854 | 40.634 | −37.345 | 1.00 | 18.30 | N |
| ATOM | 1103 | C | HIS | | 157 | 2.772 | 37.034 | −37.741 | 1.00 | 15.90 | C |
| ATOM | 1104 | O | HIS | | 157 | 3.474 | 37.078 | −36.733 | 1.00 | 16.36 | O |
| ATOM | 1105 | N | ILE | | 158 | 3.226 | 36.697 | −38.944 | 1.00 | 14.48 | N |
| ATOM | 1106 | CA | ILE | | 158 | 4.635 | 36.466 | −39.217 | 1.00 | 14.98 | C |
| ATOM | 1107 | CB | ILE | | 158 | 4.934 | 35.115 | −39.894 | 1.00 | 15.09 | C |
| ATOM | 1108 | CG2 | ILE | | 158 | 6.422 | 35.048 | −40.245 | 1.00 | 16.74 | C |
| ATOM | 1109 | CG1 | ILE | | 158 | 4.586 | 33.959 | −38.959 | 1.00 | 17.21 | C |
| ATOM | 1110 | CD1 | ILE | | 158 | 4.711 | 32.589 | −39.623 | 1.00 | 15.82 | C |
| ATOM | 1111 | C | ILE | | 158 | 4.807 | 37.575 | −40.249 | 1.00 | 12.93 | C |
| ATOM | 1112 | O | ILE | | 158 | 4.091 | 37.608 | −41.250 | 1.00 | 14.94 | O |
| ATOM | 1113 | N | ILE | | 159 | 5.729 | 38.495 | −40.003 | 1.00 | 13.20 | N |
| ATOM | 1114 | CA | ILE | | 159 | 5.925 | 39.612 | −40.915 | 1.00 | 13.52 | C |
| ATOM | 1115 | CB | ILE | | 159 | 5.823 | 40.959 | −40.152 | 1.00 | 13.34 | C |
| ATOM | 1116 | CG2 | ILE | | 159 | 5.902 | 42.130 | −41.131 | 1.00 | 13.88 | C |
| ATOM | 1117 | CG1 | ILE | | 159 | 4.507 | 41.008 | −39.370 | 1.00 | 16.65 | C |
| ATOM | 1118 | CD1 | ILE | | 159 | 4.389 | 42.198 | −38.423 | 1.00 | 14.49 | C |
| ATOM | 1119 | C | ILE | | 159 | 7.276 | 39.545 | −41.618 | 1.00 | 13.96 | C |
| ATOM | 1120 | O | ILE | | 159 | 8.291 | 39.213 | −41.007 | 1.00 | 14.96 | O |
| ATOM | 1121 | N | LEU | | 160 | 7.276 | 39.850 | −42.910 | 1.00 | 13.19 | N |
| ATOM | 1122 | CA | LEU | | 160 | 8.510 | 39.848 | −43.683 | 1.00 | 14.93 | C |
| ATOM | 1123 | CB | LEU | | 160 | 8.455 | 38.787 | −44.790 | 1.00 | 14.48 | C |
| ATOM | 1124 | CG | LEU | | 160 | 9.654 | 38.749 | −45.745 | 1.00 | 15.71 | C |
| ATOM | 1125 | CD1 | LEU | | 160 | 10.889 | 38.259 | −44.997 | 1.00 | 16.43 | C |
| ATOM | 1126 | CD2 | LEU | | 160 | 9.356 | 37.833 | −46.924 | 1.00 | 16.10 | C |
| ATOM | 1127 | C | LEU | | 160 | 8.657 | 41.222 | −44.311 | 1.00 | 15.40 | C |
| ATOM | 1128 | O | LEU | | 160 | 7.807 | 41.636 | −45.090 | 1.00 | 15.07 | O |
| ATOM | 1129 | N | ARG | | 161 | 9.708 | 41.950 | −43.955 | 1.00 | 17.20 | N |
| ATOM | 1130 | CA | ARG | | 161 | 9.902 | 43.260 | −44.560 | 1.00 | 18.43 | C |
| ATOM | 1131 | CB | ARG | | 161 | 10.712 | 44.192 | −43.660 | 1.00 | 18.62 | C |
| ATOM | 1132 | CG | ARG | | 161 | 10.886 | 45.551 | −44.311 | 1.00 | 21.13 | C |
| ATOM | 1133 | CD | ARG | | 161 | 11.719 | 46.524 | −43.501 | 1.00 | 23.54 | C |
| ATOM | 1134 | NE | ARG | | 161 | 12.050 | 47.687 | −44.322 | 1.00 | 24.30 | N |
| ATOM | 1135 | CZ | ARG | | 161 | 12.967 | 48.595 | −44.006 | 1.00 | 26.72 | C |
| ATOM | 1136 | NH1 | ARG | | 161 | 13.653 | 48.487 | −42.874 | 1.00 | 26.00 | N |
| ATOM | 1137 | NH2 | ARG | | 161 | 13.217 | 49.600 | −44.838 | 1.00 | 26.46 | N |
| ATOM | 1138 | C | ARG | | 161 | 10.633 | 43.051 | −45.879 | 1.00 | 18.60 | C |
| ATOM | 1139 | O | ARG | | 161 | 11.733 | 42.493 | −45.908 | 1.00 | 19.14 | O |
| ATOM | 1140 | N | THR | | 162 | 10.016 | 43.492 | −46.968 | 1.00 | 18.87 | N |
| ATOM | 1141 | CA | THR | | 162 | 10.596 | 43.319 | −48.293 | 1.00 | 20.27 | C |
| ATOM | 1142 | CB | THR | | 162 | 9.534 | 42.823 | −49.282 | 1.00 | 19.85 | C |
| ATOM | 1143 | OG1 | THR | | 162 | 8.442 | 43.750 | −49.312 | 1.00 | 20.20 | O |
| ATOM | 1144 | CG2 | THR | | 162 | 9.018 | 41.447 | −48.861 | 1.00 | 22.03 | C |
| ATOM | 1145 | C | THR | | 162 | 11.251 | 44.576 | −48.867 | 1.00 | 21.08 | C |
| ATOM | 1146 | O | THR | | 162 | 12.066 | 44.483 | −49.781 | 1.00 | 21.33 | O |
| ATOM | 1147 | N | GLU | | 163 | 10.881 | 45.742 | −48.341 | 1.00 | 22.73 | N |
| ATOM | 1148 | CA | GLU | | 163 | 11.443 | 47.013 | −48.798 | 1.00 | 24.59 | C |
| ATOM | 1149 | CB | GLU | | 163 | 10.574 | 47.617 | −49.908 | 1.00 | 26.38 | C |
| ATOM | 1150 | CG | GLU | | 163 | 10.664 | 46.915 | −51.252 | 1.00 | 27.57 | C |
| ATOM | 1151 | CD | GLU | | 163 | 9.691 | 47.485 | −52.277 | 1.00 | 29.28 | C |
| ATOM | 1152 | OE1 | GLU | | 163 | 9.265 | 48.651 | −52.120 | 1.00 | 31.89 | O |
| ATOM | 1153 | OE2 | GLU | | 163 | 9.362 | 46.771 | −53.247 | 1.00 | 30.85 | O |
| ATOM | 1154 | C | GLU | | 163 | 11.521 | 47.998 | −47.639 | 1.00 | 25.70 | C |
| ATOM | 1155 | O | GLU | | 163 | 10.748 | 47.822 | −46.679 | 1.00 | 25.93 | O |
| ATOM | 1156 | OXT | GLU | | 163 | 12.333 | 48.946 | −47.711 | 1.00 | 26.60 | O |
| ATOM | 1157 | OH2 | TIP | S | 1 | −3.169 | 46.912 | −39.226 | 1.00 | 39.98 | O |
| ATOM | 1158 | OH2 | TIP | S | 2 | −9.187 | 43.900 | −35.953 | 1.00 | 50.37 | O |
| ATOM | 1159 | OH2 | TIP | S | 3 | −9.804 | 41.479 | −39.377 | 1.00 | 35.60 | O |
| ATOM | 1160 | OH2 | TIP | S | 4 | −9.694 | 39.156 | −37.013 | 1.00 | 33.00 | O |
| ATOM | 1161 | OH2 | TIP | S | 5 | −10.351 | 40.786 | −42.439 | 1.00 | 37.69 | O |
| ATOM | 1162 | OH2 | TIP | S | 6 | −9.828 | 37.555 | −43.281 | 1.00 | 40.25 | O |
| ATOM | 1163 | OH2 | TIP | S | 7 | −9.134 | 39.541 | −45.417 | 1.00 | 39.63 | O |
| ATOM | 1164 | OH2 | TIP | S | 8 | −9.883 | 36.059 | −45.787 | 1.00 | 25.86 | O |
| ATOM | 1165 | OH2 | TIP | S | 9 | −11.183 | 37.138 | −47.825 | 1.00 | 51.31 | O |
| ATOM | 1166 | OH2 | TIP | S | 10 | −13.134 | 37.200 | −44.801 | 1.00 | 57.81 | O |
| ATOM | 1167 | OH2 | TIP | S | 11 | −13.681 | 36.911 | −48.670 | 1.00 | 54.32 | O |
| ATOM | 1168 | OH2 | TIP | S | 12 | −2.662 | 45.936 | −48.636 | 1.00 | 46.71 | O |
| ATOM | 1169 | OH2 | TIP | S | 13 | −2.729 | 42.968 | −50.167 | 1.00 | 46.64 | O |
| ATOM | 1170 | OH2 | TIP | S | 14 | −6.847 | 39.305 | −52.558 | 1.00 | 37.75 | O |
| ATOM | 1171 | OH2 | TIP | S | 15 | −2.348 | 50.877 | −45.468 | 1.00 | 50.94 | O |
| ATOM | 1172 | OH2 | TIP | S | 16 | −0.027 | 49.396 | −49.152 | 1.00 | 40.82 | O |
| ATOM | 1173 | OH2 | TIP | S | 17 | −3.752 | 45.286 | −32.311 | 1.00 | 28.84 | O |
| ATOM | 1174 | OH2 | TIP | S | 18 | −5.994 | 47.670 | −33.002 | 1.00 | 43.94 | O |
| ATOM | 1175 | OH2 | TIP | S | 19 | 7.767 | 41.156 | −27.330 | 1.00 | 16.45 | O |
| ATOM | 1176 | OH2 | TIP | S | 20 | −4.354 | 32.054 | −51.696 | 1.00 | 20.07 | O |
| ATOM | 1177 | OH2 | TIP | S | 21 | 5.998 | 42.383 | −49.731 | 1.00 | 19.29 | O |
| ATOM | 1178 | OH2 | TIP | S | 22 | 17.254 | 30.420 | −41.338 | 1.00 | 16.42 | O |
| ATOM | 1179 | OH2 | TIP | S | 23 | −1.421 | 37.146 | −45.955 | 1.00 | 19.88 | O |
| ATOM | 1180 | OH2 | TIP | S | 24 | 15.467 | 44.230 | −36.632 | 1.00 | 20.60 | O |
| ATOM | 1181 | OH2 | TIP | S | 25 | 13.875 | 42.090 | −35.571 | 1.00 | 18.18 | O |
| ATOM | 1182 | OH2 | TIP | S | 26 | −0.712 | 36.488 | −56.932 | 1.00 | 16.98 | O |
| ATOM | 1183 | OH2 | TIP | S | 27 | 5.199 | 38.069 | −50.303 | 1.00 | 19.42 | O |
| ATOM | 1184 | OH2 | TIP | S | 28 | 1.075 | 34.438 | −56.473 | 1.00 | 24.74 | O |
| ATOM | 1185 | OH2 | TIP | S | 29 | 12.785 | 30.169 | −46.437 | 1.00 | 20.29 | O |
| ATOM | 1186 | OH2 | TIP | S | 30 | −3.063 | 27.789 | −40.135 | 1.00 | 23.20 | O |
| ATOM | 1187 | OH2 | TIP | S | 31 | 9.513 | 28.972 | −34.350 | 1.00 | 20.46 | O |
| ATOM | 1188 | OH2 | TIP | S | 32 | 1.531 | 52.084 | −39.925 | 1.00 | 32.18 | O |
| ATOM | 1189 | OH2 | TIP | S | 33 | 2.459 | 24.898 | −38.457 | 1.00 | 21.37 | O |
| ATOM | 1190 | OH2 | TIP | S | 34 | −0.315 | 33.894 | −53.978 | 1.00 | 23.30 | O |
| ATOM | 1191 | OH2 | TIP | S | 35 | 17.426 | 32.582 | −37.015 | 1.00 | 29.07 | O |
| ATOM | 1192 | OH2 | TIP | S | 36 | −8.210 | 28.942 | −40.858 | 1.00 | 23.67 | O |
| ATOM | 1193 | OH2 | TIP | S | 37 | 6.182 | 35.858 | −29.883 | 1.00 | 28.85 | O |
| ATOM | 1194 | OH2 | TIP | S | 38 | 5.369 | 45.061 | −32.140 | 1.00 | 24.36 | O |
| ATOM | 1195 | OH2 | TIP | S | 39 | 15.086 | 31.328 | −31.812 | 1.00 | 24.42 | O |
| ATOM | 1196 | OH2 | TIP | S | 40 | −3.751 | 37.660 | −31.387 | 1.00 | 25.58 | O |
| ATOM | 1197 | OH2 | TIP | S | 41 | −2.468 | 23.978 | −49.241 | 1.00 | 29.30 | O |
| ATOM | 1198 | OH2 | TIP | S | 42 | 25.659 | 33.768 | −30.806 | 1.00 | 25.84 | O |
| ATOM | 1199 | OH2 | TIP | S | 43 | 11.670 | 34.591 | −31.049 | 1.00 | 24.04 | O |
| ATOM | 1200 | OH2 | TIP | S | 44 | 14.925 | 35.721 | −24.867 | 1.00 | 30.80 | O |
| ATOM | 1201 | OH2 | TIP | S | 45 | 14.241 | 43.369 | −22.021 | 1.00 | 28.12 | O |
| ATOM | 1202 | OH2 | TIP | S | 46 | −9.827 | 31.461 | −52.317 | 1.00 | 28.97 | O |
| ATOM | 1203 | OH2 | TIP | S | 47 | 8.506 | 31.396 | −33.265 | 1.00 | 29.75 | O |
| ATOM | 1204 | OH2 | TIP | S | 48 | 9.126 | 50.268 | −34.499 | 1.00 | 27.86 | O |
| ATOM | 1205 | OH2 | TIP | S | 49 | 16.265 | 29.480 | −24.811 | 1.00 | 36.40 | O |
| ATOM | 1206 | OH2 | TIP | S | 50 | −4.553 | 29.804 | −40.939 | 1.00 | 25.33 | O |
| ATOM | 1207 | OH2 | TIP | S | 51 | −0.925 | 24.231 | −41.854 | 1.00 | 28.51 | O |
| ATOM | 1208 | OH2 | TIP | S | 52 | 16.427 | 38.376 | −37.205 | 1.00 | 27.04 | O |
| ATOM | 1209 | OH2 | TIP | S | 53 | −15.913 | 27.381 | −43.840 | 1.00 | 32.30 | O |
| ATOM | 1210 | OH2 | TIP | S | 54 | 11.447 | 51.458 | −47.566 | 1.00 | 34.81 | O |
| ATOM | 1211 | OH2 | TIP | S | 55 | −20.434 | 33.968 | −42.557 | 1.00 | 36.37 | O |
| ATOM | 1212 | OH2 | TIP | S | 56 | 7.227 | 45.709 | −28.080 | 1.00 | 36.73 | O |
| ATOM | 1213 | OH2 | TIP | S | 57 | −22.166 | 32.059 | −45.749 | 1.00 | 40.49 | O |
| ATOM | 1214 | OH2 | TIP | S | 58 | 5.903 | 56.632 | −41.673 | 1.00 | 38.46 | O |
| ATOM | 1215 | OH2 | TIP | S | 59 | 18.150 | 45.535 | −36.175 | 1.00 | 37.77 | O |
| ATOM | 1216 | OH2 | TIP | S | 60 | 8.999 | 50.903 | −49.879 | 1.00 | 34.34 | O |
| ATOM | 1217 | OH2 | TIP | S | 61 | −6.750 | 26.968 | −47.098 | 1.00 | 34.69 | O |
| ATOM | 1218 | OH2 | TIP | S | 62 | −18.643 | 27.480 | −44.272 | 1.00 | 36.37 | O |
| ATOM | 1219 | OH2 | TIP | S | 63 | −16.785 | 27.296 | −40.673 | 1.00 | 35.65 | O |
| ATOM | 1220 | OH2 | TIP | S | 64 | 26.271 | 31.379 | −32.412 | 1.00 | 29.61 | O |
| ATOM | 1221 | OH2 | TIP | S | 65 | −8.590 | 32.103 | −28.199 | 1.00 | 38.48 | O |
| ATOM | 1222 | OH2 | TIP | S | 66 | −5.671 | 25.751 | −26.645 | 1.00 | 37.50 | O |
| ATOM | 1223 | OH2 | TIP | S | 67 | −11.451 | 28.222 | −48.868 | 1.00 | 29.77 | O |
| ATOM | 1224 | OH2 | TIP | S | 68 | 4.742 | 24.437 | −33.435 | 1.00 | 38.30 | O |
| ATOM | 1225 | OH2 | TIP | S | 69 | 18.117 | 34.332 | −21.727 | 1.00 | 41.86 | O |
| ATOM | 1226 | OH2 | TIP | S | 70 | 20.729 | 36.831 | −38.637 | 1.00 | 39.92 | O |
| ATOM | 1227 | OH2 | TIP | S | 71 | 14.479 | 48.924 | −39.242 | 1.00 | 39.55 | O |
| ATOM | 1228 | OH2 | TIP | S | 72 | −13.821 | 31.551 | −48.959 | 1.00 | 42.01 | O |
| ATOM | 1229 | OH2 | TIP | S | 73 | 15.590 | 47.645 | −35.246 | 1.00 | 31.94 | O |
| ATOM | 1230 | OH2 | TIP | S | 74 | 18.949 | 43.610 | −43.728 | 1.00 | 48.26 | O |
| ATOM | 1231 | OH2 | TIP | S | 75 | 18.097 | 37.022 | −38.528 | 1.00 | 42.07 | O |
| ATOM | 1232 | OH2 | TIP | S | 76 | 16.932 | 38.225 | −22.164 | 1.00 | 41.10 | O |
| ATOM | 1233 | OH2 | TIP | S | 77 | 11.113 | 32.143 | −47.202 | 1.00 | 24.30 | O |
| ATOM | 1234 | OH2 | TIP | S | 78 | −10.315 | 27.244 | −36.429 | 1.00 | 28.65 | O |
| ATOM | 1235 | OH2 | TIP | S | 79 | −1.936 | 24.324 | −33.480 | 1.00 | 31.75 | O |
| ATOM | 1236 | OH2 | TIP | S | 80 | 20.162 | 32.771 | −36.255 | 1.00 | 28.51 | O |
| ATOM | 1237 | OH2 | TIP | S | 81 | 13.968 | 36.749 | −43.678 | 1.00 | 26.92 | O |
| ATOM | 1238 | OH2 | TIP | S | 82 | 16.345 | 32.097 | −23.691 | 1.00 | 32.88 | O |
| ATOM | 1239 | OH2 | TIP | S | 83 | 22.857 | 26.185 | −29.537 | 1.00 | 32.84 | O |
| ATOM | 1240 | OH2 | TIP | S | 84 | 23.881 | 33.917 | −38.501 | 1.00 | 31.97 | O |
| ATOM | 1241 | OH2 | TIP | S | 85 | 22.284 | 34.994 | −36.916 | 1.00 | 29.53 | O |
| ATOM | 1242 | OH2 | TIP | S | 86 | 7.014 | 41.767 | −52.154 | 1.00 | 26.98 | O |
| ATOM | 1243 | OH2 | TIP | S | 87 | 0.812 | 23.273 | −39.923 | 1.00 | 37.48 | O |
| ATOM | 1244 | OH2 | TIP | S | 88 | 14.987 | 37.214 | −40.593 | 1.00 | 31.15 | O |
| ATOM | 1245 | OH2 | TIP | S | 89 | −6.352 | 25.077 | −36.593 | 1.00 | 33.45 | O |
| ATOM | 1246 | OH2 | TIP | S | 90 | 2.764 | 27.501 | −53.103 | 1.00 | 39.96 | O |
| ATOM | 1247 | OH2 | TIP | S | 91 | 23.931 | 27.318 | −33.585 | 1.00 | 39.51 | O |
| ATOM | 1248 | OH2 | TIP | S | 92 | 20.844 | 42.905 | −23.126 | 1.00 | 32.69 | O |
| ATOM | 1249 | OH2 | TIP | S | 93 | 28.707 | 31.189 | −31.305 | 1.00 | 39.90 | O |
| ATOM | 1250 | OH2 | TIP | S | 94 | 28.032 | 31.973 | −26.517 | 1.00 | 33.33 | O |
| ATOM | 1251 | OH2 | TIP | S | 95 | 5.125 | 29.269 | −28.102 | 1.00 | 41.09 | O |
| ATOM | 1252 | OH2 | TIP | S | 96 | 1.538 | 42.594 | −55.082 | 1.00 | 39.89 | O |
| ATOM | 1253 | OH2 | TIP | S | 97 | 5.142 | 33.882 | −28.543 | 1.00 | 29.65 | O |
| ATOM | 1254 | OH2 | TIP | S | 98 | 7.522 | 37.819 | −51.932 | 1.00 | 41.87 | O |
| ATOM | 1255 | OH2 | TIP | S | 99 | 16.995 | 47.970 | −43.596 | 1.00 | 45.54 | O |
| ATOM | 1256 | OH2 | TIP | S | 100 | 3.386 | 31.577 | −53.438 | 1.00 | 34.05 | O |
| ATOM | 1257 | OH2 | TIP | S | 101 | 12.358 | 25.908 | −32.630 | 1.00 | 43.09 | O |
| ATOM | 1258 | OH2 | TIP | S | 102 | −4.502 | 25.425 | −40.148 | 1.00 | 39.44 | O |
| ATOM | 1259 | OH2 | TIP | S | 103 | 2.326 | 23.321 | −36.267 | 1.00 | 36.24 | O |
| ATOM | 1260 | OH2 | TIP | S | 104 | −3.840 | 42.854 | −29.626 | 1.00 | 39.97 | O |
| ATOM | 1261 | OH2 | TIP | S | 105 | 11.109 | 34.728 | −28.256 | 1.00 | 39.02 | O |
| ATOM | 1262 | OH2 | TIP | S | 106 | −7.049 | 47.187 | −29.149 | 1.00 | 46.19 | O |
| ATOM | 1263 | OH2 | TIP | S | 107 | 8.696 | 50.838 | −31.752 | 1.00 | 38.26 | O |
| ATOM | 1264 | OH2 | TIP | S | 108 | 10.856 | 22.112 | −32.337 | 1.00 | 41.15 | O |
| ATOM | 1265 | OH2 | TIP | S | 109 | 1.975 | 22.619 | −47.835 | 1.00 | 49.21 | O |
| ATOM | 1266 | OH2 | TIP | S | 110 | −17.209 | 33.537 | −37.509 | 1.00 | 36.51 | O |
| ATOM | 1267 | OH2 | TIP | S | 111 | 11.659 | 21.076 | −49.137 | 1.00 | 53.87 | O |
| ATOM | 1268 | OH2 | TIP | S | 112 | 14.223 | 45.759 | −50.817 | 1.00 | 49.03 | O |
| ATOM | 1269 | OH2 | TIP | S | 113 | −15.401 | 31.484 | −33.345 | 1.00 | 40.60 | O |
| ATOM | 1270 | OH2 | TIP | S | 114 | 19.362 | 26.598 | −28.612 | 1.00 | 48.37 | O |
| ATOM | 1271 | OH2 | TIP | S | 115 | 7.431 | 49.367 | −29.820 | 1.00 | 41.73 | O |
| ATOM | 1272 | OH2 | TIP | S | 116 | 7.232 | 53.305 | −50.896 | 1.00 | 53.94 | O |
| ATOM | 1273 | OH2 | TIP | S | 117 | −3.020 | 46.604 | −30.113 | 1.00 | 48.72 | O |
| ATOM | 1274 | OH2 | TIP | S | 118 | 12.385 | 34.632 | −43.853 | 1.00 | 17.33 | O |
| ATOM | 1275 | OH2 | TIP | S | 119 | 14.154 | 36.582 | −37.742 | 1.00 | 24.77 | O |
| ATOM | 1276 | OH2 | TIP | S | 120 | 4.908 | 47.446 | −30.109 | 1.00 | 37.63 | O |
| ATOM | 1277 | OH2 | TIP | S | 121 | 6.222 | 31.655 | −29.384 | 1.00 | 25.17 | O |
| ATOM | 1278 | OH2 | TIP | S | 122 | 6.936 | 34.872 | −55.153 | 1.00 | 39.17 | O |
| ATOM | 1279 | OH2 | TIP | S | 123 | 20.217 | 30.372 | −37.400 | 1.00 | 38.24 | O |
| ATOM | 1280 | OH2 | TIP | S | 124 | 5.893 | 30.640 | −53.649 | 1.00 | 34.88 | O |
| ATOM | 1281 | OH2 | TIP | S | 125 | 6.588 | 37.422 | −54.426 | 1.00 | 36.59 | O |
| ATOM | 1282 | OH2 | TIP | S | 126 | −0.094 | 22.674 | −34.881 | 1.00 | 44.41 | O |
| ATOM | 1283 | OH2 | TIP | S | 127 | 10.378 | 27.577 | −32.359 | 1.00 | 38.50 | O |
| ATOM | 1284 | OH2 | TIP | S | 128 | 14.540 | 34.402 | −40.444 | 1.00 | 39.48 | O |
| ATOM | 1285 | OH2 | TIP | S | 129 | 4.496 | 50.788 | −29.182 | 1.00 | 45.05 | O |
| ATOM | 1286 | OH2 | TIP | S | 130 | 6.408 | 22.573 | −35.459 | 1.00 | 50.43 | O |
| ATOM | 1287 | OH2 | TIP | S | 131 | −12.050 | 32.985 | −51.422 | 1.00 | 48.91 | O |
| ATOM | 1288 | OH2 | TIP | S | 132 | −17.040 | 36.167 | −37.739 | 1.00 | 37.60 | O |
| ATOM | 1289 | OH2 | TIP | S | 133 | −13.777 | 36.722 | −31.648 | 1.00 | 43.95 | O |
| ATOM | 1290 | OH2 | TIP | S | 134 | −16.049 | 26.086 | −37.549 | 1.00 | 51.05 | O |
| ATOM | 1291 | OH2 | TIP | S | 135 | 27.742 | 35.447 | −30.568 | 1.00 | 51.93 | O |
| ATOM | 1292 | OH2 | TIP | S | 136 | 24.997 | 39.617 | −38.777 | 1.00 | 47.11 | O |
| ATOM | 1293 | OH2 | TIP | S | 137 | 0.185 | 35.562 | −25.783 | 1.00 | 43.00 | O |
| ATOM | 1294 | OH2 | TIP | S | 138 | 10.480 | 55.080 | −33.160 | 1.00 | 52.31 | O |
| ATOM | 1295 | OH2 | TIP | S | 139 | 14.051 | 20.534 | −40.043 | 1.00 | 49.55 | O |
| ATOM | 1296 | OH2 | TIP | S | 140 | 16.465 | 49.414 | −47.240 | 1.00 | 48.94 | O |
| ATOM | 1297 | OH2 | TIP | S | 141 | 8.955 | 32.884 | −31.175 | 1.00 | 51.15 | O |
| ATOM | 1298 | OH2 | TIP | S | 142 | −22.281 | 28.847 | −45.057 | 1.00 | 33.26 | O |
| ATOM | 1299 | OH2 | TIP | S | 143 | −15.182 | 33.251 | −31.347 | 1.00 | 51.94 | O |
| ATOM | 1300 | OH2 | TIP | S | 144 | 17.310 | 27.467 | −31.346 | 1.00 | 47.32 | O |
| ATOM | 1301 | OH2 | TIP | S | 145 | 12.427 | 30.220 | −21.690 | 1.00 | 58.13 | O |
| ATOM | 1302 | OH2 | TIP | S | 146 | −14.089 | 24.436 | −36.330 | 1.00 | 46.84 | O |
| ATOM | 1303 | OH2 | TIP | S | 147 | 12.987 | 49.523 | −26.982 | 1.00 | 52.33 | O |
| ATOM | 1304 | OH2 | TIP | S | 148 | 23.820 | 28.870 | −19.392 | 1.00 | 39.18 | O |
| ATOM | 1305 | OH2 | TIP | S | 149 | 0.915 | 27.826 | −28.160 | 1.00 | 39.66 | O |
| ATOM | 1306 | OH2 | TIP | S | 150 | 18.428 | 50.726 | −30.387 | 1.00 | 45.16 | O |
| ATOM | 1307 | OH2 | TIP | S | 151 | −4.284 | 23.686 | −34.753 | 1.00 | 45.17 | O |
| ATOM | 1308 | C2 | L39 | C | 1 | −2.384 | 44.155 | −42.022 | 1.00 | 54.83 | C |
| ATOM | 1309 | C3 | L39 | C | 1 | −3.652 | 43.539 | −42.215 | 1.00 | 55.77 | C |
| ATOM | 1310 | C4 | L39 | C | 1 | −4.432 | 43.115 | −41.096 | 1.00 | 56.98 | C |
| ATOM | 1311 | N7 | L39 | C | 1 | −5.646 | 42.528 | −41.322 | 1.00 | 59.32 | N |
| ATOM | 1312 | C11 | L39 | C | 1 | −6.837 | 43.079 | −41.153 | 1.00 | 60.27 | C |
| ATOM | 1313 | O13 | L39 | C | 1 | −7.095 | 44.327 | −40.722 | 1.00 | 61.09 | O |
| ATOM | 1314 | C10 | L39 | C | 1 | −7.807 | 42.215 | −41.473 | 1.00 | 60.22 | C |
| ATOM | 1315 | C9 | L39 | C | 1 | −7.073 | 41.014 | −41.877 | 1.00 | 59.77 | C |
| ATOM | 1316 | O12 | L39 | C | 1 | −7.656 | 39.884 | −42.283 | 1.00 | 60.30 | O |
| ATOM | 1317 | N8 | L39 | C | 1 | −5.834 | 41.263 | −41.767 | 1.00 | 59.43 | N |
| ATOM | 1318 | C5 | L39 | C | 1 | −3.920 | 43.317 | −39.775 | 1.00 | 55.83 | C |
| ATOM | 1319 | C6 | L39 | C | 1 | −2.649 | 43.934 | −39.584 | 1.00 | 54.82 | C |
| ATOM | 1320 | C1 | L39 | C | 1 | −1.882 | 44.353 | −40.707 | 1.00 | 53.88 | C |
| ATOM | 1321 | O1 | PGA | B | 180 | 13.247 | 35.091 | −35.643 | 1.00 | 21.80 | O |
| ATOM | 1322 | C2 | PGA | B | 180 | 11.924 | 34.755 | −35.651 | 1.00 | 24.34 | C |
| ATOM | 1323 | C3 | PGA | B | 180 | 11.610 | 33.748 | −34.528 | 1.00 | 24.78 | C |
| ATOM | 1324 | O4 | PGA | B | 180 | 12.151 | 32.559 | −34.792 | 1.00 | 22.53 | O |
| ATOM | 1325 | C5 | PGA | B | 180 | 11.898 | 31.671 | −33.831 | 1.00 | 22.49 | C |
| ATOM | 1326 | C6 | PGA | B | 180 | 12.544 | 30.309 | −34.175 | 1.00 | 22.18 | C |
| ATOM | 1327 | O7 | PGA | B | 180 | 13.877 | 30.454 | −34.380 | 1.00 | 24.17 | O |
| ATOM | 1328 | C8 | PGA | B | 180 | 14.479 | 29.277 | −34.663 | 1.00 | 24.81 | C |
| ATOM | 1329 | C9 | PGA | B | 180 | 15.994 | 29.488 | −34.895 | 1.00 | 24.23 | C |
| ATOM | 1330 | O10 | PGA | B | 180 | 16.536 | 30.250 | −33.902 | 1.00 | 27.21 | O |
| ATOM | 1331 | C11 | PGA | B | 180 | 17.856 | 30.454 | −34.084 | 1.00 | 28.90 | C |
| ATOM | 1332 | C12 | PGA | B | 180 | 18.444 | 31.209 | −32.871 | 1.00 | 27.39 | C |
| ATOM | 1333 | O13 | PGA | B | 180 | 18.015 | 30.654 | −31.729 | 1.00 | 30.53 | O |
| ATOM | 1334 | C14 | PGA | B | 180 | 18.516 | 31.303 | −30.677 | 1.00 | 30.71 | C |
| ATOM | 1335 | C15 | PGA | B | 180 | 18.002 | 30.670 | −29.361 | 1.00 | 33.11 | C |
| ATOM | 1336 | O16 | PGA | B | 180 | 16.748 | 30.193 | −29.474 | 1.00 | 37.48 | O |
| ATOM | 1337 | C17 | PGA | B | 180 | 15.996 | 30.910 | −28.700 | 1.00 | 39.27 | C |
| ATOM | 1338 | C18 | PGA | B | 180 | 14.536 | 30.484 | −28.727 | 1.00 | 42.26 | C |
| ATOM | 1339 | O19 | PGA | B | 180 | 13.881 | 31.280 | −27.919 | 1.00 | 43.72 | |

Pin1 R14A/Citrate/Glycerol Interactions

The crystal structure of Pin1 crystallized in the presence of citrate and glycerol was determined (the coordinate set is set forth in Table 6). The citrate molecule binds in the phosphate pocket, making interactions with Lys63 and Arg69. Glycerol binds in the hydrophobic pocket, hydrogen bonding with Gln131.

TABLE 6
Set of atomic coordinates of Pin1 R14A with glycerol and citrate bound in the active site.

| | | | | | | | | | | | |
|---|---|---|---|---|---|---|---|---|---|---|---|
| ATOM | 1 | CB | LYS | | 6 | 55.445 | 2.509 | −80.998 | 1.00 | 98.16 | C |
| ATOM | 2 | CG | LYS | | 6 | 54.950 | 2.240 | −79.586 | 1.00 | 98.85 | C |
| ATOM | 3 | CD | LYS | | 6 | 56.005 | 2.620 | −78.561 | 1.00 | 99.89 | C |
| ATOM | 4 | CE | LYS | | 6 | 55.563 | 2.261 | −77.154 | 1.00 | 100.16 | C |
| ATOM | 5 | NZ | LYS | | 6 | 56.604 | 2.603 | −76.147 | 1.00 | 100.86 | N |
| ATOM | 6 | C | LYS | | 6 | 53.285 | 2.961 | −82.191 | 1.00 | 81.57 | C |
| ATOM | 7 | O | LYS | | 6 | 52.898 | 3.595 | −81.208 | 1.00 | 82.16 | O |
| ATOM | 8 | N | LYS | | 6 | 55.211 | 1.998 | −83.407 | 1.00 | 82.24 | N |
| ATOM | 9 | CA | LYS | | 6 | 54.496 | 2.033 | −82.101 | 1.00 | 82.23 | C |
| ATOM | 10 | N | LEU | | 7 | 52.707 | 3.027 | −83.390 | 1.00 | 65.96 | N |
| ATOM | 11 | CA | LEU | | 7 | 51.528 | 3.839 | −83.692 | 1.00 | 64.20 | C |
| ATOM | 12 | CB | LEU | | 7 | 50.571 | 3.887 | −82.490 | 1.00 | 54.94 | C |
| ATOM | 13 | CG | LEU | | 7 | 49.984 | 2.559 | −82.002 | 1.00 | 54.86 | C |
| ATOM | 14 | CD1 | LEU | | 7 | 48.929 | 2.824 | −80.938 | 1.00 | 55.00 | C |
| ATOM | 15 | CD2 | LEU | | 7 | 49.375 | 1.810 | −83.173 | 1.00 | 54.83 | C |
| ATOM | 16 | C | LEU | | 7 | 51.835 | 5.262 | −84.148 | 1.00 | 62.41 | C |
| ATOM | 17 | O | LEU | | 7 | 52.450 | 6.044 | −83.424 | 1.00 | 62.15 | O |
| ATOM | 18 | N | PRO | | 8 | 51.400 | 5.609 | −85.369 | 1.00 | 59.55 | N |
| ATOM | 19 | CD | PRO | | 8 | 50.791 | 4.670 | −86.324 | 1.00 | 56.96 | C |
| ATOM | 20 | CA | PRO | | 8 | 51.585 | 6.923 | −85.990 | 1.00 | 57.77 | C |
| ATOM | 21 | CB | PRO | | 8 | 50.850 | 6.775 | −87.320 | 1.00 | 55.92 | C |
| ATOM | 22 | CG | PRO | | 8 | 51.068 | 5.344 | −87.650 | 1.00 | 56.52 | C |
| ATOM | 23 | C | PRO | | 8 | 51.006 | 8.042 | −85.125 | 1.00 | 56.10 | C |
| ATOM | 24 | O | PRO | | 8 | 50.390 | 7.785 | −84.086 | 1.00 | 55.67 | O |
| ATOM | 25 | N | PRO | | 9 | 51.192 | 9.301 | −85.550 | 1.00 | 53.27 | N |
| ATOM | 26 | CD | PRO | | 9 | 51.939 | 9.704 | −86.755 | 1.00 | 43.20 | C |
| ATOM | 27 | CA | PRO | | 9 | 50.702 | 10.477 | −84.827 | 1.00 | 49.97 | C |
| ATOM | 28 | CB | PRO | | 9 | 51.038 | 11.630 | −85.774 | 1.00 | 41.41 | C |
| ATOM | 29 | CG | PRO | | 9 | 52.285 | 11.147 | −86.451 | 1.00 | 43.41 | C |
| ATOM | 30 | C | PRO | | 9 | 49.222 | 10.449 | −84.475 | 1.00 | 44.98 | C |
| ATOM | 31 | O | PRO | | 9 | 48.366 | 10.388 | −85.359 | 1.00 | 45.29 | O |
| ATOM | 32 | N | GLY | | 10 | 48.933 | 10.496 | −83.177 | 1.00 | 43.90 | N |
| ATOM | 33 | CA | GLY | | 10 | 47.553 | 10.516 | −82.724 | 1.00 | 37.61 | C |
| ATOM | 34 | C | GLY | | 10 | 46.870 | 9.195 | −82.406 | 1.00 | 32.29 | C |
| ATOM | 35 | O | GLY | | 10 | 45.769 | 9.200 | −81.858 | 1.00 | 28.79 | O |
| ATOM | 36 | N | TRP | | 11 | 47.501 | 8.074 | −82.739 | 1.00 | 43.59 | N |
| ATOM | 37 | CA | TRP | | 11 | 46.898 | 6.769 | −82.481 | 1.00 | 42.04 | C |
| ATOM | 38 | CB | TRP | | 11 | 47.348 | 5.747 | −83.531 | 1.00 | 33.82 | C |
| ATOM | 39 | CG | TRP | | 11 | 46.829 | 6.004 | −84.911 | 1.00 | 32.29 | C |
| ATOM | 40 | CD2 | TRP | | 11 | 45.526 | 5.669 | −85.417 | 1.00 | 31.33 | C |
| ATOM | 41 | CE2 | TRP | | 11 | 45.484 | 6.081 | −86.766 | 1.00 | 30.36 | C |
| ATOM | 42 | CE3 | TRP | | 11 | 44.396 | 5.062 | −84.860 | 1.00 | 28.35 | C |
| ATOM | 43 | CD1 | TRP | | 11 | 47.498 | 6.590 | −85.940 | 1.00 | 32.31 | C |
| ATOM | 44 | NE1 | TRP | | 11 | 46.699 | 6.640 | −87.059 | 1.00 | 31.61 | N |
| ATOM | 45 | CZ2 | TRP | | 11 | 44.358 | 5.906 | −87.570 | 1.00 | 28.84 | C |
| ATOM | 46 | CZ3 | TRP | | 11 | 43.271 | 4.889 | −85.662 | 1.00 | 27.55 | C |
| ATOM | 47 | CH2 | TRP | | 11 | 43.262 | 5.309 | −87.003 | 1.00 | 28.50 | C |
| ATOM | 48 | C | TRP | | 11 | 47.193 | 6.198 | −81.101 | 1.00 | 41.92 | C |
| ATOM | 49 | O | TRP | | 11 | 48.302 | 6.320 | −80.584 | 1.00 | 41.10 | O |
| ATOM | 50 | N | GLU | | 12 | 46.184 | 5.565 | −80.515 | 1.00 | 34.93 | N |
| ATOM | 51 | CA | GLU | | 12 | 46.313 | 4.929 | −79.209 | 1.00 | 33.99 | C |
| ATOM | 52 | CB | GLU | | 12 | 45.706 | 5.807 | −78.109 | 1.00 | 69.08 | C |
| ATOM | 53 | CG | GLU | | 12 | 44.192 | 5.964 | −78.208 | 1.00 | 71.07 | C |
| ATOM | 54 | CD | GLU | | 12 | 43.579 | 6.614 | −76.978 | 1.00 | 72.99 | C |
| ATOM | 55 | OE1 | GLU | | 12 | 43.981 | 7.745 | −76.630 | 1.00 | 72.29 | O |
| ATOM | 56 | OE2 | GLU | | 12 | 42.691 | 5.991 | −76.359 | 1.00 | 73.29 | O |
| ATOM | 57 | C | GLU | | 12 | 45.560 | 3.598 | −79.265 | 1.00 | 32.93 | C |
| ATOM | 58 | O | GLU | | 12 | 44.650 | 3.420 | −80.076 | 1.00 | 31.05 | O |
| ATOM | 59 | N | LYS | | 13 | 45.940 | 2.664 | −78.404 | 1.00 | 38.02 | N |
| ATOM | 60 | CA | LYS | | 13 | 45.275 | 1.374 | −78.364 | 1.00 | 39.73 | C |
| ATOM | 61 | CB | LYS | | 13 | 46.208 | 0.321 | −77.763 | 1.00 | 65.15 | C |
| ATOM | 62 | CG | LYS | | 13 | 45.730 | −1.110 | −77.923 | 1.00 | 68.38 | C |
| ATOM | 63 | CD | LYS | | 13 | 46.538 | −2.055 | −77.042 | 1.00 | 70.78 | C |
| ATOM | 64 | CE | LYS | | 13 | 48.032 | −1.962 | −77.330 | 1.00 | 71.66 | C |
| ATOM | 65 | NZ | LYS | | 13 | 48.848 | −2.730 | −76.337 | 1.00 | 72.97 | N |
| ATOM | 66 | C | LYS | | 13 | 44.046 | 1.550 | −77.483 | 1.00 | 39.24 | C |
| ATOM | 67 | O | LYS | | 13 | 44.083 | 2.281 | −76.494 | 1.00 | 38.03 | O |
| ATOM | 68 | N | ALA | | 14 | 42.950 | 0.899 | −77.848 | 1.00 | 33.96 | N |
| ATOM | 69 | CA | ALA | | 14 | 41.730 | 1.006 | −77.064 | 1.00 | 34.14 | C |
| ATOM | 70 | CB | ALA | | 14 | 40.778 | 2.002 | −77.707 | 1.00 | 30.95 | C |
| ATOM | 71 | C | ALA | | 14 | 41.045 | −0.343 | −76.903 | 1.00 | 33.37 | C |
| ATOM | 72 | O | ALA | | 14 | 41.356 | −1.310 | −77.602 | 1.00 | 31.73 | O |
| ATOM | 73 | N | MET | | 15 | 40.101 | −0.390 | −75.977 | 1.00 | 34.01 | N |
| ATOM | 74 | CA | MET | | 15 | 39.360 | −1.606 | −75.696 | 1.00 | 34.17 | C |
| ATOM | 75 | CB | MET | | 15 | 39.486 | −1.946 | −74.208 | 1.00 | 48.76 | C |
| ATOM | 76 | CG | MET | | 15 | 38.650 | −3.131 | −73.784 | 1.00 | 51.61 | C |
| ATOM | 77 | SD | MET | | 15 | 39.016 | −4.532 | −74.824 | 1.00 | 55.30 | S |
| ATOM | 78 | CE | MET | | 15 | 40.517 | −5.143 | −74.033 | 1.00 | 53.03 | C |
| ATOM | 79 | C | MET | | 15 | 37.888 | −1.459 | −76.068 | 1.00 | 33.51 | C |
| ATOM | 80 | O | MET | | 15 | 37.201 | −0.579 | −75.564 | 1.00 | 31.33 | O |
| ATOM | 81 | N | SER | | 16 | 37.409 | −2.319 | −76.958 | 1.00 | 30.37 | N |
| ATOM | 82 | CA | SER | | 16 | 36.008 | −2.273 | −77.365 | 1.00 | 29.84 | C |
| ATOM | 83 | CB | SER | | 16 | 35.761 | −3.206 | −78.546 | 1.00 | 34.48 | C |
| ATOM | 84 | OG | SER | | 16 | 34.369 | −3.381 | −78.743 | 1.00 | 35.56 | O |
| ATOM | 85 | C | SER | | 16 | 35.093 | −2.690 | −76.215 | 1.00 | 30.49 | C |
| ATOM | 86 | O | SER | | 16 | 35.329 | −3.711 | −75.571 | 1.00 | 29.50 | O |
| ATOM | 87 | N | ARG | | 17 | 34.047 | −1.905 | −75.973 | 1.00 | 37.72 | N |
| ATOM | 88 | CA | ARG | | 17 | 33.089 | −2.189 | −74.902 | 1.00 | 40.43 | C |
| ATOM | 89 | CB | ARG | | 17 | 32.196 | −0.971 | −74.656 | 1.00 | 48.73 | C |
| ATOM | 90 | CG | ARG | | 17 | 32.922 | 0.209 | −74.071 | 1.00 | 51.66 | C |
| ATOM | 91 | CD | ARG | | 17 | 33.484 | −0.140 | −72.712 | 1.00 | 52.09 | C |
| ATOM | 92 | NE | ARG | | 17 | 34.421 | 0.874 | −72.246 | 1.00 | 54.91 | N |
| ATOM | 93 | CZ | ARG | | 17 | 34.088 | 2.128 | −71.959 | 1.00 | 55.39 | C |
| ATOM | 94 | NH1 | ARG | | 17 | 32.831 | 2.536 | −72.084 | 1.00 | 55.18 | N |
| ATOM | 95 | NH2 | ARG | | 17 | 35.022 | 2.976 | −71.558 | 1.00 | 55.61 | N |
| ATOM | 96 | C | ARG | | 17 | 32.195 | −3.391 | −75.195 | 1.00 | 41.27 | C |
| ATOM | 97 | O | ARG | | 17 | 31.874 | −4.170 | −74.298 | 1.00 | 40.34 | O |
| ATOM | 98 | N | SER | | 18 | 31.793 | −3.528 | −76.452 | 1.00 | 38.74 | N |
| ATOM | 99 | CA | SER | | 18 | 30.916 | −4.614 | −76.870 | 1.00 | 41.26 | C |
| ATOM | 100 | CB | SER | | 18 | 30.303 | −4.290 | −78.236 | 1.00 | 41.67 | C |
| ATOM | 101 | OG | SER | | 18 | 29.555 | −3.092 | −78.197 | 1.00 | 46.84 | O |
| ATOM | 102 | C | SER | | 18 | 31.589 | −5.977 | −76.961 | 1.00 | 41.23 | C |
| ATOM | 103 | O | SER | | 18 | 31.176 | −6.934 | −76.303 | 1.00 | 43.80 | O |
| ATOM | 104 | N | SER | | 19 | 32.632 | −6.051 | −77.780 | 1.00 | 30.71 | N |
| ATOM | 105 | CA | SER | | 19 | 33.344 | −7.298 | −78.023 | 1.00 | 30.26 | C |
| ATOM | 106 | CB | SER | | 19 | 33.796 | −7.347 | −79.487 | 1.00 | 42.73 | C |
| ATOM | 107 | OG | SER | | 19 | 34.735 | −6.319 | −79.762 | 1.00 | 44.23 | O |
| ATOM | 108 | C | SER | | 19 | 34.544 | −7.597 | −77.140 | 1.00 | 29.87 | C |
| ATOM | 109 | O | SER | | 19 | 34.920 | −8.757 | −76.993 | 1.00 | 29.63 | O |
| ATOM | 110 | N | GLY | | 20 | 35.151 | −6.566 | −76.562 | 1.00 | 30.24 | N |
| ATOM | 111 | CA | GLY | | 20 | 36.322 | −6.794 | −75.732 | 1.00 | 28.21 | C |
| ATOM | 112 | C | GLY | | 20 | 37.576 | −6.918 | −76.588 | 1.00 | 27.03 | C |
| ATOM | 113 | O | GLY | | 20 | 38.673 | −7.140 | −76.087 | 1.00 | 26.49 | O |
| ATOM | 114 | N | ARG | | 21 | 37.415 | −6.784 | −77.898 | 1.00 | 31.19 | N |
| ATOM | 115 | CA | ARG | | 21 | 38.549 | −6.866 | −78.807 | 1.00 | 30.60 | C |
| ATOM | 116 | CB | ARG | | 21 | 38.063 | −7.196 | −80.219 | 1.00 | 45.79 | C |
| ATOM | 117 | CG | ARG | | 21 | 38.946 | −8.152 | −81.013 | 1.00 | 50.43 | C |
| ATOM | 118 | CD | ARG | | 21 | 38.498 | −8.167 | −82.465 | 1.00 | 52.82 | C |
| ATOM | 119 | NE | ARG | | 21 | 37.045 | −8.064 | −82.552 | 1.00 | 52.89 | N |
| ATOM | 120 | CZ | ARG | | 21 | 36.391 | −7.481 | −83.551 | 1.00 | 52.85 | C |
| ATOM | 121 | NH1 | ARG | | 21 | 37.055 | −6.943 | −84.569 | 1.00 | 51.07 | N |
| ATOM | 122 | NH2 | ARG | | 21 | 35.067 | −7.411 | −83.512 | 1.00 | 54.89 | N |
| ATOM | 123 | C | ARG | | 21 | 39.216 | −5.495 | −78.812 | 1.00 | 27.77 | C |
| ATOM | 124 | O | ARG | | 21 | 38.540 | −4.466 | −78.698 | 1.00 | 24.90 | O |
| ATOM | 125 | N | VAL | | 22 | 40.535 | −5.471 | −78.937 | 1.00 | 27.42 | N |
| ATOM | 126 | CA | VAL | | 22 | 41.248 | −4.201 | −78.971 | 1.00 | 26.89 | C |
| ATOM | 127 | CB | VAL | | 22 | 42.777 | −4.396 | −78.768 | 1.00 | 36.69 | C |
| ATOM | 128 | CG1 | VAL | | 22 | 43.047 | −5.169 | −77.480 | 1.00 | 39.71 | C |
| ATOM | 129 | CG2 | VAL | | 22 | 43.373 | −5.134 | −79.959 | 1.00 | 38.47 | C |
| ATOM | 130 | C | VAL | | 22 | 41.030 | −3.537 | −80.339 | 1.00 | 24.89 | C |
| ATOM | 131 | O | VAL | | 22 | 40.745 | −4.207 | −81.332 | 1.00 | 23.24 | O |
| ATOM | 132 | N | TYR | | 23 | 41.137 | −2.217 | −80.380 | 1.00 | 27.14 | N |
| ATOM | 133 | CA | TYR | | 23 | 41.008 | −1.487 | −81.635 | 1.00 | 25.75 | C |
| ATOM | 134 | CB | TYR | | 23 | 39.553 | −1.092 | −81.905 | 1.00 | 25.74 | C |
| ATOM | 135 | CG | TYR | | 23 | 38.968 | −0.035 | −80.996 | 1.00 | 27.50 | C |
| ATOM | 136 | CD1 | TYR | | 23 | 38.850 | 1.287 | −81.423 | 1.00 | 28.08 | C |
| ATOM | 137 | CE1 | TYR | | 23 | 38.263 | 2.249 | −80.624 | 1.00 | 27.02 | C |
| ATOM | 138 | CD2 | TYR | | 23 | 38.486 | −0.365 | −79.735 | 1.00 | 28.62 | C |
| ATOM | 139 | CE2 | TYR | | 23 | 37.898 | 0.590 | −78.921 | 1.00 | 30.69 | C |
| ATOM | 140 | CZ | TYR | | 23 | 37.786 | 1.897 | −79.372 | 1.00 | 29.96 | C |
| ATOM | 141 | OH | TYR | | 23 | 37.187 | 2.845 | −78.574 | 1.00 | 30.52 | O |
| ATOM | 142 | C | TYR | | 23 | 41.898 | −0.268 | −81.504 | 1.00 | 26.35 | C |
| ATOM | 143 | O | TYR | | 23 | 42.559 | −0.101 | −80.483 | 1.00 | 26.15 | O |
| ATOM | 144 | N | TYR | | 24 | 41.943 | 0.571 | −82.531 | 1.00 | 26.24 | N |
| ATOM | 145 | CA | TYR | | 24 | 42.787 | 1.755 | −82.476 | 1.00 | 26.60 | C |
| ATOM | 146 | CB | TYR | | 24 | 43.939 | 1.628 | −83.478 | 1.00 | 30.68 | C |
| ATOM | 147 | CG | TYR | | 24 | 44.780 | 0.393 | −83.249 | 1.00 | 35.00 | C |
| ATOM | 148 | CD1 | TYR | | 24 | 44.347 | −0.858 | −83.681 | 1.00 | 37.19 | C |
| ATOM | 149 | CE1 | TYR | | 24 | 45.082 | −2.002 | −83.411 | 1.00 | 38.80 | C |
| ATOM | 150 | CD2 | TYR | | 24 | 45.979 | 0.465 | −82.544 | 1.00 | 37.77 | C |
| ATOM | 151 | CE2 | TYR | | 24 | 46.721 | −0.671 | −82.270 | 1.00 | 38.67 | C |
| ATOM | 152 | CZ | TYR | | 24 | 46.268 | −1.901 | −82.704 | 1.00 | 40.68 | C |
| ATOM | 153 | OH | TYR | | 24 | 46.997 | −3.034 | −82.422 | 1.00 | 42.21 | O |
| ATOM | 154 | C | TYR | | 24 | 42.000 | 3.026 | −82.732 | 1.00 | 25.90 | C |
| ATOM | 155 | O | TYR | | 24 | 41.139 | 3.081 | −83.611 | 1.00 | 23.89 | O |
| ATOM | 156 | N | PHE | | 25 | 42.315 | 4.048 | −81.950 | 1.00 | 27.07 | N |
| ATOM | 157 | CA | PHE | | 25 | 41.653 | 5.337 | −82.038 | 1.00 | 27.62 | C |
| ATOM | 158 | CB | PHE | | 25 | 40.857 | 5.564 | −80.753 | 1.00 | 31.38 | C |
| ATOM | 159 | CG | PHE | | 25 | 40.293 | 6.939 | −80.618 | 1.00 | 32.02 | C |
| ATOM | 160 | CD1 | PHE | | 25 | 39.220 | 7.350 | −81.402 | 1.00 | 32.11 | C |
| ATOM | 161 | CD2 | PHE | | 25 | 40.827 | 7.829 | −79.696 | 1.00 | 32.08 | C |
| ATOM | 162 | CE1 | PHE | | 25 | 38.686 | 8.624 | −81.264 | 1.00 | 31.38 | C |
| ATOM | 163 | CE2 | PHE | | 25 | 40.299 | 9.103 | −79.554 | 1.00 | 32.00 | C |
| ATOM | 164 | CZ | PHE | | 25 | 39.225 | 9.500 | −80.342 | 1.00 | 32.52 | C |
| ATOM | 165 | C | PHE | | 25 | 42.694 | 6.446 | −82.213 | 1.00 | 28.03 | C |
| ATOM | 166 | O | PHE | | 25 | 43.786 | 6.379 | −81.647 | 1.00 | 28.18 | O |
| ATOM | 167 | N | ASN | | 26 | 42.349 | 7.456 | −83.003 | 1.00 | 27.36 | N |
| ATOM | 168 | CA | ASN | | 26 | 43.230 | 8.596 | −83.246 | 1.00 | 27.09 | C |
| ATOM | 169 | CB | ASN | | 26 | 43.482 | 8.767 | −84.752 | 1.00 | 24.37 | C |
| ATOM | 170 | CG | ASN | | 26 | 44.564 | 9.801 | −85.053 | 1.00 | 22.43 | C |
| ATOM | 171 | OD1 | ASN | | 26 | 44.413 | 10.983 | −84.743 | 1.00 | 22.07 | O |
| ATOM | 172 | ND2 | ASN | | 26 | 45.663 | 9.350 | −85.660 | 1.00 | 22.99 | N |
| ATOM | 173 | C | ASN | | 26 | 42.515 | 9.822 | −82.676 | 1.00 | 24.70 | C |
| ATOM | 174 | O | ASN | | 26 | 41.445 | 10.204 | −83.154 | 1.00 | 24.38 | O |
| ATOM | 175 | N | HIS | | 27 | 43.099 | 10.431 | −81.648 | 1.00 | 20.16 | N |
| ATOM | 176 | CA | HIS | | 27 | 42.475 | 11.583 | −81.005 | 1.00 | 19.80 | C |
| ATOM | 177 | CB | HIS | | 27 | 43.014 | 11.749 | −79.577 | 1.00 | 21.98 | C |
| ATOM | 178 | CG | HIS | | 27 | 44.467 | 12.091 | −79.516 | 1.00 | 24.66 | C |
| ATOM | 179 | CD2 | HIS | | 27 | 45.547 | 11.334 | −79.215 | 1.00 | 25.19 | C |
| ATOM | 180 | ND1 | HIS | | 27 | 44.946 | 13.352 | −79.801 | 1.00 | 27.16 | N |
| ATOM | 181 | CE1 | HIS | | 27 | 46.261 | 13.357 | −79.677 | 1.00 | 26.43 | C |
| ATOM | 182 | NE2 | HIS | | 27 | 46.651 | 12.145 | −79.323 | 1.00 | 29.10 | N |
| ATOM | 183 | C | HIS | | 27 | 42.606 | 12.899 | −81.773 | 1.00 | 20.10 | C |
| ATOM | 184 | O | HIS | | 27 | 42.059 | 13.920 | −81.356 | 1.00 | 20.52 | O |
| ATOM | 185 | N | ILE | | 28 | 43.321 | 12.872 | −82.890 | 1.00 | 30.65 | N |
| ATOM | 186 | CA | ILE | | 28 | 43.475 | 14.066 | −83.706 | 1.00 | 31.11 | C |
| ATOM | 187 | CB | ILE | | 28 | 44.887 | 14.145 | −84.339 | 1.00 | 27.16 | C |
| ATOM | 188 | CG2 | ILE | | 28 | 45.013 | 15.413 | −85.177 | 1.00 | 26.73 | C |
| ATOM | 189 | CG1 | ILE | | 28 | 45.951 | 14.155 | −83.238 | 1.00 | 26.99 | C |
| ATOM | 190 | CD1 | ILE | | 28 | 47.379 | 14.051 | −83.751 | 1.00 | 27.95 | C |
| ATOM | 191 | C | ILE | | 28 | 42.417 | 14.040 | −84.815 | 1.00 | 30.08 | C |
| ATOM | 192 | O | ILE | | 28 | 41.760 | 15.042 | −85.066 | 1.00 | 29.99 | O |
| ATOM | 193 | N | THR | | 29 | 42.232 | 12.885 | −85.452 | 1.00 | 24.76 | N |
| ATOM | 194 | CA | THR | | 29 | 41.251 | 12.758 | −86.533 | 1.00 | 24.81 | C |
| ATOM | 195 | CB | THR | | 29 | 41.778 | 11.867 | −87.653 | 1.00 | 22.03 | C |
| ATOM | 196 | OG1 | THR | | 29 | 41.859 | 10.520 | −87.172 | 1.00 | 18.99 | O |
| ATOM | 197 | CG2 | THR | | 29 | 43.167 | 12.315 | −88.100 | 1.00 | 21.61 | C |
| ATOM | 198 | C | THR | | 29 | 39.916 | 12.153 | −86.092 | 1.00 | 24.90 | C |
| ATOM | 199 | O | THR | | 29 | 38.935 | 12.215 | −86.834 | 1.00 | 23.10 | O |
| ATOM | 200 | N | ASN | | 30 | 39.891 | 11.566 | −84.895 | 1.00 | 27.70 | N |
| ATOM | 201 | CA | ASN | | 30 | 38.701 | 10.907 | −84.354 | 1.00 | 26.96 | C |
| ATOM | 202 | CB | ASN | | 30 | 37.507 | 11.875 | −84.317 | 1.00 | 20.48 | C |
| ATOM | 203 | CG | ASN | | 30 | 37.499 | 12.735 | −83.050 | 1.00 | 20.74 | C |
| ATOM | 204 | OD1 | ASN | | 30 | 36.882 | 13.801 | −82.998 | 1.00 | 21.01 | O |
| ATOM | 205 | ND2 | ASN | | 30 | 38.180 | 12.260 | −82.025 | 1.00 | 19.77 | N |
| ATOM | 206 | C | ASN | | 30 | 38.352 | 9.632 | −85.138 | 1.00 | 27.36 | C |
| ATOM | 207 | O | ASN | | 30 | 37.208 | 9.161 | −85.128 | 1.00 | 27.42 | O |
| ATOM | 208 | N | ALA | | 31 | 39.345 | 9.074 | −85.822 | 1.00 | 23.88 | N |
| ATOM | 209 | CA | ALA | | 31 | 39.126 | 7.844 | −86.567 | 1.00 | 24.36 | C |
| ATOM | 210 | CB | ALA | | 31 | 40.114 | 7.725 | −87.720 | 1.00 | 13.48 | C |
| ATOM | 211 | C | ALA | | 31 | 39.302 | 6.658 | −85.628 | 1.00 | 24.88 | C |
| ATOM | 212 | O | ALA | | 31 | 40.042 | 6.731 | −84.637 | 1.00 | 22.74 | O |
| ATOM | 213 | N | SER | | 32 | 38.609 | 5.568 | −85.931 | 1.00 | 20.13 | N |
| ATOM | 214 | CA | SER | | 32 | 38.743 | 4.357 | −85.134 | 1.00 | 21.45 | C |
| ATOM | 215 | CB | SER | | 32 | 37.705 | 4.328 | −84.009 | 1.00 | 14.79 | C |
| ATOM | 216 | OG | SER | | 32 | 36.415 | 4.637 | −84.488 | 1.00 | 20.62 | O |
| ATOM | 217 | C | SER | | 32 | 38.608 | 3.156 | −86.055 | 1.00 | 22.39 | C |
| ATOM | 218 | O | SER | | 32 | 37.779 | 3.144 | −86.963 | 1.00 | 21.77 | O |
| ATOM | 219 | N | GLN | | 33 | 39.448 | 2.152 | −85.837 | 1.00 | 25.22 | N |
| ATOM | 220 | CA | GLN | | 33 | 39.432 | 0.965 | −86.671 | 1.00 | 26.60 | C |
| ATOM | 221 | CB | GLN | | 33 | 40.275 | 1.212 | −87.925 | 1.00 | 21.68 | C |
| ATOM | 222 | CG | GLN | | 33 | 41.732 | 1.547 | −87.624 | 1.00 | 24.94 | C |
| ATOM | 223 | CD | GLN | | 33 | 42.468 | 2.115 | −88.826 | 1.00 | 29.02 | C |
| ATOM | 224 | OE1 | GLN | | 33 | 43.675 | 1.918 | −88.985 | 1.00 | 33.19 | O |
| ATOM | 225 | NE2 | GLN | | 33 | 41.746 | 2.842 | −89.670 | 1.00 | 25.93 | N |
| ATOM | 226 | C | GLN | | 33 | 39.991 | −0.221 | −85.901 | 1.00 | 27.17 | C |
| ATOM | 227 | O | GLN | | 33 | 40.700 | −0.048 | −84.907 | 1.00 | 25.99 | O |
| ATOM | 228 | N | TRP | | 34 | 39.682 | −1.424 | −86.373 | 1.00 | 26.47 | N |
| ATOM | 229 | CA | TRP | | 34 | 40.162 | −2.640 | −85.731 | 1.00 | 26.93 | C |
| ATOM | 230 | CB | TRP | | 34 | 39.362 | −3.854 | −86.216 | 1.00 | 23.74 | C |
| ATOM | 231 | CG | TRP | | 34 | 37.924 | −3.811 | −85.832 | 1.00 | 21.68 | C |
| ATOM | 232 | CD2 | TRP | | 34 | 37.391 | −3.839 | −84.507 | 1.00 | 22.32 | C |
| ATOM | 233 | CE2 | TRP | | 34 | 35.986 | −3.796 | −84.622 | 1.00 | 22.12 | C |
| ATOM | 234 | CE3 | TRP | | 34 | 37.964 | −3.897 | −83.234 | 1.00 | 22.16 | C |
| ATOM | 235 | CD1 | TRP | | 34 | 36.856 | −3.750 | −86.673 | 1.00 | 21.45 | C |
| ATOM | 236 | NE1 | TRP | | 34 | 35.687 | −3.742 | −85.957 | 1.00 | 20.94 | N |
| ATOM | 237 | CZ2 | TRP | | 34 | 35.144 | −3.810 | −83.513 | 1.00 | 23.32 | C |
| ATOM | 238 | CZ3 | TRP | | 34 | 37.130 | −3.910 | −82.134 | 1.00 | 23.35 | C |
| ATOM | 239 | CH2 | TRP | | 34 | 35.732 | −3.867 | −82.281 | 1.00 | 23.25 | C |
| ATOM | 240 | C | TRP | | 34 | 41.628 | −2.872 | −86.024 | 1.00 | 28.82 | C |
| ATOM | 241 | O | TRP | | 34 | 42.383 | −3.307 | −85.149 | 1.00 | 27.88 | O |
| ATOM | 242 | N | GLU | | 35 | 42.027 | −2.590 | −87.259 | 1.00 | 22.19 | N |
| ATOM | 243 | CA | GLU | | 35 | 43.413 | −2.790 | −87.683 | 1.00 | 25.69 | C |
| ATOM | 244 | CB | GLU | | 35 | 43.511 | −2.799 | −89.210 | 1.00 | 113.10 | C |
| ATOM | 245 | CG | GLU | | 35 | 43.358 | −4.168 | −89.844 | 1.00 | 114.00 | C |
| ATOM | 246 | CD | GLU | | 35 | 44.025 | −4.245 | −91.206 | 1.00 | 114.83 | C |
| ATOM | 247 | OE1 | GLU | | 35 | 44.009 | −5.331 | −91.819 | 1.00 | 114.85 | O |
| ATOM | 248 | OE2 | GLU | | 35 | 44.573 | −3.220 | −91.663 | 1.00 | 113.87 | O |
| ATOM | 249 | C | GLU | | 35 | 44.411 | −1.776 | −87.142 | 1.00 | 29.41 | C |
| ATOM | 250 | O | GLU | | 35 | 44.112 | −0.584 | −87.014 | 1.00 | 28.07 | O |
| ATOM | 251 | N | ARG | | 36 | 45.609 | −2.260 | −86.838 | 1.00 | 41.41 | N |
| ATOM | 252 | CA | ARG | | 36 | 46.657 | −1.394 | −86.332 | 1.00 | 47.16 | C |
| ATOM | 253 | CB | ARG | | 36 | 47.825 | −2.221 | −85.791 | 1.00 | 67.63 | C |
| ATOM | 254 | CG | ARG | | 36 | 48.898 | −1.381 | −85.132 | 1.00 | 72.03 | C |
| ATOM | 255 | CD | ARG | | 36 | 49.729 | −2.193 | −84.152 | 1.00 | 75.16 | C |
| ATOM | 256 | NE | ARG | | 36 | 50.751 | −1.372 | −83.507 | 1.00 | 77.90 | N |
| ATOM | 257 | CZ | ARG | | 36 | 51.475 | −1.758 | −82.462 | 1.00 | 79.60 | C |
| ATOM | 258 | NH1 | ARG | | 36 | 51.291 | −2.960 | −81.933 | 1.00 | 80.36 | N |
| ATOM | 259 | NH2 | ARG | | 36 | 52.385 | −0.941 | −81.946 | 1.00 | 80.00 | N |
| ATOM | 260 | C | ARG | | 36 | 47.105 | −0.521 | −87.493 | 1.00 | 49.44 | C |
| ATOM | 261 | O | ARG | | 36 | 47.498 | −1.024 | −88.547 | 1.00 | 49.61 | O |
| ATOM | 262 | N | PRO | | 37 | 47.034 | 0.806 | −87.315 | 1.00 | 38.51 | N |
| ATOM | 263 | CD | PRO | | 37 | 46.684 | 1.454 | −86.038 | 1.00 | 41.78 | C |
| ATOM | 264 | CA | PRO | | 37 | 47.412 | 1.806 | −88.317 | 1.00 | 41.36 | C |
| ATOM | 265 | CB | PRO | | 37 | 47.091 | 3.125 | −87.618 | 1.00 | 43.25 | C |
| ATOM | 266 | CG | PRO | | 37 | 47.346 | 2.804 | −86.175 | 1.00 | 42.40 | C |
| ATOM | 267 | C | PRO | | 37 | 48.864 | 1.733 | −88.778 | 1.00 | 44.20 | C |
| ATOM | 268 | O | PRO | | 37 | 49.771 | 1.508 | −87.976 | 1.00 | 44.35 | O |
| ATOM | 269 | N | SER | | 38 | 49.066 | 1.932 | −90.078 | 1.00 | 87.02 | N |
| ATOM | 270 | CA | SER | | 38 | 50.397 | 1.909 | −90.676 | 1.00 | 89.01 | C |
| ATOM | 271 | CB | SER | | 38 | 51.164 | 3.173 | −90.280 | 1.00 | 52.07 | C |
| ATOM | 272 | OG | SER | | 38 | 50.428 | 4.332 | −90.629 | 1.00 | 50.09 | O |
| ATOM | 273 | C | SER | | 38 | 51.189 | 0.675 | −90.259 | 1.00 | 90.20 | C |
| ATOM | 274 | O | SER | | 38 | 52.230 | 0.370 | −90.840 | 1.00 | 91.79 | O |
| ATOM | 275 | N | GLU | | 51 | 54.661 | 14.646 | −92.609 | 1.00 | 92.08 | N |
| ATOM | 276 | CA | GLU | | 51 | 54.083 | 15.985 | −92.549 | 1.00 | 91.73 | C |
| ATOM | 277 | CB | GLU | | 51 | 54.678 | 16.760 | −91.371 | 1.00 | 69.83 | C |
| ATOM | 278 | CG | GLU | | 51 | 54.217 | 18.214 | −91.243 | 1.00 | 68.90 | C |
| ATOM | 279 | CD | GLU | | 51 | 52.766 | 18.356 | −90.819 | 1.00 | 68.63 | C |
| ATOM | 280 | OE1 | GLU | | 51 | 52.358 | 17.695 | −89.841 | 1.00 | 67.72 | O |
| ATOM | 281 | OE2 | GLU | | 51 | 52.037 | 19.144 | −91.456 | 1.00 | 69.14 | O |
| ATOM | 282 | C | GLU | | 51 | 54.343 | 16.739 | −93.847 | 1.00 | 91.20 | C |
| ATOM | 283 | O | GLU | | 51 | 55.210 | 17.611 | −93.912 | 1.00 | 91.59 | O |
| ATOM | 284 | N | PRO | | 52 | 53.594 | 16.400 | −94.907 | 1.00 | 80.47 | N |
| ATOM | 285 | CD | PRO | | 52 | 52.601 | 15.314 | −94.967 | 1.00 | 63.31 | C |
| ATOM | 286 | CA | PRO | | 52 | 53.741 | 17.048 | −96.213 | 1.00 | 78.63 | C |
| ATOM | 287 | CB | PRO | | 52 | 52.656 | 16.380 | −97.052 | 1.00 | 62.58 | C |
| ATOM | 288 | CG | PRO | | 52 | 52.557 | 15.009 | −96.443 | 1.00 | 63.23 | C |
| ATOM | 289 | C | PRO | | 52 | 53.532 | 18.553 | −96.101 | 1.00 | 76.98 | C |
| ATOM | 290 | O | PRO | | 52 | 52.906 | 19.031 | −95.159 | 1.00 | 77.88 | O |
| ATOM | 291 | N | ALA | | 53 | 54.062 | 19.295 | −97.064 | 1.00 | 49.73 | N |
| ATOM | 292 | CA | ALA | | 53 | 53.916 | 20.742 | −97.069 | 1.00 | 47.22 | C |
| ATOM | 293 | CB | ALA | | 53 | 54.698 | 21.340 | −98.224 | 1.00 | 50.21 | C |
| ATOM | 294 | C | ALA | | 53 | 52.439 | 21.069 | −97.214 | 1.00 | 45.17 | C |
| ATOM | 295 | O | ALA | | 53 | 51.914 | 21.969 | −96.552 | 1.00 | 44.10 | O |
| ATOM | 296 | N | ARG | | 54 | 51.769 | 20.322 | −98.086 | 1.00 | 45.37 | N |
| ATOM | 297 | CA | ARG | | 54 | 50.346 | 20.527 | −98.329 | 1.00 | 42.09 | C |
| ATOM | 298 | CB | ARG | | 54 | 50.145 | 21.379 | −99.583 | 1.00 | 54.96 | C |
| ATOM | 299 | CG | ARG | | 54 | 51.071 | 22.574 | −99.703 | 1.00 | 59.03 | C |
| ATOM | 300 | CD | ARG | | 54 | 50.896 | 23.234 | −101.060 | 1.00 | 62.09 | C |
| ATOM | 301 | NE | ARG | | 54 | 49.611 | 23.917 | −101.168 | 1.00 | 63.23 | N |
| ATOM | 302 | CZ | ARG | | 54 | 48.992 | 24.166 | −102.318 | 1.00 | 65.00 | C |
| ATOM | 303 | NH1 | ARG | | 54 | 49.537 | 23.781 | −103.464 | 1.00 | 65.68 | N |
| ATOM | 304 | NH2 | ARG | | 54 | 47.834 | 24.814 | −102.324 | 1.00 | 65.50 | N |
| ATOM | 305 | C | ARG | | 54 | 49.621 | 19.199 | −98.527 | 1.00 | 38.23 | C |
| ATOM | 306 | O | ARG | | 54 | 50.241 | 18.165 | −98.784 | 1.00 | 37.73 | O |
| ATOM | 307 | N | VAL | | 55 | 48.302 | 19.236 | −98.396 | 1.00 | 32.71 | N |
| ATOM | 308 | CA | VAL | | 55 | 47.482 | 18.052 | −98.611 | 1.00 | 28.68 | C |
| ATOM | 309 | CB | VAL | | 55 | 47.144 | 17.298 | −97.293 | 1.00 | 27.56 | C |
| ATOM | 310 | CG1 | VAL | | 55 | 48.423 | 16.893 | −96.572 | 1.00 | 25.19 | C |
| ATOM | 311 | CG2 | VAL | | 55 | 46.260 | 18.161 | −96.401 | 1.00 | 24.60 | C |
| ATOM | 312 | C | VAL | | 55 | 46.190 | 18.530 | −99.243 | 1.00 | 27.59 | C |
| ATOM | 313 | O | VAL | | 55 | 45.860 | 19.718 | −99.188 | 1.00 | 26.57 | O |
| ATOM | 314 | N | ARG | | 56 | 45.467 | 17.606 | −99.860 | 1.00 | 28.91 | N |
| ATOM | 315 | CA | ARG | | 56 | 44.200 | 17.937 | −100.490 | 1.00 | 27.57 | C |
| ATOM | 316 | CB | ARG | | 56 | 44.271 | 17.629 | −101.985 | 1.00 | 25.81 | C |
| ATOM | 317 | CG | ARG | | 56 | 43.002 | 17.945 | −102.733 | 1.00 | 26.97 | C |
| ATOM | 318 | CD | ARG | | 56 | 43.102 | 17.516 | −104.196 | 1.00 | 28.35 | C |
| ATOM | 319 | NE | ARG | | 56 | 41.908 | 17.897 | −104.944 | 1.00 | 28.83 | N |
| ATOM | 320 | CZ | ARG | | 56 | 41.713 | 17.638 | −106.234 | 1.00 | 30.14 | C |
| ATOM | 321 | NH1 | ARG | | 56 | 42.639 | 16.993 | −106.932 | 1.00 | 28.54 | N |
| ATOM | 322 | NH2 | ARG | | 56 | 40.590 | 18.029 | −106.824 | 1.00 | 29.45 | N |
| ATOM | 323 | C | ARG | | 56 | 43.106 | 17.100 | −99.831 | 1.00 | 26.22 | C |
| ATOM | 324 | O | ARG | | 56 | 43.262 | 15.892 | −99.664 | 1.00 | 26.54 | O |
| ATOM | 325 | N | CYS | | 57 | 42.002 | 17.739 | −99.457 | 1.00 | 27.61 | N |
| ATOM | 326 | CA | CYS | | 57 | 40.911 | 17.021 | −98.815 | 1.00 | 27.07 | C |
| ATOM | 327 | CB | CYS | | 57 | 40.937 | 17.251 | −97.295 | 1.00 | 24.51 | C |
| ATOM | 328 | SG | CYS | | 57 | 42.456 | 16.771 | −96.448 | 1.00 | 23.28 | S |
| ATOM | 329 | C | CYS | | 57 | 39.534 | 17.419 | −99.313 | 1.00 | 27.66 | C |
| ATOM | 330 | O | CYS | | 57 | 39.341 | 18.492 | −99.901 | 1.00 | 27.75 | O |
| ATOM | 331 | N | SER | | 58 | 38.575 | 16.528 | −99.093 | 1.00 | 18.97 | N |
| ATOM | 332 | CA | SER | | 58 | 37.188 | 16.815 | −99.408 | 1.00 | 18.86 | C |
| ATOM | 333 | CB | SER | | 58 | 36.589 | 15.788 | −100.367 | 1.00 | 18.57 | C |
| ATOM | 334 | OG | SER | | 58 | 37.161 | 15.901 | −101.653 | 1.00 | 17.65 | O |
| ATOM | 335 | C | SER | | 58 | 36.543 | 16.691 | −98.033 | 1.00 | 19.26 | C |
| ATOM | 336 | O | SER | | 58 | 37.154 | 16.148 | −97.110 | 1.00 | 19.18 | O |
| ATOM | 337 | N | HIS | | 59 | 35.340 | 17.213 | −97.863 | 1.00 | 19.54 | N |
| ATOM | 338 | CA | HIS | | 59 | 34.699 | 17.082 | −96.572 | 1.00 | 19.45 | C |
| ATOM | 339 | CB | HIS | | 59 | 35.213 | 18.150 | −95.587 | 1.00 | 22.98 | C |
| ATOM | 340 | CG | HIS | | 59 | 34.482 | 19.457 | −95.650 | 1.00 | 25.80 | C |
| ATOM | 341 | CD2 | HIS | | 59 | 33.925 | 20.209 | −94.671 | 1.00 | 24.13 | C |
| ATOM | 342 | ND1 | HIS | | 59 | 34.288 | 20.154 | −96.824 | 1.00 | 25.49 | N |
| ATOM | 343 | CE1 | HIS | | 59 | 33.644 | 21.280 | −96.566 | 1.00 | 23.15 | C |
| ATOM | 344 | NE2 | HIS | | 59 | 33.413 | 21.338 | −95.266 | 1.00 | 27.95 | N |
| ATOM | 345 | C | HIS | | 59 | 33.205 | 17.177 | −96.728 | 1.00 | 19.68 | C |
| ATOM | 346 | O | HIS | | 59 | 32.694 | 17.606 | −97.772 | 1.00 | 18.30 | O |
| ATOM | 347 | N | LEU | | 60 | 32.506 | 16.726 | −95.701 | 1.00 | 27.73 | N |
| ATOM | 348 | CA | LEU | | 60 | 31.060 | 16.777 | −95.683 | 1.00 | 28.86 | C |
| ATOM | 349 | CB | LEU | | 60 | 30.466 | 15.373 | −95.663 | 1.00 | 17.06 | C |
| ATOM | 350 | CG | LEU | | 60 | 28.944 | 15.258 | −95.812 | 1.00 | 16.21 | C |
| ATOM | 351 | CD1 | LEU | | 60 | 28.592 | 13.791 | −96.102 | 1.00 | 16.50 | C |
| ATOM | 352 | CD2 | LEU | | 60 | 28.239 | 15.749 | −94.552 | 1.00 | 15.55 | C |
| ATOM | 353 | C | LEU | | 60 | 30.781 | 17.494 | −94.379 | 1.00 | 29.80 | C |
| ATOM | 354 | O | LEU | | 60 | 31.179 | 17.037 | −93.303 | 1.00 | 28.50 | O |
| ATOM | 355 | N | LEU | | 61 | 30.108 | 18.631 | −94.481 | 1.00 | 23.42 | N |
| ATOM | 356 | CA | LEU | | 61 | 29.814 | 19.430 | −93.309 | 1.00 | 24.50 | C |
| ATOM | 357 | CB | LEU | | 61 | 30.235 | 20.883 | −93.569 | 1.00 | 20.98 | C |
| ATOM | 358 | CG | LEU | | 61 | 29.742 | 21.968 | −92.603 | 1.00 | 21.15 | C |
| ATOM | 359 | CD1 | LEU | | 61 | 30.259 | 21.704 | −91.188 | 1.00 | 23.45 | C |
| ATOM | 360 | CD2 | LEU | | 61 | 30.215 | 23.332 | −93.110 | 1.00 | 23.24 | C |
| ATOM | 361 | C | LEU | | 61 | 28.351 | 19.387 | −92.917 | 1.00 | 24.25 | C |
| ATOM | 362 | O | LEU | | 61 | 27.468 | 19.458 | −93.767 | 1.00 | 22.30 | O |
| ATOM | 363 | N | VAL | | 62 | 28.100 | 19.241 | −91.622 | 1.00 | 23.07 | N |
| ATOM | 364 | CA | VAL | | 62 | 26.733 | 19.260 | −91.126 | 1.00 | 23.20 | C |
| ATOM | 365 | CB | VAL | | 62 | 26.315 | 17.943 | −90.474 | 1.00 | 22.43 | C |
| ATOM | 366 | CG1 | VAL | | 62 | 24.891 | 18.079 | −89.908 | 1.00 | 22.75 | C |
| ATOM | 367 | CG2 | VAL | | 62 | 26.372 | 16.820 | −91.504 | 1.00 | 23.21 | C |
| ATOM | 368 | C | VAL | | 62 | 26.729 | 20.355 | −90.080 | 1.00 | 24.20 | C |
| ATOM | 369 | O | VAL | | 62 | 27.423 | 20.259 | −89.052 | 1.00 | 21.83 | O |
| ATOM | 370 | N | LYS | | 63 | 25.971 | 21.409 | −90.358 | 1.00 | 31.76 | N |
| ATOM | 371 | CA | LYS | | 63 | 25.886 | 22.547 | −89.456 | 1.00 | 34.19 | C |
| ATOM | 372 | CB | LYS | | 63 | 25.557 | 23.817 | −90.233 | 1.00 | 37.41 | C |
| ATOM | 373 | CG | LYS | | 63 | 26.669 | 24.304 | −91.119 | 1.00 | 39.47 | C |
| ATOM | 374 | CD | LYS | | 63 | 26.309 | 25.663 | −91.666 | 1.00 | 40.11 | C |
| ATOM | 375 | CE | LYS | | 63 | 27.374 | 26.178 | −92.593 | 1.00 | 38.76 | C |
| ATOM | 376 | NZ | LYS | | 63 | 27.057 | 27.566 | −93.025 | 1.00 | 39.14 | N |
| ATOM | 377 | C | LYS | | 63 | 24.826 | 22.355 | −88.400 | 1.00 | 34.22 | C |
| ATOM | 378 | O | LYS | | 63 | 24.014 | 21.436 | −88.482 | 1.00 | 33.49 | O |
| ATOM | 379 | N | HIS | | 64 | 24.843 | 23.245 | −87.416 | 1.00 | 38.80 | N |
| ATOM | 380 | CA | HIS | | 64 | 23.875 | 23.227 | −86.336 | 1.00 | 39.34 | C |
| ATOM | 381 | CB | HIS | | 64 | 24.374 | 22.350 | −85.186 | 1.00 | 26.15 | C |
| ATOM | 382 | CG | HIS | | 64 | 25.764 | 22.665 | −84.739 | 1.00 | 27.92 | C |
| ATOM | 383 | CD2 | HIS | | 64 | 26.911 | 21.950 | −84.822 | 1.00 | 28.71 | C |
| ATOM | 384 | ND1 | HIS | | 64 | 26.092 | 23.843 | −84.104 | 1.00 | 27.69 | N |
| ATOM | 385 | CE1 | HIS | | 64 | 27.381 | 23.840 | −83.815 | 1.00 | 30.59 | C |
| ATOM | 386 | NE2 | HIS | | 64 | 27.902 | 22.702 | −84.240 | 1.00 | 29.36 | N |
| ATOM | 387 | C | HIS | | 64 | 23.609 | 24.657 | −85.866 | 1.00 | 39.50 | C |
| ATOM | 388 | O | HIS | | 64 | 24.286 | 25.596 | −86.298 | 1.00 | 38.46 | O |
| ATOM | 389 | N | SER | | 65 | 22.620 | 24.811 | −84.991 | 1.00 | 38.20 | N |
| ATOM | 390 | CA | SER | | 65 | 22.227 | 26.120 | −84.482 | 1.00 | 38.29 | C |
| ATOM | 391 | CB | SER | | 65 | 21.107 | 25.965 | −83.446 | 1.00 | 31.03 | C |
| ATOM | 392 | OG | SER | | 65 | 21.538 | 25.220 | −82.321 | 1.00 | 32.58 | O |
| ATOM | 393 | C | SER | | 65 | 23.365 | 26.960 | −83.901 | 1.00 | 39.21 | C |
| ATOM | 394 | O | SER | | 65 | 23.274 | 28.183 | −83.879 | 1.00 | 38.51 | O |
| ATOM | 395 | N | GLN | | 66 | 24.432 | 26.321 | −83.432 | 1.00 | 33.43 | N |
| ATOM | 396 | CA | GLN | | 66 | 25.552 | 27.082 | −82.881 | 1.00 | 34.22 | C |
| ATOM | 397 | CB | GLN | | 66 | 26.079 | 26.434 | −81.588 | 1.00 | 34.35 | C |
| ATOM | 398 | CG | GLN | | 66 | 25.156 | 26.604 | −80.370 | 1.00 | 34.76 | C |
| ATOM | 399 | CD | GLN | | 66 | 25.801 | 26.146 | −79.056 | 1.00 | 38.15 | C |
| ATOM | 400 | OE1 | GLN | | 66 | 26.820 | 26.693 | −78.624 | 1.00 | 35.78 | O |
| ATOM | 401 | NE2 | GLN | | 66 | 25.209 | 25.136 | −78.422 | 1.00 | 37.60 | N |
| ATOM | 402 | C | GLN | | 66 | 26.707 | 27.293 | −83.859 | 1.00 | 33.93 | C |
| ATOM | 403 | O | GLN | | 66 | 27.775 | 27.762 | −83.459 | 1.00 | 36.71 | O |
| ATOM | 404 | N | SER | | 67 | 26.506 | 26.949 | −85.132 | 1.00 | 24.38 | N |
| ATOM | 405 | CA | SER | | 67 | 27.549 | 27.139 | −86.146 | 1.00 | 25.76 | C |
| ATOM | 406 | CB | SER | | 67 | 27.135 | 26.483 | −87.477 | 1.00 | 34.89 | C |
| ATOM | 407 | OG | SER | | 67 | 27.106 | 25.065 | −87.383 | 1.00 | 31.75 | O |
| ATOM | 408 | C | SER | | 67 | 27.776 | 28.641 | −86.361 | 1.00 | 27.88 | C |
| ATOM | 409 | O | SER | | 67 | 26.818 | 29.419 | −86.334 | 1.00 | 27.78 | O |
| ATOM | 410 | N | ARG | | 68 | 29.028 | 29.053 | −86.582 | 1.00 | 35.49 | N |
| ATOM | 411 | CA | ARG | | 68 | 29.313 | 30.480 | −86.781 | 1.00 | 39.98 | C |
| ATOM | 412 | CB | ARG | | 68 | 30.746 | 30.720 | −87.289 | 1.00 | 92.68 | C |
| ATOM | 413 | CG | ARG | | 68 | 31.456 | 29.514 | −87.873 | 1.00 | 99.66 | C |
| ATOM | 414 | CD | ARG | | 68 | 32.616 | 29.917 | −88.797 | 1.00 | 104.74 | C |
| ATOM | 415 | NE | ARG | | 68 | 33.740 | 30.557 | −88.124 | 1.00 | 108.46 | N |
| ATOM | 416 | CZ | ARG | | 68 | 34.781 | 31.080 | −88.764 | 1.00 | 110.64 | C |
| ATOM | 417 | NH1 | ARG | | 68 | 34.832 | 31.043 | −90.088 | 1.00 | 110.85 | N |
| ATOM | 418 | NH2 | ARG | | 68 | 35.780 | 31.623 | −88.081 | 1.00 | 112.10 | N |
| ATOM | 419 | C | ARG | | 68 | 28.313 | 31.130 | −87.736 | 1.00 | 39.81 | C |
| ATOM | 420 | O | ARG | | 68 | 28.006 | 32.312 | −87.607 | 1.00 | 39.99 | O |
| ATOM | 421 | N | ARG | | 69 | 27.797 | 30.351 | −88.681 | 1.00 | 46.18 | N |
| ATOM | 422 | CA | ARG | | 69 | 26.813 | 30.850 | −89.635 | 1.00 | 44.85 | C |
| ATOM | 423 | CB | ARG | | 69 | 27.493 | 31.223 | −90.950 | 1.00 | 59.80 | C |
| ATOM | 424 | CG | ARG | | 69 | 28.495 | 32.359 | −90.826 | 1.00 | 63.97 | C |
| ATOM | 425 | CD | ARG | | 69 | 29.354 | 32.430 | −92.064 | 1.00 | 68.66 | C |
| ATOM | 426 | NE | ARG | | 69 | 28.542 | 32.337 | −93.272 | 1.00 | 72.47 | N |
| ATOM | 427 | CZ | ARG | | 69 | 29.037 | 32.173 | −94.494 | 1.00 | 75.20 | C |
| ATOM | 428 | NH1 | ARG | | 69 | 30.349 | 32.083 | −94.672 | 1.00 | 76.93 | N |
| ATOM | 429 | NH2 | ARG | | 69 | 28.222 | 32.096 | −95.538 | 1.00 | 76.34 | N |
| ATOM | 430 | C | ARG | | 69 | 25.768 | 29.765 | −89.877 | 1.00 | 43.28 | C |
| ATOM | 431 | O | ARG | | 69 | 25.914 | 28.934 | −90.772 | 1.00 | 41.76 | O |
| ATOM | 432 | N | PRO | | 70 | 24.691 | 29.771 | −89.078 | 1.00 | 30.50 | N |
| ATOM | 433 | CD | PRO | | 70 | 24.448 | 30.802 | −88.052 | 1.00 | 37.86 | C |
| ATOM | 434 | CA | PRO | | 70 | 23.577 | 28.816 | −89.134 | 1.00 | 29.58 | C |
| ATOM | 435 | CB | PRO | | 70 | 22.765 | 29.170 | −87.889 | 1.00 | 36.97 | C |
| ATOM | 436 | CG | PRO | | 70 | 22.968 | 30.647 | −87.775 | 1.00 | 37.19 | C |
| ATOM | 437 | C | PRO | | 70 | 22.739 | 28.847 | −90.408 | 1.00 | 28.58 | C |
| ATOM | 438 | O | PRO | | 70 | 21.505 | 28.850 | −90.350 | 1.00 | 27.99 | O |
| ATOM | 439 | N | SER | | 71 | 23.413 | 28.846 | −91.555 | 1.00 | 31.28 | N |
| ATOM | 440 | CA | SER | | 71 | 22.732 | 28.875 | −92.847 | 1.00 | 28.94 | C |
| ATOM | 441 | CB | SER | | 71 | 22.449 | 30.324 | −93.252 | 1.00 | 58.35 | C |
| ATOM | 442 | OG | SER | | 71 | 21.771 | 30.391 | −94.494 | 1.00 | 63.72 | O |
| ATOM | 443 | C | SER | | 71 | 23.589 | 28.206 | −93.922 | 1.00 | 26.93 | C |
| ATOM | 444 | O | SER | | 71 | 24.817 | 28.186 | −93.815 | 1.00 | 25.77 | O |
| ATOM | 445 | N | SER | | 72 | 22.945 | 27.657 | −94.950 | 1.00 | 27.32 | N |
| ATOM | 446 | CA | SER | | 72 | 23.669 | 27.004 | −96.043 | 1.00 | 27.77 | C |
| ATOM | 447 | CB | SER | | 72 | 24.090 | 25.588 | −95.639 | 1.00 | 32.27 | C |
| ATOM | 448 | OG | SER | | 72 | 22.970 | 24.718 | −95.683 | 1.00 | 28.53 | O |
| ATOM | 449 | C | SER | | 72 | 22.784 | 26.908 | −97.277 | 1.00 | 27.26 | C |
| ATOM | 450 | O | SER | | 72 | 21.598 | 27.208 | −97.211 | 1.00 | 27.46 | O |
| ATOM | 451 | N | TRP | | 73 | 23.357 | 26.466 | −98.393 | 1.00 | 28.76 | N |
| ATOM | 452 | CA | TRP | | 73 | 22.596 | 26.318 | −99.631 | 1.00 | 27.44 | C |
| ATOM | 453 | CB | TRP | | 73 | 23.510 | 25.873 | −100.783 | 1.00 | 26.47 | C |
| ATOM | 454 | CG | TRP | | 73 | 24.047 | 24.460 | −100.637 | 1.00 | 27.29 | C |
| ATOM | 455 | CD2 | TRP | | 73 | 23.430 | 23.252 | −101.108 | 1.00 | 26.36 | C |
| ATOM | 456 | CE2 | TRP | | 73 | 24.267 | 22.180 | −100.730 | 1.00 | 25.53 | C |
| ATOM | 457 | CE3 | TRP | | 73 | 22.252 | 22.973 | −101.810 | 1.00 | 26.63 | C |
| ATOM | 458 | CD1 | TRP | | 73 | 25.199 | 24.079 | −100.010 | 1.00 | 26.10 | C |
| ATOM | 459 | NE1 | TRP | | 73 | 25.339 | 22.712 | −100.062 | 1.00 | 25.00 | N |
| ATOM | 460 | CZ2 | TRP | | 73 | 23.964 | 20.853 | −101.031 | 1.00 | 25.25 | C |
| ATOM | 461 | CZ3 | TRP | | 73 | 21.951 | 21.649 | −102.106 | 1.00 | 26.55 | C |
| ATOM | 462 | CH2 | TRP | | 73 | 22.805 | 20.607 | −101.715 | 1.00 | 25.37 | C |
| ATOM | 463 | C | TRP | | 73 | 21.461 | 25.304 | −99.472 | 1.00 | 27.18 | C |
| ATOM | 464 | O | TRP | | 73 | 20.519 | 25.297 | −100.261 | 1.00 | 27.30 | O |
| ATOM | 465 | N | ARG | | 74 | 21.547 | 24.448 | −98.456 | 1.00 | 30.87 | N |
| ATOM | 466 | CA | ARG | | 74 | 20.508 | 23.442 | −98.232 | 1.00 | 30.38 | C |
| ATOM | 467 | CB | ARG | | 74 | 21.074 | 22.246 | −97.457 | 1.00 | 28.25 | C |
| ATOM | 468 | CG | ARG | | 74 | 22.278 | 21.581 | −98.127 | 1.00 | 27.61 | C |
| ATOM | 469 | CD | ARG | | 74 | 22.506 | 20.191 | −97.548 | 1.00 | 27.22 | C |
| ATOM | 470 | NE | ARG | | 74 | 21.346 | 19.345 | −97.807 | 1.00 | 27.38 | N |
| ATOM | 471 | CZ | ARG | | 74 | 20.740 | 18.605 | −96.887 | 1.00 | 28.74 | C |
| ATOM | 472 | NH1 | ARG | | 74 | 21.185 | 18.603 | −95.636 | 1.00 | 32.43 | N |
| ATOM | 473 | NH2 | ARG | | 74 | 19.685 | 17.874 | −97.221 | 1.00 | 30.48 | N |
| ATOM | 474 | C | ARG | | 74 | 19.286 | 23.998 | −97.492 | 1.00 | 31.33 | C |
| ATOM | 475 | O | ARG | | 74 | 18.160 | 23.578 | −97.742 | 1.00 | 30.12 | O |
| ATOM | 476 | N | GLN | | 75 | 19.507 | 24.925 | −96.569 | 1.00 | 32.46 | N |
| ATOM | 477 | CA | GLN | | 75 | 18.399 | 25.530 | −95.838 | 1.00 | 34.90 | C |
| ATOM | 478 | CB | GLN | | 75 | 17.905 | 24.611 | −94.718 | 1.00 | 46.12 | C |
| ATOM | 479 | CG | GLN | | 75 | 18.962 | 24.174 | −93.739 | 1.00 | 49.44 | C |
| ATOM | 480 | CD | GLN | | 75 | 18.394 | 23.318 | −92.618 | 1.00 | 51.76 | C |
| ATOM | 481 | OE1 | GLN | | 75 | 19.128 | 22.836 | −91.755 | 1.00 | 51.77 | O |
| ATOM | 482 | NE2 | GLN | | 75 | 17.079 | 23.126 | −92.627 | 1.00 | 51.35 | N |
| ATOM | 483 | C | GLN | | 75 | 18.827 | 26.880 | −95.281 | 1.00 | 33.48 | C |
| ATOM | 484 | O | GLN | | 75 | 19.858 | 26.997 | −94.606 | 1.00 | 32.57 | O |
| ATOM | 485 | N | GLU | | 76 | 18.021 | 27.895 | −95.583 | 1.00 | 31.46 | N |
| ATOM | 486 | CA | GLU | | 76 | 18.295 | 29.266 | −95.182 | 1.00 | 29.82 | C |
| ATOM | 487 | CB | GLU | | 76 | 17.127 | 30.171 | −95.598 | 1.00 | 34.27 | C |
| ATOM | 488 | CG | GLU | | 76 | 17.477 | 31.654 | −95.589 | 1.00 | 33.68 | C |
| ATOM | 489 | CD | GLU | | 76 | 16.467 | 32.511 | −96.342 | 1.00 | 33.92 | C |
| ATOM | 490 | OE1 | GLU | | 76 | 16.710 | 33.730 | −96.470 | 1.00 | 33.77 | O |
| ATOM | 491 | OE2 | GLU | | 76 | 15.438 | 31.972 | −96.803 | 1.00 | 30.89 | O |
| ATOM | 492 | C | GLU | | 76 | 18.586 | 29.425 | −93.694 | 1.00 | 30.64 | C |
| ATOM | 493 | O | GLU | | 76 | 19.451 | 30.212 | −93.305 | 1.00 | 29.95 | O |
| ATOM | 494 | N | LYS | | 77 | 17.873 | 28.671 | −92.867 | 1.00 | 40.14 | N |
| ATOM | 495 | CA | LYS | | 77 | 18.067 | 28.742 | −91.425 | 1.00 | 42.89 | C |
| ATOM | 496 | CB | LYS | | 77 | 16.858 | 29.415 | −90.770 | 1.00 | 54.20 | C |
| ATOM | 497 | CG | LYS | | 77 | 16.979 | 29.589 | −89.266 | 1.00 | 58.94 | C |
| ATOM | 498 | CD | LYS | | 77 | 18.161 | 30.485 | −88.906 | 1.00 | 61.26 | C |
| ATOM | 499 | CE | LYS | | 77 | 18.216 | 30.758 | −87.407 | 1.00 | 61.74 | C |
| ATOM | 500 | NZ | LYS | | 77 | 16.943 | 31.351 | −86.903 | 1.00 | 63.16 | N |
| ATOM | 501 | C | LYS | | 77 | 18.271 | 27.354 | −90.823 | 1.00 | 42.48 | C |
| ATOM | 502 | O | LYS | | 77 | 17.316 | 26.591 | −90.665 | 1.00 | 42.05 | O |
| ATOM | 503 | N | ILE | | 78 | 19.517 | 27.031 | −90.492 | 1.00 | 32.29 | N |
| ATOM | 504 | CA | ILE | | 78 | 19.843 | 25.738 | −89.893 | 1.00 | 32.05 | C |
| ATOM | 505 | CB | ILE | | 78 | 21.361 | 25.478 | −89.919 | 1.00 | 43.39 | C |
| ATOM | 506 | CG2 | ILE | | 78 | 21.648 | 24.075 | −89.406 | 1.00 | 42.19 | C |
| ATOM | 507 | CG1 | ILE | | 78 | 21.906 | 25.640 | −91.340 | 1.00 | 44.66 | C |
| ATOM | 508 | CD1 | ILE | | 78 | 21.671 | 24.445 | −92.229 | 1.00 | 44.62 | C |
| ATOM | 509 | C | ILE | | 78 | 19.404 | 25.787 | −88.429 | 1.00 | 31.81 | C |
| ATOM | 510 | O | ILE | | 78 | 19.882 | 26.629 | −87.671 | 1.00 | 32.03 | O |
| ATOM | 511 | N | THR | | 79 | 18.522 | 24.879 | −88.026 | 1.00 | 41.78 | N |
| ATOM | 512 | CA | THR | | 79 | 18.026 | 24.868 | −86.655 | 1.00 | 41.88 | C |
| ATOM | 513 | CB | THR | | 79 | 16.499 | 25.017 | −86.650 | 1.00 | 37.05 | C |
| ATOM | 514 | OG1 | THR | | 79 | 15.911 | 23.916 | −87.353 | 1.00 | 38.12 | O |
| ATOM | 515 | CG2 | THR | | 79 | 16.093 | 26.317 | −87.330 | 1.00 | 38.02 | C |
| ATOM | 516 | C | THR | | 79 | 18.391 | 23.635 | −85.817 | 1.00 | 41.44 | C |
| ATOM | 517 | O | THR | | 79 | 18.244 | 23.643 | −84.594 | 1.00 | 40.19 | O |
| ATOM | 518 | N | ARG | | 80 | 18.863 | 22.578 | −86.468 | 1.00 | 30.81 | N |
| ATOM | 519 | CA | ARG | | 80 | 19.226 | 21.356 | −85.758 | 1.00 | 28.34 | C |
| ATOM | 520 | CB | ARG | | 80 | 19.839 | 20.346 | −86.734 | 1.00 | 30.37 | C |
| ATOM | 521 | CG | ARG | | 80 | 21.189 | 20.798 | −87.278 | 1.00 | 31.24 | C |
| ATOM | 522 | CD | ARG | | 80 | 21.912 | 19.709 | −88.049 | 1.00 | 31.66 | C |
| ATOM | 523 | NE | ARG | | 80 | 21.703 | 19.835 | −89.488 | 1.00 | 35.73 | N |
| ATOM | 524 | CZ | ARG | | 80 | 20.633 | 19.374 | −90.117 | 1.00 | 36.07 | C |
| ATOM | 525 | NH1 | ARG | | 80 | 19.689 | 18.757 | −89.417 | 1.00 | 37.81 | N |
| ATOM | 526 | NH2 | ARG | | 80 | 20.510 | 19.525 | −91.435 | 1.00 | 34.27 | N |
| ATOM | 527 | C | ARG | | 80 | 20.224 | 21.633 | −84.629 | 1.00 | 26.44 | C |
| ATOM | 528 | O | ARG | | 80 | 21.080 | 22.520 | −84.739 | 1.00 | 26.14 | O |
| ATOM | 529 | N | THR | | 81 | 20.114 | 20.866 | −83.547 | 1.00 | 27.39 | N |
| ATOM | 530 | CA | THR | | 81 | 21.019 | 21.015 | −82.414 | 1.00 | 29.21 | C |
| ATOM | 531 | CB | THR | | 81 | 20.466 | 20.314 | −81.151 | 1.00 | 27.15 | C |
| ATOM | 532 | OG1 | THR | | 81 | 20.562 | 18.892 | −81.304 | 1.00 | 28.13 | O |
| ATOM | 533 | CG2 | THR | | 81 | 19.002 | 20.684 | −80.933 | 1.00 | 28.09 | C |
| ATOM | 534 | C | THR | | 81 | 22.353 | 20.362 | −82.772 | 1.00 | 29.67 | C |
| ATOM | 535 | O | THR | | 81 | 22.442 | 19.624 | −83.748 | 1.00 | 29.35 | O |
| ATOM | 536 | N | LYS | | 82 | 23.386 | 20.629 | −81.983 | 1.00 | 28.05 | N |
| ATOM | 537 | CA | LYS | | 82 | 24.688 | 20.025 | −82.229 | 1.00 | 27.86 | C |
| ATOM | 538 | CB | LYS | | 82 | 25.716 | 20.526 | −81.219 | 1.00 | 41.11 | C |
| ATOM | 539 | CG | LYS | | 82 | 25.852 | 22.029 | −81.170 | 1.00 | 46.40 | C |
| ATOM | 540 | CD | LYS | | 82 | 27.119 | 22.425 | −80.443 | 1.00 | 48.62 | C |
| ATOM | 541 | CE | LYS | | 82 | 27.205 | 21.764 | −79.083 | 1.00 | 51.16 | C |
| ATOM | 542 | NZ | LYS | | 82 | 28.509 | 22.068 | −78.445 | 1.00 | 51.86 | N |
| ATOM | 543 | C | LYS | | 82 | 24.578 | 18.505 | −82.110 | 1.00 | 27.47 | C |
| ATOM | 544 | O | LYS | | 82 | 25.238 | 17.761 | −82.837 | 1.00 | 23.66 | O |
| ATOM | 545 | N | GLU | | 83 | 23.750 | 18.050 | −81.175 | 1.00 | 25.01 | N |
| ATOM | 546 | CA | GLU | | 83 | 23.573 | 16.623 | −80.962 | 1.00 | 25.48 | C |
| ATOM | 547 | CB | GLU | | 83 | 22.700 | 16.373 | −79.727 | 1.00 | 34.29 | C |
| ATOM | 548 | CG | GLU | | 83 | 23.368 | 16.790 | −78.413 | 1.00 | 39.06 | C |
| ATOM | 549 | CD | GLU | | 83 | 23.555 | 18.306 | −78.270 | 1.00 | 41.07 | C |
| ATOM | 550 | OE1 | GLU | | 83 | 24.368 | 18.723 | −77.417 | 1.00 | 44.38 | O |
| ATOM | 551 | OE2 | GLU | | 83 | 22.889 | 19.082 | −78.992 | 1.00 | 40.83 | O |
| ATOM | 552 | C | GLU | | 83 | 22.957 | 15.963 | −82.189 | 1.00 | 23.05 | C |
| ATOM | 553 | O | GLU | | 83 | 23.410 | 14.907 | −82.625 | 1.00 | 21.19 | O |
| ATOM | 554 | N | GLU | | 84 | 21.925 | 16.583 | −82.749 | 1.00 | 32.44 | N |
| ATOM | 555 | CA | GLU | | 84 | 21.278 | 16.026 | −83.931 | 1.00 | 32.29 | C |
| ATOM | 556 | CB | GLU | | 84 | 20.041 | 16.847 | −84.297 | 1.00 | 29.92 | C |
| ATOM | 557 | CG | GLU | | 84 | 19.008 | 16.944 | −83.179 | 1.00 | 33.27 | C |
| ATOM | 558 | CD | GLU | | 84 | 17.803 | 17.784 | −83.573 | 1.00 | 34.77 | C |
| ATOM | 559 | OE1 | GLU | | 84 | 18.000 | 18.929 | −84.034 | 1.00 | 33.01 | O |
| ATOM | 560 | OE2 | GLU | | 84 | 16.662 | 17.303 | −83.421 | 1.00 | 37.17 | O |
| ATOM | 561 | C | GLU | | 84 | 22.262 | 16.009 | −85.108 | 1.00 | 32.11 | C |
| ATOM | 562 | O | GLU | | 84 | 22.257 | 15.084 | −85.927 | 1.00 | 31.49 | O |
| ATOM | 563 | N | ALA | | 85 | 23.109 | 17.033 | −85.175 | 1.00 | 23.50 | N |
| ATOM | 564 | CA | ALA | | 85 | 24.098 | 17.144 | −86.235 | 1.00 | 23.22 | C |
| ATOM | 565 | CB | ALA | | 85 | 24.796 | 18.512 | −86.166 | 1.00 | 20.42 | C |
| ATOM | 566 | C | ALA | | 85 | 25.130 | 16.026 | −86.123 | 1.00 | 23.16 | C |
| ATOM | 567 | O | ALA | | 85 | 25.553 | 15.450 | −87.131 | 1.00 | 21.09 | O |
| ATOM | 568 | N | LEU | | 86 | 25.539 | 15.730 | −84.893 | 1.00 | 23.72 | N |
| ATOM | 569 | CA | LEU | | 86 | 26.513 | 14.678 | −84.666 | 1.00 | 23.10 | C |
| ATOM | 570 | CB | LEU | | 86 | 26.934 | 14.647 | −83.190 | 1.00 | 21.81 | C |
| ATOM | 571 | CG | LEU | | 86 | 27.918 | 13.548 | −82.752 | 1.00 | 23.22 | C |
| ATOM | 572 | CD1 | LEU | | 86 | 29.108 | 13.493 | −83.704 | 1.00 | 22.43 | C |
| ATOM | 573 | CD2 | LEU | | 86 | 28.385 | 13.822 | −81.306 | 1.00 | 22.52 | C |
| ATOM | 574 | C | LEU | | 86 | 25.904 | 13.342 | −85.084 | 1.00 | 23.86 | C |
| ATOM | 575 | O | LEU | | 86 | 26.590 | 12.496 | −85.646 | 1.00 | 21.37 | O |
| ATOM | 576 | N | GLU | | 87 | 24.609 | 13.162 | −84.830 | 1.00 | 25.62 | N |
| ATOM | 577 | CA | GLU | | 87 | 23.941 | 11.917 | −85.204 | 1.00 | 28.23 | C |
| ATOM | 578 | CB | GLU | | 87 | 22.531 | 11.863 | −84.615 | 1.00 | 52.02 | C |
| ATOM | 579 | CG | GLU | | 87 | 22.509 | 11.251 | −83.233 | 1.00 | 57.63 | C |
| ATOM | 580 | CD | GLU | | 87 | 23.234 | 9.915 | −83.201 | 1.00 | 63.43 | C |
| ATOM | 581 | OE1 | GLU | | 87 | 22.812 | 8.992 | −83.932 | 1.00 | 63.83 | O |
| ATOM | 582 | OE2 | GLU | | 87 | 24.231 | 9.789 | −82.456 | 1.00 | 66.82 | O |
| ATOM | 583 | C | GLU | | 87 | 23.880 | 11.710 | −86.712 | 1.00 | 27.09 | C |
| ATOM | 584 | O | GLU | | 87 | 23.992 | 10.579 | −87.200 | 1.00 | 24.91 | O |
| ATOM | 585 | N | LEU | | 88 | 23.688 | 12.801 | −87.448 | 1.00 | 24.58 | N |
| ATOM | 586 | CA | LEU | | 88 | 23.632 | 12.744 | −88.900 | 1.00 | 23.33 | C |
| ATOM | 587 | CB | LEU | | 88 | 23.219 | 14.114 | −89.457 | 1.00 | 22.15 | C |
| ATOM | 588 | CG | LEU | | 88 | 21.721 | 14.435 | −89.292 | 1.00 | 25.36 | C |
| ATOM | 589 | CD1 | LEU | | 88 | 21.428 | 15.941 | −89.461 | 1.00 | 23.78 | C |
| ATOM | 590 | CD2 | LEU | | 88 | 20.950 | 13.611 | −90.315 | 1.00 | 23.79 | C |
| ATOM | 591 | C | LEU | | 88 | 25.013 | 12.346 | −89.404 | 1.00 | 21.25 | C |
| ATOM | 592 | O | LEU | | 88 | 25.149 | 11.495 | −90.290 | 1.00 | 21.56 | O |
| ATOM | 593 | N | ILE | | 89 | 26.035 | 12.964 | −88.820 | 1.00 | 20.76 | N |
| ATOM | 594 | CA | ILE | | 89 | 27.416 | 12.682 | −89.171 | 1.00 | 20.56 | C |
| ATOM | 595 | CB | ILE | | 89 | 28.375 | 13.559 | −88.337 | 1.00 | 16.51 | C |
| ATOM | 596 | CG2 | ILE | | 89 | 29.778 | 12.958 | −88.336 | 1.00 | 16.76 | C |
| ATOM | 597 | CG1 | ILE | | 89 | 28.405 | 14.988 | −88.903 | 1.00 | 15.05 | C |
| ATOM | 598 | CD1 | ILE | | 89 | 29.296 | 15.144 | −90.156 | 1.00 | 14.12 | C |
| ATOM | 599 | C | ILE | | 89 | 27.709 | 11.204 | −88.922 | 1.00 | 22.38 | C |
| ATOM | 600 | O | ILE | | 89 | 28.293 | 10.532 | −89.773 | 1.00 | 20.93 | O |
| ATOM | 601 | N | ASN | | 90 | 27.289 | 10.696 | −87.764 | 1.00 | 20.29 | N |
| ATOM | 602 | CA | ASN | | 90 | 27.514 | 9.294 | −87.433 | 1.00 | 21.57 | C |
| ATOM | 603 | CB | ASN | | 90 | 27.026 | 8.976 | −86.017 | 1.00 | 23.78 | C |
| ATOM | 604 | CG | ASN | | 90 | 27.918 | 9.587 | −84.945 | 1.00 | 24.81 | C |
| ATOM | 605 | OD1 | ASN | | 90 | 29.118 | 9.774 | −85.153 | 1.00 | 28.53 | O |
| ATOM | 606 | ND2 | ASN | | 90 | 27.339 | 9.882 | −83.791 | 1.00 | 28.96 | N |
| ATOM | 607 | C | ASN | | 90 | 26.840 | 8.369 | −88.436 | 1.00 | 21.58 | C |
| ATOM | 608 | O | ASN | | 90 | 27.416 | 7.356 | −88.829 | 1.00 | 21.84 | O |
| ATOM | 609 | N | GLY | | 91 | 25.629 | 8.723 | −88.859 | 1.00 | 21.28 | N |
| ATOM | 610 | CA | GLY | | 91 | 24.925 | 7.904 | −89.835 | 1.00 | 21.25 | C |
| ATOM | 611 | C | GLY | | 91 | 25.655 | 7.865 | −91.175 | 1.00 | 22.34 | C |
| ATOM | 612 | O | GLY | | 91 | 25.777 | 6.817 | −91.808 | 1.00 | 21.66 | O |
| ATOM | 613 | N | TYR | | 92 | 26.156 | 9.016 | −91.606 | 1.00 | 18.22 | N |
| ATOM | 614 | CA | TYR | | 92 | 26.871 | 9.115 | −92.878 | 1.00 | 17.74 | C |
| ATOM | 615 | CB | TYR | | 92 | 27.219 | 10.582 | −93.171 | 1.00 | 21.82 | C |
| ATOM | 616 | CG | TYR | | 92 | 26.007 | 11.461 | −93.408 | 1.00 | 22.95 | C |
| ATOM | 617 | CD1 | TYR | | 92 | 26.006 | 12.804 | −93.032 | 1.00 | 23.15 | C |
| ATOM | 618 | CE1 | TYR | | 92 | 24.900 | 13.614 | −93.263 | 1.00 | 23.87 | C |
| ATOM | 619 | CD2 | TYR | | 92 | 24.867 | 10.951 | −94.022 | 1.00 | 22.47 | C |
| ATOM | 620 | CE2 | TYR | | 92 | 23.756 | 11.749 | −94.256 | 1.00 | 24.76 | C |
| ATOM | 621 | CZ | TYR | | 92 | 23.782 | 13.081 | −93.875 | 1.00 | 23.95 | C |
| ATOM | 622 | OH | TYR | | 92 | 22.687 | 13.875 | −94.128 | 1.00 | 23.63 | O |
| ATOM | 623 | C | TYR | | 92 | 28.140 | 8.276 | −92.840 | 1.00 | 14.83 | C |
| ATOM | 624 | O | TYR | | 92 | 28.456 | 7.576 | −93.802 | 1.00 | 16.50 | O |
| ATOM | 625 | N | ILE | | 93 | 28.867 | 8.359 | −91.728 | 1.00 | 14.02 | N |
| ATOM | 626 | CA | ILE | | 93 | 30.094 | 7.595 | −91.574 | 1.00 | 17.07 | C |
| ATOM | 627 | CB | ILE | | 93 | 30.787 | 7.931 | −90.224 | 1.00 | 18.29 | C |
| ATOM | 628 | CG2 | ILE | | 93 | 31.878 | 6.913 | −89.907 | 1.00 | 18.42 | C |
| ATOM | 629 | CG1 | ILE | | 93 | 31.386 | 9.345 | −90.295 | 1.00 | 18.84 | C |
| ATOM | 630 | CD1 | ILE | | 93 | 32.022 | 9.803 | −88.996 | 1.00 | 18.79 | C |
| ATOM | 631 | C | ILE | | 93 | 29.752 | 6.101 | −91.654 | 1.00 | 16.85 | C |
| ATOM | 632 | O | ILE | | 93 | 30.430 | 5.333 | −92.345 | 1.00 | 15.46 | O |
| ATOM | 633 | N | GLN | | 94 | 28.680 | 5.696 | −90.981 | 1.00 | 18.85 | N |
| ATOM | 634 | CA | GLN | | 94 | 28.284 | 4.290 | −91.017 | 1.00 | 22.05 | C |
| ATOM | 635 | CB | GLN | | 94 | 27.023 | 4.043 | −90.194 | 1.00 | 31.01 | C |
| ATOM | 636 | CG | GLN | | 94 | 27.235 | 4.075 | −88.698 | 1.00 | 33.71 | C |
| ATOM | 637 | CD | GLN | | 94 | 26.065 | 3.462 | −87.954 | 1.00 | 38.87 | C |
| ATOM | 638 | OE1 | GLN | | 94 | 24.923 | 3.910 | −88.090 | 1.00 | 39.90 | O |
| ATOM | 639 | NE2 | GLN | | 94 | 26.342 | 2.424 | −87.167 | 1.00 | 39.16 | N |
| ATOM | 640 | C | GLN | | 94 | 28.040 | 3.815 | −92.441 | 1.00 | 21.88 | C |
| ATOM | 641 | O | GLN | | 94 | 28.522 | 2.746 | −92.835 | 1.00 | 22.96 | O |
| ATOM | 642 | N | LYS | | 95 | 27.302 | 4.608 | −93.213 | 1.00 | 20.75 | N |
| ATOM | 643 | CA | LYS | | 95 | 27.000 | 4.237 | −94.589 | 1.00 | 20.25 | C |
| ATOM | 644 | CB | LYS | | 95 | 25.913 | 5.147 | −95.161 | 1.00 | 36.57 | C |
| ATOM | 645 | CG | LYS | | 95 | 24.515 | 4.846 | −94.627 | 1.00 | 41.80 | C |
| ATOM | 646 | CD | LYS | | 95 | 23.452 | 5.506 | −95.493 | 1.00 | 45.97 | C |
| ATOM | 647 | CE | LYS | | 95 | 22.041 | 5.102 | −95.078 | 1.00 | 48.42 | C |
| ATOM | 648 | NZ | LYS | | 95 | 21.657 | 5.647 | −93.747 | 1.00 | 52.23 | N |
| ATOM | 649 | C | LYS | | 95 | 28.221 | 4.252 | −95.492 | 1.00 | 19.28 | C |
| ATOM | 650 | O | LYS | | 95 | 28.330 | 3.448 | −96.419 | 1.00 | 19.18 | O |
| ATOM | 651 | N | ILE | | 96 | 29.138 | 5.175 | −95.236 | 1.00 | 19.74 | N |
| ATOM | 652 | CA | ILE | | 96 | 30.353 | 5.254 | −96.032 | 1.00 | 19.01 | C |
| ATOM | 653 | CB | ILE | | 96 | 31.161 | 6.525 | −95.676 | 1.00 | 20.13 | C |
| ATOM | 654 | CG2 | ILE | | 96 | 32.566 | 6.431 | −96.240 | 1.00 | 18.08 | C |
| ATOM | 655 | CG1 | ILE | | 96 | 30.416 | 7.766 | −96.187 | 1.00 | 20.05 | C |
| ATOM | 656 | CD1 | ILE | | 96 | 31.124 | 9.089 | −95.876 | 1.00 | 20.90 | C |
| ATOM | 657 | C | ILE | | 96 | 31.212 | 4.019 | −95.749 | 1.00 | 17.74 | C |
| ATOM | 658 | O | ILE | | 96 | 31.706 | 3.353 | −96.669 | 1.00 | 19.21 | O |
| ATOM | 659 | N | LYS | | 97 | 31.369 | 3.711 | −94.468 | 1.00 | 18.05 | N |
| ATOM | 660 | CA | LYS | | 97 | 32.176 | 2.570 | −94.070 | 1.00 | 18.43 | C |
| ATOM | 661 | CB | LYS | | 97 | 32.412 | 2.573 | −92.559 | 1.00 | 23.17 | C |
| ATOM | 662 | CG | LYS | | 97 | 33.542 | 3.466 | −92.106 | 1.00 | 24.88 | C |
| ATOM | 663 | CD | LYS | | 97 | 33.819 | 3.238 | −90.631 | 1.00 | 23.47 | C |
| ATOM | 664 | CE | LYS | | 97 | 35.077 | 3.964 | −90.190 | 1.00 | 25.08 | C |
| ATOM | 665 | NZ | LYS | | 97 | 35.420 | 3.637 | −88.779 | 1.00 | 25.12 | N |
| ATOM | 666 | C | LYS | | 97 | 31.581 | 1.229 | −94.483 | 1.00 | 18.99 | C |
| ATOM | 667 | O | LYS | | 97 | 32.321 | 0.279 | −94.714 | 1.00 | 18.62 | O |
| ATOM | 668 | N | SER | | 98 | 30.255 | 1.154 | −94.565 | 1.00 | 17.76 | N |
| ATOM | 669 | CA | SER | | 98 | 29.586 | −0.085 | −94.969 | 1.00 | 20.00 | C |
| ATOM | 670 | CB | SER | | 98 | 28.123 | −0.078 | −94.523 | 1.00 | 19.57 | C |
| ATOM | 671 | OG | SER | | 98 | 27.376 | 0.876 | −95.268 | 1.00 | 19.19 | O |
| ATOM | 672 | C | SER | | 98 | 29.625 | −0.250 | −96.488 | 1.00 | 21.18 | C |
| ATOM | 673 | O | SER | | 98 | 29.393 | −1.340 | −97.013 | 1.00 | 20.44 | O |
| ATOM | 674 | N | GLY | | 99 | 29.914 | 0.834 | −97.199 | 1.00 | 17.02 | N |
| ATOM | 675 | CA | GLY | | 99 | 29.943 | 0.748 | −98.645 | 1.00 | 18.61 | C |
| ATOM | 676 | C | GLY | | 99 | 28.561 | 0.990 | −99.232 | 1.00 | 19.63 | C |
| ATOM | 677 | O | GLY | | 99 | 28.396 | 1.031 | −100.450 | 1.00 | 20.28 | O |
| ATOM | 678 | N | GLU | | 100 | 27.560 | 1.136 | −98.369 | 1.00 | 23.74 | N |
| ATOM | 679 | CA | GLU | | 100 | 26.204 | 1.408 | −98.832 | 1.00 | 26.69 | C |
| ATOM | 680 | CB | GLU | | 100 | 25.252 | 1.579 | −97.647 | 1.00 | 37.56 | C |
| ATOM | 681 | CG | GLU | | 100 | 24.914 | 0.302 | −96.905 | 1.00 | 45.64 | C |
| ATOM | 682 | CD | GLU | | 100 | 24.090 | 0.564 | −95.656 | 1.00 | 47.74 | C |
| ATOM | 683 | OE1 | GLU | | 100 | 24.656 | 1.051 | −94.652 | 1.00 | 49.67 | O |
| ATOM | 684 | OE2 | GLU | | 100 | 22.872 | 0.291 | −95.685 | 1.00 | 52.07 | O |
| ATOM | 685 | C | GLU | | 100 | 26.191 | 2.701 | −99.656 | 1.00 | 27.50 | C |
| ATOM | 686 | O | GLU | | 100 | 25.443 | 2.823 | −100.622 | 1.00 | 28.63 | O |
| ATOM | 687 | N | GLU | | 101 | 27.026 | 3.661 | −99.266 | 1.00 | 30.63 | N |
| ATOM | 688 | CA | GLU | | 101 | 27.092 | 4.952 | −99.950 | 1.00 | 33.03 | C |
| ATOM | 689 | CB | GLU | | 101 | 26.185 | 5.965 | −99.239 | 1.00 | 40.02 | C |
| ATOM | 690 | CG | GLU | | 101 | 24.700 | 5.743 | −99.442 | 1.00 | 43.12 | C |
| ATOM | 691 | CD | GLU | | 101 | 24.206 | 6.365 | −100.726 | 1.00 | 44.86 | C |
| ATOM | 692 | OE1 | GLU | | 101 | 24.738 | 6.019 | −101.800 | 1.00 | 48.15 | O |
| ATOM | 693 | OE2 | GLU | | 101 | 23.289 | 7.210 | −100.663 | 1.00 | 47.74 | O |
| ATOM | 694 | C | GLU | | 101 | 28.516 | 5.488 | −99.951 | 1.00 | 32.43 | C |
| ATOM | 695 | O | GLU | | 101 | 29.223 | 5.391 | −98.947 | 1.00 | 34.71 | O |
| ATOM | 696 | N | ASP | | 102 | 28.953 | 6.054 | −101.068 | 1.00 | 22.76 | N |
| ATOM | 697 | CA | ASP | | 102 | 30.303 | 6.595 | −101.088 | 1.00 | 23.62 | C |
| ATOM | 698 | CB | ASP | | 102 | 30.933 | 6.463 | −102.479 | 1.00 | 73.61 | C |
| ATOM | 699 | CG | ASP | | 102 | 30.014 | 6.907 | −103.582 | 1.00 | 76.83 | C |
| ATOM | 700 | OD1 | ASP | | 102 | 30.385 | 6.733 | −104.763 | 1.00 | 82.29 | O |
| ATOM | 701 | OD2 | ASP | | 102 | 28.924 | 7.428 | −103.272 | 1.00 | 80.71 | O |
| ATOM | 702 | C | ASP | | 102 | 30.309 | 8.047 | −100.610 | 1.00 | 21.48 | C |
| ATOM | 703 | O | ASP | | 102 | 29.311 | 8.771 | −100.725 | 1.00 | 19.79 | O |
| ATOM | 704 | N | PHE | | 103 | 31.439 | 8.444 | −100.048 | 1.00 | 24.32 | N |
| ATOM | 705 | CA | PHE | | 103 | 31.640 | 9.785 | −99.516 | 1.00 | 23.29 | C |
| ATOM | 706 | CB | PHE | | 103 | 33.129 | 9.982 | −99.212 | 1.00 | 25.00 | C |
| ATOM | 707 | CG | PHE | | 103 | 33.470 | 11.348 | −98.691 | 1.00 | 26.07 | C |
| ATOM | 708 | CD1 | PHE | | 103 | 33.355 | 11.637 | −97.341 | 1.00 | 26.61 | C |
| ATOM | 709 | CD2 | PHE | | 103 | 33.879 | 12.351 | −99.561 | 1.00 | 27.96 | C |
| ATOM | 710 | CE1 | PHE | | 103 | 33.639 | 12.903 | −96.861 | 1.00 | 27.03 | C |
| ATOM | 711 | CE2 | PHE | | 103 | 34.166 | 13.625 | −99.090 | 1.00 | 27.71 | C |
| ATOM | 712 | CZ | PHE | | 103 | 34.045 | 13.899 | −97.736 | 1.00 | 26.49 | C |
| ATOM | 713 | C | PHE | | 103 | 31.155 | 10.904 | −100.447 | 1.00 | 21.97 | C |
| ATOM | 714 | O | PHE | | 103 | 30.372 | 11.765 | −100.044 | 1.00 | 20.10 | O |
| ATOM | 715 | N | GLU | | 104 | 31.615 | 10.880 | −101.696 | 1.00 | 25.36 | N |
| ATOM | 716 | CA | GLU | | 104 | 31.262 | 11.917 | −102.664 | 1.00 | 27.12 | C |
| ATOM | 717 | CB | GLU | | 104 | 31.940 | 11.635 | −104.013 | 1.00 | 26.47 | C |
| ATOM | 718 | CG | GLU | | 104 | 33.460 | 11.565 | −103.932 | 1.00 | 27.20 | C |
| ATOM | 719 | CD | GLU | | 104 | 33.979 | 10.187 | −103.534 | 1.00 | 28.18 | C |
| ATOM | 720 | OE1 | GLU | | 104 | 33.249 | 9.411 | −102.873 | 1.00 | 28.08 | O |
| ATOM | 721 | OE2 | GLU | | 104 | 35.138 | 9.890 | −103.877 | 1.00 | 31.69 | O |
| ATOM | 722 | C | GLU | | 104 | 29.767 | 12.107 | −102.874 | 1.00 | 26.72 | C |
| ATOM | 723 | O | GLU | | 104 | 29.291 | 13.234 | −103.054 | 1.00 | 27.16 | O |
| ATOM | 724 | N | SER | | 105 | 29.032 | 11.003 | −102.866 | 1.00 | 22.30 | N |
| ATOM | 725 | CA | SER | | 105 | 27.592 | 11.042 | −103.066 | 1.00 | 24.72 | C |
| ATOM | 726 | CB | SER | | 105 | 27.053 | 9.624 | −103.223 | 1.00 | 30.44 | C |
| ATOM | 727 | OG | SER | | 105 | 25.644 | 9.625 | −103.319 | 1.00 | 32.49 | O |
| ATOM | 728 | C | SER | | 105 | 26.884 | 11.737 | −101.905 | 1.00 | 23.59 | C |
| ATOM | 729 | O | SER | | 105 | 26.000 | 12.573 | −102.107 | 1.00 | 23.88 | O |
| ATOM | 730 | N | LEU | | 106 | 27.266 | 11.383 | −100.686 | 1.00 | 18.90 | N |
| ATOM | 731 | CA | LEU | | 106 | 26.663 | 11.989 | −99.501 | 1.00 | 17.51 | C |
| ATOM | 732 | CB | LEU | | 106 | 27.067 | 11.206 | −98.249 | 1.00 | 16.55 | C |
| ATOM | 733 | CG | LEU | | 106 | 26.564 | 9.757 | −98.188 | 1.00 | 17.23 | C |
| ATOM | 734 | CD1 | LEU | | 106 | 27.020 | 9.107 | −96.890 | 1.00 | 15.14 | C |
| ATOM | 735 | CD2 | LEU | | 106 | 25.037 | 9.746 | −98.288 | 1.00 | 16.78 | C |
| ATOM | 736 | C | LEU | | 106 | 27.083 | 13.459 | −99.362 | 1.00 | 17.57 | C |
| ATOM | 737 | O | LEU | | 106 | 26.309 | 14.286 | −98.902 | 1.00 | 18.18 | O |
| ATOM | 738 | N | ALA | | 107 | 28.307 | 13.788 | −99.756 | 1.00 | 22.43 | N |
| ATOM | 739 | CA | ALA | | 107 | 28.756 | 15.170 | −99.656 | 1.00 | 23.57 | C |
| ATOM | 740 | CB | ALA | | 107 | 30.255 | 15.267 | −99.966 | 1.00 | 17.75 | C |
| ATOM | 741 | C | ALA | | 107 | 27.957 | 16.056 | −100.618 | 1.00 | 24.31 | C |
| ATOM | 742 | O | ALA | | 107 | 27.499 | 17.141 | −100.246 | 1.00 | 23.20 | O |
| ATOM | 743 | N | SER | | 108 | 27.790 | 15.582 | −101.851 | 1.00 | 22.34 | N |
| ATOM | 744 | CA | SER | | 108 | 27.061 | 16.328 | −102.869 | 1.00 | 23.59 | C |
| ATOM | 745 | CB | SER | | 108 | 27.078 | 15.572 | −104.202 | 1.00 | 26.85 | C |
| ATOM | 746 | OG | SER | | 108 | 28.398 | 15.411 | −104.689 | 1.00 | 31.79 | O |
| ATOM | 747 | C | SER | | 108 | 25.615 | 16.541 | −102.447 | 1.00 | 24.10 | C |
| ATOM | 748 | O | SER | | 108 | 25.031 | 17.596 | −102.684 | 1.00 | 24.01 | O |
| ATOM | 749 | N | GLN | | 109 | 25.046 | 15.527 | −101.812 | 1.00 | 23.12 | N |
| ATOM | 750 | CA | GLN | | 109 | 23.663 | 15.579 | −101.371 | 1.00 | 25.11 | C |
| ATOM | 751 | CB | GLN | | 109 | 23.096 | 14.167 | −101.267 | 1.00 | 30.14 | C |
| ATOM | 752 | CG | GLN | | 109 | 22.803 | 13.476 | −102.572 | 1.00 | 30.70 | C |
| ATOM | 753 | CD | GLN | | 109 | 22.390 | 12.039 | −102.337 | 1.00 | 33.54 | C |
| ATOM | 754 | OE1 | GLN | | 109 | 23.236 | 11.156 | −102.166 | 1.00 | 34.39 | O |
| ATOM | 755 | NE2 | GLN | | 109 | 21.089 | 11.800 | −102.298 | 1.00 | 32.03 | N |
| ATOM | 756 | C | GLN | | 109 | 23.384 | 16.258 | −100.042 | 1.00 | 24.46 | C |
| ATOM | 757 | O | GLN | | 109 | 22.371 | 16.930 | −99.901 | 1.00 | 23.21 | O |
| ATOM | 758 | N | PHE | | 110 | 24.280 | 16.102 | −99.075 | 1.00 | 26.35 | N |
| ATOM | 759 | CA | PHE | | 110 | 23.999 | 16.618 | −97.749 | 1.00 | 27.07 | C |
| ATOM | 760 | CB | PHE | | 110 | 23.757 | 15.422 | −96.824 | 1.00 | 23.12 | C |
| ATOM | 761 | CG | PHE | | 110 | 22.789 | 14.414 | −97.380 | 1.00 | 23.19 | C |
| ATOM | 762 | CD1 | PHE | | 110 | 23.205 | 13.120 | −97.678 | 1.00 | 22.81 | C |
| ATOM | 763 | CD2 | PHE | | 110 | 21.465 | 14.754 | −97.600 | 1.00 | 23.52 | C |
| ATOM | 764 | CE1 | PHE | | 110 | 22.309 | 12.181 | −98.184 | 1.00 | 26.02 | C |
| ATOM | 765 | CE2 | PHE | | 110 | 20.562 | 13.828 | −98.105 | 1.00 | 24.02 | C |
| ATOM | 766 | CZ | PHE | | 110 | 20.984 | 12.538 | −98.398 | 1.00 | 26.01 | C |
| ATOM | 767 | C | PHE | | 110 | 24.949 | 17.589 | −97.058 | 1.00 | 27.22 | C |
| ATOM | 768 | O | PHE | | 110 | 24.619 | 18.102 | −95.994 | 1.00 | 28.23 | O |
| ATOM | 769 | N | SER | | 111 | 26.111 | 17.856 | −97.628 | 1.00 | 21.97 | N |
| ATOM | 770 | CA | SER | | 111 | 27.025 | 18.779 | −96.970 | 1.00 | 22.53 | C |
| ATOM | 771 | CB | SER | | 111 | 28.380 | 18.773 | −97.654 | 1.00 | 17.83 | C |
| ATOM | 772 | OG | SER | | 111 | 29.249 | 19.688 | −97.017 | 1.00 | 19.51 | O |
| ATOM | 773 | C | SER | | 111 | 26.481 | 20.215 | −96.946 | 1.00 | 23.80 | C |
| ATOM | 774 | O | SER | | 111 | 25.986 | 20.712 | −97.958 | 1.00 | 23.31 | O |
| ATOM | 775 | N | ASP | | 112 | 26.575 | 20.865 | −95.785 | 1.00 | 29.48 | N |
| ATOM | 776 | CA | ASP | | 112 | 26.108 | 22.239 | −95.642 | 1.00 | 31.90 | C |
| ATOM | 777 | CB | ASP | | 112 | 25.685 | 22.526 | −94.200 | 1.00 | 22.62 | C |
| ATOM | 778 | CG | ASP | | 112 | 24.300 | 22.004 | −93.886 | 1.00 | 24.75 | C |
| ATOM | 779 | OD1 | ASP | | 112 | 23.367 | 22.343 | −94.639 | 1.00 | 24.54 | O |
| ATOM | 780 | OD2 | ASP | | 112 | 24.135 | 21.261 | −92.887 | 1.00 | 21.79 | O |
| ATOM | 781 | C | ASP | | 112 | 27.188 | 23.223 | −96.068 | 1.00 | 31.82 | C |
| ATOM | 782 | O | ASP | | 112 | 27.185 | 24.385 | −95.664 | 1.00 | 34.26 | O |
| ATOM | 783 | N | CYS | | 113 | 28.124 | 22.746 | −96.877 | 1.00 | 31.92 | N |
| ATOM | 784 | CA | CYS | | 113 | 29.192 | 23.594 | −97.383 | 1.00 | 31.08 | C |
| ATOM | 785 | CB | CYS | | 113 | 30.562 | 22.952 | −97.160 | 1.00 | 25.22 | C |
| ATOM | 786 | SG | CYS | | 113 | 31.955 | 23.975 | −97.700 | 1.00 | 27.72 | S |
| ATOM | 787 | C | CYS | | 113 | 28.949 | 23.751 | −98.869 | 1.00 | 31.17 | C |
| ATOM | 788 | O | CYS | | 113 | 28.473 | 22.822 | −99.525 | 1.00 | 30.96 | O |
| ATOM | 789 | N | SER | | 114 | 29.263 | 24.927 | −99.397 | 1.00 | 28.80 | N |
| ATOM | 790 | CA | SER | | 114 | 29.087 | 25.191 | −100.816 | 1.00 | 29.82 | C |
| ATOM | 791 | CB | SER | | 114 | 29.491 | 26.633 | −101.140 | 1.00 | 38.43 | C |
| ATOM | 792 | OG | SER | | 114 | 30.796 | 26.921 | −100.661 | 1.00 | 41.03 | O |
| ATOM | 793 | C | SER | | 114 | 29.936 | 24.224 | −101.635 | 1.00 | 29.28 | C |
| ATOM | 794 | O | SER | | 114 | 29.662 | 23.999 | −102.820 | 1.00 | 28.73 | O |
| ATOM | 795 | N | SER | | 115 | 30.960 | 23.648 | −101.001 | 1.00 | 24.09 | N |
| ATOM | 796 | CA | SER | | 115 | 31.844 | 22.706 | −101.684 | 1.00 | 23.71 | C |
| ATOM | 797 | CB | SER | | 115 | 33.088 | 22.418 | −100.833 | 1.00 | 27.10 | C |
| ATOM | 798 | OG | SER | | 115 | 32.748 | 21.719 | −99.650 | 1.00 | 27.68 | O |
| ATOM | 799 | C | SER | | 115 | 31.129 | 21.397 | −102.025 | 1.00 | 21.99 | C |
| ATOM | 800 | O | SER | | 115 | 31.716 | 20.492 | −102.626 | 1.00 | 21.19 | O |
| ATOM | 801 | N | ALA | | 116 | 29.862 | 21.294 | −101.640 | 1.00 | 23.30 | N |
| ATOM | 802 | CA | ALA | | 116 | 29.076 | 20.106 | −101.952 | 1.00 | 22.38 | C |
| ATOM | 803 | CB | ALA | | 116 | 27.659 | 20.232 | −101.355 | 1.00 | 20.06 | C |
| ATOM | 804 | C | ALA | | 116 | 28.992 | 19.967 | −103.482 | 1.00 | 24.14 | C |
| ATOM | 805 | O | ALA | | 116 | 29.043 | 18.856 | −104.022 | 1.00 | 20.21 | O |
| ATOM | 806 | N | LYS | | 117 | 28.858 | 21.102 | −104.172 | 1.00 | 18.10 | N |
| ATOM | 807 | CA | LYS | | 117 | 28.768 | 21.109 | −105.639 | 1.00 | 19.43 | C |
| ATOM | 808 | CB | LYS | | 117 | 28.581 | 22.544 | −106.145 | 1.00 | 40.55 | C |
| ATOM | 809 | CG | LYS | | 117 | 27.420 | 23.281 | −105.499 | 1.00 | 46.69 | C |
| ATOM | 810 | CD | LYS | | 117 | 26.074 | 22.787 | −106.011 | 1.00 | 48.91 | C |
| ATOM | 811 | CE | LYS | | 117 | 24.919 | 23.335 | −105.167 | 1.00 | 51.75 | C |
| ATOM | 812 | NZ | LYS | | 117 | 24.908 | 22.757 | −103.786 | 1.00 | 49.86 | N |
| ATOM | 813 | C | LYS | | 117 | 30.012 | 20.500 | −106.299 | 1.00 | 18.45 | C |
| ATOM | 814 | O | LYS | | 117 | 30.001 | 20.193 | −107.484 | 1.00 | 16.93 | O |
| ATOM | 815 | N | ALA | | 118 | 31.091 | 20.359 | −105.535 | 1.00 | 23.21 | N |
| ATOM | 816 | CA | ALA | | 118 | 32.321 | 19.767 | −106.055 | 1.00 | 24.27 | C |
| ATOM | 817 | CB | ALA | | 118 | 33.497 | 20.735 | −105.894 | 1.00 | 20.75 | C |
| ATOM | 818 | C | ALA | | 118 | 32.609 | 18.471 | −105.298 | 1.00 | 24.90 | C |
| ATOM | 819 | O | ALA | | 118 | 33.764 | 18.111 | −105.087 | 1.00 | 24.98 | O |
| ATOM | 820 | N | ARG | | 119 | 31.547 | 17.787 | −104.886 | 1.00 | 20.58 | N |
| ATOM | 821 | CA | ARG | | 119 | 31.671 | 16.532 | −104.143 | 1.00 | 23.45 | C |
| ATOM | 822 | CB | ARG | | 119 | 32.263 | 15.435 | −105.030 | 1.00 | 66.29 | C |
| ATOM | 823 | CG | ARG | | 119 | 31.285 | 14.870 | −106.044 | 1.00 | 69.42 | C |
| ATOM | 824 | CD | ARG | | 119 | 31.987 | 14.071 | −107.134 | 1.00 | 73.46 | C |
| ATOM | 825 | NE | ARG | | 119 | 33.056 | 14.853 | −107.746 | 1.00 | 79.80 | N |
| ATOM | 826 | CZ | ARG | | 119 | 34.295 | 14.933 | −107.268 | 1.00 | 79.85 | C |
| ATOM | 827 | NH1 | ARG | | 119 | 34.631 | 14.265 | −106.171 | 1.00 | 81.46 | N |
| ATOM | 828 | NH2 | ARG | | 119 | 35.194 | 15.705 | −107.868 | 1.00 | 75.54 | N |
| ATOM | 829 | C | ARG | | 119 | 32.517 | 16.698 | −102.893 | 1.00 | 22.59 | C |
| ATOM | 830 | O | ARG | | 119 | 33.232 | 15.785 | −102.490 | 1.00 | 20.53 | O |
| ATOM | 831 | N | GLY | | 120 | 32.433 | 17.879 | −102.290 | 1.00 | 24.63 | N |
| ATOM | 832 | CA | GLY | | 120 | 33.168 | 18.135 | −101.066 | 1.00 | 25.04 | C |
| ATOM | 833 | C | GLY | | 120 | 34.628 | 18.504 | −101.198 | 1.00 | 25.79 | C |
| ATOM | 834 | O | GLY | | 120 | 35.252 | 18.875 | −100.208 | 1.00 | 25.08 | O |
| ATOM | 835 | N | ASP | | 121 | 35.182 | 18.412 | −102.402 | 1.00 | 23.84 | N |
| ATOM | 836 | CA | ASP | | 121 | 36.590 | 18.753 | −102.606 | 1.00 | 23.82 | C |
| ATOM | 837 | CB | ASP | | 121 | 37.008 | 18.467 | −104.052 | 1.00 | 29.84 | C |
| ATOM | 838 | CG | ASP | | 121 | 38.412 | 18.948 | −104.352 | 1.00 | 30.81 | C |
| ATOM | 839 | OD1 | ASP | | 121 | 39.327 | 18.641 | −103.554 | 1.00 | 28.96 | O |
| ATOM | 840 | OD2 | ASP | | 121 | 38.604 | 19.627 | −105.388 | 1.00 | 29.97 | O |
| ATOM | 841 | C | ASP | | 121 | 36.881 | 20.219 | −102.283 | 1.00 | 25.41 | C |
| ATOM | 842 | O | ASP | | 121 | 36.184 | 21.127 | −102.750 | 1.00 | 24.07 | O |
| ATOM | 843 | N | LEU | | 122 | 37.920 | 20.434 | −101.484 | 1.00 | 27.52 | N |
| ATOM | 844 | CA | LEU | | 122 | 38.321 | 21.774 | −101.083 | 1.00 | 29.03 | C |
| ATOM | 845 | CB | LEU | | 122 | 38.542 | 21.822 | −99.573 | 1.00 | 23.19 | C |
| ATOM | 846 | CG | LEU | | 122 | 37.317 | 21.590 | −98.688 | 1.00 | 23.30 | C |
| ATOM | 847 | CD1 | LEU | | 122 | 37.763 | 21.498 | −97.227 | 1.00 | 21.90 | C |
| ATOM | 848 | CD2 | LEU | | 122 | 36.328 | 22.728 | −98.878 | 1.00 | 23.14 | C |
| ATOM | 849 | C | LEU | | 122 | 39.599 | 22.227 | −101.777 | 1.00 | 30.09 | C |
| ATOM | 850 | O | LEU | | 122 | 40.024 | 23.367 | −101.611 | 1.00 | 30.44 | O |
| ATOM | 851 | N | GLY | | 123 | 40.210 | 21.341 | −102.555 | 1.00 | 32.59 | N |
| ATOM | 852 | CA | GLY | | 123 | 41.450 | 21.696 | −103.216 | 1.00 | 33.34 | C |
| ATOM | 853 | C | GLY | | 123 | 42.590 | 21.492 | −102.240 | 1.00 | 34.46 | C |
| ATOM | 854 | O | GLY | | 123 | 42.362 | 21.066 | −101.105 | 1.00 | 35.29 | O |
| ATOM | 855 | N | ALA | | 124 | 43.812 | 21.787 | −102.663 | 1.00 | 27.21 | N |
| ATOM | 856 | CA | ALA | | 124 | 44.972 | 21.619 | −101.796 | 1.00 | 28.46 | C |
| ATOM | 857 | CB | ALA | | 124 | 46.239 | 21.428 | −102.629 | 1.00 | 25.86 | C |
| ATOM | 858 | C | ALA | | 124 | 45.133 | 22.821 | −100.874 | 1.00 | 29.68 | C |
| ATOM | 859 | O | ALA | | 124 | 44.654 | 23.915 | −101.176 | 1.00 | 30.17 | O |
| ATOM | 860 | N | PHE | | 125 | 45.798 | 22.608 | −99.745 | 1.00 | 29.96 | N |
| ATOM | 861 | CA | PHE | | 125 | 46.018 | 23.677 | −98.785 | 1.00 | 30.00 | C |
| ATOM | 862 | CB | PHE | | 125 | 44.728 | 23.953 | −97.983 | 1.00 | 32.26 | C |
| ATOM | 863 | CG | PHE | | 125 | 44.198 | 22.757 | −97.215 | 1.00 | 32.59 | C |
| ATOM | 864 | CD1 | PHE | | 125 | 44.777 | 22.365 | −96.020 | 1.00 | 32.40 | C |
| ATOM | 865 | CD2 | PHE | | 125 | 43.101 | 22.042 | −97.685 | 1.00 | 32.88 | C |
| ATOM | 866 | CE1 | PHE | | 125 | 44.275 | 21.281 | −95.302 | 1.00 | 31.50 | C |
| ATOM | 867 | CE2 | PHE | | 125 | 42.593 | 20.959 | −96.974 | 1.00 | 32.77 | C |
| ATOM | 868 | CZ | PHE | | 125 | 43.183 | 20.579 | −95.777 | 1.00 | 31.03 | C |
| ATOM | 869 | C | PHE | | 125 | 47.175 | 23.327 | −97.861 | 1.00 | 29.21 | C |
| ATOM | 870 | O | PHE | | 125 | 47.582 | 22.164 | −97.766 | 1.00 | 30.99 | O |
| ATOM | 871 | N | SER | | 126 | 47.728 | 24.336 | −97.201 | 1.00 | 30.91 | N |
| ATOM | 872 | CA | SER | | 126 | 48.839 | 24.112 | −96.280 | 1.00 | 31.80 | C |
| ATOM | 873 | CB | SER | | 126 | 50.058 | 24.944 | −96.692 | 1.00 | 48.97 | C |
| ATOM | 874 | OG | SER | | 126 | 49.783 | 26.329 | −96.585 | 1.00 | 50.37 | O |
| ATOM | 875 | C | SER | | 126 | 48.381 | 24.522 | −94.894 | 1.00 | 30.73 | C |
| ATOM | 876 | O | SER | | 126 | 47.270 | 25.039 | −94.731 | 1.00 | 29.97 | O |
| ATOM | 877 | N | ARG | | 127 | 49.217 | 24.286 | −93.891 | 1.00 | 36.37 | N |
| ATOM | 878 | CA | ARG | | 127 | 48.849 | 24.660 | −92.535 | 1.00 | 37.50 | C |
| ATOM | 879 | CB | ARG | | 127 | 49.887 | 24.158 | −91.528 | 1.00 | 33.85 | C |
| ATOM | 880 | CG | ARG | | 127 | 49.804 | 22.673 | −91.216 | 1.00 | 33.05 | C |
| ATOM | 881 | CD | ARG | | 127 | 50.784 | 22.320 | −90.116 | 1.00 | 31.66 | C |
| ATOM | 882 | NE | ARG | | 127 | 50.733 | 20.912 | −89.722 | 1.00 | 31.07 | N |
| ATOM | 883 | CZ | ARG | | 127 | 49.761 | 20.363 | −88.994 | 1.00 | 30.21 | C |
| ATOM | 884 | NH1 | ARG | | 127 | 48.740 | 21.097 | −88.571 | 1.00 | 27.23 | N |
| ATOM | 885 | NH2 | ARG | | 127 | 49.825 | 19.078 | −88.674 | 1.00 | 30.30 | N |
| ATOM | 886 | C | ARG | | 127 | 48.738 | 26.174 | −92.442 | 1.00 | 40.02 | C |
| ATOM | 887 | O | ARG | | 127 | 49.542 | 26.900 | −93.027 | 1.00 | 40.22 | O |
| ATOM | 888 | N | GLY | | 128 | 47.729 | 26.645 | −91.718 | 1.00 | 49.10 | N |
| ATOM | 889 | CA | GLY | | 128 | 47.542 | 28.073 | −91.558 | 1.00 | 50.77 | C |
| ATOM | 890 | C | GLY | | 128 | 46.381 | 28.673 | −92.328 | 1.00 | 50.83 | C |
| ATOM | 891 | O | GLY | | 128 | 46.100 | 29.859 | −92.182 | 1.00 | 52.90 | O |
| ATOM | 892 | N | GLN | | 129 | 45.692 | 27.871 | −93.134 | 1.00 | 35.80 | N |
| ATOM | 893 | CA | GLN | | 129 | 44.578 | 28.389 | −93.922 | 1.00 | 34.37 | C |
| ATOM | 894 | CB | GLN | | 129 | 44.654 | 27.876 | −95.359 | 1.00 | 36.06 | C |
| ATOM | 895 | CG | GLN | | 129 | 46.045 | 27.850 | −95.969 | 1.00 | 38.81 | C |
| ATOM | 896 | CD | GLN | | 129 | 46.015 | 27.361 | −97.407 | 1.00 | 40.11 | C |
| ATOM | 897 | OE1 | GLN | | 129 | 47.040 | 26.980 | −97.972 | 1.00 | 41.71 | O |
| ATOM | 898 | NE2 | GLN | | 129 | 44.830 | 27.374 | −98.008 | 1.00 | 42.73 | N |
| ATOM | 899 | C | GLN | | 129 | 43.212 | 28.016 | −93.367 | 1.00 | 33.12 | C |
| ATOM | 900 | O | GLN | | 129 | 42.296 | 28.835 | −93.343 | 1.00 | 33.22 | O |
| ATOM | 901 | N | MET | | 130 | 43.081 | 26.766 | −92.939 | 1.00 | 31.88 | N |
| ATOM | 902 | CA | MET | | 130 | 41.821 | 26.252 | −92.417 | 1.00 | 31.07 | C |
| ATOM | 903 | CB | MET | | 130 | 41.676 | 24.772 | −92.807 | 1.00 | 32.84 | C |
| ATOM | 904 | CG | MET | | 130 | 41.901 | 24.480 | −94.290 | 1.00 | 34.76 | C |
| ATOM | 905 | SD | MET | | 130 | 40.640 | 25.172 | −95.377 | 1.00 | 37.86 | S |
| ATOM | 906 | CE | MET | | 130 | 41.211 | 24.584 | −96.966 | 1.00 | 37.27 | C |
| ATOM | 907 | C | MET | | 130 | 41.742 | 26.382 | −90.897 | 1.00 | 30.31 | C |
| ATOM | 908 | O | MET | | 130 | 42.746 | 26.653 | −90.237 | 1.00 | 30.11 | O |
| ATOM | 909 | N | GLN | | 131 | 40.542 | 26.200 | −90.353 | 1.00 | 32.53 | N |
| ATOM | 910 | CA | GLN | | 131 | 40.347 | 26.255 | −88.910 | 1.00 | 33.04 | C |
| ATOM | 911 | CB | GLN | | 131 | 38.870 | 26.066 | −88.577 | 1.00 | 36.19 | C |
| ATOM | 912 | CG | GLN | | 131 | 37.980 | 27.079 | −89.287 | 1.00 | 38.94 | C |
| ATOM | 913 | CD | GLN | | 131 | 36.508 | 26.907 | −88.967 | 1.00 | 40.59 | C |
| ATOM | 914 | OE1 | GLN | | 131 | 36.084 | 27.078 | −87.824 | 1.00 | 44.90 | O |
| ATOM | 915 | NE2 | GLN | | 131 | 35.718 | 26.562 | −89.980 | 1.00 | 41.02 | N |
| ATOM | 916 | C | GLN | | 131 | 41.195 | 25.120 | −88.345 | 1.00 | 32.61 | C |
| ATOM | 917 | O | GLN | | 131 | 41.216 | 24.012 | −88.895 | 1.00 | 32.04 | O |
| ATOM | 918 | N | LYS | | 132 | 41.899 | 25.410 | −87.256 | 1.00 | 29.66 | N |
| ATOM | 919 | CA | LYS | | 132 | 42.821 | 24.470 | −86.620 | 1.00 | 27.83 | C |
| ATOM | 920 | CB | LYS | | 132 | 43.259 | 25.030 | −85.257 | 1.00 | 25.86 | C |
| ATOM | 921 | CG | LYS | | 132 | 44.620 | 24.528 | −84.784 | 1.00 | 25.88 | C |
| ATOM | 922 | CD | LYS | | 132 | 45.722 | 24.992 | −85.715 | 1.00 | 24.67 | C |
| ATOM | 923 | CE | LYS | | 132 | 46.555 | 23.824 | −86.265 | 1.00 | 25.54 | C |
| ATOM | 924 | NZ | LYS | | 132 | 47.581 | 23.270 | −85.331 | 1.00 | 26.89 | N |
| ATOM | 925 | C | LYS | | 132 | 42.388 | 23.001 | −86.470 | 1.00 | 27.14 | C |
| ATOM | 926 | O | LYS | | 132 | 43.142 | 22.095 | −86.829 | 1.00 | 26.64 | O |
| ATOM | 927 | N | PRO | | 133 | 41.188 | 22.740 | −85.926 | 1.00 | 22.57 | N |
| ATOM | 928 | CD | PRO | | 133 | 40.154 | 23.659 | −85.419 | 1.00 | 31.97 | C |
| ATOM | 929 | CA | PRO | | 133 | 40.763 | 21.338 | −85.776 | 1.00 | 24.04 | C |
| ATOM | 930 | CB | PRO | | 133 | 39.349 | 21.463 | −85.200 | 1.00 | 33.19 | C |
| ATOM | 931 | CG | PRO | | 133 | 39.394 | 22.779 | −84.455 | 1.00 | 33.49 | C |
| ATOM | 932 | C | PRO | | 133 | 40.776 | 20.607 | −87.123 | 1.00 | 23.70 | C |
| ATOM | 933 | O | PRO | | 133 | 41.315 | 19.504 | −87.246 | 1.00 | 23.13 | O |
| ATOM | 934 | N | PHE | | 134 | 40.192 | 21.247 | −88.132 | 1.00 | 27.49 | N |
| ATOM | 935 | CA | PHE | | 134 | 40.128 | 20.689 | −89.483 | 1.00 | 26.00 | C |
| ATOM | 936 | CB | PHE | | 134 | 39.431 | 21.675 | −90.413 | 1.00 | 24.79 | C |
| ATOM | 937 | CG | PHE | | 134 | 38.965 | 21.077 | −91.711 | 1.00 | 22.98 | C |
| ATOM | 938 | CD1 | PHE | | 134 | 37.680 | 20.567 | −91.828 | 1.00 | 20.76 | C |
| ATOM | 939 | CD2 | PHE | | 134 | 39.791 | 21.066 | −92.825 | 1.00 | 22.54 | C |
| ATOM | 940 | CE1 | PHE | | 134 | 37.219 | 20.058 | −93.040 | 1.00 | 21.87 | C |
| ATOM | 941 | CE2 | PHE | | 134 | 39.340 | 20.559 | −94.042 | 1.00 | 21.53 | C |
| ATOM | 942 | CZ | PHE | | 134 | 38.053 | 20.056 | −94.148 | 1.00 | 21.75 | C |
| ATOM | 943 | C | PHE | | 134 | 41.544 | 20.448 | −89.994 | 1.00 | 25.38 | C |
| ATOM | 944 | O | PHE | | 134 | 41.860 | 19.375 | −90.520 | 1.00 | 24.29 | O |
| ATOM | 945 | N | GLU | | 135 | 42.393 | 21.462 | −89.839 | 1.00 | 25.30 | N |
| ATOM | 946 | CA | GLU | | 135 | 43.775 | 21.375 | −90.289 | 1.00 | 24.08 | C |
| ATOM | 947 | CB | GLU | | 135 | 44.534 | 22.653 | −89.931 | 1.00 | 27.17 | C |
| ATOM | 948 | CG | GLU | | 135 | 46.026 | 22.577 | −90.198 | 1.00 | 29.36 | C |
| ATOM | 949 | CD | GLU | | 135 | 46.743 | 23.855 | −89.789 | 1.00 | 32.18 | C |
| ATOM | 950 | OE1 | GLU | | 135 | 46.274 | 24.947 | −90.177 | 1.00 | 32.15 | O |
| ATOM | 951 | OE2 | GLU | | 135 | 47.768 | 23.764 | −89.084 | 1.00 | 34.46 | O |
| ATOM | 952 | C | GLU | | 135 | 44.506 | 20.185 | −89.686 | 1.00 | 23.82 | C |
| ATOM | 953 | O | GLU | | 135 | 45.047 | 19.344 | −90.408 | 1.00 | 21.02 | O |
| ATOM | 954 | N | ASP | | 136 | 44.532 | 20.125 | −88.362 | 1.00 | 18.96 | N |
| ATOM | 955 | CA | ASP | | 136 | 45.213 | 19.040 | −87.667 | 1.00 | 18.64 | C |
| ATOM | 956 | CB | ASP | | 136 | 45.053 | 19.182 | −86.148 | 1.00 | 33.63 | C |
| ATOM | 957 | CG | ASP | | 136 | 45.805 | 20.388 | −85.594 | 1.00 | 33.64 | C |
| ATOM | 958 | OD1 | ASP | | 136 | 46.928 | 20.663 | −86.083 | 1.00 | 33.59 | O |
| ATOM | 959 | OD2 | ASP | | 136 | 45.280 | 21.051 | −84.672 | 1.00 | 34.58 | O |
| ATOM | 960 | C | ASP | | 136 | 44.690 | 17.687 | −88.122 | 1.00 | 17.27 | C |
| ATOM | 961 | O | ASP | | 136 | 45.468 | 16.772 | −88.387 | 1.00 | 17.39 | O |
| ATOM | 962 | N | ALA | | 137 | 43.373 | 17.564 | −88.227 | 1.00 | 23.61 | N |
| ATOM | 963 | CA | ALA | | 137 | 42.791 | 16.300 | −88.663 | 1.00 | 23.57 | C |
| ATOM | 964 | CB | ALA | | 137 | 41.257 | 16.388 | −88.629 | 1.00 | 24.33 | C |
| ATOM | 965 | C | ALA | | 137 | 43.288 | 15.999 | −90.081 | 1.00 | 23.57 | C |
| ATOM | 966 | O | ALA | | 137 | 43.760 | 14.897 | −90.369 | 1.00 | 22.96 | O |
| ATOM | 967 | N | SER | | 138 | 43.208 | 16.997 | −90.954 | 1.00 | 24.89 | N |
| ATOM | 968 | CA | SER | | 138 | 43.639 | 16.841 | −92.345 | 1.00 | 25.57 | C |
| ATOM | 969 | CB | SER | | 138 | 43.480 | 18.156 | −93.101 | 1.00 | 19.03 | C |
| ATOM | 970 | OG | SER | | 138 | 42.123 | 18.530 | −93.165 | 1.00 | 17.05 | O |
| ATOM | 971 | C | SER | | 138 | 45.064 | 16.356 | −92.506 | 1.00 | 26.91 | C |
| ATOM | 972 | O | SER | | 138 | 45.321 | 15.430 | −93.277 | 1.00 | 25.54 | O |
| ATOM | 973 | N | PHE | | 139 | 45.991 | 16.977 | −91.782 | 1.00 | 24.01 | N |
| ATOM | 974 | CA | PHE | | 139 | 47.400 | 16.605 | −91.861 | 1.00 | 25.68 | C |
| ATOM | 975 | CB | PHE | | 139 | 48.286 | 17.766 | −91.391 | 1.00 | 33.43 | C |
| ATOM | 976 | CG | PHE | | 139 | 48.501 | 18.818 | −92.442 | 1.00 | 33.62 | C |
| ATOM | 977 | CD1 | PHE | | 139 | 47.519 | 19.754 | −92.718 | 1.00 | 33.08 | C |
| ATOM | 978 | CD2 | PHE | | 139 | 49.657 | 18.821 | −93.206 | 1.00 | 35.11 | C |
| ATOM | 979 | CE1 | PHE | | 139 | 47.682 | 20.676 | −93.742 | 1.00 | 34.16 | C |
| ATOM | 980 | CE2 | PHE | | 139 | 49.827 | 19.740 | −94.234 | 1.00 | 34.09 | C |
| ATOM | 981 | CZ | PHE | | 139 | 48.836 | 20.666 | −94.502 | 1.00 | 33.18 | C |
| ATOM | 982 | C | PHE | | 139 | 47.789 | 15.334 | −91.105 | 1.00 | 26.85 | C |
| ATOM | 983 | O | PHE | | 139 | 48.869 | 14.787 | −91.322 | 1.00 | 27.55 | O |
| ATOM | 984 | N | ALA | | 140 | 46.924 | 14.873 | −90.210 | 1.00 | 26.06 | N |
| ATOM | 985 | CA | ALA | | 140 | 47.209 | 13.660 | −89.461 | 1.00 | 25.55 | C |
| ATOM | 986 | CB | ALA | | 140 | 46.531 | 13.716 | −88.101 | 1.00 | 19.65 | C |
| ATOM | 987 | C | ALA | | 140 | 46.704 | 12.463 | −90.267 | 1.00 | 25.79 | C |
| ATOM | 988 | O | ALA | | 140 | 47.124 | 11.322 | −90.049 | 1.00 | 24.80 | O |
| ATOM | 989 | N | LEU | | 141 | 45.806 | 12.729 | −91.211 | 1.00 | 30.05 | N |
| ATOM | 990 | CA | LEU | | 141 | 45.269 | 11.669 | −92.056 | 1.00 | 30.74 | C |
| ATOM | 991 | CB | LEU | | 141 | 44.015 | 12.152 | −92.794 | 1.00 | 25.73 | C |
| ATOM | 992 | CG | LEU | | 141 | 42.714 | 12.337 | −92.007 | 1.00 | 25.32 | C |
| ATOM | 993 | CD1 | LEU | | 141 | 41.685 | 13.045 | −92.878 | 1.00 | 22.31 | C |
| ATOM | 994 | CD2 | LEU | | 141 | 42.188 | 10.974 | −91.558 | 1.00 | 24.22 | C |
| ATOM | 995 | C | LEU | | 141 | 46.298 | 11.229 | −93.086 | 1.00 | 32.06 | C |
| ATOM | 996 | O | LEU | | 141 | 47.136 | 12.019 | −93.522 | 1.00 | 32.47 | O |
| ATOM | 997 | N | ARG | | 142 | 46.251 | 9.959 | −93.464 | 1.00 | 24.26 | N |
| ATOM | 998 | CA | ARG | | 142 | 47.152 | 9.464 | −94.493 | 1.00 | 24.58 | C |
| ATOM | 999 | CB | ARG | | 142 | 47.529 | 8.008 | −94.216 | 1.00 | 91.42 | C |
| ATOM | 1000 | CG | ARG | | 142 | 48.475 | 7.856 | −93.033 | 1.00 | 91.49 | C |
| ATOM | 1001 | CD | ARG | | 142 | 48.876 | 6.410 | −92.811 | 1.00 | 95.06 | C |
| ATOM | 1002 | NE | ARG | | 142 | 47.814 | 5.629 | −92.184 | 1.00 | 97.55 | N |
| ATOM | 1003 | CZ | ARG | | 142 | 47.399 | 5.803 | −90.934 | 1.00 | 97.12 | C |
| ATOM | 1004 | NH1 | ARG | | 142 | 47.956 | 6.735 | −90.172 | 1.00 | 96.19 | N |
| ATOM | 1005 | NH2 | ARG | | 142 | 46.428 | 5.046 | −90.443 | 1.00 | 96.04 | N |
| ATOM | 1006 | C | ARG | | 142 | 46.355 | 9.597 | −95.787 | 1.00 | 25.25 | C |
| ATOM | 1007 | O | ARG | | 142 | 45.141 | 9.771 | −95.744 | 1.00 | 23.99 | O |
| ATOM | 1008 | N | THR | | 143 | 47.024 | 9.555 | −96.930 | 1.00 | 28.86 | N |
| ATOM | 1009 | CA | THR | | 143 | 46.321 | 9.673 | −98.199 | 1.00 | 28.89 | C |
| ATOM | 1010 | CB | THR | | 143 | 47.289 | 9.572 | −99.388 | 1.00 | 27.35 | C |
| ATOM | 1011 | OG1 | THR | | 143 | 48.181 | 10.690 | −99.370 | 1.00 | 31.19 | O |
| ATOM | 1012 | CG2 | THR | | 143 | 46.514 | 9.562 | −100.704 | 1.00 | 26.93 | C |
| ATOM | 1013 | C | THR | | 143 | 45.304 | 8.547 | −98.324 | 1.00 | 28.31 | C |
| ATOM | 1014 | O | THR | | 143 | 45.631 | 7.391 | −98.070 | 1.00 | 25.80 | O |
| ATOM | 1015 | N | GLY | | 144 | 44.076 | 8.890 | −98.706 | 1.00 | 21.78 | N |
| ATOM | 1016 | CA | GLY | | 144 | 43.038 | 7.886 | −98.866 | 1.00 | 22.57 | C |
| ATOM | 1017 | C | GLY | | 144 | 42.216 | 7.635 | −97.615 | 1.00 | 21.55 | C |
| ATOM | 1018 | O | GLY | | 144 | 41.121 | 7.070 | −97.685 | 1.00 | 22.10 | O |
| ATOM | 1019 | N | GLU | | 145 | 42.738 | 8.077 | −96.473 | 1.00 | 26.69 | N |
| ATOM | 1020 | CA | GLU | | 145 | 42.088 | 7.889 | −95.181 | 1.00 | 26.01 | C |
| ATOM | 1021 | CB | GLU | | 145 | 43.151 | 7.938 | −94.080 | 1.00 | 30.85 | C |
| ATOM | 1022 | CG | GLU | | 145 | 42.617 | 7.803 | −92.673 | 1.00 | 34.28 | C |
| ATOM | 1023 | CD | GLU | | 145 | 43.702 | 7.419 | −91.677 | 1.00 | 36.90 | C |
| ATOM | 1024 | OE1 | GLU | | 145 | 44.905 | 7.554 | −92.020 | 1.00 | 37.05 | O |
| ATOM | 1025 | OE2 | GLU | | 145 | 43.346 | 6.985 | −90.552 | 1.00 | 35.71 | O |
| ATOM | 1026 | C | GLU | | 145 | 40.946 | 8.863 | −94.864 | 1.00 | 24.84 | C |
| ATOM | 1027 | O | GLU | | 145 | 40.868 | 9.971 | −95.404 | 1.00 | 25.13 | O |
| ATOM | 1028 | N | MET | | 146 | 40.061 | 8.440 | −93.972 | 1.00 | 20.35 | N |
| ATOM | 1029 | CA | MET | | 146 | 38.920 | 9.256 | −93.596 | 1.00 | 20.47 | C |
| ATOM | 1030 | CB | MET | | 146 | 37.619 | 8.579 | −94.059 | 1.00 | 23.06 | C |
| ATOM | 1031 | CG | MET | | 146 | 36.365 | 9.414 | −93.810 | 1.00 | 21.37 | C |
| ATOM | 1032 | SD | MET | | 146 | 34.844 | 8.679 | −94.440 | 1.00 | 21.15 | S |
| ATOM | 1033 | CE | MET | | 146 | 34.428 | 7.537 | −93.095 | 1.00 | 20.79 | C |
| ATOM | 1034 | C | MET | | 146 | 38.885 | 9.506 | −92.091 | 1.00 | 20.26 | C |
| ATOM | 1035 | O | MET | | 146 | 39.256 | 8.648 | −91.288 | 1.00 | 17.10 | O |
| ATOM | 1036 | N | SER | | 147 | 38.425 | 10.691 | −91.716 | 1.00 | 17.48 | N |
| ATOM | 1037 | CA | SER | | 147 | 38.351 | 11.070 | −90.315 | 1.00 | 16.86 | C |
| ATOM | 1038 | CB | SER | | 147 | 38.440 | 12.589 | −90.177 | 1.00 | 16.80 | C |
| ATOM | 1039 | OG | SER | | 147 | 37.153 | 13.158 | −90.430 | 1.00 | 17.03 | O |
| ATOM | 1040 | C | SER | | 147 | 37.031 | 10.648 | −89.686 | 1.00 | 16.63 | C |
| ATOM | 1041 | O | SER | | 147 | 36.119 | 10.187 | −90.368 | 1.00 | 15.19 | O |
| ATOM | 1042 | N | GLY | | 148 | 36.953 | 10.817 | −88.371 | 1.00 | 19.57 | N |
| ATOM | 1043 | CA | GLY | | 148 | 35.722 | 10.565 | −87.654 | 1.00 | 21.69 | C |
| ATOM | 1044 | C | GLY | | 148 | 35.139 | 11.969 | −87.548 | 1.00 | 22.31 | C |
| ATOM | 1045 | O | GLY | | 148 | 35.592 | 12.869 | −88.271 | 1.00 | 21.73 | O |
| ATOM | 1046 | N | PRO | | 149 | 34.147 | 12.202 | −86.682 | 1.00 | 24.08 | N |
| ATOM | 1047 | CD | PRO | | 149 | 33.394 | 11.222 | −85.881 | 1.00 | 24.18 | C |
| ATOM | 1048 | CA | PRO | | 149 | 33.576 | 13.546 | −86.561 | 1.00 | 23.27 | C |
| ATOM | 1049 | CB | PRO | | 149 | 32.468 | 13.364 | −85.521 | 1.00 | 25.65 | C |
| ATOM | 1050 | CG | PRO | | 149 | 32.054 | 11.910 | −85.706 | 1.00 | 26.28 | C |
| ATOM | 1051 | C | PRO | | 149 | 34.628 | 14.556 | −86.103 | 1.00 | 23.78 | C |
| ATOM | 1052 | O | PRO | | 149 | 35.289 | 14.347 | −85.083 | 1.00 | 21.81 | O |
| ATOM | 1053 | N | VAL | | 150 | 34.779 | 15.645 | −86.857 | 1.00 | 19.30 | N |
| ATOM | 1054 | CA | VAL | | 150 | 35.749 | 16.702 | −86.526 | 1.00 | 19.47 | C |
| ATOM | 1055 | CB | VAL | | 150 | 36.761 | 16.918 | −87.673 | 1.00 | 19.26 | C |
| ATOM | 1056 | CG1 | VAL | | 150 | 37.640 | 18.144 | −87.386 | 1.00 | 17.57 | C |
| ATOM | 1057 | CG2 | VAL | | 150 | 37.631 | 15.683 | −87.826 | 1.00 | 20.26 | C |
| ATOM | 1058 | C | VAL | | 150 | 34.982 | 18.002 | −86.272 | 1.00 | 20.31 | C |
| ATOM | 1059 | O | VAL | | 150 | 34.231 | 18.469 | −87.132 | 1.00 | 19.82 | O |
| ATOM | 1060 | N | PHE | | 151 | 35.184 | 18.584 | −85.090 | 1.00 | 20.39 | N |
| ATOM | 1061 | CA | PHE | | 151 | 34.466 | 19.794 | −84.707 | 1.00 | 22.47 | C |
| ATOM | 1062 | CB | PHE | | 151 | 34.045 | 19.696 | −83.234 | 1.00 | 21.83 | C |
| ATOM | 1063 | CG | PHE | | 151 | 33.243 | 18.466 | −82.908 | 1.00 | 22.44 | C |
| ATOM | 1064 | CD1 | PHE | | 151 | 33.862 | 17.228 | −82.781 | 1.00 | 20.93 | C |
| ATOM | 1065 | CD2 | PHE | | 151 | 31.873 | 18.547 | −82.719 | 1.00 | 22.17 | C |
| ATOM | 1066 | CE1 | PHE | | 151 | 33.126 | 16.095 | −82.467 | 1.00 | 22.60 | C |
| ATOM | 1067 | CE2 | PHE | | 151 | 31.128 | 17.414 | −82.407 | 1.00 | 22.25 | C |
| ATOM | 1068 | CZ | PHE | | 151 | 31.757 | 16.185 | −82.281 | 1.00 | 20.64 | C |
| ATOM | 1069 | C | PHE | | 151 | 35.202 | 21.124 | −84.923 | 1.00 | 23.15 | C |
| ATOM | 1070 | O | PHE | | 151 | 36.380 | 21.258 | −84.590 | 1.00 | 24.15 | O |
| ATOM | 1071 | N | THR | | 152 | 34.487 | 22.093 | −85.487 | 1.00 | 21.62 | N |
| ATOM | 1072 | CA | THR | | 152 | 35.015 | 23.439 | −85.729 | 1.00 | 22.22 | C |
| ATOM | 1073 | C | THR | | 152 | 35.454 | 23.660 | −87.200 | 1.00 | 19.34 | C |
| ATOM | 1074 | OG1 | THR | | 152 | 34.288 | 23.689 | −88.032 | 1.00 | 19.26 | O |
| ATOM | 1075 | CG2 | THR | | 152 | 36.414 | 22.565 | −87.671 | 1.00 | 20.08 | C |
| ATOM | 1076 | C | THR | | 152 | 33.847 | 24.394 | −85.478 | 1.00 | 21.91 | C |
| ATOM | 1077 | O | THR | | 152 | 32.705 | 23.955 | −85.303 | 1.00 | 21.69 | O |
| ATOM | 1078 | N | ASP | | 153 | 34.118 | 25.695 | −85.495 | 1.00 | 31.72 | N |
| ATOM | 1079 | CA | ASP | | 153 | 33.057 | 26.672 | −85.290 | 1.00 | 33.90 | C |
| ATOM | 1080 | CB | ASP | | 153 | 33.646 | 28.078 | −85.172 | 1.00 | 48.59 | C |
| ATOM | 1081 | CG | ASP | | 153 | 34.545 | 28.223 | −83.965 | 1.00 | 51.94 | C |
| ATOM | 1082 | OD1 | ASP | | 153 | 34.118 | 27.817 | −82.867 | 1.00 | 53.41 | O |
| ATOM | 1083 | OD2 | ASP | | 153 | 35.672 | 28.740 | −84.108 | 1.00 | 55.50 | O |
| ATOM | 1084 | C | ASP | | 153 | 32.068 | 26.627 | −86.443 | 1.00 | 33.62 | C |
| ATOM | 1085 | O | ASP | | 153 | 30.960 | 27.157 | −86.356 | 1.00 | 35.14 | O |
| ATOM | 1086 | N | SER | | 154 | 32.471 | 25.983 | −87.530 | 1.00 | 26.57 | N |
| ATOM | 1087 | CA | SER | | 154 | 31.617 | 25.892 | −88.697 | 1.00 | 25.03 | C |
| ATOM | 1088 | CB | SER | | 154 | 32.493 | 25.627 | −89.921 | 1.00 | 39.88 | C |
| ATOM | 1089 | OG | SER | | 154 | 31.766 | 25.822 | −91.106 | 1.00 | 42.24 | O |
| ATOM | 1090 | C | SER | | 154 | 30.552 | 24.794 | −88.523 | 1.00 | 24.18 | C |
| ATOM | 1091 | O | SER | | 154 | 29.434 | 24.898 | −89.043 | 1.00 | 21.98 | O |
| ATOM | 1092 | N | GLY | | 155 | 30.901 | 23.753 | −87.774 | 1.00 | 26.13 | N |
| ATOM | 1093 | CA | GLY | | 155 | 29.973 | 22.655 | −87.541 | 1.00 | 24.99 | C |
| ATOM | 1094 | C | GLY | | 155 | 30.729 | 21.359 | −87.314 | 1.00 | 23.98 | C |
| ATOM | 1095 | O | GLY | | 155 | 31.809 | 21.362 | −86.711 | 1.00 | 24.78 | O |
| ATOM | 1096 | N | ILE | | 156 | 30.171 | 20.251 | −87.794 | 1.00 | 21.74 | N |
| ATOM | 1097 | CA | ILE | | 156 | 30.808 | 18.939 | −87.658 | 1.00 | 20.36 | C |
| ATOM | 1098 | CB | ILE | | 156 | 29.858 | 17.933 | −86.977 | 1.00 | 24.45 | C |
| ATOM | 1099 | CG2 | ILE | | 156 | 30.611 | 16.652 | −86.620 | 1.00 | 26.10 | C |
| ATOM | 1100 | CG1 | ILE | | 156 | 29.286 | 18.538 | −85.694 | 1.00 | 24.15 | C |
| ATOM | 1101 | CD1 | ILE | | 156 | 28.263 | 17.651 | −84.996 | 1.00 | 23.18 | C |
| ATOM | 1102 | C | ILE | | 156 | 31.166 | 18.422 | −89.061 | 1.00 | 20.95 | C |
| ATOM | 1103 | O | ILE | | 156 | 30.324 | 18.417 | −89.965 | 1.00 | 19.91 | O |
| ATOM | 1104 | N | HIS | | 157 | 32.408 | 17.985 | −89.246 | 1.00 | 19.04 | N |
| ATOM | 1105 | CA | HIS | | 157 | 32.831 | 17.495 | −90.559 | 1.00 | 20.27 | C |
| ATOM | 1106 | CB | HIS | | 157 | 33.990 | 18.324 | −91.147 | 1.00 | 35.74 | C |
| ATOM | 1107 | CG | HIS | | 157 | 34.007 | 19.771 | −90.759 | 1.00 | 37.67 | C |
| ATOM | 1108 | CD2 | HIS | | 157 | 33.712 | 20.396 | −89.594 | 1.00 | 37.63 | C |
| ATOM | 1109 | ND1 | HIS | | 157 | 34.491 | 20.747 | −91.604 | 1.00 | 36.18 | N |
| ATOM | 1110 | CE1 | HIS | | 157 | 34.496 | 21.909 | −90.976 | 1.00 | 39.43 | C |
| ATOM | 1111 | NE2 | HIS | | 157 | 34.029 | 21.723 | −89.754 | 1.00 | 37.74 | N |
| ATOM | 1112 | C | HIS | | 157 | 33.331 | 16.043 | −90.582 | 1.00 | 20.24 | C |
| ATOM | 1113 | O | HIS | | 157 | 33.745 | 15.491 | −89.561 | 1.00 | 20.37 | O |
| ATOM | 1114 | N | ILE | | 158 | 33.289 | 15.457 | −91.777 | 1.00 | 20.22 | N |
| ATOM | 1115 | CA | ILE | | 158 | 33.829 | 14.135 | −92.035 | 1.00 | 19.41 | C |
| ATOM | 1116 | CB | ILE | | 158 | 32.831 | 13.181 | −92.710 | 1.00 | 19.81 | C |
| ATOM | 1117 | CG2 | ILE | | 158 | 33.540 | 11.878 | −93.086 | 1.00 | 20.72 | C |
| ATOM | 1118 | CG1 | ILE | | 158 | 31.670 | 12.867 | −91.772 | 1.00 | 19.97 | C |
| ATOM | 1119 | CD1 | ILE | | 158 | 30.542 | 12.114 | −92.449 | 1.00 | 20.76 | C |
| ATOM | 1120 | C | ILE | | 158 | 34.881 | 14.542 | −93.066 | 1.00 | 19.80 | C |
| ATOM | 1121 | O | ILE | | 158 | 34.553 | 15.207 | −94.051 | 1.00 | 20.12 | O |
| ATOM | 1122 | N | ILE | | 159 | 36.135 | 14.173 | −92.840 | 1.00 | 17.51 | N |
| ATOM | 1123 | CA | ILE | | 159 | 37.196 | 14.565 | −93.765 | 1.00 | 19.25 | C |
| ATOM | 1124 | CB | ILE | | 159 | 38.303 | 15.373 | −93.027 | 1.00 | 19.91 | C |
| ATOM | 1125 | CG2 | ILE | | 159 | 39.316 | 15.911 | −94.021 | 1.00 | 21.46 | C |
| ATOM | 1126 | CG1 | ILE | | 159 | 37.679 | 16.545 | −92.267 | 1.00 | 22.20 | C |
| ATOM | 1127 | CD1 | ILE | | 159 | 38.679 | 17.343 | −91.410 | 1.00 | 18.54 | C |
| ATOM | 1128 | C | ILE | | 159 | 37.855 | 13.386 | −94.479 | 1.00 | 18.91 | C |
| ATOM | 1129 | O | ILE | | 159 | 38.172 | 12.367 | −93.862 | 1.00 | 20.65 | O |
| ATOM | 1130 | N | LEU | | 160 | 38.043 | 13.536 | −95.787 | 1.00 | 19.50 | N |
| ATOM | 1131 | CA | LEU | | 160 | 38.689 | 12.506 | −96.600 | 1.00 | 20.02 | C |
| ATOM | 1132 | CB | LEU | | 160 | 37.749 | 12.011 | −97.699 | 1.00 | 19.07 | C |
| ATOM | 1133 | CG | LEU | | 160 | 38.337 | 10.959 | −98.648 | 1.00 | 20.92 | C |
| ATOM | 1134 | CD1 | LEU | | 160 | 38.496 | 9.619 | −97.905 | 1.00 | 21.52 | C |
| ATOM | 1135 | CD2 | LEU | | 160 | 37.412 | 10.783 | −99.851 | 1.00 | 19.86 | C |
| ATOM | 1136 | C | LEU | | 160 | 39.926 | 13.110 | −97.249 | 1.00 | 20.20 | C |
| ATOM | 1137 | O | LEU | | 160 | 39.834 | 14.070 | −98.013 | 1.00 | 21.45 | O |
| ATOM | 1138 | N | ARG | | 161 | 41.092 | 12.571 | −96.934 | 1.00 | 22.38 | N |
| ATOM | 1139 | CA | ARG | | 161 | 42.296 | 13.100 | −97.535 | 1.00 | 23.38 | C |
| ATOM | 1140 | CB | ARG | | 161 | 43.510 | 12.872 | −96.639 | 1.00 | 28.22 | C |
| ATOM | 1141 | CG | ARG | | 161 | 44.757 | 13.479 | −97.249 | 1.00 | 31.35 | C |
| ATOM | 1142 | CD | ARG | | 161 | 46.024 | 13.171 | −96.481 | 1.00 | 33.89 | C |
| ATOM | 1143 | NE | ARG | | 161 | 47.180 | 13.566 | −97.279 | 1.00 | 33.83 | N |
| ATOM | 1144 | CZ | ARG | | 161 | 48.446 | 13.361 | −96.933 | 1.00 | 36.18 | C |
| ATOM | 1145 | NH1 | ARG | | 161 | 48.742 | 12.762 | −95.783 | 1.00 | 35.10 | N |
| ATOM | 1146 | NH2 | ARG | | 161 | 49.419 | 13.744 | −97.752 | 1.00 | 34.40 | N |
| ATOM | 1147 | C | ARG | | 161 | 42.485 | 12.406 | −98.877 | 1.00 | 22.99 | C |
| ATOM | 1148 | O | ARG | | 161 | 42.590 | 11.181 | −98.948 | 1.00 | 23.07 | O |
| ATOM | 1149 | N | THR | | 162 | 42.511 | 13.197 | −99.942 | 1.00 | 21.36 | N |
| ATOM | 1150 | CA | THR | | 162 | 42.659 | 12.657 | −101.279 | 1.00 | 23.18 | C |
| ATOM | 1151 | CB | THR | | 162 | 41.703 | 13.371 | −102.253 | 1.00 | 23.83 | C |
| ATOM | 1152 | OG1 | THR | | 162 | 41.915 | 14.788 | −102.184 | 1.00 | 24.52 | O |
| ATOM | 1153 | CG2 | THR | | 162 | 40.262 | 13.068 | −101.892 | 1.00 | 24.17 | C |
| ATOM | 1154 | C | THR | | 162 | 44.085 | 12.733 | −101.825 | 1.00 | 23.58 | C |
| ATOM | 1155 | O | THR | | 162 | 44.439 | 11.975 | −102.723 | 1.00 | 23.23 | O |
| ATOM | 1156 | N | GLU | | 163 | 44.893 | 13.649 | −101.296 | 1.00 | 23.52 | N |
| ATOM | 1157 | CA | GLU | | 163 | 46.283 | 13.803 | −101.741 | 1.00 | 25.66 | C |
| ATOM | 1158 | CB | GLU | | 163 | 46.378 | 14.795 | −102.908 | 1.00 | 30.58 | C |
| ATOM | 1159 | CG | GLU | | 163 | 45.654 | 14.387 | −104.176 | 1.00 | 32.82 | C |
| ATOM | 1160 | CD | GLU | | 163 | 45.797 | 15.425 | −105.276 | 1.00 | 34.85 | C |
| ATOM | 1161 | OE1 | GLU | | 163 | 45.184 | 15.248 | −106.352 | 1.00 | 35.37 | O |
| ATOM | 1162 | OE2 | GLU | | 163 | 46.525 | 16.420 | −105.064 | 1.00 | 35.76 | O |
| ATOM | 1163 | C | GLU | | 163 | 47.174 | 14.319 | −100.615 | 1.00 | 26.53 | C |
| ATOM | 1164 | O | GLU | | 163 | 46.634 | 14.906 | −99.654 | 1.00 | 27.33 | O |
| ATOM | 1165 | OXT | GLU | | 163 | 48.407 | 14.164 | −100.722 | 1.00 | 29.79 | O |
| TER | 1166 | | GLU | | 163 | | | | | | |
| ATOM | 1167 | OH2 | TIP | B | 1 | 37.065 | 26.124 | −85.500 | 1.00 | 38.20 | O |
| ATOM | 1168 | OH2 | TIP | B | 2 | 37.869 | 28.739 | −86.179 | 1.00 | 56.84 | O |
| ATOM | 1169 | OH2 | TIP | B | 3 | 39.402 | 13.911 | −80.303 | 1.00 | 27.85 | O |
| ATOM | 1170 | OH2 | TIP | B | 4 | 25.496 | 19.618 | −104.419 | 1.00 | 29.09 | O |
| ATOM | 1171 | OH2 | TIP | B | 5 | 39.472 | 16.042 | −102.531 | 1.00 | 22.42 | O |
| ATOM | 1172 | OH2 | TIP | B | 6 | 34.887 | 0.262 | −93.842 | 1.00 | 26.47 | O |
| ATOM | 1173 | OH2 | TIP | B | 7 | 31.168 | 19.693 | −98.752 | 1.00 | 21.68 | O |
| ATOM | 1174 | OH2 | TIP | B | 8 | 45.776 | 8.755 | −89.647 | 1.00 | 24.32 | O |
| ATOM | 1175 | OH2 | TIP | B | 9 | 43.289 | 8.931 | −88.556 | 1.00 | 26.13 | O |
| ATOM | 1176 | OH2 | TIP | B | 10 | 31.088 | 18.908 | −109.784 | 1.00 | 26.50 | O |
| ATOM | 1177 | OH2 | TIP | B | 11 | 35.457 | 14.852 | −103.096 | 1.00 | 31.26 | O |
| ATOM | 1178 | OH2 | TIP | B | 12 | 32.512 | 3.897 | −99.210 | 1.00 | 23.08 | O |
| ATOM | 1179 | OH2 | TIP | B | 13 | 22.393 | 16.247 | −93.167 | 1.00 | 38.13 | O |
| ATOM | 1180 | OH2 | TIP | B | 14 | 29.726 | 6.333 | −87.054 | 1.00 | 29.72 | O |
| ATOM | 1181 | OH2 | TIP | B | 15 | 44.926 | 24.966 | −92.876 | 1.00 | 32.68 | O |
| ATOM | 1182 | OH2 | TIP | B | 16 | 22.894 | 10.216 | −91.185 | 1.00 | 31.32 | O |
| ATOM | 1183 | OH2 | TIP | B | 17 | 29.107 | 17.255 | −106.663 | 1.00 | 30.24 | O |
| ATOM | 1184 | OH2 | TIP | B | 18 | 37.057 | 1.007 | −89.526 | 1.00 | 25.11 | O |
| ATOM | 1185 | OH2 | TIP | B | 19 | 20.970 | 21.419 | −93.904 | 1.00 | 32.11 | O |
| ATOM | 1186 | OH2 | TIP | B | 20 | 39.881 | 17.796 | −82.737 | 1.00 | 42.56 | O |
| ATOM | 1187 | OH2 | TIP | B | 21 | 34.645 | 2.593 | −84.241 | 1.00 | 23.58 | O |
| ATOM | 1188 | OH2 | TIP | B | 22 | 19.532 | 13.900 | −101.840 | 1.00 | 38.82 | O |
| ATOM | 1189 | OH2 | TIP | B | 23 | 41.754 | −5.394 | −83.388 | 1.00 | 24.68 | O |
| ATOM | 1190 | OH2 | TIP | B | 24 | 35.589 | 7.272 | −83.796 | 1.00 | 31.95 | O |
| ATOM | 1191 | OH2 | TIP | B | 25 | 31.364 | 8.255 | −85.956 | 1.00 | 44.18 | O |
| ATOM | 1192 | OH2 | TIP | B | 26 | 47.929 | 17.330 | −87.402 | 1.00 | 30.73 | O |
| ATOM | 1193 | OH2 | TIP | B | 28 | 23.283 | 18.659 | −93.681 | 1.00 | 34.98 | O |
| ATOM | 1194 | OH2 | TIP | B | 29 | 20.270 | 12.799 | −94.753 | 1.00 | 35.08 | O |
| ATOM | 1195 | OH2 | TIP | B | 30 | 42.049 | 4.938 | −91.169 | 1.00 | 27.29 | O |
| ATOM | 1196 | OH2 | TIP | B | 31 | 50.300 | 16.102 | −100.320 | 1.00 | 43.56 | O |
| ATOM | 1197 | OH2 | TIP | B | 32 | 18.990 | 34.588 | −95.754 | 1.00 | 42.95 | O |
| ATOM | 1198 | OH2 | TIP | B | 33 | 43.024 | 16.973 | −81.210 | 1.00 | 56.78 | O |
| ATOM | 1199 | OH2 | TIP | B | 34 | 16.466 | 35.597 | −98.387 | 1.00 | 46.94 | O |
| ATOM | 1200 | OH2 | TIP | B | 36 | 48.313 | 17.969 | −102.901 | 1.00 | 48.26 | O |
| ATOM | 1201 | OH2 | TIP | B | 37 | 19.619 | 19.190 | −99.935 | 1.00 | 45.81 | O |
| ATOM | 1202 | OH2 | TIP | B | 38 | 15.428 | 27.959 | −93.465 | 1.00 | 48.10 | O |
| ATOM | 1203 | OH2 | TIP | B | 39 | 18.933 | 17.576 | −79.666 | 1.00 | 48.75 | O |
| ATOM | 1204 | OH2 | TIP | B | 40 | 23.029 | 8.242 | −86.377 | 1.00 | 49.04 | O |
| ATOM | 1205 | OH2 | TIP | B | 42 | 46.906 | 4.177 | −94.935 | 1.00 | 45.70 | O |
| ATOM | 1206 | OH2 | TIP | B | 43 | 19.911 | 13.874 | −86.366 | 1.00 | 40.92 | O |
| ATOM | 1207 | OH2 | TIP | B | 44 | 37.973 | −1.485 | −88.793 | 1.00 | 23.99 | O |
| ATOM | 1208 | OH2 | TIP | B | 45 | 39.343 | 15.086 | −104.928 | 1.00 | 27.19 | O |
| ATOM | 1209 | OH2 | TIP | B | 46 | 20.852 | 9.599 | −95.956 | 1.00 | 62.80 | O |
| ATOM | 1210 | OH2 | TIP | B | 47 | 40.525 | 5.715 | −93.227 | 1.00 | 24.44 | O |
| ATOM | 1211 | OH2 | TIP | B | 48 | 17.749 | 17.649 | −90.193 | 1.00 | 40.14 | O |
| ATOM | 1212 | OH2 | TIP | B | 50 | 41.006 | −9.309 | −84.018 | 1.00 | 33.61 | O |
| ATOM | 1213 | OH2 | TIP | B | 51 | 42.068 | −8.017 | −79.417 | 1.00 | 59.93 | O |
| ATOM | 1214 | OH2 | TIP | B | 53 | 37.528 | 20.043 | −107.955 | 1.00 | 44.18 | O |
| ATOM | 1215 | OH2 | TIP | B | 55 | 39.439 | 12.383 | −105.120 | 1.00 | 44.54 | O |
| ATOM | 1216 | OH2 | TIP | B | 56 | 50.462 | 9.178 | −96.912 | 1.00 | 42.35 | O |
| ATOM | 1217 | OH2 | TIP | B | 58 | 19.361 | 16.611 | −93.332 | 1.00 | 50.02 | O |
| ATOM | 1218 | OH2 | TIP | B | 59 | 49.469 | 11.983 | −92.574 | 1.00 | 44.09 | O |
| ATOM | 1219 | OH2 | TIP | B | 60 | 38.286 | 30.094 | −82.714 | 1.00 | 48.37 | O |
| ATOM | 1220 | OH2 | TIP | B | 61 | 43.411 | 19.888 | −82.885 | 1.00 | 55.87 | O |
| ATOM | 1221 | OH2 | TIP | B | 62 | 30.427 | 10.544 | −82.269 | 1.00 | 38.77 | O |
| ATOM | 1222 | OH2 | TIP | B | 63 | 36.120 | −2.889 | −90.071 | 1.00 | 44.94 | O |
| ATOM | 1223 | OH2 | TIP | B | 64 | 19.472 | 11.287 | −87.779 | 1.00 | 49.41 | O |
| ATOM | 1224 | OH2 | TIP | B | 66 | 13.631 | 33.639 | −97.998 | 1.00 | 38.53 | O |
| ATOM | 1225 | OH2 | TIP | B | 67 | 32.535 | −0.911 | −83.821 | 1.00 | 30.38 | O |
| ATOM | 1226 | OH2 | TIP | B | 68 | 33.471 | 6.370 | −99.916 | 1.00 | 35.36 | O |
| ATOM | 1227 | OH2 | TIP | B | 69 | 38.816 | 6.344 | −96.551 | 1.00 | 25.15 | O |
| ATOM | 1228 | OH2 | TIP | B | 71 | 45.004 | 27.148 | −88.921 | 1.00 | 37.88 | O |
| ATOM | 1229 | OH2 | TIP | B | 72 | 49.231 | 21.517 | −84.437 | 1.00 | 42.41 | O |
| ATOM | 1230 | OH2 | TIP | B | 73 | 46.459 | −5.431 | −83.135 | 1.00 | 43.09 | O |
| ATOM | 1231 | OH2 | TIP | B | 74 | 36.089 | 6.660 | −96.786 | 1.00 | 27.41 | O |
| ATOM | 1232 | OH2 | TIP | B | 77 | 49.839 | 15.344 | −87.629 | 1.00 | 43.91 | O |
| ATOM | 1233 | OH2 | TIP | B | 79 | 37.577 | 20.201 | −82.541 | 1.00 | 30.78 | O |
| ATOM | 1234 | OH2 | TIP | B | 82 | 28.801 | 2.482 | −85.775 | 1.00 | 44.90 | O |
| ATOM | 1235 | OH2 | TIP | B | 84 | 30.581 | 21.992 | −84.159 | 1.00 | 34.07 | O |
| ATOM | 1236 | OH2 | TIP | B | 86 | 45.141 | 28.886 | −86.683 | 1.00 | 37.05 | O |
| ATOM | 1237 | OH2 | TIP | B | 87 | 42.296 | 24.701 | −101.906 | 1.00 | 42.10 | O |
| ATOM | 1238 | OH2 | TIP | B | 89 | 31.576 | 5.059 | −82.942 | 1.00 | 62.83 | O |
| TER | 1239 | | TIP | B | 89 | | | | | | |
| ATOM | 1240 | O1 | PGB | C | 180 | 33.597 | 3.812 | −81.362 | 1.00 | 41.90 | O |
| ATOM | 1241 | C2 | PGB | C | 180 | 34.348 | 2.942 | −80.636 | 1.00 | 42.07 | C |
| ATOM | 1242 | C3 | PGB | C | 180 | 34.065 | 1.486 | −81.048 | 1.00 | 38.61 | C |
| ATOM | 1243 | O4 | PGB | C | 180 | 34.810 | 1.144 | −82.101 | 1.00 | 37.47 | O |
| ATOM | 1244 | C5 | PGB | C | 180 | 35.357 | −0.070 | −81.989 | 1.00 | 31.75 | C |
| ATOM | 1245 | C6 | PGB | C | 180 | 36.199 | −0.358 | −83.248 | 1.00 | 29.30 | C |
| ATOM | 1246 | O7 | PGB | C | 180 | 35.413 | −0.258 | −84.351 | 1.00 | 29.20 | O |
| ATOM | 1247 | C8 | PGB | C | 180 | 36.097 | −0.485 | −85.493 | 1.00 | 24.87 | C |
| ATOM | 1248 | C9 | PGB | C | 180 | 35.144 | −0.360 | −86.704 | 1.00 | 25.69 | C |
| ATOM | 1249 | O10 | PGB | C | 180 | 34.261 | 0.652 | −86.495 | 1.00 | 24.77 | O |
| ATOM | 1250 | C11 | PGB | C | 180 | 33.399 | 0.809 | −87.522 | 1.00 | 24.67 | C |
| ATOM | 1251 | C12 | PGB | C | 180 | 32.469 | 2.010 | −87.230 | 1.00 | 26.91 | C |
| ATOM | 1252 | O13 | PGB | C | 180 | 33.125 | 3.184 | −87.399 | 1.00 | 25.33 | O |
| ATOM | 1253 | C14 | PGB | C | 180 | 32.381 | 4.208 | −86.939 | 1.00 | 28.12 | C |
| ATOM | 1254 | C15 | PGB | C | 180 | 33.280 | 5.432 | −86.597 | 1.00 | 26.06 | C |
| ATOM | 1255 | O16 | PGB | C | 180 | 34.151 | 5.712 | −87.590 | 1.00 | 28.17 | O |
| ATOM | 1256 | C17 | PGB | C | 180 | 34.869 | 6.738 | −87.233 | 1.00 | 29.17 | C |
| ATOM | 1257 | C18 | PGB | C | 180 | 35.893 | 7.149 | −88.294 | 1.00 | 30.74 | C |
| ATOM | 1258 | O19 | PGB | C | 180 | 36.761 | 6.174 | −88.418 | 1.00 | 31.21 | O |
| ATOM | 1259 | C20 | PGB | C | 180 | 37.403 | 6.346 | −89.539 | 1.00 | 32.26 | C |
| ATOM | 1260 | C21 | PGB | C | 180 | 38.505 | 5.661 | −90.335 | 1.00 | 34.94 | C |
| ATOM | 1261 | O22 | PGB | C | 180 | 38.048 | 4.486 | −90.698 | 1.00 | 36.18 | O |
| TER | 1262 | | PGB | C | 180 | | | | | | |
| ATOM | 1263 | O1 | CRY | $ | 1101 | 37.898 | 25.387 | −91.926 | 1.00 | 20.17 | O |
| ATOM | 1264 | O2 | CRY | $ | 1101 | 34.697 | 24.657 | −93.373 | 1.00 | 20.17 | O |
| ATOM | 1265 | O3 | CRY | $ | 1101 | 35.992 | 25.052 | −95.950 | 1.00 | 20.17 | O |
| ATOM | 1266 | C1 | CRY | $ | 1101 | 36.903 | 24.469 | −92.384 | 1.00 | 20.17 | C |
| ATOM | 1267 | C2 | CRY | $ | 1101 | 36.083 | 25.052 | −93.517 | 1.00 | 20.17 | C |
| ATOM | 1268 | C3 | CRY | $ | 1101 | 36.682 | 24.557 | −94.810 | 1.00 | 20.17 | C |
| ATOM | 1269 | C1 | CIT | $ | 1003 | 30.016 | 28.736 | −90.970 | 1.00 | 20.17 | C |
| ATOM | 1270 | O1 | CIT | $ | 1003 | 29.414 | 28.081 | −90.022 | 1.00 | 20.17 | O |
| ATOM | 1271 | O2 | CIT | $ | 1003 | 30.673 | 29.785 | −90.703 | 1.00 | 20.17 | O |
| ATOM | 1272 | C2 | CIT | $ | 1003 | 29.868 | 28.186 | −92.378 | 1.00 | 20.17 | C |
| ATOM | 1273 | C3 | CIT | $ | 1003 | 30.917 | 28.433 | −93.455 | 1.00 | 20.17 | C |
| ATOM | 1274 | O7 | CIT | $ | 1003 | 30.314 | 29.340 | −94.480 | 1.00 | 20.17 | O |
| ATOM | 1275 | C4 | CIT | $ | 1003 | 31.340 | 27.135 | −94.080 | 1.00 | 20.17 | C |
| ATOM | 1276 | C5 | CIT | $ | 1003 | 32.332 | 27.138 | −95.191 | 1.00 | 20.17 | C |
| ATOM | 1277 | O3 | CIT | $ | 1003 | 33.171 | 28.011 | −95.494 | 1.00 | 20.17 | O |
| ATOM | 1278 | O4 | CIT | $ | 1003 | 32.305 | 26.147 | −95.937 | 1.00 | 20.17 | O |
| ATOM | 1279 | C6 | CIT | $ | 1003 | 32.174 | 29.273 | −93.057 | 1.00 | 20.17 | C |
| ATOM | 1280 | O5 | CIT | $ | 1003 | 32.708 | 28.874 | −91.920 | 1.00 | 20.17 | O |
| ATOM | 1281 | O6 | CIT | $ | 1003 | 32.467 | 30.177 | −93.904 | 1.00 | 20.17 | O |

### Example 5

### Sequence Alignment of Pin1 and Pin1 Like Polypeptides to Determine Conserved Residues

The following sequence alignment was done using Eukaryotic metazoa species, where presumably the Pin1 protein serves the same purpose in each organism. Residues determined not to be conserved are not absolutely critical to the unique characteristics of Pin1 in these systems. Additional alignments that build up the specificity template include: i) comparing all human proline isomerases (hPin1, hPin4, all cylcophilins, all FKBP's, and any other misc. theoretical proteins); ii) comparing Pin1 with distantly related phosphoserine-proline specific proline isomerases; iii) others—depending on the question to be addressed.

Sequence alignment of Eukaryotic Metazoa: Cross-species alignment indicates residues essential for general Pin1 function.

[image]

[image]

### Equivalents

Those skilled in the art will recognize, or be able to ascertain using no more than routine experimentation, many equivalents to the specific embodiments and methods described herein. Such equivalents are intended to be encompassed by the scope of the following claims.

We claim:

1. A method of designing specific inhibitors of a Pin1 polypeptide, comprising the steps of:
(a) determining the three-dimensional structures of crystallized Pin1 polypeptides in complex with small molecules;
(b) employing the three-dimensional structures from step (a) to form an interaction template;
(c) employing sequence and structural alignments to identify conserved amino acids among proline isomerases and distinguishing those amino acids that are specific to Pin1 to define a specificity template;
(d) using the interaction template and the specificity template in conjunction with molecular modeling to design inhibitors specific to Pin1 or Pin1-like proline isomerases; and
(e) determining the affinity and specificity of the inhibitors to Pin1.

2. A method for designing or selecting an inhibitor of a Pin1 polypeptide, comprising the steps of:
a) using a three dimensional structure of a crystallized Pin1 polypeptide having a substitution, deletion or insertion of one or more amino acids of the sequence set forth in SEQ ID NO:1 to form a Pin1 interaction template; and
b) employing the Pin1 interaction template to design or select the Pin1 inhibitor.

3. The method of claim 2, wherein the crystallized Pin1 polypeptide has a substitution, insertion or deletion of one or more amino acids within the WW-domain of the Pin-1 polypeptide.

4. The method of claim 3, wherein the crystallized Pin1 polypeptide has one amino acid substitution.

5. The method of claim 4, wherein the amino acid substitution of the crystallized Pin1 polypeptide is at position 14 of the amino acid sequence set forth in SEQ ID NO:1.

6. The method of claim 5, wherein the amino acid substitution at position 14 of the crystallized Pin1 polypeptide is a nonpolar amino acid.

7. The method of claim 6, wherein the nonpolar amino acid at position 14 is alanine.

8. The method of claim 2, wherein the crystallized Pin1 polypeptide further comprises a small molecule within the active site of the Pin1 polypeptide.

9. The method of claim 2, wherein the three dimensional structure of more than one crystallized Pin1 polypeptide is used to form the Pin1 interaction template.

10. The method of claim 2, wherein the three dimensional structure of more than one crystallized Pin1 polypeptide comprising a small molecule within the active site of the Pin1 polypeptide is used to form the Pin1 interaction template.

11. The method of claim 2, further comprising the step of using a Pin1 specificity template.

12. The method of claim 2, further comprising the step of contacting the inhibitor with a Pin1 polypeptide in the presence of a substrate to determine the ability of the inhibitor to inhibit the Pin1 polypeptide.

13. The method of claim 2, 7 or 8, further comprising the step of determining the affinity and specificity of the inhibitor.

14. The method according to claims 1 or 2, wherein said inhibitor is designed or selected de novo.

15. The method according to claims 1 or 2, wherein said inhibitor is designed or selected from a known inhibitor.

16. The method according to claims 1 or 2, wherein said three-dimensional structure is used to design or select chemical entities or fragments that bind to at least one of the following portions of the Pin1 active site: the hydrophobic pocket, the cysteine/scrine valley, the phosphate binding pocket, the substrate grove and the lip regions.

17. The method of claim 16, wherein the identified chemical entities or fragments are covalently linked.

18. The method according to anyone of claims 1 or 2 wherein said inhibitor is a competitive inhibitor of Pin1.

19. The method according to anyone of claims 1 or 2 wherein said inhibitor is a non-competitive or uncompetitive inhibitor of Pin1.

20. A Pin1 polypeptide in the crystallized form having a substitution, insertion or deletion of one or more amino acids of the amino acid sequence set forth in SEQ ID NO:1, wherein the active site of the Pin1 polypeptide is accessible to solvent and available for interaction with an inhibitor in a crystallized form.

21. The crystallized Pin1 polypeptide of claim 20 wherein the substitution, insertion or deletion of one or more amino acids is within the WW-domain of the Pin-i polypeptide.

22. The crystallized Pin1 polypeptide of claim 21 having one amino acid substitution.

23. The crystallized Pin1 polypeptide of claim 21, having an amino acid substitution at position 14 of the amino acid sequence set forth in SEQ ID NO:1.

24. The crystallized Pin1 polypeptide of claim 23, wherein the amino acid substitution at position 14 is a nonpolar amino acid.

25. The crystallized Pin1 polypeptide of claim 24, wherein the nonpolar amino acid at position 14 is alanine (SEQ ID NO:2).

26. The crystallized Pin1 polypeptide of any one of claims 20-25, wherein the polypeptide crystallizes within 7 days.

27. The crystallized Pin1 polypeptide of claim 26, wherein the polypeptide crystallizes within 3 days.

28. The crystallized Pin1 polypeptide of claim 26, wherein the polypeptide crystallizes within 2 days.

29. The crystallized Pin1 polypeptide of claim 26, wherein the polypeptide crystallizes within 24 hours.

30. The crystallized Pin 1 polypeptide of any of claims 20-25, having a resolution of about 3 Å or less.

31. The crystallized Pin1 polypeptide of claim 30, having a resolution of 2 Å or less.

32. The crystallized Pin1 polypeptide of claim 25, wherein the three dimensional structure of the polypeptide has the space group P3₁21.

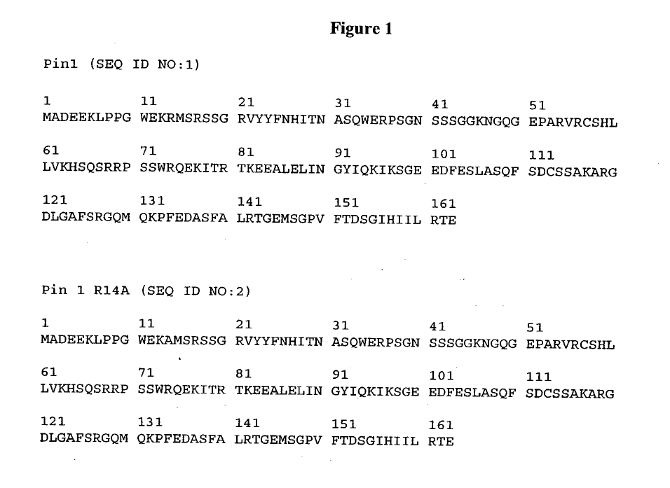

33. The crystallized Pin1 polypeptide of claim 32 defined by the atomic coordinates set forth in FIG. 1.

34. A crystallized fragment of the Pin1 polypeptide of any of claims 20-25.

35. The crystallized fragment of claim 34, wherein the fragment comprises the active site of the Pin1 polypeptide.

36. The crystallized Pin1 polypeptide of claim 20, further comprising a Pin1 polypeptide inhibitor in the active site.

37. The Pin1 polypeptide inhibitor of claim 36, wherein the inhibitor is a small molecule.

38. A method for designing an inhibitor of a second peptidyl/prolyl isomerase comprising the steps of:
(a) providing a first peptidyl/prolyl isomerase having a known three-dimensional structure;
(b) identifying amino acids in the active site of said first peptidyl/prolyl isomerase that form close contacts with a compound know to bind to said active site;
(c) employing protein alignment means to identify in a second peptidyl/prolyl isomerase one or more amino acids that align with, but differ in identity from, said close contact amino acids in said first peptidyl/prolyl isomerase;
(d) altering an amino acid in the active site of said second peptidyl/prolyl isomerase identified in step (c) to produce a mutant second peptidyl/prolyl isomerase;
(e) determining that said compound binds with at least 10-fold greater affinity to said mutant second peptidyl/prolyl isomerase than to said second peptidyl/prolyl isomerase, and
(f) using molecular modeling means to determine how to modify said compound to design an inhibitor of said second peptidyl/prolyl isomerase, wherein said inhibitor binds to said second peptidyl/prolyl isomerase with at least 10-fold greater affinity than said compound binds to said peptidyl/prolyl isomerase, respectively.

39. The method according to claim 38, wherein said first peptidyl/prolyl isomerase is Pin1 having the amino acid sequence set forth in SEQ ID NO:1.

40. The method according to claim 38, wherein said second peptidyl/prolyl isomerase is selected from the group consisting of hCyP-A, hCyP-B, hCyP-C, NKCA, hFKBP-12, hFKBP-13, Par14 and hFKBP-25.
